(19) United States
(12) Patent Application Publication
(10) Pub. No.: US 2026/0078120 A1
(43) Pub. Date: Mar. 19, 2026

(54) SUBSTITUTED PYRIDOPYRIMIDINONES

(71) Applicant: MIRATI THERAPEUTICS, INC., PRINCETON, NJ (US)

(72) Inventors: JOHN MICHAEL KETCHAM, CARY, NC (US); CHRISTOPHER RONALD SMITH, SAN DIEGO, CA (US); MATTHEW ARNOLD MARX, CHULA VISTA, CA (US); JOHN DAVID LAWSON, CARLSBAD, CA (US); STEPHEN HARWOOD, SAN DIEGO, CA (US); AARON CRAIG BURNS, SAN DIEGO, CA (US); KERSTI CADDELL-HAATVEIT, BENICIA, CA (US); KELLY ELAINE PEARSON, SAN DIEGO, CA (US); ATHENEA NOELANI GARDEN ALOIAU, SAN DIEGO, CA (US); JON KUEHLER, SAN DIEGO, CA (US); BRIANA BOBEK, SAN DIEGO, CA (US); ASHLEE HOPE WATKINS, SANTEE, CA (US); BENJAMIN DONALD JONES, CARDIFF, CA (US); ANTHONY DAVID IVETAC, SAN DIEGO, CA (US); XIAOLUN WANG, SAN DIEGO, CA (US)

(73) Assignee: MIRATI THERAPEUTICS, INC., PRINCETON, NJ (US)

(21) Appl. No.: 19/106,317

(22) PCT Filed: Aug. 25, 2023
(86) PCT No.: PCT/US2023/072945

§ 371 Date: Feb. 25, 2025

Related U.S. Application Data
(60) Provisional application No. 63/401,507, filed on Aug. 26, 2022.

(51) Int. Cl. C07D 471/04; A61K 31/519; A61K 31/5377; A61K 31/5386; A61K 31/541; A61K 31/55; A61K 31/551; A61K 31/675; A61P 35/00; C07D 519/00; C07F 9/6561

(52) U.S. Cl. CPC C07D 471/04; A61K 31/519; A61K 31/5377; A61K 31/5386; A61K 31/541; A61K 31/55; A61K 31/551; A61K 31/675; A61P 35/00; C07D 519/00; C07F 9/6561

(57) ABSTRACT

Disclosed herein are substituted pyridopyrimidinones, methods for their preparation, and use thereof.

### CROSS-REFERENCE TO RELATED APPLICATIONS

This application claims the benefit of priority of U.S. Provisional Patent Application No. 63/401,507, filed Aug. 26, 2022, the disclosure of which is incorporated herein by reference in its entirety.

### FIELD

This disclosure is directed to substituted pyridopyrimidinones, and more particularly to such compounds that are inhibitors of phosphoinositide 3-kinase (PI3K) and therefore useful in the treatment of diseases or disorders associated with PI3K modulation. The disclosure is directed toward compounds and compositions which inhibit PI3K, methods of treating a disease or disorder associated with PI3K (e.g., CLOVES syndrome (congenital lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer), and methods of using PI3K inhibitors in combination with one or more additional disorder or cancer therapy.

### BACKGROUND

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer, inflammatory disorders, metabolic disorders, vascular and neuronal diseases.

Kinases represent a class of important signaling molecules. Kinases can generally be classified into protein kinases and lipid kinases, and certain kinases exhibit dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates on tyrosine residues (e.g., erb2, PDGF receptor, EGF receptor, VEGF receptor, src, abl), serine/threonine kinases which predominantly phosphorylate substrates on serine and/or threonine residues (e.g., mTORC1, mTORC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids and lipid-derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in or associated with cell membranes. Examples of such enzymes include phosphoinositide(s) kinases (such as PI3-kinases, PI4-Kinases), diacylglycerol kinases, and sphingosine kinases.

The phosphoinositide 3-kinases (PI3Ks) signaling pathway is one of the most highly mutated systems in human cancers. PI3K signaling is involved in many other disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

PI3Ks are members of a unique and conserved family of intracellular lipid kinases that phosphorylate the 3′—OH group on phosphatidylinositols or phosphoinositides. The PI3K family comprises 15 kinases with distinct substrate specificities, expression patterns, and modes of regulation. The class I PI3Ks (pi 10a, pi 10b, pi 106, and pi 10 g) are typically activated by tyrosine kinases or G-protein coupled receptors to generate PIP3, which engages downstream effectors such as those in the pathways of Akt/PDKI, mTOR, the Tec family kinases, and the Rho family GTPases. The class II and III PI3-Ks play a key role in intracellular trafficking through the synthesis of PI(3)P and PI(3,4) P2.

The PI3K isoforms have been implicated, for example, in a variety of human cancers and disorders. Mutations in the gene coding for PI3K isoforms or mutations which lead to upregulation of a PI3K isoform are believed to occur in many human cancers. Mutations in the gene coding for a PI3K isoform are point mutations clustered within several hotspots in helical and kinase domains. Because of the high rate of PI3K mutations, targeting of this pathway may provide valuable therapeutic opportunities.

Genetic alterations in genes in PI3K signaling are believed to be involved in a range of cancers such as endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The alpha (a) isoform of PI3K has been implicated, for example, in a variety of human cancers. Angiogenesis has been shown to selectively require the alpha (α) isoform of PI3K in the control of endothelial cell migration. Mutations in the gene coding for PI3Kα or mutations which lead to upregulation of PI3Kα are believed to occur in many human cancers such as lung, stomach, endometrial, ovarian, bladder, breast, colon, brain, prostate, and skin cancers. Mutations in the gene coding for PI3Kα are point mutations clustered within several hotspots in helical and kinase domains, such as E542K, E545K, and H1047R. Many of these mutations have been shown to be oncogenic gain-of-function mutations. Because of the high rate of PI3Kα mutations, targeting of this pathway may provide valuable therapeutic opportunities. While other PI3K isoforms such as PI3Kδ or PI3Kγ are expressed primarily in hematopoietic cells, PI3Kα, along with PI3 Kβ, is expressed constitutively.

Due to the central role of PI3Kα in regulating organismal glucose homeostasis, PI3K inhibition in patients often gives rise to hyperglycemia and/or hyperinsulinemia. High levels of circulating insulin could potentially be mitogenic and/or antiapoptotic for cancer cells and thus negate the antiproliferative effects of PI3K inhibitors.

In the setting of cancer with mutated PI3Kα, one way to overcome the problem of compensatory production of insulin and/or glucose upon systemic PI3Kα inhibition would be to develop inhibitors with enhanced selectivity for mutant PI3Kα over wild-type PI3Kα. This would create an increased window for drug dosing to selectively inhibit the pathologic signaling of mutant PI3Kα in the cancer cells without affecting the wild-type PI3Kα in the host tissues that control systemic metabolism, thus limiting toxicities and permitting higher doses and more complete inhibition of the drug target.

Existing PI3Kα inhibitors are nearly equipotent to wild-type and mutant PI3Kα. Mutant selective inhibitors have been elusive due to the PI3Kα mutations location far from the active site. As such, inhibitors which target a second, peripheral binding pocket near a known mutation (e.g., H1047R) may provide a route to selective PI3Kα inhibition. Thus, targeting a mutated, peripheral binding pocket of PI3Kα, may in turn provide a valuable therapeutic target for drug development.

As such, kinases, for example lipid kinases such as PI3Ks, are prime targets for drug development.

### SUMMARY

In one aspect, the present disclosure provides compounds of Formula (I) and pharmaceutically acceptable salts thereof, and prodrugs, solvates, hydrates, isomers, deuterated forms, and tautomers thereof:

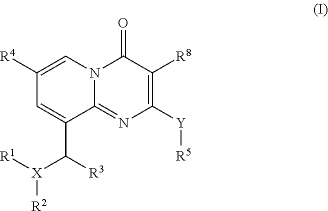

wherein:
   revised to X is N, O, or S;
   when X is N, R1 is H, C₁-C₃ alkyl, or C₃-C₆ cycloalkyl;
   when X is O or S, R1 is absent;
   R² is phenyl, 5-6 membered heteroaryl, 4-6 membered heterocyclyl, —C(O)—(C₂-C₄ alkyl), —C(O)—(C₂-C₄ alkenyl), —(C₂-C₄ alkyl)-C(O)OH or —(C₂-C₄ cycloalkyl)-C(O)OH, wherein each phenyl and heteroaryl is optionally substituted with 1-5 R⁷;
   R³ is C₁-C₃ alkyl or C₃-C₆ cycloalkyl;
   R⁴ is H, C₁-C₃ alkyl, C₃-C₈ cycloalkyl, cyano, or halo, wherein the C₁-C₃ alkyl is optionally substituted with 1-5 halo groups;
   R⁵ is C₁-C₃ alkyl, C₂-C₆ alkenyl, 6-10 membered aryl, 5-10 membered heteroaryl, C₃-C₆ cycloalkyl, or 3-9 membered heterocyclyl, wherein each alkyl, alkenyl, aryl, heteroaryl, cycloalkyl, and heterocyclyl is optionally substituted with 1-5 R⁶, and wherein heterocyclyl is optionally unsaturated;
   R⁸ is R^{A}, halogen, or cyano,
   Y is a bond, NR10, S or O;
wherein:
   each R⁶ is independently —R^{A}, —OR^{A}, —C(O)OR^{A}, oxo, cyano, halo, (C₁-C₃ alkyl)-OR^{A}, —C(O)N(R^{A})₂, —C(O)R¹¹, —N(R^{A})₂, —N(R^{A})C(O)R^{A}, —SO₂R^{A}, —R^{C}, or two geminal R⁶ together with the carbon to which they are attached form a spirocyclic C₃-C₆ cycloalkyl or 3-12 membered heterocyclyl, or two vicinal R⁶ together with the carbon to which they are attached form a fused C₁₀-C₁₄ aryl, C₈-C₁₂ cycloalkyl or C₃-C₁₂ heterocyclyl;
      wherein each spirocyclic group and fused group is optionally substituted with R^{B}, —OR^{B}, —C(O)OR^{B}, (C₁-C₃ alkyl)-OR^{B}, —C(O) N(R^{B})₂, oxo, cyano, halo,
      wherein each R^{B} is independently H, C₁-C₆ alkyl, C₃-C₆ cycloalkyl or C₃-C₆ cycloalkyl(C₁-C₆) alkyl;
   R^{C} represents C₃-C₆ cycloalkyl, C₃-C₁₂ heterocyclyl, C₃-C₁₂ heterocyclyl(C₁-C₆) alkyl, phenyl, phenyl(C₁-C₂) alkyl, 5-10 membered heteroaryl, or 5-10 membered heteroaryl(C₁-C₂) alkyl, wherein each cycloalkyl, heterocycyclyl, phenyl and heteroaryl is optionally substituted with 1-5 of R⁹,
   each R⁷ is independently cyano, halo, oxo, C₁-C₄ alkyl, —OR^{A}, —C(O)OR^{A}, (C₁-C₃ alkyl)-C(O)OR^{A}, (C₁-C₃ alkyl)-OR^{A}, —C(O)N(R^{A})₂, —P(O) (OH) R^{A} or two geminal R⁷ together with the carbon to which they are attached form a C₃-C₆ cycloalkyl or 3-9 membered heterocyclyl;
each R⁹ is independently hydroxy, halogen, cyano, C₁-C₆ alkyl, C₁-C₆ alkoxy, halo(C₁-C₆) alkyl, or—SO₂N(R¹°) 2;
each R¹⁰ is independently hydrogen or C₁-C₆ alkyl;
R¹¹ is C₁-C₆ alkyl, C₃-C₆ cycloalkyl(C₁-C₆) alkyl, C₃-C₆ cycloalkyl, or phenyl, each of which is optionally substituted with 1-5 R⁷;
each R^{A} is independently H, C₁-C₆ alkyl, C₃-C₆ cycloalkyl, C₃-C₆ cycloalkyl(C₁-C₆) alkyl or two geminal R^{A} together with the carbon to which they are attached form a C₃-C₆ cycloalkyl or 3-9 membered heterocyclyl; and
wherein, unless otherwise indicated, each alkyl and cycloalkyl group is unsubstituted, substituted with 1-5 halo groups, perfluorinated, or is substituted by 1-3 groups selected from methyl, oxo, and carboxy; and provided R⁵ is not unsubstituted morpholinyl when Y is a bond.

In another aspect, the present disclosure provides for a pharmaceutical composition comprising a compound or salt as otherwise described herein together with a pharmaceutically acceptable carrier, excipient or diluent.

In another aspect, the present disclosure provides for a method of treating a disease or disorder associated with modulation of phosphoinositide 3-kinase (PI3K), comprising administering to a patient in need thereof a therapeutically effective amount of a compound of any as otherwise described herein or a pharmaceutical composition as otherwise described herein.

In another aspect, the present disclosure provides for a method of inhibiting phosphoinositide 3-kinase (PI3K), comprising administering to a patient in need thereof a therapeutically effective amount of a compound as otherwise described herein or a pharmaceutical composition as otherwise described herein.

In another aspect, the present disclosure provides for a method of treating cancer or a disorder, the method comprising administering to a patient in need thereof a therapeutically effective amount of a compound as otherwise described herein or a pharmaceutical composition as otherwise described herein.

The compounds disclosed herein selectively bind to H1047R-mutated PI3Kα and not to wild-type PI3Kα.

Other aspects and embodiments of the disclosure are evident in view of the detailed description provided herein.

### DETAILED DESCRIPTION OF THE INVENTION

The present invention relates to inhibitors of PI3Kα. In particular, the present invention relates to compounds that inhibit PI3Kα activity, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

The term “amino” refers to —NH₂.

The term “acetyl” refers to —C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Alkyl groups may be branched or unbranched. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Exemplary alkenylene groups include, without limitation, ethenylene, propenylene, and butenylene. Exemplary alkynylene groups include, without limitation, ethynylene, propynylene, and butynylene.

The term “alkoxy” refers to —O(C₁-C₆ alkyl).

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NRx, wherein Rx is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. An exemplary aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. An exemplary aralkyl group is —(C₁-C₆) alkyl(C₆-C₁₀) aryl, including, without limitation, benzyl, phenethyl, and naphthylmethyl. For example, an arC1-Csalkyl is an aryl group covalently linked to a C₁-C₃ alkyl.

A “heterocyclyl” or “heterocyclic” or “heterocycloalkyl” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, tetrahydrofuranyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Examples of heterocyclic groups that are spiro ring systems are azaspiro[2.5]octan-6-yl, 5-azaspiro[2.4]heptan-5-yl, 6-azaspiro[3.4]octan-6-yl, 5-oxa-7-azaspiro[3.4]octan-7-yl. 5,5-dimethyl-4-oxa-7-azaspiro[2.5]octan-7-yl, and 7,7-dimethyl-5-azaspiro[2.5]octan-5-yl. Specifically excluded from the scope of this term are compounds having adjacent ring O and/or S atoms. The heterocyclic groups can be attached to a parent group (i.e., the point of attachment) via any ring atom, including one of the heteroatoms or one of the carbon atoms, in the heterocyclic ring group. As chemically required, the heterocyclic ring may be attached to one or more other groups, for instance if operating as a bridging group. The term “heterocyclyl” also includes fused multicyclic (e.g., bicyclic) ring systems in which one or more of the fused rings is aromatic or non-aromatic, provided that at least one ring is non-aromatic contains an N, O, or S ring atom. Examples of such fused multicyclic ring systems are 3,4-dihydroisoquinolin-2(1H)-yl, isoindolinyl, indolinyl, indolin-2-yl, 2,3-dihydrobenzofuran-2-yl and 2,3,4,5-tetrahydrobenzo[d]oxazol-2-yl. Each of these examples is a 9- or 10 membered heterocyclyl.

As used herein, the term “heteroaryl” refers to a group having 5 to 14 ring atoms, preferably 5, 6, 10, 13 or 14 ring atoms; having 6, 10, or 14 IT electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom. The heteroaryl groups can be attached to a parent group (i.e., the point of attachment) via any ring atom, including one of the heteroatoms or one of the carbon atoms, in the heteroaryl ring group. As chemically required, the heteroaryl may be attached to one or more other groups, for instance if operating as a bridging group.

Examples of heteroaryl groups include acridinyl, azocinyl, benzimidazolyl, benzofuranyl, benzo[d]oxazol-2(3H)-one, 2H-benzo[b][1,4]oxazin-3(4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has multiple non-hydrogen substituents, for example from one to five, or from one to four, or from one to three, or one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Exemplary haloalkyls are trifluoromethyl, difluoromethyl, flurochloromethyl, chloromethyl, and fluoromethyl.

The term “hydroxyalkyl” refers to -alkylene-OH.

It is to be understood that each individual atom present in Formula (I) and the compounds within formula (I), may be present in the form of any of its naturally occurring isotopes, with the most abundant isotope(s) being preferred. Thus, by way of example, each individual hydrogen atom present in formula (I), or in the formulae depicted hereinafter, may be present as a 1H, 2H (deuterium; D) or 3H (tritium; T) atom, preferably 1H. Similarly, by way of example, each individual carbon atom present in formula (I), or in the formulae depicted hereinafter, may be present as a 12C, 13C or 14C atom, preferably 12C.

As used herein, “an effective amount” of a compound is an amount that is sufficient to negatively modulate or inhibit the activity of PI3Kα.

As used herein, a “therapeutically effective amount” of a compound is an amount that is sufficient to ameliorate or in some manner reduce a symptom or stop or reverse progression of a condition, or negatively modulate or inhibit the activity of PI3Kα. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect, the present disclosure provides compounds of Formula (I) and pharmaceutically acceptable salts thereof, and prodrugs, solvates, hydrates, isomers, deuterated forms, and tautomers thereof:

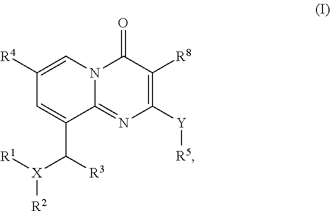

wherein:
   X is N, O, or S;
   when X is N, R¹ is H, C₁-C₃ alkyl, or C₃-C₈ cycloalkyl;
   when X is O or S, R¹ is absent;
   R² is phenyl, 5-6 membered heteroaryl, 4-6 membered heterocyclyl, —C(O)—(C₂-C₄ alkyl), —C(O)—(C₂-C₄ alkenyl), —(C₂-C₄ alkyl)-C(O)OH or —(C₂-C₄ cycloalkyl)-C(O)OH, wherein each phenyl and heteroaryl is optionally substituted with 1-5 R⁷;
   R³ is C₁-C₃ alkyl or C₃-C₈ cycloalkyl;
   R⁴ is H, C₁-C₃ alkyl, C₃-C₆ cycloalkyl, cyano, or halo, wherein the C₁-C₃ alkyl is optionally substituted with 1-5 halo groups;
   R⁵ is C₁-C₃ alkyl, C₂-C₆ alkenyl, 6-10 membered aryl, 5-10 membered heteroaryl, C₃-C₆ cycloalkyl, or 3-9 membered heterocyclyl, wherein each alkyl, alkenyl, aryl, heteroaryl, cycloalkyl, and heterocyclyl is optionally substituted with 1-5 R⁶, and wherein heterocyclyl is optionally unsaturated;
   R⁸ is R^{A}, halogen, or cyano,
   Y is a bond, NR¹⁰, S or O;
wherein:
   each R⁶ is independently —R^{A}, —OR^{A}, —C(O)OR^{A}, oxo, cyano, halo, (C₁-C₃ alkyl)-OR^{A}, —C(O)N(R^{A})₂, —C(O) R¹¹, —N(R^{A})₂, —N(R^{A}) C(O) R^{A}, —SO₂R^{A}, —R^{C}, or two geminal R⁶ together with the carbon to which they are attached form a spirocyclic C₃-C₆ cycloalkyl or 3-12 membered heterocyclyl, or two vicinal R⁶ together with the carbon to which they are attached form a fused C₁₀-C₁₄ aryl, C₈-C₁₂ cycloalkyl or C₃-C₁₂ heterocyclyl;
      wherein each spirocyclic group and fused group is optionally substituted with R^{B}, —OR^{B}, —C(O)OR^{B}, (C₁-C₃ alkyl)-OR^{B}, —C(O)N(R^{B})₂, oxo, cyano, halo,
         wherein each R^{B} is independently H, C₁-C₈alkyl, C₃-C₈ cycloalkyl or C₃-C₅ cycloalkyl(C₁-C₆) alkyl;
   R^{C} represents C₃-C₆ cycloalkyl, C₃-C₁₂ heterocyclyl, C₃-C₁₂ heterocyclyl(C₁-C₆) alkyl, phenyl, phenyl(C₁-C₂) alkyl, 5-10 membered heteroaryl, or 5-10 membered heteroaryl(C₁-C₂) alkyl, wherein each cycloalkyl, heterocycyclyl, phenyl and heteroaryl is optionally substituted with 1-5 of R⁹.
   each R⁷ is independently cyano, halo, oxo, C₁-C₄ alkyl, —OR^{A}, —C(O)OR^{A}, (C₁-C₃ alkyl)-C(O)OR^{A}, (C₁-C₃ alkyl)-OR^{A}, —C(O)N(R^{A})₂, —P(O)(OH) R^{A} or two geminal R⁷ together with the carbon to which they are attached form a C₃-C₆ cycloalkyl or 3-9 membered heterocyclyl;
each R⁹ is independently hydroxy, halogen, cyano, C₁-C₆ alkyl, C₁-C₆ alkoxy, halo(C₁-C₆) alkyl, or —SO₂N(R¹⁰)₂;
each R¹° is independently hydrogen or C₁-C₆ alkyl;
R¹¹ is C₁-C₆ alkyl, C₃-C₈ cycloalkyl(C₁-C₆) alkyl, C₃-C₆ cycloalkyl, or phenyl, each of which is optionally substituted with 1-5 R⁷;
each R^{A} is independently H, C₁-C₆ alkyl, C₃-C₆ cycloalkyl, C₃-C₆ cycloalkyl(C₁-C₆) alkyl or two geminal R^{A} together with the carbon to which they are attached form a C₃-C₆ cycloalkyl or 3-9 membered heterocyclyl; and
wherein, unless otherwise indicated, each alkyl and cycloalkyl group is unsubstituted, substituted with 1-5 halo groups, perfluorinated, or is substituted by 1-3 groups selected from methyl, oxo, and carboxy; and
   provided R⁵ is not unsubstituted morpholinyl when Y is a bond.

In certain embodiments as otherwise described herein, X is N.

In certain embodiments as otherwise described herein, Y is a bond and R⁵ is 5-7 membered heterocyclyl.

In certain embodiments as otherwise described herein, Y is a bond and R⁵ is 5-7 membered heterocyclyl selected from piperidinyl, piperazinyl, azetidinyl, and isoindolinyl, each of which is optionally substituted. Preferred R⁵ heterocyclyl groups are attached to the pyridopyrimidine ring system through a nitrogen atom in the heterocyclyl group.

In certain embodiments as otherwise described herein, Y is a bond and R⁵ is 5-6 membered heteroaryl, each of which is optionally substituted. In other embodiments, Y is a bond and R⁵ is thiophene, pyrazinyl, or pyridinyl, each of which is optionally substituted.

In certain embodiments as otherwise described herein, R¹ is H or CH₃. For example, in particular embodiments, R¹ is H. In other embodiments, R¹ is CH₃.

In certain embodiments as otherwise described herein, R² is phenyl, pyridinyl, —C(O)—CH═CH₂, or —C(O)—CH₂—Cl, wherein each phenyl or pyridinyl is optionally substituted with 1-5 R⁷.

In certain embodiments as otherwise described herein, R³ is C₁-C₃ alkyl, wherein the alkyl group is unsubstituted, substituted with 1-5 halo groups, or perfluorinated. For example, in particular embodiments, R³ is methyl, e.g., unsubstituted methyl.

In certain embodiments as otherwise described herein, R⁴ is H, methyl, ethyl, or cyclopropyl. For example, in particular embodiments, R⁴ is methyl, ethyl, or cyclopropyl, wherein each is unsubstituted.

In certain embodiments as otherwise described herein, Y is a bond.

In certain embodiments as otherwise described herein, R⁵ is 6-10 membered aryl, C₃-C₆ cycloalkyl or 3-9 membered heterocyclyl, and each is optionally substituted with 1-5 R⁶. For example, in particular embodiments, R⁵ is cyclopropyl, phenyl, piperidinyl, or isoindolinyl and each is optionally substituted with 1-5 R⁶. In various embodiments, R⁵ is not morpholino, or is not unsubstituted morpholino.

In certain embodiments as otherwise described herein, R⁸ is hydrogen, methyl, ethyl, or cyano. For example, in particular embodiments, R⁸ is methyl (e.g., unsubstituted methyl), or H.

In certain embodiments of formula (I) as otherwise described herein R¹ is H.

In certain embodiments of formula (I) as otherwise described herein R¹ is CH₃.

In certain embodiments of formula (I) as otherwise described herein R¹ is H or CH₃.

In certain embodiments of formula (I) as otherwise described herein R² is phenyl or a 5-7 membered heteroaryl, each of which is substituted with 1, 2 or 3 R⁷ groups.

In certain embodiments of formula (I) as otherwise described herein R² is phenyl or a 5-7 membered heteroaryl, each of which is substituted with 1, 2 or 3 R⁷ groups, and at least one R⁷ group is —C(O)OR^{A}.

In certain embodiments of formula (I) as otherwise described herein R² is phenyl or a 5-7 membered heteroaryl, each of which is substituted with 1, 2 or 3 R⁷ groups, and at least one R⁷ group is —C(O)OR^{A}, where R^{A} is H, methyl or ethyl.

In certain embodiments of formula (I) as otherwise described herein R² is phenyl or a 5-7 membered heteroaryl, each of which is substituted with 1, 2 or 3 R⁷ groups, and at least one R⁷ group is —C(O)OR^{A}, where R^{A} is H.

In certain embodiments of formula (I) as otherwise described herein R² is phenyl or a 5-7 membered heteroaryl, each of which is substituted with 1, 2 or 3 R⁷ groups, and at least one R⁷ group is —C(O)OR^{A}, where R^{A} is methyl or ethyl.

In certain embodiments of formula (I) as otherwise described herein R² is phenyl, pyridinyl, thienyl, thiazolyl, oxazolyl, isoxazolyl, isothiazolyl, imidazolyl, pyrazolyl, pyrazinyl, pyridazinyl or pyrimidinyl, each of which is optionally substituted with 1-5 R⁷.

In certain embodiments of formula (I) as otherwise described herein R² is phenyl or pyridinyl, each of which is optionally substituted with 1-5 R⁷.

In certain embodiments of formula (I) as otherwise described herein R² is phenyl or pyridinyl, each of which is optionally substituted with 1-5 R⁷, and at least one R⁷ group is —C(O)OR^{A}

In certain embodiments of formula (I) as otherwise described herein R² is phenyl or pyridinyl, each of which is optionally substituted with 1-5 R⁷, and at least one R⁷ group is —C(O)OR^{A}, where R^{A} is H, methyl or ethyl.

In certain embodiments of formula (I) as otherwise described herein R² is phenyl or pyridinyl, each of which is optionally substituted with 1-5 R⁷, and at least one R⁷ group is —C(O)OR^{A}, where R^{A} is H.

In certain embodiments of formula (I) as otherwise described herein R² is phenyl or pyridinyl, each of which is optionally substituted with 1-5 R⁷, and at least one R⁷ group is —C(O)OR^{A}, where R^{A} is methyl or ethyl.

In certain embodiments of formula (I) as otherwise described herein R² is phenyl which is optionally substituted with 1-5 R⁷.

In certain embodiments of formula (I) as otherwise described herein R² is phenyl which is optionally substituted with 1-5 R⁷, and at least one R⁷ group is —C(O)OR^{A}.

In certain embodiments of formula (I) as otherwise described herein R² is phenyl which is optionally substituted with 1-5 R⁷, and at least one R⁷ group is —C(O)OR^{A}, where R^{A} is H, methyl or ethyl.

In certain embodiments of formula (I) as otherwise described herein R² is phenyl which is optionally substituted with 1-5 R⁷, and at least one R⁷ group is —C(O)OR^{A}, where R^{A} is H.

In certain embodiments of formula (I) as otherwise described herein R² is phenyl which is optionally substituted with 1-5 R⁷, and at least one R⁷ group is —C(O)OR^{A}, where R^{A} is methyl or ethyl.

In certain embodiments of formula (I) as otherwise described herein R² is pyridinyl which is optionally substituted with 1-5 R⁷.

In certain embodiments of formula (I) as otherwise described herein R² is pyridinyl which is optionally substituted with 1-5 R⁷, and at least one R⁷ group is —C(O)OR^{A}.

In certain embodiments of formula (I) as otherwise described herein R² is pyridinyl which is optionally substituted with 1-5 R⁷, and at least one R⁷ group is —C(O)OR^{A}, where R^{A} is H, methyl or ethyl.

In certain embodiments of formula (I) as otherwise described herein R² is pyridinyl which is optionally substituted with 1-5 R⁷, and at least one R⁷ group is —C(O)OR^{A}, where R^{A} is H.

In certain embodiments of formula (I) as otherwise described herein R² is pyridinyl which is optionally substituted with 1-5 R⁷, and at least one R⁷ group is —C(O)OR^{A}, where R^{A} is methyl or ethyl.

In certain embodiments of formula (I) as otherwise described herein R³ is C₁-C₃ alkyl, wherein the alkyl group is unsubstituted, substituted with 1-5 halo groups, or perfluorinated.

In certain embodiments of formula (I) as otherwise described herein R³ is C₁-C₃ alkyl, wherein the alkyl group is unsubstituted, substituted with 1-5 halo groups, or perfluorinated.

In certain embodiments of formula (I) as otherwise described herein R³ is C₁-C₃ alkyl.

In certain embodiments of formula (I) as otherwise described herein R³ is ethyl or R³ is methyl.

In certain embodiments as otherwise described herein, wherein the compound is of formula (IIa), (IIb) or (IIc):

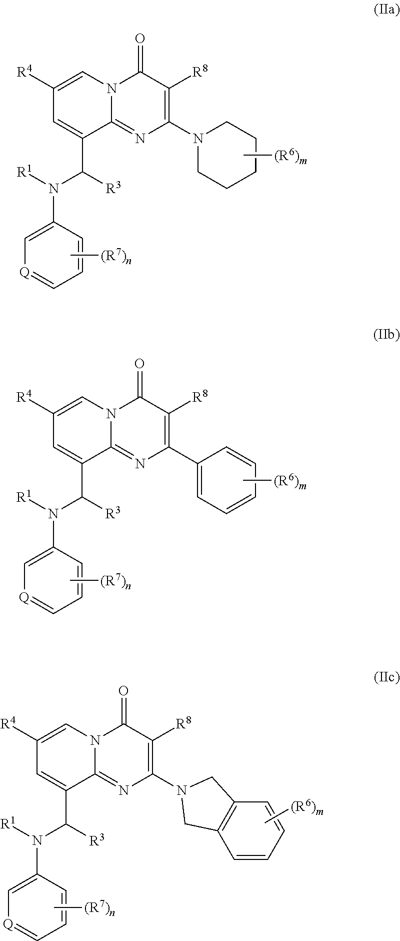

or a pharmaceutically acceptable salt thereof, wherein:
   Q is N or CH;
   R¹ is H or CH₃;
   R³ is C₁-C₃ alkyl;
   R⁴ is H, C₁-C₃ alkyl, C₃-C₆ cycloalkyl, cyano, or halo, wherein the C₁-C₃ alkyl is optionally substituted with 1-5 halo groups;
   each R⁶ is independently —R^{A}, —OR^{A}, —C(O)OR^{A}, (C₁-C₃ alkyl)-OR^{A}, —C(O)N(R^{A})₂, oxo, cyano, halo, or two geminal R⁶ together with the carbon to which they are attached form a C₃-C₆ cycloalkyl or heterocyclyl, or two vicinal R⁶ together with the carbon to which they are attached form a fused C₁₀-C₁₄ aryl or C₈-C₁₂ cycloalkyl;
   each m is 0, 1, 2, 3, or 4;
   each R⁷ is independently C₁-C₄ alkyl; —OR^{A}, —C(O)OR^{A}, (C₁-C₃ alkyl)-OR^{A}, —C(O)N(R^{A})₂, cyano, halo, or two geminal R⁷ together with the carbon to which they are attached form a C₃-C₆ cycloalkyl or 3-9 membered heterocyclyl;
   each n is 0, 1, 2, 3, or 4;
   R⁸ is methyl, ethyl or cyano;
   each R^{A} is independently H, C₁-C₃ alkyl, C₃-C₆ cycloalkyl, or two geminal R^{A} together with the carbon to which they are attached form a C₃-C₆ cycloalkyl or 3-9 membered heterocyclyl; and
wherein each alkyl and cycloalkyl group is unsubstituted, substituted with 1-5 halo groups, perfluorinated, or is substituted by 1-3 groups selected from methyl, oxo, or carboxy.

In certain embodiments as otherwise described herein, for example in relation to compounds of Formulae (IIa), (IIb), and/or (IIc), Q is CH. In other embodiments, Q is N.

In certain embodiments as otherwise described herein, for example in relation to compounds of Formulae (IIa), (IIb), and/or (IIc), m is in the range of 1-3. For example, in particular embodiments, m is 2, and each R⁶ is methyl (e.g., unsubstituted methyl).

In certain embodiments as otherwise described herein, for example in relation to compounds of Formulae (IIa), (IIb), and/or (IIc), n is in the range of 1-3. For example, in particular embodiments, n is 1, and R⁷ is —COOH.

In one embodiment, the compound of Formula I is:

| No. | Structure | Name |
|---|---|---|
| 1 | 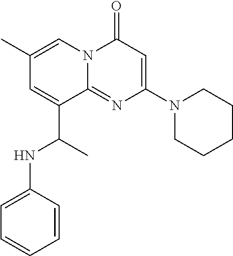 [SMILES: Cc1cc(C(C)Nc2ccccc2)c2nc(N3CCCCC3)cc(=O)n2c1] | 7-methyl-9-(1-(phenylamino)ethyl)-2- (piperidin-1-yl)-4H-pyrido[1,2-a]pyrimidin-4- one |
| 2 | 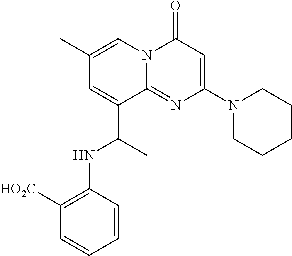 [SMILES: Cc1cc(C(C)Nc2ccccc2C(=O)O)c2nc(N3CCCCC3)cc(=O)n2c1] | 2-((1-(7-methyl-4-oxo-2-(piperidin-1-yl)-4H- pyrido[1,2-a]pyrimidin-9- yl)ethyl)amino)benzoic acid |
| 3 | 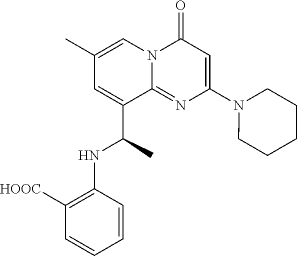 [SMILES: Cc1cc([C@@H](C)Nc2ccccc2C(=O)O)c2nc(N3CCCCC3)cc(=O)n2c1] | (R)-2-((1-(7-methyl-4-oxo-2-(piperidin-1-yl)- 4H-pyrido[1,2-a]pyrimidin-9- yl)ethyl)amino)benzoic acid |
| 4 | 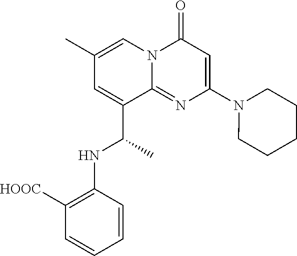 [SMILES: Cc1cc([C@H](C)Nc2ccccc2C(=O)O)c2nc(N3CCCCC3)cc(=O)n2c1] | (S)-2-((1-(7-methyl-4-oxo-2-(piperidin-1-yl)- 4H-pyrido[1,2-a]pyrimidin-9- yl)ethyl)amino)benzoic acid |
| 5 | 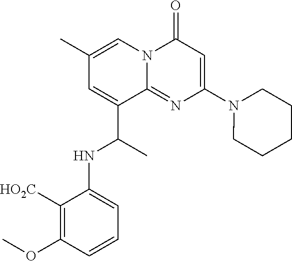 [SMILES: COc1cccc(NC(C)c2cc(C)cn3c(=O)cc(N4CCCCC4)nc23)c1C(=O)O] | 2-methoxy-6-((1-(7-methyl-4-oxo-2- (piperidin-1-yl)-4H-pyrido[1,2-a]pyrimidin-9- yl)ethyl)amino)benzoic acid |
| 6 | 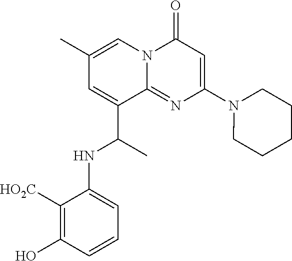 [SMILES: Cc1cc(C(C)Nc2cccc(O)c2C(=O)O)c2nc(N3CCCCC3)cc(=O)n2c1] | 2-hydroxy-6-((1-(7-methyl-4-oxo-2-(piperidin- 1-yl)-4H-pyrido[1,2-a]pyrimidin-9- yl)ethyl)amino)benzoic acid |
| 7 | 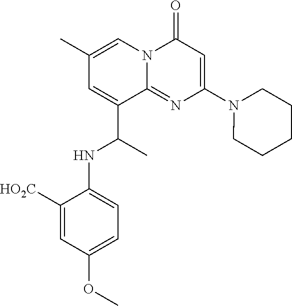 [SMILES: COc1ccc(NC(C)c2cc(C)cn3c(=O)cc(N4CCCCC4)nc23)c(C(=O)O)c1] | 5-methoxy-2-((1-(7-methyl-4-oxo-2- (piperidin-1-yl)-4H-pyrido[1,2-a]pyrimidin-9- yl)ethyl)amino)benzoic acid |
| 8 | 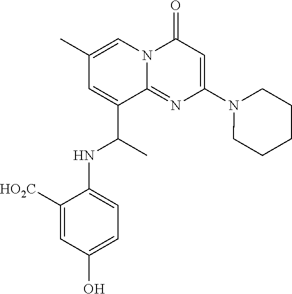 [SMILES: Cc1cc(C(C)Nc2ccc(O)cc2C(=O)O)c2nc(N3CCCCC3)cc(=O)n2c1] | 4-methoxy-2-((1-(7-methyl-4-oxo-2- (piperidin-1-yl)-4H-pyrido[1,2-a]pyrimidin-9- yl)ethyl)amino)butanoic acid |
| 9 | 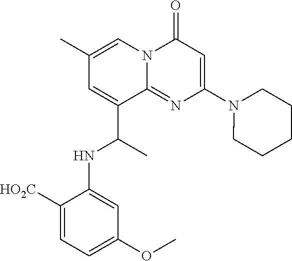 [SMILES: COc1ccc(C(=O)O)c(NC(C)c2cc(C)cn3c(=O)cc(N4CCCCC4)nc23)c1] | 4-hydroxy-2-((1-(7-methyl-4-oxo-2-(piperidin- 4-yl)-4H-pyrido[1,2-a]pyrimidin-9- yl)ethyl)amino)benzoic acid |
| 10 | 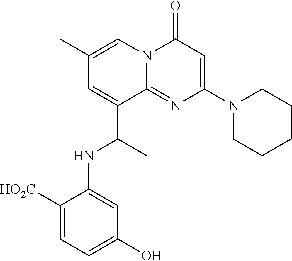 [SMILES: Cc1cc(C(C)Nc2cc(O)ccc2C(=O)O)c2nc(N3CCCCC3)cc(=O)n2c1] | 4-hydroxy-2-((1-(7-methyl-4-oxo-2-(piperidin- 1-yl)-4H-pyrido[1,2-a]pyrimidin-9- yl)ethyl)amino)benzoic acid |
| 11 | 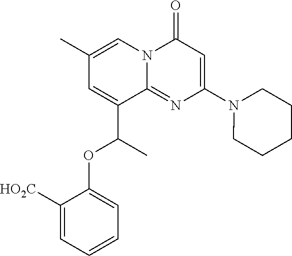 [SMILES: Cc1cc(C(C)Oc2ccccc2C(=O)O)c2nc(N3CCCCC3)cc(=O)n2c1] | 2-(1-(7-methyl-4-oxo-2-(piperidin-1-yl)-4H- pyrido[1,2-a]pyrimidin-9-yl)ethoxy)benzoic acid |
| 12 | 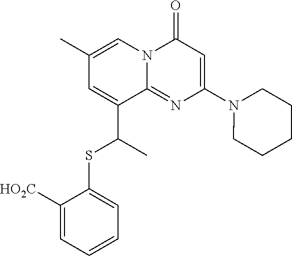 [SMILES: Cc1cc(C(C)Sc2ccccc2C(=O)O)c2nc(N3CCCCC3)cc(=O)n2c1] | 2-((1-(7-methyl-4-oxo-2-(piperidin-1-yl)-4H- pyrido[1,2-a]pyrimidin-9-yl)ethyl)thio)benzoic acid |
| 13 | 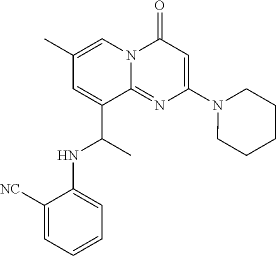 [SMILES: Cc1cc(C(C)Nc2ccccc2C#N)c2nc(N3CCCCC3)cc(=O)n2c1] | 2-((1-(7-methyl-4-oxo-2-(piperidin-1-yl)-4H- pyrido[1,2-a]pyrimidin-9- yl)ethyl)amino)benzonitrile |
| 14 | 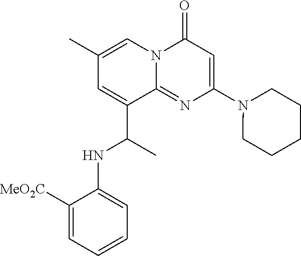 [SMILES: COC(=O)c1ccccc1NC(C)c1cc(C)cn2c(=O)cc(N3CCCCC3)nc12] | methyl 2-((1-(7-methyl-4-oxo-2-(piperidin-1- yl)-4H-pyrido[1,2-a]pyrimidin-9- yl)ethyl)amino)benzoate |
| 15 | 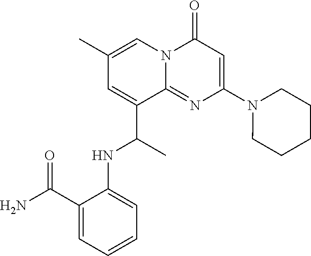 [SMILES: Cc1cc(C(C)Nc2ccccc2C(N)=O)c2nc(N3CCCCC3)cc(=O)n2c1] | 2-((1-(7-methyl-4-oxo-2-(piperidin-1-yl)-4H- pyrido[1,2-a]pyrimidin-9- yl)ethyl)amino)benzonitrile |
| 16 | 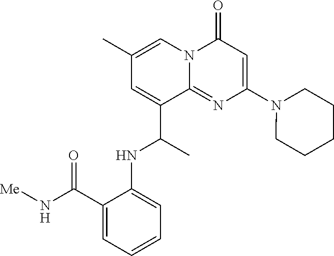 [SMILES: CNC(=O)c1ccccc1NC(C)c1cc(C)cn2c(=O)cc(N3CCCCC3)nc12] | N-methyl-2-((1-(7-methyl-4-oxo-2-(piperidin- 1-yl)-4H-pyrido[1,2-a]pyrimidin-9- yl)ethyl)amino)benzamide |
| 17 | 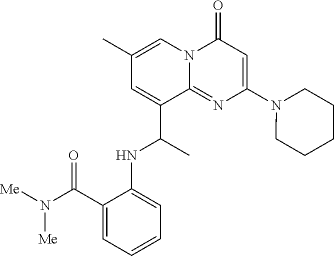 [SMILES: Cc1cc(C(C)Nc2ccccc2C(=O)N(C)C)c2nc(N3CCCCC3)cc(=O)n2c1] | N,N-dimethyl-2-((1-(7-methyl-4-oxo-2- (piperidin-1-yl)-4H-pyrido[1,2-a]pyrimidin-9- yl)ethyl)amino)benzamide |
| 18 | 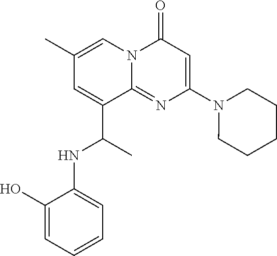 [SMILES: Cc1cc(C(C)Nc2ccccc2O)c2nc(N3CCCCC3)cc(=O)n2c1] | 9-(1-((2-hydroxyphenyl)amino)ethyl)-7- methyl-2-(piperidin-1-yl)-4H-pyrido[1,2- a]pyrimidin-4-one |
| 19 | 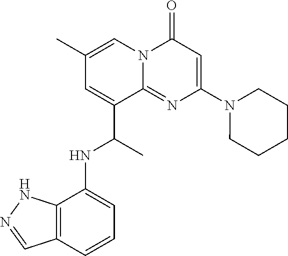 [SMILES: Cc1cc(C(C)Nc2cccc3cn[nH]c23)c2nc(N3CCCCC3)cc(=O)n2c1] | 9-(1-((1H-indazol-7-yl)amino)ethyl)-7-methyl- 2-(piperidin-1-yl)-4H-pyrido[1,2-a]pyrimidin- 4-one |
| 20 | 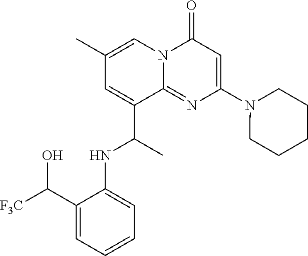 [SMILES: Cc1cc(C(C)Nc2ccccc2C(O)C(F)(F)F)c2nc(N3CCCCC3)cc(=O)n2c1] | 7-methyl-2-(piperidin-1-yl)-9-(1-((2-(2,2,2- trifluoro-1-hydroxyethyl)phenyl)amino)ethyl)- 4H-pyrido[1,2-a]pyrimidin-4-one |
| 21 | 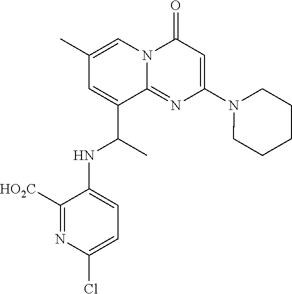 [SMILES: Cc1cc(C(C)Nc2ccc(Cl)nc2C(=O)O)c2nc(N3CCCCC3)cc(=O)n2c1] | 6-chloro-3-((1-(7-methyl-4-oxo-2-(piperidin- 1-yl)-4H-pyrido[1,2-a]pyrimidin-9- yl)ethyl)amino)picolinic acid |
| 22 | 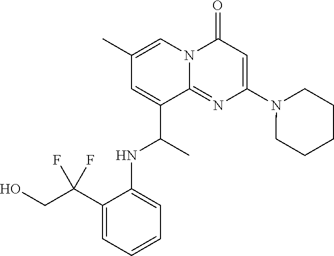 [SMILES: Cc1cc(C(C)Nc2ccccc2C(F)(F)CO)c2nc(N3CCCCC3)cc(=O)n2c1] | 9-(1-((2-(1,1-difluoro-2- hydroxyethyl)phenyl)amino)ethyl)-7-methyl- 2-(piperidin-1-yl)-4H-pyrido[1,2-a]pyrimidin-4-one |
| 23 | 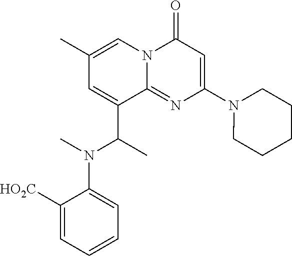 [SMILES: Cc1cc(C(C)N(C)c2ccccc2C(=O)O)c2nc(N3CCCCC3)cc(=O)n2c1] | 2-(methyl(1-(7-methyl-4-oxo-2-(piperidin-1- yl)-4H-pyrido[1,2-a]pyrimidin-9- yl)ethyl)amino)benzoic acid |
| 24 | 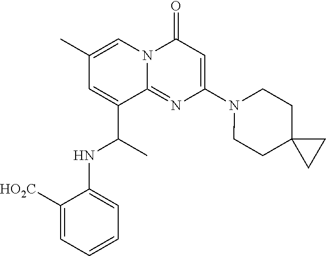 [SMILES: Cc1cc(C(C)Nc2ccccc2C(=O)O)c2nc(N3CCC4(CC3)CC4)cc(=O)n2c1] | 2-((1-(7-methyl-4-oxo-2-(6- azaspiro[2.5]octan-6-yl)- 4H-pyrido[1,2-a]pyrimidin-9-yl)ethyl)amino) benzoic acid |
| 25 | 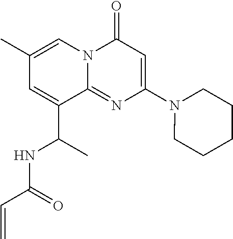 [SMILES: C=CC(=O)NC(C)c1cc(C)cn2c(=O)cc(N3CCCCC3)nc12] | N-(1-(7-methyl-4-oxo-2-(piperidin-1-yl)- 4H-pyrido[1,2-a]pyrimidin-9-yl)ethyl)acrylamide |
| 26 | 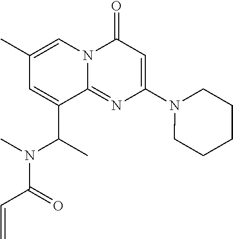 [SMILES: C=CC(=O)N(C)C(C)c1cc(C)cn2c(=O)cc(N3CCCCC3)nc12] | N-methyl-N-(1-(7-methyl-4-oxo-2-(piperidin- 1-yl)-4H-pyrido[1,2-a]pyrimidin-9- yl)ethyl)acrylamide |
| 27 | 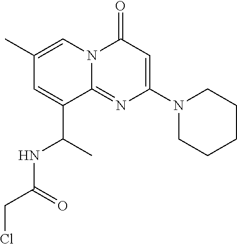 [SMILES: Cc1cc(C(C)NC(=O)CCl)c2nc(N3CCCCC3)cc(=O)n2c1] | 2-chloro-N-(1-(7-methyl-4-oxo-2-(piperidin-1- yl)-4H-pyrido[1,2-a]pyrimidin-9- yl)ethyl)acetamide |
| 28 | 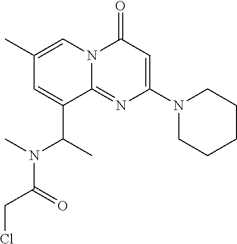 [SMILES: Cc1cc(C(C)N(C)C(=O)CCl)c2nc(N3CCCCC3)cc(=O)n2c1] | 2-chloro-N-methyl-N-(1-(7-methyl-4-oxo-2- (piperidin-1-yl)-4H-pyrido[1,2-a]pyrimidin-9- yl)ethyl)acetamide |
| 29 | 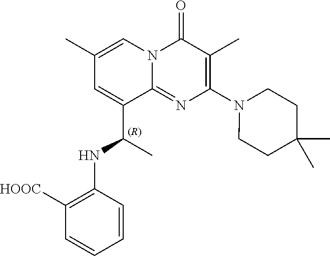 | (R)-2-((1-(2-(4,4-dimethylpiperidin-1-yl)-3,7- dimethyl-4-oxo-4H-pyrido[1,2-a]pyrimidin- 9-yl)ethyl)amino)benzoic acid |
| 30 | 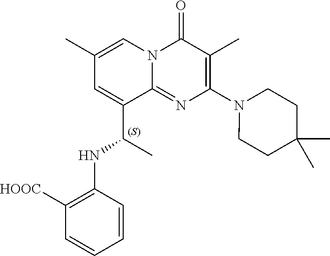 | (S)-2-((1-(2-(4,4-dimethylpiperidin-1-yl)-3,7- dimethyl-4-oxo-4H-pyrido[1,2-a]pyrimidin-9- yl)ethyl)amino)benzoic acid |
| 31 | 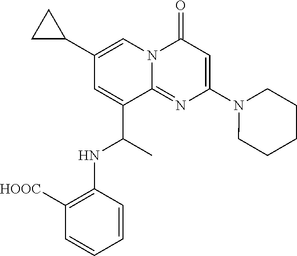 [SMILES: CC(Nc1ccccc1C(=O)O)c1cc(C2CC2)cn2c(=O)cc(N3CCCCC3)nc12] | 2-((1-(7-cyclopropyl-4-oxo-2-(piperidin-1-yl)- 4H-pyrido[1,2-a]pyrimidin-9- yl)ethyl)amino)benzoic acid |
| 32 | 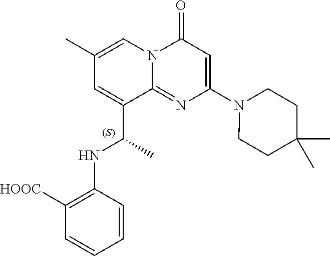 | (S)-2-((1-(2-(4,4-dimethylpiperidin-1-yl)-7- methyl-4-oxo-4H-pyrido[1,2-a]pyrimidin-9- yl)ethyl)amino)benzoic acid |
| 33 | 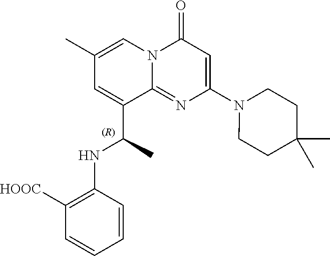 | (R)-2-((1-(2-(4,4-dimethylpiperidin-1-yl)-7- methyl-4-oxo-4H-pyrido[1,2-a]pyrimidin-9- yl)ethyl)amino)benzoic acid |
| 34 | 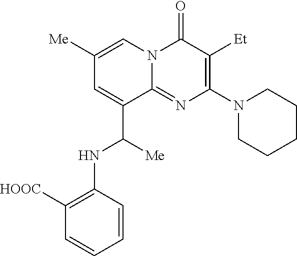 [SMILES: CCc1c(N2CCCCC2)nc2c(C(C)Nc3ccccc3C(=O)O)cc(C)cn2c1=O] | 2-((1-(3-ethyl-7-methyl-4-oxo-2-(piperidin-1- yl)-4H-pyrido[1,2-a]pyrimidin-9- yl)ethyl)amino)benzoic acid |
| 35 | 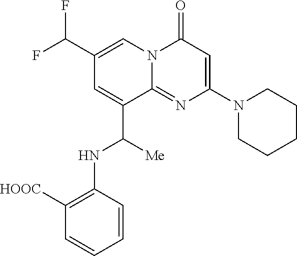 [SMILES: CC(Nc1ccccc1C(=O)O)c1cc(C(F)F)cn2c(=O)cc(N3CCCCC3)nc12] | 2-((1-(7-(difluoromethyl)-4-oxo-2-(piperidin- 1-yl)-4H-pyrido[1,2-a]pyrimidin-9- yl)ethyl)amino)benzoic acid |
| 36 | 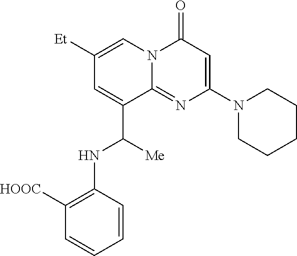 [SMILES: CCc1cc(C(C)Nc2ccccc2C(=O)O)c2nc(N3CCCCC3)cc(=O)n2c1] | 2-((1-(7-ethyl-4-oxo-2-(piperidin-1-yl)- 4H-pyrido[1,2-a]pyrimidin-9- yl)ethyl)amino)benzoic acid |
| 37 | 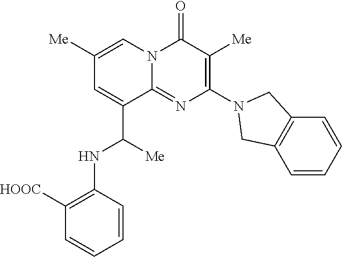 [SMILES: Cc1cc(C(C)Nc2ccccc2C(=O)O)c2nc(N3Cc4ccccc4C3)c(C)c(=O)n2c1] | 2-((1-(2-(isoindolin-2-yl)-3,7-dimethyl-4-oxo- 4H-pyrido[1,2-a]pyrimidin-9- yl)ethyl)amino)benzoic acid |
| 38 | 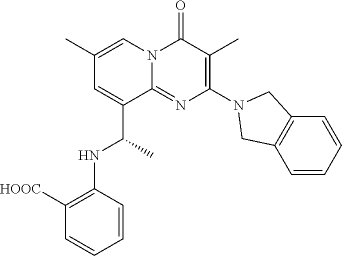 [SMILES: Cc1cc([C@H](C)Nc2ccccc2C(=O)O)c2nc(N3Cc4ccccc4C3)c(C)c(=O)n2c1] | (S)-2-((1-(2-(isoindolin-2-yl)-3,7-dimethyl-4- oxo-4H-pyrido[1,2-a]pyrimidin-9- yl)ethyl)amino)benzoic acid |
| 39 | 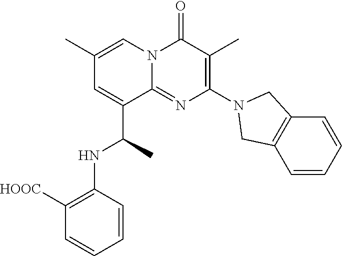 [SMILES: Cc1cc([C@@H](C)Nc2ccccc2C(=O)O)c2nc(N3Cc4ccccc4C3)c(C)c(=O)n2c1] | (R)-2-((1-(2-(isoindolin-2-yl)-3,7-dimethyl-4- oxo-4H-pyrido[1,2-a]pyrimidin-9- yl)ethyl)amino)benzoic acid |
| 40 | 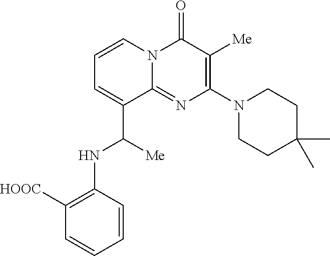 [SMILES: Cc1c(N2CCC(C)(C)CC2)nc2c(C(C)Nc3ccccc3C(=O)O)cccn2c1=O] | 2-((1-(2-(4,4-dimethylpiperidin-1-yl)-3- methyl-4-oxo-4H-pyrido[1,2-a]pyrimidin-9- yl)ethyl)amino)benzoic acid |
| 41 | 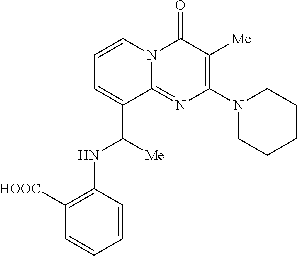 [SMILES: Cc1c(N2CCCCC2)nc2c(C(C)Nc3ccccc3C(=O)O)cccn2c1=O] | 2-((1-(3-methyl-4-oxo-2-(piperidin-1-yl)- 4H-pyrido[1,2-a]pyrimidin-9- yl)ethyl)amino)benzoic acid |
| 42 | 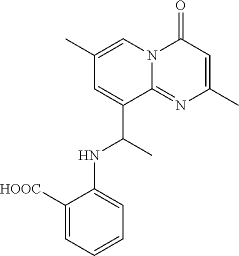 [SMILES: Cc1cc(C(C)Nc2ccccc2C(=O)O)c2nc(C)cc(=O)n2c1] | 2-((1-(2,7-dimethyl-4-oxo- 4H-pyrido[1,2-a]pyrimidin-9-yl)ethyl)amino) benzoic acid |
| 43 | 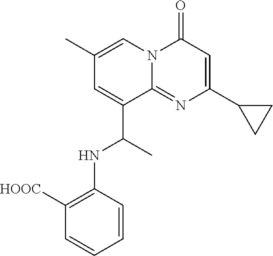 [SMILES: Cc1cc(C(C)Nc2ccccc2C(=O)O)c2nc(C3CC3)cc(=O)n2c1] | 2-((1-(2-cyclopropyl-7-methyl-4-oxo- 4H-pyrido[1,2-a]pyrimidin-9-yl) ethyl)amino)benzoic acid |
| 44 | 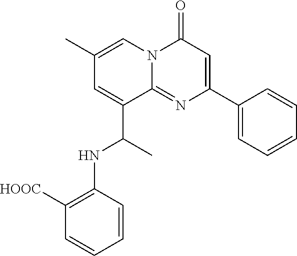 [SMILES: Cc1cc(C(C)Nc2ccccc2C(=O)O)c2nc(-c3ccccc3)cc(=O)n2c1] | 2-((1-(7-methyl-4-oxo-2-phenyl- 4H-pyrido[1,2-a]pyrimidin-9- yl)ethyl)amino)benzoic acid |
| 45 | 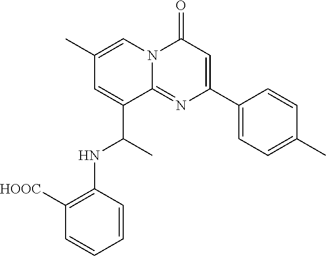 [SMILES: Cc1ccc(-c2cc(=O)n3cc(C)cc(C(C)Nc4ccccc4C(=O)O)c3n2)cc1] | 2-((1-(7-methyl-4-oxo-2-(p-tolyl)- 4H-pyrido[1,2-a]pyrimidin-9- yl)ethyl)amino)benzoic acid |
| 46 | 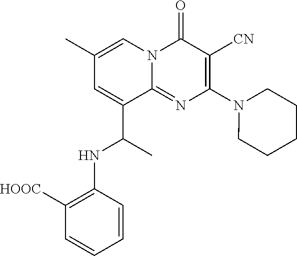 [SMILES: Cc1cc(C(C)Nc2ccccc2C(=O)O)c2nc(N3CCCCC3)c(C#N)c(=O)n2c1] | 2-((1-(3-cyano-7-methyl-4-oxo-2-(piperidin- 1-yl)-4H-pyrido[1,2-a]pyrimidin-9- yl)ethyl)amino)benzoic acid |
| 47 | 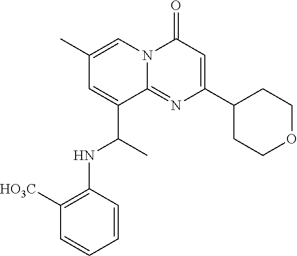 | 2-((1-(7-methyl-4-oxo-2-(tetrahydro-2H- pyran-4-yl)-4H-pyrido[1,2-a]pyrimidin-9- yl)ethyl)amino)benzoic acid |
| 48 | 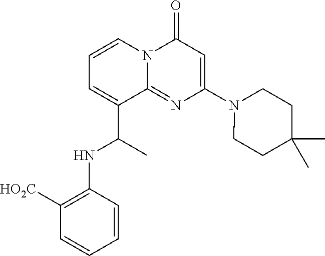 [SMILES: CC(Nc1ccccc1C(=O)O)c1cccn2c(=O)cc(N3CCC(C)(C)CC3)nc12] | 2-((1-(2-(4,4-dimethylpiperidin-1-yl)-4-oxo- 4H-pyrido[1,2-a]pyrimidin-9- yl)ethyl)amino)benzoic acid |
| 49 | 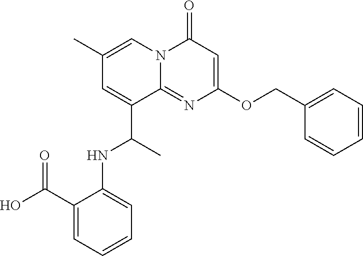 [SMILES: Cc1cc(C(C)Nc2ccccc2C(=O)O)c2nc(OCc3ccccc3)cc(=O)n2c1] | 2-((1-(2-(benzyloxy)-7-methyl-4-oxo- 4H-pyrido[1,2-a]pyrimidin-9- yl)ethyl)amino)benzoic acid |
| 50 | 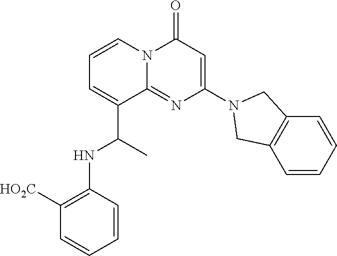 [SMILES: CC(Nc1ccccc1C(=O)O)c1cccn2c(=O)cc(N3Cc4ccccc4C3)nc12] | 2-((1-(2-(isoindolin-2-yl)-4-oxo- 4H-pyrido[1,2-a]pyrimidin-9- yl)ethyl)amino)benzoic acid |
| 51 | 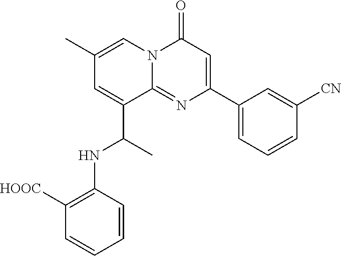 [SMILES: Cc1cc(C(C)Nc2ccccc2C(=O)O)c2nc(-c3cccc(C#N)c3)cc(=O)n2c1] | 2-((1-(2-(3-cyanophenyl)-7-methyl-4-oxo- 4H-pyrido[1,2-a]pyrimidin-9- yl)ethyl)amino)benzoic acid |
| 52 | 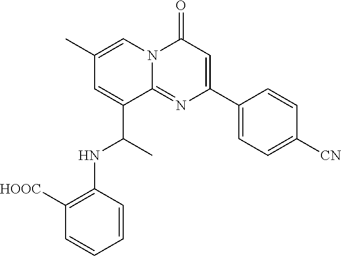 [SMILES: Cc1cc(C(C)Nc2ccccc2C(=O)O)c2nc(-c3ccc(C#N)cc3)cc(=O)n2c1] | 2-((1-(2-(4-cyanophenyl)-7-methyl-4-oxo- 4H-pyrido[1,2-a]pyrimidin-9- yl)ethyl)amino)benzoic acid |
| 53 | 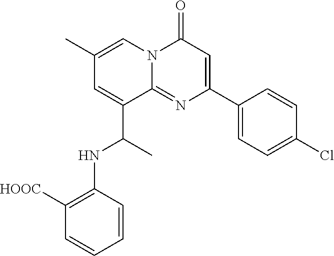 [SMILES: Cc1cc(C(C)Nc2ccccc2C(=O)O)c2nc(-c3ccc(Cl)cc3)cc(=O)n2c1] | 2-((1-(2-(4-chlorophenyl)-7-methyl-4-oxo- 4H-pyrido[1,2-a]pyrimidin-9- yl)ethyl)amino)benzoic acid |
| 54 | 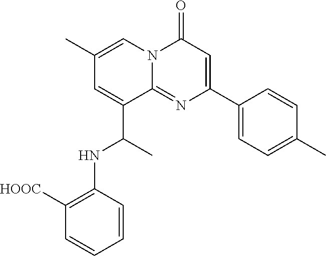 [SMILES: Cc1ccc(-c2cc(=O)n3cc(C)cc(C(C)Nc4ccccc4C(=O)O)c3n2)cc1] | 2-((1-(7-methyl-4-oxo-2-(p-tolyl)- 4H-pyrido[1,2-a]pyrimidin-9- yl)ethyl)amino)benzoic acid |
| 55 | 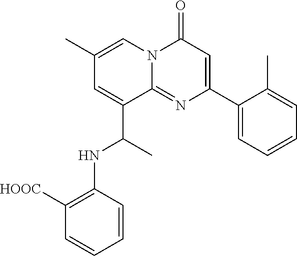 [SMILES: Cc1cc(C(C)Nc2ccccc2C(=O)O)c2nc(-c3ccccc3C)cc(=O)n2c1] | 2-((1-(7-methyl-4-oxo-2-(o-tolyl)- 4H-pyrido[1,2-a]pyrimidin-9- yl)ethyl)amino)benzoic acid |
| 56 | 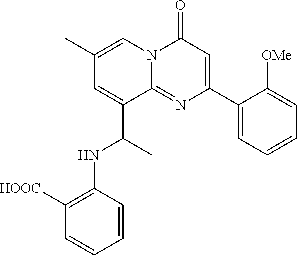 [SMILES: COc1ccccc1-c1cc(=O)n2cc(C)cc(C(C)Nc3ccccc3C(=O)O)c2n1] | 2-((1-(2-(2-methoxyphenyl)-7-methyl-4-oxo- 4H-pyrido[1,2-a]pyrimidin-9- yl)ethyl)amino)benzoic acid |
| 57 | 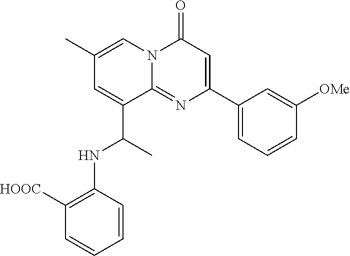 [SMILES: COc1cccc(-c2cc(=O)n3cc(C)cc(C(C)Nc4ccccc4C(=O)O)c3n2)c1] | 2-((1-(2-(3-methoxyphenyl)-7-methyl-4-oxo- 4H-pyrido[1,2-a]pyrimidin-9- yl)ethyl)amino)benzoic acid |
| 58 | 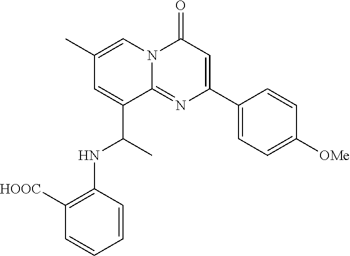 [SMILES: COc1ccc(-c2cc(=O)n3cc(C)cc(C(C)Nc4ccccc4C(=O)O)c3n2)cc1] | 2-((1-(2-(4-methoxyphenyl)-7-methyl-4-oxo- 4H-pyrido[1,2-a]pyrimidin-9- yl)ethyl)amino)benzoic acid |
| 59 | 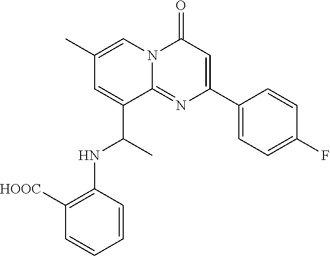 [SMILES: Cc1cc(C(C)Nc2ccccc2C(=O)O)c2nc(-c3ccc(F)cc3)cc(=O)n2c1] | 2-((1-(2-(4-fluorophenyl)-7-methyl-4-oxo- 4H-pyrido[1,2-a]pyrimidin-9- yl)ethyl)amino)benzoic acid |
| 60 | 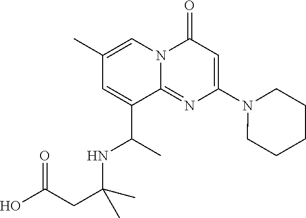 [SMILES: Cc1cc(C(C)NC(C)(C)CC(=O)O)c2nc(N3CCCCC3)cc(=O)n2c1] | 3-methyl-3-((1-(7-methyl-4-oxo-2-(piperidin- 1-yl)-4H-pyrido[1,2-a]pyrimidin-9- yl)ethyl)amino)butanoic acid |
| 61 | 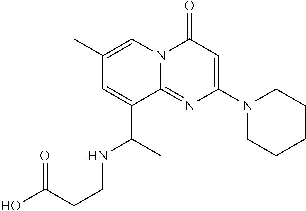 [SMILES: Cc1cc(C(C)NCCC(=O)O)c2nc(N3CCCCC3)cc(=O)n2c1] | 3-((1-(7-methyl-4-oxo-2-(piperidin-1-yl)- 4H-pyrido[1,2-a]pyrimidin-9- yl)ethyl)amino)propanoic acid |
| 62 | 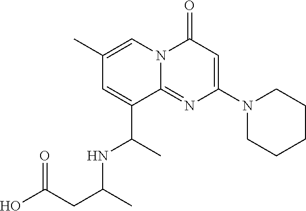 [SMILES: Cc1cc(C(C)NC(C)CC(=O)O)c2nc(N3CCCCC3)cc(=O)n2c1] | 3-((1-(7-methyl-4-oxo-2-(piperidin-1-yl)- 4H-pyrido[1,2-a]pyrimidin-9- yl)ethyl)amino)butanoic acid |
| 63 | 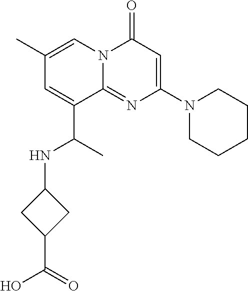 [SMILES: Cc1cc(C(C)NC2CC(C(=O)O)C2)c2nc(N3CCCCC3)cc(=O)n2c1] | 3-((1-(7-methyl-4-oxo-2-(piperidin-1-yl)- 4H-pyrido[1,2-a]pyrimidin-9- yl)ethyl)amino)cyclobutane-1-carboxylic acid |
| 64 | 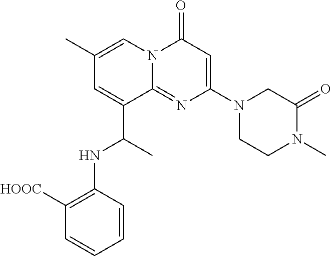 [SMILES: Cc1cc(C(C)Nc2ccccc2C(=O)O)c2nc(N3CCN(C)C(=O)C3)cc(=O)n2c1] | 2-((1-(7-methyl-2-(4-methyl-3-oxopiperazin- 1-yl)-4-oxo-4H-pyrido[1,2-a]pyrimidin-9- yl)ethyl)amino)benzoic acid |
| 65 | 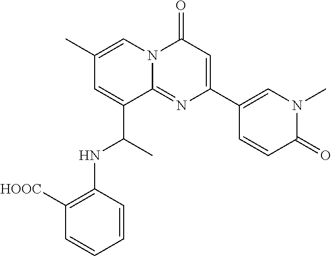 [SMILES: Cc1cc(C(C)Nc2ccccc2C(=O)O)c2nc(-c3ccc(=O)n(C)c3)cc(=O)n2c1] | 2-((1-(7-methyl-2-(1-methyl-6-oxo-1,6- dihydropyridin-3-yl)-4-oxo- 4H-pyrido[1,2-a]pyrimidin-9-yl)ethyl)amino) benzoic acid |
| 66 | 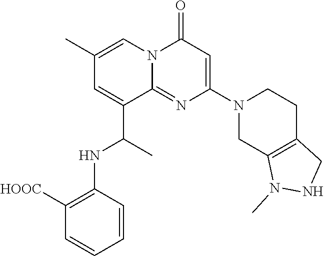 [SMILES: Cc1cc(C(C)Nc2ccccc2C(=O)O)c2nc(N3CCC4=C(C3)N(C)NC4)cc(=O)n2c1] | 2-((1-(7-methyl-2-(1-methyl-1,2,3,4,5,7- hexahydro-6H-pyrazolo[3,4-c]pyridin-6-yl)-4- oxo-4H-pyrido[1,2-a]pyrimidin-9- yl)ethyl)amino)benzoic acid |
| 67 | 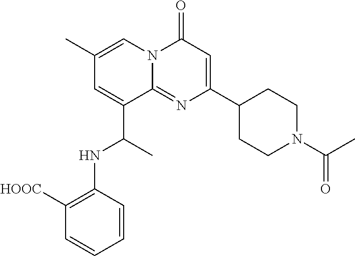 [SMILES: CC(=O)N1CCC(c2cc(=O)n3cc(C)cc(C(C)Nc4ccccc4C(=O)O)c3n2)CC1] | 2-((1-(2-(1-acetylpiperidin-4-yl)-7-methyl-4- oxo-4H-pyrido[1,2-a]pyrimidin-9- yl)ethyl)amino)benzoic acid |
| 68 | 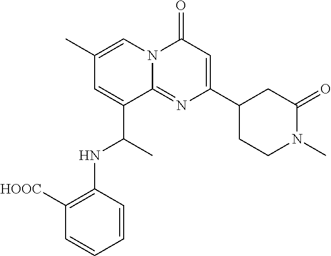 [SMILES: Cc1cc(C(C)Nc2ccccc2C(=O)O)c2nc(C3CCN(C)C(=O)C3)cc(=O)n2c1] | 2-((1-(7-methyl-2-(1-methyl-2-oxopiperidin-4- yl)-4-oxo-4H-pyrido[1,2-a]pyrimidin-9- yl)ethyl)amino)benzoic acid |
| 69 | 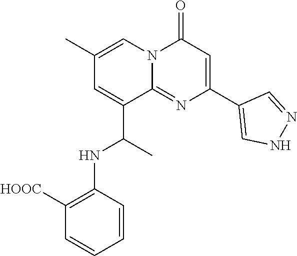 [SMILES: Cc1cc(C(C)Nc2ccccc2C(=O)O)c2nc(-c3cn[nH]c3)cc(=O)n2c1] | 2-((1-(7-methyl-4-oxo-2-(1H-pyrazol-4-yl)- 4H-pyrido[1,2-a]pyrimidin-9- yl)ethyl)amino)benzoic acid |
| 70 | 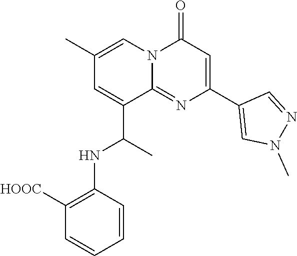 [SMILES: Cc1cc(C(C)Nc2ccccc2C(=O)O)c2nc(-c3cnn(C)c3)cc(=O)n2c1] | 2-((1-(7-methyl-2-(1-methyl-1H-pyrazol-4-yl)- 4-oxo-4H-pyrido[1,2-a]pyrimidin-9- yl)ethyl)amino)benzoic acid |
| 71 | 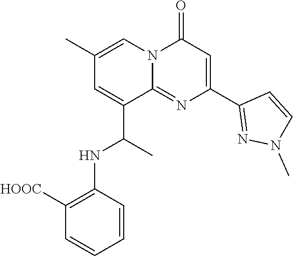 [SMILES: Cc1cc(C(C)Nc2ccccc2C(=O)O)c2nc(-c3ccn(C)n3)cc(=O)n2c1] | 2-((1-(7-methyl-2-(1-methyl-1H-pyrazol-3-yl)- 4-oxo-4H-pyrido[1,2-a]pyrimidin-9- yl)ethyl)amino)benzoic acid |
| 72 | 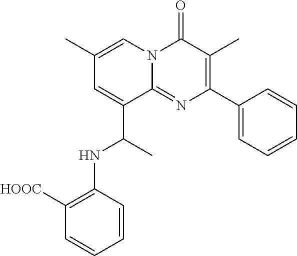 [SMILES: Cc1cc(C(C)Nc2ccccc2C(=O)O)c2nc(-c3ccccc3)c(C)c(=O)n2c1] | 2-((1-(3,7-dimethyl-4-oxo-2-phenyl- 4H-pyrido[1,2-a]pyrimidin-9- yl)ethyl)amino)benzoic acid |
| 73 | 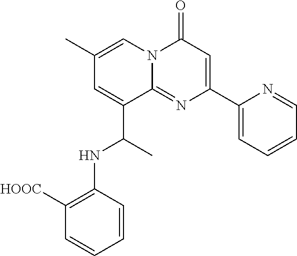 [SMILES: Cc1cc(C(C)Nc2ccccc2C(=O)O)c2nc(-c3ccccn3)cc(=O)n2c1] | 2-((1-(7-methyl-4-oxo-2-(pyridin-2-yl)- 4H-pyrido[1,2-a]pyrimidin-9- yl)ethyl)amino)benzoic acid |
| 74 | 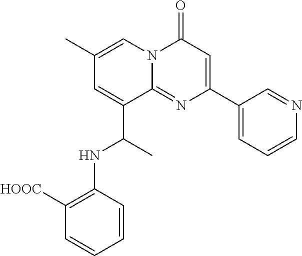 [SMILES: Cc1cc(C(C)Nc2ccccc2C(=O)O)c2nc(-c3cccnc3)cc(=O)n2c1] | 2-((1-(7-methyl-4-oxo-2-(pyridin-3-yl)- 4H-pyrido[1,2-a]pyrimidin-9- yl)ethyl)amino)benzoic acid |
| 75 | 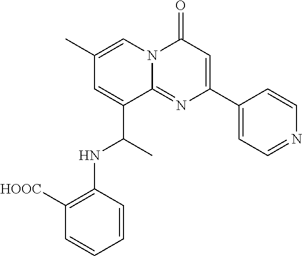 [SMILES: Cc1cc(C(C)Nc2ccccc2C(=O)O)c2nc(-c3ccncc3)cc(=O)n2c1] | 2-((1-(7-methyl-4-oxo-2-(pyridin-4-yl)- 4H-pyrido[1,2-a]pyrimidin-9- yl)ethyl)amino)benzoic acid |
| 76 | 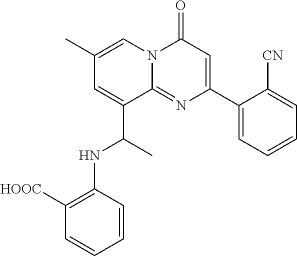 [SMILES: Cc1cc(C(C)Nc2ccccc2C(=O)O)c2nc(-c3ccccc3C#N)cc(=O)n2c1] | 2-((1-(2-(2-cyanophenyl)-7-methyl-4-oxo-4H- pyrido[1,2-a]pyrimidin-9- yl)ethyl)amino)benzoic acid |
| 77 | 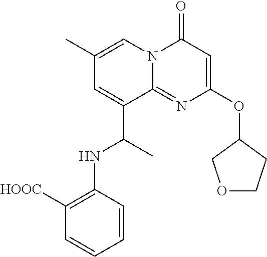 [SMILES: Cc1cc(C(C)Nc2ccccc2C(=O)O)c2nc(OC3CCOC3)cc(=O)n2c1] | 2-((1-(7-methyl-4-oxo-2-((tetrahydrofuran-3- yl)oxy)-4H-pyrido[1,2-a]pyrimidin-9- yl)ethyl)amino)benzoic acid |

or a pharmaceutically acceptable salt of one of the foregoing compounds.

In another embodiment, the compound of Formula I is selected from:

| No. | Structure | Name |
|---|---|---|
| 10-1 | 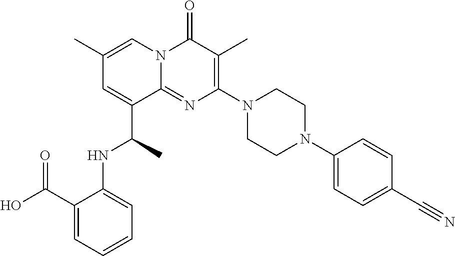 [SMILES: Cc1cc([C@@H](C)Nc2ccccc2C(=O)O)c2nc(N3CCN(c4ccc(C#N)cc4)CC3)c(C)c(=O)n2c1] | (R)-2-((1-(2-(4-(4- cyanophenyl)piperazin-1-yl)-3,7- dimethyl-4-oxo-4H-pyrido[1,2- a]pyrimidin-9- yl)ethyl)amino)benzoic acid |
| 10-2 | 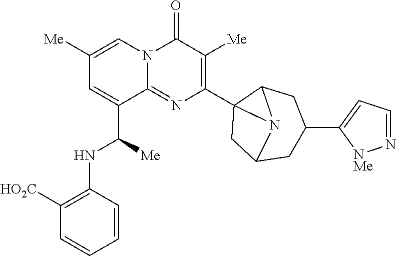 [SMILES: Cc1cc([C@@H](C)Nc2ccccc2C(=O)O)c2nc(C34CC5CC(c6ccnn6C)CC3N54)c(C)c(=O)n2c1] | 2-(((1R)-1-(3,7-dimethyl-2- (3-(1-methyl-1H-pyrazol-5-yl)-8- azabicyclo[3.2.1]octan-8-yl)-4- oxo-4H-pyrido[1,2-a]pyrimidin-9- yl)ethyl)amino)benzoic acid |
| 10-3 | 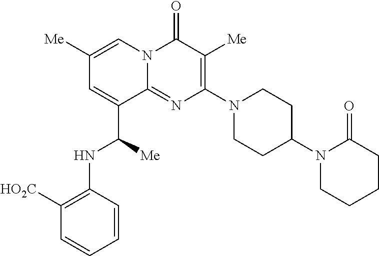 [SMILES: Cc1cc([C@@H](C)Nc2ccccc2C(=O)O)c2nc(N3CCC(N4CCCCC4=O)CC3)c(C)c(=O)n2c1] | (R)-2-((1-(3,7-dimethyl-4- oxo-2-(2-oxo-[1,4′-bipiperidin]-1′- yl)-4H-pyrido[1,2-a]pyrimidin-9- yl)ethyl)amino)benzoic acid |
| 10-4 | 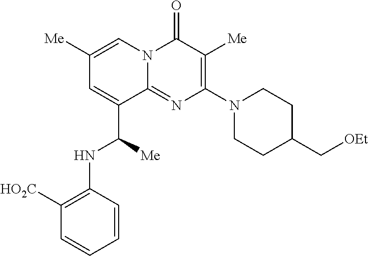 [SMILES: CCOCC1CCN(c2nc3c([C@@H](C)Nc4ccccc4C(=O)O)cc(C)cn3c(=O)c2C)CC1] | (R)-2-((1-(2-(4- (ethoxymethyl)piperidin-1-yl)-3,7- dimethyl-4-oxo-4H-pyrido[1,2- a]pyrimidin-9- yl)ethyl)amino)benzoic acid |
| 10-5 | 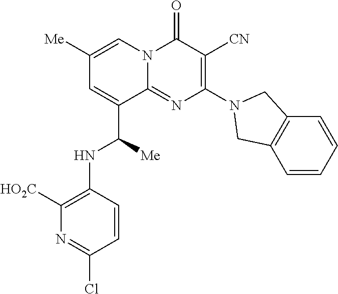 [SMILES: Cc1cc([C@@H](C)Nc2ccc(Cl)nc2C(=O)O)c2nc(N3Cc4ccccc4C3)c(C#N)c(=O)n2c1] | (R)-3-((1-(3-cyano-2- (isoindolin-2-yl)-7-methyl-4-oxo- 4H-pyrido[1,2-a]pyrimidin-9- yl)ethyl)amino)-6-methylpicolinic acid |
| 10-6 | 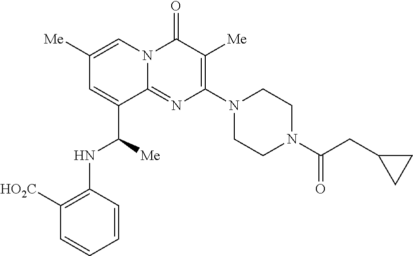 [SMILES: Cc1cc([C@@H](C)Nc2ccccc2C(=O)O)c2nc(N3CCN(C(=O)CC4CC4)CC3)c(C)c(=O)n2c1] | (R)-2-((1-(2-(4-(2- cyclopropylacetyl)piperazin-1-yl)- 3,7-dimethyl-4-oxo-4H-pyrido[1,2- a]pyrimidin-9- yl)ethyl)amino)benzoic acid |
| 10-7 | 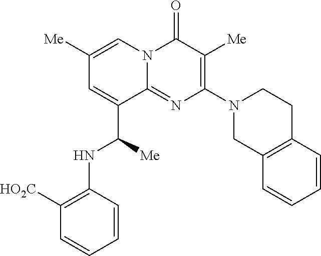 [SMILES: Cc1cc([C@@H](C)Nc2ccccc2C(=O)O)c2nc(N3CCc4ccccc4C3)c(C)c(=O)n2c1] | (R)-2-((1-(2-(3,4- dihydroisoquinolin-2(1H)-yl)-3,7- dimethyl-4-oxo-4H-pyrido[1,2- a]pyrimidin-9- yl)ethyl)amino)benzoic acid |
| 10-8 | 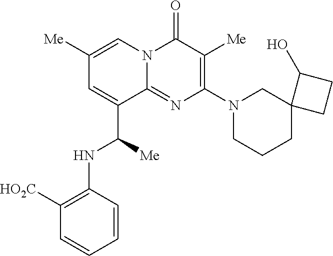 [SMILES: Cc1cc([C@@H](C)Nc2ccccc2C(=O)O)c2nc(N3CCCC4(CCC4O)C3)c(C)c(=O)n2c1] | 2-(((1R)-1-(2-(1-hydroxy-6- azaspiro[3.5]nonan-6-yl)-3,7- dimethyl-4-oxo-4H-pyrido[1,2- a]pyrimidin-9- yl)ethyl)amino)benzoic acid |
| 10-9 | 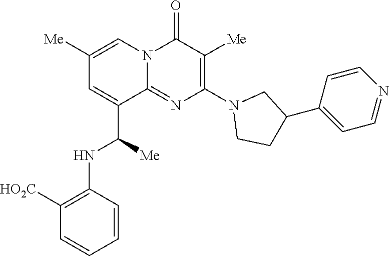 [SMILES: Cc1cc([C@@H](C)Nc2ccccc2C(=O)O)c2nc(N3CCC(c4ccncc4)C3)c(C)c(=O)n2c1] | 2-(((1R)-1-(3,7-dimethyl-4- oxo-2-(3-(pyridin-4-yl)pyrrolidin-1- yl)-4H-pyrido[1,2-a]pyrimidin-9- yl)ethyl)amino)benzoic acid |
| 10-10 | 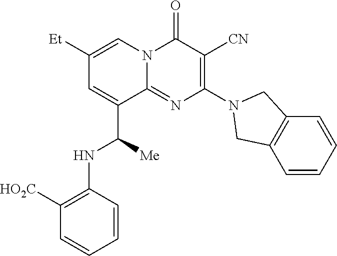 [SMILES: CCc1cc([C@@H](C)Nc2ccccc2C(=O)O)c2nc(N3Cc4ccccc4C3)c(C#N)c(=O)n2c1] | (R)-2-((1-(3-cyano-7-ethyl- 2-(isoindolin-2-yl)-4-oxo-4H- pyrido[1,2-a]pyrimidin-9- yl)ethyl)amino)benzoic acid |
| 10-11 | 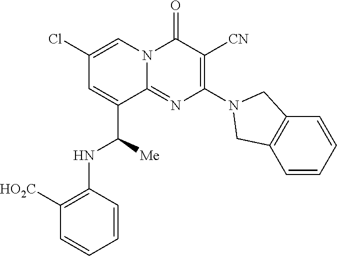 [SMILES: C[C@@H](Nc1ccccc1C(=O)O)c1cc(Cl)cn2c(=O)c(C#N)c(N3Cc4ccccc4C3)nc12] | (R)-2-((1-(7-chloro-3-cyano- 2-(isoindolin-2-yl)-4-oxo-4H- pyrido[1,2-a]pyrimidin-9- yl)ethyl)amino)benzoic acid |
| 10-12 | 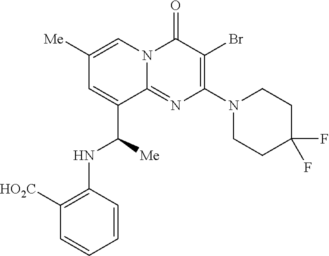 [SMILES: Cc1cc([C@@H](C)Nc2ccccc2C(=O)O)c2nc(N3CCC(F)(F)CC3)c(Br)c(=O)n2c1] | (R)-2-((1-(3-bromo-2-(4,4- difluoropiperidin-1-yl)-7-methyl-4- oxo-4H-pyrido[1,2-a]pyrimidin-9- yl)ethyl)amino)benzoic acid |
| 10-13 | 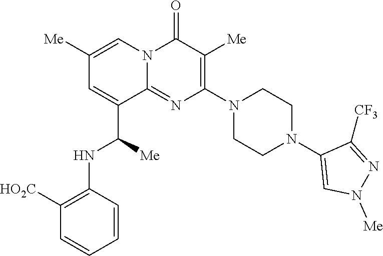 [SMILES: Cc1cc([C@@H](C)Nc2ccccc2C(=O)O)c2nc(N3CCN(c4cn(C)nc4C(F)(F)F)CC3)c(C)c(=O)n2c1] | (R)-2-((1-(3,7-dimethyl-2-(4- (1-methyl-3-(trifluoromethyl)-1H- pyrazol-4-yl)piperazin-1-yl)-4-oxo- 4H-pyrido[1,2-a]pyrimidin-9- yl)ethyl)amino)benzoic acid |
| 10-14 | 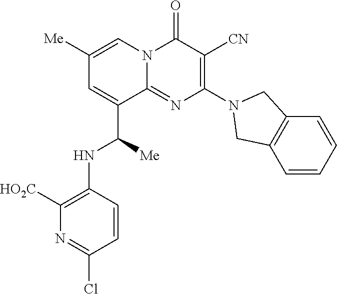 [SMILES: Cc1cc([C@@H](C)Nc2ccc(Cl)nc2C(=O)O)c2nc(N3Cc4ccccc4C3)c(C#N)c(=O)n2c1] | (R)-6-chloro-3-((1-(3-cyano- 2-(isoindolin-2-yl)-7-methyl-4-oxo- 4H-pyrido[1,2-a]pyrimidin-9- yl)ethyl)amino)picolinic acid |
| 10-15 | 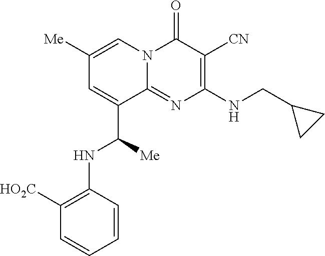 [SMILES: Cc1cc([C@@H](C)Nc2ccccc2C(=O)O)c2nc(NCC3CC3)c(C#N)c(=O)n2c1] | (R)-2-((1-(3-cyano-2- ((cyclopropylmethyl)amino)-7- methyl-4-oxo-4H-pyrido[1,2- a]pyrimidin-9- yl)ethyl)amino)benzoic acid |
| 10-16 | 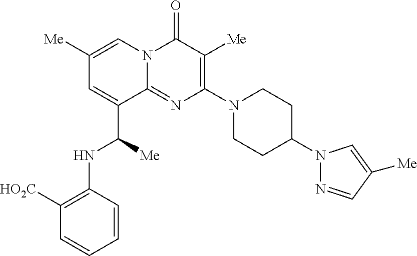 [SMILES: Cc1cnn(C2CCN(c3nc4c([C@@H](C)Nc5ccccc5C(=O)O)cc(C)cn4c(=O)c3C)CC2)c1] | (R)-2-((1-(3,7-dimethyl-2-(4- (4-methyl-1H-pyrazol-1- yl)piperidin-1-yl)-4-oxo-4H- pyrido[1,2-a]pyrimidin-9- yl)ethyl)amino)benzoic acid |
| 10-17 | 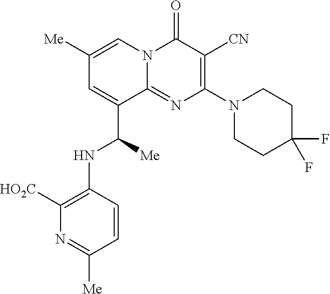 [SMILES: Cc1cc([C@@H](C)Nc2ccc(C)nc2C(=O)O)c2nc(N3CCC(F)(F)CC3)c(C#N)c(=O)n2c1] | (R)-3-((1-(3-cyano-2-(4,4- difluoropiperidin-1-yl)-7-methyl-4- oxo-4H-pyrido[1,2-a]pyrimidin-9- yl)ethyl)amino)-6-methylpicolinic acid |
| 10-18 | 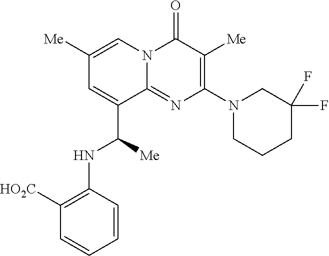 [SMILES: Cc1cc([C@@H](C)Nc2ccccc2C(=O)O)c2nc(N3CCCC(F)(F)C3)c(C)c(=O)n2c1] | (R)-2-((1-(2-(3,3- difluoropiperidin-1-yl)-3,7- dimethyl-4-oxo-4H-pyrido[1,2- a]pyrimidin-9- yl)ethyl)amino)benzoic acid |
| 10-19 | 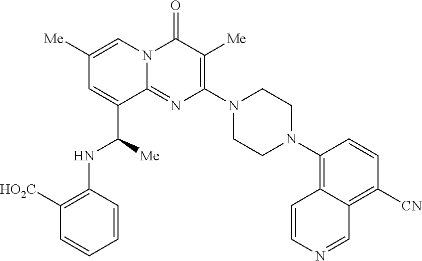 [SMILES: Cc1cc([C@@H](C)Nc2ccccc2C(=O)O)c2nc(N3CCN(c4ccc(C#N)c5cnccc45)CC3)c(C)c(=O)n2c1] | (R)-2-((1-(2-(4-(8- cyanoisoquinolin-5-yl)piperazin-1- yl)-3,7-dimethyl-4-oxo-4H- pyrido[1,2-a]pyrimidin-9- yl)ethyl)amino)benzoic acid |
| 10-20 | 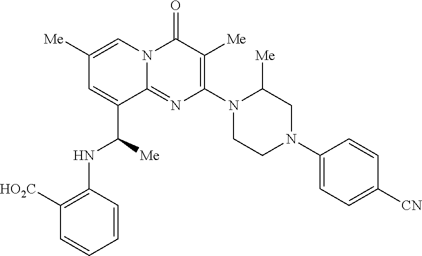 [SMILES: Cc1cc([C@@H](C)Nc2ccccc2C(=O)O)c2nc(N3CCN(c4ccc(C#N)cc4)CC3C)c(C)c(=O)n2c1] | 2-(((1R)-1-(2-(4-(4- cyanophenyl)-2-methylpiperazin- 1-yl)-3,7-dimethyl-4-oxo-4H- pyrido[1,2-a]pyrimidin-9- yl)ethyl)amino)benzoic acid |
| 10-21 | 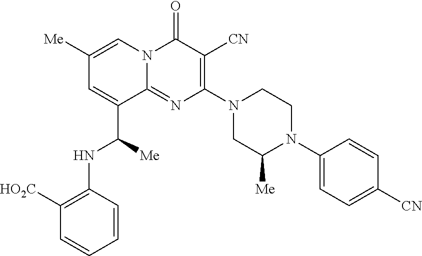 [SMILES: Cc1cc([C@@H](C)Nc2ccccc2C(=O)O)c2nc(N3CCN(c4ccc(C#N)cc4)[C@@H](C)C3)c(C#N)c(=O)n2c1] | 2-(((R)-1-(2-((S)-4-(4- cyanophenyl)-3-methylpiperazin- 1-yl)-3,7-dimethyl-4-oxo-4H- pyrido[1,2-a]pyrimidin-9- yl)ethyl)amino)benzoic acid |
| 10-22 | 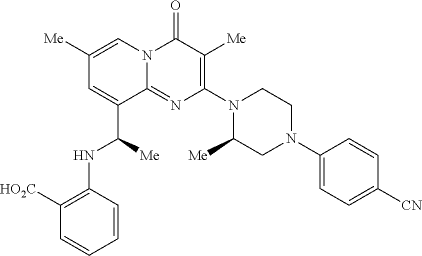 [SMILES: Cc1cc([C@@H](C)Nc2ccccc2C(=O)O)c2nc(N3CCN(c4ccc(C#N)cc4)C[C@H]3C)c(C)c(=O)n2c1] | 2-(((R)-1-(2-((R)-4-(4- cyanophenyl)-2-methylpiperazin- 1-yl)-3,7-dimethyl-4-oxo-4H- pyrido[1,2-a]pyrimidin-9- yl)ethyl)amino)benzoic acid |
| 10-23 | 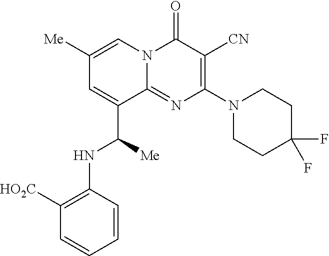 [SMILES: Cc1cc([C@@H](C)Nc2ccccc2C(=O)O)c2nc(N3CCC(F)(F)CC3)c(C#N)c(=O)n2c1] | (R)-2-((1-(3-cyano-2-(4,4- difluoropiperidin-1-yl)-7-methyl-4- oxo-4H-pyrido[1,2-a]pyrimidin-9- yl)ethyl)amino)benzoic acid |
| 10-24 | 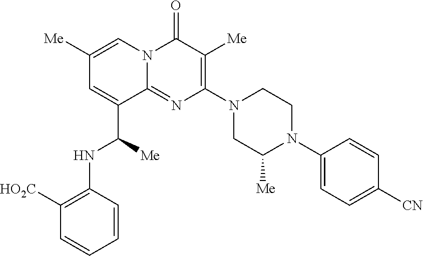 [SMILES: Cc1cc([C@@H](C)Nc2ccccc2C(=O)O)c2nc(N3CCN(c4ccc(C#N)cc4)[C@H](C)C3)c(C)c(=O)n2c1] | 2-(((R)-1-(2-((R)-4-(4- cyanophenyl)-3-methylpiperazin- 1-yl)-3,7-dimethyl-4-oxo-4H- pyrido[1,2-a]pyrimidin-9- yl)ethyl)amino)benzoic acid |
| 10-25 | 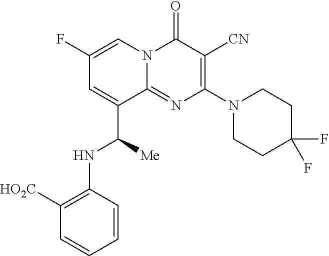 [SMILES: C[C@@H](Nc1ccccc1C(=O)O)c1cc(F)cn2c(=O)c(C#N)c(N3CCC(F)(F)CC3)nc12] | (R)-2-((1-(3-cyano-2-(4,4- difluoropiperidin-1-yl)-7-fluoro-4- oxo-4H-pyrido[1,2-a]pyrimidin-9- yl)ethyl)amino)benzoic acid |
| 10-26 | 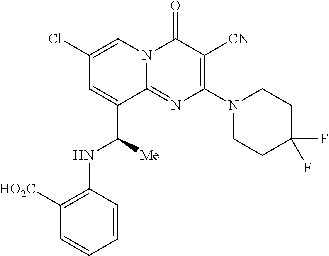 [SMILES: C[C@@H](Nc1ccccc1C(=O)O)c1cc(Cl)cn2c(=O)c(C#N)c(N3CCC(F)(F)CC3)nc12] | (R)-2-((1-(7-chloro-3-cyano- 2-(4,4-difluoropiperidin-1-yl)-4- oxo-4H-pyrido[1,2-a]pyrimidin-9- yl)ethyl)amino)benzoic acid |
| 10-27 | 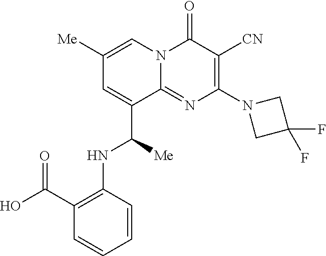 [SMILES: Cc1cc([C@@H](C)Nc2ccccc2C(=O)O)c2nc(N3CC(F)(F)C3)c(C#N)c(=O)n2c1] | (R)-2-((1-(3-cyano-2-(3,3- difluoroazetidin-1-yl)-7-methyl-4- oxo-4H-pyrido[1,2-a]pyrimidin-9- yl)ethyl)amino)benzoic acid |
| 10-28 | 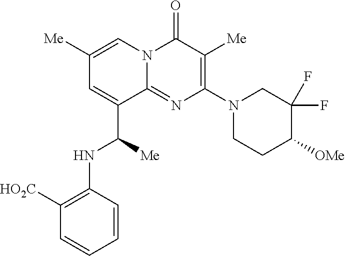 [SMILES: CO[C@@H]1CCN(c2nc3c([C@@H](C)Nc4ccccc4C(=O)O)cc(C)cn3c(=O)c2C)CC1(F)F] | 2-(((R)-1-(2-((R)-3,3- difluoro-4-methoxypiperidin-1-yl)- 3,7-dimethyl-4-oxo-4H-pyrido[1,2- a]pyrimidin-9- yl)ethyl)amino)benzoic acid |
| 10-29 | 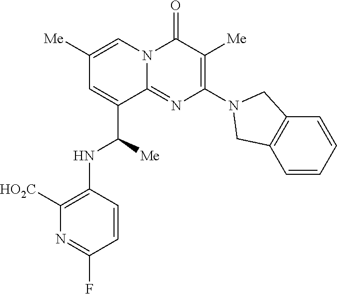 [SMILES: Cc1cc([C@@H](C)Nc2ccc(F)nc2C(=O)O)c2nc(N3Cc4ccccc4C3)c(C)c(=O)n2c1] | (R)-6-fluoro-3-((1-(2- (isoindolin-2-yl)-3,7-dimethyl-4- oxo-4H-pyrido[1,2-a]pyrimidin-9- yl)ethyl)amino)picolinic acid |
| 10-30 | 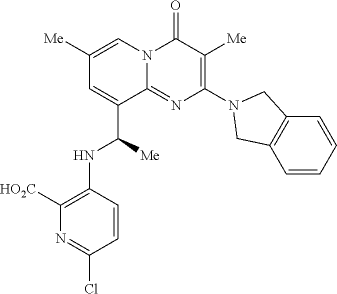 [SMILES: Cc1cc([C@@H](C)Nc2ccc(Cl)nc2C(=O)O)c2nc(N3Cc4ccccc4C3)c(C)c(=O)n2c1] | (R)-6-chloro-3-((1-(2- (isoindolin-2-yl)-3,7-dimethyl-4- oxo-4H-pyrido[1,2-a]pyrimidin-9- yl)ethyl)amino)picolinic acid |
| 10-31 | 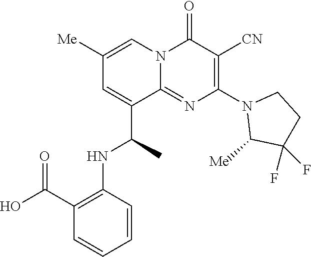 [SMILES: Cc1cc([C@@H](C)Nc2ccccc2C(=O)O)c2nc(N3CCC(F)(F)[C@@H]3C)c(C#N)c(=O)n2c1] | 2-(((R)-1-(3-cyano-2-((S)- 3,3-difluoro-2-methylpyrrolidin-1- yl)-7-methyl-4-oxo-4H-pyrido[1,2- a]pyrimidin-9- yl)ethyl)amino)benzoic acid |
| 10-32 | 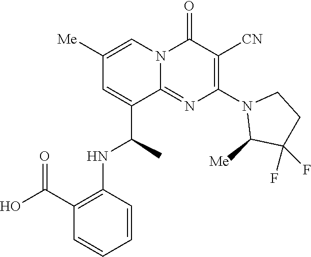 [SMILES: Cc1cc([C@@H](C)Nc2ccccc2C(=O)O)c2nc(N3CCC(F)(F)[C@H]3C)c(C#N)c(=O)n2c1] | 2-(((R)-1-(3-cyano-2-((R)- 3,3-difluoro-2-methylpyrrolidin-1- yl)-7-methyl-4-oxo-4H-pyrido[1,2- a]pyrimidin-9- yl)ethyl)amino)benzoic acid |
| 10-33 | 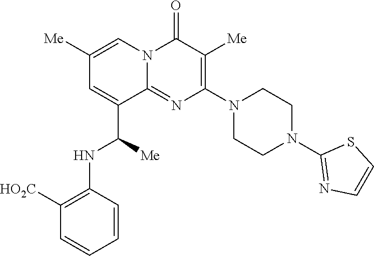 [SMILES: Cc1cc([C@@H](C)Nc2ccccc2C(=O)O)c2nc(N3CCN(c4nccs4)CC3)c(C)c(=O)n2c1] | (R)-2-((1-(3,7-dimethyl-4- oxo-2-(4-(thiazol-2-yl)piperazin-1- yl)-4H-pyrido[1,2-a]pyrimidin-9- yl)ethyl)amino)benzoic acid |
| 10-34 | 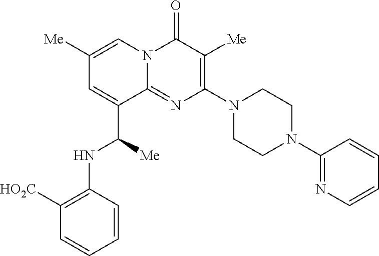 [SMILES: Cc1cc([C@@H](C)Nc2ccccc2C(=O)O)c2nc(N3CCN(c4ccccn4)CC3)c(C)c(=O)n2c1] | (R)-2-((1-(3,7-dimethyl-4- oxo-2-(4-(pyridin-2-yl)piperazin-1- yl)-4H-pyrido[1,2-a]pyrimidin-9- yl)ethyl)amino)benzoic acid |
| 10-35 | 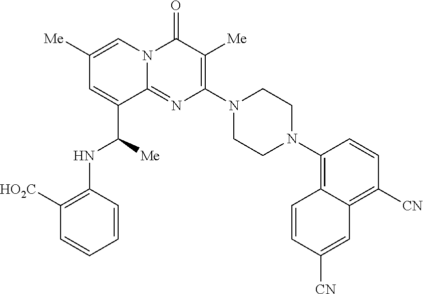 [SMILES: Cc1cc([C@@H](C)Nc2ccccc2C(=O)O)c2nc(N3CCN(c4ccc(C#N)c5cc(C#N)ccc45)CC3)c(C)c(=O)n2c1] | (R)-2-((1-(2-(4-(4,6- dicyanonaphthalen-1-yl)piperazin- 1-yl)-3,7-dimethyl-4-oxo-4H- pyrido[1,2-a]pyrimidin-9- yl)ethyl)amino)benzoic acid |
| 10-36 | 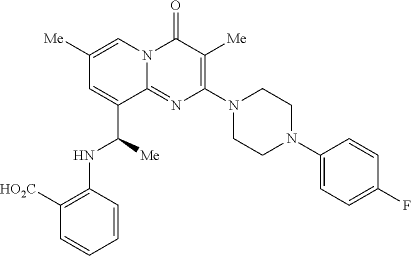 [SMILES: Cc1cc([C@@H](C)Nc2ccccc2C(=O)O)c2nc(N3CCN(c4ccc(F)cc4)CC3)c(C)c(=O)n2c1] | (R)-2-((1-(2-(4-(4- fluorophenyl)piperazin-1-yl)-3,7- dimethyl-4-oxo-4H-pyrido[1,2- a]pyrimidin-9- yl)ethyl)amino)benzoic acid |
| 10-37 | 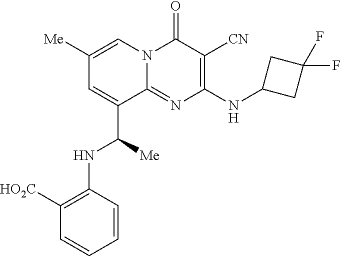 [SMILES: Cc1cc([C@@H](C)Nc2ccccc2C(=O)O)c2nc(NC3CC(F)(F)C3)c(C#N)c(=O)n2c1] | (R)-2-((1-(3-cyano-2-((3,3- difluorocyclobutyl)amino)-7- methyl-4-oxo-4H-pyrido[1,2- a]pyrimidin-9- yl)ethyl)amino)benzoic acid |
| 10-38 | 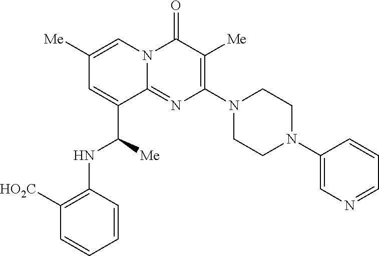 [SMILES: Cc1cc([C@@H](C)Nc2ccccc2C(=O)O)c2nc(N3CCN(c4cccnc4)CC3)c(C)c(=O)n2c1] | (R)-2-((1-(3,7-dimethyl-4- oxo-2-(4-(pyridin-3-yl)piperazin-1- yl)-4H-pyrido[1,2-a]pyrimidin-9- yl)ethyl)amino)benzoic acid |
| 10-39 | 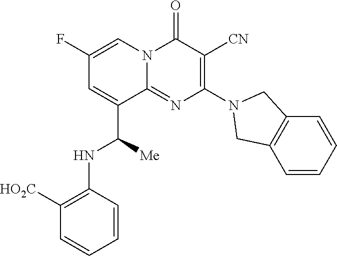 [SMILES: C[C@@H](Nc1ccccc1C(=O)O)c1cc(F)cn2c(=O)c(C#N)c(N3Cc4ccccc4C3)nc12] | (R)-2-((1-(3-cyano-7-fluoro- 2-(isoindolin-2-yl)-4-oxo-4H- pyrido[1,2-a]pyrimidin-9- yl)ethyl)amino)benzoic acid |
| 10-40 | 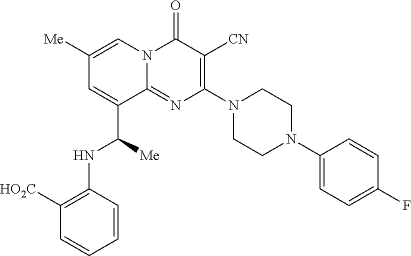 [SMILES: Cc1cc([C@@H](C)Nc2ccccc2C(=O)O)c2nc(N3CCN(c4ccc(F)cc4)CC3)c(C#N)c(=O)n2c1] | (R)-2-((1-(3-cyano-2-(4-(4- fluorophenyl)piperazin-1-yl)-7- methyl-4-oxo-4H-pyrido[1,2- a]pyrimidin-9- yl)ethyl)amino)benzoic acid |
| 10-41 | 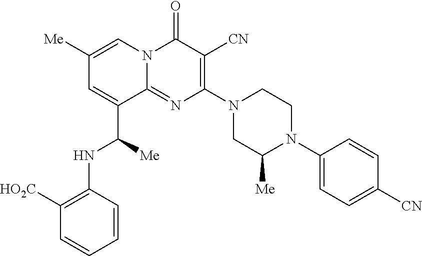 [SMILES: Cc1cc([C@@H](C)Nc2ccccc2C(=O)O)c2nc(N3CCN(c4ccc(C#N)cc4)[C@@H](C)C3)c(C#N)c(=O)n2c1] | 2-(((R)-1-(3-cyano-2-((S)-4- (4-cyanophenyl)-3- methylpiperazin-1-yl)-7-methyl-4- oxo-4H-pyrido[1,2-a]pyrimidin-9- yl)ethyl)amino)benzoic acid |
| 10-42 | 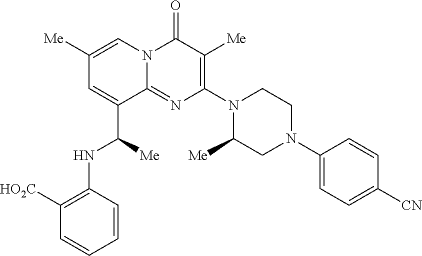 [SMILES: Cc1cc([C@@H](C)Nc2ccccc2C(=O)O)c2nc(N3CCN(c4ccc(C#N)cc4)C[C@H]3C)c(C)c(=O)n2c1] | 2-(((R)-1-(2-((R)-4-(4- cyanophenyl)-2-methylpiperazin- 1-yl)-3,7-dimethyl-4-oxo-4H- pyrido[1,2-a]pyrimidin-9- yl)ethyl)amino)benzoic acid |
| 10-43 | 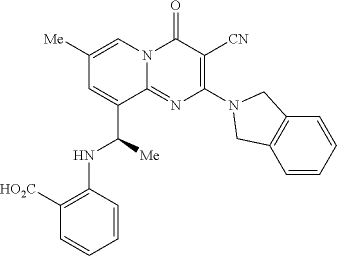 [SMILES: Cc1cc([C@@H](C)Nc2ccccc2C(=O)O)c2nc(N3Cc4ccccc4C3)c(C#N)c(=O)n2c1] | (R)-2-((1-(3-cyano-2- (isoindolin-2-yl)-7-methyl-4-oxo- 4H-pyrido[1,2-a]pyrimidin-9- yl)ethyl)amino)benzoic acid |
| 10-44 | 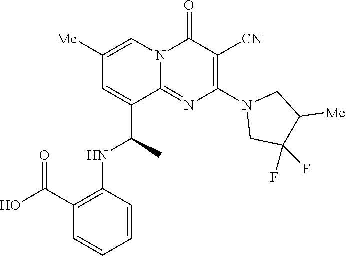 [SMILES: Cc1cc([C@@H](C)Nc2ccccc2C(=O)O)c2nc(N3CC(C)C(F)(F)C3)c(C#N)c(=O)n2c1] | 2-(((1R)-1-(3-cyano-2-(3,3- difluoro-4-methylpyrrolidin-1-yl)-7- methyl-4-oxo-4H-pyrido[1,2- a]pyrimidin-9- yl)ethyl)amino)benzoic acid |
| 10-45 | 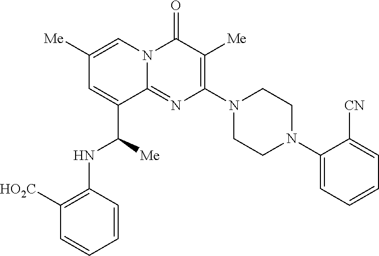 [SMILES: Cc1cc([C@@H](C)Nc2ccccc2C(=O)O)c2nc(N3CCN(c4ccccc4C#N)CC3)c(C)c(=O)n2c1] | (R)-2-((1-(2-(4-(2- cyanophenyl)piperazin-1-yl)-3,7- dimethyl-4-oxo-4H-pyrido[1,2- a]pyrimidin-9- yl)ethyl)amino)benzoic acid |
| 10-46 | 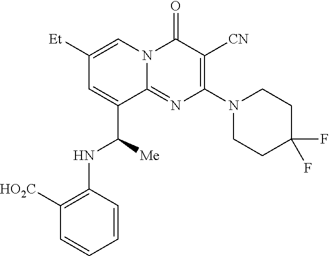 [SMILES: CCc1cc([C@@H](C)Nc2ccccc2C(=O)O)c2nc(N3CCC(F)(F)CC3)c(C#N)c(=O)n2c1] | (R)-2-((1-(3-cyano-2-(4,4- difluoropiperidin-1-yl)-7-ethyl-4- oxo-4H-pyrido[1,2-a]pyrimidin-9- yl)ethyl)amino)benzoic acid |
| 10-47 | 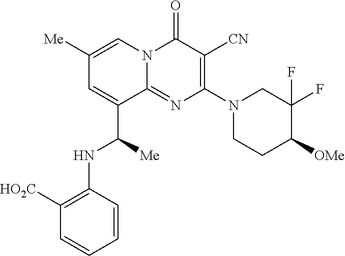 [SMILES: CO[C@H]1CCN(c2nc3c([C@@H](C)Nc4ccccc4C(=O)O)cc(C)cn3c(=O)c2C#N)CC1(F)F] | 2-(((R)-1-(3-cyano-2-((S)- 3,3-difluoro-4-methoxypiperidin-1- yl)-7-methyl-4-oxo-4H-pyrido[1,2- a]pyrimidin-9- yl)ethyl)amino)benzoic acid |
| | 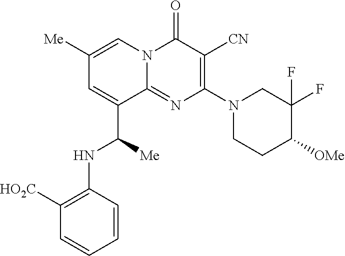 [SMILES: CO[C@@H]1CCN(c2nc3c([C@@H](C)Nc4ccccc4C(=O)O)cc(C)cn3c(=O)c2C#N)CC1(F)F] | 2-(((R)-1-(3-cyano-2-((R)- 3,3-difluoro-4-methoxypiperidin-1- yl)-7-methyl-4-oxo-4H-pyrido[1,2- a]pyrimidin-9- yl)ethyl)amino)benzoic acid |
| 10-48 | 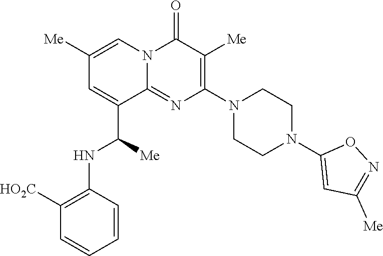 [SMILES: Cc1cc([C@@H](C)Nc2ccccc2C(=O)O)c2nc(N3CCN(c4cc(C)no4)CC3)c(C)c(=O)n2c1] | (R)-2-((1-(3,7-dimethyl-2-(4- (3-methylisoxazol-5-yl)piperazin- 1-yl)-4-oxo-4H-pyrido[1,2- a]pyrimidin-9- yl)ethyl)amino)benzoic acid |
| 10-49 | 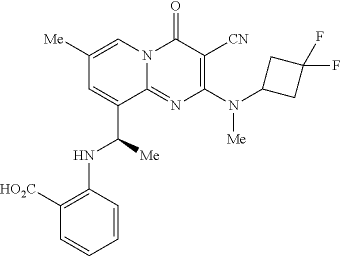 [SMILES: Cc1cc([C@@H](C)Nc2ccccc2C(=O)O)c2nc(N(C)C3CC(F)(F)C3)c(C#N)c(=O)n2c1] | (R)-2-((1-(3-cyano-2-((3,3- difluorocyclobutyl)(methyl)amino)- 7-methyl-4-oxo-4H-pyrido[1,2- a]pyrimidin-9- yl)ethyl)amino)benzoic acid |
| 10-50 | 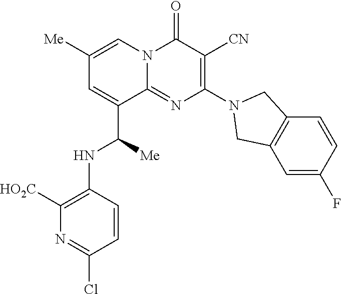 [SMILES: Cc1cc([C@@H](C)Nc2ccc(Cl)nc2C(=O)O)c2nc(N3Cc4ccc(F)cc4C3)c(C#N)c(=O)n2c1] | (R)-6-chloro-3-((1-(3-cyano- 2-(5-fluoroisoindolin-2-yl)-7- methyl-4-oxo-4H-pyrido[1,2- a]pyrimidin-9- yl)ethyl)amino)picolinic acid |
| 10-51 | 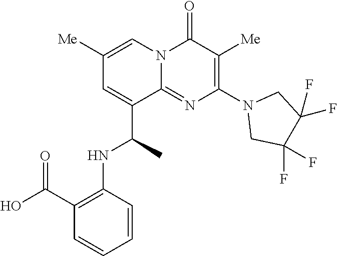 [SMILES: Cc1cc([C@@H](C)Nc2ccccc2C(=O)O)c2nc(N3CC(F)(F)C(F)(F)C3)c(C)c(=O)n2c1] | (R)-2-((1-(3,7-dimethyl-4- oxo-2-(3,3,4,4- tetrafluoropyrrolidin-1-yl)-4H- pyrido[1,2-a]pyrimidin-9- yl)ethyl)amino)benzoic acid |
| 10-52 | 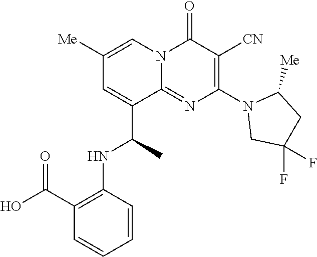 [SMILES: Cc1cc([C@@H](C)Nc2ccccc2C(=O)O)c2nc(N3CC(F)(F)C[C@H]3C)c(C#N)c(=O)n2c1] | 2-(((R)-1-(3-cyano-2-((R)- 4,4-difluoro-2-methylpyrrolidin-1- yl)-7-methyl-4-oxo-4H-pyrido[1,2- a]pyrimidin-9- yl)ethyl)amino)benzoic acid |
| 10--52 | 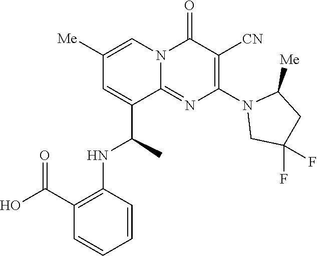 [SMILES: Cc1cc([C@@H](C)Nc2ccccc2C(=O)O)c2nc(N3CC(F)(F)C[C@@H]3C)c(C#N)c(=O)n2c1] | 2-(((R)-1-(3-cyano-2-((S)- 4,4-difluoro-2-methylpyrrolidin-1- yl)-7-methyl-4-oxo-4H-pyrido[1,2- a]pyrimidin-9- yl)ethyl)amino)benzoic acid |
| 10-53 | 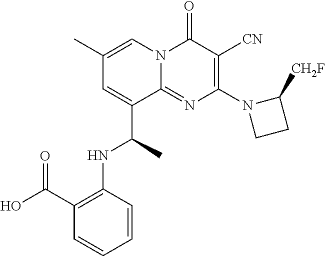 | 2-(((R)-1-(3-cyano-2-((R)-2- (fluoromethyl)azetidin-1-yl)-7- methyl-4-oxo-4H-pyrido[1,2- a]pyrimidin-9- yl)ethyl)amino)benzoic acid |
| 10-54 | 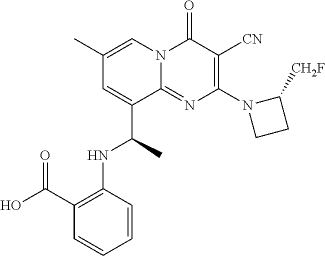 | 2-(((R)-1-(3-cyano-2-((S)-2- (fluoromethyl)azetidin-1-yl)-7- methyl-4-oxo-4H-pyrido[1,2- a]pyrimidin-9- yl)ethyl)amino)benzoic acid |
| 10-55 | 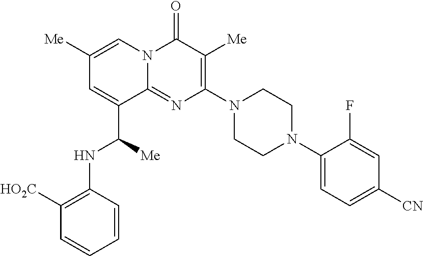 [SMILES: Cc1cc([C@@H](C)Nc2ccccc2C(=O)O)c2nc(N3CCN(c4ccc(C#N)cc4F)CC3)c(C)c(=O)n2c1] | (R)-2-((1-(2-(4-(4-cyano-2- fluorophenyl)piperazin-1-yl)-3,7- dimethyl-4-oxo-4H-pyrido[1,2- a]pyrimidin-9- yl)ethyl)amino)benzoic acid |
| 10-56 | 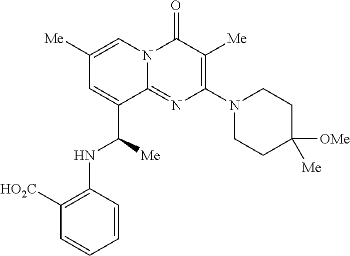 [SMILES: COC1(C)CCN(c2nc3c([C@@H](C)Nc4ccccc4C(=O)O)cc(C)cn3c(=O)c2C)CC1] | (R)-2-((1-(2-(4-methoxy-4- methylpiperidin-1-yl)-3,7-dimethyl- 4-oxo-4H-pyrido[1,2-a]pyrimidin- 9-yl)ethyl)amino)benzoic acid |
| 10-57 | 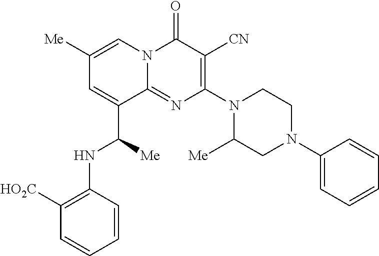 [SMILES: Cc1cc([C@@H](C)Nc2ccccc2C(=O)O)c2nc(N3CCN(c4ccccc4)CC3C)c(C#N)c(=O)n2c1] | 2-(((1R)-1-(3-cyano-7- methyl-2-(2-methyl-4- phenylpiperazin-1-yl)-4-oxo-4H- pyrido[1,2-a]pyrimidin-9- yl)ethyl)amino)benzoic acid |
| 10-58 | 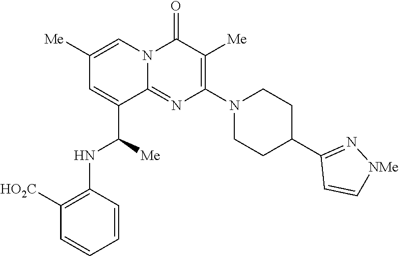 [SMILES: Cc1cc([C@@H](C)Nc2ccccc2C(=O)O)c2nc(N3CCC(c4ccn(C)n4)CC3)c(C)c(=O)n2c1] | (R)-2-((1-(3,7-dimethyl-2-(4- (1-methyl-1H-pyrazol-3- yl)piperidin-1-yl)-4-oxo-4H- pyrido[1,2-a]pyrimidin-9- yl)ethyl)amino)benzoic acid |
| 10-59 | 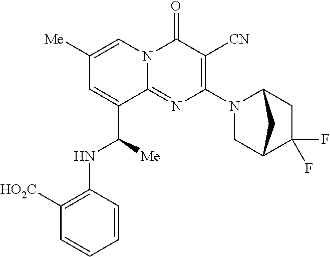 [SMILES: Cc1cc([C@@H](C)Nc2ccccc2C(=O)O)c2nc(N3C[C@@H]4C[C@H]3CC4(F)F)c(C#N)c(=O)n2c1] | 2-(((R)-1-(3-cyano-2- ((1S,4S)-5,5-difluoro-2- azabicyclo[2.2.1]heptan-2-yl)-7- methyl-4-oxo-4H-pyrido[1,2- a]pyrimidin-9- yl)ethyl)amino)benzoic acid |
| 10-59 | 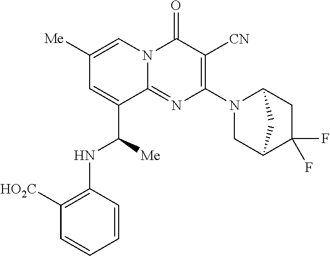 [SMILES: Cc1cc([C@@H](C)Nc2ccccc2C(=O)O)c2nc(N3C[C@H]4C[C@@H]3CC4(F)F)c(C#N)c(=O)n2c1] | 2-(((R)-1-(3-cyano-2- ((1R,4R)-5,5-difluoro-2- azabicyclo[2.2.1]heptan-2-yl)-7- methyl-4-oxo-4H-pyrido[1,2- a]pyrimidin-9- yl)ethyl)amino)benzoic acid |
| 10-60 | 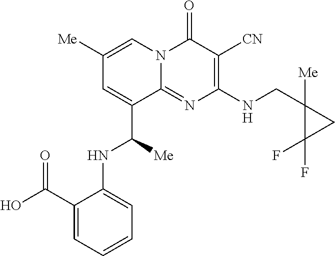 [SMILES: Cc1cc([C@@H](C)Nc2ccccc2C(=O)O)c2nc(NCC3(C)CC3(F)F)c(C#N)c(=O)n2c1] | 2-(((1R)-1-(3-cyano-2- (((2,2-difluoro-1- methylcyclopropyl)methyl)amino)- 7-methyl-4-oxo-4H-pyrido[1,2- a]pyrimidin-9- yl)ethyl)amino)benzoic acid |
| 10-61 | 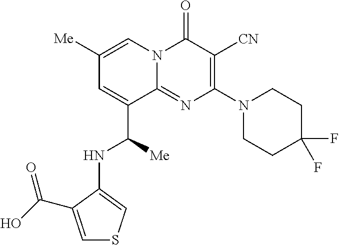 [SMILES: Cc1cc([C@@H](C)Nc2cscc2C(=O)O)c2nc(N3CCC(F)(F)CC3)c(C#N)c(=O)n2c1] | (R)-4-((1-(3-cyano-2-(4,4- difluoropiperidin-1-yl)-7-methyl-4- oxo-4H-pyrido[1,2-a]pyrimidin-9- yl)ethyl)amino)thiophene-3- carboxylic acid |
| 10-62 | 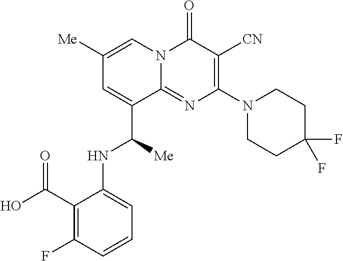 [SMILES: Cc1cc([C@@H](C)Nc2cccc(F)c2C(=O)O)c2nc(N3CCC(F)(F)CC3)c(C#N)c(=O)n2c1] | (R)-2-((1-(3-cyano-2-(4,4- difluoropiperidin-1-yl)-7-methyl-4- oxo-4H-pyrido[1,2-a]pyrimidin-9- yl)ethyl)amino)-6-fluorobenzoic acid |
| 10-63 | 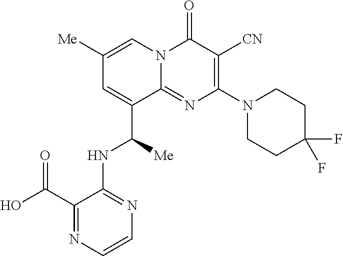 [SMILES: Cc1cc([C@@H](C)Nc2nccnc2C(=O)O)c2nc(N3CCC(F)(F)CC3)c(C#N)c(=O)n2c1] | (R)-3-((1-(3-cyano-2-(4,4- difluoropiperidin-1-yl)-7-methyl-4- oxo-4H-pyrido[1,2-a]pyrimidin-9- yl)ethyl)amino)pyrazine-2- carboxylic acid |
| 10-64 | 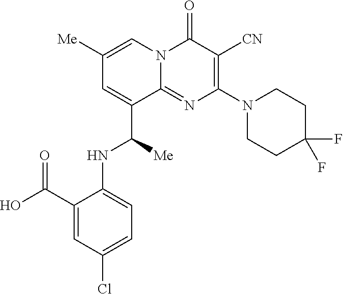 [SMILES: Cc1cc([C@@H](C)Nc2ccc(Cl)cc2C(=O)O)c2nc(N3CCC(F)(F)CC3)c(C#N)c(=O)n2c1] | (R)-5-chloro-2-((1-(3-cyano- 2-(4,4-difluoropiperidin-1-yl)-7- methyl-4-oxo-4H-pyrido[1,2- a]pyrimidin-9- yl)ethyl)amino)benzoic acid |
| 10-65 | 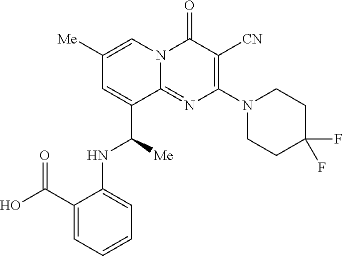 [SMILES: Cc1cc([C@@H](C)Nc2ccccc2C(=O)O)c2nc(N3CCC(F)(F)CC3)c(C#N)c(=O)n2c1] | (R)-2-((1-(3-cyano-2-(4,4- difluorocyclohexyl)-7-methyl-4- oxo-4H-pyrido[1,2-a]pyrimidin-9- yl)ethyl)amino)benzoic acid |
| 10-66 | 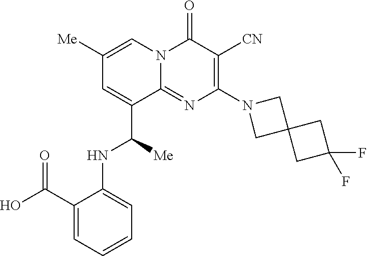 [SMILES: Cc1cc([C@@H](C)Nc2ccccc2C(=O)O)c2nc(N3CC4(C3)CC(F)(F)C4)c(C#N)c(=O)n2c1] | (R)-2-((1-(3-cyano-2-(6,6- difluoro-2-azaspiro[3.3]heptan-2- yl)-7-methyl-4-oxo-4H-pyrido[1,2- a]pyrimidin-9- yl)ethyl)amino)benzoic acid |
| 10-67 | 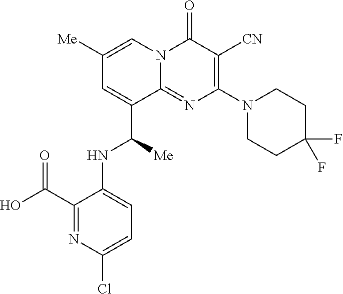 [SMILES: Cc1cc([C@@H](C)Nc2ccc(Cl)nc2C(=O)O)c2nc(N3CCC(F)(F)CC3)c(C#N)c(=O)n2c1] | (R)-6-chloro-3-((1-(3-cyano- 2-(4,4-difluoropiperidin-1-yl)-7- methyl-4-oxo-4H-pyrido[1,2- a]pyrimidin-9- yl)ethyl)amino)picolinic acid |
| 10-68 | 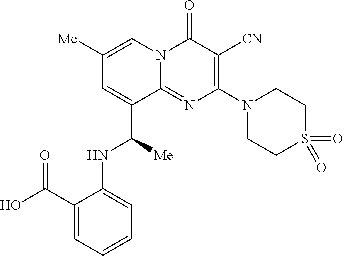 [SMILES: Cc1cc([C@@H](C)Nc2ccccc2C(=O)O)c2nc(N3CCS(=O)(=O)CC3)c(C#N)c(=O)n2c1] | (R)-2-((1-(3-cyano-2-(1,1- dioxidothiomorpholino)-7-methyl- 4-oxo-4H-pyrido[1,2-a]pyrimidin- 9-yl)ethyl)amino)benzoic acid |
| 10-69 | 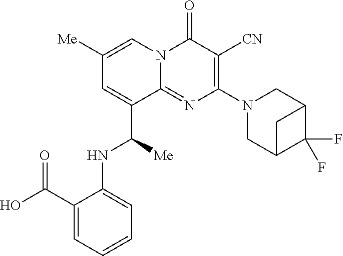 [SMILES: Cc1cc([C@@H](C)Nc2ccccc2C(=O)O)c2nc(N3CC4CC(C3)C4(F)F)c(C#N)c(=O)n2c1] | 2-(((1R)-1-(3-cyano-2-(6,6- difluoro-3- azabicyclo[3.1.1]heptan-3-yl)-7- methyl-4-oxo-4H-pyrido[1,2- a]pyrimidin-9- yl)ethyl)amino)benzoic acid |
| 10-70 | 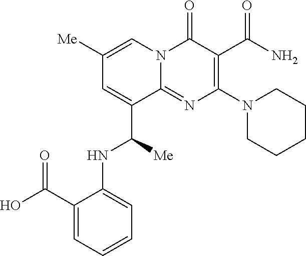 [SMILES: Cc1cc([C@@H](C)Nc2ccccc2C(=O)O)c2nc(N3CCCCC3)c(C(N)=O)c(=O)n2c1] | (R)-2-((1-(3-carbamoyl-7- methyl-4-oxo-2-(piperidin-1-yl)- 4H-pyrido[1,2-a]pyrimidin-9- yl)ethyl)amino)benzoic acid |
| 10-71 | 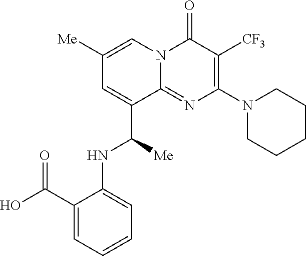 [SMILES: Cc1cc([C@@H](C)Nc2ccccc2C(=O)O)c2nc(N3CCCCC3)c(C(F)(F)F)c(=O)n2c1] | (R)-2-((1-(7-methyl-4-oxo-2- (piperidin-1-yl)-3-(trifluoromethyl)- 4H-pyrido[1,2-a]pyrimidin-9- yl)ethyl)amino)benzoic acid |
| 10-72 | 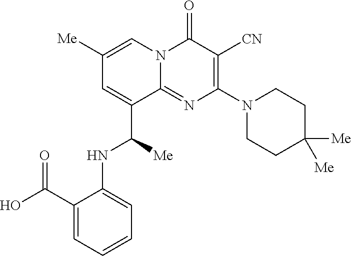 [SMILES: Cc1cc([C@@H](C)Nc2ccccc2C(=O)O)c2nc(N3CCC(C)(C)CC3)c(C#N)c(=O)n2c1] | (R)-2-((1-(3-cyano-2-(4,4- dimethylpiperidin-1-yl)-7-methyl-4- oxo-4H-pyrido[1,2-a]pyrimidin-9- yl)ethyl)amino)benzoic acid |
| 10-73 | 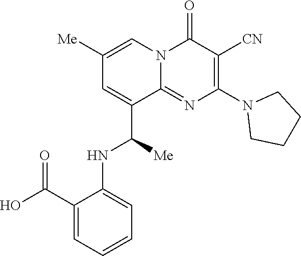 [SMILES: Cc1cc([C@@H](C)Nc2ccccc2C(=O)O)c2nc(N3CCCC3)c(C#N)c(=O)n2c1] | (R)-2-((1-(3-cyano-7- methyl-4-oxo-2-(pyrrolidin-1-yl)- 4H-pyrido[1,2-a]pyrimidin-9- yl)ethyl)amino)benzoic acid |
| 10-74 | 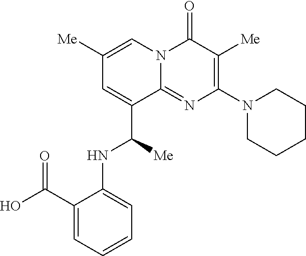 [SMILES: Cc1cc([C@@H](C)Nc2ccccc2C(=O)O)c2nc(N3CCCCC3)c(C)c(=O)n2c1] | (R)-2-((1-(3,7-dimethyl-4- oxo-2-(piperidin-1-yl)-4H- pyrido[1,2-a]pyrimidin-9- yl)ethyl)amino)benzoic acid |
| 10-75 | 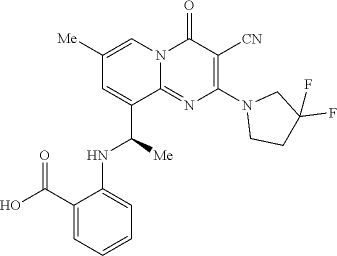 [SMILES: Cc1cc([C@@H](C)Nc2ccccc2C(=O)O)c2nc(N3CCC(F)(F)C3)c(C#N)c(=O)n2c1] | (R)-2-((1-(3-cyano-2-(3,3- difluoropyrrolidin-1-yl)-7-methyl-4- oxo-4H-pyrido[1,2-a]pyrimidin-9- yl)ethyl)amino)benzoic acid |
| 10-76 | 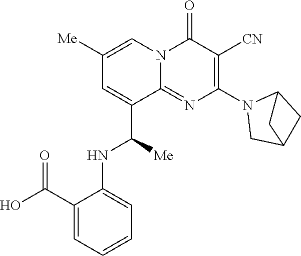 [SMILES: Cc1cc([C@@H](C)Nc2ccccc2C(=O)O)c2nc(N3CC4CC3C4)c(C#N)c(=O)n2c1] | (R)-2-((1-(2-(2- azabicyclo[2.1.1]hexan-2-yl)-3- cyano-7-methyl-4-oxo-4H- pyrido[1,2-a]pyrimidin-9- yl)ethyl)amino)benzoic acid |
| 10-77 | 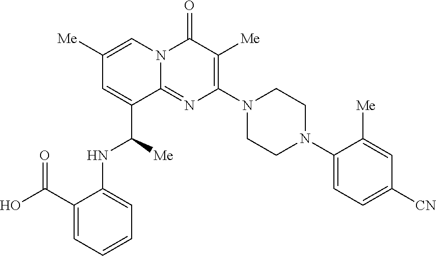 [SMILES: Cc1cc([C@@H](C)Nc2ccccc2C(=O)O)c2nc(N3CCN(c4ccc(C#N)cc4C)CC3)c(C)c(=O)n2c1] | (R)-2-((1-(2-(4-(4-cyano-2- methylphenyl)piperazin-1-yl)-3,7- dimethyl-4-oxo-4H-pyrido[1,2- a]pyrimidin-9- yl)ethyl)amino)benzoic acid |
| 10-78 | 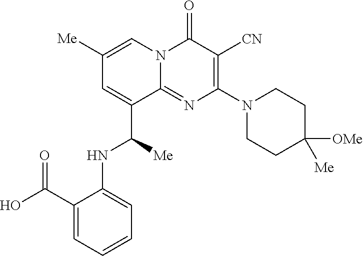 [SMILES: COC1(C)CCN(c2nc3c([C@@H](C)Nc4ccccc4C(=O)O)cc(C)cn3c(=O)c2C#N)CC1] | (R)-2-((1-(3-cyano-2-(4- methoxy-4-methylpiperidin-1-yl)- 7-methyl-4-oxo-4H-pyrido[1,2- a]pyrimidin-9- yl)ethyl)amino)benzoic acid |
| 10-79 | 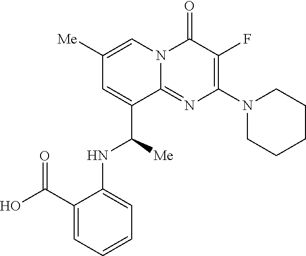 [SMILES: Cc1cc([C@@H](C)Nc2ccccc2C(=O)O)c2nc(N3CCCCC3)c(F)c(=O)n2c1] | (R)-2-((1-(3-fluoro-7-methyl- 4-oxo-2-(piperidin-1-yl)-4H- pyrido[1,2-a]pyrimidin-9- yl)ethyl)amino)benzoic acid |
| 10-80 | 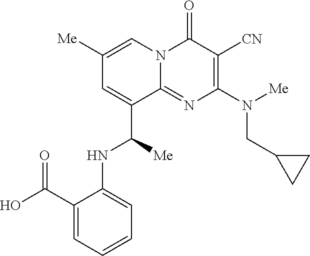 [SMILES: Cc1cc([C@@H](C)Nc2ccccc2C(=O)O)c2nc(N(C)CC3CC3)c(C#N)c(=O)n2c1] | (R)-2-((1-(3-cyano-2- ((cyclopropylmethyl)(methyl)amin0)- 7-methyl-4-oxo-4H-pyrido[1,2- a]pyrimidin-9- yl)ethyl)amino)benzoic acid |
| 10-81 | 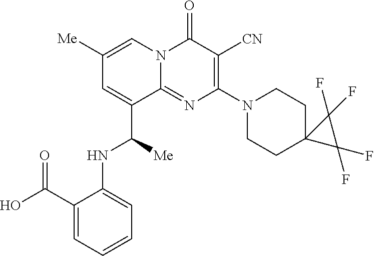 [SMILES: Cc1cc([C@@H](C)Nc2ccccc2C(=O)O)c2nc(N3CCC4(CC3)C(F)(F)C4(F)F)c(C#N)c(=O)n2c1] | (R)-2-((1-(3-cyano-7- methyl-4-oxo-2-(1,1,2,2- tetrafluoro-6-azaspiro[2.5]octan-6- yl)-4H-pyrido[1,2-a]pyrimidin-9- yl)ethyl)amino)benzoic acid |
| 10-82 | 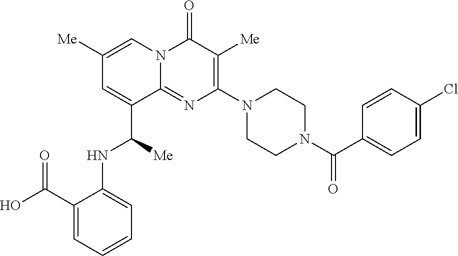 [SMILES: Cc1cc([C@@H](C)Nc2ccccc2C(=O)O)c2nc(N3CCN(C(=O)c4ccc(Cl)cc4)CC3)c(C)c(=O)n2c1] | (R)-2-((1-(2-(4-(4- chlorobenzoyl)piperazin-1-yl)-3,7- dimethyl-4-oxo-4H-pyrido[1,2- a]pyrimidin-9- yl)ethyl)amino)benzoic acid |
| 10-83 | 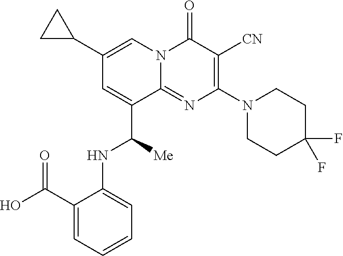 [SMILES: C[C@@H](Nc1ccccc1C(=O)O)c1cc(C2CC2)cn2c(=O)c(C#N)c(N3CCC(F)(F)CC3)nc12] | (R)-2-((1-(3-cyano-7- cyclopropyl-2-(4,4- difluoropiperidin-1-yl)-4-oxo-4H- pyrido[1,2-a]pyrimidin-9- yl)ethyl)amino)benzoic acid |
| 10-84 | 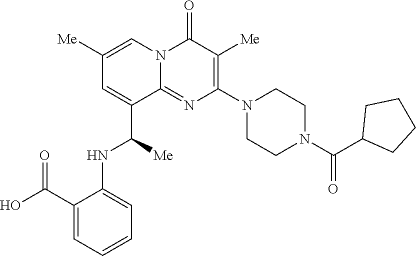 [SMILES: Cc1cc([C@@H](C)Nc2ccccc2C(=O)O)c2nc(N3CCN(C(=O)C4CCCC4)CC3)c(C)c(=O)n2c1] | (R)-2-((1-(2-(4- (cyclopentanecarbonyl)piperazin- 1-yl)-3,7-dimethyl-4-oxo-4H- pyrido[1,2-a]pyrimidin-9- yl)ethyl)amino)benzoic acid |
| 10-85 | 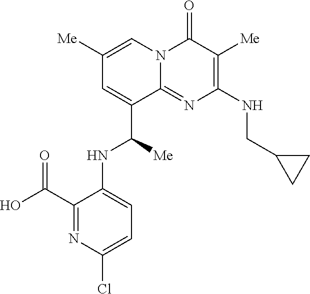 [SMILES: Cc1cc([C@@H](C)Nc2ccc(Cl)nc2C(=O)O)c2nc(NCC3CC3)c(C)c(=O)n2c1] | (R)-6-chloro-3-((1-(2- ((cyclopropylmethyl)amino)-3,7- dimethyl-4-oxo-4H-pyrido[1,2- a]pyrimidin-9- yl)ethyl)amino)picolinic acid |
| 10-86 | 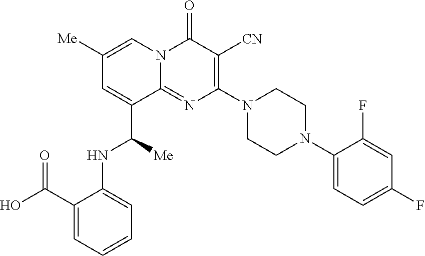 [SMILES: Cc1cc([C@@H](C)Nc2ccccc2C(=O)O)c2nc(N3CCN(c4ccc(F)cc4F)CC3)c(C#N)c(=O)n2c1] | (R)-2-((1-(3-cyano-2-(4- (2,4-difluorophenyl)piperazin-1- yl)-7-methyl-4-oxo-4H-pyrido[1,2- a]pyrimidin-9- yl)ethyl)amino)benzoic acid |
| 10-87 | 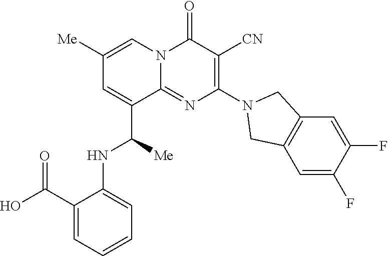 [SMILES: Cc1cc([C@@H](C)Nc2ccccc2C(=O)O)c2nc(N3Cc4cc(F)c(F)cc4C3)c(C#N)c(=O)n2c1] | (R)-2-((1-(3-cyano-2-(5,6- difluoroisoindolin-2-yl)-7-methyl-4- oxo-4H-pyrido[1,2-a]pyrimidin-9- yl)ethyl)amino)benzoic acid |
| 10-88 | 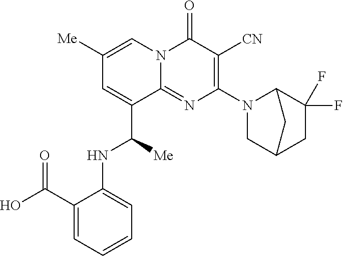 [SMILES: Cc1cc([C@@H](C)Nc2ccccc2C(=O)O)c2nc(N3CC4CC3C(F)(F)C4)c(C#N)c(=O)n2c1] | 2-(((1R)-1-(3-cyano-2-(6,6- difluoro-2- azabicyclo[2.2.1]heptan-2-yl)-7- methyl-4-oxo-4H-pyrido[1,2- a]pyrimidin-9- yl)ethyl)amino)benzoic acid |
| 10-89 | 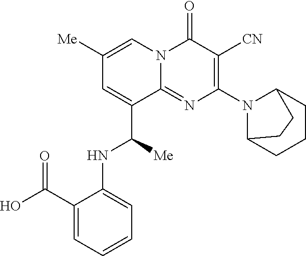 [SMILES: Cc1cc([C@@H](C)Nc2ccccc2C(=O)O)c2nc(N3C4CCCC3CC4)c(C#N)c(=O)n2c1] | 2-(((1R)-1-(2-(8- azabicyclo[3.2.1]octan-8-yl)-3- cyano-7-methyl-4-oxo-4H- pyrido[1,2-a]pyrimidin-9- yl)ethyl)amino)benzoic acid |
| 10-90 | 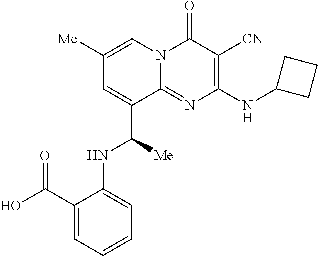 [SMILES: Cc1cc([C@@H](C)Nc2ccccc2C(=O)O)c2nc(NC3CCC3)c(C#N)c(=O)n2c1] | (R)-2-((1-(3-cyano-2- (cyclobutylamino)-7-methyl-4-oxo- 4H-pyrido[1,2-a]pyrimidin-9- yl)ethyl)amino)benzoic acid |
| 10-91 | 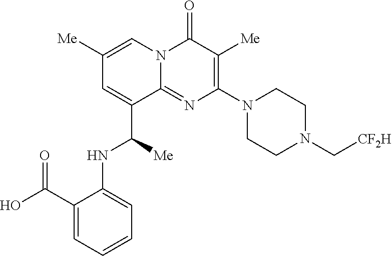 [SMILES: Cc1cc([C@@H](C)Nc2ccccc2C(=O)O)c2nc(N3CCN(CC(F)F)CC3)c(C)c(=O)n2c1] | (R)-2-((1-(2-(4-(2,2- difluoroethyl)piperazin-1-yl)-3,7- dimethyl-4-oxo-4H-pyrido[1,2- a]pyrimidin-9- yl)ethyl)amino)benzoic acid |
| 10-92 | 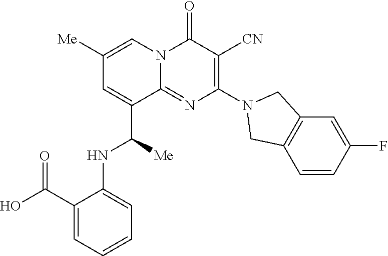 [SMILES: Cc1cc([C@@H](C)Nc2ccccc2C(=O)O)c2nc(N3Cc4ccc(F)cc4C3)c(C#N)c(=O)n2c1] | (R)-2-((1-(3-cyano-2-(5- fluoroisoindolin-2-yl)-7-methyl-4- oxo-4H-pyrido[1,2-a]pyrimidin-9- yl)ethyl)amino)benzoic acid |
| 10-93 | 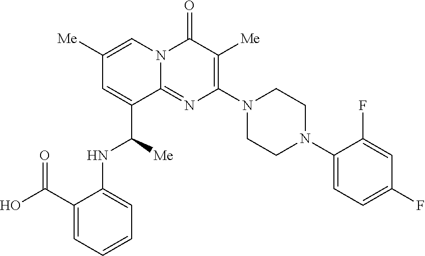 [SMILES: Cc1cc([C@@H](C)Nc2ccccc2C(=O)O)c2nc(N3CCN(c4ccc(F)cc4F)CC3)c(C)c(=O)n2c1] | (R)-2-((1-(2-(4-(2,4- difluorophenyl)piperazin-1-yl)-3,7- dimethyl-4-oxo-4H-pyrido[1,2- a]pyrimidin-9- yl)ethyl)amino)benzoic acid |
| 10-94 | 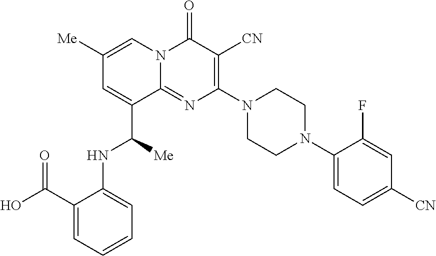 [SMILES: Cc1cc([C@@H](C)Nc2ccccc2C(=O)O)c2nc(N3CCN(c4ccc(C#N)cc4F)CC3)c(C#N)c(=O)n2c1] | (R)-2-((1-(3-cyano-2-(4-(4- cyano-2-fluorophenyl)piperazin-1- yl)-7-methyl-4-oxo-4H-pyrido[1,2- a]pyrimidin-9- yl)ethyl)amino)benzoic acid |
| 10-95 | 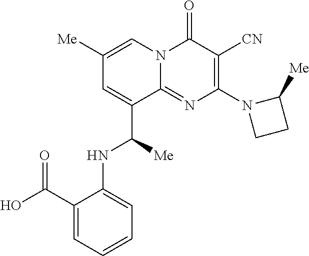 [SMILES: Cc1cc([C@@H](C)Nc2ccccc2C(=O)O)c2nc(N3CC[C@@H]3C)c(C#N)c(=O)n2c1] | 2-(((R)-1-(3-cyano-7- methyl-2-((S)-2-methylazetidin-1- yl)-4-oxo-4H-pyrido[1,2- a]pyrimidin-9- yl)ethyl)amino)benzoic acid |
| 10-96 | 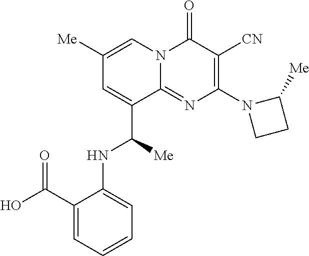 [SMILES: Cc1cc([C@@H](C)Nc2ccccc2C(=O)O)c2nc(N3CC[C@H]3C)c(C#N)c(=O)n2c1] | 2-(((R)-1-(3-cyano-7- methyl-2-((R)-2-methylazetidin-1- yl)-4-oxo-4H-pyrido[1,2- a]pyrimidin-9- yl)ethyl)amino)benzoic acid |
| 10-97 | 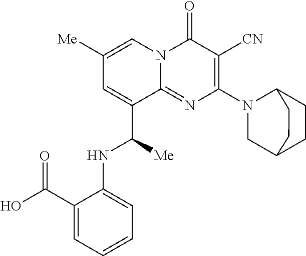 [SMILES: Cc1cc([C@@H](C)Nc2ccccc2C(=O)O)c2nc(N3CC4CCC3CC4)c(C#N)c(=O)n2c1] | (R)-2-((1-(2-(2- azabicyclo[2.2.2]octan-2-yl)-3- cyano-7-methyl-4-oxo-4H- pyrido[1,2-a]pyrimidin-9- yl)ethyl)amino)benzoic acid |
| 10-98 | 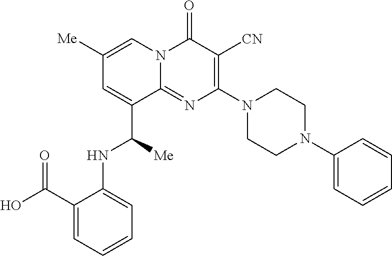 [SMILES: Cc1cc([C@@H](C)Nc2ccccc2C(=O)O)c2nc(N3CCN(c4ccccc4)CC3)c(C#N)c(=O)n2c1] | (R)-2-((1-(3-cyano-7- methyl-4-oxo-2-(4- phenylpiperazin-1-yl)-4H- pyrido[1,2-a]pyrimidin-9- yl)ethyl)amino)benzoic acid |
| 10-99 | 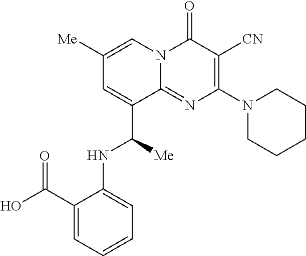 [SMILES: Cc1cc([C@@H](C)Nc2ccccc2C(=O)O)c2nc(N3CCCCC3)c(C#N)c(=O)n2c1] | (R)-2-((1-(3-cyano-7- methyl-4-oxo-2-(piperidin-1-yl)- 4H-pyrido[1,2-a]pyrimidin-9- yl)ethyl)amino)benzoic acid |
| 10-100 | 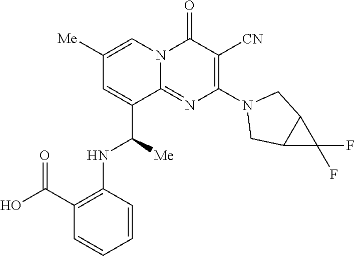 [SMILES: Cc1cc([C@@H](C)Nc2ccccc2C(=O)O)c2nc(N3CC4C(C3)C4(F)F)c(C#N)c(=O)n2c1] | 2-(((1R)-1-(3-cyano-2-(6,6- difluoro-3-azabicyclo[3.1.0]hexan- 3-yl)-7-methyl-4-oxo-4H- pyrido[1,2-a]pyrimidin-9- yl)ethyl)amino)benzoic acid |
| 10-101 | 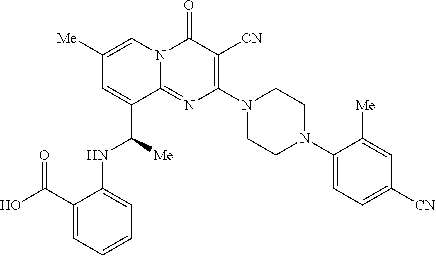 [SMILES: Cc1cc([C@@H](C)Nc2ccccc2C(=O)O)c2nc(N3CCN(c4ccc(C#N)cc4C)CC3)c(C#N)c(=O)n2c1] | (R)-2-((1-(3-cyano-2-(4-(4- cyano-2-methylphenyl)piperazin- 1-yl)-7-methyl-4-oxo-4H- pyrido[1,2-a]pyrimidin-9- yl)ethyl)amino)benzoic acid |
| 10-102 | 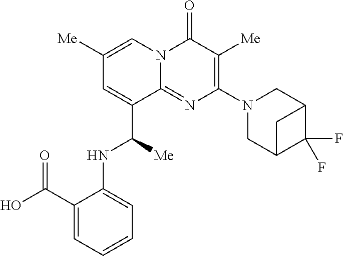 [SMILES: Cc1cc([C@@H](C)Nc2ccccc2C(=O)O)c2nc(N3CC4CC(C3)C4(F)F)c(C)c(=O)n2c1] | 2-(((1R)-1-(2-(6,6-difluoro- 3-azabicyclo[3.1.1]heptan-3-yl)- 3,7-dimethyl-4-oxo-4H-pyrido[1,2- a]pyrimidin-9- yl)ethyl)amino)benzoic acid |
| 10-103 | 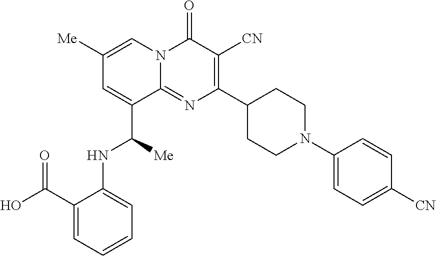 [SMILES: Cc1cc([C@@H](C)Nc2ccccc2C(=O)O)c2nc(C3CCN(c4ccc(C#N)cc4)CC3)c(C#N)c(=O)n2c1] | (R)-2-((1-(3-cyano-2-(1-(4- cyanophenyl)piperidin-4-yl)-7- methyl-4-oxo-4H-pyrido[1,2- a]pyrimidin-9- yl)ethyl)amino)benzoic acid |
| 10-104 | 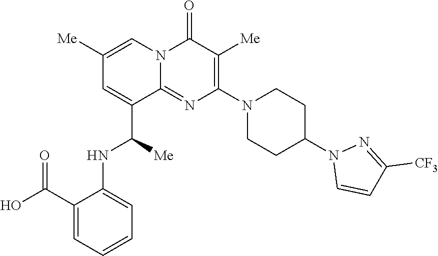 [SMILES: Cc1cc([C@@H](C)Nc2ccccc2C(=O)O)c2nc(N3CCC(n4ccc(C(F)(F)F)n4)CC3)c(C)c(=O)n2c1] | (R)-2-((1-(3,7-dimethyl-4- oxo-2-(4-(3-(trifluoromethyl)-1H- pyrazol-1-yl)piperidin-1-yl)-4H- pyrido[1,2-a]pyrimidin-9- yl)ethyl)amino)benzoic acid |
| 10-105 | 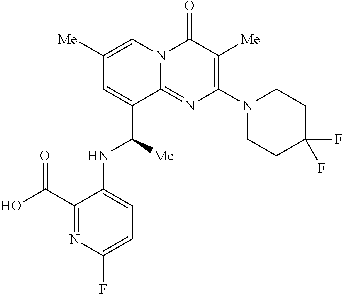 [SMILES: Cc1cc([C@@H](C)Nc2ccc(F)nc2C(=O)O)c2nc(N3CCC(F)(F)CC3)c(C)c(=O)n2c1] | (R)-3-((1-(2-(4,4- difluoropiperidin-1-yl)-3,7- dimethyl-4-oxo-4H-pyrido[1,2- a]pyrimidin-9-yl)ethyl)amino)-6- fluoropicolinic acid |
| 10-106 | 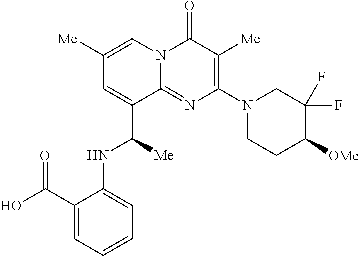 [SMILES: CO[C@H]1CCN(c2nc3c([C@@H](C)Nc4ccccc4C(=O)O)cc(C)cn3c(=O)c2C)CC1(F)F] | 2-(((R)-1-(2-((S)-3,3- difluoro-4-methoxypiperidin-1-yl)- 3,7-dimethyl-4-oxo-4H-pyrido[1,2- a]pyrimidin-9- yl)ethyl)amino)benzoic acid |
| 10-106 | 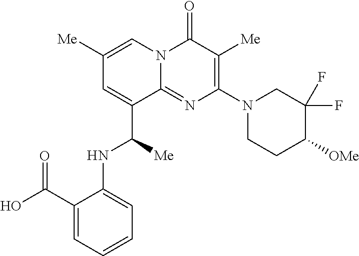 [SMILES: CO[C@@H]1CCN(c2nc3c([C@@H](C)Nc4ccccc4C(=O)O)cc(C)cn3c(=O)c2C)CC1(F)F] | 2-(((R)-1-(2-((R)-3,3- difluoro-4-methoxypiperidin-1-yl)- 3,7-dimethyl-4-oxo-4H-pyrido[1,2- a]pyrimidin-9- yl)ethyl)amino)benzoic acid |
| 10-107 | 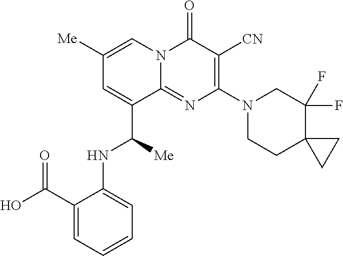 [SMILES: Cc1cc([C@@H](C)Nc2ccccc2C(=O)O)c2nc(N3CCC4(CC4)C(F)(F)C3)c(C#N)c(=O)n2c1] | (R)-2-((1-(2-(4,4-difluoro-6- azaspiro[2.5]octan-6-yl)-3,R7- dimethyl-4-oxo-4H-pyrido[1,2- a]pyrimidin-9- yl)ethyl)amino)benzoic acid |
| 10-108 | 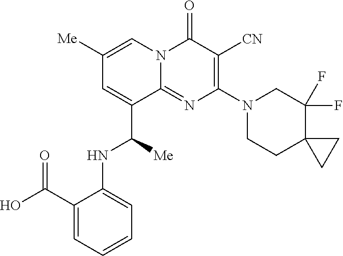 [SMILES: Cc1cc([C@@H](C)Nc2ccccc2C(=O)O)c2nc(N3CCC4(CC4)C(F)(F)C3)c(C#N)c(=O)n2c1] | (R)-2-((1-(3-cyano-2-(4,4- difluoro-6-azaspiro[2.5]octan-6- yl)-7-methyl-4-oxo-4H-pyrido[1,2- a]pyrimidin-9- yl)ethyl)amino)benzoic acid |
| 10-109 | 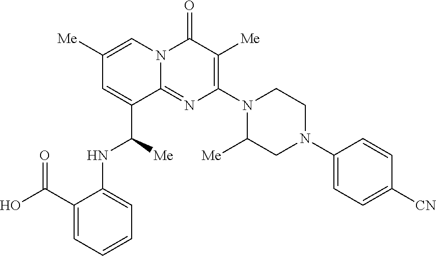 [SMILES: Cc1cc([C@@H](C)Nc2ccccc2C(=O)O)c2nc(N3CCN(c4ccc(C#N)cc4)CC3C)c(C)c(=O)n2c1] | 2-(((1R)-1-(3-cyano-2-(4-(4- cyanophenyl)-2-methylpiperazin- 1-yl)-7-methyl-4-oxo-4H- pyrido[1,2-a]pyrimidin-9- yl)ethyl)amino)benzoic acid |
| 10-110 | 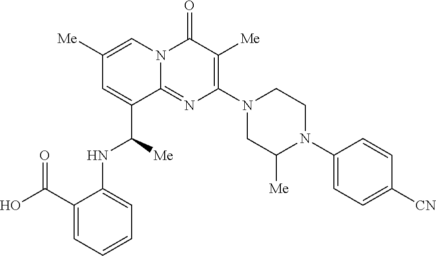 [SMILES: Cc1cc([C@@H](C)Nc2ccccc2C(=O)O)c2nc(N3CCN(c4ccc(C#N)cc4)C(C)C3)c(C)c(=O)n2c1] | 2-(((1R)-1-(2-(4-(4- cyanophenyl)-3-methylpiperazin- 1-yl)-3,7-dimethyl-4-oxo-4H- pyrido[1,2-a]pyrimidin-9- yl)ethyl)amino)benzoic acid |
| 10-111 | 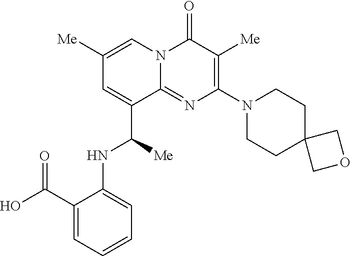 [SMILES: Cc1cc([C@@H](C)Nc2ccccc2C(=O)O)c2nc(N3CCC4(CC3)COC4)c(C)c(=O)n2c1] | (R)-2-((1-(3,7-dimethyl-4- oxo-2-(2-oxa-7- azaspiro[3.5]nonan-7-yl)-4H- pyrido[1,2-a]pyrimidin-9- yl)ethyl)amino)benzoic acid |
| 10-112 | 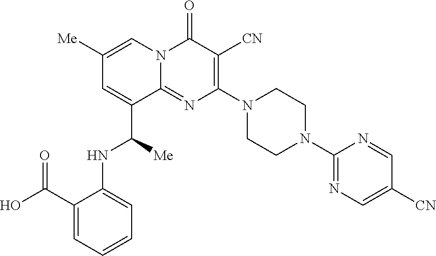 [SMILES: Cc1cc([C@@H](C)Nc2ccccc2C(=O)O)c2nc(N3CCN(c4ncc(C#N)cn4)CC3)c(C#N)c(=O)n2c1] | (R)-2-((1-(3-cyano-2-(4-(5- cyanopyrimidin-2-yl)piperazin-1- yl)-7-methyl-4-oxo-4H-pyrido[1,2- a]pyrimidin-9- yl)ethyl)amino)benzoic acid |
| 10-113 | 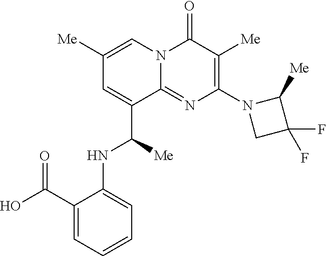 [SMILES: Cc1cc([C@@H](C)Nc2ccccc2C(=O)O)c2nc(N3CC(F)(F)[C@@H]3C)c(C)c(=O)n2c1] | 2-(((R)-1-(2-((S)-3,3- difluoro-2-methylazetidin-1-yl)- 3,7-dimethyl-4-oxo-4H-pyrido[1,2- a]pyrimidin-9- yl)ethyl)amino)benzoic acid |
| 10-113 | 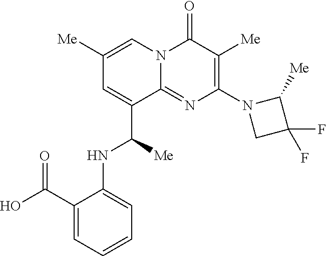 [SMILES: Cc1cc([C@@H](C)Nc2ccccc2C(=O)O)c2nc(N3CC(F)(F)[C@H]3C)c(C)c(=O)n2c1] | 2-(((R)-1-(2-((R)-3,3- difluoro-2-methylazetidin-1-yl)- 3,7-dimethyl-4-oxo-4H-pyrido[1,2- a]pyrimidin-9- yl)ethyl)amino)benzoic acid |
| 10-114 | 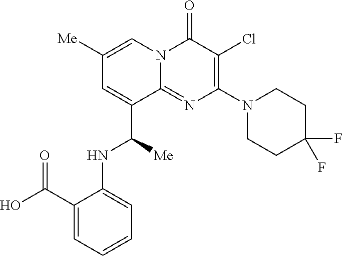 [SMILES: Cc1cc([C@@H](C)Nc2ccccc2C(=O)O)c2nc(N3CCC(F)(F)CC3)c(Cl)c(=O)n2c1] | (R)-2-((1-(3-chloro-2-(4,4- difluoropiperidin-1-yl)-7-methyl-4- oxo-4H-pyrido[1,2-a]pyrimidin-9- yl)ethyl)amino)benzoic acid |
| 10-115 | 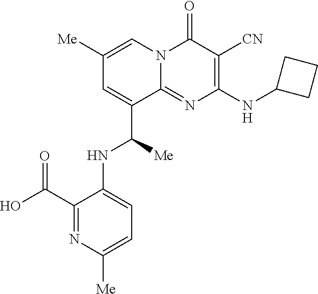 [SMILES: Cc1cc([C@@H](C)Nc2ccc(C)nc2C(=O)O)c2nc(NC3CCC3)c(C#N)c(=O)n2c1] | (R)-3-((1-(3-cyano-2- (cyclobutylamino)-7-methyl-4-oxo- 4H-pyrido[1,2-a]pyrimidin-9- yl)ethyl)amino)-6-methylpicolinic acid |
| 10-116 | 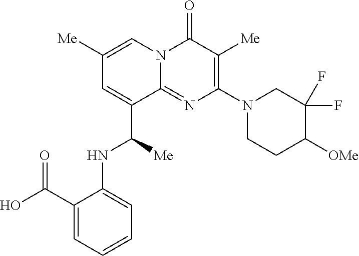 [SMILES: COC1CCN(c2nc3c([C@@H](C)Nc4ccccc4C(=O)O)cc(C)cn3c(=O)c2C)CC1(F)F] | 2-(((1R)-1-(2-(3,3-difluoro- 4-methoxypiperidin-1-yl)-3,7- dimethyl-4-oxo-4H-pyrido[1,2- a]pyrimidin-9- yl)ethyl)amino)benzoic acid |
| 10-117 | 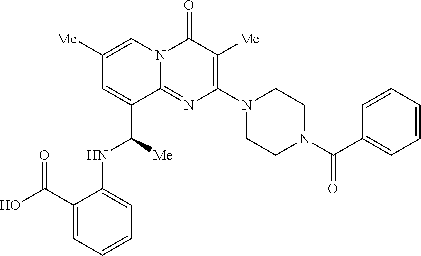 [SMILES: Cc1cc([C@@H](C)Nc2ccccc2C(=O)O)c2nc(N3CCN(C(=O)c4ccccc4)CC3)c(C)c(=O)n2c1] | (R)-2-((1-(2-(4- benzoylpiperazin-1-yl)-3,7- dimethyl-4-oxo-4H-pyrido[1,2- a]pyrimidin-9- yl)ethyl)amino)benzoic acid |
| 10-118 | 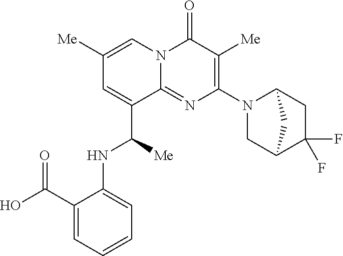 [SMILES: Cc1cc([C@@H](C)Nc2ccccc2C(=O)O)c2nc(N3C[C@H]4C[C@@H]3CC4(F)F)c(C)c(=O)n2c1] | 2-(((R)-1-(2-((1R,4R)-5,5- difluoro-2- azabicyclo[2.2.1]heptan-2-yl)-3,7- dimethyl-4-oxo-4H-pyrido[1,2- a]pyrimidin-9- yl)ethyl)amino)benzoic acid |
| 10-118 | 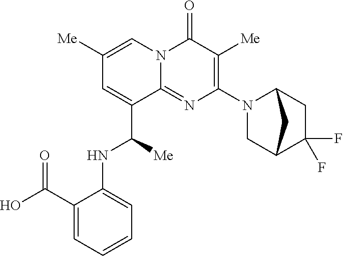 [SMILES: Cc1cc([C@@H](C)Nc2ccccc2C(=O)O)c2nc(N3C[C@@H]4C[C@H]3CC4(F)F)c(C)c(=O)n2c1] | 2-(((R)-1-(2-((1S,4S)-5,5- difluoro-2- azabicyclo[2.2.1]heptan-2-yl)-3,7- dimethyl-4-oxo-4H-pyrido[1,2- a]pyrimidin-9- yl)ethyl)amino)benzoic acid |
| 10-119 | 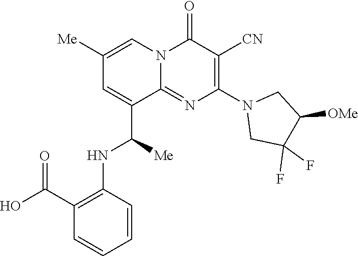 [SMILES: CO[C@@H]1CN(c2nc3c([C@@H](C)Nc4ccccc4C(=O)O)cc(C)cn3c(=O)c2C#N)CC1(F)F] | 2-(((R)-1-(3-cyano-2-((R)- 3,3-difluoro-4-methoxypyrrolidin- 1-yl)-7-methyl-4-oxo-4H- pyrido[1,2-a]pyrimidin-9- yl)ethyl)amino)benzoic acid |
| 10-119 | 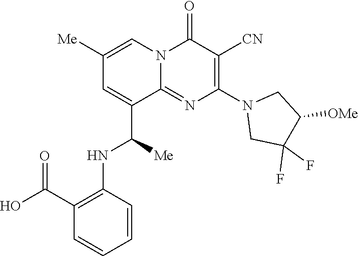 [SMILES: CO[C@H]1CN(c2nc3c([C@@H](C)Nc4ccccc4C(=O)O)cc(C)cn3c(=O)c2C#N)CC1(F)F] | 2-(((R)-1-(3-cyano-2-((S)- 3,3-difluoro-4-methoxypyrrolidin- 1-yl)-7-methyl-4-oxo-4H- pyrido[1,2-a]pyrimidin-9- yl)ethyl)amino)benzoic acid |
| 10-120 | 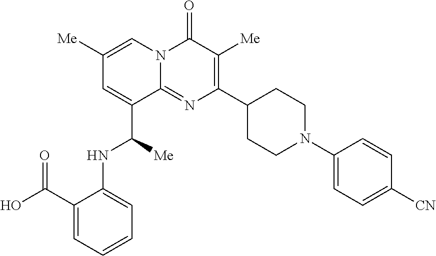 [SMILES: Cc1cc([C@@H](C)Nc2ccccc2C(=O)O)c2nc(C3CCN(c4ccc(C#N)cc4)CC3)c(C)c(=O)n2c1] | (R)-2-((1-(2-(1-(4- cyanophenyl)piperidin-4-yl)-3,7- dimethyl-4-oxo-4H-pyrido[1,2- a]pyrimidin-9- yl)ethyl)amino)benzoic acid |
| 10-121 | 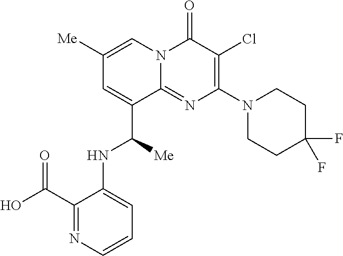 [SMILES: Cc1cc([C@@H](C)Nc2cccnc2C(=O)O)c2nc(N3CCC(F)(F)CC3)c(Cl)c(=O)n2c1] | (R)-3-((1-(3-cyano-2-(4,4- difluoropiperidin-1-yl)-7-methyl-4- oxo-4H-pyrido[1,2-a]pyrimidin-9- yl)ethyl)amino)picolinic acid |
| 10-122 | 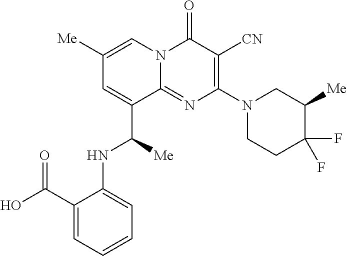 [SMILES: Cc1cc([C@@H](C)Nc2ccccc2C(=O)O)c2nc(N3CCC(F)(F)[C@H](C)C3)c(C#N)c(=O)n2c1] | 2-(((R)-1-(3-cyano-2-((R)- 4,4-difluoro-3-methylpiperidin-1- yl)-7-methyl-4-oxo-4H-pyrido[1,2- a]pyrimidin-9- yl)ethyl)amino)benzoic acid |
| 10-122 | 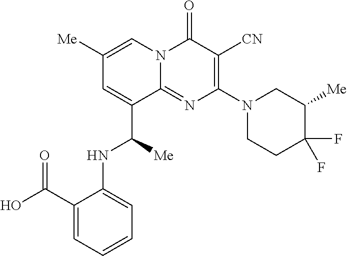 [SMILES: Cc1cc([C@@H](C)Nc2ccccc2C(=O)O)c2nc(N3CCC(F)(F)[C@@H](C)C3)c(C#N)c(=O)n2c1] | 2-(((R)-1-(3-cyano-2-((S)- 4,4-difluoro-3-methylpiperidin-1- yl)-7-methyl-4-oxo-4H-pyrido[1,2- a]pyrimidin-9- yl)ethyl)amino)benzoic acid |
| 10-123 | 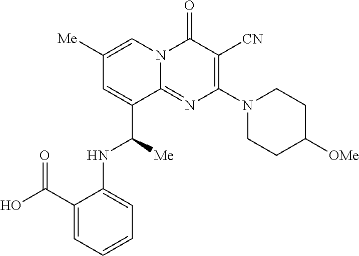 [SMILES: COC1CCN(c2nc3c([C@@H](C)Nc4ccccc4C(=O)O)cc(C)cn3c(=O)c2C#N)CC1] | (R)-2-((1-(3-cyano-2-(4- methoxypiperidin-1-yl)-7-methyl- 4-oxo-4H-pyrido[1,2-a]pyrimidin- 9-yl)ethyl)amino)benzoic acid |
| 10-124 | 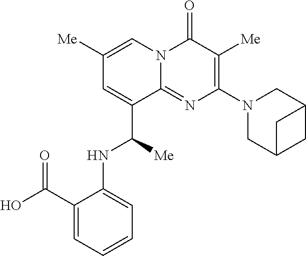 [SMILES: Cc1cc([C@@H](C)Nc2ccccc2C(=O)O)c2nc(N3CC4CC(C4)C3)c(C)c(=O)n2c1] | (R)-2-((1-(2-(3- azabicyclo[3.1.1]heptan-3-yl)-3,7- dimethyl-4-oxo-4H-pyrido[1,2- a]pyrimidin-9- yl)ethyl)amino)benzoic acid |
| 10-125 | 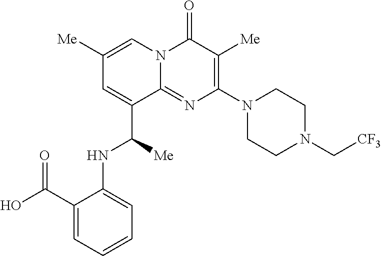 [SMILES: Cc1cc([C@@H](C)Nc2ccccc2C(=O)O)c2nc(N3CCN(CC(F)(F)F)CC3)c(C)c(=O)n2c1] | (R)-2-((1-(3,7-dimethyl-4- oxo-2-(4-(2,2,2- trifluoroethyl)piperazin-1-yl)-4H- pyrido[1,2-a]pyrimidin-9- yl)ethyl)amino)benzoic acid |
| 10-126 | 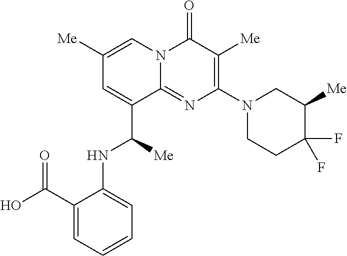 [SMILES: Cc1cc([C@@H](C)Nc2ccccc2C(=O)O)c2nc(N3CCC(F)(F)[C@H](C)C3)c(C)c(=O)n2c1] | 2-(((R)-1-(2-((R)-4,4- difluoro-3-methylpiperidin-1-yl)- 3,7-dimethyl-4-oxo-4H-pyrido[1,2- a]pyrimidin-9- yl)ethyl)amino)benzoic acid |
| 10-127 | 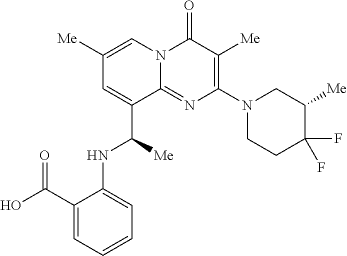 [SMILES: Cc1cc([C@@H](C)Nc2ccccc2C(=O)O)c2nc(N3CCC(F)(F)[C@@H](C)C3)c(C)c(=O)n2c1] | 2-(((R)-1-(2-((S)-4,4- difluoro-3-methylpiperidin-1-yl)- 3,7-dimethyl-4-oxo-4H-pyrido[1,2- a]pyrimidin-9- yl)ethyl)amino)benzoic acid |
| 10-128 | 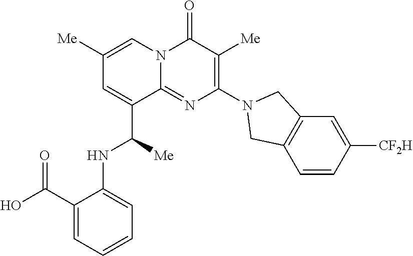 [SMILES: Cc1cc([C@@H](C)Nc2ccccc2C(=O)O)c2nc(N3Cc4ccc(C(F)F)cc4C3)c(C)c(=O)n2c1] | (R)-2-((1-(2-(5- (difluoromethyl)isoindolin-2-yl)- 3,7-dimethyl-4-oxo-4H-pyrido[1,2- a]pyrimidin-9- yl)ethyl)amino)benzoic acid |
| 10-129 | 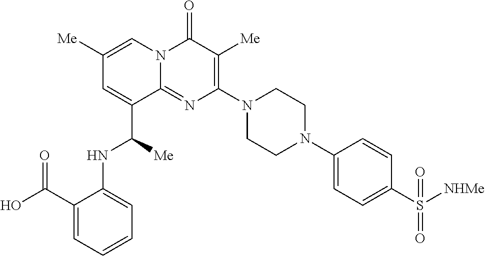 [SMILES: CNS(=O)(=O)c1ccc(N2CCN(c3nc4c([C@@H](C)Nc5ccccc5C(=O)O)cc(C)cn4c(=O)c3C)CC2)cc1] | (R)-2-((1-(3,7-dimethyl-2-(4- (4-(N- methylsulfamoyl)phenyl)piperazin- 1-yl)-4-oxo-4H-pyrido[1,2- a]pyrimidin-9- yl)ethyl)amino)benzoic acid |
| 10-130 | 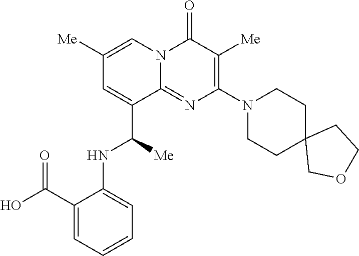 [SMILES: Cc1cc([C@@H](C)Nc2ccccc2C(=O)O)c2nc(N3CCC4(CCOC4)CC3)c(C)c(=O)n2c1] | (R)-2-((1-(3,7-dimethyl-4- oxo-2-(2-oxa-8- azaspiro[4.5]decan-8-yl)-4H- pyrido[1,2-a]pyrimidin-9- yl)ethyl)amino)benzoic acid |
| 10-131 | 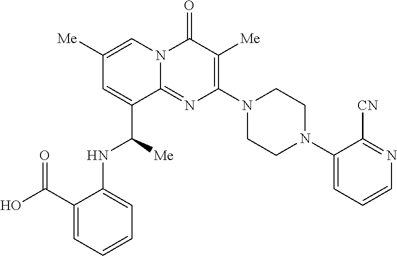 [SMILES: Cc1cc([C@@H](C)Nc2ccccc2C(=O)O)c2nc(N3CCN(c4cccnc4C#N)CC3)c(C)c(=O)n2c1] | (R)-2-((1-(2-(4-(2- cyanopyridin-3-yl)piperazin-1-yl)- 3,7-dimethyl-4-oxo-4H-pyrido[1,2- a]pyrimidin-9- yl)ethyl)amino)benzoic acid |
| 10-132 | 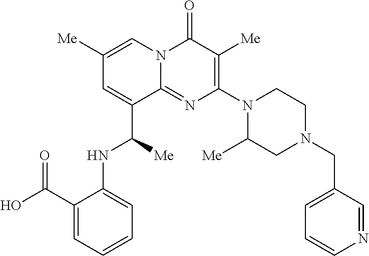 [SMILES: Cc1cc([C@@H](C)Nc2ccccc2C(=O)O)c2nc(N3CCN(Cc4cccnc4)CC3C)c(C)c(=O)n2c1] | 2-(((1R)-1-(3,7-dimethyl-2- (2-methyl-4-(pyridin-3- ylmethyl)piperazin-1-yl)-4-oxo-4H- pyrido[1,2-a]pyrimidin-9- yl)ethyl)amino)benzoic acid |
| 10-133 | 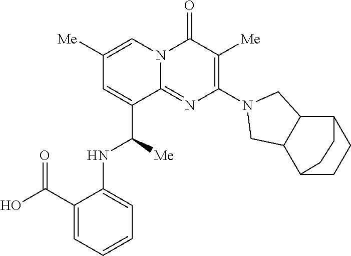 [SMILES: Cc1cc([C@@H](C)Nc2ccccc2C(=O)O)c2nc(N3CC4C5CCC(CC5)C4C3)c(C)c(=O)n2c1] | 2-(((1R)-1-(3,7-dimethyl-2- (octahydro-2H-4,7-ethanoisoindol- 2-yl)-4-oxo-4H-pyrido[1,2- a]pyrimidin-9- yl)ethyl)amino)benzoic acid |
| 10-134 | 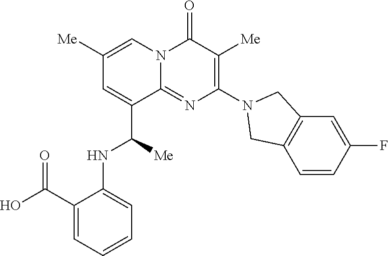 [SMILES: Cc1cc([C@@H](C)Nc2ccccc2C(=O)O)c2nc(N3Cc4ccc(F)cc4C3)c(C)c(=O)n2c1] | (R)-2-((1-(2-(5- fluoroisoindolin-2-yl)-3,7-dimethyl- 4-oxo-4H-pyrido[1,2-a]pyrimidin- 9-yl)ethyl)amino)benzoic acid |
| 10-135 | 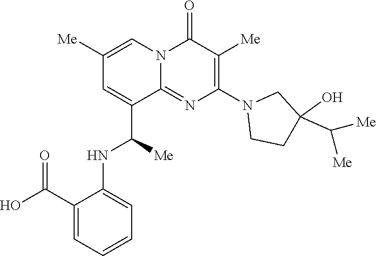 [SMILES: Cc1cc([C@@H](C)Nc2ccccc2C(=O)O)c2nc(N3CCC(O)(C(C)C)C3)c(C)c(=O)n2c1] | 2-(((1R)-1-(2-(3-hydroxy-3- isopropylpyrrolidin-1-yl)-3,7- dimethyl-4-oxo-4H-pyrido[1,2- a]pyrimidin-9- yl)ethyl)amino)benzoic acid |
| 10-136 | 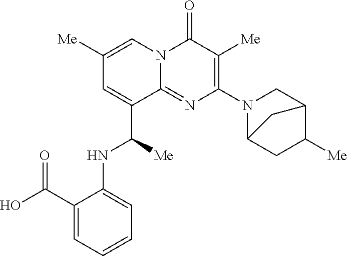 [SMILES: Cc1cc([C@@H](C)Nc2ccccc2C(=O)O)c2nc(N3CC4CC3CC4C)c(C)c(=O)n2c1] | 2-(((1R)-1-(3,7-dimethyl-2- (5-methyl-2- azabicyclo[2.2.1]heptan-2-yl)-4- oxo-4H-pyrido[1,2-a]pyrimidin-9- yl)ethyl)amino)benzoic acid |
| 10-137 | 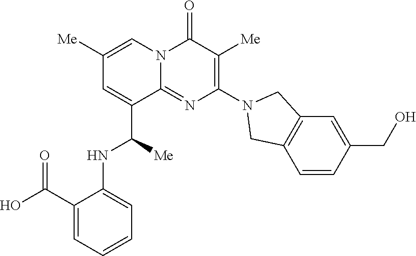 [SMILES: Cc1cc([C@@H](C)Nc2ccccc2C(=O)O)c2nc(N3Cc4ccc(CO)cc4C3)c(C)c(=O)n2c1] | (R)-2-((1-(2-(5- (hydroxymethyl)isoindolin-2-yl)- 3,7-dimethyl-4-oxo-4H-pyrido[1,2- a]pyrimidin-9- yl)ethyl)amino)benzoic acid |
| 10-138 | 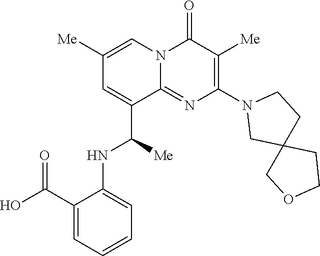 [SMILES: Cc1cc([C@@H](C)Nc2ccccc2C(=O)O)c2nc(N3CCC4(CCOC4)C3)c(C)c(=O)n2c1] | 2-(((1R)-1-(3,7-dimethyl-4- oxo-2-(2-oxa-7- azaspiro[4.4]nonan-7-yl)-4H- pyrido[1,2-a]pyrimidin-9- yl)ethyl)amino)benzoic acid |
| 10-139 | 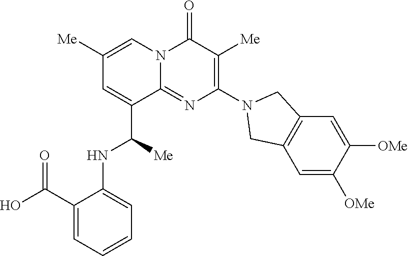 [SMILES: COc1cc2c(cc1OC)CN(c1nc3c([C@@H](C)Nc4ccccc4C(=O)O)cc(C)cn3c(=O)c1C)C2] | (R)-2-((1-(2-(5,6- dimethoxyisoindolin-2-yl)-3,7- dimethyl-4-oxo-4H-pyrido[1,2- a]pyrimidin-9- yl)ethyl)amino)benzoic acid |
| 10-140 | 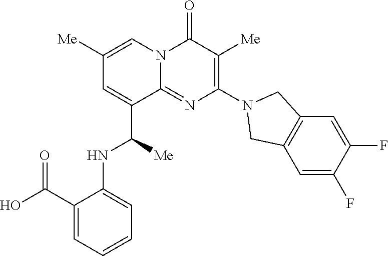 [SMILES: Cc1cc([C@@H](C)Nc2ccccc2C(=O)O)c2nc(N3Cc4cc(F)c(F)cc4C3)c(C)c(=O)n2c1] | (R)-2-((1-(2-(5,6- difluoroisoindolin-2-yl)-3,7- dimethyl-4-oxo-4H-pyrido[1,2- a]pyrimidin-9- yl)ethyl)amino)benzoic acid |
| 10-141 | 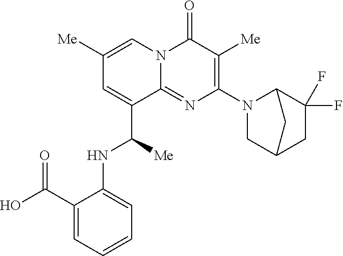 [SMILES: Cc1cc([C@@H](C)Nc2ccccc2C(=O)O)c2nc(N3CC4CC3C(F)(F)C4)c(C)c(=O)n2c1] | 2-(((1R)-1-(2-(6,6-difluoro- 2-azabicyclo[2.2.1]heptan-2-yl)- 3,7-dimethyl-4-oxo-4H-pyrido[1,2- a]pyrimidin-9- yl)ethyl)amino)benzoic acid |
| 10-142 | 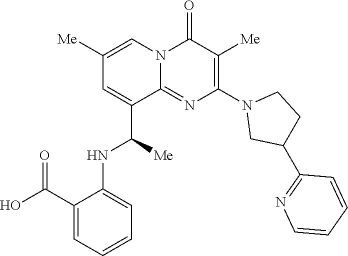 [SMILES: Cc1cc([C@@H](C)Nc2ccccc2C(=O)O)c2nc(N3CCC(c4ccccn4)C3)c(C)c(=O)n2c1] | 2-(((1R)-1-(3,7-dimethyl-4- oxo-2-(3-(pyridin-2-yl)pyrrolidin-1- yl)-4H-pyrido[1,2-a]pyrimidin-9- yl)ethyl)amino)benzoic acid |
| 10-143 | 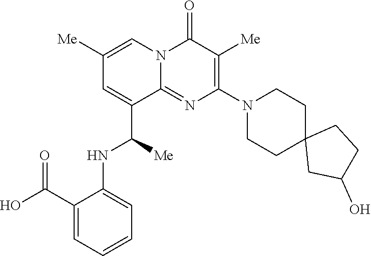 [SMILES: Cc1cc([C@@H](C)Nc2ccccc2C(=O)O)c2nc(N3CCC4(CCC(O)C4)CC3)c(C)c(=O)n2c1] | 2-(((1R)-1-(2-(2-hydroxy-8- azaspiro[4.5]decan-8-yl)-3,7- dimethyl-4-oxo-4H-pyrido[1,2- a]pyrimidin-9- yl)ethyl)amino)benzoic acid |
| 10-144 | 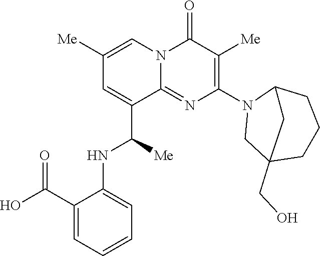 [SMILES: Cc1cc([C@@H](C)Nc2ccccc2C(=O)O)c2nc(N3CC4(CO)CCCC3C4)c(C)c(=O)n2c1] | 2-(((1R)-1-(2-(1- (hydroxymethyl)-6- azabicyclo[3.2.1]octan-6-yl)-3,7- dimethyl-4-oxo-4H-pyrido[1,2- a]pyrimidin-9- yl)ethyl)amino)benzoic acid |
| 10-145 | 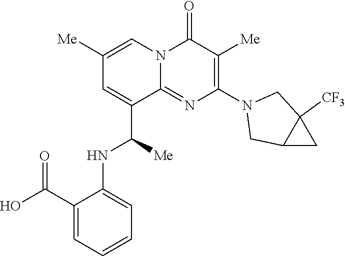 [SMILES: Cc1cc([C@@H](C)Nc2ccccc2C(=O)O)c2nc(N3CC4CC4(C(F)(F)F)C3)c(C)c(=O)n2c1] | 2-(((1R)-1-(3,7-dimethyl-4- oxo-2-(1-(trifluoromethyl)-3- azabicyclo[3.1.0]hexan-3-yl)-4H- pyrido[1,2-a]pyrimidin-9- yl)ethyl)amino)benzoic acid |
| 10-146 | 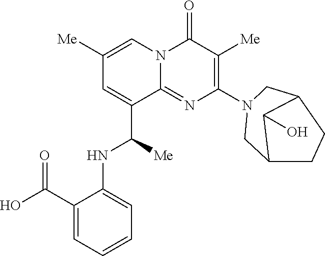 [SMILES: Cc1cc([C@@H](C)Nc2ccccc2C(=O)O)c2nc(N3CC4CCC(C3)C4O)c(C)c(=O)n2c1] | 2-(((1R)-1-(2-(8-hydroxy-3- azabicyclo[3.2.1]octan-3-yl)-3,7- dimethyl-4-oxo-4H-pyrido[1,2- a]pyrimidin-9- yl)ethyl)amino)benzoic acid |
| 10-147 | 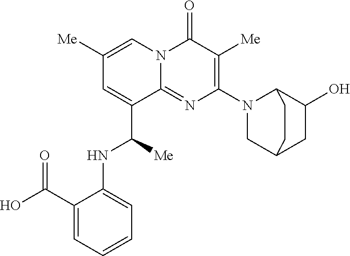 [SMILES: Cc1cc([C@@H](C)Nc2ccccc2C(=O)O)c2nc(N3CC4CCC3C(O)C4)c(C)c(=O)n2c1] | 2-(((1R)-1-(2-(6-hydroxy-2- azabicyclo[2.2.2]octan-2-yl)-3,7- dimethyl-4-oxo-4H-pyrido[1,2- a]pyrimidin-9- yl)ethyl)amino)benzoic acid; |
| 10-148 | 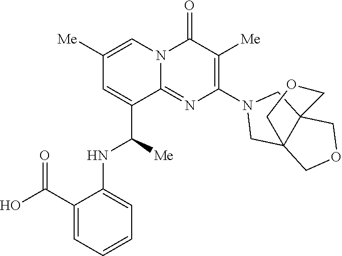 [SMILES: Cc1cc([C@@H](C)Nc2ccccc2C(=O)O)c2nc(N3CC45COCC4(COC5)C3)c(C)c(=O)n2c1] | 2-(((R)-1-(2-((3aS,6aS)- 1H,3H,4H-3a,6a- (methanooxymethano)furo[3,4- c]pyrrol-5(6H)-yl)-3,7-dimethyl-4- oxo-4H-pyrido[1,2-a]pyrimidin-9- yl)ethyl)amino)benzoic acid |

or a pharmaceutically acceptable salt of one of the foregoing compounds.

In another embodiment, the compound of Formula I is selected from:

| | |
|---|---|
| 11-1 | (R)-2-((1-(2-(4-(2-chlorophenyl)piperazin-1-yl)-3,7-dimethyl-4-oxo-4H- |
| | pyrido[1,2-a]pyrimidin-9-yl)ethyl)amino)benzoic acid |
| 11-2 | 2-(((1R)-1-(2-(4,4-difluoro-3-methylpiperidin-1-yl)-3,7-dimethyl-4-oxo- |
| | 4H-pyrido[1,2-a]pyrimidin-9-yl)ethyl)amino)benzoic acid |
| 11-3 | 2-(((1R)-1-(2-(4,4-difluoro-3-(hydroxymethyl)piperidin-1-yl)-3,7- |
| | dimethyl-4-oxo-4H-pyrido[1,2-a]pyrimidin-9-yl)ethyl)amino)benzoic acid |
| 11-4 | 2-(((1R)-1-(2-(8,8-difluoro-3-azabicyclo[3.2.1]octan-3-yl)-3,7-dimethyl- |
| | 4-oxo-4H-pyrido[1,2-a]pyrimidin-9-yl)ethyl)amino)benzoic acid |
| 11-5 | 2-(((1R)-1-(3,7-dimethyl-4-oxo-2-(3,4,4-trifluoropiperidin-1-yl)-4H- |
| | pyrido[1,2-a]pyrimidin-9-yl)ethyl)amino)benzoic acid |
| 11-6 | 2-(((1R)-1-(2-(9,9-difluoro-3-oxa-7-azabicyclo[3.3.1]nonan-7-yl)-3,7- |
| | dimethyl-4-oxo-4H-pyrido[1,2-a]pyrimidin-9-yl)ethyl)amino)benzoic acid |
| 11-7 | 2-(((1R)-1-(3,7-dimethyl-4-oxo-2-(7,7,9,9-tetrafluoro-3- |
| | azabicyclo[3.3.1]nonan-3-yl)-4H-pyrido[1,2-a]pyrimidin-9- |
| | yl)ethyl)amino)benzoic acid |
| 11-8 | 2-(((1R)-1-(2-(9,9-difluoro-3-azabicyclo[3.3.1]nonan-3-yl)-3,7- |
| | dimethyl-4-oxo-4H-pyrido[1,2-a]pyrimidin-9-yl)ethyl)amino)benzoic acid |
| 11-9 | 2-(((1R)-1-(2-(9,9-difluoro-1-(hydroxymethyl)-3- |
| | azabicyclo[3.3.1]nonan-3-yl)-3,7-dimethyl-4-oxo-4H-pyrido[1,2-a]pyrimidin- |
| | 9-yl)ethyl)amino)benzoic acid |
| 11-10 | 2-(((R)-1-(2-((3R,5R)-4,4-difluoro-3,5-dimethylpiperidin-1-yl)-3,7- |
| | dimethyl-4-oxo-4H-pyrido[1,2-a]pyrimidin-9-yl)ethyl)amino)benzoic acid |
| | And |
| | 2-(((R)-1-(2-((3S,5S)-4,4-difluoro-3,5-dimethylpiperidin-1-yl)-3,7- |
| | dimethyl-4-oxo-4H-pyrido[1,2-a]pyrimidin-9-yl)ethyl)amino)benzoic acid |
| 11-11 | 2-(((1R)-1-(2-(5,5-difluoro-2-azabicyclo[2.2.2]octan-2-yl)-3,7-dimethyl- |
| | 4-oxo-4H-pyrido[1,2-a]pyrimidin-9-yl)ethyl)amino)benzoic acid |
| 11-12 | 2-(((R)-1-(2-((3R,5S)-4,4-difluoro-3,5-dimethylpiperidin-1-yl)-3,7- |
| | dimethyl-4-oxo-4H-pyrido[1,2-a]pyrimidin-9-yl)ethyl)amino)benzoic acid |
| | And |
| | 2-(((R)-1-(2-((3S,5R)-4,4-difluoro-3,5-dimethylpiperidin-1-yl)-3,7- |
| | dimethyl-4-oxo-4H-pyrido[1,2-a]pyrimidin-9-yl)ethyl)amino)benzoic acid; |
| 11-13 | 2-(((1R)-1-(2-(7-(tert-butyl)-6,7-dihydropyrazolo[1,5-a]pyrimidin-4(5H)- |
| | yl)-3,7-dimethyl-4-oxo-4H-pyrido[1,2-a]pyrimidin-9-yl)ethyl)amino)benzoic |
| | acid |
| 11-14 | 2-(((1R)-1-(3,7-dimethyl-4-oxo-2-(3-phenylpyrrolidin-1-yl)-4H- |
| | pyrido[1,2-a]pyrimidin-9-yl)ethyl)amino)benzoic acid |
| 11-15 | (R)-2-((1-(3,7-dimethyl-2-(2-methyl-4,6-dihydro-5H-pyrrolo[3,4- |
| | d]thiazol-5-yl)-4-oxo-4H-pyrido[1,2-a]pyrimidin-9-yl)ethyl)amino)benzoic acid |
| 11-16 | (R)-2-((1-(3,7-dimethyl-4-oxo-2-(3-(pyridin-2-yl)azetidin-1-yl)-4H- |
| | pyrido[1,2-a]pyrimidin-9-yl)ethyl)amino)benzoic acid |
| 11-17 | (R)-2-((1-(3,7-dimethyl-2-(5-methylisoindolin-2-yl)-4-oxo-4H- |
| | pyrido[1,2-a]pyrimidin-9-yl)ethyl)amino)benzoic acid |
| 11-18 | (R)-2-((1-(3,7-dimethyl-4-oxo-2-(3-phenylazetidin-1-yl)-4H-pyrido[1,2- |
| | a]pyrimidin-9-yl)ethyl)amino)benzoic acid |
| 11-19 | 2-(((1R)-1-(2-(1-(hydroxymethyl)isoindolin-2-yl)-3,7-dimethyl-4-oxo- |
| | 4H-pyrido[1,2-a]pyrimidin-9-yl)ethyl)amino)benzoic acid |
| 11-20 | (R)-2-((1-(2-(4-cyclobutylpiperidin-1-yl)-3,7-dimethyl-4-oxo-4H- |
| | pyrido[1,2-a]pyrimidin-9-yl)ethyl)amino)benzoic acid |
| 11-21 | (R)-2-((1-(3,7-dimethyl-4-oxo-2-(3-(pyridin-3-yl)azetidin-1-yl)-4H- |
| | pyrido[1,2-a]pyrimidin-9-yl)ethyl)amino)benzoic acid |
| 11-22 | 2-(((1R)-1-(2-(6-hydroxyhexahydro-3,5-methanocyclopenta[b]pyrrol- |
| | 1(2H)-yl)-3,7-dimethyl-4-oxo-4H-pyrido[1,2-a]pyrimidin-9- |
| | yl)ethyl)amino)benzoic acid |
| 11-23 | (R)-2-((1-(2-(4,4-difluoro-[1,4′-bipiperidin]-1′-yl)-3,7-dimethyl-4-oxo- |
| | 4H-pyrido[1,2-a]pyrimidin-9-yl)ethyl)amino)benzoic acid |
| 11-24 | 2-(((1R)-1-(2-(6,6-dimethyl-3-azabicyclo[3.2.0]heptan-3-yl)-3,7- |
| | dimethyl-4-oxo-4H-pyrido[1,2-a]pyrimidin-9-yl)ethyl)amino)benzoic acid |
| 11-25 | (R)-2-((1-(2-(4-cyclopentylpiperidin-1-yl)-3,7-dimethyl-4-oxo-4H- |
| | pyrido[1,2-a]pyrimidin-9-yl)ethyl)amino)benzoic acid |
| 11-26 | (R)-2-((1-(2-(2-azadispiro[3.1.3⁶.1⁴]decan-2-yl)-3,7-dimethyl-4-oxo-4H- |
| | pyrido[1,2-a]pyrimidin-9-yl)ethyl)amino)benzoic acid |
| 11-27 | 2-(((1R)-1-(2-(decahydro-2H-cycloocta[c]pyrrol-2-yl)-3,7-dimethyl-4- |
| | oxo-4H-pyrido[1,2-a]pyrimidin-9-yl)ethyl)amino)benzoic acid |
| 11-28 | 2-(((1R)-1-(2-(8-hydroxy-6-azaspiro[3.4]octan-6-yl)-3,7-dimethyl-4- |
| | oxo-4H-pyrido[1,2-a]pyrimidin-9-yl)ethyl)amino)benzoic acid |
| 11-29 | (R)-2-((1-(2-(2-azaadamantan-2-yl)-3,7-dimethyl-4-oxo-4H-pyrido[1,2- |
| | a]pyrimidin-9-yl)ethyl)amino)benzoic acid |
| 11-30 | 2-(((1R)-1-(2-(5-hydroxy-2-azabicyclo[2.2.2]octan-2-yl)-3,7-dimethyl-4- |
| | oxo-4H-pyrido[1,2-a]pyrimidin-9-yl)ethyl)amino)benzoic acid |
| 11-31 | 2-(((1R)-1-(2-(3-hydroxyoctahydro-1H-indol-1-yl)-3,7-dimethyl-4-oxo- |
| | 4H-pyrido[1,2-a]pyrimidin-9-yl)ethyl)amino)benzoic acid |
| 11-32 | 2-(((1R)-1-(3,7-dimethyl-4-oxo-2-(5-phenyl-2-azabicyclo[2.2.1]heptan- |
| | 2-yl)-4H-pyrido[1,2-a]pyrimidin-9-yl)ethyl)amino)benzoic acid |
| 11-33 | 2-(((1R)-1-(3,7-dimethyl-4-oxo-2-(8-azatricyclo[4.3.0.0^{2,5}]nonan-8-yl)- |
| | 4H-pyrido[1,2-a]pyrimidin-9-yl)ethyl)amino)benzoic acid |
| 11-34 | 2-(((1R)-1-(2-(6-hydroxy-6-methyl-3-azabicyclo[3.1.1]heptan-3-yl)-3,7- |
| | dimethyl-4-oxo-4H-pyrido[1,2-a]pyrimidin-9-yl)ethyl)amino)benzoic acid |
| 11-35 | 2-(((1R)-1-(2-(8-hydroxyoctahydro-2H-4,7-methanoisoindol-2-yl)-3,7- |
| | dimethyl-4-oxo-4H-pyrido[1,2-a]pyrimidin-9-yl)ethyl)amino)benzoic acid |
| 11-36 | (R)-2-((1-(2-(4-(bicyclo[1.1.1]pentan-1-yl-3-d)piperidin-1-yl)-3,7- |
| | dimethyl-4-oxo-4H-pyrido[1,2-a]pyrimidin-9-yl)ethyl)amino)benzoic acid |
| 11-37 | (R)-2-((1-(2-(4-(2,6-dimethylphenyl)piperazin-1-yl)-3,7-dimethyl-4-oxo- |
| | 4H-pyrido[1,2-a]pyrimidin-9-yl)ethyl)amino)benzoic acid |
| 11-38 | (R)-2-((1-(2-(4-fluoroisoindolin-2-yl)-3,7-dimethyl-4-oxo-4H-pyrido[1,2- |
| | a]pyrimidin-9-yl)ethyl)amino)benzoic acid |
| 11-39 | (R)-2-((1-(2-(4-(4-methoxyphenyl)piperidin-1-yl)-3,7-dimethyl-4-oxo- |
| | 4H-pyrido[1,2-a]pyrimidin-9-yl)ethyl)amino)benzoic acid |
| 11-40 | 1-(2-(isoindolin-2-yl)-7-methyl-4-oxo-4H-pyrido[1,2-a]pyrimidin-9-yl)- |
| | 1,2,3,4-tetrahydroquinoline-3-carboxylic acid |
| 11-41 | 2-(4-(2-(isoindolin-2-yl)-7-methyl-4-oxo-4H-pyrido[1,2-a]pyrimidin-9- |
| | yl)-7-oxo-1,4-diazepan-1-yl)acetic acid |
| 11-42 | (R)-2-((1-(3-cyano-2-(4-(2,2-difluoroethyl)piperazin-1-yl)-7-methyl-4- |
| | oxo-4H-pyrido[1,2-a]pyrimidin-9-yl)ethyl)amino)benzoic acid |
| 11-43 | (S)-3-((1-(3-cyano-2-(4,4-difluoropiperidin-1-yl)-7-methyl-4-oxo-4H- |
| | pyrido[1,2-a]pyrimidin-9-yl)ethyl)amino)-6-fluoropicolinic acid |
| 11-44 | (R)-3-((1-(3-cyano-2-(4,4-difluoropiperidin-1-yl)-7-methyl-4-oxo-4H- |
| | pyrido[1,2-a]pyrimidin-9-yl)ethyl)amino)-6-fluoropicolinic acid |
| 11-45 | (R)-3-((1-(2-(4,4-difluoropiperidin-1-yl)-3,7-dimethyl-4-oxo-4H- |
| | pyrido[1,2-a]pyrimidin-9-yl)ethyl)amino)-6-fluoropicolinic acid |
| 11-46 | (R)-3-((1-(2-(4,4-difluoropiperidin-1-yl)-3,7-dimethyl-4-oxo-4H- |
| | pyrido[1,2-a]pyrimidin-9-yl)ethyl)amino)-6-methylpicolinic acid |
| 11-47 | 2-(((R)-1-(3-cyano-2-((3R,5R)-3,5-difluoropiperidin-1-yl)-7-methyl-4- |
| | oxo-4H-pyrido[1,2-a]pyrimidin-9-yl)ethyl)amino)benzoic acid |
| 11-48 | 2-(((R)-1-(3-cyano-2-((3R,5S)-3,5-difluoropiperidin-1-yl)-7-methyl-4- |
| | oxo-4H-pyrido[1,2-a]pyrimidin-9-yl)ethyl)amino)benzoic acid |
| 11-49 | 2-(((1R)-1-(3-cyano-7-methyl-4-oxo-2-((3,3,3-trifluoro-2- |
| | methylpropyl)amino)-4H-pyrido[1,2-a]pyrimidin-9-yl)ethyl)amino)benzoic |
| | acid |
| 11-50 | (R)-6-chloro-3-((1-(7-chloro-3-cyano-2-(4,4-difluoropiperidin-1-yl)-4- |
| | oxo-4H-pyrido[1,2-a]pyrimidin-9-yl)ethyl)amino)picolinic acid |
| 11-51 | 2-(((R)-1-(3-cyano-7-methyl-4-oxo-2-(((R)-1-(pyridin-3-yl)ethyl)amino)- |
| | 4H-pyrido[1,2-a]pyrimidin-9-yl)ethyl)amino)benzoic acid |
| 11-52 | 2-(((R)-1-(3-cyano-7-methyl-4-oxo-2-(((S)-1-(pyridin-3-yl)ethyl)amino)- |
| | 4H-pyrido[1,2-a]pyrimidin-9-yl)ethyl)amino)benzoic acid |
| 11-53 | (R)-2-((1-(2-(4-(1H-pyrazol-3-yl)piperidin-1-yl)-3-cyano-7-methyl-4- |
| | oxo-4H-pyrido[1,2-a]pyrimidin-9-yl)ethyl)amino)benzoic acid |
| 11-54 | (R)-2-((1-(2-(2,2-difluoro-7-azaspiro[3.5]nonan-7-yl)-3,7-dimethyl-4- |
| | oxo-4H-pyrido[1,2-a]pyrimidin-9-yl)ethyl)amino)benzoic acid |
| 11-55 | (R)-2-((1-(3-cyano-7-methyl-4-oxo-2-(1-oxa-7-azaspiro[3.5]nonan-7- |
| | yl)-4H-pyrido[1,2-a]pyrimidin-9-yl)ethyl)amino)benzoic acid |
| 11-56 | (R)-2-((1-(3,7-dimethyl-4-oxo-2-(1-oxa-7-azaspiro[3.5]nonan-7-yl)-4H- |
| | pyrido[1,2-a]pyrimidin-9-yl)ethyl)amino)benzoic acid |
| 11-57 | 2-(((R)-1-(2-((S)-4,4-difluoro-3-methoxypiperidin-1-yl)-3,7-dimethyl-4- |
| | oxo-4H-pyrido[1,2-a]pyrimidin-9-yl)ethyl)amino)benzoic acid |
| 11-58 | 2-(((R)-1-(2-((R)-4,4-difluoro-3-methoxypiperidin-1-yl)-3,7-dimethyl-4- |
| | oxo-4H-pyrido[1,2-a]pyrimidin-9-yl)ethyl)amino)benzoic acid |
| 11-59 | 2-(((R)-1-(2-((R)-3,3-difluoro-4-methylpiperidin-1-yl)-3,7-dimethyl-4- |
| | oxo-4H-pyrido[1,2-a]pyrimidin-9-yl)ethyl)amino)benzoic acid |
| 11-60 | 2-(((R)-1-(2-((S)-3,3-difluoro-4-methylpiperidin-1-yl)-3,7-dimethyl-4- |
| | oxo-4H-pyrido[1,2-a]pyrimidin-9-yl)ethyl)amino)benzoic acid |
| 11-61 | (R)-2-((1-(2-(7,7-difluoro-5-azaspiro[2.4]heptan-5-yl)-3,7-dimethyl-4- |
| | oxo-4H-pyrido[1,2-a]pyrimidin-9-yl)ethyl)amino)benzoic acid |
| 11-62 | (R)-2-((1-(3,7-dimethyl-4-oxo-2-(2-oxa-8-azaspiro[4.5]decan-8-yl)-4H- |
| | pyrido[1,2-a]pyrimidin-9-yl)ethyl)amino)benzoic acid |
| 11-63 | (R)-2-((1-(3,7-dimethyl-4-oxo-2-(1-oxa-8-azaspiro[4.5]decan-8-yl)-4H- |
| | pyrido[1,2-a]pyrimidin-9-yl)ethyl)amino)benzoic acid |
| 11-64 | 2-(((R)-1-(3-cyano-2-((R)-4,4-difluoro-3-methylpiperidin-1-yl)-7-methyl- |
| | 4-oxo-4H-pyrido[1,2-a]pyrimidin-9-yl)ethyl)amino)benzoic acid |
| 11-65 | 2-(((1R)-1-(2-(4,4-difluoro-3-methylpiperidin-1-yl)-3,7-dimethyl-4-oxo- |
| | 4H-pyrido[1,2-a]pyrimidin-9-yl)ethyl)amino)benzoic acid |
| 11-66 | 2-(((R)-1-(3-cyano-2-((R)-3,3-difluoro-4-methoxypiperidin-1-yl)-7- |
| | methyl-4-oxo-4H-pyrido[1,2-a]pyrimidin-9-yl)ethyl)amino)benzoic acid |
| 11-67 | 2-(((1R)-1-(3-cyano-2-(3,3-difluoro-4-methoxypiperidin-1-yl)-7-methyl- |
| | 4-oxo-4H-pyrido[1,2-a]pyrimidin-9-yl)ethyl)amino)benzoic acid |
| 11-68 | (R)-2-((1-(3-cyano-2-(4-cyano-4-methylpiperidin-1-yl)-7-methyl-4-oxo- |
| | 4H-pyrido[1,2-a]pyrimidin-9-yl)ethyl)amino)benzoic acid |
| 11-69 | (R)-2-((1-(2-(4-cyano-4-methylpiperidin-1-yl)-3,7-dimethyl-4-oxo-4H- |
| | pyrido[1,2-a]pyrimidin-9-yl)ethyl)amino)benzoic acid |
| 11-70 | (R)-2-((1-(3-cyano-7-methyl-4-oxo-2-(4-(trifluoromethyl)piperidin-1-yl)- |
| | 4H-pyrido[1,2-a]pyrimidin-9-yl)ethyl)amino)benzoic acid |
| 11-71 | (R)-2-((1-(3,7-dimethyl-4-oxo-2-(4-(trifluoromethyl)piperidin-1-yl)-4H- |
| | pyrido[1,2-a]pyrimidin-9-yl)ethyl)amino)benzoic acid |
| 11-72 | (R)-2-((1-(3-cyano-2-(4-cyanopiperidin-1-yl)-7-methyl-4-oxo-4H- |
| | pyrido[1,2-a]pyrimidin-9-yl)ethyl)amino)benzoic acid |
| 11-73 | (R)-2-((1-(2-(4-cyanopiperidin-1-yl)-3,7-dimethyl-4-oxo-4H-pyrido[1,2- |
| | a]pyrimidin-9-yl)ethyl)amino)benzoic acid |
| 11-74 | 2-(((R)-1-(2-((R)-4-(2-cyano-5-methylphenyl)-2-methylpiperazin-1-yl)- |
| | 3,7-dimethyl-4-oxo-4H-pyrido[1,2-a]pyrimidin-9-yl)ethyl)amino)benzoic acid |
| 11-75 | 2-(((R)-1-(2-((S)-4-(2-cyano-5-methylphenyl)-2-methylpiperazin-1-yl)- |
| | 3,7-dimethyl-4-oxo-4H-pyrido[1,2-a]pyrimidin-9-yl)ethyl)amino)benzoic acid |
| 11-76 | 2-(((R)-1-(3-cyano-2-((R)-4-(2-cyano-4-(trifluoromethyl)phenyl)-2- |
| | methylpiperazin-1-yl)-7-methyl-4-oxo-4H-pyrido[1,2-a]pyrimidin-9- |
| | yl)ethyl)amino)benzoic acid |
| 11-77 | 2-(((R)-1-(3-cyano-2-((S)-4-(2-cyano-4-(trifluoromethyl)phenyl)-2- |
| | methylpiperazin-1-yl)-7-methyl-4-oxo-4H-pyrido[1,2-a]pyrimidin-9- |
| | yl)ethyl)amino)benzoic acid |
| 11-78 | 2-(((1R)-1-(3-cyano-2-(4-(2-cyano-4-(trifluoromethyl)phenyl)-2- |
| | methylpiperazin-1-yl)-7-methyl-4-oxo-4H-pyrido[1,2-a]pyrimidin-9- |
| | yl)ethyl)amino)benzoic acid |
| 11-79 | 2-(((R)-1-(2-((R)-4-(2-cyano-4-(trifluoromethyl)phenyl)-2- |
| | methylpiperazin-1-yl)-3,7-dimethyl-4-oxo-4H-pyrido[1,2-a]pyrimidin-9- |
| | yl)ethyl)amino)benzoic acid |
| 11-80 | 2-(((R)-1-(2-((S)-4-(2-cyano-4-(trifluoromethyl)phenyl)-2- |
| | methylpiperazin-1-yl)-3,7-dimethyl-4-oxo-4H-pyrido[1,2-a]pyrimidin-9- |
| | yl)ethyl)amino)benzoic acid |
| 11-81 | 2-(((1R)-1-(2-(4-(2-cyano-4-(trifluoromethyl)phenyl)-2-methylpiperazin- |
| | 1-yl)-3,7-dimethyl-4-oxo-4H-pyrido[1,2-a]pyrimidin-9-yl)ethyl)amino)benzoic |
| | acid |
| 11-82 | 2-(((R)-1-(2-((S)-4-(2-cyanophenyl)-2-methylpiperazin-1-yl)-3,7- |
| | dimethyl-4-oxo-4H-pyrido[1,2-a]pyrimidin-9-yl)ethyl)amino)benzoic acid |
| 11-83 | 2-(((R)-1-(2-((R)-4-(2-cyanophenyl)-2-methylpiperazin-1-yl)-3,7- |
| | dimethyl-4-oxo-4H-pyrido[1,2-a]pyrimidin-9-yl)ethyl)amino)benzoic acid |
| 11-84 | 2-(((R)-1-(3-cyano-2-((R)-4-(3-cyanophenyl)-2-methylpiperazin-1-yl)- |
| | 7-methyl-4-oxo-4H-pyrido[1,2-a]pyrimidin-9-yl)ethyl)amino)benzoic acid |
| 11-85 | 2-(((R)-1-(3-cyano-2-((S)-4-(3-cyanophenyl)-2-methylpiperazin-1-yl)-7- |
| | methyl-4-oxo-4H-pyrido[1,2-a]pyrimidin-9-yl)ethyl)amino)benzoic acid |
| 11-86 | 2-(((R)-1-(2-((S)-4-(3-cyanophenyl)-2-methylpiperazin-1-yl)-3,7- |
| | dimethyl-4-oxo-4H-pyrido[1,2-a]pyrimidin-9-yl)ethyl)amino)benzoic acid |
| 11-87 | 2-(((R)-1-(2-((R)-4-(3-cyanophenyl)-2-methylpiperazin-1-yl)-3,7- |
| | dimethyl-4-oxo-4H-pyrido[1,2-a]pyrimidin-9-yl)ethyl)amino)benzoic acid |
| 11-88 | (R)-6-((1-(3-cyano-2-(4,4-difluoropiperidin-1-yl)-7-methyl-4-oxo-4H- |
| | pyrido[1,2-a]pyrimidin-9-yl)ethyl)amino)-2,3-difluorobenzoic acid |
| 11-89 | (R)-3-chloro-6-((1-(3-cyano-2-(4,4-difluoropiperidin-1-yl)-7-methyl-4- |
| | oxo-4H-pyrido[1,2-a]pyrimidin-9-yl)ethyl)amino)-2-fluorobenzoic acid |
| 11-90 | (R)-2-((1-(3-cyano-2-(4,4-difluoropiperidin-1-yl)-7-methyl-4-oxo-4H- |
| | pyrido[1,2-a]pyrimidin-9-yl)ethyl)amino)-5-fluorobenzoic acid |
| 11-91 | (R)-5-chloro-2-((1-(2-(4,4-difluoropiperidin-1-yl)-3,7-dimethyl-4-oxo- |
| | 4H-pyrido[1,2-a]pyrimidin-9-yl)ethyl)amino)benzoic acid |
| 11-92 | 2-(((R)-1-(2-((S)-4,4-difluoro-2-methylpiperidin-1-yl)-3,7-dimethyl-4- |
| | oxo-4H-pyrido[1,2-a]pyrimidin-9-yl)ethyl)amino)benzoic acid |
| 11-93 | 2-(((R)-1-(2-((R)-4,4-difluoro-2-methylpiperidin-1-yl)-3,7-dimethyl-4- |
| | oxo-4H-pyrido[1,2-a]pyrimidin-9-yl)ethyl)amino)benzoic acid |
| 11-94 | 2-(((R)-1-(2-((S)-2-(fluoromethyl)azetidin-1-yl)-3,7-dimethyl-4-oxo-4H- |
| | pyrido[1,2-a]pyrimidin-9-yl)ethyl)amino)benzoic acid; |
| 11-95 | 2-(((R)-1-(2-((R)-2-(fluoromethyl)azetidin-1-yl)-3,7-dimethyl-4-oxo-4H- |
| | pyrido[1,2-a]pyrimidin-9-yl)ethyl)amino)benzoic acid |
| 11-96 | 2-(((R)-1-(2-((R)-3,3-difluoro-2-methylazetidin-1-yl)-3,7-dimethyl-4- |
| | oxo-4H-pyrido[1,2-a]pyrimidin-9-yl)ethyl)amino)benzoic acid |
| 11-97 | 2-(((R)-1-(3,7-dimethyl-4-oxo-2-((S)-2-(trifluoromethyl)azetidin-1-yl)- |
| | 4H-pyrido[1,2-a]pyrimidin-9-yl)ethyl)amino)benzoic acid |
| 11-98 | 2-(((R)-1-(3,7-dimethyl-4-oxo-2-((R)-2-(trifluoromethyl)azetidin-1-yl)- |
| | 4H-pyrido[1,2-a]pyrimidin-9-yl)ethyl)amino)benzoic acid |
| 11-99 | (R)-2-((1-(2-(cyclobutylamino)-3,7-dimethyl-4-oxo-4H-pyrido[1,2- |
| | a]pyrimidin-9-yl)ethyl)amino)benzoic acid |
| 11-100 | 2-(((R)-1-(3,7-dimethyl-2-((R)-2-methylazetidin-1-yl)-4-oxo-4H- |
| | pyrido[1,2-a]pyrimidin-9-yl)ethyl)amino)benzoic acid |
| 11-101 | 2-(((R)-1-(3,7-dimethyl-2-((S)-2-methylazetidin-1-yl)-4-oxo-4H- |
| | pyrido[1,2-a]pyrimidin-9-yl)ethyl)amino)benzoic acid |
| 11-102 | (R)-2-((1-(3-cyano-2-(1,1-difluoro-5-azaspiro[2.3]hexan-5-yl)-7-methyl- |
| | 4-oxo-4H-pyrido[1,2-a]pyrimidin-9-yl)ethyl)amino)benzoic acid |
| 11-103 | (R)-2-((1-(2-(1,1-difluoro-5-azaspiro[2.3]hexan-5-yl)-3,7-dimethyl-4- |
| | oxo-4H-pyrido[1,2-a]pyrimidin-9-yl)ethyl)amino)benzoic acid |
| 11-104 | (R)-2-((1-(3-cyano-2-(3-(difluoromethyl)azetidin-1-yl)-7-methyl-4-oxo- |
| | 4H-pyrido[1,2-a]pyrimidin-9-yl)ethyl)amino)benzoic acid |
| 11-105 | (R)-2-((1-(2-(3-(difluoromethyl)azetidin-1-yl)-3,7-dimethyl-4-oxo-4H- |
| | pyrido[1,2-a]pyrimidin-9-yl)ethyl)amino)benzoic acid |
| 11-106 | (R)-2-((1-(3-cyano-7-methyl-4-oxo-2-(3-(trifluoromethyl)azetidin-1-yl)- |
| | 4H-pyrido[1,2-a]pyrimidin-9-yl)ethyl)amino)benzoic acid |
| 11-107 | (R)-2-((1-(3,7-dimethyl-4-oxo-2-(3-(trifluoromethyl)azetidin-1-yl)-4H- |
| | pyrido[1,2-a]pyrimidin-9-yl)ethyl)amino)benzoic acid |
| 11-108 | (R)-2-((1-(3-cyano-2-((2-cyanoallyl)amino)-7-methyl-4-oxo-4H- |
| | pyrido[1,2-a]pyrimidin-9-yl)ethyl)amino)benzoic acid |
| 11-109 | (R)-2-((1-(3-cyano-2-(3-cyanoazetidin-1-yl)-7-methyl-4-oxo-4H- |
| | pyrido[1,2-a]pyrimidin-9-yl)ethyl)amino)benzoic acid |
| 11-110 | (R)-2-((1-(2-((2-cyanoallyl)amino)-3,7-dimethyl-4-oxo-4H-pyrido[1,2- |
| | a]pyrimidin-9-yl)ethyl)amino)benzoic acid |
| 11-111 | (R)-2-((1-(3-cyano-2-(3-methoxyazetidin-1-yl)-7-methyl-4-oxo-4H- |
| | pyrido[1,2-a]pyrimidin-9-yl)ethyl)amino)benzoic acid |
| 11-112 | (R)-2-((1-(2-(3-methoxyazetidin-1-yl)-3,7-dimethyl-4-oxo-4H- |
| | pyrido[1,2-a]pyrimidin-9-yl)ethyl)amino)benzoic acid |
| 11-113 | (R)-2-((1-(3-cyano-2-(3-fluoroazetidin-1-yl)-7-methyl-4-oxo-4H- |
| | pyrido[1,2-a]pyrimidin-9-yl)ethyl)amino)benzoic acid |
| 11-114 | (R)-2-((1-(2-(3-fluoroazetidin-1-yl)-3,7-dimethyl-4-oxo-4H-pyrido[1,2- |
| | a]pyrimidin-9-yl)ethyl)amino)benzoic acid |
| 11-115 | (R)-2-((1-(2-(3,3-difluoroazetidin-1-yl)-3,7-dimethyl-4-oxo-4H- |
| | pyrido[1,2-a]pyrimidin-9-yl)ethyl)amino)benzoic acid |
| 11-116 | 2-(((R)-1-(3-cyano-2-((S)-3,3-difluoro-4-methoxypyrrolidin-1-yl)-7- |
| | methyl-4-oxo-4H-pyrido[1,2-a]pyrimidin-9-yl)ethyl)amino)benzoic acid |
| 11-117 | 2-(((R)-1-(2-((R)-3,3-difluoro-4-methoxypyrrolidin-1-yl)-3,7-dimethyl-4- |
| | oxo-4H-pyrido[1,2-a]pyrimidin-9-yl)ethyl)amino)benzoic acid |
| 11-118 | 2-(((R)-1-(2-((S)-3,3-difluoro-4-methoxypyrrolidin-1-yl)-3,7-dimethyl-4- |
| | oxo-4H-pyrido[1,2-a]pyrimidin-9-yl)ethyl)amino)benzoic acid |
| 11-119 | 2-(((R)-1-(2-((S)-3,3-difluoro-4-methylpyrrolidin-1-yl)-3,7-dimethyl-4- |
| | oxo-4H-pyrido[1,2-a]pyrimidin-9-yl)ethyl)amino)benzoic acid |
| 11-120 | 2-(((R)-1-(2-((R)-3,3-difluoro-4-methylpyrrolidin-1-yl)-3,7-dimethyl-4- |
| | oxo-4H-pyrido[1,2-a]pyrimidin-9-yl)ethyl)amino)benzoic acid |
| 11-121 | 2-(((1R)-1-(2-(3,3-difluoro-4-methylpyrrolidin-1-yl)-3,7-dimethyl-4-oxo- |
| | 4H-pyrido[1,2-a]pyrimidin-9-yl)ethyl)amino)benzoic acid |
| 11-122 | 2-(((R)-1-(2-((S)-3,3-difluoro-2-methylpyrrolidin-1-yl)-3,7-dimethyl-4- |
| | oxo-4H-pyrido[1,2-a]pyrimidin-9-yl)ethyl)amino)benzoic acid |
| 11-123 | 2-(((R)-1-(2-((R)-3,3-difluoro-2-methylpyrrolidin-1-yl)-3,7-dimethyl-4- |
| | oxo-4H-pyrido[1,2-a]pyrimidin-9-yl)ethyl)amino)benzoic acid |
| 11-124 | 2-(((R)-1-(3-cyano-2-((S)-4,4-difluoro-2-methylpyrrolidin-1-yl)-7- |
| | methyl-4-oxo-4H-pyrido[1,2-a]pyrimidin-9-yl)ethyl)amino)benzoic acid |
| 11-125 | 2-(((R)-1-(2-((R)-4,4-difluoro-2-methylpyrrolidin-1-yl)-3,7-dimethyl-4- |
| | oxo-4H-pyrido[1,2-a]pyrimidin-9-yl)ethyl)amino)benzoic acid |
| 11-126 | 2-(((R)-1-(2-((S)-4,4-difluoro-2-methylpyrrolidin-1-yl)-3,7-dimethyl-4- |
| | oxo-4H-pyrido[1,2-a]pyrimidin-9-yl)ethyl)amino)benzoic acid |
| 11-127 | (R)-2-((1-(2-(2-azabicyclo[2.1.1]hexan-2-yl)-3,7-dimethyl-4-oxo-4H- |
| | pyrido[1,2-a]pyrimidin-9-yl)ethyl)amino)benzoic acid |
| 11-128 | 6-chloro-3-(((R)-1-(3-cyano-2-((R)-4-(4-cyanophenyl)-2- |
| | methylpiperazin-1-yl)-7-methyl-4-oxo-4H-pyrido[1,2-a]pyrimidin-9- |
| | yl)ethyl)amino)picolinic acid |
| 11-129 | 6-chloro-3-(((R)-1-(3-cyano-2-((S)-4-(4-cyanophenyl)-2- |
| | methylpiperazin-1-yl)-7-methyl-4-oxo-4H-pyrido[1,2-a]pyrimidin-9- |
| | yl)ethyl)amino)picolinic acid |
| 11-130 | 6-chloro-3-(((R)-1-(3-cyano-2-((S)-4-(4-fluorophenyl)-2- |
| | methylpiperazin-1-yl)-7-methyl-4-oxo-4H-pyrido[1,2-a]pyrimidin-9- |
| | yl)ethyl)amino)picolinic acid |
| 11-131 | 6-chloro-3-(((R)-1-(3-cyano-2-((R)-4-(4-fluorophenyl)-2- |
| | methylpiperazin-1-yl)-7-methyl-4-oxo-4H-pyrido[1,2-a]pyrimidin-9- |
| | yl)ethyl)amino)picolinic acid |
| 11-132 | (R)-6-chloro-3-((1-(3-cyano-2-(4-(4-cyano-2-methylphenyl)piperazin-1- |
| | yl)-7-methyl-4-oxo-4H-pyrido[1,2-a]pyrimidin-9-yl)ethyl)amino)picolinic acid |
| 11-133 | (R)-6-chloro-3-((1-(2-(4-(4-cyano-2-methylphenyl)piperazin-1-yl)-3,7- |
| | dimethyl-4-oxo-4H-pyrido[1,2-a]pyrimidin-9-yl)ethyl)amino)picolinic acid |
| 11-134 | 2-(((1R)-1-(3-cyano-2-(6-(4-cyanophenyl)-3,6- |
| | diazabicyclo[3.1.1]heptan-3-yl)-7-methyl-4-oxo-4H-pyrido[1,2-a]pyrimidin-9- |
| | yl)ethyl)amino)benzoic acid |
| 11-135 | 2-(((1R)-1-(2-(6-(4-cyanophenyl)-3,6-diazabicyclo[3.1.1]heptan-3-yl)- |
| | 3,7-dimethyl-4-oxo-4H-pyrido[1,2-a]pyrimidin-9-yl)ethyl)amino)benzoic acid |
| 11-136 | (R)-2-((1-(3-cyano-7-methyl-4-oxo-2-(3,3,4,4-tetrafluoropyrrolidin-1- |
| | yl)-4H-pyrido[1,2-a]pyrimidin-9-yl)ethyl)amino)benzoic acid |
| 11-137 | (R)-2-((1-(3-cyano-2-(5,7-dihydro-6H-pyrrolo[3,4-b]pyrazin-6-yl)-7- |
| | methyl-4-oxo-4H-pyrido[1,2-a]pyrimidin-9-yl)ethyl)amino)benzoic acid |
| 11-138 | (R)-2-((1-(2-(5,7-dihydro-6H-pyrrolo[3,4-b]pyrazin-6-yl)-3,7-dimethyl-4- |
| | oxo-4H-pyrido[1,2-a]pyrimidin-9-yl)ethyl)amino)benzoic acid |
| 11-139 | (R)-6-chloro-3-((1-(2-(5,7-dihydro-6H-pyrrolo[3,4-b]pyrazin-6-yl)-3,7- |
| | dimethyl-4-oxo-4H-pyrido[1,2-a]pyrimidin-9-yl)ethyl)amino)picolinic acid |
| 11-140 | (R)-2-((1-(2-(3,3-difluoropyrrolidin-1-yl)-3,7-dimethyl-4-oxo-4H- |
| | pyrido[1,2-a]pyrimidin-9-yl)ethyl)amino)benzoic acid |
| 11-141 | (R)-2-((1-(2-(4,4-difluorocyclohexyl)-3,7-dimethyl-4-oxo-4H-pyrido[1,2- |
| | a]pyrimidin-9-yl)ethyl)amino)benzoic acid |
| 11-142 | (R)-4-((1-(3-cyano-2-(4,4-difluoropiperidin-1-yl)-7-methyl-4-oxo-4H- |
| | pyrido[1,2-a]pyrimidin-9-yl)ethyl)amino)nicotinic acid |
| 11-143 | 6-chloro-3-(((1R)-1-(3-cyano-2-(6,6-difluoro-3-azabicyclo[3.1.0]hexan- |
| | 3-yl)-7-methyl-4-oxo-4H-pyrido[1,2-a]pyrimidin-9-yl)ethyl)amino)picolinic |
| | acid |
| 11-144 | (R)-6-chloro-3-((1-(3-cyano-2-(5,6-difluoroisoindolin-2-yl)-7-methyl-4- |
| | oxo-4H-pyrido[1,2-a]pyrimidin-9-yl)ethyl)amino)picolinic acid |
| 11-145 | (R)-2-(4,4-difluoropiperidin-1-yl)-7-methyl-4-oxo-9-(1-(pyridazin-4- |
| | ylamino)ethyl)-4H-pyrido[1,2-a]pyrimidine-3-carbonitrile |
| 11-146 | (R)-4-((1-(3-cyano-2-(4,4-difluoropiperidin-1-yl)-7-methyl-4-oxo-4H- |
| | pyrido[1,2-a]pyrimidin-9-yl)ethyl)amino)pyridazine-3-carboxylic acid |
| 11-147 | (R)-6-chloro-3-((1-(3-cyano-2-(1,3-dihydro-2H-pyrrolo[3,4-c]pyridin-2- |
| | yl)-7-methyl-4-oxo-4H-pyrido[1,2-a]pyrimidin-9-yl)ethyl)amino)picolinic acid |
| 11-148 | (R)-2-((1-(3,7-dimethyl-2-(1-methyl-1,2,3,6-tetrahydropyridin-4-yl)-4- |
| | oxo-4H-pyrido[1,2-a]pyrimidin-9-yl)ethyl)amino)benzoic acid |
| 11-149 | (R)-2-((1-(3,7-dimethyl-2-(1-methylpiperidin-4-yl)-4-oxo-4H-pyrido[1,2- |
| | a]pyrimidin-9-yl)ethyl)amino)benzoic acid |
| 11-150 | (R)-2-((1-(3-cyano-2-(4-cyclopropylpiperazin-1-yl)-7-methyl-4-oxo-4H- |
| | pyrido[1,2-a]pyrimidin-9-yl)ethyl)amino)benzoic acid |
| 11-151 | (R)-2-((1-(2-(4-cyclopropylpiperazin-1-yl)-3,7-dimethyl-4-oxo-4H- |
| | pyrido[1,2-a]pyrimidin-9-yl)ethyl)amino)benzoic acid |
| 11-152 | (R)-2-((1-(3-cyano-7-methyl-2-(4-(oxetan-3-yl)piperazin-1-yl)-4-oxo- |
| | 4H-pyrido[1,2-a]pyrimidin-9-yl)ethyl)amino)benzoic acid |
| 11-153 | (R)-2-((1-(3,7-dimethyl-2-(4-(oxetan-3-yl)piperazin-1-yl)-4-oxo-4H- |
| | pyrido[1,2-a]pyrimidin-9-yl)ethyl)amino)benzoic acid |
| 11-154 | (R)-2-((1-(3-cyano-7-methyl-4-oxo-2-(4-(2,2,2-trifluoroethyl)piperazin- |
| | 1-yl)-4H-pyrido[1,2-a]pyrimidin-9-yl)ethyl)amino)benzoic acid |
| 11-155 | (R)-2-((1-(3-cyano-2-(((1-fluorocyclopropyl)methyl)amino)-7-methyl-4- |
| | oxo-4H-pyrido[1,2-a]pyrimidin-9-yl)ethyl)amino)benzoic acid |
| 11-156 | 2-(((R)-1-(3-cyano-2-((((R)-2,2-difluorocyclopropyl)methyl)amino)-7- |
| | methyl-4-oxo-4H-pyrido[1,2-a]pyrimidin-9-yl)ethyl)amino)benzoic acid |
| 11-157 | 2-(((R)-1-(3-cyano-2-((((S)-2,2-difluorocyclopropyl)methyl)amino)-7- |
| | methyl-4-oxo-4H-pyrido[1,2-a]pyrimidin-9-yl)ethyl)amino)benzoic acid |
| 11-158 | 2-(((1R)-1-(3-cyano-2-(((2,2-difluorocyclopropyl)methyl)amino)-7- |
| | methyl-4-oxo-4H-pyrido[1,2-a]pyrimidin-9-yl)ethyl)amino)benzoic acid |
| 11-159 | (R)-2-((1-(3-cyano-7-methyl-4-oxo-2-(((1- |
| | (trifluoromethyl)cyclopropyl)methyl)amino)-4H-pyrido[1,2-a]pyrimidin-9- |
| | yl)ethyl)amino)benzoic acid |
| 11-160 | 2-(((R)-1-(3-cyano-2-(((R)-1-cyclopropyl-2,2,2-trifluoroethyl)amino)-7- |
| | methyl-4-oxo-4H-pyrido[1,2-a]pyrimidin-9-yl)ethyl)amino)benzoic acid |
| 11-161 | 2-(((R)-1-(3-cyano-2-(((S)-1-cyclopropyl-2,2,2-trifluoroethyl)amino)-7- |
| | methyl-4-oxo-4H-pyrido[1,2-a]pyrimidin-9-yl)ethyl)amino)benzoic acid |
| 11-162 | 2-(((R)-1-(2-(((R)-1-cyclopropyl-2,2,2-trifluoroethyl)amino)-3,7- |
| | dimethyl-4-oxo-4H-pyrido[1,2-a]pyrimidin-9-yl)ethyl)amino)benzoic acid |
| 11-163 | 2-(((R)-1-(2-(((S)-1-cyclopropyl-2,2,2-trifluoroethyl)amino)-3,7- |
| | dimethyl-4-oxo-4H-pyrido[1,2-a]pyrimidin-9-yl)ethyl)amino)benzoic acid |
| 11-164 | 2-(((R)-1-(3-cyano-2-(((S)-1-cyclopropylethyl)amino)-7-methyl-4-oxo- |
| | 4H-pyrido[1,2-a]pyrimidin-9-yl)ethyl)amino)benzoic acid |
| 11-165 | 2-(((R)-1-(3-cyano-2-(((R)-1-cyclopropylethyl)amino)-7-methyl-4-oxo- |
| | 4H-pyrido[1,2-a]pyrimidin-9-yl)ethyl)amino)benzoic acid |
| 11-166 | 2-(((R)-1-(2-(((S)-1-cyclopropylethyl)amino)-3,7-dimethyl-4-oxo-4H- |
| | pyrido[1,2-a]pyrimidin-9-yl)ethyl)amino)benzoic acid |
| 11-167 | 2-(((R)-1-(2-(((R)-1-cyclopropylethyl)amino)-3,7-dimethyl-4-oxo-4H- |
| | pyrido[1,2-a]pyrimidin-9-yl)ethyl)amino)benzoic acid |
| 11-168 | (R)-2-((1-(3-cyano-7-(difluoromethyl)-2-(4,4-difluoropiperidin-1-yl)-4- |
| | oxo-4H-pyrido[1,2-a]pyrimidin-9-yl)ethyl)amino)benzoic acid |
| 11-169 | (S)-2-((1-(3-cyano-7-(difluoromethyl)-2-(4,4-difluoropiperidin-1-yl)-4- |
| | oxo-4H-pyrido[1,2-a]pyrimidin-9-yl)ethyl)amino)benzoic acid |
| 11-170 | 2-((1-(3-cyano-7-(difluoromethyl)-2-(4,4-difluoropiperidin-1-yl)-4-oxo- |
| | 4H-pyrido[1,2-a]pyrimidin-9-yl)ethyl)amino)benzoic acid |
| 11-171 | (R)-2-((1-(7-(difluoromethyl)-2-(4,4-difluoropiperidin-1-yl)-3-methyl-4- |
| | oxo-4H-pyrido[1,2-a]pyrimidin-9-yl)ethyl)amino)benzoic acid |
| 11-172 | (R)-2-((1-(3-cyano-2-(7-(4-cyanophenyl)-2,7-diazaspiro[3.5]nonan-2- |
| | yl)-7-methyl-4-oxo-4H-pyrido[1,2-a]pyrimidin-9-yl)ethyl)amino)benzoic acid |
| 11-173 | (R)-2-((1-(3-cyano-7-methyl-2-(4-(1-methyl-3-(trifluoromethyl)-1H- |
| | pyrazol-4-yl)piperazin-1-yl)-4-oxo-4H-pyrido[1,2-a]pyrimidin-9- |
| | yl)ethyl)amino)benzoic acid |
| 11-174 | 2-(((R)-1-(3-cyano-2-((1R,4R)-5-(4-cyanophenyl)-2,5- |
| | diazabicyclo[2.2.1]heptan-2-yl)-7-methyl-4-oxo-4H-pyrido[1,2-a]pyrimidin-9- |
| | yl)ethyl)amino)benzoic acid |
| 11-175 | 2-(((R)-1-(3-cyano-2-((1S,4S)-5-(4-cyanophenyl)-2,5- |
| | diazabicyclo[2.2.1]heptan-2-yl)-7-methyl-4-oxo-4H-pyrido[1,2-a]pyrimidin-9- |
| | yl)ethyl)amino)benzoic acid |
| 11-176 | 2-(((R)-1-(2-((1R,4R)-5-(4-cyanophenyl)-2,5- |
| | diazabicyclo[2.2.1]heptan-2-yl)-3,7-dimethyl-4-oxo-4H-pyrido[1,2- |
| | a]pyrimidin-9-yl)ethyl)amino)benzoic acid |
| 11-177 | 2-(((R)-1-(2-((1S,4S)-5-(4-cyanophenyl)-2,5- |
| | diazabicyclo[2.2.1]heptan-2-yl)-3,7-dimethyl-4-oxo-4H-pyrido[1,2- |
| | a]pyrimidin-9-yl)ethyl)amino)benzoic acid |
| 11-178 | 2-(((1R)-1-(2-(3,6-diazabicyclo[3.1.1]heptan-6-yl)-3-cyano-7-methyl-4- |
| | oxo-4H-pyrido[1,2-a]pyrimidin-9-yl)ethyl)amino)benzoic acid |
| 11-179 | 2-(((1R)-1-(3-cyano-2-(3-(4-cyanophenyl)-3,6- |
| | diazabicyclo[3.1.1]heptan-6-yl)-7-methyl-4-oxo-4H-pyrido[1,2-a]pyrimidin-9- |
| | yl)ethyl)amino)benzoic acid |
| 11-180 | 2-(((1R)-1-(2-(3,6-diazabicyclo[3.1.1]heptan-6-yl)-3,7-dimethyl-4-oxo- |
| | 4H-pyrido[1,2-a]pyrimidin-9-yl)ethyl)amino)benzoic acid |
| 11-181 | 2-(((1R)-1-(2-(3-(4-cyanophenyl)-3,6-diazabicyclo[3.1.1]heptan-6-yl)- |
| | 3,7-dimethyl-4-oxo-4H-pyrido[1,2-a]pyrimidin-9-yl)ethyl)amino)benzoic acid |
| 11-182 | 2-(((1R)-1-(3-cyano-2-(3-(4-cyanophenyl)-3,8- |
| | diazabicyclo[3.2.1]octan-8-yl)-7-methyl-4-oxo-4H-pyrido[1,2-a]pyrimidin-9- |
| | yl)ethyl)amino)benzoic acid |
| 11-183 | 2-(((1R)-1-(2-(3-(4-cyanophenyl)-3,8-diazabicyclo[3.2.1]octan-8-yl)- |
| | 3,7-dimethyl-4-oxo-4H-pyrido[1,2-a]pyrimidin-9-yl)ethyl)amino)benzoic acid |
| 11-184 | (R)-2-((1-(3-cyano-2-(4-(3-cyano-1-methyl-1H-pyrazol-4-yl)piperazin- |
| | 1-yl)-7-methyl-4-oxo-4H-pyrido[1,2-a]pyrimidin-9-yl)ethyl)amino)benzoic acid |
| 11-185 | (R)-2-((1-(2-(4-(3-cyano-1-methyl-1H-pyrazol-4-yl)piperazin-1-yl)-3,7- |
| | dimethyl-4-oxo-4H-pyrido[1,2-a]pyrimidin-9-yl)ethyl)amino)benzoic acid |
| 11-186 | (R)-2-((1-(3-cyano-7-methyl-4-oxo-2-(4-(tetrahydro-2H-pyran-4- |
| | yl)piperazin-1-yl)-4H-pyrido[1,2-a]pyrimidin-9-yl)ethyl)amino)benzoic acid |
| 11-187 | (R)-2-((1-(3,7-dimethyl-4-oxo-2-(4-(tetrahydro-2H-pyran-4- |
| | yl)piperazin-1-yl)-4H-pyrido[1,2-a]pyrimidin-9-yl)ethyl)amino)benzoic acid |
| 11-188 | (R)-2-((1-(2-(5,6-difluoroisoindolin-2-yl)-3,7-dimethyl-4-oxo-4H- |
| | pyrido[1,2-a]pyrimidin-9-yl)ethyl)amino)benzoic acid |
| 11-189 | 2-(((1R)-1-(2-(3,3-difluoro-8-azabicyclo[3.2.1]octan-8-yl)-3,7-dimethyl- |
| | 4-oxo-4H-pyrido[1,2-a]pyrimidin-9-yl)ethyl)amino)benzoic acid |
| 11-190 | (R)-2-((1-(3-cyano-2-(6-(4-cyanophenyl)-2,6-diazaspiro[3.3]heptan-2- |
| | yl)-7-methyl-4-oxo-4H-pyrido[1,2-a]pyrimidin-9-yl)ethyl)amino)benzoic acid |
| 11-191 | (R)-2-((1-(2-(6-(4-cyanophenyl)-2,6-diazaspiro[3.3]heptan-2-yl)-3,7- |
| | dimethyl-4-oxo-4H-pyrido[1,2-a]pyrimidin-9-yl)ethyl)amino)benzoic acid |
| 11-192 | 2-(((R)-1-(3-cyano-7-methyl-4-oxo-2-((((S)-tetrahydrofuran-3- |
| | yl)methyl)amino)-4H-pyrido[1,2-a]pyrimidin-9-yl)ethyl)amino)benzoic acid |
| 11-193 | 2-(((R)-1-(3-cyano-7-methyl-4-oxo-2-((((R)-tetrahydrofuran-3- |
| | yl)methyl)amino)-4H-pyrido[1,2-a]pyrimidin-9-yl)ethyl)amino)benzoic acid |
| 11-194 | 2-(((1R)-1-(3-cyano-7-methyl-4-oxo-2-(((tetrahydrofuran-3- |
| | yl)methyl)amino)-4H-pyrido[1,2-a]pyrimidin-9-yl)ethyl)amino)benzoic acid |
| 11-195 | 2-(((R)-1-(3,7-dimethyl-4-oxo-2-((((S)-tetrahydrofuran-3- |
| | yl)methyl)amino)-4H-pyrido[1,2-a]pyrimidin-9-yl)ethyl)amino)benzoic acid |
| 11-196 | 2-(((R)-1-(3,7-dimethyl-4-oxo-2-((((R)-tetrahydrofuran-3- |
| | yl)methyl)amino)-4H-pyrido[1,2-a]pyrimidin-9-yl)ethyl)amino)benzoic acid |
| 11-197 | 2-(((1R)-1-(3,7-dimethyl-4-oxo-2-(((tetrahydrofuran-3- |
| | yl)methyl)amino)-4H-pyrido[1,2-a]pyrimidin-9-yl)ethyl)amino)benzoic acid |
| 11-198 | (R)-2-((1-(3-cyano-2-(((3,3-difluorocyclobutyl)methyl)amino)-7-methyl- |
| | 4-oxo-4H-pyrido[1,2-a]pyrimidin-9-yl)ethyl)amino)benzoic acid |
| 11-199 | (R)-2-((1-(2-(((3,3-difluorocyclobutyl)methyl)amino)-3,7-dimethyl-4- |
| | oxo-4H-pyrido[1,2-a]pyrimidin-9-yl)ethyl)amino)benzoic acid |
| 11-200 | (R)-2-((1-(3-cyano-2-(5,5-difluoro-2-azaspiro[3.3]heptan-2-yl)-7- |
| | methyl-4-oxo-4H-pyrido[1,2-a]pyrimidin-9-yl)ethyl)amino)benzoic acid |
| 11-201 | (R)-2-((1-(2-(5,5-difluoro-2-azaspiro[3.3]heptan-2-yl)-3,7-dimethyl-4- |
| | oxo-4H-pyrido[1,2-a]pyrimidin-9-yl)ethyl)amino)benzoic acid |
| 11-202 | (R)-2-((1-(2-(6,6-difluoro-2-azaspiro[3.3]heptan-2-yl)-3,7-dimethyl-4- |
| | oxo-4H-pyrido[1,2-a]pyrimidin-9-yl)ethyl)amino)benzoic acid |
| 11-203 | (R)-2-((1-(2-((cyclopropylmethyl)(methyl)amino)-3,7-dimethyl-4-oxo- |
| | 4H-pyrido[1,2-a]pyrimidin-9-yl)ethyl)amino)benzoic acid |
| 11-204 | (R)-2-((1-(3-cyano-7-methyl-2-((oxetan-3-ylmethyl)amino)-4-oxo-4H- |
| | pyrido[1,2-a]pyrimidin-9-yl)ethyl)amino)benzoic acid |
| 11-205 | (R)-2-((1-(3,7-dimethyl-2-((oxetan-3-ylmethyl)amino)-4-oxo-4H- |
| | pyrido[1,2-a]pyrimidin-9-yl)ethyl)amino)benzoic acid |
| 11-206 | (R)-6-chloro-3-((1-(3-cyano-2-((cyclopropylmethyl)amino)-7-methyl-4- |
| | oxo-4H-pyrido[1,2-a]pyrimidin-9-yl)ethyl)amino)picolinic acid |
| 11-207 | (R)-2-((1-(2-((cyclopropylmethyl)amino)-3,7-dimethyl-4-oxo-4H- |
| | pyrido[1,2-a]pyrimidin-9-yl)ethyl)amino)benzoic acid |
| 11-208 | (R)-2-((1-(3-cyano-7-methyl-4-oxo-2-(2-oxa-7-azaspiro[3.5]nonan-7- |
| | yl)-4H-pyrido[1,2-a]pyrimidin-9-yl)ethyl)amino)benzoic acid |
| 11-209 | 2-(((R)-1-(3-cyano-7-methyl-4-oxo-2-((R)-3-(trifluoromethyl)piperidin- |
| | 1-yl)-4H-pyrido[1,2-a]pyrimidin-9-yl)ethyl)amino)benzoic acid |
| 11-210 | 2-(((R)-1-(3-cyano-7-methyl-4-oxo-2-((S)-3-(trifluoromethyl)piperidin- |
| | 1-yl)-4H-pyrido[1,2-a]pyrimidin-9-yl)ethyl)amino)benzoic acid |
| 11-211 | 2-(((R)-1-(3,7-dimethyl-4-oxo-2-((R)-3-(trifluoromethyl)piperidin-1-yl)- |
| | 4H-pyrido[1,2-a]pyrimidin-9-yl)ethyl)amino)benzoic acid |
| 11-212 | 2-(((R)-1-(3,7-dimethyl-4-oxo-2-((S)-3-(trifluoromethyl)piperidin-1-yl)- |
| | 4H-pyrido[1,2-a]pyrimidin-9-yl)ethyl)amino)benzoic acid |
| 11-213 | (R)-2-((1-(3,7-dimethyl-4-oxo-2-(1,1,2,2-tetrafluoro-6- |
| | azaspiro[2.5]octan-6-yl)-4H-pyrido[1,2-a]pyrimidin-9-yl)ethyl)amino)benzoic |
| | acid |
| 11-214 | 2-(((1R)-1-(3-cyano-7-methyl-4-oxo-2-(1,2,3,4-tetrahydro-1,4- |
| | epiminonaphthalen-9-yl)-4H-pyrido[1,2-a]pyrimidin-9-yl)ethyl)amino)benzoic |
| | acid |
| 11-215 | 2-(((1R)-1-(3,7-dimethyl-4-oxo-2-(1,2,3,4-tetrahydro-1,4- |
| | epiminonaphthalen-9-yl)-4H-pyrido[1,2-a]pyrimidin-9-yl)ethyl)amino)benzoic |
| | acid |
| 11-216 | (R)-2-((1-(3-cyano-7-methyl-4-oxo-2-(3-(trifluoromethyl)-5,6-dihydro- |
| | [1,2,4]triazolo[4,3-a]pyrazin-7(8H)-yl)-4H-pyrido[1,2-a]pyrimidin-9- |
| | yl)ethyl)amino)benzoic acid |
| 11-217 | (R)-2-((1-(3,7-dimethyl-4-oxo-2-(3-(trifluoromethyl)-5,6-dihydro- |
| | [1,2,4]triazolo[4,3-a]pyrazin-7(8H)-yl)-4H-pyrido[1,2-a]pyrimidin-9- |
| | yl)ethyl)amino)benzoic acid |
| 11-218 | (R)-2-((1-(3-cyano-2-(1,1-difluoro-6-azaspiro[2.5]octan-6-yl)-7-methyl- |
| | 4-oxo-4H-pyrido[1,2-a]pyrimidin-9-yl)ethyl)amino)benzoic acid |
| 11-219 | (R)-2-((1-(2-(1,1-difluoro-6-azaspiro[2.5]octan-6-yl)-3,7-dimethyl-4- |
| | oxo-4H-pyrido[1,2-a]pyrimidin-9-yl)ethyl)amino)benzoic acid |
| 11-220 | (R)-2-((1-(3-cyano-7-methyl-4-oxo-2-(6-azaspiro[2.5]octan-6-yl)-4H- |
| | pyrido[1,2-a]pyrimidin-9-yl)ethyl)amino)benzoic acid |
| 11-221 | (R)-2-((1-(3,7-dimethyl-4-oxo-2-(6-azaspiro[2.5]octan-6-yl)-4H- |
| | pyrido[1,2-a]pyrimidin-9-yl)ethyl)amino)benzoic acid |
| 11-222 | 2-(((1R)-1-(2-(6,6-difluoro-3-azabicyclo[3.1.0]hexan-3-yl)-3,7-dimethyl- |
| | 4-oxo-4H-pyrido[1,2-a]pyrimidin-9-yl)ethyl)amino)benzoic acid |
| 11-223 | 2-(((1R)-1-(2-(3-azabicyclo[3.1.0]hexan-3-yl)-3-cyano-7-methyl-4-oxo- |
| | 4H-pyrido[1,2-a]pyrimidin-9-yl)ethyl)amino)benzoic acid |
| 11-224 | 2-(((1R)-1-(2-(3-azabicyclo[3.1.0]hexan-3-yl)-3,7-dimethyl-4-oxo-4H- |
| | pyrido[1,2-a]pyrimidin-9-yl)ethyl)amino)benzoic acid |
| 11-225 | (R)-2-((1-(3-cyano-2-(4-(3-cyanophenyl)piperazin-1-yl)-7-methyl-4- |
| | oxo-4H-pyrido[1,2-a]pyrimidin-9-yl)ethyl)amino)benzoic acid |
| 11-226 | (R)-2-((1-(2-(4-(3-cyanophenyl)piperazin-1-yl)-3,7-dimethyl-4-oxo-4H- |
| | pyrido[1,2-a]pyrimidin-9-yl)ethyl)amino)benzoic acid |
| 11-227 | (R)-2-((1-(2-(3-azabicyclo[3.1.1]heptan-3-yl)-3-cyano-7-methyl-4-oxo- |
| | 4H-pyrido[1,2-a]pyrimidin-9-yl)ethyl)amino)benzoic acid |
| 11-228 | 2-(((R)-1-(2-((1R,4S)-6,6-difluoro-2-azabicyclo[2.2.1]heptan-2-yl)-3,7- |
| | dimethyl-4-oxo-4H-pyrido[1,2-a]pyrimidin-9-yl)ethyl)amino)benzoic acid |
| 11-229 | 6-chloro-3-(((1R)-1-(2-(6,6-difluoro-2-azabicyclo[2.2.1]heptan-2-yl)- |
| | 3,7-dimethyl-4-oxo-4H-pyrido[1,2-a]pyrimidin-9-yl)ethyl)amino)picolinic acid |
| 11-230 | 2-(((R)-1-(3-cyano-2-((1R,4R)-5,5-difluoro-2-azabicyclo[2.2.1]heptan- |
| | 2-yl)-7-methyl-4-oxo-4H-pyrido[1,2-a]pyrimidin-9-yl)ethyl)amino)benzoic acid |
| 11-231 | 2-(((R)-1-(2-((1S,4S)-5,5-difluoro-2-azabicyclo[2.2.1]heptan-2-yl)-3,7- |
| | dimethyl-4-oxo-4H-pyrido[1,2-a]pyrimidin-9-yl)ethyl)amino)benzoic acid |
| 11-232 | (R)-5-ethynyl-2-((1-(7-methyl-4-oxo-2-(piperidin-1-yl)-4H-pyrido[1,2- |
| | a]pyrimidin-9-yl)ethyl)amino)benzoic acid |
| 11-233 | (R)-2-((1-(2-(1-(4-cyanophenyl)-1H-pyrazol-4-yl)-3,7-dimethyl-4-oxo- |
| | 4H-pyrido[1,2-a]pyrimidin-9-yl)ethyl)amino)benzoic acid |
| 11-234 | (R)-2-((1-(3,7-dimethyl-2-(1-methyl-1H-imidazol-4-yl)-4-oxo-4H- |
| | pyrido[1,2-a]pyrimidin-9-yl)ethyl)amino)benzoic acid |
| 11-235 | (R)-2-((1-(2-(1H-imidazol-4-yl)-3,7-dimethyl-4-oxo-4H-pyrido[1,2- |
| | a]pyrimidin-9-yl)ethyl)amino)benzoic acid |
| 11-236 | (R)-2-((1-(2-(1-(4-cyanophenyl)-1H-imidazol-4-yl)-3,7-dimethyl-4-oxo- |
| | 4H-pyrido[1,2-a]pyrimidin-9-yl)ethyl)amino)benzoic acid |
| 11-237 | (R)-2-((1-(2-(1-(4-cyanophenyl)-1H-imidazol-4-yl)-7-methyl-4-oxo-4H- |
| | pyrido[1,2-a]pyrimidin-9-yl)ethyl)amino)benzoic acid |
| 11-238 | (R)-2-((1-(2-(3-(2-cyanopropan-2-yl)phenyl)-3,7-dimethyl-4-oxo-4H- |
| | pyrido[1,2-a]pyrimidin-9-yl)ethyl)amino)benzoic acid |
| 11-239 | (R)-2-((1-(2-(3-(2-cyanopropan-2-yl)phenyl)-7-methyl-4-oxo-4H- |
| | pyrido[1,2-a]pyrimidin-9-yl)ethyl)amino)benzoic acid |
| 11-240 | (R)-2-((1-(2-(1-(4-cyanophenyl)-1H-pyrazol-4-yl)-7-methyl-4-oxo-4H- |
| | pyrido[1,2-a]pyrimidin-9-yl)ethyl)amino)benzoic acid |
| 11-241 | 2-((1-(2-(4,4-difluoropiperidin-1-yl)-3,7-dimethyl-4-oxo-4H-pyrido[1,2- |
| | a]pyrimidin-9-yl)prop-2-yn-1-yl)amino)benzoic acid |
| 11-242 | (R)-2-((1-(2-chloro-3,7-dimethyl-4-oxo-4H-pyrido[1,2-a]pyrimidin-9- |
| | yl)ethyl)amino)benzoic acid |
| 11-243 | (R)-2-((1-(2-chloro-3-cyano-7-methyl-4-oxo-4H-pyrido[1,2-a]pyrimidin- |
| | 9-yl)ethyl)amino)benzoic acid |
| 11-244 | (R)-2-((1-(3-cyano-2-cyclopropyl-7-methyl-4-oxo-4H-pyrido[1,2- |
| | a]pyrimidin-9-yl)ethyl)amino)benzoic acid |
| 11-245 | 2-(((R)-1-(3,7-dimethyl-4-oxo-2-(((R)-tetrahydrofuran-3-yl)oxy)-4H- |
| | pyrido[1,2-a]pyrimidin-9-yl)ethyl)amino)benzoic acid |
| 11-246 | 2-(((R)-1-(3,7-dimethyl-4-oxo-2-(((S)-tetrahydrofuran-3-yl)oxy)-4H- |
| | pyrido[1,2-a]pyrimidin-9-yl)ethyl)amino)benzoic acid |
| 11-247 | 2-(((R)-1-(3-cyano-7-methyl-4-oxo-2-(((R)-tetrahydrofuran-3-yl)oxy)- |
| | 4H-pyrido[1,2-a]pyrimidin-9-yl)ethyl)amino)benzoic acid |
| 11-248 | 2-(((R)-1-(3-cyano-7-methyl-4-oxo-2-(((S)-tetrahydrofuran-3-yl)oxy)- |
| | 4H-pyrido[1,2-a]pyrimidin-9-yl)ethyl)amino)benzoic acid |
| 11-249 | (R)-2-((1-(3,7-dimethyl-2-(4-(4-(methylcarbamoyl)phenyl)piperazin-1- |
| | yl)-4-oxo-4H-pyrido[1,2-a]pyrimidin-9-yl)ethyl)amino)benzoic acid |
| 11-250 | (R)-2-((1-(2-(4-(4-carbamoylphenyl)piperazin-1-yl)-3,7-dimethyl-4-oxo- |
| | 4H-pyrido[1,2-a]pyrimidin-9-yl)ethyl)amino)benzoic acid |
| 11-251 | (R)-2-((1-(2-(4-(3-cyano-1-methyl-1H-indazol-7-yl)piperazin-1-yl)-3,7- |
| | dimethyl-4-oxo-4H-pyrido[1,2-a]pyrimidin-9-yl)ethyl)amino)benzoic acid |
| 11-252 | (R)-2-((1-(2-(4-(1H-pyrazol-3-yl)piperidin-1-yl)-3,7-dimethyl-4-oxo-4H- |
| | pyrido[1,2-a]pyrimidin-9-yl)ethyl)amino)benzoic acid |
| 11-253 | (R)-2-((1-(2-((4-cyanophenyl)ethynyl)-3,7-dimethyl-4-oxo-4H- |
| | pyrido[1,2-a]pyrimidin-9-yl)ethyl)amino)benzoic acid |
| 11-254 | (R)-2-((1-(2-(4-(4-cyano-6-hydroxynaphthalen-1-yl)piperazin-1-yl)-3,7- |
| | dimethyl-4-oxo-4H-pyrido[1,2-a]pyrimidin-9-yl)ethyl)amino)benzoic acid |
| 11-255 | (R)-2-((1-(2-(4-(4-cyano-7-hydroxynaphthalen-1-yl)piperazin-1-yl)-3,7- |
| | dimethyl-4-oxo-4H-pyrido[1,2-a]pyrimidin-9-yl)ethyl)amino)benzoic acid |
| 11-256 | (R)-2-((1-(2-(1-acetylpiperidin-4-yl)-3-cyano-7-methyl-4-oxo-4H- |
| | pyrido[1,2-a]pyrimidin-9-yl)ethyl)amino)benzoic acid |
| 11-257 | 2-((1-(2-(4-(4-(aminomethyl)phenyl)piperazin-1-yl)-7-methyl-4-oxo-4H- |
| | pyrido[1,2-a]pyrimidin-9-yl)ethyl)amino)benzoic acid |
| 11-258 | (R)-2-((1-(2-(4-(5-cyanopyrimidin-2-yl)piperazin-1-yl)-3,7-dimethyl-4- |
| | oxo-4H-pyrido[1,2-a]pyrimidin-9-yl)ethyl)amino)benzoic acid |
| 11-259 | 2-(((1R)-1-(3,7-dimethyl-4-oxo-2-(tetrahydro-1H-thieno[3,4-c]pyrrol- |
| | 5(3H)-yl)-4H-pyrido[1,2-a]pyrimidin-9-yl)ethyl)amino)benzoic acid |
| 11-260 | (R)-2-((1-(3-cyano-2-(4-(2-cyanophenyl)piperazin-1-yl)-7-methyl-4- |
| | oxo-4H-pyrido[1,2-a]pyrimidin-9-yl)ethyl)amino)benzoic acid |
| 11-261 | (R)-2-((1-(3-cyano-7-methyl-2-(4-(1-methyl-1H-pyrazole-4- |
| | carbonyl)piperazin-1-yl)-4-oxo-4H-pyrido[1,2-a]pyrimidin-9- |
| | yl)ethyl)amino)benzoic acid |
| 11-262 | (R)-2-((1-(3,7-dimethyl-2-(4-(1-methyl-1H-pyrazole-4- |
| | carbonyl)piperazin-1-yl)-4-oxo-4H-pyrido[1,2-a]pyrimidin-9- |
| | yl)ethyl)amino)benzoic acid |
| 11-263 | (R)-2-((1-(2-(4-benzoylpiperazin-1-yl)-3-cyano-7-methyl-4-oxo-4H- |
| | pyrido[1,2-a]pyrimidin-9-yl)ethyl)amino)benzoic acid |
| 11-264 | (R)-2-((1-(3-cyano-7-methyl-2-(4-(1-methyl-1H-pyrazol-4-yl)piperazin- |
| | 1-yl)-4-oxo-4H-pyrido[1,2-a]pyrimidin-9-yl)ethyl)amino)benzoic acid |
| 11-265 | (R)-2-((1-(3,7-dimethyl-2-(4-(1-methyl-1H-pyrazol-4-yl)piperazin-1-yl)- |
| | 4-oxo-4H-pyrido[1,2-a]pyrimidin-9-yl)ethyl)amino)benzoic acid |
| 11-266 | (R)-2-((1-(3-cyano-7-methyl-4-oxo-2-(4-(o-tolyl)piperazin-1-yl)-4H- |
| | pyrido[1,2-a]pyrimidin-9-yl)ethyl)amino)benzoic acid |
| 11-267 | (R)-2-((1-(3,7-dimethyl-4-oxo-2-(4-(o-tolyl)piperazin-1-yl)-4H- |
| | pyrido[1,2-a]pyrimidin-9-yl)ethyl)amino)benzoic acid |
| 11-268 | 2-(((1R)-1-(3,7-dimethyl-2-(2-methyl-4-phenylpiperazin-1-yl)-4-oxo- |
| | 4H-pyrido[1,2-a]pyrimidin-9-yl)ethyl)amino)benzoic acid |
| 11-269 | 2-(((1R)-1-(2-(4-(4-cyanophenyl)-2-methylpiperazin-1-yl)-3,7-dimethyl- |
| | 4-oxo-4H-pyrido[1,2-a]pyrimidin-9-yl)ethyl)amino)benzoic acid |
| 11-270 | 2-(((R)-1-(3-cyano-2-((R)-4-(4-cyanophenyl)-3-methylpiperazin-1-yl)- |
| | 7-methyl-4-oxo-4H-pyrido[1,2-a]pyrimidin-9-yl)ethyl)amino)benzoic acid |
| 11-271 | 2-(((1R)-1-(3-cyano-2-(4-(4-cyanophenyl)-3-methylpiperazin-1-yl)-7- |
| | methyl-4-oxo-4H-pyrido[1,2-a]pyrimidin-9-yl)ethyl)amino)benzoic acid |
| 11-272 | (R)-2-((1-(3-cyano-2-(4-(4-cyanophenyl)piperazin-1-yl)-7-methyl-4- |
| | oxo-4H-pyrido[1,2-a]pyrimidin-9-yl)ethyl)amino)benzoic acid |
| 11-273 | (R)-2-((1-(3,7-dimethyl-4-oxo-2-(4-phenylpiperazin-1-yl)-4H- |
| | pyrido[1,2-a]pyrimidin-9-yl)ethyl)amino)benzoic acid |
| 11-274 | (R)-4-((1-(3-cyano-2-(isoindolin-2-yl)-7-methyl-4-oxo-4H-pyrido[1,2- |
| | a]pyrimidin-9-yl)ethyl)amino)nicotinic acid |
| 11-275 | (R)-4-((1-(2-(isoindolin-2-yl)-3,7-dimethyl-4-oxo-4H-pyrido[1,2- |
| | a]pyrimidin-9-yl)ethyl)amino)nicotinic acid |
| 11-276 | (R)-6-chloro-4-((1-(2-(isoindolin-2-yl)-3,7-dimethyl-4-oxo-4H- |
| | pyrido[1,2-a]pyrimidin-9-yl)ethyl)amino)pyridazine-3-carboxylic acid |
| 11-277 | (R)-4-((1-(2-(isoindolin-2-yl)-3,7-dimethyl-4-oxo-4H-pyrido[1,2- |
| | a]pyrimidin-9-yl)ethyl)amino)pyridazine-3-carboxylic acid |
| 11-278 | (R)-3,7-dimethyl-9-(1-((1-methyl-2-oxo-1,2-dihydropyridin-3- |
| | yl)amino)ethyl)-2-(piperidin-1-yl)-4H-pyrido[1,2-a]pyrimidin-4-one |
| 11-279 | (R)-6-chloro-3-((1-(2-(4,4-dimethylpiperidin-1-yl)-3,7-dimethyl-4-oxo- |
| | 4H-pyrido[1,2-a]pyrimidin-9-yl)ethyl)amino)picolinic acid |
| 11-280 | (R)-5-chloro-2-((1-(2-(isoindolin-2-yl)-3,7-dimethyl-4-oxo-4H- |
| | pyrido[1,2-a]pyrimidin-9-yl)ethyl)amino)benzoic acid |
| 11-281 | (R)-5-chloro-2-((1-(3,7-dimethyl-4-oxo-2-(piperidin-1-yl)-4H-pyrido[1,2- |
| | a]pyrimidin-9-yl)ethyl)amino)benzoic acid |
| 11-282 | (R)-2-((1-(3-cyano-2-((3-(hydroxymethyl)bicyclo[1.1.1]pentan-1- |
| | yl)amino)-7-methyl-4-oxo-4H-pyrido[1,2-a]pyrimidin-9- |
| | yl)ethyl)amino)benzoic acid |
| 11-283 | (R)-2-((1-(3-cyano-7-methyl-2-(4-(methylsulfonyl)piperazin-1-yl)-4- |
| | oxo-4H-pyrido[1,2-a]pyrimidin-9-yl)ethyl)amino)benzoic acid |
| 11-284 | (R)-2-((1-(3-cyano-7-methyl-4-oxo-2-(3-oxopiperazin-1-yl)-4H- |
| | pyrido[1,2-a]pyrimidin-9-yl)ethyl)amino)benzoic acid |
| 11-285 | 2-(((1R)-1-(3-cyano-7-methyl-4-oxo-2-(1-oxa-6-azaspiro[3.4]octan-6- |
| | yl)-4H-pyrido[1,2-a]pyrimidin-9-yl)ethyl)amino)benzoic acid |
| 11-286 | (R)-2-((1-(3-cyano-2-(1,3-dihydro-2H-pyrrolo[3,4-c]pyridin-2-yl)-7- |
| | methyl-4-oxo-4H-pyrido[1,2-a]pyrimidin-9-yl)ethyl)amino)benzoic acid |
| 11-287 | (R)-2-((1-(3-cyano-2-(5,7-dihydro-6H-pyrrolo[3,4-b]pyridin-6-yl)-7- |
| | methyl-4-oxo-4H-pyrido[1,2-a]pyrimidin-9-yl)ethyl)amino)benzoic acid |
| 11-288 | 2-(((1R)-1-(3-cyano-2-(1-(hydroxymethyl)isoindolin-2-yl)-7-methyl-4- |
| | oxo-4H-pyrido[1,2-a]pyrimidin-9-yl)ethyl)amino)benzoic acid |
| 11-289 | 2-(((1R)-1-(3-cyano-7-methyl-4-oxo-2-(2-oxo-1,7- |
| | diazaspiro[4.4]nonan-7-yl)-4H-pyrido[1,2-a]pyrimidin-9- |
| | yl)ethyl)amino)benzoic acid |
| 11-290 | (R)-2-((1-(7-fluoro-2-(isoindolin-2-yl)-3-methyl-4-oxo-4H-pyrido[1,2- |
| | a]pyrimidin-9-yl)ethyl)amino)benzoic acid |
| 11-291 | (R)-2-((1-(7-chloro-2-(isoindolin-2-yl)-3-methyl-4-oxo-4H-pyrido[1,2- |
| | a]pyrimidin-9-yl)ethyl)amino)benzoic acid |
| 11-292 | (R)-5-((1-(3-cyano-2-(4,4-difluoropiperidin-1-yl)-7-methyl-4-oxo-4H- |
| | pyrido[1,2-a]pyrimidin-9-yl)ethyl)amino)thiazole-4-carboxylic acid |
| 11-293 | (R)-2-((1-(3-cyano-7-methyl-4-oxo-2-(piperidin-1-yl)-4H-pyrido[1,2- |
| | a]pyrimidin-9-yl)ethyl)amino)-5-fluorobenzoic acid |
| 11-294 | 2-(((R)-1-(2-((1R,4S)-2-azabicyclo[2.2.1]heptan-2-yl)-3-cyano-7- |
| | methyl-4-oxo-4H-pyrido[1,2-a]pyrimidin-9-yl)ethyl)amino)benzoic acid |
| 11-295 | (R)-2-((1-(3-cyano-7-methyl-2-(4-methylpiperazin-1-yl)-4-oxo-4H- |
| | pyrido[1,2-a]pyrimidin-9-yl)ethyl)amino)benzoic acid |
| 11-296 | (R)-2-((1-(3-cyano-7-methyl-4-oxo-2-(piperazin-1-yl)-4H-pyrido[1,2- |
| | a]pyrimidin-9-yl)ethyl)amino)benzoic acid |
| 11-297 | (R)-2-((1-(3-cyano-2-(3,3-difluoropiperidin-1-yl)-7-methyl-4-oxo-4H- |
| | pyrido[1,2-a]pyrimidin-9-yl)ethyl)amino)benzoic acid |
| 11-298 | (R)-6-chloro-3-((1-(2-(4,4-difluoropiperidin-1-yl)-3,7-dimethyl-4-oxo- |
| | 4H-pyrido[1,2-a]pyrimidin-9-yl)ethyl)amino)picolinic acid |
| 11-299 | 7-methyl-4-oxo-9-(1-(phenylamino)ethyl)-2-(piperidin-1-yl)-4H- |
| | pyrido[1,2-a]pyrimidine-3-carbonitrile |
| 11-300 | (S)-2-(isoindolin-2-yl)-7-methyl-4-oxo-9-(1-(phenylamino)ethyl)-4H- |
| | pyrido[1,2-a]pyrimidine-3-carbonitrile |
| 11-301 | (R)-2-(isoindolin-2-yl)-7-methyl-4-oxo-9-(1-(phenylamino)ethyl)-4H- |
| | pyrido[1,2-a]pyrimidine-3-carbonitrile |
| 11-302 | 2-(isoindolin-2-yl)-7-methyl-4-oxo-9-(1-(phenylamino)ethyl)-4H- |
| | pyrido[1,2-a]pyrimidine-3-carbonitrile |
| 11-303 | (R)-2-((1-(2-(4,4-difluoropiperidin-1-yl)-3,7-dimethyl-4-oxo-4H- |
| | pyrido[1,2-a]pyrimidin-9-yl)ethyl)amino)benzoic acid |
| 11-304 | (R)-2-((1-(2-(4,6-dihydro-5H-pyrrolo[3,4-d]thiazol-5-yl)-3,7-dimethyl-4- |
| | oxo-4H-pyrido[1,2-a]pyrimidin-9-yl)ethyl)amino)benzoic acid |
| 11-305 | (R)-2-((1-(3-cyano-7-methyl-2-(2-methyl-6,7-dihydrothiazolo[4,5- |
| | c]pyridin-5(4H)-yl)-4-oxo-4H-pyrido[1,2-a]pyrimidin-9-yl)ethyl)amino)benzoic |
| | acid |
| 11-306 | (R)-2-((1-(3-cyano-2-(6,7-dihydrothiazolo[4,5-c]pyridin-5(4H)-yl)-7- |
| | methyl-4-oxo-4H-pyrido[1,2-a]pyrimidin-9-yl)ethyl)amino)benzoic acid |
| 11-307 | (R)-2-((1-(3-cyano-2-(isoindolin-2-yl)-7-methyl-4-oxo-4H-pyrido[1,2- |
| | a]pyrimidin-9-yl)ethyl)amino)-6-fluorobenzoic acid |
| 11-308 | (R)-2-((1-(3-cyano-2-(isobutylamino)-7-methyl-4-oxo-4H-pyrido[1,2- |
| | a]pyrimidin-9-yl)ethyl)amino)benzoic acid |
| 11-309 | (R)-2-((1-(2-(isobutylamino)-3,7-dimethyl-4-oxo-4H-pyrido[1,2- |
| | a]pyrimidin-9-yl)ethyl)amino)benzoic acid |
| 11-310 | (R)-5-cyano-2-((1-(2-(isoindolin-2-yl)-3,7-dimethyl-4-oxo-4H- |
| | pyrido[1,2-a]pyrimidin-9-yl)ethyl)amino)benzoic acid |
| 11-311 | (R)-2-((1-(2-(4-(dimethylamino)piperidin-1-yl)-3,7-dimethyl-4-oxo-4H- |
| | pyrido[1,2-a]pyrimidin-9-yl)ethyl)amino)benzoic acid |
| 11-312 | (R)-2-((1-(3-cyano-2-(4-hydroxypiperidin-1-yl)-7-methyl-4-oxo-4H- |
| | pyrido[1,2-a]pyrimidin-9-yl)ethyl)amino)benzoic acid |
| 11-313 | (R)-2-((1-(3-cyano-2-(4-(dimethylamino)piperidin-1-yl)-7-methyl-4-oxo- |
| | 4H-pyrido[1,2-a]pyrimidin-9-yl)ethyl)amino)benzoic acid |
| 11-314 | (R)-2-((1-(3-cyano-7-methyl-2-(4-methylpiperidin-1-yl)-4-oxo-4H- |
| | pyrido[1,2-a]pyrimidin-9-yl)ethyl)amino)benzoic acid |
| 11-315 | 2-(3-cyano-7-methyl-4-oxo-2-(piperidin-1-yl)-4H-pyrido[1,2- |
| | a]pyrimidine-9-carboxamido)cyclohexane-1-carboxylic acid |
| 11-316 | 2-(1-(3-cyano-7-methyl-4-oxo-2-(piperidin-1-yl)-4H-pyrido[1,2- |
| | a]pyrimidine-9-carboxamido)cyclobutyl)acetic acid |
| 11-317 | (R)-1-((1-(3-cyano-7-methyl-4-oxo-2-(piperidin-1-yl)-4H-pyrido[1,2- |
| | a]pyrimidin-9-yl)ethyl)carbamoyl)cyclobutane-1-carboxylic acid |
| 11-318 | (R)-2-((1-(3-cyano-7-methyl-2-morpholino-4-oxo-4H-pyrido[1,2- |
| | a]pyrimidin-9-yl)ethyl)amino)benzoic acid |
| 11-319 | (R)-2-((1-(2-(azepan-1-yl)-3-cyano-7-methyl-4-oxo-4H-pyrido[1,2- |
| | a]pyrimidin-9-yl)ethyl)amino)benzoic acid |
| 11-320 | (R)-2-((1-(3-cyano-2-(diethylamino)-7-methyl-4-oxo-4H-pyrido[1,2- |
| | a]pyrimidin-9-yl)ethyl)amino)benzoic acid |
| 11-321 | (R)-2-((1-(3-cyano-7-methyl-4-oxo-2-(2-oxa-6-azaspiro[3.3]heptan-6- |
| | yl)-4H-pyrido[1,2-a]pyrimidin-9-yl)ethyl)amino)benzoic acid |
| 11-322 | 2-(((1R)-1-(2-(6-oxa-3-azabicyclo[3.1.1]heptan-3-yl)-3-cyano-7- |
| | methyl-4-oxo-4H-pyrido[1,2-a]pyrimidin-9-yl)ethyl)amino)benzoic acid |
| 11-323 | (R)-2-((1-(3-cyano-7-methyl-4-oxo-2-(2-azaspiro[3.3]heptan-2-yl)-4H- |
| | pyrido[1,2-a]pyrimidin-9-yl)ethyl)amino)benzoic acid |
| 11-324 | 2-(((1R)-1-(3-cyano-2-(3-hydroxypiperidin-1-yl)-7-methyl-4-oxo-4H- |
| | pyrido[1,2-a]pyrimidin-9-yl)ethyl)amino)benzoic acid |
| 11-325 | (R)-2-chloro-5-((1-(3-cyano-2-(isoindolin-2-yl)-7-methyl-4-oxo-4H- |
| | pyrido[1,2-a]pyrimidin-9-yl)ethyl)amino)pyrimidine-4-carboxylic acid |
| 11-326 | (R)-2-((1-(3-cyano-7-cyclopropyl-2-(isoindolin-2-yl)-4-oxo-4H- |
| | pyrido[1,2-a]pyrimidin-9-yl)ethyl)amino)benzoic acid |
| 11-327 | (S)-2-((1-(3-cyano-7-cyclopropyl-2-(isoindolin-2-yl)-4-oxo-4H- |
| | pyrido[1,2-a]pyrimidin-9-yl)ethyl)amino)benzoic acid |
| 11-328 | (R)-2-((1-(3-cyano-7-ethyl-2-(isoindolin-2-yl)-4-oxo-4H-pyrido[1,2- |
| | a]pyrimidin-9-yl)ethyl)amino)benzoic acid |
| 11-329 | (S)-1-(3-cyano-7-methyl-4-oxo-2-(piperidin-1-yl)-4H-pyrido[1,2- |
| | a]pyrimidin-9-yl)ethyl3-amino-6-chloropicolinate |
| 11-330 | (R)-1-(3-cyano-7-methyl-4-oxo-2-(piperidin-1-yl)-4H-pyrido[1,2- |
| | a]pyrimidin-9-yl)ethyl3-amino-6-chloropicolinate |
| 11-331 | (R)-6-chloro-3-((1-(3-cyano-7-methyl-4-oxo-2-(piperidin-1-yl)-4H- |
| | pyrido[1,2-a]pyrimidin-9-yl)ethyl)amino)picolinic acid |
| 11-332 | 6-chloro-3-((1-(3-cyano-7-methyl-4-oxo-2-(piperidin-1-yl)-4H- |
| | pyrido[1,2-a]pyrimidin-9-yl)ethyl)amino)picolinic acid |
| 11-333 | (2-((1-(2-(isoindolin-2-yl)-7-methyl-4-oxo-4H-pyrido[1,2-a]pyrimidin-9- |
| | yl)ethyl)amino)phenyl)(methyl)phosphinicacid |
| 11-334 | 2-((1-(2-(4-(4-cyanophenyl)piperazin-1-yl)-7-methyl-4-oxo-4H- |
| | pyrido[1,2-a]pyrimidin-9-yl)ethyl)amino)benzoic acid |
| 11-335 | 2-((1-(7-methyl-4-oxo-2-(4-phenylpiperazin-1-yl)-4H-pyrido[1,2- |
| | a]pyrimidin-9-yl)ethyl)amino)benzoic acid |
| 11-336 | 2-((1-(2-(4-(cyclopropanecarbonyl)piperazin-1-yl)-7-methyl-4-oxo-4H- |
| | pyrido[1,2-a]pyrimidin-9-yl)ethyl)amino)benzoic acid |
| 11-337 | 2-((1-(2-(4-acetylpiperazin-1-yl)-7-methyl-4-oxo-4H-pyrido[1,2- |
| | a]pyrimidin-9-yl)ethyl)amino)benzoic acid |
| 11-338 | 2-((1-(2-(4-cyanoisoindolin-2-yl)-7-methyl-4-oxo-4H-pyrido[1,2- |
| | a]pyrimidin-9-yl)ethyl)amino)benzoic acid |
| 11-339 | 2-((1-(2-((3-cyanophenyl)amino)-7-methyl-4-oxo-4H-pyrido[1,2- |
| | a]pyrimidin-9-yl)ethyl)amino)benzoic acid |
| 11-340 | 2-((1-(2-(3-cyanophenoxy)-7-methyl-4-oxo-4H-pyrido[1,2-a]pyrimidin- |
| | 9-yl)ethyl)amino)benzoic acid |
| 11-341 | 2-((1-(7-methyl-4-oxo-2-phenoxy-4H-pyrido[1,2-a]pyrimidin-9- |
| | yl)ethyl)amino)benzoic acid |
| 11-342 | 2-((1-(2-(3-hydroxy-3-methylpiperidin-1-yl)-7-methyl-4-oxo-4H- |
| | pyrido[1,2-a]pyrimidin-9-yl)ethyl)amino)benzoic acid |
| 11-343 | 2-((1-(2-(3-hydroxypiperidin-1-yl)-7-methyl-4-oxo-4H-pyrido[1,2- |
| | a]pyrimidin-9-yl)ethyl)amino)benzoic acid |
| 11-344 | 2-((1-(2-(7-azabicyclo[2.2.1]heptan-7-yl)-7-methyl-4-oxo-4H- |
| | pyrido[1,2-a]pyrimidin-9-yl)ethyl)amino)benzoic acid |
| 11-345 | (7-methyl-4-oxo-2-(piperidin-1-yl)-4H-pyrido[1,2-a]pyrimidine-9- |
| | carbonyl)proline |
| 11-346 | 1-(7-methyl-4-oxo-2-(piperidin-1-yl)-4H-pyrido[1,2-a]pyrimidine-9- |
| | carbonyl)piperidine-2-carboxylic acid |
| 11-347 | 1-(7-methyl-4-oxo-2-(piperidin-1-yl)-4H-pyrido[1,2-a]pyrimidin-9- |
| | yl)piperidine-4-carboxylic acid |
| 11-348 | 2-chloro-5-((1-(2-(isoindolin-2-yl)-3,7-dimethyl-4-oxo-4H-pyrido[1,2- |
| | a]pyrimidin-9-yl)ethyl)amino)isonicotinic acid |
| 11-349 | 2-((1-(3-cyano-2-(isoindolin-2-yl)-7-methyl-4-oxo-4H-pyrido[1,2- |
| | a]pyrimidin-9-yl)ethyl)amino)benzoic acid |
| 11-350 | 4-((1-(2-(isoindolin-2-yl)-3,7-dimethyl-4-oxo-4H-pyrido[1,2-a]pyrimidin- |
| | 9-yl)ethyl)amino)nicotinic acid |
| 11-351 | (R)-6-fluoro-3-((1-(2-(isoindolin-2-yl)-3,7-dimethyl-4-oxo-4H- |
| | pyrido[1,2-a]pyrimidin-9-yl)ethyl)amino)picolinic acid |
| 11-352 | 6-fluoro-3-((1-(2-(isoindolin-2-yl)-3,7-dimethyl-4-oxo-4H-pyrido[1,2- |
| | a]pyrimidin-9-yl)ethyl)amino)picolinic acid |
| 11-353 | (S)-3-((1-(2-(isoindolin-2-yl)-3,7-dimethyl-4-oxo-4H-pyrido[1,2- |
| | a]pyrimidin-9-yl)ethyl)amino)-6-(trifluoromethyl)picolinic acid |
| 11-354 | (R)-3-((1-(2-(isoindolin-2-yl)-3,7-dimethyl-4-oxo-4H-pyrido[1,2- |
| | a]pyrimidin-9-yl)ethyl)amino)-6-(trifluoromethyl)picolinic acid |
| 11-355 | 3-((1-(2-(isoindolin-2-yl)-3,7-dimethyl-4-oxo-4H-pyrido[1,2-a]pyrimidin- |
| | 9-yl)ethyl)amino)-6-(trifluoromethyl)picolinic acid |
| 11-356 | 3-((1-(3,7-dimethyl-4-oxo-2-(piperidin-1-yl)-4H-pyrido[1,2-a]pyrimidin- |
| | 9-yl)ethyl)amino)picolinic acid |
| 11-357 | (S)-6-chloro-3-((1-(3,7-dimethyl-4-oxo-2-(piperidin-1-yl)-4H-pyrido[1,2- |
| | a]pyrimidin-9-yl)ethyl)amino)picolinic acid |
| 11-358 | (R)-6-chloro-3-((1-(3,7-dimethyl-4-oxo-2-(piperidin-1-yl)-4H-pyrido[1,2- |
| | a]pyrimidin-9-yl)ethyl)amino)picolinic acid |
| 11-359 | 6-chloro-3-((1-(3,7-dimethyl-4-oxo-2-(piperidin-1-yl)-4H-pyrido[1,2- |
| | a]pyrimidin-9-yl)ethyl)amino)picolinic acid |
| 11-360 | (R)-6-chloro-3-((1-(2-(isoindolin-2-yl)-3,7-dimethyl-4-oxo-4H- |
| | pyrido[1,2-a]pyrimidin-9-yl)ethyl)amino)picolinic acid |
| 11-361 | 6-chloro-3-((1-(2-(isoindolin-2-yl)-3,7-dimethyl-4-oxo-4H-pyrido[1,2- |
| | a]pyrimidin-9-yl)ethyl)amino)picolinic acid |
| 11-362 | 2-((1-(3,7-dimethyl-4-oxo-2-(1-oxoisoindolin-2-yl)-4H-pyrido[1,2- |
| | a]pyrimidin-9-yl)ethyl)amino)benzoic acid |
| 11-363 | 3-fluoro-2-((1-(2-(isoindolin-2-yl)-3,7-dimethyl-4-oxo-4H-pyrido[1,2- |
| | a]pyrimidin-9-yl)ethyl)amino)benzoic acid |
| 11-364 | 4-fluoro-2-((1-(2-(isoindolin-2-yl)-3,7-dimethyl-4-oxo-4H-pyrido[1,2- |
| | a]pyrimidin-9-yl)ethyl)amino)benzoic acid |
| 11-365 | (S)-5-fluoro-2-((1-(2-(isoindolin-2-yl)-3,7-dimethyl-4-oxo-4H- |
| | pyrido[1,2-a]pyrimidin-9-yl)ethyl)amino)benzoic acid |
| 11-366 | (R)-5-fluoro-2-((1-(2-(isoindolin-2-yl)-3,7-dimethyl-4-oxo-4H- |
| | pyrido[1,2-a]pyrimidin-9-yl)ethyl)amino)benzoic acid |
| 11-367 | 5-fluoro-2-((1-(2-(isoindolin-2-yl)-3,7-dimethyl-4-oxo-4H-pyrido[1,2- |
| | a]pyrimidin-9-yl)ethyl)amino)benzoic acid |
| 11-368 | 2-fluoro-6-((1-(2-(isoindolin-2-yl)-3,7-dimethyl-4-oxo-4H-pyrido[1,2- |
| | a]pyrimidin-9-yl)ethyl)amino)benzoic acid |
| 11-370 | 2-((1-(7-methyl-4-oxo-2-((tetrahydrofuran-3-yl)thio)-4H-pyrido[1,2- |
| | a]pyrimidin-9-yl)ethyl)amino)benzoic acid |
| 11-371 | 2-((1-(7-methyl-4-oxo-2-((tetrahydrofuran-3-yl)oxy)-4H-pyrido[1,2- |
| | a]pyrimidin-9-yl)ethyl)amino)benzoic acid |
| 11-372 | 2-((1-(2-methoxy-7-methyl-4-oxo-4H-pyrido[1,2-a]pyrimidin-9- |
| | yl)ethyl)amino)benzoic acid |
| 11-373 | 2-((1-(7-methyl-4-oxo-2-(1H-pyrazol-4-yl)-4H-pyrido[1,2-a]pyrimidin-9- |
| | yl)ethyl)amino)benzoic acid |
| 11-374 | 2-(((S)-1-(7-methyl-2-((S)-1-methyl-2-oxopiperidin-4-yl)-4-oxo-4H- |
| | pyrido[1,2-a]pyrimidin-9-yl)ethyl)amino)benzoic acid |
| 11-375 | 2-(((S)-1-(7-methyl-2-((R)-1-methyl-2-oxopiperidin-4-yl)-4-oxo-4H- |
| | pyrido[1,2-a]pyrimidin-9-yl)ethyl)amino)benzoic acid |
| 11-376 | 2-(((R)-1-(7-methyl-2-((S)-1-methyl-2-oxopiperidin-4-yl)-4-oxo-4H- |
| | pyrido[1,2-a]pyrimidin-9-yl)ethyl)amino)benzoic acid |
| 11-377 | 2-((1-(7-methyl-2-(1-methyl-2-oxopiperidin-4-yl)-4-oxo-4H-pyrido[1,2- |
| | a]pyrimidin-9-yl)ethyl)amino)benzoic acid |
| 11-378 | 2-((1-(7-methyl-4-oxo-2-(1,2,3,6-tetrahydropyridin-4-yl)-4H-pyrido[1,2- |
| | a]pyrimidin-9-yl)ethyl)amino)benzoic acid |
| 11-379 | (R)-2-((1-(2-(1-acetyl-1,2,3,6-tetrahydropyridin-4-yl)-7-methyl-4-oxo- |
| | 4H-pyrido[1,2-a]pyrimidin-9-yl)ethyl)amino)benzoic acid |
| 11-380 | 2-((1-(7-methyl-4-oxo-2-(piperidin-4-yl)-4H-pyrido[1,2-a]pyrimidin-9- |
| | yl)ethyl)amino)benzoic acid |
| 11-381 | 2-((1-(2-(1-acetylpiperidin-4-yl)-7-methyl-4-oxo-4H-pyrido[1,2- |
| | a]pyrimidin-9-yl)ethyl)amino)benzoic acid |
| 11-382 | 2-((1-(7-methyl-2-(1-methyl-1,4,5,7-tetrahydro-6H-pyrazolo[3,4- |
| | c]pyridin-6-yl)-4-oxo-4H-pyrido[1,2-a]pyrimidin-9-yl)ethyl)amino)benzoic acid |
| 11-383 | 2-((1-(7-methyl-2-(1-methyl-6-oxo-1,6-dihydropyridin-3-yl)-4-oxo-4H- |
| | pyrido[1,2-a]pyrimidin-9-yl)ethyl)amino)benzoic acid |
| 11-384 | 2-((1-(7-methyl-2-(4-methyl-3-oxopiperazin-1-yl)-4-oxo-4H-pyrido[1,2- |
| | a]pyrimidin-9-yl)ethyl)amino)benzoic acid |
| 11-385 | 3-((1-(7-methyl-4-oxo-2-(piperidin-1-yl)-4H-pyrido[1,2-a]pyrimidin-9- |
| | yl)ethyl)amino)cyclobutane-1-carboxylic acid |
| 11-386 | 3-((1-(7-methyl-4-oxo-2-(piperidin-1-yl)-4H-pyrido[1,2-a]pyrimidin-9- |
| | yl)ethyl)amino)propanoic acid |
| 11-387 | 3-methyl-3-((1-(7-methyl-4-oxo-2-(piperidin-1-yl)-4H-pyrido[1,2- |
| | a]pyrimidin-9-yl)ethyl)amino)butanoic acid |
| 11-388 | 4-((1-(7-methyl-4-oxo-2-(piperidin-1-yl)-4H-pyrido[1,2-a]pyrimidin-9- |
| | yl)ethyl)amino)benzoic acid |
| 11-389 | 3-((1-(7-methyl-4-oxo-2-(piperidin-1-yl)-4H-pyrido[1,2-a]pyrimidin-9- |
| | yl)ethyl)amino)benzoic acid |
| 11-390 | 2-methyl-6-((1-(7-methyl-4-oxo-2-(piperidin-1-yl)-4H-pyrido[1,2- |
| | a]pyrimidin-9-yl)ethyl)amino)benzoic acid |
| 11-391 | 2-((1-(2-(4-fluorophenyl)-7-methyl-4-oxo-4H-pyrido[1,2-a]pyrimidin-9- |
| | yl)ethyl)amino)benzoic acid |
| 11-392 | 2-((1-(2-(4-methoxyphenyl)-7-methyl-4-oxo-4H-pyrido[1,2-a]pyrimidin- |
| | 9-yl)ethyl)amino)benzoic acid |
| 11-393 | 2-((1-(2-(3-methoxyphenyl)-7-methyl-4-oxo-4H-pyrido[1,2-a]pyrimidin- |
| | 9-yl)ethyl)amino)benzoic acid |
| 11-394 | 2-((1-(2-(2-methoxyphenyl)-7-methyl-4-oxo-4H-pyrido[1,2-a]pyrimidin- |
| | 9-yl)ethyl)amino)benzoic acid |
| 11-395 | 2-((1-(7-methyl-4-oxo-2-(o-tolyl)-4H-pyrido[1,2-a]pyrimidin-9- |
| | yl)ethyl)amino)benzoic acid |
| 11-396 | 2-((1-(2-(4-chlorophenyl)-7-methyl-4-oxo-4H-pyrido[1,2-a]pyrimidin-9- |
| | yl)ethyl)amino)benzoic acid |
| 11-397 | 2-((1-(2-(4-cyanophenyl)-7-methyl-4-oxo-4H-pyrido[1,2-a]pyrimidin-9- |
| | yl)ethyl)amino)benzoic acid |
| 11-398 | 2-((1-(2-(3-cyanophenyl)-7-methyl-4-oxo-4H-pyrido[1,2-a]pyrimidin-9- |
| | yl)ethyl)amino)benzoic acid |
| 11-399 | 2-((1-(2-(2-cyanophenyl)-7-methyl-4-oxo-4H-pyrido[1,2-a]pyrimidin-9- |
| | yl)ethyl)amino)benzoic acid |
| 11-400 | 2-((1-(7-methyl-4-oxo-2-(pyridin-4-yl)-4H-pyrido[1,2-a]pyrimidin-9- |
| | yl)ethyl)amino)benzoic acid |
| 11-401 | 2-((1-(7-methyl-4-oxo-2-(pyridin-3-yl)-4H-pyrido[1,2-a]pyrimidin-9- |
| | yl)ethyl)amino)benzoic acid |
| 11-402 | 2-((1-(7-methyl-4-oxo-2-(pyridin-2-yl)-4H-pyrido[1,2-a]pyrimidin-9- |
| | yl)ethyl)amino)benzoic acid |
| 11-403 | 2-((1-(3,7-dimethyl-4-oxo-2-phenyl-4H-pyrido[1,2-a]pyrimidin-9- |
| | yl)ethyl)amino)benzoic acid |
| 11-404 | 2-((1-(7-methyl-2-(1-methyl-1H-pyrazol-3-yl)-4-oxo-4H-pyrido[1,2- |
| | a]pyrimidin-9-yl)ethyl)amino)benzoic acid |
| 11-405 | 2-((1-(7-methyl-2-(1-methyl-1H-pyrazol-4-yl)-4-oxo-4H-pyrido[1,2- |
| | a]pyrimidin-9-yl)ethyl)amino)benzoic acid |
| 11-406 | (S)-2-((1-(2-(isoindolin-2-yl)-7-methyl-4-oxo-4H-pyrido[1,2-a]pyrimidin- |
| | 9-yl)ethyl)amino)benzoic acid |
| 11-407 | 2-((1-(2-(azetidin-1-yl)-7-methyl-4-oxo-4H-pyrido[1,2-a]pyrimidin-9- |
| | yl)ethyl)amino)benzoic acid |
| 11-408 | 2-((1-(2-(dimethylamino)-7-methyl-4-oxo-4H-pyrido[1,2-a]pyrimidin-9- |
| | yl)ethyl)amino)benzoic acid |
| 11-409 | 7-methyl-4-oxo-2-(piperidin-1-yl)-4H-pyrido[1,2-a]pyrimidine-9- |
| | carbonitrile |
| 11-410 | 9-(1-hydroxypropyl)-7-methyl-2-(piperidin-1-yl)-4H-pyrido[1,2- |
| | a]pyrimidin-4-one |
| 11-411 | 2-((1-(7-methyl-4-oxo-2-(piperidin-1-yl)-4H-pyrido[1,2-a]pyrimidin-9- |
| | yl)propyl)amino)benzoic acid |
| 11-412 | 2-(((7-methyl-4-oxo-2-(piperidin-1-yl)-4H-pyrido[1,2-a]pyrimidin-9- |
| | yl)methyl)amino)benzoic acid |
| 11-413 | 2-((1-(3-cyclopropyl-7-methyl-4-oxo-2-(piperidin-1-yl)-4H-pyrido[1,2- |
| | a]pyrimidin-9-yl)ethyl)amino)benzoic acid |
| 11-414 | (S)-2-((1-(7-methyl-4-oxo-2-(piperidin-1-yl)-3-(trifluoromethyl)-4H- |
| | pyrido[1,2-a]pyrimidin-9-yl)ethyl)amino)benzoic acid |
| 11-415 | 2-((1-(7-methyl-4-oxo-2-(piperidin-1-yl)-3-(trifluoromethyl)-4H- |
| | pyrido[1,2-a]pyrimidin-9-yl)ethyl)amino)benzoic acid |
| 11-416 | 2-((1-(4-oxo-2-(piperidin-1-yl)-7-(trifluoromethyl)-4H-pyrido[1,2- |
| | a]pyrimidin-9-yl)ethyl)amino)benzoic acid |
| 11-417 | (S)-2-((1-(7-cyano-2-(4,4-difluoropiperidin-1-yl)-4-oxo-4H-pyrido[1,2- |
| | a]pyrimidin-9-yl)ethyl)amino)benzoic acid |
| 11-418 | (R)-2-((1-(7-cyano-2-(4,4-difluoropiperidin-1-yl)-4-oxo-4H-pyrido[1,2- |
| | a]pyrimidin-9-yl)ethyl)amino)benzoic acid |
| 11-419 | 2-((1-(7-cyano-2-(4,4-difluoropiperidin-1-yl)-4-oxo-4H-pyrido[1,2- |
| | a]pyrimidin-9-yl)ethyl)amino)benzoic acid |
| 11-420 | 2-((1-(7-methyl-2-morpholino-4-oxo-4H-pyrido[1,2-a]pyrimidin-9- |
| | yl)ethyl)amino)benzoic acid |
| 11-421 | 7-methyl-2-morpholino-9-(1-(phenylamino)ethyl)-4H-pyrido[1,2- |
| | a]pyrimidin-4-one |
| 11-422 | (R)-2-((1-(4-oxo-2-(piperidin-1-yl)-4H-pyrido[1,2-a]pyrimidin-9- |
| | yl)ethyl)amino)benzoic acid |
| 11-423 | (S)-2-((1-(4-oxo-2-(piperidin-1-yl)-4H-pyrido[1,2-a]pyrimidin-9- |
| | yl)ethyl)amino)benzoic acid |
| 11-424 | 6-(2-(isoindolin-2-yl)-7-methyl-4-oxo-4H-pyrido[1,2-a]pyrimidin-9-yl)-6- |
| | azaspiro[3.4]octane-8-carboxylic acid |
| 11-425 | 1-(2-(isoindolin-2-yl)-7-methyl-4-oxo-4H-pyrido[1,2-a]pyrimidin-9- |
| | yl)azetidine-3-carboxylic acid |
| 11-426 | 2-(1-(2-(isoindolin-2-yl)-7-methyl-4-oxo-4H-pyrido[1,2-a]pyrimidin-9- |
| | yl)pyrrolidin-3-yl)acetic acid |
| 11-427 | 1-(2-(isoindolin-2-yl)-7-methyl-4-oxo-4H-pyrido[1,2-a]pyrimidin-9-yl)-4- |
| | methyl-5-oxopiperazine-2-carboxylic acid |
| 11-428 | (3aR,5r,6aS)-2-(2-(isoindolin-2-yl)-7-methyl-4-oxo-4H-pyrido[1,2- |
| | a]pyrimidin-9-yl)octahydrocyclopenta[c]pyrrole-5-carboxylic acid |
| 11-429 | (R)-2-((1-(2-(5,6-dihydroimidazo[1,2-a]pyrazin-7(8H)-yl)-3,7-dimethyl- |
| | 4-oxo-4H-pyrido[1,2-a]pyrimidin-9-yl)ethyl)amino)benzoic acid |
| 11-430 | (R)-2-((1-(2-(5,6-dihydroimidazo[1,5-a]pyrazin-7(8H)-yl)-3,7-dimethyl- |
| | 4-oxo-4H-pyrido[1,2-a]pyrimidin-9-yl)ethyl)amino)benzoic acid |
| 11-431 | (R)-2-((1-(2-(6,7-dihydropyrazolo[1,5-a]pyrazin-5(4H)-yl)-3,7-dimethyl- |
| | 4-oxo-4H-pyrido[1,2-a]pyrimidin-9-yl)ethyl)amino)benzoic acid |
| 11-432 | (R)-2-((1-(2-(4-(1H-pyrazol-1-yl)piperidin-1-yl)-3,7-dimethyl-4-oxo-4H- |
| | pyrido[1,2-a]pyrimidin-9-yl)ethyl)amino)benzoic acid |
| 11-433 | (R)-2-((1-(3,7-dimethyl-4-oxo-2-(2-(trifluoromethyl)-5,6- |
| | dihydroimidazo[1,2-a]pyrazin-7(8H)-yl)-4H-pyrido[1,2-a]pyrimidin-9- |
| | yl)ethyl)amino)benzoic acid |
| 11-434 | (R)-2-((1-(3,7-dimethyl-4-oxo-2-(4-(5-(trifluoromethyl)pyridin-2- |
| | yl)piperazin-1-yl)-4H-pyrido[1,2-a]pyrimidin-9-yl)ethyl)amino)benzoic acid |
| 11-435 | (R)-2-((1-(3,7-dimethyl-4-oxo-2-(4-(5-(trifluoromethyl)benzo[d]thiazol- |
| | 2-yl)piperazin-1-yl)-4H-pyrido[1,2-a]pyrimidin-9-yl)ethyl)amino)benzoic acid |
| 11-436 | 2-(((1R)-1-(3,7-dimethyl-4-oxo-2-(1,1,2,2-tetrafluoro-6- |
| | azaspiro[3.4]octan-6-yl)-4H-pyrido[1,2-a]pyrimidin-9-yl)ethyl)amino)benzoic |
| | acid |
| 11-437 | (R)-2-((1-(2-(2,2-difluoro-6-azaspiro[3.4]octan-6-yl)-3,7-dimethyl-4- |
| | oxo-4H-pyrido[1,2-a]pyrimidin-9-yl)ethyl)amino)benzoic acid |
| 11-438 | (R)-2-((1-(2-(4-(3-cyano-2-methylphenyl)piperazin-1-yl)-3,7-dimethyl- |
| | 4-oxo-4H-pyrido[1,2-a]pyrimidin-9-yl)ethyl)amino)benzoic acid |
| 11-439 | (R)-2-((1-(3,7-dimethyl-4-oxo-2-(4-(3-(trifluoromethyl)phenyl)piperazin- |
| | 1-yl)-4H-pyrido[1,2-a]pyrimidin-9-yl)ethyl)amino)benzoic acid |
| 11-440 | (R)-2-((1-(2-(4-(2-fluorophenyl)piperazin-1-yl)-3,7-dimethyl-4-oxo-4H- |
| | pyrido[1,2-a]pyrimidin-9-yl)ethyl)amino)benzoic acid |
| 11-441 | (R)-2-((1-(3,7-dimethyl-4-oxo-2-(4-(pyridin-4-yl)piperazin-1-yl)-4H- |
| | pyrido[1,2-a]pyrimidin-9-yl)ethyl)amino)benzoic acid |
| 11-442 | (R)-2-((1-(3,7-dimethyl-4-oxo-2-(3-(5-(trifluoromethyl)isoxazol-3- |
| | yl)azetidin-1-yl)-4H-pyrido[1,2-a]pyrimidin-9-yl)ethyl)amino)benzoic acid |
| 11-443 | 2-(((R)-1-(2-((1R,4R)-5-(2,2-difluoroethyl)-2,5- |
| | diazabicyclo[2.2.1]heptan-2-yl)-3,7-dimethyl-4-oxo-4H-pyrido[1,2- |
| | a]pyrimidin-9-yl)ethyl)amino)benzoic acid |
| 11-444 | 2-(((1R)-1-(2-(5,5-difluorohexahydrocyclopenta[c]pyrrol-2(1H)-yl)-3,7- |
| | dimethyl-4-oxo-4H-pyrido[1,2-a]pyrimidin-9-yl)ethyl)amino)benzoic acid |
| 11-445 | 2-(((R)-1-(3,7-dimethyl-4-oxo-2-((1R,5S,6R)-6-(trifluoromethyl)-3- |
| | azabicyclo[3.1.0]hexan-3-yl)-4H-pyrido[1,2-a]pyrimidin-9- |
| | yl)ethyl)amino)benzoic acid |
| 11-446 | (R)-2-((1-(3,7-dimethyl-2-(4-(4-methylthiazol-2-yl)piperazin-1-yl)-4- |
| | oxo-4H-pyrido[1,2-a]pyrimidin-9-yl)ethyl)amino)benzoic acid |
| 11-447 | 2-(((1R)-1-(2-(7,7-difluoro-2-azaspiro[4.4]nonan-2-yl)-3,7-dimethyl-4- |
| | oxo-4H-pyrido[1,2-a]pyrimidin-9-yl)ethyl)amino)benzoic acid |

or a pharmaceutically acceptable salt of one of the foregoing compounds.

### Pharmaceutical Compositions

The compounds of Formula I may be formulated into pharmaceutical compositions.

In another aspect, the invention provides pharmaceutical compositions comprising a PI3Kα inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O—alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In another aspect, the present disclosure generally relates to methods for treating cancer. These methods comprise administering to a subject in need thereof, a therapeutically effective amount of a PI3K inhibitor (e.g., PI3Kα inhibitor or PI3Kα H1047R mutant inhibitor).

In some embodiments, the PI3K inhibitor (e.g., PI3Kα inhibitor or PI3Kα H1047R mutant inhibitor) is a compound of Formula (I), (IIa), (IIb), (IIc) or a pharmaceutically acceptable salt, prodrug, solvate, hydrate, isomer, or tautomer thereof. In particular embodiments, the PI3K inhibitor comprises a compound selected from Table 1.

In another aspect, the present disclosure provides a compound obtainable by, or obtained by, a method for preparing a compound as described herein (e.g., a method comprising one or more steps described in the Schemes).

In another aspect, the present disclosure provides a pharmaceutical composition comprising a compound of Formula (I), (IIa), (IIb), (IIc), or a pharmaceutically acceptable salt, prodrug, solvate, hydrate, isomer, or tautomer thereof, and a pharmaceutically acceptable diluent or carrier.

In another aspect, the present disclosure provides an intermediate as described herein, being suitable for use in a method for preparing a compound as described herein (e.g., the intermediate is selected from the intermediates described in the Examples).

In another aspect, the present disclosure provides a method of modulating PI3K (e.g., PI3Kα) activity (e.g., in vitro or in vivo), comprising contacting a cell with a therapeutically effective amount of a compound of Formula (I), (IIa), (IIb), (IIc), or a pharmaceutically acceptable salt, prodrug, solvate, hydrate, isomer, or tautomer thereof.

In particular embodiments, the PI3K-associated with the disease or disorder has a H1047R mutation. For example, in certain embodiments as otherwise described herein, the compounds have a high selectivity for inhibiting H1047R-mutated PI3Kα compared to wild-type PI3Kα. This unexpected finding suggests that certain compounds may allow targeted inhibition of PI3Kα with a novel binding mechanism compared to conventional wild-type PI3Kα inhibitors. Without wishing to be bound by theory, the H1047R-mutated PI3Kα has a modification distant from the wild-type PI3Kα active site. Accordingly, compounds which are selective for H1047R-mutated PI3Kα over wild-type PI3Kα are not believed to strongly bind at the PI3Kα active site, but rather advantageously are believed to target other binding pockets. As the active site of PI3K-type proteins is thought to be better conserved among different variants, compounds that effectively bind at a position different than the active site may provide high selectivity for PI3Kα inhibition over other PI3K proteins, such as PI3 Kβ.

In another aspect, methods of treating cancer comprising administering to a patient having cancer a therapeutically effective amount of a compound of Formula I, pharmaceutically acceptable salts thereof or pharmaceutical compositions comprising the compound or pharmaceutically acceptable salts thereof are provided.

The compositions and methods provided herein may be used for the treatment of a wide variety of cancer including tumors such as prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal; esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is selected from hepatocellular carcinoma, breast cancer, skin cancer, bladder cancer, liver cancer, pancreatic cancer, and head and neck cancer,

In another embodiment, the cancer is selected from breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

Thus, in certain embodiments, this disclosure provides methods of treating cancer comprising administering a compound of Formula (I) a pharmaceutical composition thereof and KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In other embodiments, this disclosure provides a methods of treating cancer comprising administering a compound of Formula (I) a pharmaceutical composition thereof and a mutant selective KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

In another embodiment, this disclosure provides a compound of Formula (I) a pharmaceutical composition thereof for use in the treatment of cancer in combination with a KRAS inhibitor, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

The present disclosure provides methods of treating, preventing, or ameliorating a disease or disorder in which PI3K plays a role by administering to a patient in need thereof a therapeutically effective amount of a PI3K inhibitor. The methods of the present disclosure can be used in the treatment of a variety of PI3K-dependent diseases and disorders.

In some embodiments, the disease of disorder is a cancer (e.g., breast cancer, brain cancers, prostate cancer, endometrial cancer, gastric cancer, leukemia, lymphoma, sarcoma, colorectal cancer, lung cancer, ovarian cancer, skin cancer, and head and neck cancer). In some embodiments, the disease or disorder associated with PI3K includes, but is not limited to, CLOVES syndrome (congenital lipomatous overgrowth, vascular malformations, epidermal naevi, scoliosis/skeletal and spinal syndrome), PIK3CA-related overgrowth syndrome (PROS), endometrial cancer, breast cancer, esophageal squamous-cell cancer, cervical squamous-cell carcinoma, cervical adenocarcinoma, colorectal adenocarcinoma, bladder urothelial carcinoma, glioblastoma, ovarian cancer, non-small-cell lung cancer, esophagogastric cancer, nerve-sheath tumor, head and neck squamous-cell carcinoma, melanoma, esophagogastric adenocarcinoma, soft-tissue sarcoma, prostate cancer, fibrolamellar carcinoma, hepatocellular carcinoma, diffuse glioma, colorectal cancer, pancreatic cancer, cholangiocarcinoma, B-cell lymphoma, mesothelioma, adrenocortical carcinoma, renal non-clear-cell carcinoma, renal clear-cell carcinoma, germ-cell carcinoma, thymic tumor, pheochromocytoma, miscellaneous neuroepithelial tumor, thyroid cancer, leukemia, and encapsulated glioma.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### GENERAL REACTION SCHEMES, INTERMEDIATES AND EXAMPLES

### General Reaction Schemes

The compounds of the present invention may be prepared using commercially available reagents and intermediates in the synthetic methods and reaction schemes described herein, or may be prepared using other reagents and conventional methods well known to those skilled in the art.

Where reference is made to “first eluting enantiomer” or “second eluting enantiomer”, unless otherwise specifically indicated, the specific stereochemistry of such enantiomers has not been determined and any stereochemistry depicted in the corresponding compound structures has been arbitrarily assigned.

For instance, intermediates for preparing compounds and compounds of Formula (I) of the present invention may be prepared according to General Reaction Schemes I-III:

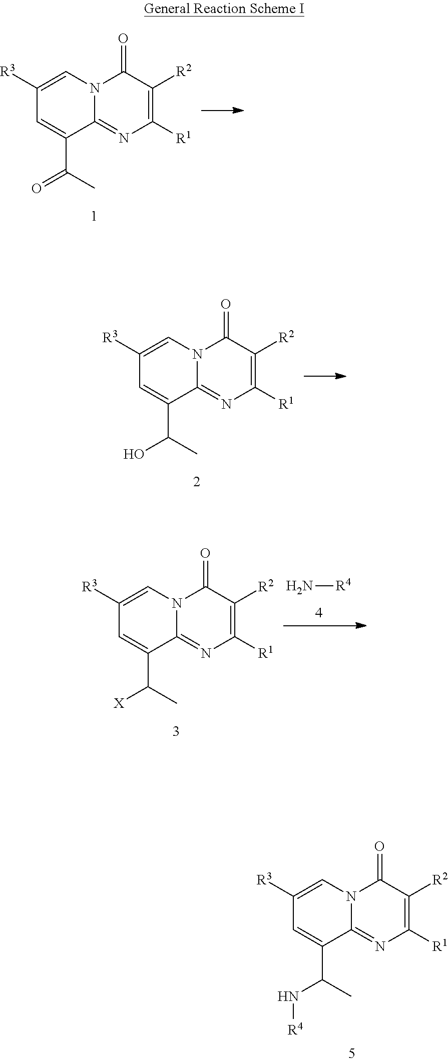

For General Reaction Scheme I, Compound 5 is an example of Formula (I). In this General Reaction Scheme I, 1 is reacted with a reducing agent such as sodium borohydride, to yield Compound 2. Compound 2 can then undergo a halogenation reaction with a reagent such as phosphorus tribromide to form the halogenated product 3. Compound 3 could be then be treated with a nucleophilic amine 4, this reaction could, for example, be a nucleophilic substitution or a metal catalyzed reaction, in the presence of a suitable base, e.g., N,N-diisopropylethylamine, to form title compound 5.

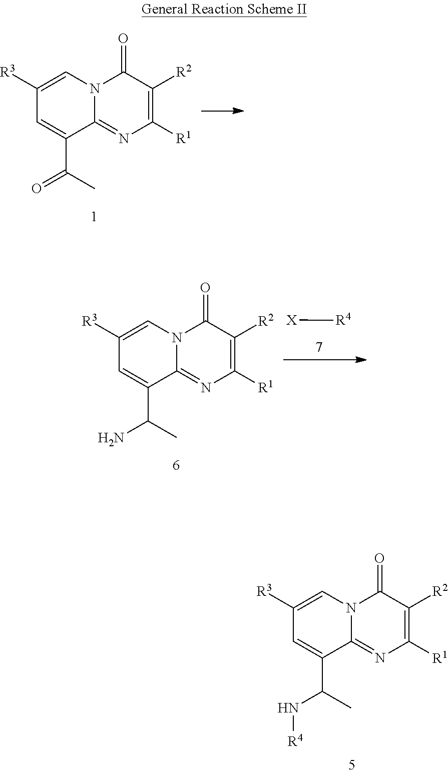

For General Reaction Scheme II, Compound 5 is an example of Formula (I). In this General Reaction Scheme II, 1 is reacted with a reducing agent, for example sodium cyanoborohydride, in the presence of an ammonium salt, such as ammonium acetate, to yield Compound 6. Compound 6 can then undergo a metal catalyzed reaction or nucleophilic substitution with a coupling partner, such as a halide, X—R⁴ 7 in the presence of a suitable base, e.g., sodium carbonate, to form title compound 5.

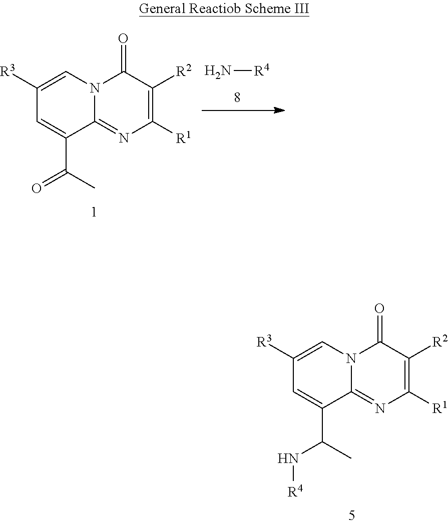

For General Reaction Scheme III, Compound 5 is an example of Formula (I). In this General Reaction Scheme II, 1 is reacted with a reducing agent, for example sodium cyanoborohydride, in the presence of a substituted amine 8, NH₂-R⁴, to yield Compound 5.

### Intermediate A

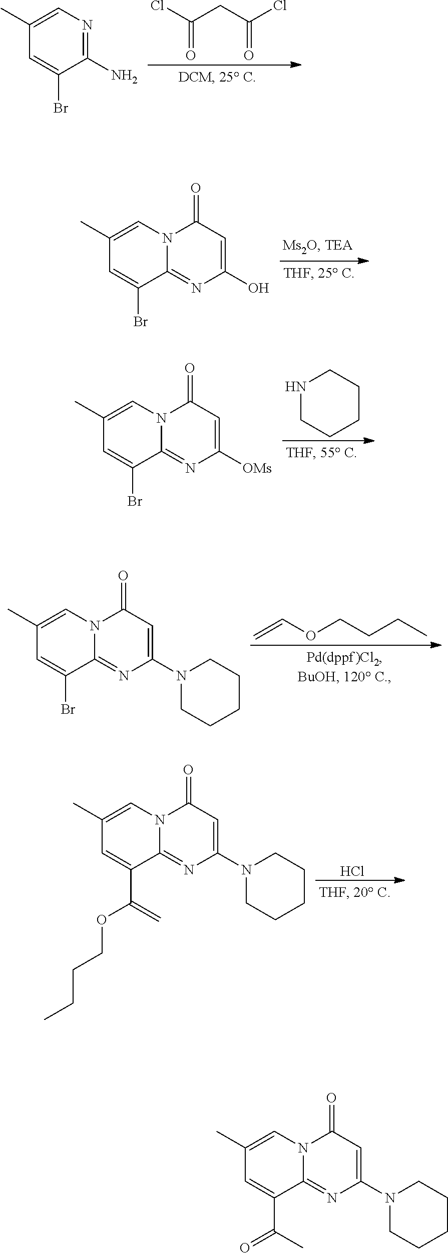

Step A: To a solution of 3-bromo-5-methyl-pyridin-2-amine (10.0 g, 53.5 mmol, 1.00 eq.) in dichloromethane (100 mL) was added malonyl chloride (7.91 g, 56.1 mmol, 5.46 mL, 1.05 eq.) dropwise and the reaction mixture was stirred at 25° C. for 48 hours. Upon completion, the reaction solution turned turbid, the yellow cake was collected by filtration, washed with dichloromethane (200 mL), and dried under reduced pressure to give 9-bromo-2-hydroxy-7-methyl-4H-pyrido[1,2-a]pyrimidin-4-one (11.4 g, 31.4 mmol, 58.7% yield, 70.2% purity) as a yellow solid. LCMS [M+1]⁺=254.9.

¹H NMR (400 MHz, DMSO-d₆) δ=8.72 (s, 1H), 8.28 (d, J=1.6 Hz, 1H), 5.51 (s, 1H), 2.34 (s, 3H).

Step B: To a solution of 9-bromo-2-hydroxy-7-methyl-4H-pyrido[1,2-a]pyrimidin-4-one (11.4 g, 44.7 mmol, 1.00 eq.) in tetrahydrofuran (110 mL) was added triethylamine (18.1 g, 179 mmol, 24.9 mL, 4.00 eq.) and methanesulfonic anhydride (11.7 g, 67.0 mmol, 1.50 eq.) at 0° C. The mixture was stirred at 25° C. for one hour, then piperidine (11.4 g, 134 mmol, 13.2 mL, 3.00 eq.) was added and the mixture was heated at 55° C. for 12 hours. After completion of the reaction, the mixture was cooled to 25° C. and the reaction mixture was then concentrated under reduced pressure to give a residue. The residue was purified by column chromatography (silica gel, petroleum ether/ethyl acetate=1/0 to 1/1) to give 9-bromo-7-methyl-2-(piperidin-1-yl)-4H-pyrido[1,2-a]pyrimidin-4-one (5.20 g, 13.2 mmol, 29.5% yield, 81.8% purity) as a yellow solid. LCMS [M+1]⁺=324.0.

¹H NMR (400 MHz, CDCl₃) O=8.67 (s, 1H), 7.80 (d, J=2.0 Hz, 1H), 5.61 (s, 1H), 3.69 (br s, 4H), 2.31 (d, J=1.0 Hz, 3H), 1.70 (br d, J=3.6 Hz, 2H), 1.65 (br d, J=4.8 Hz, 4H).

Step C: A mixture of 9-bromo-7-methyl-2-(piperidin-1-yl)-4H-pyrido[1,2-a]pyrimidin-4-one (5.20 g, 16.1 mmol, 1.00 eq.), n-butyl vinyl ether (4.85 g, 48.4 mmol, 6.23 mL, 3.00 eq.), diisopropylethylamine (6.26 g. 48.4 mmol, 8.43 mL, 3.00 eq.) -Bis(diphenylphosphino) ferrocene) palladium (II) dichloride (1.18 g, 1.61 mmol, 0.10 eq.) in n-butanol (50.0 mL) was degassed and purged with nitrogen 3 times, and then the mixture was stirred at 120° C. for 12 hours under a nitrogen atmosphere. The mixture was then cooled to 25° C., and to the mixture was added hydrochloric acid aqueous (3N in water, 40 mL), then stirred at 25° C. for one hour. After this time, the mixture was adjusted to pH=8 with saturated sodium bicarbonate solution. The mixture was further diluted with water (150 mL) and extracted with dichloromethane (150 mL×2). The combined organic phases were washed with water (50 mL×2), dried over anhydrous sodium sulfate, filtered, and concentrated under reduced pressure to give a residue. The residue was purified by column chromatography (Silica gel, petroleum ether/ethyl acetate=1/0 to 1/1) to give 9-acetyl-7-methyl-2-(piperidin-1-yl)-4H-pyrido[1,2-a]pyrimidin-4-one (2.7 g, 9.40 mmol, 58.2% yield, 99.3% purity) as a yellow solid.

¹H NMR (400 MHz, CDCl₃-d) δ=8.84 (d, J=0.8 Hz, 1H), 7.82 (d, J=2.4 Hz, 1H), 5.72-5.57 (m, 1H), 3.67-3.59 (m, 4H), 2.79 (s, 3H), 2.34 (s, 3H), 1.74-1.68 (m, 2H), 1.65-1.59 (m, 4H).

### Intermediate B

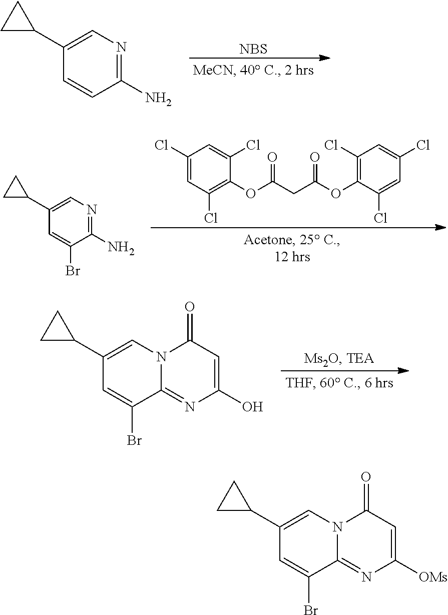

Step A: To a solution of 5-cyclopropylpyridin-2-amine (1.50 g, 11.2 mmol, 1.00 eq.) in acetonitrile (30.0 mL) was added N-bromosuccinimide (2.98 g, 16.8 mmol, 1.50 eq.), and the mixture was stirred at 40° C. for one hour. The mixture was diluted with water (10.0 mL) and extracted with ethyl acetate (10.0 mL×3), and the combined organic phases were dried with anhydrous sodium sulfate, filtered, and concentrated under reduced pressure. The residue was then purified by silica gel chromatography (petroleum ether/ethyl acetate=50/1-3/1) to get 3-bromo-5-cyclopropylpyridin-2-amine (0.89 g, 4.14 mmol, 37.0% yield, 99% purity) as yellow solid. LCMS [M+3]⁺=214.7.

¹H NMR (400 MHz, DMSO-d₆) δ=7.79 (d, J=2.0 Hz, 1H), 7.39 (d, J=2.0 Hz, 1H), 5.91 (s, 2H), 1.82-1.72 (m, 1H), 0.86-0.80 (m, 2H), 0.60-0.55 (m, 2H).

Step B: To a solution of 3-bromo-5-cyclopropylpyridin-2-amine (490 mg, 2.30 mmol, 1.00 eq.) in acetone (5.00 mL) was added bis(2,4,6-trichlorophenyl) malonate (1.06 g, 2.30 mmol, 1.00 eq.). The mixture was stirred at 25° C. for 12 hours, and the reaction mixture was cooled to 20° C. and the resulting precipitate was filtered. The filter cake was washed with acetone (5.00 mL) to give 9-bromo-7-cyclopropyl-2-hydroxy-4H-pyrido[1,2-a]pyrimidin-4-one (440 mg, 1.55 mmol, 67.4% yield, 99% purity) as yellow solid. LCMS [M+3]⁺=280.9.

Step C: To a solution of 9-bromo-7-cyclopropyl-2-hydroxy-4H-pyrido[1,2-a]pyrimidin-4-one (0.3 g, 1.07 mmol, 1.00 eq.) in tetrahydrofuran (3.00 mL) was added triethylamine (324 mg, 3.20 mmol, 446 μL, 3.00 eq.) and methanesulfonic anhydride (278.86 mg, 1.60 mmol, 1.50 eq.), and the mixture was stirred at 60° C. for 6 hours. The reaction mixture was concentrated under reduced pressure to get 9-bromo-7-cyclopropyl-4-oxo-4H-pyrido[1,2-a]pyrimidin-2-yl methanesulfonate (385 mg, crude) as yellow solid.

The following Examples are intended to illustrate further certain embodiments of the invention and are not intended to limit the scope of the invention.

### Example 1

### 7-methyl-9-(1-(phenylamino)ethyl)-2-(piperidin-1-yl)-4H-pyrido[1,2-a]pyrimidin-4-one

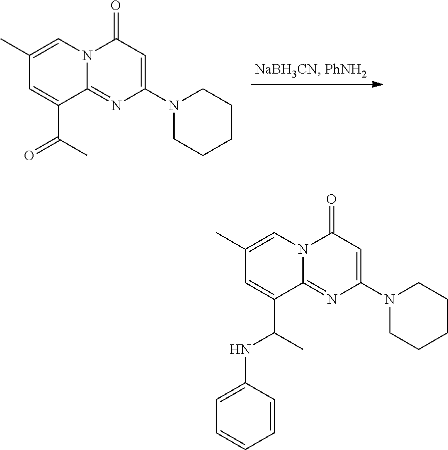 [SMILES: CC(=O)c1cc(C)cn2c(=O)cc(N3CCCCC3)nc12 ; Cc1cc(C(C)Nc2ccccc2)c2nc(N3CCCCC3)cc(=O)n2c1]

To a solution of 9-acetyl-7-methyl-2-(1-piperidyl) pyrido[1,2-a]pyrimidin-4-one (100 mg, 350 μmol, 1.00 eq.), aniline (32.6 mg, 350 μmol, 32.0 μL, 1.00 eq.) in methanol (1.00 mL) was added acetic acid (21.0 mg, 350 μmol, 20.0 μL, 1.00 eq.), then mixture was heated to 50° C. and stirred at 50° C. for 2 hours. The mixture was then cooled to 25° C. and sodium cyanoborohydride (66.0 mg, 1.05 mmol, 3.00 eq.) was added and stirred at 40° C. for another two hours. After completion of the reaction, the mixture was cooled to 25° C., the mixture was poured into saturated sodium bicarbonate aqueous solution (10.0 mL) and extracted with ethyl acetate (20.0 mL×2). The combined organic layers were washed with brine (10.0 mL), dried over anhydrous sodium sulfate, then the mixture was filtered and the filtrate was concentrated to give a residue. The residue was purified by prep-HPLC (Phenomenex luna C18 150×25 mm×10 um; mobile phase: phase A: 0.225% formic acid in water, phase B: acetonitrile; B %: 54%-84%) to give 7-methyl-9-(1-(phenylamino)ethyl)-2-(piperidin-1-yl)-4H-pyrido[1,2-a]pyrimidin-4-one (25 mg, 68.0 μmol, 19.4% yield, 98.6% purity) as a yellow solid. LCMS [M+1]⁺=363.2.

¹H NMR (400 MHz, DMSO-de) 0=8.49 (s, 1H), 7.59 (d, J=2.0 Hz, 1H), 6.99 (t, J=7.8 Hz, 2H), 6.48 (t, J=7.2 Hz, 1H), 6.43 (d, J=7.6 Hz, 2H), 6.25 (d, J=6.8 Hz, 1H), 5.61 (s, 1H), 5.09 (quin, J=6.4 Hz, 1H), 3.66 (br t, J=4.8 Hz, 4H), 2.22 (s, 3H), 1.68-1.61 (m, 2H), 1.56 (br d, J=3.6 Hz, 4H), 1.46 (d, J=6.8 Hz, 3H).

### Example 2

### 2-((1-(7-methyl-4-oxo-2-(piperidin-1-yl)-4H-pyrido[1,2-a]pyrimidin-9-yl)ethyl)amino) benzoic acid

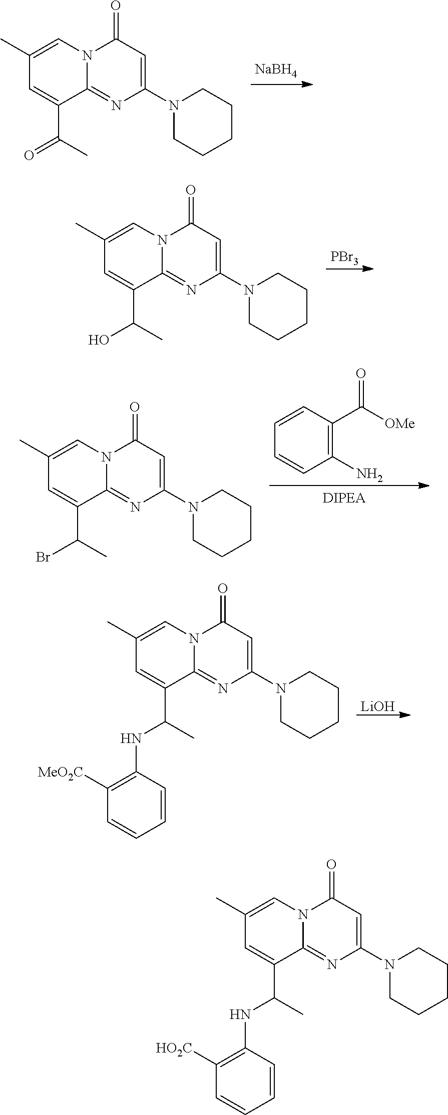

Step A: To a solution of 9-acetyl-7-methyl-2-(piperidin-1-yl)-4H-pyrido[1,2-a]pyrimidin-4-one (360 mg, 1.26 mmol, 1.00 eq.) in dichloromethane (5.00 mL) was added sodium borohydride (95.5 mg, 2.52 mmol, 2.00 eq.). The mixture was stirred at 0° C. for one hour, then diluted with saturated ammonium chloride solution (1.00 mL) at 0° C. under a nitrogen atmosphere. The solution was further diluted with water (15.0 mL) and extracted with dichloromethane (80 mL×2). The combined organic phases were washed with brine (50 mL), dried over anhydrous sodium sulfate, filtered, and concentrated under reduced pressure to give a residue. The residue was then purified by column chromatography (SiO₂, petroleum ether/ethyl acetate=1/1 to 0/1) to give 9-(1-hydroxyethyl)-7-methyl-2-(piperidin-1-yl)-4H-pyrido[1,2-a]pyrimidin-4-one (230 mg, 728 μmol, 57.7% yield, 91.0% purity) as a yellow solid. LCMS [M+1]⁺=288.2.

Step B: To a solution of 9-(1-hydroxyethyl)-7-methyl-2-(piperidin-1-yl)-4H-pyrido[1,2-a]pyrimidin-4-one (166 mg, 578 μmol, 1.00 eq.) in dichloromethane (2.00 mL) was added phosphorus tribromide (164 mg, 607 μmol, 1.05 eq.), and the mixture was stirred at 25° C. for 30 minutes. The mixture was poured into saturated sodium bicarbonate aqueous solution (10.0 mL) and extracted with dichloromethane (30.0 mL×2). The combined organic layers were washed with brine (20 mL), dried over anhydrous sodium sulfate, then the mixture was filtered and the filtrate was concentrated to give 9-(1-bromoethyl)-7-methyl-2-(piperidin-1-yl)-4H-pyrido[1,2-a]pyrimidin-4-one (200 mg, crude) as a yellow oil which was used directly.

Step C: To a solution of 9-(1-bromoethyl)-7-methyl-2-(piperidin-1-yl)-4H-pyrido[1,2-a]pyrimidin-4-one (199 mg, 568.16 μmol, 1.00 eq.), methyl 2-aminobenzoate (258 mg, 1.70 mmol, 220 μL, 3.00 eq.) in dimethylformamide (2.00 mL) was added diisopropylethylamine (220 mg, 1.70 mmol, 297 μL, 3.00 eq.) and the mixture was stirred at 80° C. for 12 hours. After completion of the reaction, the mixture was cooled to 25° C., and the pH of the mixture was adjusted with hydrochloric acid (1N in water) aqueous solution to pH=5-6. The solution was then extracted with dichloromethane (60 mL×2), and the combined organic layers were washed with brine (50 mL), dried over anhydrous sodium sulfate and concentrated to give methyl 2-((1-(7-methyl-4-oxo-2-(piperidin-1-yl)-4H-pyrido[1,2-a]pyrimidin-9-yl)ethyl)amino)benzoate (250 mg, crude) as a yellow oil which was used directly.

Step D: To a solution of methyl 2-((1-(7-methyl-4-oxo-2-(piperidin-1-yl)-4H-pyrido[1,2-a]pyrimidin-9-yl)ethyl)amino)benzoate (250 mg, 594 μmol, 1.00 eq.) in a mixture solvent of methanol (1.00 mL) and tetrahydrofuran (1.00 mL), water (0.5 mL) was added lithium hydroxide (28.5 mg, 1.19 mmol, 2.00 eq.) and the mixture was stirred at 45° C. for 12 hours. After this time, the mixture was cooled to 25° C., then the pH of the mixture was adjusted to pH=5-6 with hydrochloric acid (1N in water). The solution was then extracted with dichloromethane (60.0 mL×2, and the combined organic layers were washed with brine (50.0 mL), dried over anhydrous sodium sulfate, and concentrated under reduced pressure to give a residue. The residue was purified by prep-HPLC (Phenomenex Synergi C18 150×25 mm×10 um; mobile phase: phase A: 0.225% formic acid in water, phase B: acetonitrile; B %: 51%-81%) to give 2 2-((1-(7-methyl-4-oxo-2-(piperidin-1-yl)-4H-pyrido[1,2-a]pyrimidin-9-yl)ethyl)amino)benzoic acid (15.0 mg, 36.6 μmol, 6.16% yield, 99.3% purity) as a yellow solid. LCMS [M+1]⁺=407.3.

¹H NMR (400 MHz, CDCl₃) δ=8.68 (s, 1H), 8.25 (s, 1H), 8.05 (dd, J=1.2, 8.0 Hz, 1H), 7.50 (d, J=1.6 Hz, 1H), 7.27-7.21 (m, 1H), 6.63 (t, J=7.6 Hz, 1H), 6.33 (d, J=8.4 Hz, 1H), 5.81 (s, 1H), 5.30 (quin, J=6.4 Hz, 1H), 3.71 (s, 4H), 2.26 (s, 3H), 1.80-1.67 (m, 4H), 1.66 (s, 3H), 1.65 (s, 2H).

### Example 3-1 and 3-2

### (R)-2-((1-(7-methyl-4-oxo-2-(piperidin-1-yl)-4H-pyrido[1,2-a]pyrimidin-9-yl)ethyl)amino)benzoic acid and(S)-2-((1-(7-methyl-4-oxo-2-(piperidin-1-yl)-4H-pyrido[1,2-a]pyrimidin-9-yl)ethyl)amino)benzoic acid

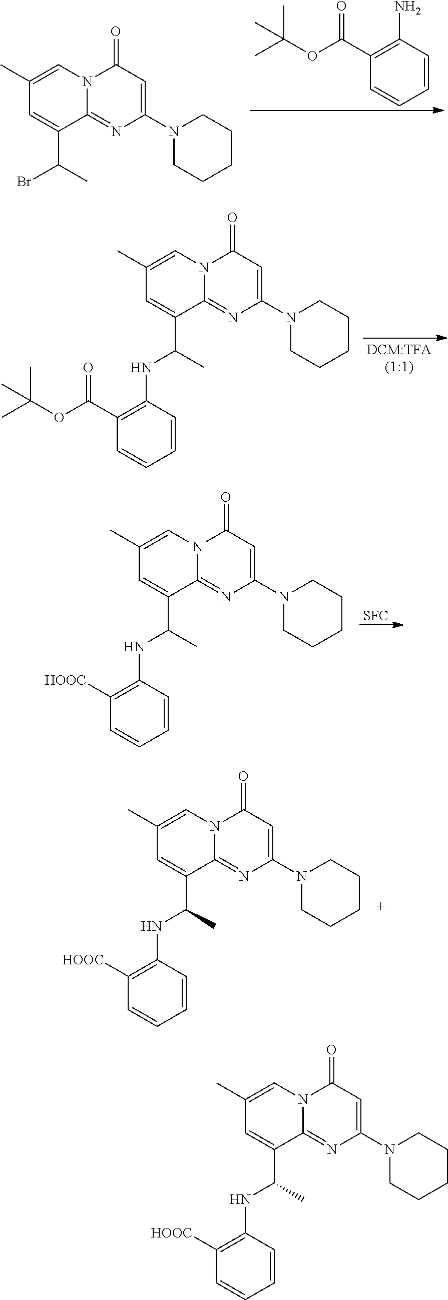

Step A: A mixture of tert-butyl 2-aminobenzoate (1.03 g, 5.34 mmol, 973 μL, 1.70 eq.) and 9-(1-bromoethyl)-7-methyl-2-(piperidin-1-yl)-4H-pyrido[1,2-a]pyrimidin-4-one (1.10 g, 3.14 mmol, 1.00 eq.) in dimethylformamide (6.00 mL) was stirred at 80° C. for 5 hours. After completion of the reaction, the mixture was cooled to 25° C. The reaction mixture was then filtered and purified by prep-HPLC (column: Phenomenex luna C18 150×40 mm×15 um; mobile phase: phase A: 0.225% formic acid in water, phase B: acetonitrile; B %: 87%-100%) to give tert-butyl 2-((1-(7-methyl-4-oxo-2-(piperidin-1-yl)-4H-pyrido[1,2-a]pyrimidin-9-yl)ethyl)amino)benzoate (500 mg, 1.08 mmol, 34.4% yield) as a light yellow solid. LCMS [M+Na]⁺=463.2.

Step B: To a solution of tert-butyl 2-((1-(7-methyl-4-oxo-2-(piperidin-1-yl)-4H-pyrido[1,2-a]pyrimidin-9-yl)ethyl)amino)benzoate (500 mg, 1.08 mmol, 1.00 eq.) in dichloromethane (3.00 mL) was added trifluoroacetic acid (1.50 mL) dropwise. The mixture was stirred at 20° C. for 16 hours, then concentrated under reduced pressure to give a residue. The residue was purified by prep-HPLC (column: Phenomenex luna C18 150×40 mm×15 um; mobile phase: [phase A: 0.225% formic acid in water, phase B: acetonitrile; B %: 51%-81%) to give 2-((1-(7-methyl-4-oxo-2-(piperidin-1-yl)-4H-pyrido[1,2-a]pyrimidin-9-yl)ethyl)amino)benzoic acid (320 mg, 787 μmol, 72.8% yield) as a white solid. LCMS [M+1]⁺=407.1.

Step C: 320 mg of 2-((1-(7-methyl-4-oxo-2-(piperidin-1-yl)-4H-pyrido[1,2-a]pyrimidin-9-yl)ethyl)amino)benzoic acid was purified by prep-HPLC (column: DAICEL CHIRALPAK IC (250 mm×30 mm, 10 um); mobile phase: phase A: acetonitrile, phase B: 0.1% ammonium hydroxide in isopropanol; B %: 50%) to give the first eluting enantiomer (130 mg, 307 μmol, 39.1% yield, 96.5% purity) as a white solid. (SFC, Rt=1.573 min). LCMS [M+1]⁺=407.2.

¹H NMR (400 MHz, CDCl₃) δ=8.67 (s, 1H), 8.21 (br d, J=1.2 Hz, 1H), 8.04 (dd, J=1.6, 8.0 Hz, 1H), 7.49 (d, J=1.6 Hz, 1H), 7.27-7.20 (m, 1H), 6.63 (t, J=7.6 Hz, 1H), 6.33 (d, J=8.8 Hz, 1H), 5.78 (s, 1H), 5.33 (br d, J=6.4 Hz, 1H), 3.70 (br s, 4H), 2.26 (s, 3H), 1.77-1.70 (m, 2H), 1.69-1.61 (m, 7H).

SFC: “Column: Column: IC-3 50×4.6 mm I.D., 3 um; Mobile phase: Phase A for CO₂, and Phase B for methanol (0.05% DEA); Gradient elution: methanol (0.05% DEA) in CO₂ from 5% to 40%, Flow rate: 3 mL/min; Detector: PDA, Column Temp: 35° C.; Back Pressure: 100 Bar”.

The second eluting enantiomer (125.9 mg, 308 μmol, 39.1% yield, 99.4% purity) as an off-white solid (SFC, Rt=2.20 min). LCMS [M+1]⁺=407.2.

¹H NMR (400 MHz, CDCl₃) δ=8.66 (s, 1H), 8.32-8.07 (m, 1H), 8.03 (d, J=8.0 Hz, 1H), 7.49 (d, J=1.6 Hz, 1H), 7.26-7.22 (m, 1H), 6.63 (t, J=7.6 Hz, 1H), 6.33 (d, J=8.8 Hz, 1H), 5.78 (s, 1H), 5.32 (br d, J=6.4 Hz, 1H), 3.70 (br s, 4H), 2.26 (s, 3H), 1.77-1.70 (m, 2H), 1.70-1.60 (m, 7H).

Characterization by SFC: “Column: Column: IC-3 50×4.6 mm I.D., 3 um; Mobile phase: Phase A for CO₂, and Phase B for methanol (0.05% DEA); Gradient elution: methanol (0.05% DEA) in CO₂ from 5% to 40%, Flow rate: 3 mL/min; Detector: PDA, Column Temp: 35° C.; Back Pressure: 100 Bar”.

The following compounds are prepared essentially according to the procedures set forth in the above schemes and examples.

| Ex. # | Structure | Spectral Data |
|---|---|---|
| 3-3 | 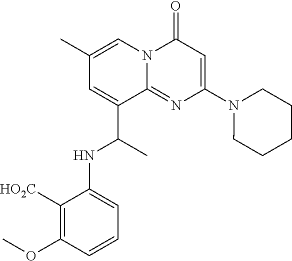 [SMILES: COc1cccc(NC(C)c2cc(C)cn3c(=O)cc(N4CCCCC4)nc23)c1C(=O)O] | ¹H NMR (400 MHz, DMSO- d₆) δ = 8.53 (s, 1H), 7.60 (d, J = 1.6 Hz, 1H), 7.29-7.07 (m, 1H), 7.02 (t, J = 8.4 Hz, 1H), 6.28 (d, J = 8.4 Hz, 1H), 5.95 (d, J = 8.4 Hz, 1H), 5.66 (s, 1H), 5.16 (q, J = 6.0Hz, 1H), 3.75 (s, 3H), 3.69 (br s, 4H), 2.25 (s, 3H), 1.68 (br d, J = 4.8 Hz, 2H), 1.60 (br d, J = 4.0 Hz, 4H), 1.53 (br d, J = 6.8 Hz, 3H). LCMS [M + 1]⁺ = 437.1. |
| | 2-methoxy-6-((1-(7-methyl-4-oxo-2-(piperidin-1- | |
| | yl)-4H-pyrido[1,2-a]pyrimidin-9- | |
| | yl)ethyl)amino)benzoic acid | |
| 3-4 | 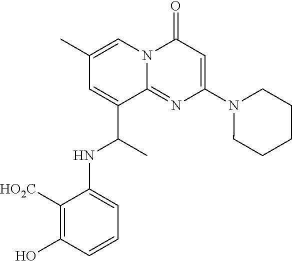 [SMILES: Cc1cc(C(C)Nc2cccc(O)c2C(=O)O)c2nc(N3CCCCC3)cc(=O)n2c1] | ¹H NMR (400 MHz, DMSO- d₆) δ = 8.50 (s, 1H), 7.54 (d, J = 1.6 Hz, 1H), 6.89 (br t, J = 8.0 Hz, 1H), 5.94 (d, J = 8.0 Hz, 1H), 5.70-5.54 (m, 2H), 5.12 (q, J = 6.4 Hz, 1H), 3.65 (br s, 4H), 2.23 (s, 3H), 1.68-1.49 (m, 9H). LCMS [M + 1]⁺ = 423.1. |
| | 2-hydroxy-6-((1-(7-methyl-4-oxo-2-(piperidin-1- | |
| | yl)-4H-pyrido[1,2-a]pyrimidin-9- | |
| | yl)ethyl)amino)benzoic acid | |
| 3-5 | 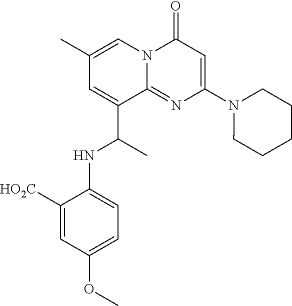 [SMILES: COc1ccc(NC(C)c2cc(C)cn3c(=O)cc(N4CCCCC4)nc23)c(C(=O)O)c1] | ¹H NMR (400 MHz, DMSO- d₆) δ = 8.50 (s, 1 H), 7.53 (d, J = 1.6 Hz, 1 H), 7.35 (d, J = 2.8 Hz, 1 H), 6.89 (dd, J = 8.8, 2.8 Hz, 1 H), 6.27 (d, J = 9.2 Hz, 1 H), 5.61 (s, 1 H), 5.15 (q, J = 6.4 Hz, 1 H), 3.65 (s, 4 H), 3.62 (s, 3 H), 2.21 (s, 3 H), 1.64 d, J = 4.4 Hz, 3 H), 1.55-1.53 (m, 6 H). LCMS [M + 1]⁺ = 437.3. |
| | 5-methoxy-2-((1-(7-methyl-4-oxo-2-(piperidin-1- | |
| | yl)-4H-pyrido[1,2-a]pyrimidin-9- | |
| | yl)ethyl)amino)benzoic acid | |
| 3-6 | 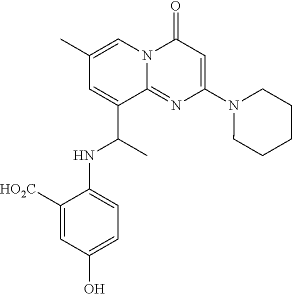 [SMILES: Cc1cc(C(C)Nc2ccc(O)cc2C(=O)O)c2nc(N3CCCCC3)cc(=O)n2c1] | ¹H NMR (400 MHz, DMSO- d₆) δ = 8.61-8.82 (m, 1 H), 8.50 (s, 1 H), 7.51 (d, J = 1.6 Hz, 1 H), 7.24 (d, J = 2.8 Hz, 1 H), 6.74 (dd, J = 8.8, 2.8 Hz, 1 H), 6.20 (d, J = 9.2 Hz, 1 H), 5.61 (s, 1 H), 5.11 (q, J = 6.4 Hz, 1 H), 3.55-3.73 (m, 4 H), 2.22 (s, 3 H), 1.64 (d, J = 4.4 Hz, 3 H), 1.54-1.52 (m, 6 H). LCMS [M + 1]⁺ = 423.2. |
| | 5-hydroxy-2-((1-(7-methyl-4-oxo-2-(piperidin-1- | |
| | yl)-4H-pyrido[1,2-a]pyrimidin-9- | |
| | yl)ethyl)amino)benzoic acid | |
| 3-7 | 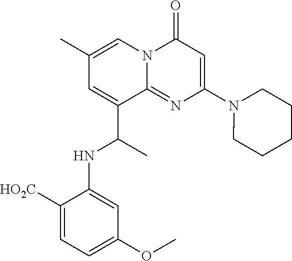 [SMILES: COc1ccc(C(=O)O)c(NC(C)c2cc(C)cn3c(=O)cc(N4CCCCC4)nc23)c1] | ¹H NMR (400 MHz, CD₃OD) δ = 8.55 (s, 1 H), 7.84 (d, J = 8.8 Hz, 1 H), 7.64 (d, J = 2.0 Hz, 1 H), 6.13 (dd, J = 8.8, 2.4 Hz, 1 H), 5.79 (d, J = 2.4 Hz, 1 H), 5.73 (s, 1H), 5.35 (q, J = 6.8 Hz, 1 H), 3.79-3.73 (m, 4 H), 3.55 (s, 3 H), 2.27 (d, J = 0.8 Hz, 3 H), 1.74-1.71 (m, 2 H), 1.67 (d, J = 4.4 Hz, 4 H), 1.61 (d, J = 6.8 Hz, 3 H). LCMS [M + 1]⁺ = 437.3. |
| | 4-methoxy-2-((1-(7-methyl-4-oxo-2-(piperidin-1- | |
| | yl)-4H-pyrido[1,2-a]pyrimidin-9- | |
| | yl)ethyl)amino)benzoic acid | |
| 3-8 | 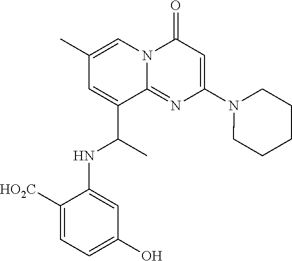 [SMILES: Cc1cc(C(C)Nc2cc(O)ccc2C(=O)O)c2nc(N3CCCCC3)cc(=O)n2c1] | ¹H NMR (400 MHz, DMSO- d₆) δ = 12.19 (s, 1H), 9.82 (s, 1H), 8.52 (s, 1H), 8.42 (br d, J = 4.8 Hz, 1H), 7.65 (d, J = 8.8 Hz, 1H), 7.49 (s, 1H), 5.98 (br d, J = 8.8 Hz, 1H), 5.64 (s, 2H), 5.08-5.06 (m, 1H), 3.65 (s, 4H), 2.23 (s, 3H), 1.63 (br d, J = 4.8 Hz, 2H), 1.55 (br d, J = 6.4 Hz, 7H). LCMS [M + 1]⁺ = 423.1. |
| | 4-hydroxy-2-((1-(7-methyl-4-oxo-2-(piperidin-1- | |
| | yl)-4H-pyrido[1,2-a]pyrimidin-9- | |
| | yl)ethyl)amino)benzoic acid | |
| 3-9 | 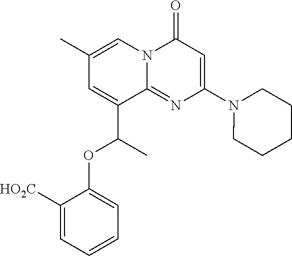 [SMILES: Cc1cc(C(C)Oc2ccccc2C(=O)O)c2nc(N3CCCCC3)cc(=O)n2c1] | ¹H NMR (400 MHz, DMSO- d₆) δ = 10.54-10.45 (s, 1H), 8.60-8.55 (s, 1H), 7.91-7.82 (m, 2H), 7.57- 7.49 (m, 1H), 7.02-6.91 (m, 2H), 6.62 (m, 1H), 5.58 (s, 1H), 3.60 (br s, 4H), 2.32 (s, 3H), 1.69 (d, J = 6.4 Hz, 3H), 1.62-1.57 (m, 2H), 1.47 (br s, 4H). LCMS [M + 1]⁺ = 408.1. |
| | 2-(1-(7-methyl-4-oxo-2-(piperidin-1-yl)-4H- | |
| | pyrido[1,2-a]pyrimidin-9-yl)ethoxy)benzoic acid | |
| 3-10 | 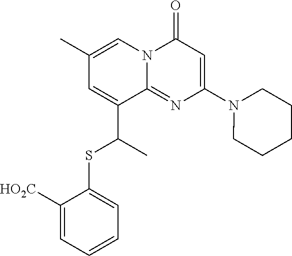 [SMILES: Cc1cc(C(C)Sc2ccccc2C(=O)O)c2nc(N3CCCCC3)cc(=O)n2c1] | ¹H NMR (400 MHz, DMSO- d₆) δ = 13.30-12.81 (s, 1H), 8.52 (s, 1H), 7.88- 7.81 (m, 2H), 7.41 (m, 2H), 7.20-7.15 (m, 1H), 5.60 (s, 1H), 5.34 (q, J = 6.8 Hz, 1H), 3.57 (br d, J = 5.2 Hz, 4H), 2.28 (s, 3H), 1.66 (br d, J = 6.8 Hz, 3H), 1.58 (br d, J = 4.4 Hz, 2H), 1.45 (br d, J = 4.8 Hz, 4H). LCMS [M + 1]⁺ = 424.3. |
| | 2-((1-(7-methyl-4-oxo-2-(piperidin-1-yl)-4H- | |
| | pyrido[1,2-a]pyrimidin-9-yl)ethyl)thio)benzoic | |
| | acid | |
| 3-11 | 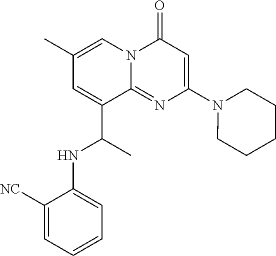 [SMILES: Cc1cc(C(C)Nc2ccccc2C#N)c2nc(N3CCCCC3)cc(=O)n2c1] | ¹H NMR (400 MHz, DMSO- d₆) δ = 8.52 (s, 1H), 7.78 (d, J = 2.0 Hz, 1H), 7.49 (dd, J = 1.2, 7.6 Hz, 1H), 7.32 (t, J = 8.0 Hz, 1H), 6.65 (t, J = 7.2 Hz, 1H), 6.48 (dd, J = 8.4, 16.4 Hz, 2H), 6.51- 6.45 (m, 2H), 5.25 (quin, J = 6.4 Hz, 1H), 3.65 (br d, J = 5.6 Hz, 4H), 2.25 (s, 3H), 1.65-1.60 (m, 2H), 1.60- 1.53 (m, 7H). LCMS [M + 1]⁺ = 388.2. |
| | 2-((1-(7-methyl-4-oxo-2-(piperidin-1-yl)-4H- | |
| | pyrido[1,2-a]pyrimidin-9- | |
| | yl)ethyl)amino)benzonitrile | |
| 3-12 | 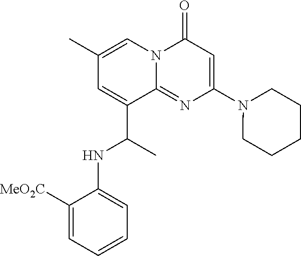 [SMILES: COC(=O)c1ccccc1NC(C)c1cc(C)cn2c(=O)cc(N3CCCCC3)nc12] | ¹H NMR (400 MHz, DMSO- d₆) δ = 8.52 (s, 1H), 8.23 (d, J = 6.4 Hz, 1H), 7.82 (dd, J = 1.6, 8.0 Hz, 1H), 7.59 (d, J = 2.0 Hz, 1H), 7.33-7.22 (m, 1H), 6.61-6.54 (m, 1H), 6.41 (d, J = 8.4 Hz, 1H), 5.63 (s, 1H), 5.24 (quin, J = 6.4 Hz, 1H), 3.85 (s, 3H), 3.66 (br s, 4H), 2.23 (d, J = 0.8 Hz, 3H), 1.68- 1.62 (m, 2H), 1.61-1.51 (m, 7H). LCMS [M + 1]⁺ = 421.3. |
| | methyl 2-((1-(7-methyl-4-oxo-2-(piperidin-1-yl)- | |
| | 4H-pyrido[1,2-a]pyrimidin-9- | |
| | yl)ethyl)amino)benzoate | |
| 3-13 | 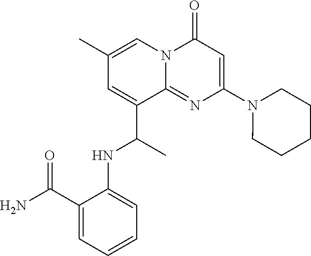 [SMILES: Cc1cc(C(C)Nc2ccccc2C(N)=O)c2nc(N3CCCCC3)cc(=O)n2c1] | ¹H NMR (400 MHz, DMSO- d₆) δ = 8.74 (d, J = 6.4 Hz, 1H), 8.51 (s, 1H), 7.91 (s, 1H), 7.62 (dd, J = 1.2, 8.0 Hz, 1H), 7.53 (d, J = 2.0 Hz, 1H), 7.25 (s, 1H), 7.18- 7.04 (m, 1H), 6.51 (t, J = 7.6 Hz, 1H), 6.27 (d, J = 8.4 Hz, 1H), 5.63 (s, 1H), 5.16 (quin, J = 6.4 Hz, 1H), 3.66 (s, 4H), 2.22 (s, 3H), 1.75- 1.49 (m, 9H). LCMS [M + 1]⁺ = 406.3. |
| | 2-((1-(7-methyl-4-oxo-2-(piperidin-1-yl)-4H- | |
| | pyrido[1,2-a]pyrimidin-9- | |
| | yl)ethyl)amino)benzamide | |
| 3-14 | 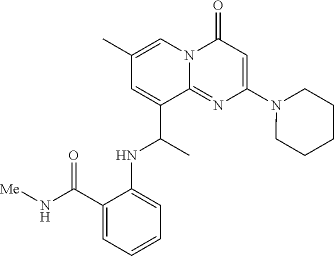 [SMILES: CNC(=O)c1ccccc1NC(C)c1cc(C)cn2c(=O)cc(N3CCCCC3)nc12] | ¹H NMR (400 MHz, DMSO- d₆) δ = 8.54-8.46 (m, 2H), 8.36 (br d, J = 4.4 Hz, 1H), 7.58-7.49 (m, 2H), 7.16- 7.04 (m, 1H), 6.53 (t, J = 7.2 Hz, 1H), 6.28 (d, J = 8.4 Hz, 1H), 5.63 (s, 1H), 5.16 (quin, J = 6.4 Hz, 1H), 3.66 (br s, 4H), 2.79 (d, J = 4.4 Hz, 3H), 2.22 (s, 3H), 1.65 (br d, J = 4.8 Hz, 2H), 1.61- 1.48 (m, 7H). LCMS [M + 1]⁺ = 420.2. |
| | N-methyl-2-((1-(7-methyl-4-oxo-2-(piperidin-1- | |
| | yl)-4H-pyrido[1,2-a]pyrimidin-9- | |
| | yl)ethyl)amino)benzamide | |
| 3-15 | 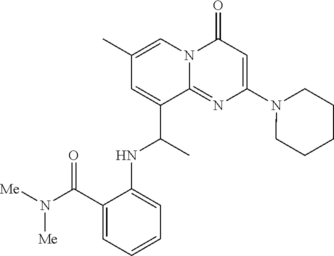 [SMILES: Cc1cc(C(C)Nc2ccccc2C(=O)N(C)C)c2nc(N3CCCCC3)cc(=O)n2c1] | ¹H NMR (400 MHz, DMSO- d₆) δ = 8.50 (s, 1H), 7.62 (d, J = 1.6 Hz, 1H), 7.32-6.95 (m, 2H), 6.59 (br t, J = 7.2 Hz, 1H), 6.41 (br d, J = 8.0 Hz, 1H), 5.87 (br d, J = 7.6 Hz, 1H), 5.62 (s, 1H), 5.16 (br d, J = 6.8 Hz, 1H), 3.66 (br s, 4H), 2.93 (br s, 6H), 2.22 (s, 3H), 1.82-1.49 (m, 9H). LCMS [M + 1]⁺ = 434.3. |
| | N,N-dimethyl-2-((1-(7-methyl-4-oxo-2-(piperidin- | |
| | 1-yl)-4H-pyrido[1,2-a]pyrimidin-9- | |
| | yl)ethyl)amino)benzamide | |
| 3-16 | 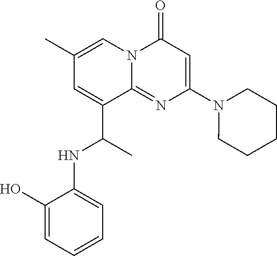 [SMILES: Cc1cc(C(C)Nc2ccccc2O)c2nc(N3CCCCC3)cc(=O)n2c1] | ¹H NMR (400 MHz, DMSO- d₆) δ = 9.34 (s, 1H), 8.49 (s, 1H), 7.64 (d, J = 2.0Hz, 1H), 6.66 (dd, J = 1.2, 7.6 Hz, 1H), 6.48 (td J = 1.2, 7.6 Hz, 1H), 6.37 (td, J = 1.2, 8.0Hz, 1H),, 6.19 (br d, J = 7.6 Hz, 1H), 5.61 (s, 1H), 5.26 (br d, J = 7.6 Hz, 1H), 5.07 (quin, J = 6.8 Hz, 1H), 3.66 (br s, 4H), 2.22 (s, 3H), 1.72-1.55 (m, 6H), 1.53 (d, J = 6.8 Hz, 3H). LCMS [M + 1]⁺ = 379.2. |
| | 9-(1-((2-hydroxyphenyl)amino)ethyl)-7-methyl-2- | |
| | (piperidin-1-yl)-4H-pyrido[1,2-a]pyrimidin-4-one | |
| 3-17 | 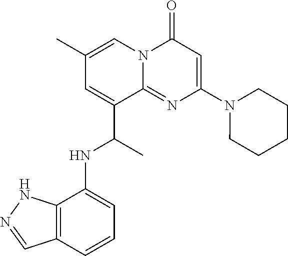 [SMILES: Cc1cc(C(C)Nc2cccc3cn[nH]c23)c2nc(N3CCCCC3)cc(=O)n2c1] | ¹H NMR (400 MHz, DMSO- d₆) δ = 12.74 (s, 1 H), 8.52 (s, 1 H), 7.94 (s, 1 H), 7.62 (s, 1 H), 6.92 (d, J = 8.0 Hz, 1 H), 6.74 (t, J = 7.6 Hz, 1 H), 6.18 (d, J = 6.0 Hz, 1 H), 5.92 (d, J = 7.2 Hz, 1 H), 5.63 (s, 1 H), 5.30 (quin, J = 6.4 Hz, 1H), 3.66 (s, 4 H), 2.20 (s, 3 H), 1.57-1.71 (m, 6 H), 1.55 (d, J = 3.2 Hz, 3 H). LCMS [M + 1]⁺ = 403.2. |
| | 9-(1-((1H-indazol-7-yl)amino)ethyl)-7-methyl-2- | |
| | (piperidin-1-yl)-4H-pyrido[1,2-a]pyrimidin-4-one | |
| 3-18 | 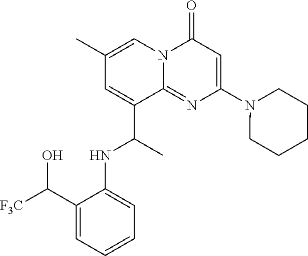 [SMILES: Cc1cc(C(C)Nc2ccccc2C(O)C(F)(F)F)c2nc(N3CCCCC3)cc(=O)n2c1] | ¹H NMR (400 MHz, DMSO- d₆) δ = 8.50 (br s, 1H), 7.65- 7.45 (m, 1H), 7.27 (br d, J = 7.2 Hz, 1H), 7.02-6.90 (m, 2H), 6.56 (t, J = 7.2 Hz, 1H), 6.20 (dd, J = 4.0, 8.0 Hz, 1H), 6.15-6.00 (m, 1H), 5.62 (s, 1H), 5.59- 5.49 (m, 1H), 5.16 (q, J = 6.8 Hz, 1H), 3.66 (br s, 4H), 2.20 (d, J = 9.6 Hz, 2H), 2.24-2.16 (m, 1H), 1.68- 1.49 (m, 9H). LCMS [M + 1]⁺ = 461.1. |
| | 7-methyl-2-(piperidin-1-yl)-9-(1-((2-(2,2,2- | |
| | trifluoro-1-hydroxyethyl)phenyl)amino)ethyl)-4H- | |
| | pyrido[1,2-a]pyrimidin-4-one | |
| 3-19 | 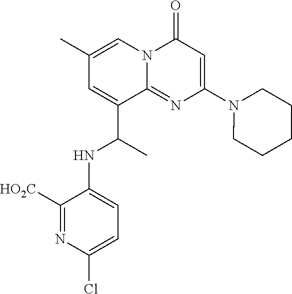 [SMILES: Cc1cc(C(C)Nc2ccc(Cl)nc2C(=O)O)c2nc(N3CCCCC3)cc(=O)n2c1] | ¹H NMR (400 MHz, DMSO- d₆) δ = 8.74-8.91 (m, 1 H), 8.51 (s, 1 H), 7.57 (d, J = 0.8 Hz, 1 H), 7.24 (d, J = 8.8 Hz, 1 H), 6.83 (d, J = 8.8 Hz, 1 H), 5.61 (s, 1 H), 5.16 (q, J = 6.4 Hz, 1 H), 3.64 (s, 4 H), 2.22 (s, 3 H), 1.68-1.58 (m, 3 H), 1.58- 1.48 (m, 6 H). LCMS [M + 1]⁺ = 442.2. |
| | 6-chloro-3-((1-(7-methyl-4-oxo-2-(piperidin-1-yl)- | |
| | 4H-pyrido[1,2-a]pyrimidin-9- | |
| | yl)ethyl)amino)picolinic acid | |
| 3-20 | 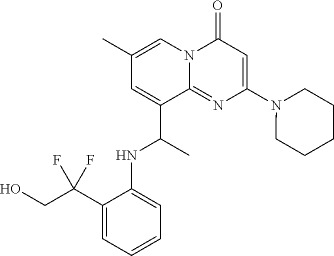 [SMILES: Cc1cc(C(C)Nc2ccccc2C(F)(F)CO)c2nc(N3CCCCC3)cc(=O)n2c1] | ¹H NMR (400 MHz, DMSO- d₆) δ = 8.52 (s, 1H), 7.66 (d, J = 2.0 Hz, 1H), 7.24 (dd, J = 1.2, 7.8 Hz, 1H), 7.13 (br t, J = 6.8 Hz, 1H), 6.64 (t, J = 7.6 Hz, 1H), 6.37 (br d, J = 8.4 Hz, 1H), 5.85 (q, J = 6.4 Hz, 1H), 5.63 (s, 1H), 5.53 (br d, J = 2.0 Hz, 1H), 5.19-5.11 (m, 1H), 3.96 (dt, J = 6.4, 14.0 Hz, 2H), 3.66 (br s, 4H), 2.24 (d, J = 0.8 Hz, 3H), 1.65 (br d, J = 4.4 Hz, 2H), 1.55 (br d, J = 6.8 Hz, 7H). LCMS [M + 1]⁺ = 443.2. |
| | 9-(1-((2-(1,1-difluoro-2- | |
| | hydroxyethyl)phenyl)amino)ethyl)-7-methyl-2- | |
| | (piperidin-1-yl)-4H-pyrido[1,2-a]pyrimidin-4-one | |
| 3-21 | 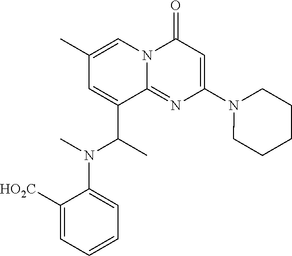 [SMILES: Cc1cc(C(C)N(C)c2ccccc2C(=O)O)c2nc(N3CCCCC3)cc(=O)n2c1] | ¹H NMR (400 MHz, CD₃OD) δ = 8.61 (s, 1H), 8.12 (d, J = 6.8 Hz, 1H), 7.86 (d, J = 1.6 Hz, 1H), 7.67-7.56 (m, 2H), 7.48-7.39 (m, 1H), 5.70-5.57 (m, 2H), 3.73 (br d, J = 5.2 Hz, 4H), 2.95 (s, 3H), 2.36 (s, 3H), 1.75 (br d, J = 4.8 Hz, 2H), 1.67 (br d, J = 4.0 Hz, 4H), 1.53 (d, J = 6.8 Hz, 3H). LCMS [M + 1]⁺ = 421.1. |
| | 2-(methyl(1-(7-methyl-4-oxo-2-(piperidin-1-yl)- | |
| | 4H-pyrido[1,2-a]pyrimidin-9- | |
| | yl)ethyl)amino)benzoic acid | |
| 3-22 | 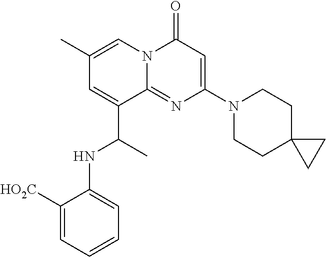 [SMILES: Cc1cc(C(C)Nc2ccccc2C(=O)O)c2nc(N3CCC4(CC3)CC4)cc(=O)n2c1] | ¹H NMR (400 MHz, DMSO- d₆) δ = 17.78-12.69 (m, 1H), 8.53 (s, 1H), 8.39 (br d, J = 4.0 Hz, 1H), 7.81 (dd, J = 1.6, 8.0 Hz, 1H), 7.57 (d, J = 2.0 Hz, 1H), 7.32-7.16 (m, 1H), 6.55 (t, J = 7.2 Hz, 1H), 6.36 (d, J = 8.4 Hz, 1H), 5.68 (s, 1H), 5.22-5.20 (m, 1H), 3.74 (s, 4H), 2.23 (s, 3H), 1.58 (d, J = 6.4 Hz, 3H), 1.40 (t, J = 5.2 Hz, 4H), 0.37 (s, 4H). LCMS [M + 1]⁺ = 433.2. |
| | 2-((1-(7-methyl-4-oxo-2-(6-azaspiro[2.5]octan-6- | |
| | yl)-4H-pyrido[1,2-a]pyrimidin-9- | |
| | yl)ethyl)amino)benzoic acid | |

### Example 4-1

### N-(1-(7-methyl-4-oxo-2-(piperidin-1-yl)-4H-pyrido[1,2-a]pyrimidin-9-yl)ethyl)acrylamide

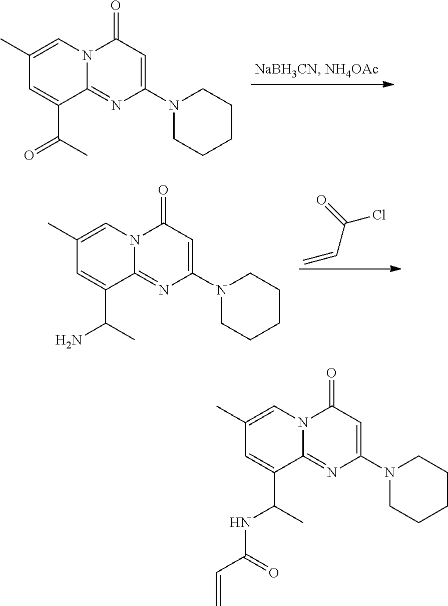

Step A: To a solution of 9-acetyl-7-methyl-2-(piperidin-1-yl)-4H-pyrido[1,2-a]pyrimidin-4-one (400 mg, 1.40 mmol, 1.00 eq.) in methanol (4.00 mL) was added ammonium acetate (1.08 g, 14.0 mmol, 10.0 eq.) and acetic acid (84.18 mg, 1.40 mmol, 80.17 μL, 1.00 eq.), and the reaction mixture was heated at 65° C. for one hour. Then sodium cyanoborohydride (106 mg, 1.68 mmol, 1.20 eq.) was added to mixture in one portion, and the reaction mixture was heated at 65° C. for one hour. Upon completion, the mixture was cooled to 25° C. and quenched with saturated ammonium chloride aqueous solution (1.00 mL) and further diluted with water (5.00 mL). The mixture was then extracted with dichloromethane (20.0 mL×2), and the combined organic phases were washed with brine (10.0 mL), dried over anhydrous sodium sulfate, filtered, and concentrated under reduced pressure to give a residue. The residue was purified by prep-HPLC (conditions: Welch Ultimate XB_C_{18 20}-35 um; 100 A, mobile phase: phase A: 0.225% formic acid in water, phase B: acetonitrile; B %: 5-100%) to give 9-(1-aminoethyl)-7-methyl-2-(piperidin-1-yl)-4H-pyrido[1,2-a]pyrimidin-4-one (80.0 mg. 279 μmol, 19.9% yield) as a green solid. LCMS [M+1]+: 287.2.

Step B: To a solution of 9-(1-aminoethyl)-7-methyl-2-(piperidin-1-yl)-4H-pyrido[1,2-a]pyrimidin-4-one (50.0 mg, 175 μmol, 1.00 eq.), triethylamine (35.3 mg, 349 μmol, 48.6 μL, 2.00 eq.) in dichloromethane (1.00 mL) was added acryloyl chloride (17.4 mg, 192 μmol, 15.7 L, 1.10 eq.) dropwise at 0° C., then the mixture was warmed to 20° C. and stirred for 30 minutes. After this time, the reaction mixture was concentrated under reduced pressure to give a residue. The residue was purified by prep-HPLC (basic condition, column: Waters Xbridge 150×25 mm×5 um; mobile phase: phase A: 0.1% ammonium hydroxide in water (v/v), phase B: acetonitrile; B %: 25%-55%) to give N-(1-(7-methyl-4-oxo-2-(piperidin-1-yl)-4H-pyrido[1,2-a]pyrimidin-9-yl)ethyl)acrylamide (5.64 mg, 16.4 μmol, 9.38% yield, 98.9% purity) as a white solid. LCMS [M+1]+: 341.0.

¹H NMR (400 MHz, DMSO-de) o=8.56 (d, J=7.6 Hz, 1H), 8.52 (s, 1H), 7.51 (d, J=2.0 Hz, 1H), 6.32 (dd, J=10.0, 17.2 Hz, 1H), 6.09 (dd, J=2.0, 17.2 Hz, 1H), 5.64-5.60 (m, 1H), 5.59 (s, 1H), 5.52 (quin, J=7.2 Hz, 1H), 3.63 (br s, 4H), 2.29 (d, J=0.8 Hz, 3H), 1.67-1.59 (m, 2H), 1.53 (br d, J=4.4 Hz, 4H), 1.42 (d, J=7.2 Hz, 3H).

¹H NMR (400 MHz, DMSO-d₆, T=80° C.) δ=8.52 (s, 1H), 8.26 (br d, J=5.6 Hz, 1H), 7.55 (s, 1H), 6.36-6.26 (m, 1H), 6.10 (dd, J=1.6, 17.2 Hz, 1H), 5.62-5.53 (m, 3H), 3.66-3.61 (m, 4H), 2.30 (s, 3H), 1.70-1.62 (m, 2H), 1.61-1.53 (m, 4H), 1.46 (d, J=6.8 Hz, 3H).

The following compound is prepared essentially according to the procedures set forth in the above schemes and examples.

| Ex. # | Structure | Spectral Data |
|---|---|---|
| 4-2 | 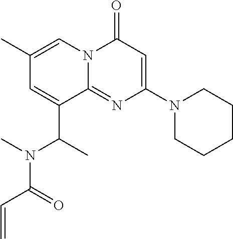 [SMILES: C=CC(=O)N(C)C(C)c1cc(C)cn2c(=O)cc(N3CCCCC3)nc12] | ¹H NMR (400 MHz, DMSO- d₆, T = 80° C.) δ = 8.56 (s, 1 H), 7.68 (s, 1 H), 6.74 (s, 1 H), 6.14 (d, J = 16.8 Hz, 2 H), 5.68-5.58 (m, 1 H), 5.50 (s, 1 H), 3.61-3.47 (m, 4 H), 2.75-2.62 (m, 3 H), 2.34 (s, 3 H), 1.65-1.52 (m, 3 H), 1.48 (d, J = 4.8 Hz, 6 H). LCMS [M + 1]⁺ = 355.2. |
| | N-methyl-N-(1-(7-methyl-4-oxo-2-(piperidin-1-yl)- | |
| | 4H-pyrido[1,2-a]pyrimidin-9-yl)ethyl)acrylamide | |
| 4-3 | 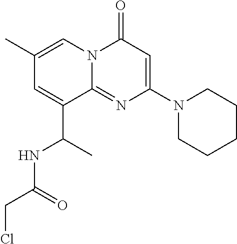 [SMILES: Cc1cc(C(C)NC(=O)CCl)c2nc(N3CCCCC3)cc(=O)n2c1] | ¹H NMR (400 MHz, DMSO- d₆) δ = 8.71 (br d, J = 7.6 Hz, 1H), 8.52 (s, 1H), 7.56 (s, 1H), 5.59 (s, 1H), 5.47- 5.38 (m, 1H), 4.13 (s, 2H), 3.63 (br s, 4H), 2.29 (s, 3H), 1.63 (br d, J = 4.8 Hz, 2H), 1.54 (br d, J = 2.8 Hz, 4H), 1.43 (d, J = 6.8 Hz, 3H). LCMS [M + 1]⁺ = 363.1. |
| | 2-chloro-N-(1-(7-methyl-4-oxo-2-(piperidin-1-yl)- | |
| | 4H-pyrido[1,2-a]pyrimidin-9-yl)ethyl)acetamide | |
| 4-4 | 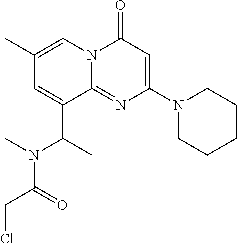 [SMILES: Cc1cc(C(C)N(C)C(=O)CCl)c2nc(N3CCCCC3)cc(=O)n2c1] | ¹H NMR (400 MHz, DMSO- d₆, T = 80° C.) δ = 8.57 (s, 1 H), 7.65 (s, 1 H), 6.04 (s, 1 H), 5.51 (s, 1 H), 4.42-4.22 (m, 2 H), 3.66-3.52 (m, 4 H), 2.75 (s, 3 H), 2.34 (s, 3 H), 1.70-1.56 (m, 3 H), 1.55-1.43 (m, 6 H). LCMS [M + 1]⁺ = 377.2. |
| | 2-chloro-N-methyl-N-(1-(7-methyl-4-oxo-2- | |
| | (piperidin-1-yl)-4H-pyrido[1,2-a]pyrimidin-9- | |
| | yl)ethyl)acetamide | |

### Example 5-1 and 5-2

### (R)-2-((1-(2-(4,4-dimethylpiperidin-1-yl)-3,7-dimethyl-4-oxo-4H-pyrido[1,2-a]pyrimidin-9-yl)ethyl)amino)benzoic acid and

### (S)-2-((1-(2-(4,4-dimethylpiperidin-1-yl)-3,7-dimethyl-4-oxo-4H-pyrido[1,2-a]pyrimidin-9-yl)ethyl)amino)benzoic acid

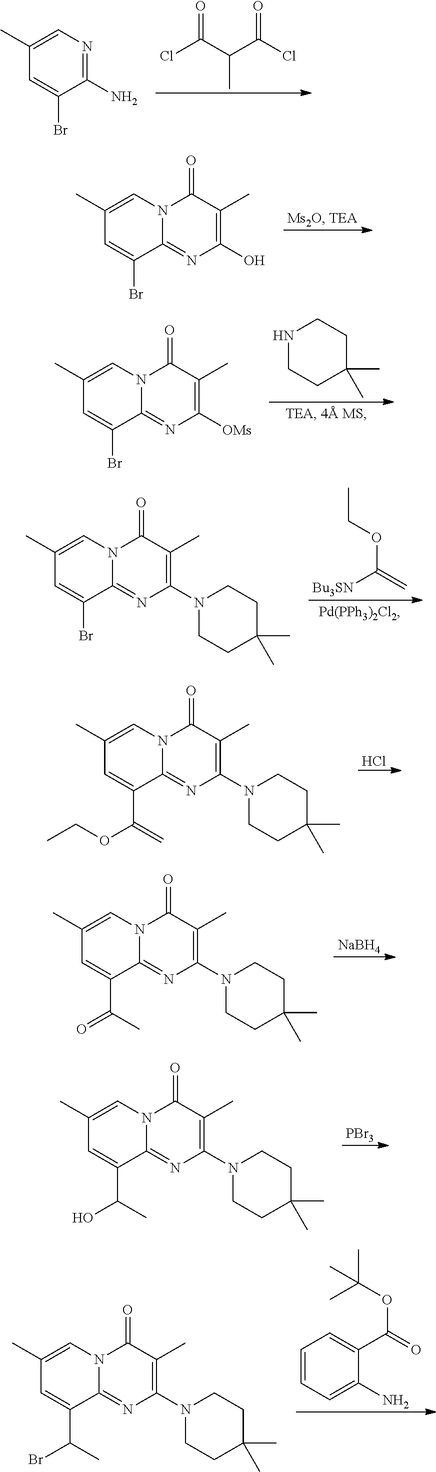

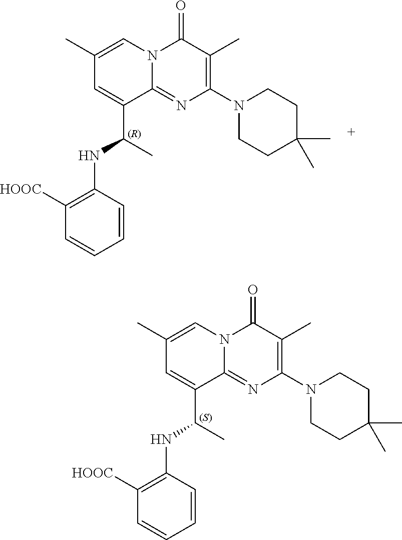

Preparation of 2-methylmalonyl dichloride: To a solution of 2-methylmalonic acid (10.0 g, 84.7 mmol, 6.85 mL, 1.00 eq.) in dichloromethane (10.0 mL) was added N,N-dimethyl formamide (309 mg, 4.23 mmol, 326 L, 0.05 eq.) and oxalyl chloride (16.1 g, 127 mmol, 11.1 mL, 1.50 eq.). The mixture was stirred at 25° C. for 2 hours. The reaction mixture was concentrated under reduced pressure to give 2-methylmalonyl dichloride (13.26 g, crude) as yellow solid.

Step A: To a solution of 2-methylmalonyl dichloride (13.3 g, 85.5 mmol, 1.00 eq.) in dichloromethane (20.0 mL) was added 3-bromo-5-methylpyridin-2-amine (16.0 g, 85.5 mmol, 1.00 eq.) at 0° C., and the mixture was stirred at 25° C. for 12 hours. The reaction mixture was then cooled to 20° C. and the resulting precipitate was filtered. The filter cake was washed with dichloromethane (50.0 mL), and the crude product was triturated with dichloromethane at 25° C. for one hour and dried under vacuum to get 9-bromo-2-hydroxy-3,7-dimethyl-4H-pyrido[1,2-a]pyrimidin-4-one (7.00 g, 25.8 mmol, 30.1% yield, 99.0% purity) as white solid. LCMS [M+3]⁺=270.8.

¹H NMR (400 MHz, DMSO-d₆) δ=8.70 (s, 1H), 8.21 (d, J=1.6 Hz, 1H), 2.34 (s, 3H), 1.96 (s, 3H).

Step B: To a solution of 9-bromo-2-hydroxy-3,7-dimethyl-4H-pyrido[1,2-a]pyrimidin-4-one (2.00 g, 7.43 mmol, 1.00 eq.) in tetrahydrofuran (20.0 mL) was added methanesulfonic anhydride (2.59 g, 14.9 mmol, 2.00 eq.) and triethylamine (2.26 g, 22.3 mmol, 3.10 mL, 3.00 eq.), and the mixture was stirred at 60° C. for 12 hours. The mixture was then treated with saturated sodium bicarbonate aqueous solution (10.0 mL), and the aqueous phase was extracted with dichloromethane (3×10 mL). The combined organic layers were dried over anhydrous sodium sulfate and concentrated under reduced pressure to give 9-bromo-3,7-dimethyl-4-oxo-4H-pyrido[1,2-a]pyrimidin-2-yl methanesulfonate (2.10 g, 5.99 mmol, 80.6% yield, 99.0% purity) as a yellow solid. LCMS [M+3]⁺=348.8.

Step C: To a solution of 9-bromo-3,7-dimethyl-4-oxo-4H-pyrido[1,2-a]pyrimidin-2-yl methanesulfonate (2.1 g, 6.05 mmol, 1.00 eq) in N,N-dimethyl formamide (15.0 mL) was added 4,4-dimethylpiperidine (1.37 g, 12.1 mmol, 2.00 eq.), 4 Å molecular sieves (5.00 mg, 6.05 mmol, 1.00 eq.) and triethylamine (1.84 g, 18.1 mmol, 2.53 mL, 3.00 eq.), and the mixture was stirred at 40° C. for 12 hours. The mixture was diluted with water (15.0 mL) and extracted with dichloromethane (15.0 mL×3). The combined organic phases were dried with anhydrous sodium sulfate, filtered, and concentrated under reduced pressure. The residue was purified by reversed phase flash column chromatography [0.1% formic acid water/acetonitrile]. The desired fractions were collected and neutralized with sodium bicarbonate solid and concentrated under vacuum. The aqueous mixture was then extracted with ethyl acetate (10.0 mL×2), and the combined organic phases were dried over anhydrous sodium sulfate, filtered, and concentrated under reduced pressure to get 9-bromo-2-(4,4-dimethylpiperidin-1-yl)-3,7-dimethyl-4H-pyrido[1,2-a]pyrimidin-4-one (0.97 g, 2.64 mmol, 43.6% yield, 99.0% purity) as a yellow solid. LCMS [M+3]⁺=365.8.

¹H NMR (400 MHz, DMSO-d₆) δ=8.61 (s, 1H), 8.13 (d, J=1.6 Hz, 1H), 3.47-3.41 (m, 4H), 2.31 (s, 3H), 2.04 (s, 3H), 1.45-1.41 (m, 4H), 0.98 (s, 6H).

Step D: A mixture of 9-bromo-2-(4,4-dimethylpiperidin-1-yl)-3,7-dimethyl-4H-pyrido[1,2-a]pyrimidin-4-one (500 mg, 1.37 mmol, 1.00 eq.), tributyl(1-ethoxyvinyl) tin (991 mg, 2.75 mmol, 927 μL, 2.00 eq.) and bis(triphenylphosphine) palladium (II) dichloride (96.3 mg, 137 μmol, 0.10 eq.) was evacuated and backfilled with nitrogen 3 times then toluene (5.00 mL) was added and the mixture was stirred at 90° C. for 12 hours under a nitrogen atmosphere. The reaction mixture was quenched with water (15.0 mL) and extracted with ethyl acetate (20.0 mL×3). The combined organic phases were dried over anhydrous sodium sulfate, filtered, and concentrated under reduced pressure to get 2-(4,4-dimethylpiperidin-1-yl)-9-(1-ethoxyvinyl)-3,7-dimethyl-4H-pyrido[1,2-a]pyrimidin-4-one (0.5 g, crude) as yellow solid.

Step E: To a solution of 2-(4,4-dimethylpiperidin-1-yl)-9-(1-ethoxyvinyl)-3,7-dimethyl-4H-pyrido[1,2-a]pyrimidin-4-one (490 mg, 1.38 mmol, 1.00 eq.) in tetrahydrofuran (5.00 mL) was added hydrochloric acid (4 M in dioxane, 1.00 mL, 2.90 eq.), and the mixture was stirred at 25° C. for one hour. The mixture was then treated with saturated aqueous saturated sodium bicarbonate (10.0 mL), and the aqueous phase was extracted with ethyl acetate (3×10.0 mL). The combined organic layers were dried over anhydrous sodium sulfate and concentrated under reduced pressure. The residue was purified by silica gel chromatography (Petroleum ether/Ethyl acetate=50/1-1/1) to give 9-acetyl-2-(4,4-dimethylpiperidin-1-yl)-3,7-dimethyl-4H-pyrido[1,2-a]pyrimidin-4-one (190 mg, 516 μmol, 37.5% yield, 89% purity) as yellow solid. LCMS [M+1]⁺=327.9.

Step F: To a solution of 9-acetyl-2-(4,4-dimethylpiperidin-1-yl)-3,7-dimethyl-4H-pyrido[1,2-a]pyrimidin-4-one (360 mg, 1.10 mmol, 1.00 eq.) in dichloromethane (3.00 mL) and ethyl alcohol (3.00 mL) was added sodium borohydride (52.2 mg, 1.38 mmol, 1.25 eq.) at −10° C., and the mixture was stirred at −10° C. for one hour. The mixture was quenched with saturated ammonium chloride aqueous solution (10.0 mL) and extracted with ethyl acetate (10.0 mL×3). The combined organic phases were dried over anhydrous sodium sulfate, filtered, and concentrated under reduced pressure to get 2-(4,4-dimethylpiperidin-1-yl)-9-(1-hydroxyethyl)-3,7-dimethyl-4H-pyrido[1,2-a]pyrimidin-4-one (320 mg, crude) as yellow solid. LCMS [M+1]⁺=330.3.

Step G: To a solution of 2-(4,4-dimethylpiperidin-1-yl)-9-(1-hydroxyethyl)-3,7-dimethyl-4H-pyrido[1,2-a]pyrimidin-4-one (100 mg. 304 μmol, 1.00 eq.) in dichloromethane (1.00 mL) was added phosphorus tribromide (164 mg, 607 μmol, 2.00 eq.) at 0° C. The mixture was stirred at 25° C. for five hours to get 9-(1-bromoethyl)-2-(4,4-dimethylpiperidin-1-yl)-3,7-dimethyl-4H-pyrido[1,2-a]pyrimidin-4-one (120 mg, crude) as yellow liquid.

Step H: To a solution of tert-butyl 2-aminobenzoate (98.5 mg, 510 μmol, 92.9 μL, 2.00 eq.) in dimethylformamide (0.50 mL) was added diisopropylethylamine (98.8 mg, 765 μmol, 133 μL, 3.00 eq.) and 9-(1-bromoethyl)-2-(4,4-dimethylpiperidin-1-yl)-3,7-dimethyl-4H-pyrido[1,2-a]pyrimidin-4-one (100 mg, 254.89 μmol, 1.00 eq.) in dichloromethane (2.00 mL) at 0° C. The pH value of the reaction was found to be pH 3-4 and reaction was stirred at 60° C. for 12 hours. Then, additional diisopropylethylamine (329 mg, 2.54 mmol, 443 μL, 10.0 eq.) was added to the reaction mixture and the pH value was adjusted to pH 8-9. The reaction was stirred at 60° C. for 3 hours. The mixture was stirred at 60° C. for 12 hours. The residue was purified by prep-HPLC (column: Waters xbridge 150×25 mm 10 um; mobile phase: phase A: 0.1% ammonium carbonate in water, phase B: acetonitrile; B %: 28%-58%). The desired fractions were collected and the aqueous layers was lyophilized to get 2-((1-(2-(4,4-dimethylpiperidin-1-yl)-3.7-dimethyl-4-oxo-4H-pyrido[1,2-a]pyrimidin-9-yl)ethyl)amino)benzoic acid (20 mg, 42.8 μmol, 16.8% yield, 96% purity) as yellow solid. LCMS [M+1]⁺=449.0.

The residue was separated by SFC (column: DAICEL CHIRALPAK IC (250 mm×30 mm, 10 um); mobile phase: phase A: acetonitrile, phase B: ammonium hydroxide in isopropyl alcohol; B %: 40%) to get the first eluting enantiomer (4.14 mg, 8.68 μmol, 19.5% yield, 94.0% purity) as yellow solid and the second eluting enantiomer (5.07 mg, 10.62 μmol, 23.8% yield, 94.0% purity) as yellow solid.

Spectral data for the first eluting enantiomer: ¹H NMR (400 MHz, CD₃OD) δ=8.60 (s, 1H), 7.91 (d, J=7.6 Hz, 1H), 7.59 (s, 1H), 7.15 (t, J=8.0 Hz, 1H), 6.54 (t, J=7.6 Hz, 1H), 6.33 (d, J=8.4 Hz, 1H), 5.41 (q, J=6.4 Hz, 1H), 3.55 (br t, J=5.6 Hz, 4H), 2.29 (s, 3H), 2.18 (s, 3H), 1.64 (d, J=6.4 Hz, 3H), 1.54 (q, J=5.2 Hz, 4H), 1.05 (s, 6H). LCMS [M+1]⁺=449.4.

Spectral data for the second eluting enantiomer: ¹H NMR (400 MHz, methanol-d₄) δ=8.60 (s, 1H), 7.91 (dd, J=1.6, 8.0 Hz, 1H), 7.60 (d, J=1.8 Hz, 1H), 7.17-7.11 (m, 1H), 6.54 (t, J=7.2 Hz, 1H), 6.32 (d, J=8.4 Hz, 1H), 5.43-5.37 (m, 1H), 3.55 (br t, J=5.6 Hz, 4H), 2.29 (s, 3H), 2.18 (s, 3H), 1.64 (d, J=6.4 Hz, 3H), 1.54 (br d, J=5.2 Hz, 4H), 1.05 (s, 6H). LCMS [M+1]⁺=449.3.

### Example 5-3

### 2-((1-(7-cyclopropyl-4-oxo-2-(piperidin-1-yl)-4H-pyrido[1,2-a]pyrimidin-9-yl)ethyl)amino)benzoic acid

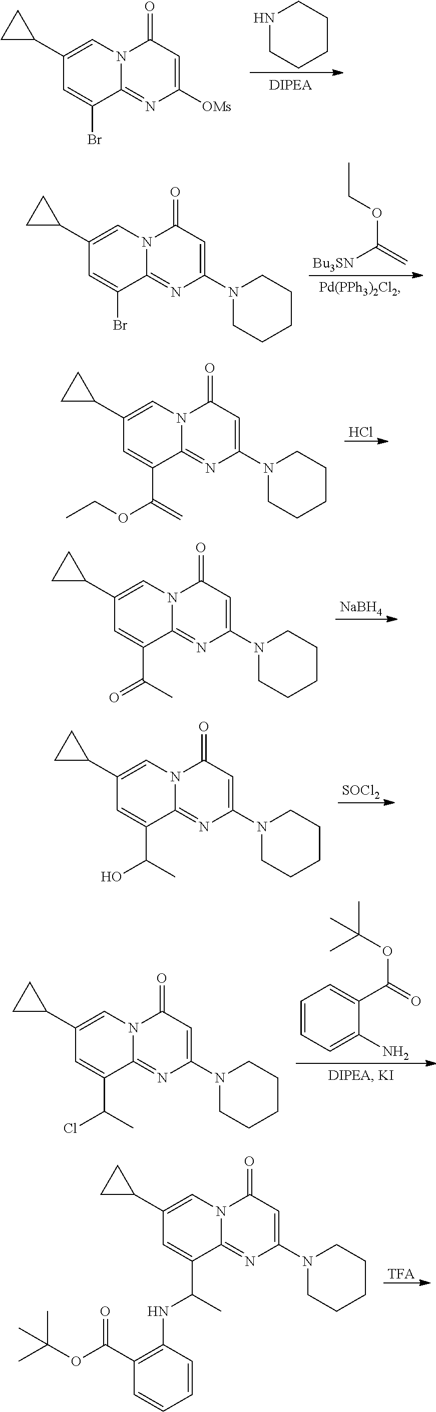

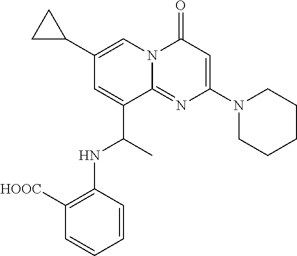 [SMILES: CC(Nc1ccccc1C(=O)O)c1cc(C2CC2)cn2c(=O)cc(N3CCCCC3)nc12]

Step A: To a solution of 9-bromo-7-cyclopropyl-4-oxo-4H-pyrido[1,2-a]pyrimidin-2-yl methanesulfonate (385 mg, 1.07 mmol, 1.00 eq.) in dimethylformamide (4.00 mL) was added diisopropylethylamine (416 mg, 3.22 mmol, 560 μL, 3.00 eq.), piperidine (183 mg, 2.14 mmol, 212 μL, 2.00 eq.) and 4 Å molecular sieves (5.00 mg, 1.07 mmol, 1.00 eq.), and the mixture was stirred at 40° C. for one hour. The mixture was quenched with water (10.0 mL) and extracted with ethyl acetate (15.0 mL×3). The combined organic phase were washed with brine (5.00 mL), dried over anhydrous sodium sulfate, filtered, and concentrated under reduced pressure to provide 9-bromo-7-cyclopropyl-2-(piperidin-1-yl)-4H-pyrido[1,2-a]pyrimidin-4-one (290 mg, 816.11 μmol, 76.14% yield, 98% purity) as a yellow solid. LCMS [M+3]⁺=350.0.

¹H NMR (400 MHz, DMSO-d₆) δ=8.55 (d, J=1.8 Hz, 1H), 7.95 (d, J=2.0 Hz, 1H), 5.59 (s, 1H), 3.66 (br s, 4H), 2.10-2.01 (m, 1H), 1.67-1.61 (m, 2H), 1.55 (m, 4H), 0.98-0.92 (m, 2H), 0.80-0.73 (m, 2H).

Step B: A mixture of 9-bromo-7-cyclopropyl-2-(piperidin-1-yl)-4H-pyrido[1,2-a]pyrimidin-4-one (290 mg, 833 μmol, 1.00 eq.), tributyl(1-ethoxyvinyl) tin (610 mg, 1.69 mmol, 570 μL, 2.03 eq.), bis(triphenylphosphine) palladium (II) dichloride (58.5 mg, 83.3 μmol, 0.10 eq.) was evacuated and backfilled with nitrogen 3 times then toluene (3.00 mL) was added and the mixture was stirred at 90° C. for 12 hours under a nitrogen atmosphere. The mixture was then diluted with water (3.00 mL) and extracted with ethyl acetate (10.0 mL×3). The combined organic phases were dried over anhydrous sodium sulfate, filtered, and concentrated under reduced pressure to get 7-cyclopropyl-9-(1-ethoxyvinyl)-2-(piperidin-1-yl)-4H-pyrido[1,2-a]pyrimidin-4-one (300 mg, crude) as yellow solid.

Step C: To a solution of 7-cyclopropyl-9-(1-ethoxyvinyl)-2-(piperidin-1-yl)-4H-pyrido[1,2-a]pyrimidin-4-one (300 mg, 884 μmol, 1.00 eq.) in tetrahydrofuran (3.00 mL) was added HCl (4 M, 0.60 mL, 2.72 eq.), and the mixture was stirred at 25° C. for one hour. The mixture was treated with saturated aqueous saturated sodium bicarbonate (5.00 mL), and the aqueous phase was extracted with ethyl acetate (3×5 mL). The combined organic phases were dried over anhydrous sodium sulfate and concentrated under reduced pressure. The residue was purified by silica gel chromatography (petroleum ether/ethyl acetate 50/1, 1/1) to give 9-acetyl-7-cyclopropyl-2-(piperidin-1-yl)-4H-pyrido[1,2-a]pyrimidin-4-one (120 mg, 382 μmol, 43.2% yield, 99% purity) as a green solid. LCMS [M+1]⁺=312.0.

Step D: To a solution of 9-acetyl-7-cyclopropyl-2-(piperidin-1-yl)-4H-pyrido[1,2-a]pyrimidin-4-one (80 mg, 257 μmol, 1.00 eq.) in dichloromethane (0.20 mL) and ethanol (0.20 mL) was added sodium borohydride (12.2 mg, 321 μmol, 1.25 eq.) at 0° C., and the mixture was stirred at 0° C. for 30 minutes. The mixture was then treated with aqueous solution of saturated ammonium chloride (5.00 mL) and extracted with ethyl acetate (5.0 mL×3). The combined organic phases were dried over anhydrous sodium sulfate, filtered, and concentrated under reduced pressure to provide 7-cyclopropyl-9-(1-hydroxyethyl)-2-(piperidin-1-yl)-4H-pyrido[1,2-a]pyrimidin-4-one (72 mg, crude) as a yellow solid. LCMS [M+1]⁺=314.2.

Step E: To a solution of 7-cyclopropyl-9-(1-hydroxyethyl)-2-(piperidin-1-yl)-4H-pyrido[1,2-a]pyrimidin-4-one (72 mg, 230 μmol, 1.00 eq.) in dichloromethane (0.50 mL) was added thionyl chloride (82.0 mg, 689 μmol, 50.0 μL, 3.00 eq.) at 0° C., and the mixture was stirred at 25° C. for 3 hours. The reaction mixture was then concentrated under reduced pressure to give crude 9-(1-chloroethyl)-7-cyclopropyl-2-(piperidin-1-yl)-4H-pyrido[1,2-a]pyrimidin-4-one (76 mg) as a yellow solid.

Step F: To a solution of tert-butyl 2-aminobenzoate (88.5 mg, 458 μmol, 83.5 μL, 2.00 eq.) and diisopropylethylamine (88.8 mg, 687 μmol, 120 μL, 3.00 eq.) in dimethylformamide (1.00 mL) was added potassium iodide (1.90 mg, 11.5 μmol, 0.05 eq.) and 9-(1-chloroethyl)-7-cyclopropyl-2-(piperidin-1-yl)-4H-pyrido[1,2-a]pyrimidin-4-one (76 mg. 229 μmol, 1.00 eq) in dimethylformamide (1.00 mL) at 0° C., and the mixture was stirred at 80° C. for 12 hours. The residue was purified by reversed phase column chromatography [water (0.1% formic acid)/acetonitrile]. The desired fractions were collected and neutralized with sodium bicarbonate solid and concentrated under reduced pressure to remove any volatiles. The aqueous phase was extracted with ethyl acetate (10.0 mL×3). The combined organic phases were dried over anhydrous sodium sulfate, filtered and, concentrated under reduced pressure to give tert-butyl 2-((1-(7-cyclopropyl-4-oxo-2-(piperidin-1-yl)-4H-pyrido[1,2-a]pyrimidin-9-yl)ethyl)amino)benzoate (45 mg, 80.1 μmol, 35.0% yield, 87% purity) as a yellow solid. LCMS [M+1]⁺=489.3.

Step G: A mixture of tert-butyl 2-((1-(7-cyclopropyl-4-oxo-2-(piperidin-1-yl)-4H-pyrido[1,2-a]pyrimidin-9-yl)ethyl)amino)benzoate (35.0 mg, 71.6 μmol, 1.00 eq.) and trifluoroacetic acid (770 mg, 6.75 mmol, 0.50 mL, 94.3 eq.) was stirred at 25° C. for 6 hours. The reaction mixture was then concentrated under reduced pressure and treated with saturated aqueous sodium bicarbonate (5.00 mL) solution. The aqueous phase was extracted with ethyl acetate (3×5.00 mL), and the combined the organic layers were dried over anhydrous sodium sulfate then concentrated under reduced pressure. The residue was purified by prep-HPLC (column: Phenomenex Luna C18 150×25 mm×10 um; mobile phase: phase A: 0.1% ammonium bicarbonate in water, phase B: acetonitrile; B %: 23%-53%). The desired fractions were collected and lyophilized to provide 2-((1-(7-cyclopropyl-4-oxo-2-(piperidin-1-yl)-4H-pyrido[1,2-a]pyrimidin-9-yl)ethyl)amino)benzoic acid (9.45 mg, 20.7 μmol, 28.9% yield, 94.8% purity) as a yellow solid. LCMS [M+1]⁺=433.2.

¹H NMR (400 MHz, CD₃OD) 0=8.49 (d, J=1.6 Hz, 1H), 7.92 (dd, J=1.6, 8.0 Hz, 1H), 7.45 (s, 1H), 7.21-7.13 (m, 1H), 6.56 (t, J=7.6 Hz, 1H), 6.30 (br d, J=7.6 Hz, 1H), 5.73 (s, 1H), 5.30 (q, J=7.2 Hz, 1H), 3.78-3.73 (m, 4H), 1.89 (m, 1H), 1.80-1.72 (m, 2H), 1.67 (br d, J=4.0 Hz, 4H), 1.62 (d, J=6.4 Hz, 3H), 0.98-0.89 (m, 2H), 0.69-0.60 (m, 1H), 0.56-0.45 (m, 1H).

### Examples 5-4 and 5-5

### (S)-2-((1-(2-(4,4-dimethylpiperidin-1-yl)-7-methyl-4-oxo-4H-pyrido[1,2-a]pyrimidin-9-yl)ethyl)amino)benzoic acid

and

### (R)-2-((1-(2-(4,4-dimethylpiperidin-1-yl)-7-methyl-4-oxo-4H-pyrido[1,2-a]pyrimidin-9-yl)ethyl)amino)benzoic acid

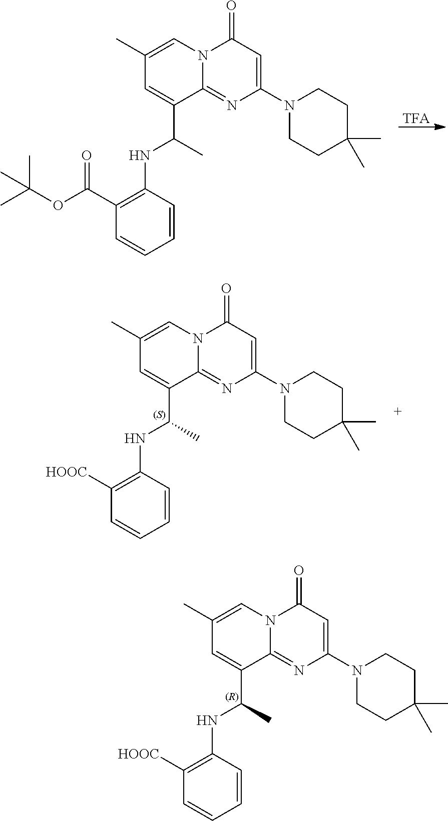

To a solution of tert-butyl 2-((1-(2-(4,4-dimethylpiperidin-1-yl)-7-methyl-4-oxo-4H-pyrido[1,2-a]pyrimidin-9-yl)ethyl)amino)benzoate (237 mg, 474 μmol, 98.0% purity, 1.00 eq.) in dichloromethane (2.00 mL) was added trifluoroacetic acid (54.0 mg, 474 μmol, 35.1 μL, 1.00 eq.), and the mixture was stirred at 25° C. for 4 hours. After this time, the mixture was concentrated into a residue by purging with nitrogen. The residue was purified by prep-HPLC (column: Phenomenex luna C18 150×25 mm×10 um; mobile phase: phase A: 0.1% formic acid in water, phase B: acetonitrile; B %: 56%-86%) to give 2-((1-(2-(4,4-dimethylpiperidin-1-yl)-7-methyl-4-oxo-4H-pyrido[1,2-a]pyrimidin-9-yl)ethyl)amino)benzoic acid (60.0 mg, 137 μmol, 28.8% yield, 98.9% purity) as a white solid. LCMS [M+1]⁺=435.2.

The enantiomers were separated using the following procedure: 60.0 mg of 2-((1-(2-(4,4-dimethylpiperidin-1-yl)-7-methyl-4-oxo-4H-pyrido[1,2-a]pyrimidin-9-yl)ethyl)amino)benzoic acid was purified by chiral prep-HPLC (column: REGIS (S,S) WHELK-O1(250 mm×25 mm, 10 um); mobile phase: phase A: acetonitrile, phase B: 0.1% ammonium hydroxide in methanol; B %: 40%-40%, 5.2 min) to give the first eluting enantiomer (22.9 mg, 51.1 μmol, 37.7% yield, 96.8% purity) as a white solid (SFC, Rt=1.246 min) and the second eluting isomer (23.9 mg, 54.4 μmol, 40.2% yield, 98.7% purity) as an slight-yellow solid (SFC, Rt=1.505 min).

Characterization for the first eluting isomer (Example 5-4): LCMS [M+1]⁺=435.2.

¹H NMR (400 MHz, DMSO-d₆) δ=8.65-8.45 (m, 2H), 7.81 (dd, J=1.2, 7.6 Hz, 1H), 7.56 (d, J=1.6 Hz, 1H), 7.20 (br t, J=7.2 Hz, 1H), 6.53 (t, J=7.2 Hz, 1H), 6.33 (br d, J=8.4 Hz, 1H), 5.64 (s, 1H), 5.21 (br d, J=6.4 Hz, 1H), 3.66 (br s, 4H), 2.23 (s, 3H), 1.57 (d, J=6.4 Hz, 3H), 1.40-1.35 (m, 4H), 0.99 (s, 6H).

Analytical SFC conditions: Column: Kromasil (S,S) Whelk-01 50×4.6 mm I.D., 3.5 um; Mobile phase: Phase A for CO₂, and Phase B: methanol+acetonitrile (0.05% DEA), 40% B in CO₂; Flow rate: 3 mL/min; Detector: PDA; Column Temp: 35° C.; Back Pressure: 100 Bar. SFC, Rt=1.246 min.

Characterization for the second eluting isomer (Example 5-5): LCMS [M+1]⁺=435.2.

¹H NMR (400 MHz, DMSO-de) δ=8.52 (s, 1H), 8.45 (br s, 1H), 7.81 (dd, J=1.2, 8.0 Hz, 1H), 7.56 (d, J=1.6 Hz, 1H), 7.22 (br t, J=7.6 Hz, 1H), 6.54 (t, J=7.6 Hz, 1H), 6.34 (br d, J=8.4 Hz, 1H), 5.64 (s, 1H), 5.28-5.13 (br d, J=4.4 Hz, 1H), 3.66 (br s, 4H), 2.23 (s, 3H), 1.57 (d, J=6.4 Hz, 3H), 1.37 (br t, J=5.6 Hz, 4H), 0.98 (s, 6H).

Analytical SFC conditions: Column: Kromasil (S,S) Whelk-01 50×4.6 mm I.D., 3.5 um. Mobile phase: Phase A for CO₂, and Phase B: methanol+acetonitrile (0.05% DEA), 40% B in CO₂; Flow rate: 3 mL/min; Detector: PDA; Column Temp: 35° C.; Back Pressure: 100 Bar. (SFC, Rₜ=1.505 min.

The following compounds are prepared essentially according to the procedures set forth in the above schemes and examples.

| Ex. # | Structure | Spectral Data |
|---|---|---|
| 5-6 | 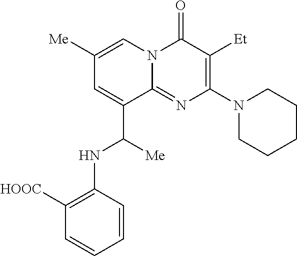 [SMILES: CCc1c(N2CCCCC2)nc2c(C(C)Nc3ccccc3C(=O)O)cc(C)cn2c1=O] | ¹H NMR (400 MHz, CD₃OD) δ ppm 8.59 (s, 1 H), 7.91 (dd, J = 8.0, 1.2 Hz, 1 H), 7.58 (d, J = 1.6 Hz, 1 H), 7.14 (t, J = 6.8 Hz, 1 H), 6.51- 6.52 (m, 1 H), 6.31 (d, J = 8.4 Hz, 1 H), 5.39 (q, J = 6.4 Hz, 1 H), 3.44- 3.55 (m, 4 H), 2.66 (q, J = 7.6 Hz, 1 H), 2.28 (s, 3 H), 1.72 (br s, 6 H), 1.63 (d, J = 6.8 Hz, 3 H), 1.29- 1.27 (s, 3 H). LCMS [M + 1]⁺ = 435.2. |
| | 2-((1-(3-ethyl-7-methyl-4-oxo-2-(piperidin- | |
| | 1-yl)-4H-pyrido[1,2-a]pyrimidin-9- | |
| | yl)ethyl)amino)benzoic acid | |
| 5-7 | 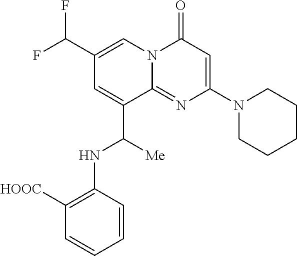 [SMILES: CC(Nc1ccccc1C(=O)O)c1cc(C(F)F)cn2c(=O)cc(N3CCCCC3)nc12] | ¹H NMR (400 MHz, CD₃OD) δ = 8.94 (d, J = 1.6 Hz, 1H), 7.95-7.89 (m, 1H), 7.75 (d, J = 1.6 Hz, 1H), 7.21-7.15 (m, 1H), 6.8 (t, J = 55.2 Hz, 1H), 6.57 (t, J = 7.6 Hz, 1H), 6.32 (d, J = 8.4 Hz, 1H), 5.75 (s, 1H), 5.30 (q, J = 6.4 Hz, 1H), 3.78 (br s, 4H), 1.81-1.72 (m, 2H), 1.71-1.64 (m, 7H). LCMS [M + 1]⁺ = 443.3. |
| | 2-((1-(7-(difluoromethyl)-4-oxo-2- | |
| | (piperidin-1-yl)-4H-pyrido[1,2-a]pyrimidin- | |
| | 9-yl)ethyl)amino)benzoic acid | |
| 5-8 | 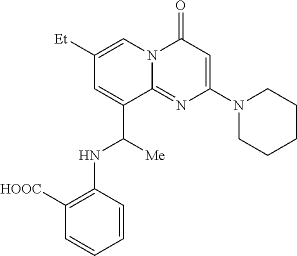 [SMILES: CCc1cc(C(C)Nc2ccccc2C(=O)O)c2nc(N3CCCCC3)cc(=O)n2c1] | ¹H NMR (400 MHz, DMSO-d₆) δ = 12.30- 13.05 (m, 1 H), 8.51 (d, J = 2.0 Hz, 1 H), 8.41 (br d, J = 5.6 Hz, 1 H), 7.81 (dd, J = 8.0, 1.6 Hz, 1 H), 7.60 (d, J = 2.0 Hz, 1 H), 7.14-7.28 (m, 1 H), 6.54 (t, J = 7.2 Hz, 1 H), 6.38 (d, J = 8.4 Hz, 1 H), 5.63 (s, 1 H), 5.22 (br t, J = 6.4 Hz, 1 H), 3.66 (br d, J = 3.6 Hz, 4 H), 2.57 (q, J = 7.2 Hz, 2 H), 1.62- 1.69 (m, 2 H), 1.50- 1.62 (m, 7 H), 1.10 (t, J = 7.6 Hz, 3 H). LCMS [M + 1]⁺ = 421.1. |
| | 2-((1-(7-ethyl-4-oxo-2-(piperidin-1-yl)-4H- | |
| | pyrido[1,2-a]pyrimidin-9- | |
| | yl)ethyl)amino)benzoic acid | |
| 5-9 | 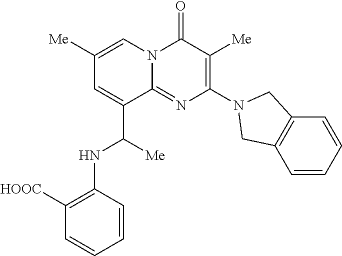 [SMILES: Cc1cc(C(C)Nc2ccccc2C(=O)O)c2nc(N3Cc4ccccc4C3)c(C)c(=O)n2c1] | ¹H NMR (400 MHz, CD₃OD) δ = 8.59 (s, 1H), 7.90 (d, J = 9.2 Hz, 1H), 7.63 (d, J = 1.6 Hz, 1H), 7.40-7.34 (m, 2H), 7.32- 7.28 (m, 2H), 7.09 (t, J = 7.2 Hz, 1H), 6.52 (t, J = 7.2 Hz, 1H), 6.32 (d, J = 8.4 Hz, 1H), 5.43 (q, J = 6.4 Hz, 1H), 5.25 (br d, J = 5.2 Hz, 4H), 2.52 (s, 3H), 2.28 (s, 3H), 1.68 (d, J = 6.8 Hz, 3H). LCMS [M +1]⁺ = 455.2. |
| | 2-((1-(2-(isoindolin-2-yl)-3,7-dimethyl-4- | |
| | oxo-4H-pyrido[1,2-a]pyrimidin-9- | |
| | yl)ethyl)amino)benzoic acid | |
| 5-9-1 and 5-9-2 | 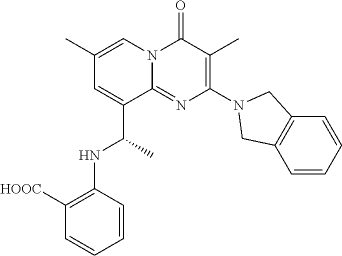 [SMILES: Cc1cc([C@H](C)Nc2ccccc2C(=O)O)c2nc(N3Cc4ccccc4C3)c(C)c(=O)n2c1] | 5-9-1 Data (first eluting isomer): ¹H NMR (400 MHz, DMSO-d₆) δ = 8.56 (s, 2H), 7.80 (dd, J = 1.4, 8.0 Hz, 1H), 7.55 (d, J = 1.6 Hz, 1H), 7.42-7.37 (m, 2H), 7.34-7.27 (m, 2H), 7.22-7.13 (m, 1H), 6.52 (t, J = 7.6 Hz, 1H), 6.37 (br d, J = 9.2 Hz, 1H), 5.37-5.30 (m, 1H), 5.23-5.13 (m, 4H), 2.42 (s, 3H), 2.25 (s, 3H), 1.63 (d, J = 6.8 Hz, 3H). LCMS [M + 1]⁺ = 455.3. |
| | (S)-2-((1-(2-(isoindolin-2-yl)-3,7-dimethyl- | |
| | 4-oxo-4H-pyrido[1,2-a]pyrimidin-9- | |
| | yl)ethyl)amino)benzoic acid | |
| | or | |
| | 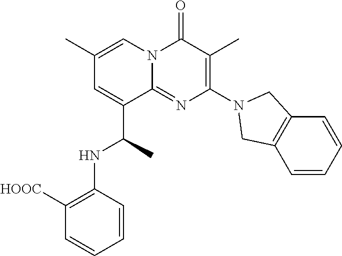 [SMILES: Cc1cc([C@@H](C)Nc2ccccc2C(=O)O)c2nc(N3Cc4ccccc4C3)c(C)c(=O)n2c1] | 5-9-2 Data (second eluting isomer): ¹H NMR (400 MHz, DMSO-d₆) δ = 8.56 (s, 2H), 7.80 (dd, J = 1.6, 8.0 Hz, 1H), 7.55 (d, J = 2.0 Hz, 1H), 7.41-7.36 (m, 2H), 7.33- 7.28 (m, 2H), 7.19 (br t, J = 7.2 Hz, 1H), 6.55- 6.49 (m, 1H), 6.38 (br d, J = 8.4 Hz, 1H), 5.39- 5.29 (m, 1H), 5.18 (s, 4H), 2.42 (s, 3H), 2.25 (s, 3H), 1.63 (d, J = 6.4 Hz, 3H). LCMS [M + 1]⁺ = 455.1. |
| | (R)-2-((1-(2-(isoindolin-2-yl)-3,7-dimethyl- | |
| | 4-oxo-4H-pyrido[1,2-a]pyrimidin-9- | |
| | yl)ethyl)amino)benzoic acid | |
| 5-10 | 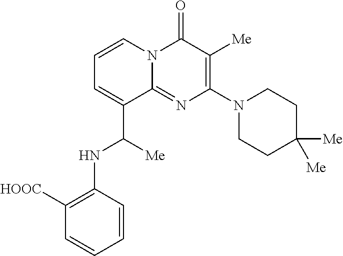 [SMILES: Cc1c(N2CCC(C)(C)CC2)nc2c(C(C)Nc3ccccc3C(=O)O)cccn2c1=O] | LCMS [M + 1]⁺ = 435.1 |
| | 2-((1-(2-(4,4-dimethylpiperidin-1-yl)-3- | |
| | methyl-4-oxo-4H-pyrido[1,2-a]pyrimidin-9- | |
| | yl)ethyl)amino)benzoic acid | |
| 5-11 | 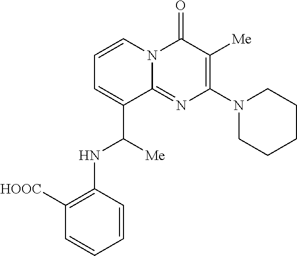 [SMILES: Cc1c(N2CCCCC2)nc2c(C(C)Nc3ccccc3C(=O)O)cccn2c1=O] | LCMS [M + 1]⁺ = 407.2 |
| | 2-((1-(3-methyl-4-oxo-2-(piperidin-1-yl)- | |
| | 4H-pyrido[1,2-a]pyrimidin-9- | |
| | yl)ethyl)amino)benzoic acid | |

### Example 6-1

### 2-((1-(2,7-dimethyl-4-oxo-4H-pyrido[1,2-a]pyrimidin-9-yl)ethyl)amino)benzoic acid

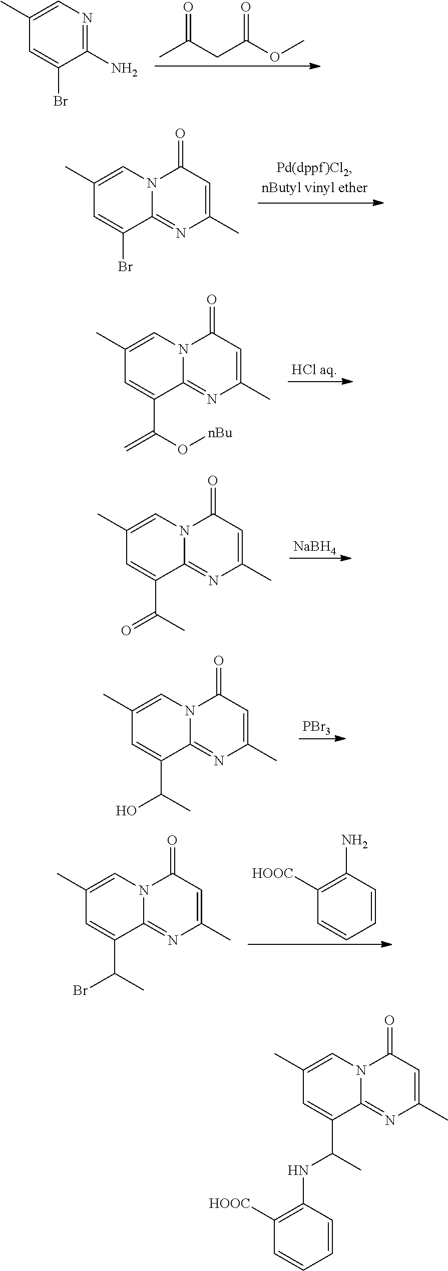

Step A: A mixture of 3-bromo-5-methyl-pyridin-2-amine (2.42 g, 12.9 mmol, 0.500 eq.), methyl 3-oxobutanoate (3.00 g, 25.8 mmol, 2.78 mL, 1.00 eq.) and bismuth (III) chloride (204 mg, 646 μmol, 42.9 μL, 0.03 eq.) was stirred at 120° C. for 12 hours. After completion of the reaction, the reaction mixture was cooled to 25° C., ethanol (3.00 mL) was added to the solution and a brown precipitate was formed. It was collected by filtration and air dried under vacuum to give 9-bromo-2,7-dimethyl-4H-pyrido[1,2-a]pyrimidin-4-one (1.85 g, 7.31 mmol, 28.3% yield) as a brown solid. LCMS [M+3]⁺=254.9.

¹H NMR (400 MHz, DMSO-d₆) δ=8.77 (s, 1H), 8.31 (d, J=1.6 Hz, 1H), 6.34 (s, 1 H), 2.39 (s, 3H), 2.37 (s, 3H).

Step B: A mixture of 9-bromo-2,7-dimethyl-4H-pyrido[1,2-a]pyrimidin-4-one (400 mg, 1.58 mmol, 1.00 eq.), n-butyl vinyl ether (475 mg, 4.74 mmol, 610 μL, 3.00 eq.), diisopropylethylamine (613 mg, 4.74 mmol, 826 μL, 3.00 eq.) and [1,1′-bis(diphenylphosphino) ferrocene]dichloropalladium (II), dichloromethane complex (129 mg, 158 μmol, 0.100 eq.) was evacuated and backfilled with nitrogen 3 times. Then n-butanol (5.00 mL) was added and the mixture was stirred at 120° C. for 12 hours under a nitrogen atmosphere. After completion of the reaction, the mixture was cooled to 25° C. to give crude 9-(1-ethoxyvinyl)-2,7-dimethyl-4H-pyrido[1,2-a]pyrimidin-4-one (380 mg, 1.56 mmol, 98.4% yield) as a brown oil which was used directly.

Step C: A mixture of 9-(1-ethoxyvinyl)-2,7-dimethyl-4H-pyrido[1,2-a]pyrimidin-4-one (380 mg, 1.56 mmol, 1.00 eq.) in hydrochloric acid (3 M in water, 2.59 mL, 5.00 eq.) was stirred at 25° C. for one hour. The mixture was then diluted with water (30.0 mL) and extracted with dichloromethane (30.0 mL×3). The combined organic phases were washed with water (30.0 mL), dried over anhydrous sodium sulfate, filtered, and concentrated under reduced pressure to give a residue. The residue was purified by column chromatography (SiO2, petroleum ether/ethyl acetate=1/0 to 3/1) to give 9-acetyl-2,7-dimethyl-4H-pyrido[1,2-a]pyrimidin-4-one (180 mg, 821 μmol, 52.8% yield, 98.6% purity) as a yellow solid.

LCMS [M+1]⁺=217.1.

Step D: To a solution of 9-acetyl-2,7-dimethyl-4H-pyrido[1,2-a]pyrimidin-4-one (180 mg. 832 μmol, 1.00 eq.) in mixture solvent of dichloromethane (1.00 mL) and methanol (1.00 mL) was added sodium borohydride (63.0 mg, 1.66 mmol, 2.00 eq.) at −20° C. under a nitrogen atmosphere, and the mixture was stirred at −20° C. for one hour. After completion of the reaction, the reaction mixture was quenched by added saturated ammonium chloride aqueous solution (20.0 mL) dropwise at 0° C. The resulting solution was then diluted with water (30.0 mL) and extracted with dichloromethane (30.0 mL×3). The combined organic phases were washed with brine (30.0 mL), dried over anhydrous sodium sulfate, filtered, and concentrated under reduced pressure to give a residue. The residue was purified by column chromatography (SiO₂, petroleum ether/ethyl acetate=0 to 3/1) to 9-(1-hydroxyethyl)-2,7-dimethyl-4H-pyrido[1,2-a]pyrimidin-4-one (130 mg, 596 μmol, 71.6% yield) as a white solid. LCMS [M+H]+=219.2.

Step E: To a solution of 9-(1-hydroxyethyl)-2,7-dimethyl-4H-pyrido[1,2-a]pyrimidin-4-one (130 mg, 596 μmol, 1.00 eq.) in dichloromethane (1.50 mL) was added phosphorus tribromide (242 mg, 893 μmol, 1.50 eq.) at 25° C., then the mixture was stirred at 40° C. for 3 hours. The mixture was then cooled to 25° C. and was adjusted to pH=8 with saturated sodium bicarbonate aqueous solution. The solution was extracted with dichloromethane (10.0 mL×3), and the combined organic phases were washed with brine (5.00 mL×3), dried over anhydrous sodium sulfate, filtered, and concentrated under reduced pressure to give 9-(1-bromoethyl)-2,7-dimethyl-4H-pyrido[1,2-a]pyrimidin-4-one (160 mg, 569 μmol, 95.5% yield) as a white solid which was used directly in the next step.

Step F: To a solution of 9-(1-bromoethyl)-2,7-dimethyl-4H-pyrido[1,2-a]pyrimidin-4-one (160 mg, 569 μmol, 1.00 eq.) in dimethylformamide (2.00 mL) was added 2-aminobenzoic acid (370 mg, 2.70 mmol, 4.74 eq.) at 25° C., then the mixture was stirred at 80° C. for one hour. The mixture was then cooled to 25° C., diluted with ethyl acetate (60.0 mL), and the organic solution was washed with brine (20.0 mL×3), dried over sodium sulfate, and concentrated under reduced pressure to give a residue. The residue was purified by prep-HPLC (column: Waters X bridge 150×25 mm×5 um; mobile phase: phase A: 0.1% ammonium hydroxide in water, phase B: acetonitrile; B %: 1%-31%) to give 2-((1-(2,7-dimethyl-4-oxo-4H-pyrido[1,2-a]pyrimidin-9-yl)ethyl)amino)benzoic acid (30.8 mg, 88.1 μmol, 15.5% yield, 96.4% purity) as a white solid. LCMS [M+1]⁺=338.1.

¹H NMR (400 MHz, DMSO-d₆) δ=9.08-8.77 (m, 1H), 8.69 (s, 1H), 7.81 (dd, J=8.0, 1.6 Hz, 1H), 7.71 (d, J=2.0 Hz, 1H), 7.16-7.08 (m, 1H), 6.49 (t, J=7.2 Hz, 1H), 6.36-6.26 (m, 2H), 5.35 (q, J=6.4 Hz, 1H), 2.44 (s, 3H), 2.30 (s, 3H), 1.57 (d, J=6.8 Hz, 3H).

The following compound is prepared essentially according to the procedures set forth in the above schemes and examples.

| Ex. # | Structure | Spectral Data |
|---|---|---|
| 6-2 | 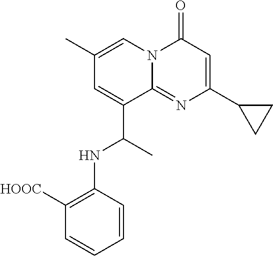 [SMILES: Cc1cc(C(C)Nc2ccccc2C(=O)O)c2nc(C3CC3)cc(=O)n2c1] | ¹H NMR (400 MHz, DMSO- d₆) δ = 9.05-8.77 (m, 1 H), 8.65 (s, 1 H), 7.81 (dd, J = 7.6, 1.6 Hz, 1 H), 7.66 (d, J = 2.0 Hz, 1 H), 7.14-7.06 (m, 1 H), 6.53-6.44 (m, 1 H), 6.40 (s, 1 H), 6.22 (d, J = 8.4 Hz, 1 H), 5.25 (q, J = 6.4 Hz, 1 H), 2.28 (d, J = 0.8 Hz, 3 H), 2.10-2.02 (m, 1 H), 1.53 (d, J = 6.4 Hz, 3 H), 1.19-1.10 (m, 2 H), 1.03 (dd, J = 7.2, 4.4 Hz, 2 H). LCMS [M + 1]⁺ = 364.1. |
| | 2-((1-(2-cyclopropyl-7-methyl-4-oxo-4H- | |
| | pyrido[1,2-a]pyrimidin-9-yl)ethyl)amino)benzoic | |
| | acid | |

### Example 7-1

### 2-((1-(7-methyl-4-oxo-2-phenyl-4H-pyrido[1,2-a]pyrimidin-9-yl)ethyl)amino)benzoic acid

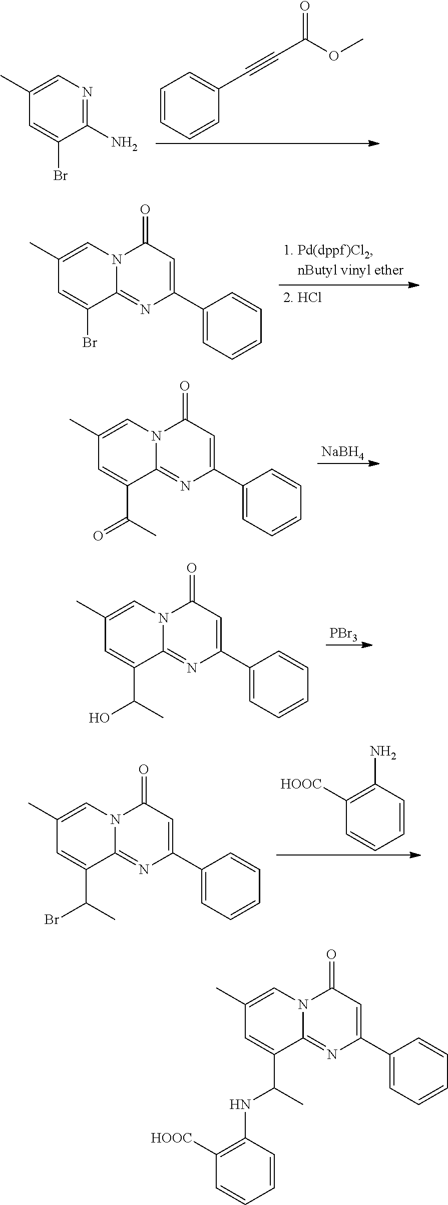

Step A: To a solution of 3-bromo-5-methylpyridin-2-amine (1.00 g, 5.35 mmol, 1.00 eq.) in glycol (10.00 mL) was added methyl 3-phenylpropiolate (1.28 g, 8.02 mmol, 1.50 eq.) at 25° C., then the mixture was stirred at 100° C. for 5 hours. The mixture was then cooled to 25° C., and ethyl acetate (100 mL) and water (100 mL) were added to the mixture. The organic phase was collected, then the aqueous phase was further extracted with ethyl acetate (100 mL×2). The combined organic phases were washed with brine (100 mL), dried over sodium sulfate, filtered, and concentrated under reduced pressure to give a residue. The residue was purified by column chromatography (SiO₂, petroleum ether/ethyl acetate=I/O to 3/1) to give 9-bromo-7-methyl-2-phenyl-4H-pyrido[1,2-a]pyrimidin-4-one (1.20 g, 3.74 mmol, 70.0% yield, 98.3% purity) as a yellow solid. LCMS [M+1]⁺=315.0.

¹H NMR (400 MHz, DMSO-d₆) δ=8.13 (d, J=1.6 Hz, 1H), 7.65-7.58 (m, 5H), 7.51 (s, 1H), 6.27 (s, 1H), 2.09 (s, 3H).

Step B: A mixture of 9-bromo-7-methyl-2-phenyl-4H-pyrido[1,2-a]pyrimidin-4-one (830 mg, 2.63 mmol, 1.00 eq.), 1-vinyloxybutane (791 mg, 7.90 mmol, 1.02 mL, 3.00 eq.), N, N-diisopropylethylamine (1.02 g, 7.90 mmol, 1.38 mL, 3.00 eq.). [1,1′-Bis(diphenylphosphino) ferrocene]dichloropalladium (II) (193 mg, 263 μmol. 0.10 eq.) was evacuated and backfilled with nitrogen 3 times. Then n-butanol (1.00 mL) was added and the mixture was stirred at 120° C. for 12 hours under a nitrogen atmosphere. After completion of the reaction, the mixture was cooled to 25° C. and HCl (3 M in water, 1.00 mL) was added, then the mixture was stirred at 25° C. for one hour. The mixture was further diluted with water (30.0 mL), then extracted with dichloromethane (30.0 mL×3). The combined organic phases were washed with water (20.0 mL), dried over anhydrous sodium sulfate, filtered, and concentrated under reduced pressure to give a residue. The residue was purified by column chromatography (SiO₂, petroleum ether/ethyl acetate=1/0 to 3/1) to give 9-acetyl-7-methyl-2-phenyl-4H-pyrido[1,2-a]pyrimidin-4-one (135 mg, 485 μmol, 18.4% yield) as a yellow solid. LCMS [M+1]⁺=279.1.

Step C: To a solution of 9-acetyl-7-methyl-2-phenyl-4H-pyrido[1,2-a]pyrimidin-4-one (130 mg, 467 μmol, 1.00 eq.) in mixture of dichloromethane (1.00 mL) and methanol (1.00 mL) was added sodium borohydride (87.2 mg, 934 μmol, 2.00 eq.) at −20° C. under a nitrogen atmosphere, and the mixture was stirred at −20° C. for one hour. After this time, a solution of ammonium chloride (saturated in water, 50.0 mL) was added dropwise to the mixture at 0° C. The resulting mixture was diluted with water (30.0 mL) and extracted with dichloromethane (30.0 mL×3). The combined organic layers were washed with brine (20.0 mL), dried over anhydrous sodium sulfate, filtered, and concentrated under reduced pressure to give a residue. The residue was purified by column chromatography (SiO₂, petroleum ether/ethyl acetate=1/0 to 3/1) to give 9-(1-hydroxyethyl)-7-methyl-2-phenyl-4H-pyrido[1,2-a]pyrimidin-4-one (90.0 mg, 321 μmol, 68.7% yield) as a yellow solid. LCMS [M+1]⁺=281.1.

Step D: To a solution of 9-(1-hydroxyethyl)-7-methyl-2-phenyl-4H-pyrido[1,2-a]pyrimidin-4-one (90.0 mg, 321 μmol, 1.00 eq.) in dichloromethane (1.00 mL) was added phosphorus tribromide (130 mg, 482 μmol, 1.50 eq.) at 25° C., the mixture was then stirred warmed to 40° C. and stirred for 3 hours at 40° C. After this time, the pH of the mixture was adjusted to to pH=8 with saturated sodium bicarbonate aqueous solution, then extracted with dichloromethane (15.0 mL×3). The combined organic phases were washed with brine (5.00 mL×3), dried over anhydrous sodium sulfate, filtered, and concentrated under reduced pressure to give 9-(1-bromoethyl)-7-methyl-2-phenyl-4H-pyrido[1,2-a]pyrimidin-4-one (110 mg, 321 μmol, 99.8% yield) as an yellow solid.

Step E: A solution of 9-(1-bromoethyl)-7-methyl-2-phenyl-4H-pyrido[1,2-a]pyrimidin-4-one (110 mg, 321 μmol, 1.00 eq.) and 2-aminobenzoic acid (87.0 mg, 634 μmol, 1.98 eq.) in dimethylformamide (1.00 mL) was a stirred at 70° C. for one hour. After this time, the mixture was cooled to 25° C., diluted with ethyl acetate (60.0 mL), and washed with brine (30.0 mL). The combined organic phases were dried over sodium sulfate and concentrated under reduced pressure to give a residue. The residue was purified by prep-HPLC (column: Waters X bridge 150×25 mm×5 um; mobile phase: phase A: 0.1% ammonium hydroxide in water, phase B: acetonitrile; B %: 9%-39%) to give 2-((1-(7-methyl-4-oxo-2-phenyl-4H-pyrido[1,2-a]pyrimidin-9-yl)ethyl)amino)benzoic acid (17.6 mg, 43.7 μmol, 13.6% yield, 99.4% purity) as a yellow solid. LCMS [M+1]⁺=400.3.

¹H NMR (400 MHz, DMSO-de) δ=8.75 (s, 1H), 8.72-8.61 (m, 1H), 8.32-8.24 (m, 2H), 7.83 (dd, J=8.0, 1.6 Hz, 1H), 7.76 (d, J=2.0 Hz, 1H), 7.59-7.49 (m, 3H), 7.19-7.11 (m, 1H), 7.04 (s, 1H), 6.52 (t, J=7.6 Hz, 1H), 6.38 (d, J=8.4 Hz, 1H), 5.55 (q, J=6.4 Hz, 1H), 2.34 (s, 3H), 1.67 (d, J=6.4 Hz, 3H).

The following compound is prepared essentially according to the procedures set forth in the above schemes and examples.

| | | Spectral |
|---|---|---|
| Ex. # | Structure | Data |
| 7-2 | 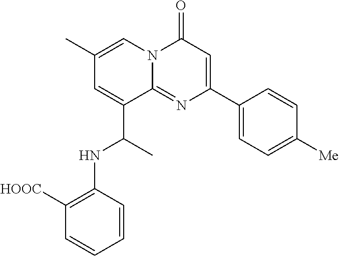 [SMILES: Cc1ccc(-c2cc(=O)n3cc(C)cc(C(C)Nc4ccccc4C(=O)O)c3n2)cc1] | |
| | 2-((1-(7-methyl-4-oxo-2-(p-tolyl)-4H-pyrido[1,2- | |
| | a]pyrimidin-9-yl)ethyl)amino)benzoic acid | |

### Example 8-1

### 2-((1-(3-cyano-7-methyl-4-oxo-2-(piperidin-1-yl)-4H-pyrido[1,2-a]pyrimidin-9-yl)ethyl)amino)benzoic acid

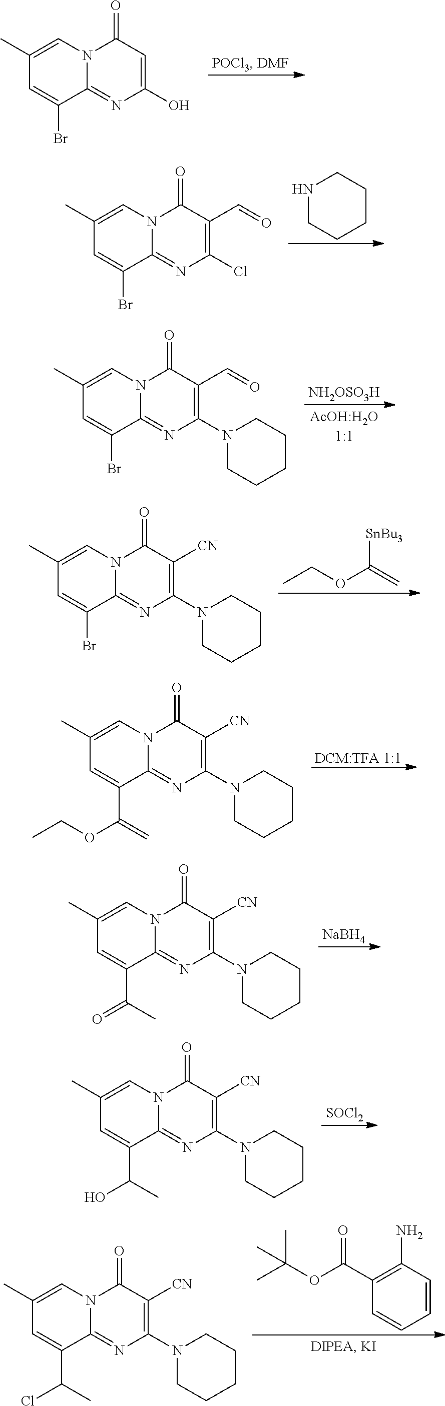

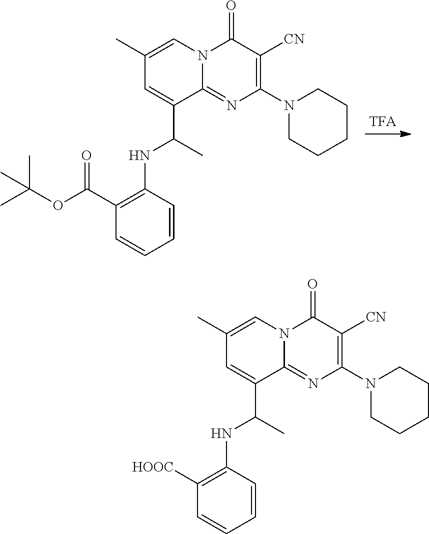 [SMILES: Cc1cc(C(C)Nc2ccccc2C(=O)OC(C)(C)C)c2nc(N3CCCCC3)c(C#N)c(=O)n2c1 ; Cc1cc(C(C)Nc2ccccc2C(=O)O)c2nc(N3CCCCC3)c(C#N)c(=O)n2c1]

Step A: To a solution of 9-bromo-2-hydroxy-7-methyl-4H-pyrido[1,2-a]pyrimidin-4-one (10.0 g, 39.2 mmol, 1.00 eq.) in dimethylformamide (100 mL) was added phosphorus oxychloride (18.0 g, 117 mmol, 10.9 mL, 3.00 eq.) at 0° C. The mixture was heat to 80° C. and stirred at 80° C. for 5 hours. The mixture was cooled and concentrated under vacuum to a residue. The residue was diluted water (50.0 mL) and extracted dichloromethane (50.0 mL×3). The combined organic layers were washed with brine (100 mL), dried over sodium sulfate, filtered and concentrated under reduced pressure to give a residue. The residue was triturated with dichloromethane (15.00 mL) to give 9-bromo-2-chloro-7-methyl-4-oxo-4H-pyrido[1,2-a]pyrimidine-3-carbaldehyde (2.00 g, 3.65 mmol, 4.65% yield, 54.8% purity) as a brown solid. LCMS [M+1]⁺=302.9.

Step B: To a solution of 9-bromo-2-chloro-7-methyl-4-oxo-4H-pyrido[1,2-a]pyrimidine-3-carbaldehyde (2.00 g, 6.63 mmol, 1.00 eq.) in tetrahydrofuran (20.0 mL) was added piperidine (1.69 g, 19.9 mmol, 1.97 mL, 3.00 eq.) and diisopropylethylamine (857 mg, 6.63 mmol, 1.16 mL, 1.00 eq.). The mixture was stirred at 70° C. for 3 hours. The mixture was diluted water (30.0 mL) and extracted with ethyl acetate (50.0 mL×3). Combined organic layers were dried over anhydrous sodium sulfate, filtered and concentrated in vacuum to give a residue. The residue was purified by silica gel chromatography (petroleum ether/ethyl acetate 50/1, 3/1) to give 9-bromo-7-methyl-4-oxo-2-(piperidin-1-yl)-4H-pyrido[1,2-a]pyrimidine-3-carbaldehyde (1.00 g, 2.68 mmol, 40.5% yield, 94.0% purity) as a yellow solid. LCMS [M+1]⁺=352.1.

Step C: To a solution of 9-bromo-7-methyl-4-oxo-2-(piperidin-1-yl)-4H-pyrido[1,2-a]pyrimidine-3-carbaldehyde (2.00 g, 5.71 mmol, 1.00 eq.) in acetic acid (2.00 mL) and water (8.00 mL) was added hydroxylamine-O-sulfonic acid (969 mg, 8.57 mmol, 1.50 eq.) at 0° C. The mixture heat to 60° C. and stirred at 60° C. for 12 hours. The mixture was filtered to get a residue. The residue was triturated with ethyl acetate (3.00 mL) to give 9-bromo-7-methyl-4-oxo-2-(piperidin-1-yl)-4H-pyrido[1,2-a]pyrimidine-3-carbonitrile (1.00 g, 2.76 mmol, 48.4% yield, 90.0% purity) as a yellow solid. LCMS [M+1]⁺=349.1.

Step D: A mixture of 9-bromo-7-methyl-4-oxo-2-(piperidin-1-yl)-4H-pyrido[1,2-a]pyrimidine-3-carbonitrile (1.00 g, 2.88 mmol, 1.00 eq.), tributyl(1-ethoxyvinyl) stannane (2.08 g, 5.76 mmol, 1.94 mL, 2.00 eq.) and bis(triphenylphosphine) palladium (II) dichloride (202 mg, 288 μmol, 0.100 eq.) was evacuated and backfilled with nitrogen for 3 times. Then toluene (10.0 mL) was added and the mixture was stirred at 95° C. for 20 hours under nitrogen atmosphere. The mixture was diluted water (15.0 mL) and extracted with ethyl acetate (20.0 mL×3). Combined organic layers were dried over sodium sulfate, filtered and concentrated under vacuum to give 9-(1-ethoxyvinyl)-7-methyl-4-oxo-2-(piperidin-1-yl)-4H-pyrido[1,2-a]pyrimidine-3-carbonitrile as a yellow solid (510 mg, crude). LCMS [M+1]⁺=339.0.

Step E: To a solution of 9-(1-ethoxyvinyl)-7-methyl-4-oxo-2-(piperidin-1-yl)-4H-pyrido[1,2-a]pyrimidine-3-carbonitrile (800 mg, 2.36 mmol, 1.00 eq.) in dichloromethane (4.00 mL) was added trifluoroacetic acid (6.16 g, 54.0 mmol, 4.00 mL, 22.9 eq.). The mixture was stirred at 0° C. for 2 hours. The mixture was diluted water (15.0 mL) and extracted with ethyl acetate (3×15.0 mL). Combined organic layers were dried over sodium sulfate and concentrated under vacuum to give a residue. The residue was purified by silica gel chromatography (petroleum ether/ethyl acetate 50/1, 3/1) to give 9-acetyl-7-methyl-4-oxo-2-(piperidin-1-yl)-4H-pyrido[1,2-a]pyrimidine-3-carbonitrile (480 mg, 1.50 mmol, 63.5% yield, 97.0% purity) as a yellow solid. LCMS [M+1]⁺=310.8.

Step F: To a solution of 9-acetyl-7-methyl-4-oxo-2-(piperidin-1-yl)-4H-pyrido[1,2-a]pyrimidine-3-carbonitrile (500 mg, 1.61 mmol, 1.00 eq.) in ethanol (1.00 mL) and dichloromethane (1.00 mL) was added sodium borohydride (487 mg, 12.8 mmol, 8.00 eq.). The mixture was stirred at −40° C. for 1.5 hours. The reaction mixture was quenched with ammonium chloride (5.00 mL) at 0° C. The mixture was diluted water (10.0 mL) and extracted with ethyl acetate (3×20.0 mL). Combined organic layers were dried over sodium sulfate and concentrated under vacuum to give 9-(1-hydroxyethyl)-7-methyl-4-oxo-2-(piperidin-1-yl)-4H-pyrido[1,2-a]pyrimidine-3-carbonitrile (480 mg, crude) as a yellow solid. LCMS [M+1]⁺=313.1.

Step G: To a solution of 9-(1-hydroxyethyl)-7-methyl-4-oxo-2-(piperidin-1-yl)-4H-pyrido[1,2-a]pyrimidine-3-carbonitrile (480 mg, 1.54 mmol, 1.00 eq.) in dichloromethane (4.00 mL) was added thionyl chloride (548 mg, 4.61 mmol, 3.00 eq.) at 0° C. The mixture was heat to 25° C. and stirred at 25° C. for 1 hour. The mixture was diluted water (5.00 mL) and extracted with ethyl acetate (3×5.00 mL). Combined organic layers were dried over sodium sulfate and concentrated under vacuum to give 9-(1-chloroethyl)-7-methyl-4-oxo-2-(piperidin-1-yl)-4H-pyrido[1,2-a]pyrimidine-3-carbonitrile (480 mg, crude) as a yellow solid. LCMS [M+1]⁺=331.1.

Step H: To a solution of tert-butyl 2-aminobenzoate (841 mg, 4.35 mmol, 3.00 eq.) in dimethylformamide (4.00 mL) was added 9-(1-chloroethyl)-7-methyl-4-oxo-2-(piperidin-1-yl)-4H-pyrido[1,2-a]pyrimidine-3-carbonitrile (480 mg, 1.45 mmol, 1.00 eq.), diisopropylethylamine (938 mg, 7.25 mmol, 1.26 mL, 5.00 eq.) and potassium iodide (24.1 mg, 145 μmol, 0.100 eq.). The mixture was stirred at 40° C. for 12 hours. The mixture was diluted water (30.0 mL) and extracted with ethyl acetate (3×30.0 mL). Combined organic layers were dried over sodium sulfate, filtered and concentrated under vacuum to give a residue. The residue was purified by silica gel chromatography (petroleum ether/ethyl acetate 50/1, 3/1) to give tert-butyl 2-((1-(3-cyano-7-methyl-4-oxo-2-(piperidin-1-yl)-4H-pyrido[1,2-a]pyrimidin-9-yl)ethyl)amino)benzoate (60 mg, 106 μmol, 7.29% yield, 86% purity) as a yellow solid. LCMS [M+1]⁺=488.2.

Step I: To a solution of tert-butyl 2-[1-[3-cyano-7-methyl-4-oxo-2-(1-piperidyl) pyrido[1,2-a]pyrimidin-9-yl]ethylamino]benzoate (45 mg, 92.3 μmol, 1.00 eq.) was added trifluoroacetic acid (31.6 mg, 277 μmol, 3.00 eq.) at 0° C. The mixture was heat to 25° C. and stirred at 25° C. for 1.5 hours. The mixture was concentrated and neutralized with ammonium hydroxide to pH=7. The mixture was purified by prep-HPLC (column: Waters Xbridge 150×25 mm×5 um; mobile phase: [water (NH₄HCO₃)-acetonitrile]; B %: 20%-50%, 8 min) to give 2-((1-(3-cyano-7-methyl-4-oxo-2-(piperidin-1-yl)-4H-pyrido[1,2-a]pyrimidin-9-yl)ethyl)amino)benzoic acid (5.91 mg, 13.57 μmol, 14.7% yield, 99.1% purity) as a white solid. LCMS [M+1]⁺=432.1.

¹H NMR (400 MHz, MeOD) δ=8.51 (s, 1H), 7.94-7.89 (m, 1H), 7.71 (d, J=1.6 Hz, 1H), 7.17 (t, J=6.8 Hz, 1H), 6.56 (t, J=7.2 Hz, 1H), 6.34-6.28 (m, 1H), 5.24 (q, J=7.2 Hz, 1H), 4.04-4.02 (m, 4H), 2.26 (s, 3H), 1.82-1.72 (m, 6H), 1.62 (d, J=6.4 Hz, 3H)

### Example 9-1

### 2-((1-(7-methyl-4-oxo-2-(tetrahydro-2H-pyran-4-yl)-4H-pyrido[1,2-a]pyrimidin-9-yl)ethyl)amino)benzoic acid

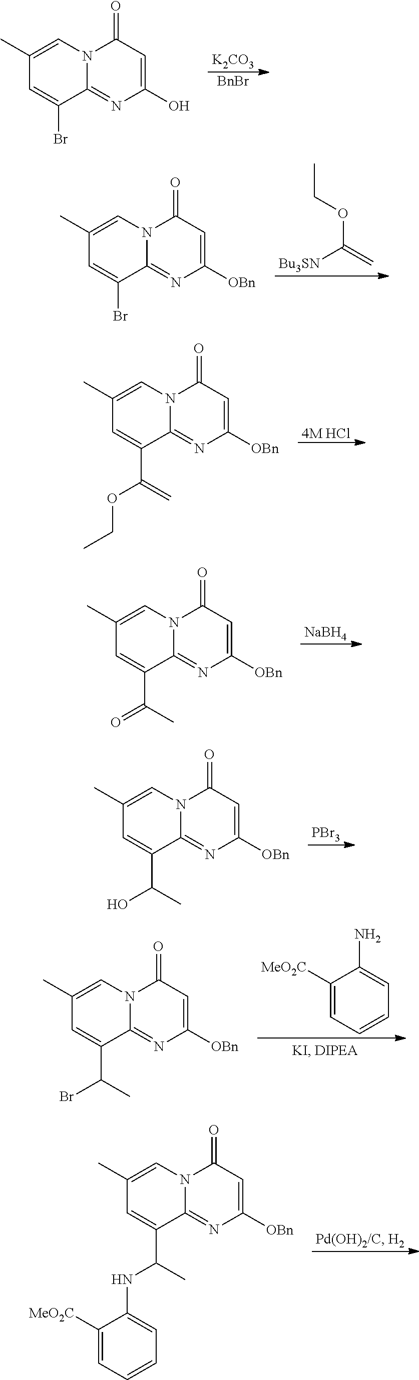

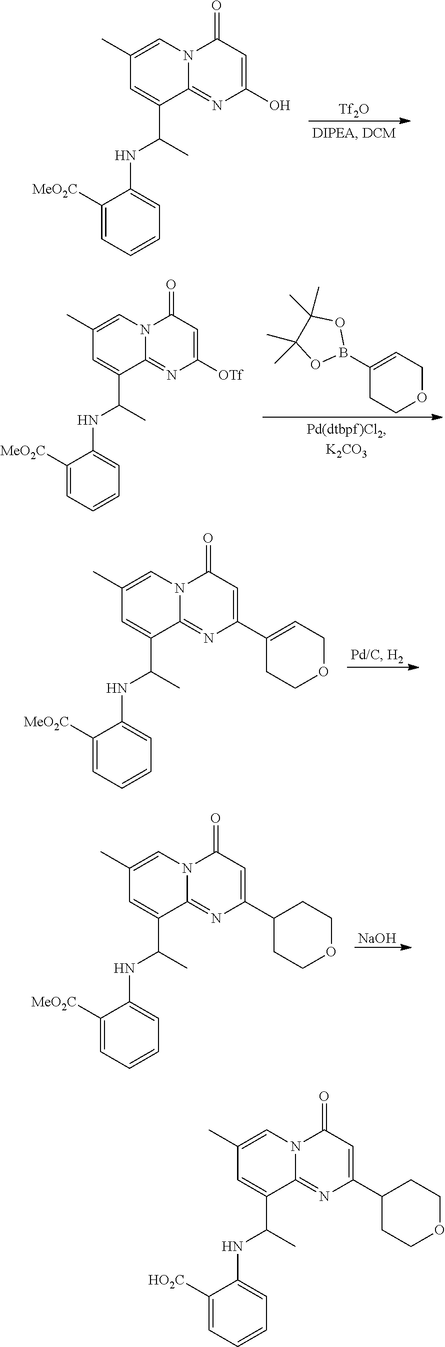

Step A: To a solution of 9-bromo-2-hydroxy-7-methyl-4H-pyrido[1,2-a]pyrimidin-4-one (80.0 g, 313 mmol, 1.00 eq.), (bromomethyl)benzene (80.4 g, 470 mmol, 55.8 mL, 1.50 eq.) in dimethylformamide (200 mL) was added potassium carbonate (130 g, 940 mmol, 3.00 eq.). The mixture was stirred at 80° C. for 2 hours. Upon completion, the reaction mixture cooled to 25° C., diluted with water (1000 mL) and extracted with ethyl acetate (500 mL×3). The combined organic layers were washed with brine (300 mL×3), dried over anhydrous sodium sulfate, filtered and concentrated under reduced pressure to give a residue. The residue was purified by flash silica gel chromatography, (eluent of 0-40% ethyl acetate in petroleum ether) to give 2-(benzyloxy)-9-bromo-7-methyl-4H-pyrido[1,2-a]pyrimidin-4-one (47.0 g, 136 mmol, 54.0% yield, 85.7% purity) as a yellow solid. LCMS [M+1]⁺=347.1.

¹H NMR (400 MHz, DMSO-d₆) δ=8.78 (s, 1H), 8.37 (d, J=16 Hz, 1H), 7.53 (br d, J=6.8 Hz, 2H), 7.38 (br d, J=7.3 Hz, 3H), 5.74 (s, 1H), 5.57-5.36 (m, 2H), 2.37 (s, 3H).

Step B: To a solution of 2-(benzyloxy)-9-bromo-7-methyl-4H-pyrido[1,2-a]pyrimidin-4-one (47.0 g, 136 mmol, 1.00 eq.), tributyl(1-ethoxyvinyl) stannane (59.0 g, 163 mmol, 55.1 mL, 1.20 eq.) was added bis(triphenylphosphine) palladium (II) dichloride (4.70 g, 6.81 mmol, 0.05 eq.) and the reaction was evacuated and backfilled three times with nitrogen. Then dioxane (250 mL) was added and the mixture was stirred at 100° C. for 12 hours under a nitrogen atmosphere. Upon completion, the reaction mixture cooled to 25° C., the reaction mixture was diluted with water (500 mL) and extracted with ethyl acetate (500 mL×3). The combined organic layers were washed with brine (200 mL×3), dried over anhydrous sodium sulfate, filtered and concentrated under reduced pressure to give 2-(benzyloxy)-9-(1-ethoxyvinyl)-7-methyl-4H-pyrido[1,2-a]pyrimidin-4-one (36.0 g, 107 mmol, 78.6% yield, 71.5% purity) as a yellow solid. LCMS [M+1]⁺=337.2.

Step C: To a solution of 2-(benzyloxy)-9-(1-ethoxyvinyl)-7-methyl-4H-pyrido[1,2-a]pyrimidin-4-one (36.0 g, 107 mmol, 1.00 eq.) in water (100 mL), tetrahydrofuran (100 mL) was added hydrochloric acid (4.00 M in water, 133 mL, 5.00 eq.). The mixture was stirred at 25° C. for two hours. Upon completion, the reaction mixture was quenched by addition sodium bicarbonate to PH=7 at 25° C., and then diluted with water (100 mL) and extracted with ethyl acetate (200 mL×3). The combined organic layers were washed with brine (200 mL×3), dried over anhydrous sodium sulfate, filtered and concentrated under reduced pressure to give a residue. The residue was purified by flash silica gel chromatography (eluent of 0˜40% ethyl acetate in petroleum ether) to give 9-acetyl-2-(benzyloxy)-7-methyl-4H-pyrido[1,2-a]pyrimidin-4-one (32.0 g, 103 mmol, 96.9% yield, 71.3% purity) as a yellow solid. LCMS [M+1]⁺=309.2.

Step D: To a solution of 9-acetyl-2-(benzyloxy)-7-methyl-4H-pyrido[1,2-a]pyrimidin-4-one (32.0 g, 103 mmol, 1.00 eq.) in dichloromethane (100 mL) and methanol (100 mL) was added sodium borohydride (7.85 g, 207 mmol, 2.00 eq.) at 0° C. The mixture was stirred at 0° C. for two hours under a nitrogen atmosphere. Upon completion, the reaction mixture was diluted with ammonium chloride (50.0 mL) and extracted with a solution (200 mL) of dichloromethane and methanol (10/1) three times. The combined organic layers were washed with brine (200 mL×3), dried over anhydrous sodium sulfate, filtered and concentrated under reduced pressure to give 2-(benzyloxy)-9-(1-hydroxyethyl)-7-methyl-4H-pyrido[1,2-a]pyrimidin-4-one (32.0 g, 103 mmol, 99.3% yield, 88.0% purity) as a yellow solid. LCMS [M+1]⁺=311.2.

Step E: To a solution 2-(benzyloxy)-9-(1-hydroxyethyl)-7-methyl-4H-pyrido[1,2-a]pyrimidin-4-one (34.0 g, 109 mmol, 1.00 eq.) in dichloromethane (200 mL) was added phosphorus tribromide (35.5 g, 131 mmol, 1.20 eq.) at 0° C., then warmed to 25° C. and stirred for two hours. Upon completion, the reaction mixture was quenched by addition of sodium bicarbonate solution to pH=7, diluted with water (50 mL) and extracted with ethyl acetate (200 mL×3). The combined organic layers were washed with brine (200 mL×3), dried over anhydrous sodium sulfate, filtered and concentrated under reduced pressure to give 2-(benzyloxy)-9-(1-bromoethyl)-7-methyl-4H-pyrido[1,2-a]pyrimidin-4-one (19.0 g, 50.9 mmol, 46.4% yield, 35.3% purity) as a yellow oil. LCMS [M+1]⁺=375.0.

Step F: To a solution of 2-(benzyloxy)-9-(1-bromoethyl)-7-methyl-4H-pyrido[1,2-a]pyrimidin-4-one (19.0 g, 50.9 mmol, 1.00 eq.), methyl 2-aminobenzoate (11.5 g, 76.3 mmol, 9.87 mL, 1.50 eq.) in dimethylformamide (100 mL) was added diisopropylethylamine (19.7 g, 152 mmol, 26.6 mL, 3.00 eq.) and potassium iodide (8.45 g, 50.9 mmol, 1.00 eq.). The mixture was stirred at 70° C. for two hours. Upon completion, the reaction mixture was cooled to 25° C., the mixture diluted with water (100 mL) and extracted with ethyl acetate (200 mL×3). The combined organic layers were washed with brine (200 mL×3), dried over anhydrous sodium sulfate, filtered and concentrated under reduced pressure to give a residue. The residue was purified by flash silica gel chromatography (0-50% Ethyl acetate in petroleum ether) to give methyl 2-((1-(2-(benzyloxy)-7-methyl-4-oxo-4H-pyrido[1,2-a]pyrimidin-9-yl)ethyl)amino)benzoate (6.00 g, 13.5 mmol, 26.6% yield, 90.2% purity) as a yellow oil. LCMS [M+1]⁺=444.3.

¹H NMR (400 MHz, DMSO-d₆) δ=8.73 (s, 1H), 8.27 (d, J=6.8 Hz, 1H), 7.82 (dt, J=1.6, 4.0 Hz, 2H), 7.57-7.44 (m, 2H), 7.42-7.34 (m, 2H), 7.34-7.28 (m, 1H), 7.26-7.17 (m, 1H), 6.63-6.49 (m, 1H), 6.35 (d, J=8.8 Hz, 1H), 5.77 (s, 1H), 5.52 (s, 2H), 5.33 (quin, J=6.4 Hz, 1H), 3.85 (s, 3H), 2.32 (d, J=0.8 Hz, 3H), 1.54 (d, J=6.8 Hz, 3H).

Step G: To a solution of methyl 2-((1-(2-(benzyloxy)-7-methyl-4-oxo-4H-pyrido[1,2-a]pyrimidin-9-yl)ethyl)amino)benzoate (5.20 g. 11.7 mmol, 1.00 eq.) in ethyl acetate (20.0 mL) was added Pd(OH) 2 on carbon (1.65 g, 11.7 mmol, 1.00 eq.). The mixture was stirred at 25° C. for 1.5 hour under hydrogen atmosphere (15 psi). Upon completion, the reaction mixture was filtered and concentrated under reduced pressure to give methyl 2-((1-(2-hydroxy-7-methyl-4-oxo-4H-pyrido[1,2-a]pyrimidin-9-yl)ethyl)amino)benzoate (3.60 g, 10.1 mmol, 86.8% yield, 83.1% purity) as a yellow oil. LCMS [M+1]⁺=354.2.

Step H: To a solution of methyl 2-((1-(2-hydroxy-7-methyl-4-oxo-4H-pyrido[1,2-a]pyrimidin-9-yl)ethyl)amino)benzoate (3.50 g, 9.90 mmol, 1.00 eq.) and trifluoromethanesulfonic anhydride (5.59 g, 19.8 mmol, 3.27 mL, 2.00 eq.) in dichloromethane (300 mL) was added diisopropylethylamine (6.40 g, 49.5 mmol, 8.63 mL, 5.00 eq.) at 0° C. Upon complet addition, the mixture was warmed to 25° C. and stirred for two hours under a nitrogen atmosphere. Upon completion, the reaction mixture was filtered, and the filtrate was concentrated under reduced pressure to give a residue. The residue was purified by flash silica gel chromatography (0-20% ethyl acetate in petroleum ether) to give methyl 2-((1-(7-methyl-4-oxo-2-(((trifluoromethyl) sulfonyl)oxy)-4H-pyrido[1,2-a]pyrimidin-9-yl)ethyl)amino)benzoate (2.00 g, 4.12 mmol, 41.6% yield, 90.3% purity) as a yellow oil. LCMS [M+1]⁺=486.2.

Step I: To a solution of methyl 2-((1-(7-methyl-4-oxo-2-(((trifluoromethyl) sulfonyl)oxy)-4H-pyrido[1,2-a]pyrimidin-9-yl)ethyl)amino)benzoate (155 mg, 319 μmol, 1.00 eq.), 2-(3,6-dihydro-2H-pyran-4-yl)-4,4,5,5-tetramethyl-1,3,2-dioxaborolane (134 mg, 638 μmol, 2.00 eq.) in dioxane (1.00 mL) and water (0.50 mL) was added potassium carbonate 957(132 mg, umol, 3.00 eq.) [1,1′-Bis(di-tert-butylphosphino) ferrocene]dichloropalladium (II) (20.8 mg, 31.9 μmol, 0.10 eq.). The mixture was stirred at 100° C. for two hours under nitrogen atmosphere. Upon completion, the reaction mixture cooled to 25° C., the solution was filtered and concentrated under reduced pressure to give a residue. The residue was purified by flash silica gel chromatography (0-30% ethyl acetate in petroleum ether) to give methyl 2-((1-(2-(3,6-dihydro-2H-pyran-4-yl)-7-methyl-4-oxo-4H-pyrido[1,2-a]pyrimidin-9-yl)ethyl)amino)benzoate (130 mg, 309 μmol, 97.0% yield, 95.8% purity) as a yellow solid. LCMS [M+1]⁺=420.2.

Step J: To a solution of methyl 2-((1-(2-(3,6-dihydro-2H-pyran-4-yl)-7-methyl-4-oxo-4H-pyrido[1,2-a]pyrimidin-9-yl)ethyl)amino)benzoate (70.0 mg, 166 μmol, l, 1.00 eq.) in ethyl acetate (5.00 mL) was added palladium on carbon (100 mg, 166 μmol, 10.0% purity, 1.00 eq.). The mixture was stirred at 25° C. for 5 minutes under a hydrogen atmosphere (15 psi). Upon completion, the reaction mixture was filtered and concentrated under reduced pressure to give methyl methyl 2-((1-(7-methyl-4-oxo-2-(tetrahydro-2H-pyran-4-yl)-4H-pyrido[1,2-a]pyrimidin-9-yl)ethyl)amino)benzoate (60.0 mg, 142 μmol, 85.3% yield, 91.9% purity) as a yellow solid. LCMS [M+1]⁺=422.2.

Step K: To a solution of methyl 2-((1-(7-methyl-4-oxo-2-(tetrahydro-2H-pyran-4-yl)-4H-pyrido[1,2-a]pyrimidin-9-yl)ethyl)amino)benzoate (60.0 mg, 142 μmol, 1.00 eq.) in methanol (1.00 mL) was added sodium hydroxide (2.00 M, 711 μL, 10.0 eq.). The mixture was stirred at 40° C. for 2 hour. Upon completion, the reaction mixture was quenched by addition hydrochloric acid (4.00 M in water) to PH=3 at 25° C., and then diluted with water (10.0 mL), filtered and the solids were dried under reduced pressure to give 2-((1-(7-methyl-4-oxo-2-(tetrahydro-2H-pyran-4-yl)-4H-pyrido[1,2-a]pyrimidin-9-yl)ethyl)amino)benzoic acid (20.0 mg, 47.1 μmol, 33.1% yield, 97.4% purity) as a white solid. LCMS [M+1]⁺=408.2.

¹H NMR (400 MHz, MeOD) δ=8.65 (s, 1H), 7.81 (dd, J=1.6, 7.8 Hz, 1H), 7.64 (d, J=1.6 Hz, 1H), 7.11-7.02 (m, 1H), 6.45 (t, J=7.4 Hz, 1H), 6.30 (s, 1H), 6.25 (d, J=8.4 Hz, 1H), 5.44 (q, J=6.8 Hz, 1H), 4.07-3.89 (m, 2H), 3.51 (dt, J=2.8, 11.6 Hz, 2H), 2.95-2.80 (s, 1H), 2.25 (d, J=0.8 Hz, 3H), 1.99-1.76 (m, 4H), 1.57 (d, J=6.8 Hz, 3H).

### Example 9-2

### 2-((1-(2-(4,4-dimethylpiperidin-1-yl)-4-oxo-4H-pyrido[1,2-a]pyrimidin-9-yl)ethyl)amino) benzoic acid

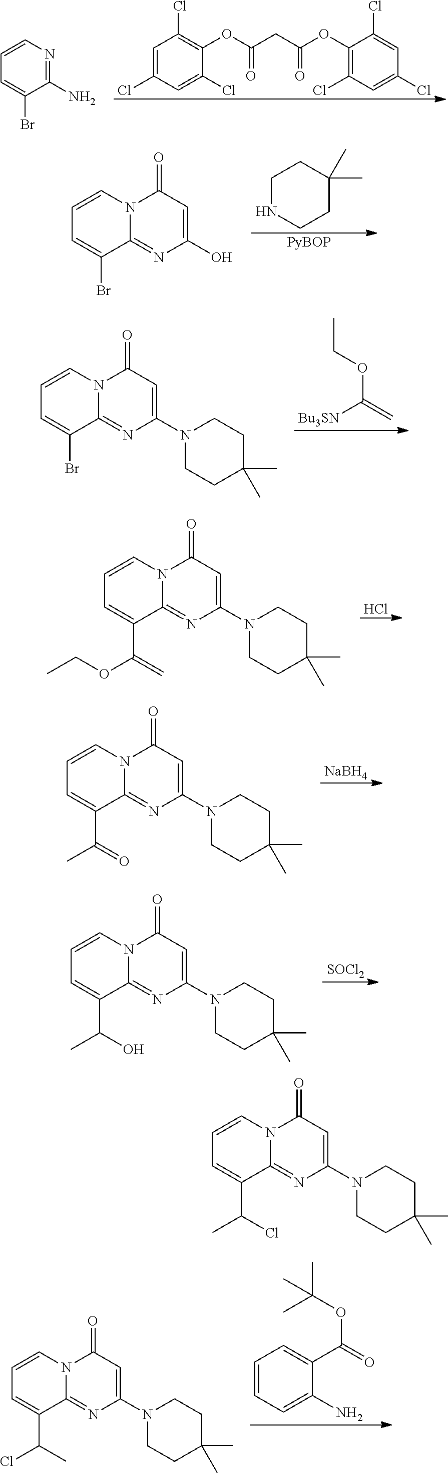

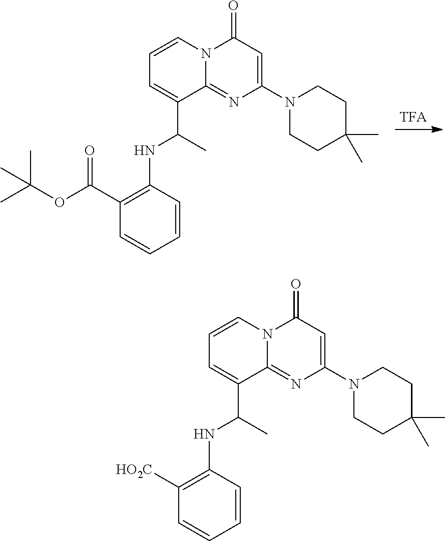 [SMILES: CC(Nc1ccccc1C(=O)OC(C)(C)C)c1cccn2c(=O)cc(N3CCC(C)(C)CC3)nc12 ; CC(Nc1ccccc1C(=O)O)c1cccn2c(=O)cc(N3CCC(C)(C)CC3)nc12]

Step A: To a solution of 3-bromopyridin-2-amine (10.0 g, 57.8 mmol, 1.00 eq.) in acetone (100 mL) was added bis(2,4,6-trichlorophenyl) malonate (28.1 g, 60.7 mmol, 1.05 eq.). The mixture was stirred at 25° C. for four hours. The reaction mixture was filtered and concentrated under reduced pressure to give a residue. The residue was triturated with ethyl acetate (20.0 mL) at 25° C. to give 9-bromo-2-hydroxy-4H-pyrido[1,2-a]pyrimidin-4-one (10.0 g, 41.5 mmol, 72% yield) was obtained as a white solid. LCMS [M+3]⁺=242.9.

¹H NMR (400 MHz, DMSO-d₆) δ=11.94-11.74 (m, 1H), 11.84 (br s, 1H), 8.89 (dd, J=1.2, 7.2 Hz, 1H), 8.34 (dd, J=1.2, 7.2 Hz, 1H), 7.12 (t, J=7.2 Hz, 1H), 5.53 (s, 1H), 5.62-5.42 (m, 1H).

Step B: To a solution of 9-bromo-2-hydroxy-4H-pyrido[1,2-a]pyrimidin-4-one (10.0 g, 41.5 mmol, 1.00 eq.) and 4,4-dimethylpiperidine (9.93 g, 66.4 mmol, 1.60 eq., HCl salt) in dimethylformamide (100 mL) was added 1,8-diazabicyclo[5.4.0]undec-7-ene (25.3 g, 166 mmol, 25.0 mL, 4.00 eq.) and benzotriazole-1-yl-oxy-tris —pyrrolidino-phosphonium hexafluorophosphate (34.5 g, 66.4 mmol, 1.60 eq.). Upon completion, the mixture was stirred at 25° C. for 12 hours. The reaction mixture was diluted with ethyl acetate (500 mL). The combined organic layers were washed with water (500 mL×2), dried over anhydrous sodium sulfate to give a residue. The residue was purified by column chromatography (SiO₂, petroleum ether/ethyl acetate=1/1 to 1/2) to give 9-bromo-2-(4,4-dimethylpiperidin-1-yl)-4H-pyrido[1,2-a]pyrimidin-4-one (10.0 g, 29.7 mmol, 71% yield) as a yellow solid. LCMS [M+3]+=338.0.

¹H NMR (400 MHz, DMSO-d₆) δ=8.72 (dd, J=1.2, 7.2 Hz, 1H), 8.17 (dd, J=1.2, 7.2 Hz, 1H), 6.89 (t, J=7.2 Hz, 1H), 5.58 (s, 1H), 3.65 (br s, 4H), 1.52-1.21 (m, 4H), 0.95 (s, 6H).

Step C: To a mixture of 9-bromo-2-(4,4-dimethylpiperidin-1-yl)-4H-pyrido[1,2-a]pyrimidin-4-one (8.00 g, 23.8 mmol, 1.00 eq.), tributyl(1-ethoxyvinyl) stannane (22.5 g, 62.2 mmol, 21.0 mL, 2.62 eq.), bis(triphenylphosphine) palladium (II) dichloride (1.67 g, 2.38 mmol, 0.10 eq.) was evacuated and backfilled with nitrogen for 3 times. Then toluene (80.0 mL) was added and the mixture was stirred at 100° C. for eight hours under a nitrogen atmosphere. After completion of the reaction, the mixture was cooled to 25° C. the mixture was quenched by addition saturated potassium fluoride aqueous solution (200 mL), then the mixture was diluted with water (500 mL) and extracted with ethyl acetate (500 mL×3). The combined organic phase was washed with brine (500 mL), dried over anhydrous sodium sulfate, filtered and concentrated under vacuum to give a residue. The residue was purified by column chromatography (SiO₂, Petroleum ether/ethyl acetate=5/1 to 2/1) to give 2-(4,4-dimethylpiperidin-1-yl)-9-(1-ethoxyvinyl)-4H-pyrido[1,2-a]pyrimidin-4-one (7.50 g. 21.1 mmol, 89% yield, 92% purity) as yellow oil. LCMS [M+1]⁺=328.2.

¹H NMR (400 MHz, DMSO-de) δ=8.76 (dd, J=1.6, 7.2 Hz, 1H), 7.87 (dd, J=1.6, 7.2 Hz, 1H), 7.01 (t, J=7.2 Hz, 1H), 5.60 (s, 1H), 5.01 (d, J=1.6 Hz, 1H), 4.61 (d, J=1.6 Hz, 1H), 3.88 (q. J=6.8 Hz, 2H), 3.62 (br s, 4H), 1.37-1.26 (m, 7H). 0.96 (s, 6H).

Step D: To a solution of 2-(4,4-dimethylpiperidin-1-yl)-9-(1-ethoxyvinyl)-4H-pyrido[1,2-a]pyrimidin-4-one (7.10 g, 21.7 mmol, 1.00 eq.) in tetrahydrofuran (75.0 mL) was added hydrochloric acid (2.00 M in water, 32.5 mL, 3.00 eq.). The mixture was stirred at 25° C. for 30 minutes. After completion of the reaction, the reaction mixture was diluted with ethyl acetate (600 mL) and washed with saturated sodium bicarbonate solution (600 ml×3). The organic layers were dried over anhydrous sodium sulfate, filtered and concentrated under reduced pressure to give a residue. The residue was purified by column chromatography (SiO₂, petroleum ether/ethyl acetate=2/1 to 1/2) to give 9-acetyl-2-(4,4-dimethylpiperidin-1-yl)-4H-pyrido[1,2-a]pyrimidin-4-one (5.80 g, 19.0 mmol, 88% yield, 98% purity) as a yellow solid. LCMS [M+1]⁺=300.1.

¹H NMR (400 MHz, DMSO-de) δ=8.86 (dd, J=1.6, 7.2 Hz, 1H), 7.94 (dd, J=1.6, 7.2 Hz, 1H), 7.06 (t, J=7.2 Hz, 1H), 5.62 (s, 1H), 3.60 (br s, 4H), 2.66 (s, 3H), 1.39-1.28 (m, 4H), 0.96 (s, 6H).

Step E: To a solution of 9-acetyl-2-(4,4-dimethylpiperidin-1-yl)-4H-pyrido[1,2-a]pyrimidin-4-one (5.50 g, 18.4 mmol, 1.00 eq.) in dichloromethane (55.0 mL) and methanol (55.0 mL) was added sodium borohydride in batches (720 mg, 19.0 mmol, 1.04 eq.) at −10° C. and stirred at −10° C. for one hour. The reaction mixture was quenched by addition ammonium chloride saturated solution 100 mL and extracted with dichloromethane/methanol (150 mL×3, 10/1). The combined organic layers were washed with brine (100 mL×2), dried over anhydrous sodium sulfate, filtered and concentrated under reduced pressure to give 2-(4,4-dimethylpiperidin-1-yl)-9-(1-hydroxyethyl)-4H-pyrido[1,2-a]pyrimidin-4-one (5.36 g, 17.1 mmol, 93% yield, 96% purity) as a yellow solid. LCMS [M+1]⁺=302.2.

¹H NMR (400 MHz, DMSO-d₆) δ=8.67 (dd, J=1.6, 7.2 Hz, 1H), 7.84 (d, J=6.4 Hz, 1H), 7.05 (t, J=6.8 Hz, 1H), 5.59 (s, 1H), 5.34 (d, J=4.4 Hz, 1H), 5.25-5.16 (m, 1H), 3.62 (br s, 4H), 1.39 (d, J=6.4 Hz, 3H), 1.37-1.31 (m, 4H), 0.96 (s, 6H).

Step F: To a solution of 2-(4,4-dimethylpiperidin-1-yl)-9-(1-hydroxyethyl)-4H-pyrido[1,2-a]pyrimidin-4-one (500 mg, 1.66 mmol, 1.00 eq.) in dichloromethane (5.00 mL) was added thionyl chloride (395 mg, 3.32 mmol, 241 μL, 2.00 eq.) at 0° C. and stirred at 0° C. for five hours. The reaction mixture was concentrated under reduced pressure to give 9-(1-chloroethyl)-2-(4,4-dimethylpiperidin-1-yl)-4H-pyrido[1,2-a]pyrimidin-4-one (500 mg, crude) as a yellow solid. LCMS [M+1]⁺=320.1.

Step G: To a solution of 9-(1-chloroethyl)-2-(4,4-dimethylpiperidin-1-yl)-4H-pyrido[1,2-a]pyrimidin-4-one (200 mg, 375 μmol, 60% purity, 1.00 eq.) in dimethylformamide (2.00 mL) was added tert-butyl 2-aminobenzoate (109 mg, 563 μmol, 103 μL, 1.50 eq.), diisopropylethylamine (97.0 mg, 750 μmol, 131 μL, 2.00 eq.), potassium iodide (125 mg, 750 μmol, 2.00 eq.) and stirred at 80° C. for 12 hours. After completion of the reaction, the mixture was cooled to 25° C. and filtered to give a filtrate, the filtrate was purified was purified by prep-HPLC (column: Phenomenex luna C18 150×25 mm×10 um; mobile phase: [water (formic acid)-acetonitrile]; B %: 90%-00%, 10 min) to provide tert-butyl 2-((1-(2-(4,4-dimethylpiperidin-1-yl)-4-oxo-4H-pyrido[1,2-a]pyrimidin-9-yl)ethyl)amino)benzoate (55.0 mg, 113 μmol, 30% yield, 98% purity) as yellow oil. LCMS [M+1]⁺=477.3.

¹H NMR (400 MHz, DMSO-d₆) δ=8.67 (dd, J=1.2, 7.2 Hz, 1H), 8.20-8.14 (m, 1H), 7.77 (dd, J=1.2, 8.0 Hz, 1H), 7.66 (d, J=6.0 Hz, 1H), 7.31-7.12 (m, 1H), 6.97 (t, J=6.8 Hz, 1H), 6.54 (t, J=7.6 Hz, 1H), 6.35 (d, J=8.4 Hz, 1H), 5.20 (br t, J=6.8 Hz, 1H), 3.68 (br s, 4H), 1.65-1.49 (m, 12H), 1.44-1.34 (m, 4H), 0.98 (s, 6H).

Step H: To a solution of tert-butyl 2-((1-(2-(4,4-dimethylpiperidin-1-yl)-4-oxo-4H-pyrido[1,2-a]pyrimidin-9-yl)ethyl)amino)benzoate (40.0 mg, 83.9 μmol, 1.00 eq.) in dichloromethane (0.50 mL) was added trifluoroacetic acid (1.54 g, 13.5 mmol, 1.00 mL, 161 eq.) at 0° C. and stirred at 25° C. for eight hours. After completion of the reaction, the reaction mixture was concentrated under reduced pressure to give a residue. Then the residue was diluted with dichloromethane (10.0 mL) and concentrated under reduced pressure to give a residue. The residue was purified by prep-HPLC (column: Phenomenex Luna C18 150×25 mm×10 um; mobile phase: [water (formic acid)-acetonitrile]; B %: 51%-81%, 9 min) to give 2-((1-(2-(4,4-dimethylpiperidin-1-yl)-4-oxo-4H-pyrido[1,2-a]pyrimidin-9-yl)ethyl)amino)benzoic acid (15.7 mg, 37.0 μmol, 44% yield, 99% purity) as a white solid. LCMS [M+1]⁺=421.2.

¹H NMR (400 MHz, DMSO-d₆) δ=12.75 (br s, 1H), 8.67 (d, J=7.2 Hz, 1H), 8.40 (br d, J=5.2 Hz, 1H), 7.80 (br d, J=8.0 Hz, 1H), 7.64 (br d, J=6.8 Hz, 1H), 7.21 (br t, J=8.0 Hz, 1H), 6.97 (t, J=7.2 Hz, 1H), 6.54 (t, J=7.6 Hz, 1H), 6.32 (br d, J=8.4 Hz, 1H), 5.65 (s, 1H), 5.38-5.02 (m, 1H), 3.68 (br s, 4H), 1.58 (br d, J=6.8 Hz, 3H), 1.43-1.30 (m, 4H), 0.98 (s, 6H).

### Example 9-3

### 2-((1-(2-(benzyloxy)-7-methyl-4-oxo-4H-pyrido[1,2-a]pyrimidin-9-yl)ethyl)amino)benzoic acid

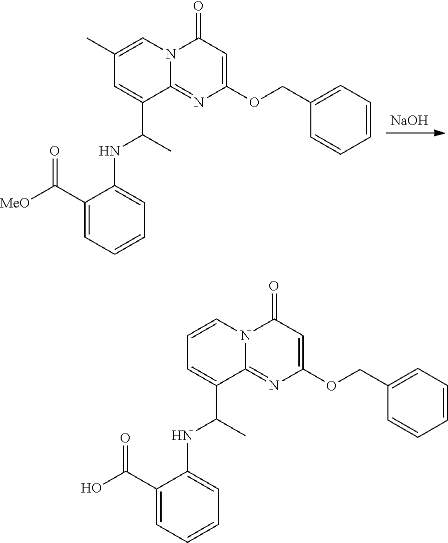

Step A: To a solution of methyl 2-((1-(2-(benzyloxy)-7-methyl-4-oxo-4H-pyrido[1,2-a]pyrimidin-9-yl)ethyl)amino)benzoate (30.0 mg, 67.6 μmol, 1.00 eq) in methanol (1.00 mL) was added sodium hydroxide (2.00 M in water, 338 μL, 10.0 eq). The mixture was stirred at 25° C. for one hour. Upon completion, The residue was purified by prep-HPLC (column: Phenomenex C_{18 75×30} mm×3 um; mobile phase: [water (formic acid)-acetonitrile];B %: 48%-78%, 7 min) to give 2-((1-(2-(benzyloxy)-7-methyl-4-oxo-4H-pyrido[1,2-a]pyrimidin-9-yl)ethyl)amino)benzoic acid (5.30 mg, 11.8 μmol, 17.5% yield, 99.2% purity) as a yellow oil. LCMS [M+1]⁺=430.2.

¹H NMR (400 MHz, DMSO-de) δ=8.73 (s, 1H), 8.66-8.48 (m, 1H), 7.82 (br d, J=7.6 Hz, 1H), 7.78 (s, 1H), 7.48 (d, J=7.2 Hz, 2H), 7.42-7.27 (m, 4H), 7.14 (br t, J=7.4 Hz, 1H), 6.53 (t, J=7.5 Hz, 1H), 6.26 (br d, J=8.3 Hz, 1H), 5.77 (s, 1H), 5.56-5.46 (m, 2H), 5.36-5.22 (m, 1H), 2.31 (s, 3H), 1.51 (br d, J=6.4 Hz, 3H).

The following compound is prepared essentially according to the procedures set forth in the above schemes and examples.

| Ex. # | Structure | Spectral Data |
|---|---|---|
| 9-4 | 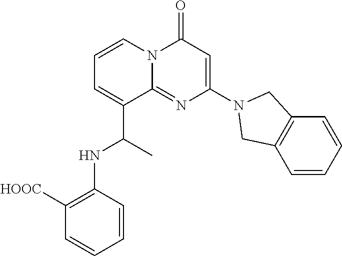 [SMILES: CC(Nc1ccccc1C(=O)O)c1cccn2c(=O)cc(N3Cc4ccccc4C3)nc12] | LCMS [M + 1]⁺ = 427.1. |
| | 2-((1-(2-(isoindolin-2-yl)-4-oxo-4H-pyrido[1,2- | |
| | a]pyrimidin-9-yl)ethyl)amino)benzoic acid | |

### Example 10-1

### (R)-2-((1-(2-(4-(4-cyanophenyl) piperazin-1-yl)-3.7-dimethyl-4-oxo-4H-pyrido[1,2-a]pyrimidin-9-yl)ethyl)amino)benzoic acid

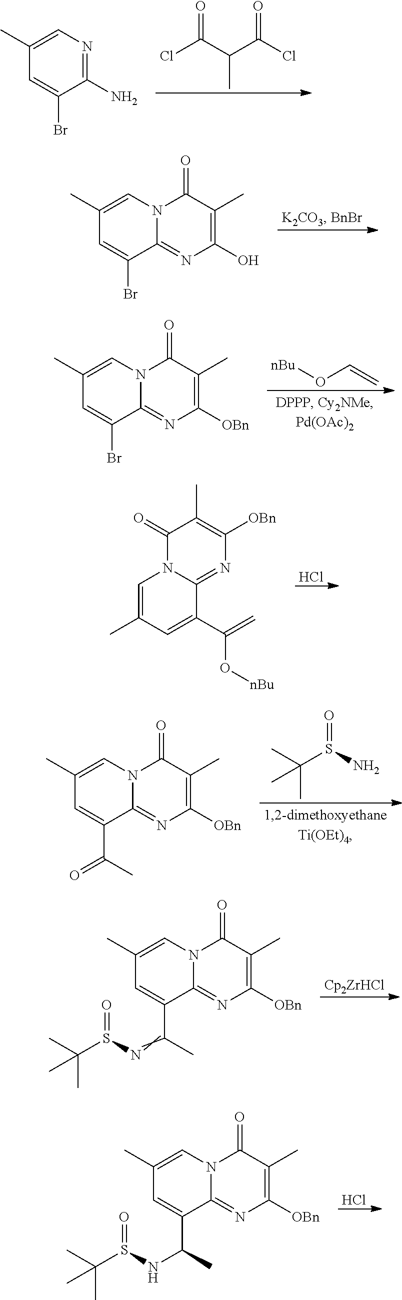

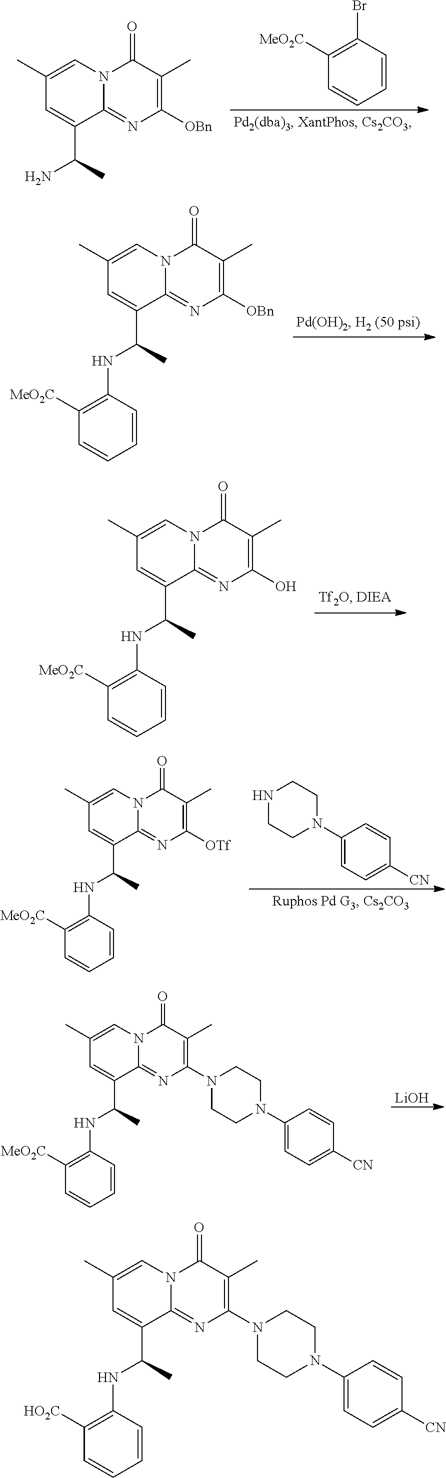

Step A: To a solution of 3-bromo-5-methylpyridin-2-amine (400 g, 2.14 mol, 1.00 eq.) in dichloromethane (3.00 L) was added 2-methylmalonyl dichloride (365 g, 2.35 mol, 1.10 eq.) drop-wise slowly at 25° C., the mixture was stirred at 25° C. for 48 hours. Upon completion the mixture was filtered, and the filter cake was washed with ethyl acetate (250 mL×2) and dried under vacuum to give 9-bromo-2-hydroxy-3,7-dimethyl-4H-pyrido[1,2-a]pyrimidin-4-one (490 g, 1.46 mol, 68.2% yield, 80.1% purity) as a white solid. LCMS [M+3]⁺=270.9.

Step B: To a solution of 9-bromo-2-hydroxy-3,7-dimethyl-4H-pyrido[1,2-a]pyrimidin-4-one (490 g, 1.82 mol, 1.00 eq.) in dimethylformamide (3.50 L) were added potassium carbonate (755 g, 5.46 mol, 3.00 eq.) and benzyl bromide (467, 2.73 mol, 324 mL, 1.50 eq.), the mixture was stirred at 100° C. for one hour. The reaction mixture was cooled to 25° C. and diluted with water (7.00 L), the suspended mixture was filtered and filter cake dried under vacuum to give 2-(benzyloxy)-9-bromo-3,7-dimethyl-4H-pyrido[1,2-a]pyrimidin-4-one (517 g, 1.36 mol, 74.6% yield, 94.4% purity) as a yellow solid. LCMS [M+3]⁺=360.9.

¹H NMR (400 MHz, CDCl₃) δ=8.72 (s, 1H), 7.79 (d, J=1.6 Hz, 1H), 7.44 (d, J=7.2 Hz, 2H), 7.31-7.20 (m, 3H), 5.49 (s, 2H), 2.29 (s, 3H), 2.08 (s, 3H).

Step C: A mixture of 2-(benzyloxy)-9-bromo-3,7-dimethyl-4H-pyrido[1,2-a]pyrimidin-4-one (410 g, 1.14 mol, 1.00 eq.), n-butyl vinyl ether (572 g, 5.71 mol, 734 mL, 5.00 eq.), Pd(OAc) 2(12.8 g, 57.1 mmol, 0.05 eq.), 1,3-bis(diphenylphosphino) propane (47.1 g, 114 mmol, 0.10 eq.) and N,N-dicyclohexylmethylamine (669 g, 3.42 mol, 726 mL, 3.00 eq.) in n-butyl alcohol (3.00 L) was evacuated and backfilled with nitrogen three times, and then the mixture was stirred at 100° C. for 12 hours under nitrogen atmosphere. Upon completion the reaction mixture was cooled to 25° C. and filtered, the filtrate was used into the next step without further purification.

Step D: To a solution of 2-(benzyloxy)-9-(1-butoxyvinyl)-3,7-dimethyl-4H-pyrido[1,2-a]pyrimidin-4-one (420 g, 1.11 mol, 1.00 eq.) in n-butyl alcohol (2.00 L) was added hydrochloric acid (12 M, 740 mL, 8.00 eq.), the mixture was stirred at 25° C. for one hour. Upon completion the mixture was filtered, and the filter cake was washed with ethyl acetate (500 mL) and dried under vacuum to give 9-acetyl-2-(benzyloxy)-3,7-dimethyl-4H-pyrido[1,2-a]pyrimidin-4-one (300 g, 83.6%) as a yellow solid. LCMS [M+1]⁺=323.3.

¹H NMR (400 MHz, DMSO-d₆) δ=8.91 (s, 1H), 8.00 (d, J=2.0 Hz, 1H), 7.49-7.30 (m, 5H), 5.48 (s, 2H), 2.71 (s, 3H), 2.41 (s, 3H), 2.05 (s, 3H).

Step E: To a solution of 9-acetyl-2-(benzyloxy)-3,7-dimethyl-4H-pyrido[1,2-a]pyrimidin-4-one (210 g, 651 mmol, 1.00 eq.) and (R)-2-methylpropane-2-sulfinamide (103 g, 847 mmol, 1.30 eq.) in tetrahydrofuran (1.60 L) were added titanium (IV) ethoxide (297 g, 1.30 mol, 270 mL, 2.00 eq.) and 1,2-dimethoxyethane (58.7 g, 651 mmol, 67.7 mL. 1.00 eq.), the mixture was stirred at 80° C. for 16 hours. After completion the reaction was quenched by water (60.0 mL) slowly and diluted with ethyl acetate (2.00 L), the resulting mixture was filtered and the filtrate was washed with brine (2.00 L), dried over anhydrous sodium sulfate, filtered and concentrated in vacuum to give (R)—N-(1-(2-(benzyloxy)-3,7-dimethyl-4-oxo-4H-pyrido[1,2-a]pyrimidin-9-yl)ethylidene)-2-methylpropane-2-sulfinamide (230 g, crude) as yellow oil which was used into the next step without further purification.

Step F: To a solution of (R)—N-(1-(2-(benzyloxy)-3,7-dimethyl-4-oxo-4H-pyrido[1,2-a]pyrimidin-9-yl)ethylidene)-2-methylpropane-2-sulfinamide (230 g, 540 mmol, 1.00 eq.) in dichloromethane (2.50 L) was added zirconocene chloride hydride (167 g, 649 mmol, 1.20 eq.) at 0° C. under nitrogen, after addition the mixture was stirred at 25° C. for one hour. The reaction was quenched with saturated aqueous ammonium chloride (2.00 L) and then exacted with dichloromethane (1.50 L×2). The combined organic phase were washed with brine (3.00 L), dried over anhydrous sodium sulfate, filtered and concentrated under vacuum to give a residue. The residue was triturated with a solution of petroleum ether/ethyl acetate (5/1, 300 mL) at 25° C. for one hour to give (R)—N—((R)-1-(2-(benzyloxy)-3,7-dimethyl-4-oxo-4H-pyrido[1,2-a]pyrimidin-9-yl)ethyl)-2-methylpropane-2-sulfinamide (160 g, 374 mmol, 69.2% yield) as a yellow solid. LCMS [M+1]⁺=428.4.

¹H NMR (400 MHz, CDCl₃) δ=8.84 (s, 1H), 7.56 (s, 1H), 7.50-7.44 (m, 2H), 7.39 (m, 2H), 7.33 (br d, J=7.2 Hz, 1H), 5.52 (s, 2H), 4.93 (m, 1H), 4.78 (br d. J=7.2 Hz, 1H), 2.41 (s, 3H), 2.20 (s, 3H), 1.59 (br d, J=6.8 Hz, 3H), 1.22 (s, 9H).

Step G: To a solution of (R)—N—((R)-1-(2-(benzyloxy)-3,7-dimethyl-4-oxo-4H-pyrido[1,2-a]pyrimidin-9-yl)ethyl)-2-methylpropane-2-sulfinamide (300 g, 702 mmol, 1.00 eq.) in dioxane (1.00 L) was added hydrochloric acid (4 M in dioxane, 526 mL, 3.00 eq.) drop-wise at 25° C., the mixture was stirred at 25° C. for one hour. Upon completion the reaction was filtered and the filter cake was triturated with ethyl acetate (2.00 L×2) for one hour at 25° C., filtered, the filter cake was dried under vacuum to give (R)-9-(1-aminoethyl)-2-(benzyloxy)-3,7-dimethyl-4H-pyrido[1,2-a]pyrimidin-4-one (85.0 g, 226 mmol, 47.9% yield, 95.8% purity, hydrochloric acid) as a yellow solid. LCMS [M+1]⁺=324.1.

¹H NMR (400 MHz, DMSO-d₆) δ=8.78 (s, 1H), 8.74 (br s, 3H), 8.10 (d, J=1.2 Hz, 1H), 7.52-7.46 (m, 2H), 7.40 (t, J=7.2 Hz, 2H), 7.35-7.29 (m, 1H), 5.63-5.39 (m, 2H), 5.04 (br s, 1H), 2.40 (s, 3H), 2.04 (s, 3H), 1.58 (d, J=6.8 Hz, 3H).

Step H: A mixture of (R)-9-(1-aminoethyl)-2-(benzyloxy)-3,7-dimethyl-4H-pyrido[1,2-a]pyrimidin-4-one (70.0 g, 195 mmol, 1.00 eq., hydrochloric acid), methyl 2-bromobenzoate (50.2 g, 233 mmol, 32.8 mL, 1.20 eq.), cesium carbonate (190 g, 584 mmol, 3.00 eq.), tris(dibenzylideneacetone) dipalladium (0) (17.8 g, 19.5 mmol, 0.10 eq.) and 4,5-bis(diphenylphosphino)-9,9-dimethylxanthene (22.5 g, 38.9 mmol, 0.20 eq.) was evacuated and backfilled with nitrogen 3 times, then dioxane (1.00 L) was added and the mixture was stirred at 100° C. for 4 hours under nitrogen atmosphere. Upon completion the reaction mixture was cooled to 25° C. and filtered, the filtrate was concentrated under reduced pressure to give a residue. The residue was purified by column chromatography (SiO₂, petroleum ether/ethyl acetate=10/1 to 2/1) to give methyl (R)-2-((1-(2-(benzyloxy)-3,7-dimethyl-4-oxo-4H-pyrido[1,2-a]pyrimidin-9-yl)ethyl)amino)benzoate (77.0 g, 166 mmol, 85.1% yield, 98.4% purity, e.e 99.9%) as yellow oil. LCMS [M+1]⁺=458.2.

¹H NMR (400 MHz, CDCl₃) δ=8.70 (s, 1H), 8.18 (br d, J=5.6 Hz, 1H), 7.85 (dd, J=1.6, 8.0 Hz, 1H), 7.48 (d, J=1.6 Hz, 1H), 7.38 (d, J=7.2 Hz, 2H), 7.27 (t, J=7.6 Hz, 2H), 7.22-7.18 (m, 1H), 7.04 (m, 1H), 6.59-6.41 (m, 1H), 6.08 (d, J=8.4 Hz, 1H), 5.49 (d, J=1.6 Hz, 2H), 5.37-5.21 (m, 1H), 3.84 (s, 3H), 2.22 (s, 3H), 2.17 (s, 3H), 1.48 (d, J=6.8 Hz, 3H).

Step I: To a solution of (R)-2-((1-(2-(benzyloxy)-3,7-dimethyl-4-oxo-4H-pyrido[1,2-a]pyrimidin-9-yl)ethyl)amino)benzoate (34.0 g, 74.3 mmol, 1.00 eq.) in ethyl acetate (350 mL) was added palladium (II) hydroxide on carbon (15.7 g, 22.3 mmol, 20% purity, 0.30 eq.) under nitrogen atmosphere, the suspension was purged with hydrogen 3 times, then the mixture was stirred at 25° C. for 2 hours under hydrogen (50 psi). After completion the reaction mixture was filtered and the filtrate was concentrated under reduced pressure to give methyl (R)-2-((1-(2-hydroxy-3,7-dimethyl-4-oxo-4H-pyrido[1,2-a]pyrimidin-9-yl)ethyl)amino)benzoate (25.0 g, 68.1 mmol, 91.6% yield) as a yellow solid. LCMS [M+1]+=368.1.

Step J: To a solution of methyl (R)-2-((1-(2-hydroxy-3,7-dimethyl-4-oxo-4H-pyrido[1,2-a]pyrimidin-9-yl)ethyl)amino)benzoate (25.0 g, 68.1 mmol, 1.00 eq.) and diisopropylethylamine (17.6 g, 136 mmol, 23.7 mL, 2.00 eq.) in dichloromethane (250 mL) was added trifluoromethanesulfonic anhydride (23.0 g, 81.7 mmol, 13.5 mL, 1.20 eq.) drop wise at 0° C. After addition, the mixture was stirred at 0° C. for 30 minutes. The reaction was concentrated under vacuum to give a residue. This residue was purified by column chromatography (SiO₂, petroleum ether/ethyl acetate=10/1 to 5/1) to give methyl (R)-2-((1-(3,7-dimethyl-4-oxo-2-(((trifluoromethyl) sulfonyl)oxy)-4H-pyrido[1,2-a]pyrimidin-9-yl)ethyl)amino)benzoate (12.0 g, 23.2 mmol, 34.1% yield, 92.2% purity) as a yellow gum. LCMS [M+1]⁺=500.1.

¹H NMR (400 MHz, CDCl₃) δ=8.77 (s, 1H), 8.28 (br d, J=5.6 Hz, 1H), 7.95 (dd, J=1.6, 8.0 Hz, 1H), 7.75 (d, J=1.6 Hz, 1H), 7.23-7.14 (m, 1H), 6.61 (m, 1H), 6.25 (d, J=8.4 Hz, 1H), 5.36 (t, J=6.4 Hz, 1H), 3.94 (s, 3H), 2.38 (s, 3H), 2.30 (s, 3H), 1.65 (d, J=6.4 Hz, 3H).

Step K: A mixture of methyl (R)-2-((1-(3,7-dimethyl-4-oxo-2-(((trifluoromethyl) sulfonyl)oxy)-4H-pyrido[1,2-a]pyrimidin-9-yl)ethyl)amino)benzoate (70.0 mg, 140 μmol, 1.00 eq.), 4-(piperazin-1-yl)benzonitrile (39.4 mg, 210 μmol, 1.50 eq.), (2-Dicyclohexylphosphino-2′,6′-diisopropoxy-1,1′-biphenyl) [2-(2′-amino-1,1′-biphenyl)]palladium (II) methanesulfonate (11.7 mg, 14.0 μmol, 0.10 eq.) and cesium carbonate (137 mg, 420 μmol, 3.00 eq.) was evacuated and backfilled with nitrogen 3 times, then in dioxane (1.00 mL) was added and the mixture was stirred at 100° C. for 3 hours under nitrogen atmosphere. After cooling, the reaction mixture was diluted with water (3.00 mL) at 25° C., then extracted with ethyl acetate 15.0 mL (5.00 mL×3). The combined organic layers were washed with brine 15.0 mL (5.00 mL×3), dried over anhydrous sodium sulfate, filtered and concentrated under reduced pressure to give a residue. The residue was purified by preparative TLC (petroleum ether/ethyl acetate=1/2) to give methyl (R)-2-((1-(2-(4-(4-cyanophenyl) piperazin-1-yl)-3,7-dimethyl-4-oxo-4H-pyrido[1,2-a]pyrimidin-9-yl)ethyl)amino)benzoate (41.0 mg, 60.7 μmol, 43.3% yield, 79.5% purity) as a yellow oil. LCMS [M+1]⁺=537.4.

Step L: To a solution of methyl (R)-2-((1-(2-(4-(4-cyanophenyl) piperazin-1-yl)-3,7-dimethyl-4-oxo-4H-pyrido[1,2-a]pyrimidin-9-yl)ethyl)amino)benzoate (30.0 mg, 55.9 μmol, 1.00 eq.) in tetrahydrofuran (0.25 mL) and methanol (0.50 mL) and water (0.10 mL) was added lithium hydroxide (11.7 mg, 280 μmol, 5.00 eq.). The mixture was stirred at 40° C. for 2 hours. After cooling, the reaction mixture was directly concentrated under vacuum to remove the solvent, then the pH was adjusted to 6 with 1M HCl aqueous solution. The crude product was slurried in water and collected by filtration, then the filter cake was purified by prep-HPLC (formic acid condition, column: Phenomenex luna C18 150×25 mm×10 um; mobile phase: [water (formic acid)-acetonitrile]; gradient: 52%-82% B over 10 min) to give (R)-2-((1-(2-(4-(4-cyanophenyl) piperazin-1-yl)-3,7-dimethyl-4-oxo-4H-pyrido[1,2-a]pyrimidin-9-yl)ethyl)amino)benzoic acid (16.0 mg, 30.6 μmol, 54.7% yield, 99.8% purity) as a white solid. LCMS [M+1]⁺=523.4.

¹H NMR (400 MHz, DMSO-d₆) δ=8.60 (d, J=1.6 Hz, 1H), 8.44 (br d, J=5.2 Hz, 1H), 7.80 (dd, J=1.6, 8.0 Hz, 1H), 7.65-7.60 (m, 2H), 7.59 (s, 1H), 7.26-7.16 (m, 1H), 7.06 (d, J=8.8 Hz, 2H), 6.61-6.49 (m, 1H), 6.41 (d, J=8.8 Hz, 1H), 5.32 (q, J=6.4 Hz, 1H), 3.64-3.58 (m, 4H), 3.56-3.51 (m, 4H), 2.28 (d, J=0.8 Hz, 3H), 2.13 (s, 3H), 1.61 (d, J=6.8 Hz, 3H).

### Examples 10-2 to 10-148

The following compounds are prepared essentially according to the procedures set forth in the above schemes and examples.

| Ex. # | Structure | Spectral Data |
|---|---|---|
| 10-2 | 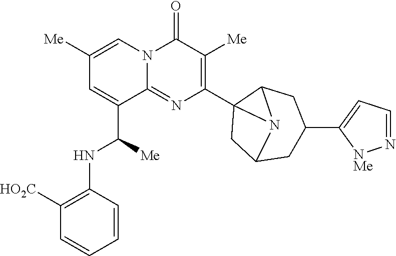 [SMILES: Cc1cc([C@@H](C)Nc2ccccc2C(=O)O)c2nc(C34CC5CC(c6ccnn6C)CC3N54)c(C)c(=O)n2c1] 2-(((1R)-1-(3,7-dimethyl-2-(3-(1-methyl-1H- pyrazol-5-yl)-8-azabicyclo[3.2.1]octan-8-yl)-4-oxo- 4H-pyrido[1,2-a]pyrimidin-9- yl)ethyl)amino)benzoic acid | |
| 10-3 | 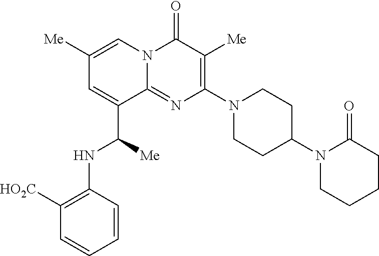 [SMILES: Cc1cc([C@@H](C)Nc2ccccc2C(=O)O)c2nc(N3CCC(N4CCCCC4=O)CC3)c(C)c(=O)n2c1] (R)-2-((1-(3,7-dimethyl-4-oxo-2-(2-oxo-[1,4′- bipiperidin]-1′-yl)-4H-pyrido[1,2-a]pyrimidin-9- yl)ethyl)amino)benzoic acid | |
| 10-4 | 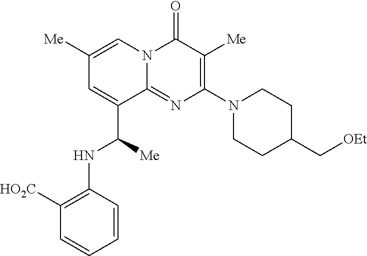 [SMILES: CCOCC1CCN(c2nc3c([C@@H](C)Nc4ccccc4C(=O)O)cc(C)cn3c(=O)c2C)CC1] (R)-2-((1-(2-(4-(ethoxymethyl)piperidin-1-yl)-3,7- dimethyl-4-oxo-4H-pyrido[1,2-a]pyrimidin-9- yl)ethyl)amino)benzoic acid | |
| 10-5 | 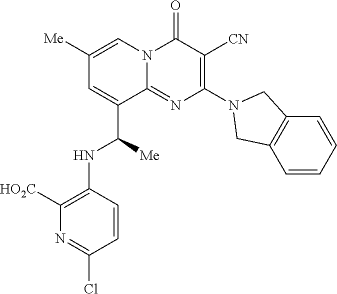 [SMILES: Cc1cc([C@@H](C)Nc2ccc(Cl)nc2C(=O)O)c2nc(N3Cc4ccccc4C3)c(C#N)c(=O)n2c1] (R)-3-((1-(3-cyano-2-(isoindolin-2-yl)-7-methyl-4- oxo-4H-pyrido[1,2-a]pyrimidin-9-yl)ethyl)amino)-6- methylpicolinic acid | LCMS [M + 1]⁺ = 481.3 |
| 10-6 | 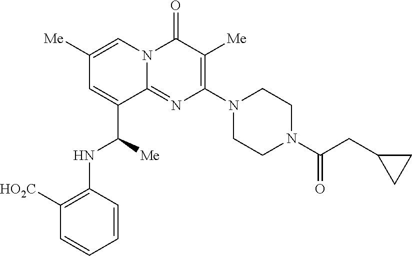 [SMILES: Cc1cc([C@@H](C)Nc2ccccc2C(=O)O)c2nc(N3CCN(C(=O)CC4CC4)CC3)c(C)c(=O)n2c1] (R)-2-((1-(2-(4-(2-cyclopropylacetyl)piperazin-1- yl)-3,7-dimethyl-4-oxo-4H-pyrido[1,2-a]pyrimidin- 9-yl)ethyl)amino)benzoic acid | |
| 10-7 | 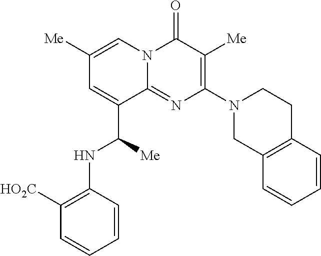 [SMILES: Cc1cc([C@@H](C)Nc2ccccc2C(=O)O)c2nc(N3CCc4ccccc4C3)c(C)c(=O)n2c1] (R)-2-((1-(2-(3,4-dihydroisoquinolin-2(1H)-yl)-3,7- dimethyl-4-oxo-4H-pyrido[1,2-a]pyrimidin-9- yl)ethyl)amino)benzoic acid | |
| 10-8 | 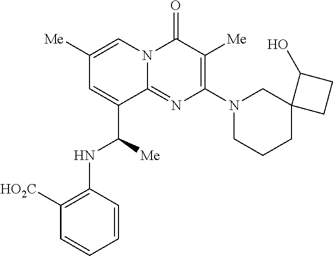 [SMILES: Cc1cc([C@@H](C)Nc2ccccc2C(=O)O)c2nc(N3CCCC4(CCC4O)C3)c(C)c(=O)n2c1] 2-(((1R)-1-(2-(1-hydroxy-6-azaspiro[3.5]nonan-6- yl)-3,7-dimethyl-4-oxo-4H-pyrido[1,2-a]pyrimidin- 9-yl)ethyl)amino)benzoic acid | |
| 10-9 | 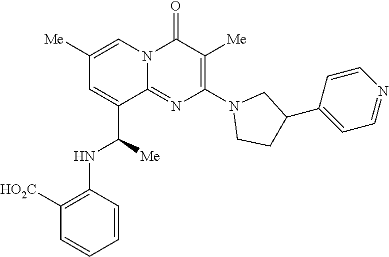 [SMILES: Cc1cc([C@@H](C)Nc2ccccc2C(=O)O)c2nc(N3CCC(c4ccncc4)C3)c(C)c(=O)n2c1] 2-(((1R)-1-(3,7-dimethyl-4-oxo-2-(3-(pyridin-4- yl)pyrrolidin-1-yl)-4H-pyrido[1,2-a]pyrimidin-9- yl)ethyl)amino)benzoic acid | |
| 10-10 | 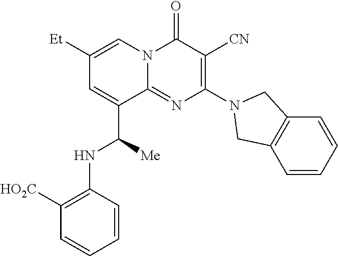 [SMILES: CCc1cc([C@@H](C)Nc2ccccc2C(=O)O)c2nc(N3Cc4ccccc4C3)c(C#N)c(=O)n2c1] (R)-2-((1-(3-cyano-7-ethyl-2-(isoindolin-2-yl)-4- oxo-4H-pyrido[1,2-a]pyrimidin-9- yl)ethyl)amino)benzoic acid | LCMS [M + 1]⁺ = 480.3 |
| 10-11 | 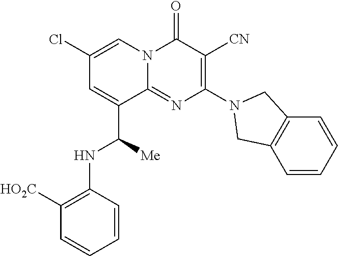 [SMILES: C[C@@H](Nc1ccccc1C(=O)O)c1cc(Cl)cn2c(=O)c(C#N)c(N3Cc4ccccc4C3)nc12] (R)-2-((1-(7-chloro-3-cyano-2-(isoindolin-2-yl)-4- oxo-4H-pyrido[1,2-a]pyrimidin-9- yl)ethyl)amino)benzoic acid | LCMS [M + 1]⁺ = 486.2. |
| 10-12 | 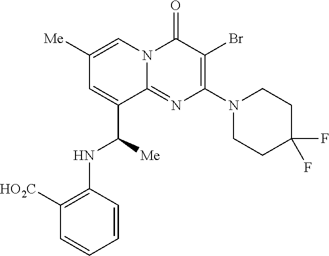 [SMILES: Cc1cc([C@@H](C)Nc2ccccc2C(=O)O)c2nc(N3CCC(F)(F)CC3)c(Br)c(=O)n2c1] (R)-2-((1-(3-bromo-2-(4,4-difluoropiperidin-1-yl)-7- methyl-4-oxo-4H-pyrido[1,2-a]pyrimidin-9- yl)ethyl)amino)benzoic acid | LCMS [M + 1]⁺ = 522.8. |
| 10-13 | 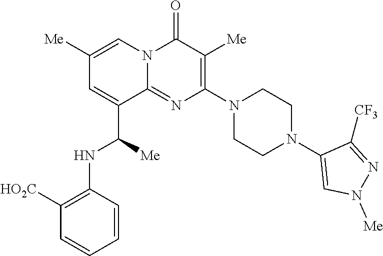 [SMILES: Cc1cc([C@@H](C)Nc2ccccc2C(=O)O)c2nc(N3CCN(c4cn(C)nc4C(F)(F)F)CC3)c(C)c(=O)n2c1] (R)-2-((1-(3,7-dimethyl-2-(4-(1-methyl-3- (trifluoromethyl)-1H-pyrazol-4-yl)piperazin-1-yl)-4- oxo-4H-pyrido[1,2-a]pyrimidin-9- yl)ethyl)amino)benzoic acid | |
| 10-14 | 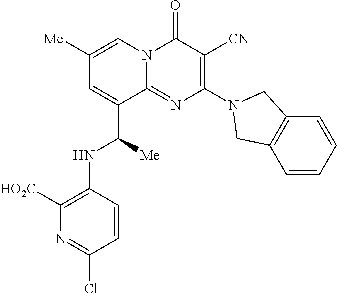 [SMILES: Cc1cc([C@@H](C)Nc2ccc(Cl)nc2C(=O)O)c2nc(N3Cc4ccccc4C3)c(C#N)c(=O)n2c1] (R)-6-chloro-3-((1-(3-cyano-2-(isoindolin-2-yl)-7- methyl-4-oxo-4H-pyrido[1,2-a]pyrimidin-9- yl)ethyl)amino)picolinic acid | |
| 10-15 | 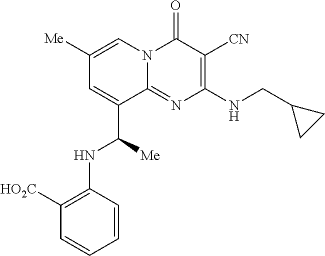 [SMILES: Cc1cc([C@@H](C)Nc2ccccc2C(=O)O)c2nc(NCC3CC3)c(C#N)c(=O)n2c1] (R)-2-((1-(3-cyano-2-((cyclopropylmethyl)amino)- 7-methyl-4-oxo-4H-pyrido[1,2-a]pyrimidin-9- yl)ethyl)amino)benzoic acid | |
| 10-16 | 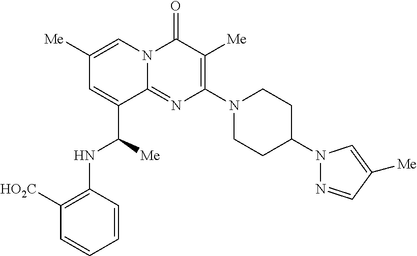 [SMILES: Cc1cnn(C2CCN(c3nc4c([C@@H](C)Nc5ccccc5C(=O)O)cc(C)cn4c(=O)c3C)CC2)c1] (R)-2-((1-(3,7-dimethyl-2-(4-(4-methyl-1H-pyrazol- 1-yl)piperidin-1-yl)-4-oxo-4H-pyrido[1,2- a]pyrimidin-9-yl)ethyl)amino)benzoic acid | |
| 10-17 | 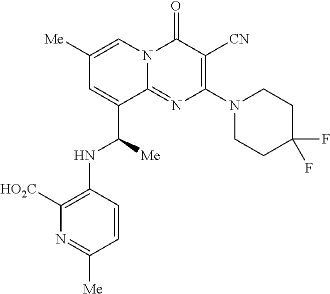 [SMILES: Cc1cc([C@@H](C)Nc2ccc(C)nc2C(=O)O)c2nc(N3CCC(F)(F)CC3)c(C#N)c(=O)n2c1] (R)-3-((1-(3-cyano-2-(4,4-difluoropiperidin-1-yl)-7- methyl-4-oxo-4H-pyrido[1,2-a]pyrimidin-9- yl)ethyl)amino)-6-methylpicolinic acid | |
| 10-18 | 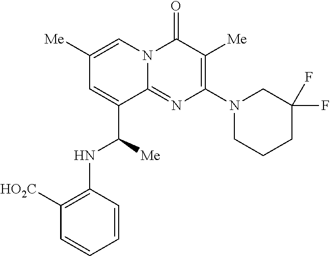 [SMILES: Cc1cc([C@@H](C)Nc2ccccc2C(=O)O)c2nc(N3CCCC(F)(F)C3)c(C)c(=O)n2c1] (R)-2-((1-(2-(3,3-difluoropiperidin-1-yl)-3,7- dimethyl-4-oxo-4H-pyrido[1,2-a]pyrimidin-9- yl)ethyl)amino)benzoic acid | |
| 10-19 | 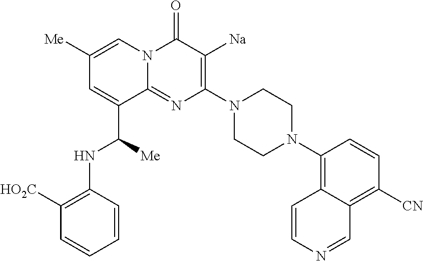 (R)-2-((1-(2-(4-(8-cyanoisoquinolin-5-yl)piperazin- 1-yl)-3,7-dimethyl-4-oxo-4H-pyrido[1,2- a]pyrimidin-9-yl)ethyl)amino)benzoic acid | |
| 10-20 | 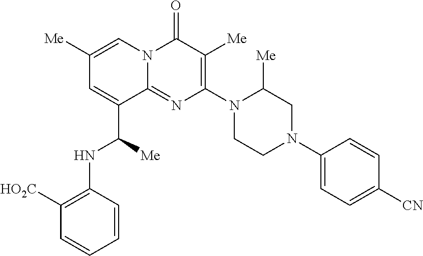 [SMILES: Cc1cc([C@@H](C)Nc2ccccc2C(=O)O)c2nc(N3CCN(c4ccc(C#N)cc4)CC3C)c(C)c(=O)n2c1] 2-(((1R)-1-(2-(4-(4-cyanophenyl)-2- methylpiperazin-1-yl)-3,7-dimethyl-4-oxo-4H- pyrido[1,2-a]pyrimidin-9-yl)ethyl)amino)benzoic acid | |
| 10-21 | 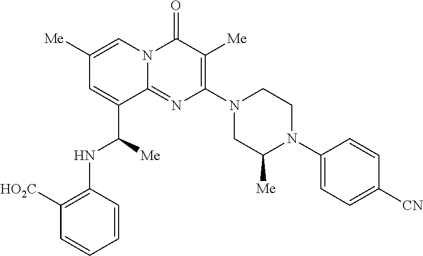 [SMILES: Cc1cc([C@@H](C)Nc2ccccc2C(=O)O)c2nc(N3CCN(c4ccc(C#N)cc4)[C@@H](C)C3)c(C)c(=O)n2c1] 2-(((R)-1-(2-((S)-4-(4-cyanophenyl)-3- methylpiperazin-1-yl)-3,7-dimethyl-4-oxo-4H- pyrido[1,2-a]pyrimidin-9-yl)ethyl)amino)benzoic acid | |
| 10-22 | 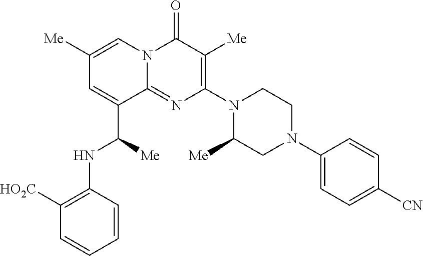 [SMILES: Cc1cc([C@@H](C)Nc2ccccc2C(=O)O)c2nc(N3CCN(c4ccc(C#N)cc4)C[C@H]3C)c(C)c(=O)n2c1] 2-(((R)-1-(2-((R)-4-(4-cyanophenyl)-2- methylpiperazin-1-yl)-3,7-dimethyl-4-oxo-4H- pyrido[1,2-a]pyrimidin-9-yl)ethyl)amino)benzoic acid | |
| 10-23 | 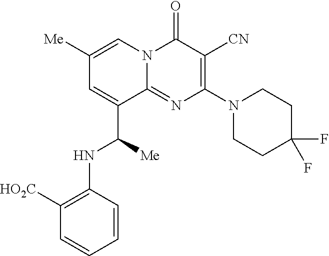 [SMILES: Cc1cc([C@@H](C)Nc2ccccc2C(=O)O)c2nc(N3CCC(F)(F)CC3)c(C#N)c(=O)n2c1] (R)-2-((1-(3-cyano-2-(4,4-difluoropiperidin-1-yl)- 7-methyl-4-oxo-4H-pyrido[1,2-a]pyrimidin-9- yl)ethyl)amino)benzoic acid | ¹H NMR (400 MHz, CD₃OD) δ = 8.57 (s, 1H), 7.92 (dd, J = 1.2, 8.0 Hz, 1H), 7.78 (d, J = 2.0 Hz, 1H), 7.24-7.14 (m, 1H), 6.57 (t, J = 7.6 Hz, 1H), 6.35 (d, J = 8.4 Hz, 1H), 5.27 (q, J = 6.8 Hz, 1H), 4.19-4.09 (m, 4H), 2.29 (s, 3H), 2.25-2.06 (m, 4H), 1.64 (d, J = 6.4 Hz, 3H). LCMS [M + 1]⁺ = 468.2. |
| 10-24 | 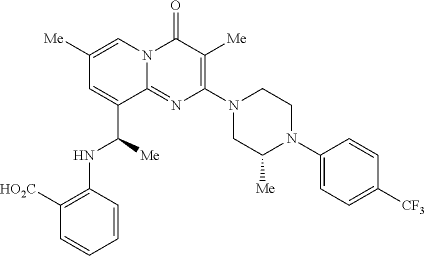 [SMILES: Cc1cc([C@@H](C)Nc2ccccc2C(=O)O)c2nc(N3CCN(c4ccc(C(F)(F)F)cc4)[C@H](C)C3)c(C)c(=O)n2c1] 2-(((R)-1-(2-((R)-4-(4-cyanophenyl)-3- methylpiperazin-1-yl)-3,7-dimethyl-4-oxo-4H- pyrido[1,2-a]pyrimidin-9-yl)ethyl)amino)benzoic acid | LCMS [M + 1]⁺ = 537.3 |
| 10-25 | 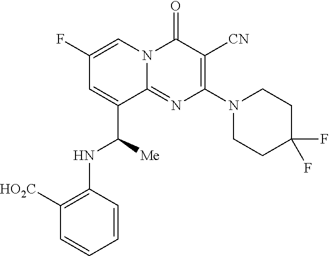 [SMILES: C[C@@H](Nc1ccccc1C(=O)O)c1cc(F)cn2c(=O)c(C#N)c(N3CCC(F)(F)CC3)nc12] (R)-2-((1-(3-cyano-2-(4,4-difluoropiperidin-1-yl)- 7-fluoro-4-oxo-4H-pyrido[1,2-a]pyrimidin-9- yl)ethyl)amino)benzoic acid | LCMS [M + 1]⁺ = 472.3 |
| 10-26 | 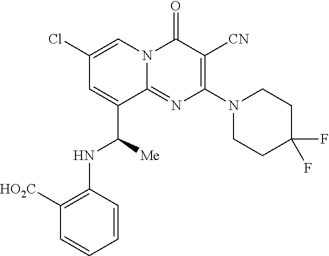 [SMILES: C[C@@H](Nc1ccccc1C(=O)O)c1cc(Cl)cn2c(=O)c(C#N)c(N3CCC(F)(F)CC3)nc12] (R)-2-((1-(7-chloro-3-cyano-2-(4,4- difluoropiperidin-1-yl)-4-oxo-4H-pyrido[1,2- a]pyrimidin-9-yl)ethyl)amino)benzoic acid | LCMS [M + 1]⁺ = 488.2 |
| 10-27 | 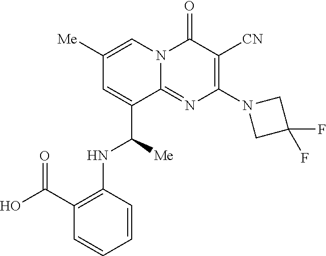 [SMILES: Cc1cc([C@@H](C)Nc2ccccc2C(=O)O)c2nc(N3CC(F)(F)C3)c(C#N)c(=O)n2c1] (R)-2-((1-(3-cyano-2-(3,3-difluoroazetidin-1-yl)-7- methyl-4-oxo-4H-pyrido[1,2-a]pyrimidin-9- yl)ethyl)amino)benzoic acid | HRMS: Calculated for C₂₂H₁₉F₂N₅O₃ [M + 1]⁺ = 440.1529; found 440.1539. |
| 10-28 | 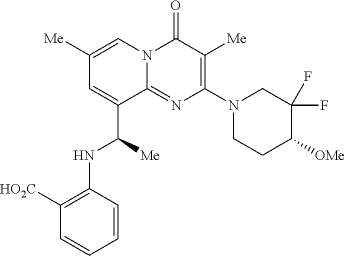 [SMILES: CO[C@@H]1CCN(c2nc3c([C@@H](C)Nc4ccccc4C(=O)O)cc(C)cn3c(=O)c2C)CC1(F)F] 2-(((R)-1-(2-((R)-3,3-difluoro-4-methoxypiperidin- 1-yl)-3,7-dimethyl-4-oxo-4H-pyrido[1,2- a]pyrimidin-9-yl)ethyl)amino)benzoic acid | LCMS [M + 1]⁺ = 487.3 |
| 10-29 | 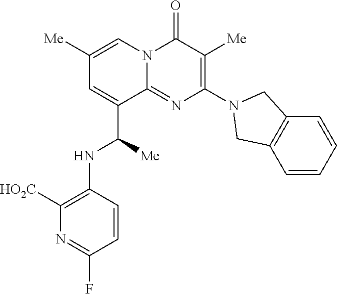 [SMILES: Cc1cc([C@@H](C)Nc2ccc(F)nc2C(=O)O)c2nc(N3Cc4ccccc4C3)c(C)c(=O)n2c1] (R)-6-fluoro-3-((1-(2-(isoindolin-2-yl)-3,7- dimethyl-4-oxo-4H-pyrido[1,2-a]pyrimidin-9- yl)ethyl)amino)picolinic acid | LCMS [M + 1]⁺ = 474.2 |
| 10-30 | 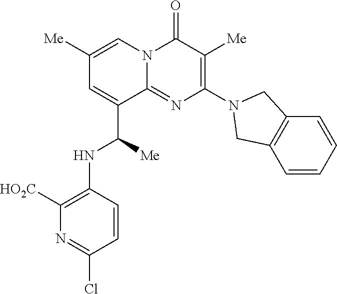 [SMILES: Cc1cc([C@@H](C)Nc2ccc(Cl)nc2C(=O)O)c2nc(N3Cc4ccccc4C3)c(C)c(=O)n2c1] (R)-6-chloro-3-((1-(2-(isoindolin-2-yl)-3,7- dimethyl-4-oxo-4H-pyrido[1,2-a]pyrimidin-9- yl)ethyl)amino)picolinic acid | LCMS [M + 1]⁺ = 490.3 |
| 10-31 | 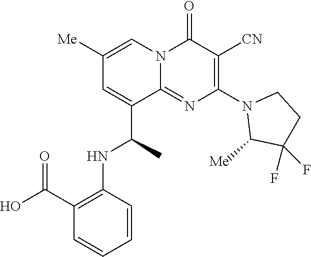 [SMILES: Cc1cc([C@@H](C)Nc2ccccc2C(=O)O)c2nc(N3CCC(F)(F)[C@@H]3C)c(C#N)c(=O)n2c1] 2-(((R)-1-(3-cyano-2-((S)-3,3-difluoro-2- methylpyrrolidin-1-yl)-7-methyl-4-oxo-4H- pyrido[1,2-a]pyrimidin-9-yl)ethyl)amino)benzoic acid | LCMS [M + 1]⁺ = 468.2 |
| 10-32 | 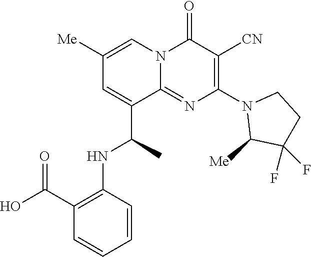 [SMILES: Cc1cc([C@@H](C)Nc2ccccc2C(=O)O)c2nc(N3CCC(F)(F)[C@H]3C)c(C#N)c(=O)n2c1] 2-(((R)-1-(3-cyano-2-((R)-3,3-difluoro-2- methylpyrrolidin-1-yl)-7-methyl-4-oxo-4H- pyrido[1,2-a]pyrimidin-9-yl)ethyl)amino)benzoic acid | LCMS [M + 1]⁺ = 468.2 |
| 10-33 | 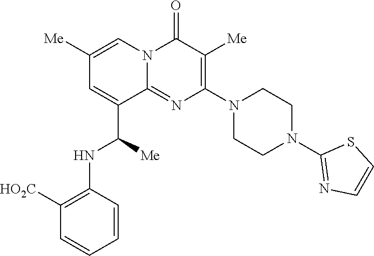 [SMILES: Cc1cc([C@@H](C)Nc2ccccc2C(=O)O)c2nc(N3CCN(c4nccs4)CC3)c(C)c(=O)n2c1] (R)-2-((1-(3,7-dimethyl-4-oxo-2-(4-(thiazol-2- yl)piperazin-1-yl)-4H-pyrido[1,2-a]pyrimidin-9- yl)ethyl)amino)benzoic acid | LCMS [M + 1]⁺ = 505.2 |
| 10-34 | 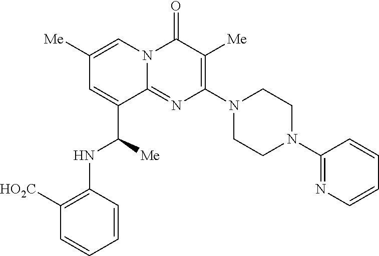 [SMILES: Cc1cc([C@@H](C)Nc2ccccc2C(=O)O)c2nc(N3CCN(c4ccccn4)CC3)c(C)c(=O)n2c1] (R)-2-((1-(3,7-dimethyl-4-oxo-2-(4-(pyridin-2- yl)piperazin-1-yl)-4H-pyrido[1,2-a]pyrimidin-9- yl)ethyl)amino)benzoic acid | LCMS [M + 1]⁺ = 499.2 |
| 10-35 | 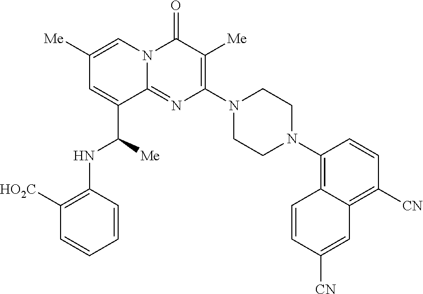 [SMILES: Cc1cc([C@@H](C)Nc2ccccc2C(=O)O)c2nc(N3CCN(c4ccc(C#N)c5cc(C#N)ccc45)CC3)c(C)c(=O)n2c1] (R)-2-((1-(2-(4-(4,6-dicyanonaphthalen-1- yl)piperazin-1-yl)-3,7-dimethyl-4-oxo-4H- pyrido[1,2-a]pyrimidin-9-yl)ethyl)amino)benzoic acid | LCMS [M + 1]⁺ = 598.3 |
| 10-36 | 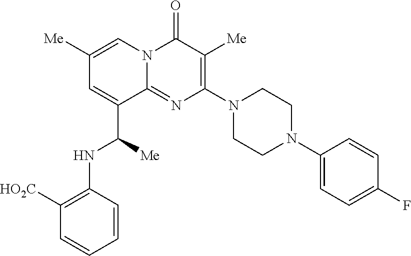 [SMILES: Cc1cc([C@@H](C)Nc2ccccc2C(=O)O)c2nc(N3CCN(c4ccc(F)cc4)CC3)c(C)c(=O)n2c1] (R)-2-((1-(2-(4-(4-fluorophenyl)piperazin-1-yl)- 3,7-dimethyl-4-oxo-4H-pyrido[1,2-a]pyrimidin-9- yl)ethyl)amino)benzoic acid | LCMS [M + 1]⁺ = 516.3 |
| 10-37 | 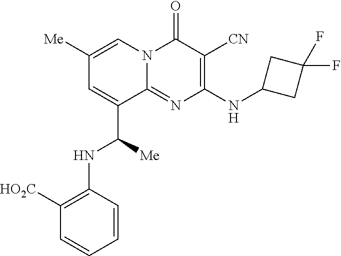 [SMILES: Cc1cc([C@@H](C)Nc2ccccc2C(=O)O)c2nc(NC3CC(F)(F)C3)c(C#N)c(=O)n2c1] (R)-2-((1-(3-cyano-2-((3,3- difluorocyclobutyl)amino)-7-methyl-4-oxo-4H- pyrido[1,2-a]pyrimidin-9-yl)ethyl)amino)benzoic acid | LCMS [M + 1]⁺ = 454.2 |
| 10-38 | 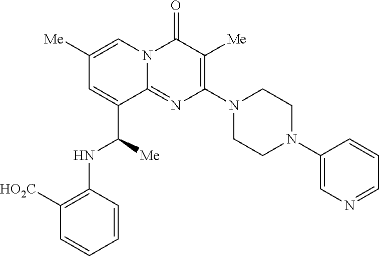 [SMILES: Cc1cc([C@@H](C)Nc2ccccc2C(=O)O)c2nc(N3CCN(c4cccnc4)CC3)c(C)c(=O)n2c1] (R)-2-((1-(3,7-dimethyl-4-oxo-2-(4-(pyridin-3- yl)piperazin-1-yl)-4H-pyrido[1,2-a]pyrimidin-9- yl)ethyl)amino)benzoic acid | ¹H NMR (400 MHz, CDCl₃) δ: 8.75 (s, 1H), 8.67 (br d, J = 1.1 Hz, 1H), 8.07 (br d, J = 4.4 Hz, 1H), 7.97 (d, J = 8.3 Hz, 1H), 7.62-7.75 (m, 2H), 7.57 (s, 1H), 7.20-7.25 (m, 1H), 6.59 (t, J = 7.6 Hz, 1H), 6.41 (d, J = 8.3 Hz, 1H), 5.14 (br d, J = 6.1 Hz, 1H), 3.76 (br d, J = 13.3 Hz, 2H), 3.64-3.71 (m, 2H), 3.53-3.64 (m, 5H), 2.36 (s, 4H), 2.26 (s, 3H), 1.73 (d, J = 6.4 Hz, 3H). LCMS [M + 1]⁺ = 499.30. |
| 10-39 | 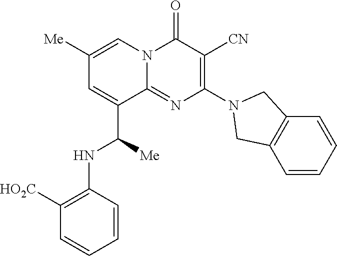 [SMILES: Cc1cc([C@@H](C)Nc2ccccc2C(=O)O)c2nc(N3Cc4ccccc4C3)c(C#N)c(=O)n2c1] (R)-2-((1-(3-cyano-7-fluoro-2-(isoindolin-2-yl)-4- oxo-4H-pyrido[1,2-a]pyrimidin-9- yl)ethyl)amino)benzoic acid | LCMS [M + 1]⁺ = 470.2 |
| 10-40 | 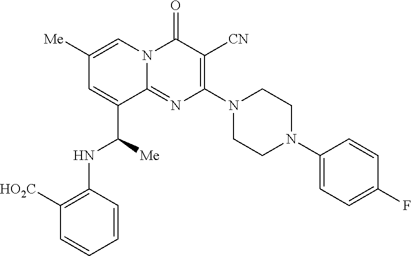 [SMILES: Cc1cc([C@@H](C)Nc2ccccc2C(=O)O)c2nc(N3CCN(c4ccc(F)cc4)CC3)c(C#N)c(=O)n2c1] (R)-2-((1-(3-cyano-2-(4-(4- fluorophenyl)piperazin-1-yl)-7-methyl-4-oxo-4H- pyrido[1,2-a]pyrimidin-9-yl)ethyl)amino)benzoic acid | LCMS [M + 1]⁺ = 527.3 |
| 10-41 | 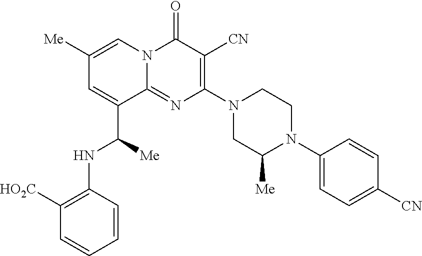 [SMILES: Cc1cc([C@@H](C)Nc2ccccc2C(=O)O)c2nc(N3CCN(c4ccc(C#N)cc4)[C@@H](C)C3)c(C#N)c(=O)n2c1] 2-(((R)-1-(3-cyano-2-((S)-4-(4-cyanophenyl)-3- methylpiperazin-1-yl)-7-methyl-4-oxo-4H- pyrido[1,2-a]pyrimidin-9-yl)ethyl)amino)benzoic acid | LCMS [M + 1]⁺ = 548.4 |
| 10-42 | 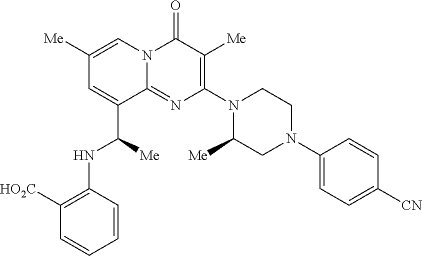 [SMILES: Cc1cc([C@@H](C)Nc2ccccc2C(=O)O)c2nc(N3CCN(c4ccc(C#N)cc4)C[C@H]3C)c(C)c(=O)n2c1] 2-(((R)-1-(2-((R)-4-(4-cyanophenyl)-2- methylpiperazin-1-yl)-3,7-dimethyl-4-oxo-4H- pyrido[1,2-a]pyrimidin-9-yl)ethyl)amino)benzoic acid | LCMS [M + 1]⁺ = 537.3 |
| 10-43 | 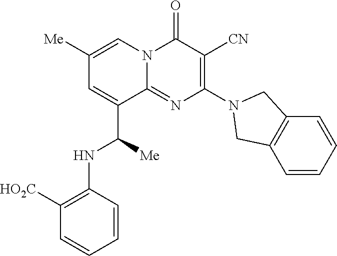 [SMILES: Cc1cc([C@@H](C)Nc2ccccc2C(=O)O)c2nc(N3Cc4ccccc4C3)c(C#N)c(=O)n2c1] (R)-2-((1-(3-cyano-2-(isoindolin-2-yl)-7-methyl-4- oxo-4H-pyrido[1,2-a]pyrimidin-9- yl)ethyl)amino)benzoic acid | LCMS [M + 1]⁺ = 466.3 |
| 10-44 | 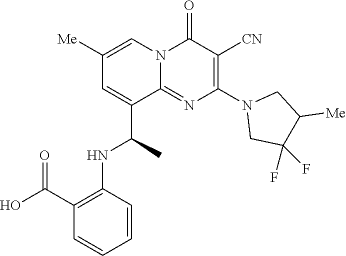 [SMILES: Cc1cc([C@@H](C)Nc2ccccc2C(=O)O)c2nc(N3CC(C)C(F)(F)C3)c(C#N)c(=O)n2c1] 2-(((1R)-1-(3-cyano-2-(3,3-difluoro-4- methylpyrrolidin-1-yl)-7-methyl-4-oxo-4H- pyrido[1,2-a]pyrimidin-9-yl)ethyl)amino)benzoic acid | LCMS [M + 1]⁺ = 468.2 |
| 10-45 | 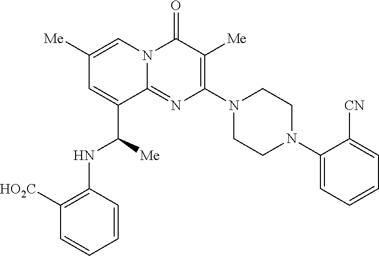 [SMILES: Cc1cc([C@@H](C)Nc2ccccc2C(=O)O)c2nc(N3CCN(c4ccccc4C#N)CC3)c(C)c(=O)n2c1] (R)-2-((1-(2-(4-(2-cyanophenyl)piperazin-1-yl)- 3,7-dimethyl-4-oxo-4H-pyrido[1,2-a]pyrimidin-9- yl)ethyl)amino)benzoic acid | LCMS [M + 1]⁺ = 523.5 |
| 10-46 | 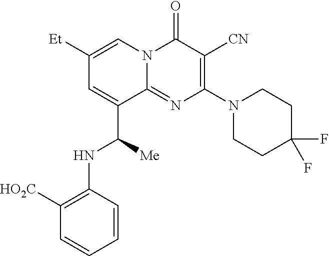 [SMILES: CCc1cc([C@@H](C)Nc2ccccc2C(=O)O)c2nc(N3CCC(F)(F)CC3)c(C#N)c(=O)n2c1] (R)-2-((1-(3-cyano-2-(4,4-difluoropiperidin-1-yl)- 7-ethyl-4-oxo-4H-pyrido[1,2-a]pyrimidin-9- yl)ethyl)amino)benzoic acid | LCMS [M + 1]⁺ = 482.2 |
| 10-47 | 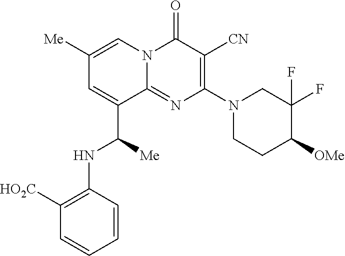 [SMILES: CO[C@H]1CCN(c2nc3c([C@@H](C)Nc4ccccc4C(=O)O)cc(C)cn3c(=O)c2C#N)CC1(F)F] 2-(((R)-1-(3-cyano-2-((S)-3,3-difluoro-4- methoxypiperidin-1-yl)-7-methyl-4-oxo-4H- pyrido[1,2-a]pyrimidin-9-yl)ethyl)amino)benzoic acid OR 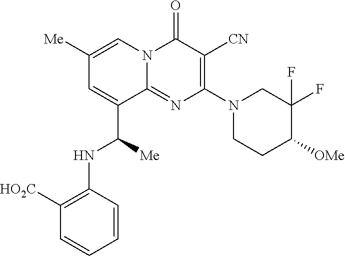 [SMILES: CO[C@@H]1CCN(c2nc3c([C@@H](C)Nc4ccccc4C(=O)O)cc(C)cn3c(=O)c2C#N)CC1(F)F] 2-(((R)-1-(3-cyano-2-((R)-3,3-difluoro-4- methoxypiperidin-1-yl)-7-methyl-4-oxo-4H- pyrido[1,2-a]pyrimidin-9-yl)ethyl)amino)benzoic acid | LCMS [M + 1]⁺ = 498.2 |
| 10-48 | 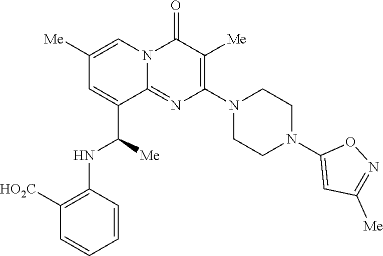 [SMILES: Cc1cc([C@@H](C)Nc2ccccc2C(=O)O)c2nc(N3CCN(c4cc(C)no4)CC3)c(C)c(=O)n2c1] (R)-2-((1-(3,7-dimethyl-2-(4-(3-methylisoxazol-5- yl)piperazin-1-yl)-4-oxo-4H-pyrido[1,2- a]pyrimidin-9-yl)ethyl)amino)benzoic acid | LCMS [M + 1]⁺ = 503.2 |
| 10-49 | 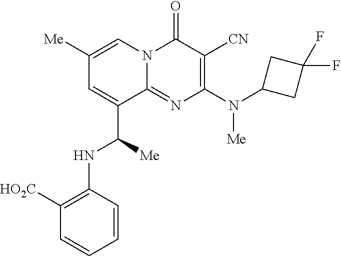 [SMILES: Cc1cc([C@@H](C)Nc2ccccc2C(=O)O)c2nc(N(C)C3CC(F)(F)C3)c(C#N)c(=O)n2c1] (R)-2-((1-(3-cyano-2-((3,3- difluorocyclobutyl)(methyl)amino)-7-methyl-4- oxo-4H-pyrido[1,2-a]pyrimidin-9- yl)ethyl)amino)benzoic acid | LCMS [M + 1]⁺ = 468.2 |
| 10-50 | 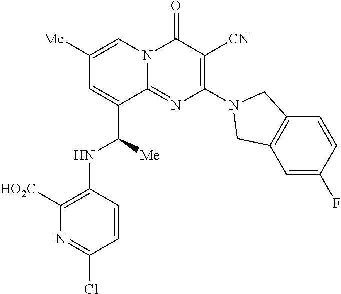 [SMILES: Cc1cc([C@@H](C)Nc2ccc(Cl)nc2C(=O)O)c2nc(N3Cc4ccc(F)cc4C3)c(C#N)c(=O)n2c1] (R)-6-chloro-3-((1-(3-cyano-2-(5-fluoroisoindolin- 2-yl)-7-methyl-4-oxo-4H-pyrido[1,2-a]pyrimidin-9- yl)ethyl)amino)picolinic acid | LCMS [M + 1]⁺ = 519.2 |
| 10-51 | 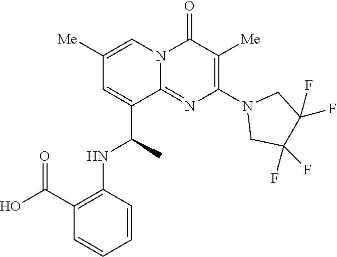 [SMILES: Cc1cc([C@@H](C)Nc2ccccc2C(=O)O)c2nc(N3CC(F)(F)C(F)(F)C3)c(C)c(=O)n2c1] (R)-2-((1-(3,7-dimethyl-4-oxo-2-(3,3,4,4- tetrafluoropyrrolidin-1-yl)-4H-pyrido[1,2- a]pyrimidin-9-yl)ethyl)amino)benzoic acid | LCMS [M + 1]⁺ = 479.2 |
| 10-52 | 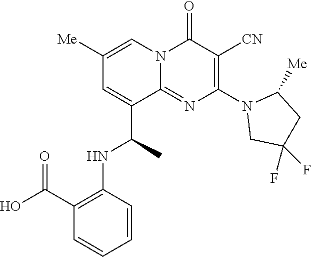 [SMILES: Cc1cc([C@@H](C)Nc2ccccc2C(=O)O)c2nc(N3CC(F)(F)C[C@H]3C)c(C#N)c(=O)n2c1] 2-(((R)-1-(3-cyano-2-((R)-4,4-difluoro-2- methylpyrrolidin-1-yl)-7-methyl-4-oxo-4H- pyrido[1,2-a]pyrimidin-9-yl)ethyl)amino)benzoic acid OR 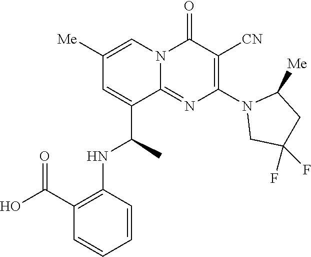 [SMILES: Cc1cc([C@@H](C)Nc2ccccc2C(=O)O)c2nc(N3CC(F)(F)C[C@@H]3C)c(C#N)c(=O)n2c1] 2-(((R)-1-(3-cyano-2-((S)-4,4-difluoro-2- methylpyrrolidin-1-yl)-7-methyl-4-oxo-4H- pyrido[1,2-a]pyrimidin-9-yl)ethyl)amino)benzoic acid | LCMS [M + 1]⁺ = 468.2 |
| 10-53 and 10-54 | 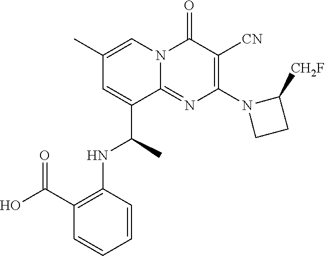 2-(((R)-1-(3-cyano-2-((R)-2- (fluoromethyl)azetidin-1-yl)-7-methyl-4-oxo-4H- pyrido[1,2-a]pyrimidin-9-yl)ethyl)amino)benzoic acid AND 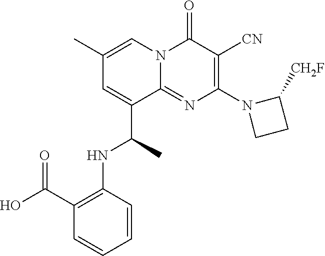 2-(((R)-1-(3-cyano-2-((S)-2- (fluoromethyl)azetidin-1-yl)-7-methyl-4-oxo-4H- pyrido[1,2-a]pyrimidin-9-yl)ethyl)amino)benzoic acid | SFC prior to saponification to give: Acid from first eluting isomer: LCMS [M + 1]⁺ = 436.2. Acid from second eluting isomer: LCMS [M + 1]⁺ = 436.2. |
| 10-55 | 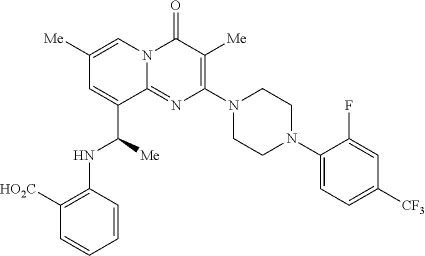 [SMILES: Cc1cc([C@@H](C)Nc2ccccc2C(=O)O)c2nc(N3CCN(c4ccc(C(F)(F)F)cc4F)CC3)c(C)c(=O)n2c1] (R)-2-((1-(2-(4-(4-cyano-2- fluorophenyl)piperazin-1-yl)-3,7-dimethyl-4-oxo- 4H-pyrido[1,2-a]pyrimidin-9- yl)ethyl)amino)benzoic acid | LCMS [M + 1]⁺ = 541.2 |
| 10-56 | 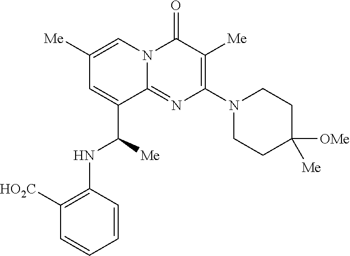 [SMILES: COC1(C)CCN(c2nc3c([C@@H](C)Nc4ccccc4C(=O)O)cc(C)cn3c(=O)c2C)CC1] (R)-2-((1-(2-(4-methoxy-4-methylpiperidin-1-yl)- 3,7-dimethyl-4-oxo-4H-pyrido[1,2-a]pyrimidin-9- yl)ethyl)amino)benzoic acid | LCMS [M + 1]⁺ = 465.1 |
| 10-57 | 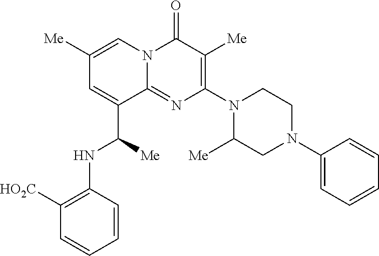 [SMILES: Cc1cc([C@@H](C)Nc2ccccc2C(=O)O)c2nc(N3CCN(c4ccccc4)CC3C)c(C)c(=O)n2c1] 2-(((1R)-1-(3-cyano-7-methyl-2-(2-methyl-4- phenylpiperazin-1-yl)-4-oxo-4H-pyrido[1,2- a]pyrimidin-9-yl) ethyl)amino)benzoic acid | LCMS [M + 1]⁺ = 523.3 |
| 10-58 | 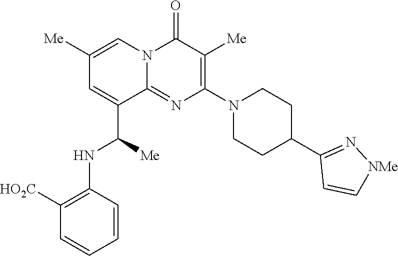 [SMILES: Cc1cc([C@@H](C)Nc2ccccc2C(=O)O)c2nc(N3CCC(c4ccn(C)n4)CC3)c(C)c(=O)n2c1] (R)-2-((1-(3,7-dimethyl-2-(4-(1-methyl-1H- pyrazol-3-yl)piperidin-1-yl)-4-oxo-4H-pyrido[1,2- a]pyrimidin-9-yl)ethyl)amino)benzoic acid | LCMS [M + 1]⁺ = 501.3 |
| 10-59 | 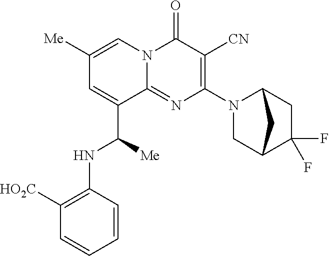 [SMILES: Cc1cc([C@@H](C)Nc2ccccc2C(=O)O)c2nc(N3C[C@@H]4C[C@H]3CC4(F)F)c(C#N)c(=O)n2c1] 2-(((R)-1-(3-cyano-2-((1S,4S)-5,5-difluoro-2- azabicyclo[2.2.1]heptan-2-yl)-7-methyl-4-oxo- 4H-pyrido[1,2-a]pyrimidin-9- yl)ethyl)amino)benzoic acid OR 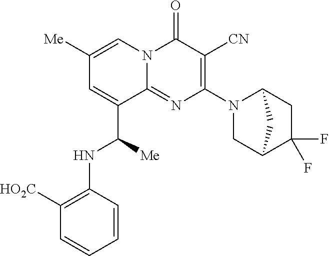 [SMILES: Cc1cc([C@@H](C)Nc2ccccc2C(=O)O)c2nc(N3C[C@H]4C[C@@H]3CC4(F)F)c(C#N)c(=O)n2c1] 2-(((R)-1-(3-cyano-2-((1R,4R)-5,5-difluoro-2- azabicyclo[2.2.1]heptan-2-yl)-7-methyl-4-oxo- 4H-pyrido[1,2-a]pyrimidin-9- yl)ethyl)amino)benzoic acid | LCMS [M + 1]⁺ = 480.3 |
| 10-60 | 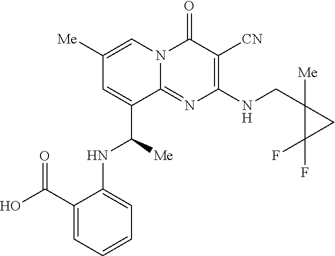 [SMILES: Cc1cc([C@@H](C)Nc2ccccc2C(=O)O)c2nc(NCC3(C)CC3(F)F)c(C#N)c(=O)n2c1] 2-(((1R)-1-(3-cyano-2-(((2,2-difluoro-1- methylcyclopropyl)methyl)amino)-7-methyl-4- oxo-4H-pyrido[1,2-a]pyrimidin-9- yl)ethyl)amino)benzoic acid | LCMS [M + 1]⁺ = 468.3 |
| 10-61 | 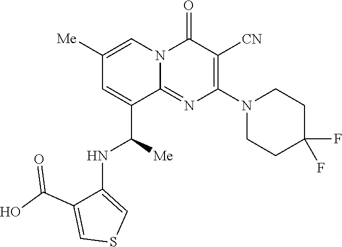 [SMILES: Cc1cc([C@@H](C)Nc2cscc2C(=O)O)c2nc(N3CCC(F)(F)CC3)c(C#N)c(=O)n2c1] (R)-4-((1-(3-cyano-2-(4,4-difluoropiperidin-1-yl)- 7-methyl-4-oxo-4H-pyrido[1,2-a]pyrimidin-9- yl)ethyl)amino)thiophene-3-carboxylic acid | LCMS [M + 1]⁺ = 474.2 |
| 10-62 | 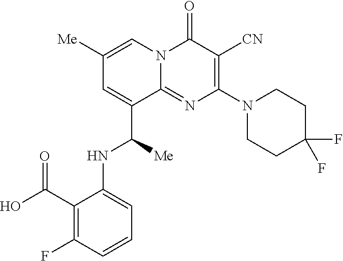 [SMILES: Cc1cc([C@@H](C)Nc2cccc(F)c2C(=O)O)c2nc(N3CCC(F)(F)CC3)c(C#N)c(=O)n2c1] (R)-2-((1-(3-cyano-2-(4,4-difluoropiperidin-1-yl)- 7-methyl-4-oxo-4H-pyrido[1,2-a]pyrimidin-9- yl)ethyl)amino)-6-fluorobenzoic acid | LCMS [M + 1]⁺ = 486.2 |
| 10-63 | 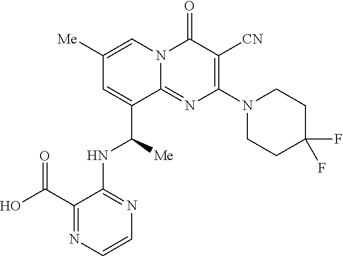 [SMILES: Cc1cc([C@@H](C)Nc2nccnc2C(=O)O)c2nc(N3CCC(F)(F)CC3)c(C#N)c(=O)n2c1] (R)-3-((1-(3-cyano-2-(4,4-difluoropiperidin-1-yl)- 7-methyl-4-oxo-4H-pyrido[1,2-a]pyrimidin-9- yl)ethyl)amino)pyrazine-2-carboxylic acid | LCMS [M + 1]⁺ = 470.0 |
| 10-64 | 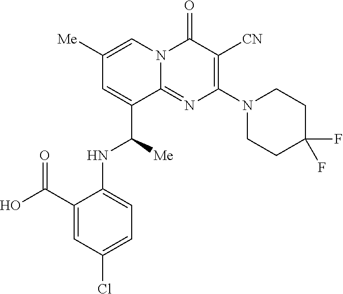 [SMILES: Cc1cc([C@@H](C)Nc2ccc(Cl)cc2C(=O)O)c2nc(N3CCC(F)(F)CC3)c(C#N)c(=O)n2c1] (R)-5-chloro-2-((1-(3-cyano-2-(4,4- difluoropiperidin-1-yl)-7-methyl-4-oxo-4H- pyrido[1,2-a]pyrimidin-9-yl)ethyl)amino)benzoic acid | LCMS [M + 1]⁺ = 502.1 |
| 10-65 | 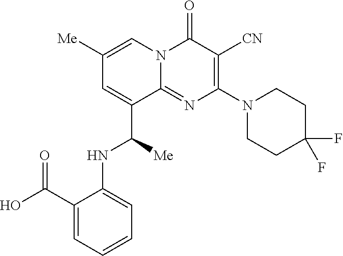 [SMILES: Cc1cc([C@@H](C)Nc2ccccc2C(=O)O)c2nc(N3CCC(F)(F)CC3)c(C#N)c(=O)n2c1] (R)-2-((1-(3-cyano-2-(4,4-difluorocyclohexyl)-7- methyl-4-oxo-4H-pyrido[1,2-a]pyrimidin-9- yl)ethyl)amino)benzoic acid | LCMS [M + 1]⁺ = 467.2 |
| 10-66 | 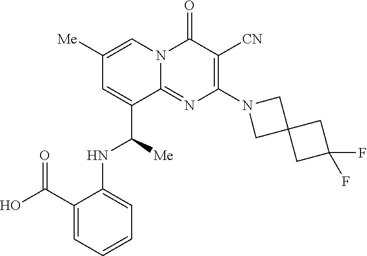 [SMILES: Cc1cc([C@@H](C)Nc2ccccc2C(=O)O)c2nc(N3CC4(C3)CC(F)(F)C4)c(C#N)c(=O)n2c1] (R)-2-((1-(3-cyano-2-(6,6-difluoro-2- azaspiro[3.3]heptan-2-yl)-7-methyl-4-oxo-4H- pyrido[1,2-a]pyrimidin-9-yl)ethyl)amino)benzoic acid | LCMS [M + 1]⁺ = 480.3 |
| 10-67 | 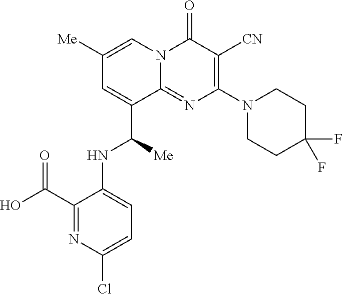 [SMILES: Cc1cc([C@@H](C)Nc2ccc(Cl)nc2C(=O)O)c2nc(N3CCC(F)(F)CC3)c(C#N)c(=O)n2c1] (R)-6-chloro-3-((1-(3-cyano-2-(4,4- difluoropiperidin-1-yl)-7-methyl-4-oxo-4H- pyrido[1,2-a]pyrimidin-9-yl)ethyl)amino)picolinic acid | LCMS [M + 1]⁺ = 503.3 |
| 10-68 | 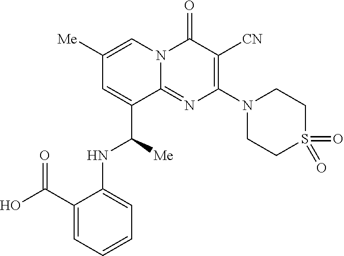 [SMILES: Cc1cc([C@@H](C)Nc2ccccc2C(=O)O)c2nc(N3CCS(=O)(=O)CC3)c(C#N)c(=O)n2c1] (R)-2-((1-(3-cyano-2-(1,1- dioxidothiomorpholino)-7-methyl-4-oxo-4H- pyrido[1,2-a]pyrimidin-9-yl)ethyl)amino)benzoic acid | LCMS [M + 1]⁺ = 482.2 |
| 10-69 | 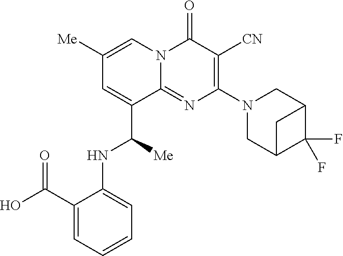 [SMILES: Cc1cc([C@@H](C)Nc2ccccc2C(=O)O)c2nc(N3CC4CC(C3)C4(F)F)c(C#N)c(=O)n2c1] 2-(((1R)-1-(3-cyano-2-(6,6-difluoro-3- azabicyclo[3.1.1]heptan-3-yl)-7-methyl-4-oxo- 4H-pyrido[1,2-a]pyrimidin-9- yl)ethyl)amino)benzoic acid | LCMS [M + 1]⁺ = 480.2 |
| 10-70 | 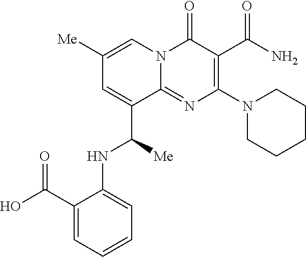 [SMILES: Cc1cc([C@@H](C)Nc2ccccc2C(=O)O)c2nc(N3CCCCC3)c(C(N)=O)c(=O)n2c1] (R)-2-((1-(3-carbamoyl-7-methyl-4-oxo-2- (piperidin-1-yl)-4H-pyrido[1,2-a]pyrimidin-9- yl)ethyl)amino)benzoic acid | LCMS [M + 1]⁺ = 450.3 |
| 10-71 | 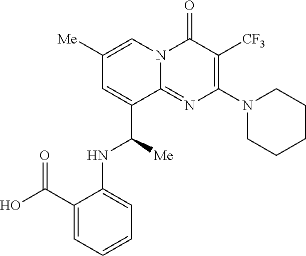 [SMILES: Cc1cc([C@@H](C)Nc2ccccc2C(=O)O)c2nc(N3CCCCC3)c(C(F)(F)F)c(=O)n2c1] (R)-2-((1-(7-methyl-4-oxo-2-(piperidin-1-yl)-3- (trifluoromethyl)-4H-pyrido[1,2-a]pyrimidin-9- yl)ethyl)amino)benzoic acid | LCMS [M + 1]⁺ = 475.2 |
| 10-72 | 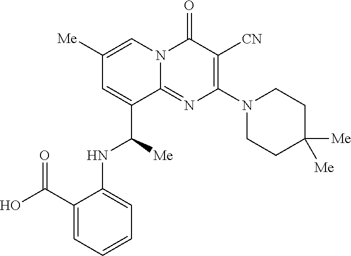 [SMILES: Cc1cc([C@@H](C)Nc2ccccc2C(=O)O)c2nc(N3CCC(C)(C)CC3)c(C#N)c(=O)n2c1] (R)-2-((1-(3-cyano-2-(4,4-dimethylpiperidin-1-yl)- 7-methyl-4-oxo-4H-pyrido[1,2-a]pyrimidin-9- yl)ethyl)amino)benzoic acid | LCMS [M + 1]⁺ = 460.3 |
| 10-73 | 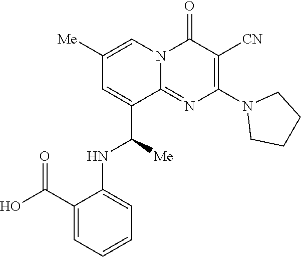 [SMILES: Cc1cc([C@@H](C)Nc2ccccc2C(=O)O)c2nc(N3CCCC3)c(C#N)c(=O)n2c1] (R)-2-((1-(3-cyano-7-methyl-4-oxo-2-(pyrrolidin- 1-yl)-4H-pyrido[1,2-a]pyrimidin-9- yl)ethyl)amino)benzoic acid | LCMS [M + 1]⁺ = 418.1 |
| 10-74 | 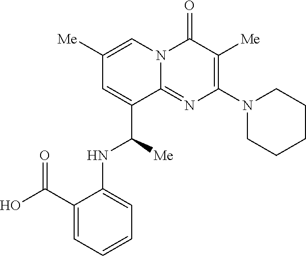 [SMILES: Cc1cc([C@@H](C)Nc2ccccc2C(=O)O)c2nc(N3CCCCC3)c(C)c(=O)n2c1] (R)-2-((1-(3,7-dimethyl-4-oxo-2-(piperidin-1-yl)- 4H-pyrido[1,2-a]pyrimidin-9- yl)ethyl)amino)benzoic acid | LCMS [M + 1]⁺ = 421.2 |
| 10-75 | 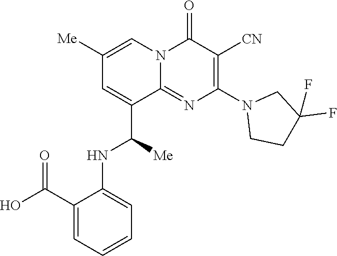 [SMILES: Cc1cc([C@@H](C)Nc2ccccc2C(=O)O)c2nc(N3CCC(F)(F)C3)c(C#N)c(=O)n2c1] (R)-2-((1-(3-cyano-2-(3,3-difluoropyrrolidin-1-yl)- 7-methyl-4-oxo-4H-pyrido[1,2-a]pyrimidin-9- yl)ethyl)amino)benzoic acid | LCMS [M + 1]⁺ = 454.2 |
| 10-76 | 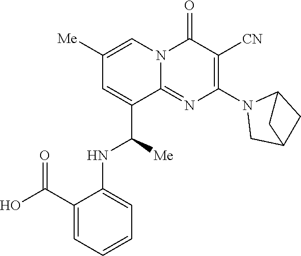 [SMILES: Cc1cc([C@@H](C)Nc2ccccc2C(=O)O)c2nc(N3CC4CC3C4)c(C#N)c(=O)n2c1] (R)-2-((1-(2-(2-azabicyclo[2.1.1]hexan-2-yl)-3- cyano-7-methyl-4-oxo-4H-pyrido[1,2-a]pyrimidin- 9-yl)ethyl)amino)benzoic acid | LCMS [M + 1]⁺ = 430.1 |
| 10-77 | 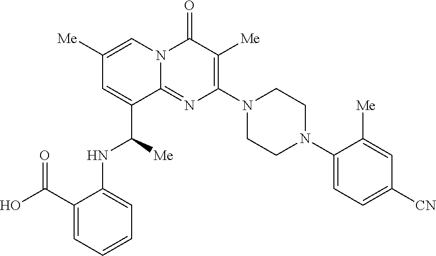 [SMILES: Cc1cc([C@@H](C)Nc2ccccc2C(=O)O)c2nc(N3CCN(c4ccc(C#N)cc4C)CC3)c(C)c(=O)n2c1] (R)-2-((1-(2-(4-(4-cyano-2- methylphenyl)piperazin-1-yl)-3,7-dimethyl-4-oxo- 4H-pyrido[1,2-a]pyrimidin-9- yl)ethyl)amino)benzoic acid | LCMS [M + 1]⁺ = 537.4 |
| 10-78 | 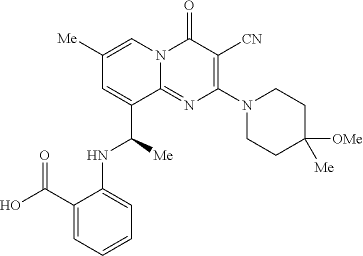 [SMILES: COC1(C)CCN(c2nc3c([C@@H](C)Nc4ccccc4C(=O)O)cc(C)cn3c(=O)c2C#N)CC1] (R)-2-((1-(3-cyano-2-(4-methoxy-4- methylpiperidin-1-yl)-7-methyl-4-oxo-4H- pyrido[1,2-a]pyrimidin-9-yl)ethyl)amino)benzoic acid | LCMS [M + 1]⁺ = 476.3 |
| 10-79 | 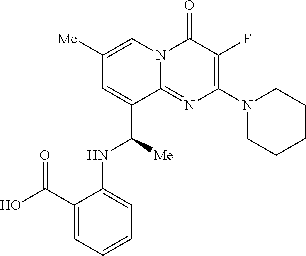 [SMILES: Cc1cc([C@@H](C)Nc2ccccc2C(=O)O)c2nc(N3CCCCC3)c(F)c(=O)n2c1] (R)-2-((1-(3-fluoro-7-methyl-4-oxo-2-(piperidin-1- yl)-4H-pyrido[1,2-a]pyrimidin-9- yl)ethyl)amino)benzoic acid | LCMS [M + 1]⁺ = 425.2 |
| 10-80 | 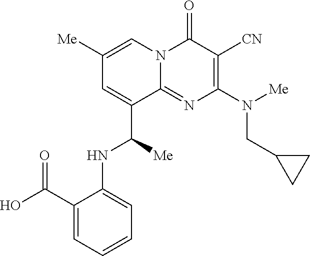 [SMILES: Cc1cc([C@@H](C)Nc2ccccc2C(=O)O)c2nc(N(C)CC3CC3)c(C#N)c(=O)n2c1] (R)-2-((1-(3-cyano-2- ((cyclopropylmethyl)(methyl)amino)-7-methyl-4- oxo-4H-pyrido[1,2-a]pyrimidin-9- yl)ethyl)amino)benzoic acid | LCMS [M + 1]⁺ = 432.1 |
| 10-81 | 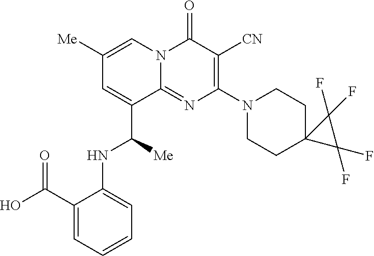 [SMILES: Cc1cc([C@@H](C)Nc2ccccc2C(=O)O)c2nc(N3CCC4(CC3)C(F)(F)C4(F)F)c(C#N)c(=O)n2c1] (R)-2-((1-(3-cyano-7-methyl-4-oxo-2-(1,1,2,2- tetrafluoro-6-azaspiro[2.5]octan-6-yl)-4H- pyrido[1,2-a]pyrimidin-9-yl)ethyl)amino)benzoic acid | LCMS [M + 1]⁺ = 530.2 |
| 10-82 | 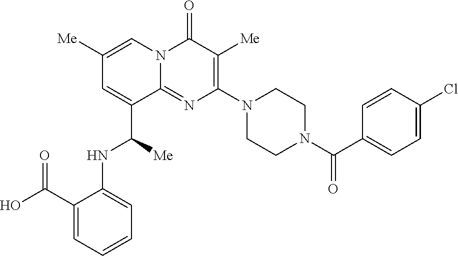 [SMILES: Cc1cc([C@@H](C)Nc2ccccc2C(=O)O)c2nc(N3CCN(C(=O)c4ccc(Cl)cc4)CC3)c(C)c(=O)n2c1] (R)-2-((1-(2-(4-(4-chlorobenzoyl)piperazin-1-yl)- 3,7-dimethyl-4-oxo-4H-pyrido[1,2-a]pyrimidin-9- yl)ethyl)amino)benzoic acid | LCMS [M + 1]⁺ = 560.1 |
| 10-83 | 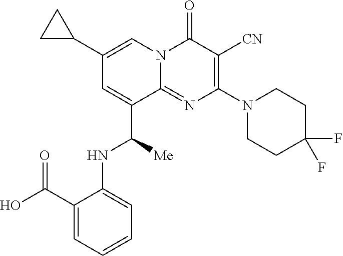 [SMILES: C[C@@H](Nc1ccccc1C(=O)O)c1cc(C2CC2)cn2c(=O)c(C#N)c(N3CCC(F)(F)CC3)nc12] (R)-2-((1-(3-cyano-7-cyclopropyl-2-(4,4- difluoropiperidin-1-yl)-4-oxo-4H-pyrido[1,2- a]pyrimidin-9-yl) ethyl)amino)benzoic acid | LCMS [M + 1]⁺ = 494.2 |
| 10-84 | 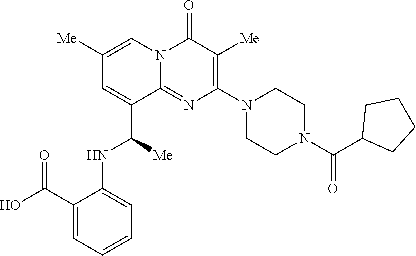 [SMILES: Cc1cc([C@@H](C)Nc2ccccc2C(=O)O)c2nc(N3CCN(C(=O)C4CCCC4)CC3)c(C)c(=O)n2c1] (R)-2-((1-(2-(4-(cyclopentanecarbonyl)piperazin- 1-yl)-3,7-dimethyl-4-oxo-4H-pyrido[1,2- a]pyrimidin-9-yl)ethyl)amino)benzoic acid | LCMS [M + 1]⁺ = 518.2 |
| 10-85 | 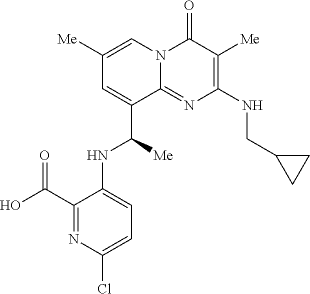 [SMILES: Cc1cc([C@@H](C)Nc2ccc(Cl)nc2C(=O)O)c2nc(NCC3CC3)c(C)c(=O)n2c1] (R)-6-chloro-3-((1-(2-((cyclopropylmethyl)amino)- 3,7-dimethyl-4-oxo-4H-pyrido[1,2-a]pyrimidin-9- yl)ethyl)amino)picolinic acid | LCMS [M + 1]⁺ = 442.1 |
| 10-86 | 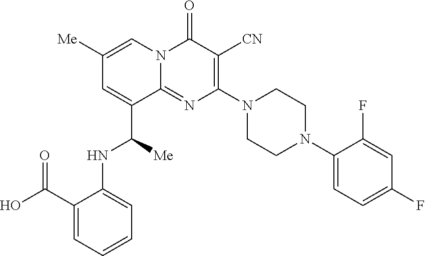 [SMILES: Cc1cc([C@@H](C)Nc2ccccc2C(=O)O)c2nc(N3CCN(c4ccc(F)cc4F)CC3)c(C#N)c(=O)n2c1] (R)-2-((1-(3-cyano-2-(4-(2,4- difluorophenyl)piperazin-1-yl)-7-methyl-4-oxo- 4H-pyrido[1,2-a]pyrimidin-9- yl)ethyl)amino)benzoic acid | LCMS [M + 1]⁺ = 545.5 |
| 10-87 | 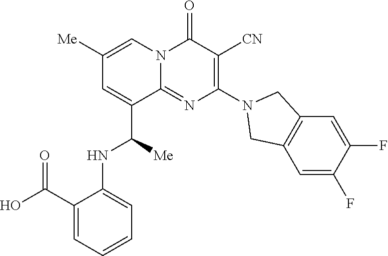 [SMILES: Cc1cc([C@@H](C)Nc2ccccc2C(=O)O)c2nc(N3Cc4cc(F)c(F)cc4C3)c(C#N)c(=O)n2c1] (R)-2-((1-(3-cyano-2-(5,6-difluoroisoindolin-2-yl)- 7-methyl-4-oxo-4H-pyrido[1,2-a]pyrimidin-9- yl)ethyl)amino)benzoic acid | LCMS [M + 1]⁺ = 502.3 |
| 10-88 | 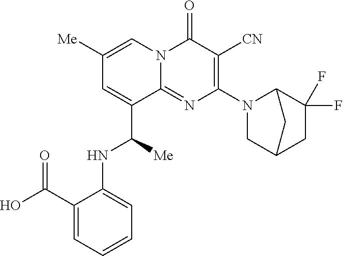 [SMILES: Cc1cc([C@@H](C)Nc2ccccc2C(=O)O)c2nc(N3CC4CC3C(F)(F)C4)c(C#N)c(=O)n2c1] 2-(((1R)-1-(3-cyano-2-(6,6-difluoro-2- azabicyclo[2.2.1]heptan-2-yl)-7-methyl-4-oxo- 4H-pyrido[1,2-a]pyrimidin-9- yl)ethyl)amino)benzoic acid | LCMS [M + 1]⁺ = 480.3 |
| 10-89 | 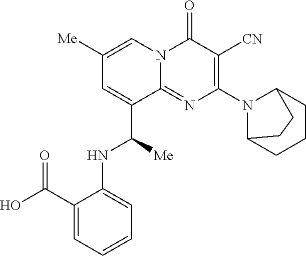 [SMILES: Cc1cc([C@@H](C)Nc2ccccc2C(=O)O)c2nc(N3C4CCCC3CC4)c(C#N)c(=O)n2c1] 2-(((1R)-1-(2-(8-azabicyclo[3.2.1]octan-8-yl)-3- cyano-7-methyl-4-oxo-4H-pyrido[1,2-a]pyrimidin- 9-yl)ethyl)amino)benzoic acid | LCMS [M + 1]⁺ = 494.3 |
| 10-90 | 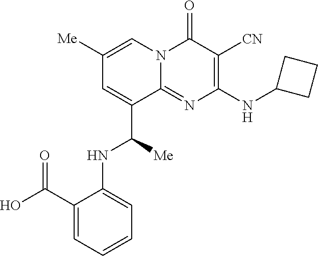 [SMILES: Cc1cc([C@@H](C)Nc2ccccc2C(=O)O)c2nc(NC3CCC3)c(C#N)c(=O)n2c1] (R)-2-((1-(3-cyano-2-(cyclobutylamino)-7-methyl- 4-oxo-4H-pyrido[1,2-a]pyrimidin-9- yl)ethyl)amino)benzoic acid | LCMS [M + 1]⁺ = 418.2 |
| 10-91 | 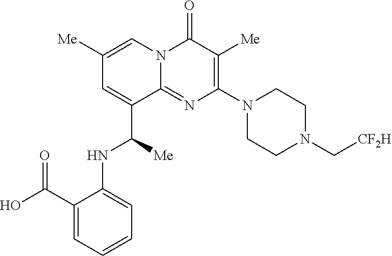 [SMILES: Cc1cc([C@@H](C)Nc2ccccc2C(=O)O)c2nc(N3CCN(CC(F)F)CC3)c(C)c(=O)n2c1] (R)-2-((1-(2-(4-(2,2-difluoroethyl)piperazin-1-yl)- 3,7-dimethyl-4-oxo-4H-pyrido[1,2-a]pyrimidin-9- yl)ethyl)amino)benzoic acid | LCMS [M + 1]⁺ = 486.2 |
| 10-92 | 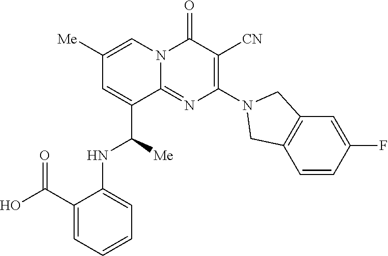 [SMILES: Cc1cc([C@@H](C)Nc2ccccc2C(=O)O)c2nc(N3Cc4ccc(F)cc4C3)c(C#N)c(=O)n2c1] (R)-2-((1-(3-cyano-2-(5-fluoroisoindolin-2-yl)-7- methyl-4-oxo-4H-pyrido[1,2-a]pyrimidin-9- yl)ethyl)amino)benzoic acid | LCMS [M + 1]⁺ = 484.4 |
| 10-93 | 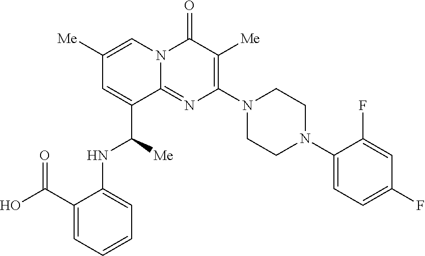 [SMILES: Cc1cc([C@@H](C)Nc2ccccc2C(=O)O)c2nc(N3CCN(c4ccc(F)cc4F)CC3)c(C)c(=O)n2c1] (R)-2-((1-(2-(4-(2,4-difluorophenyl)piperazin-1- yl)-3,7-dimethyl-4-oxo-4H-pyrido[1,2-a]pyrimidin- 9-yl)ethyl)amino)benzoic acid | LCMS [M + 1]⁺ = 534.31 |
| 10-94 | 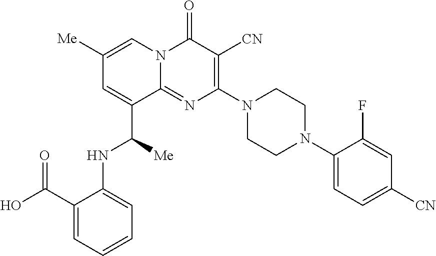 [SMILES: Cc1cc([C@@H](C)Nc2ccccc2C(=O)O)c2nc(N3CCN(c4ccc(C#N)cc4F)CC3)c(C#N)c(=O)n2c1] (R)-2-((1-(3-cyano-2-(4-(4-cyano-2- fluorophenyl)piperazin-1-yl)-7-methyl-4-oxo-4H- pyrido[1,2-a]pyrimidin-9-yl)ethyl)amino)benzoic acid | LCMS [M + 1]⁺ = 552.3 |
| 10-95 And 10-96 | 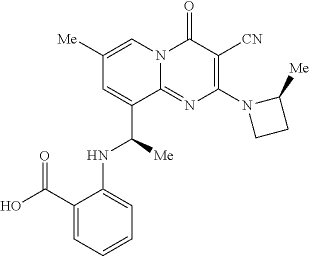 [SMILES: Cc1cc([C@@H](C)Nc2ccccc2C(=O)O)c2nc(N3CC[C@@H]3C)c(C#N)c(=O)n2c1] 2-(((R)-1-(3-cyano-7-methyl-2-((S)-2- methylazetidin-1-yl)-4-oxo-4H-pyrido[1,2- a]pyrimidin-9-yl)ethyl)amino)benzoic acid AND 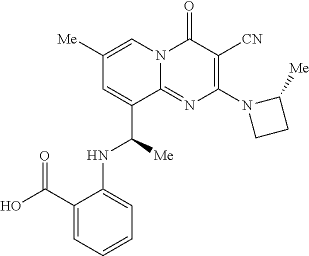 [SMILES: Cc1cc([C@@H](C)Nc2ccccc2C(=O)O)c2nc(N3CC[C@H]3C)c(C#N)c(=O)n2c1] 2-(((R)-1-(3-cyano-7-methyl-2-((R)-2- methylazetidin-1-yl)-4-oxo-4H-pyrido[1,2- a]pyrimidin-9-yl)ethyl)amino)benzoic acid | First eluting isomer after saponification: LCMS [M + 1]⁺ = 418.3 Second eluting isomer after saponification: LCMS [M + 1]⁺ = 418.3 |
| 10-97 | 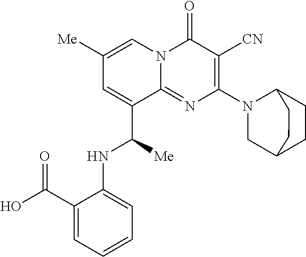 [SMILES: Cc1cc([C@@H](C)Nc2ccccc2C(=O)O)c2nc(N3CC4CCC3CC4)c(C#N)c(=O)n2c1] (R)-2-((1-(2-(2-azabicyclo[2.2.2]octan-2-yl)-3- cyano-7-methyl-4-oxo-4H-pyrido[1,2-a]pyrimidin- 9-yl)ethyl)amino)benzoic acid | LCMS [M + 1]⁺ = 458.2 |
| 10-98 | 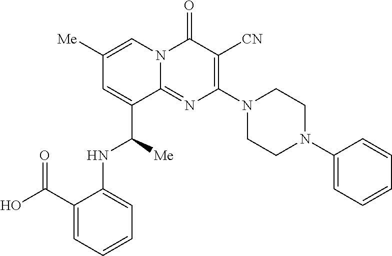 [SMILES: Cc1cc([C@@H](C)Nc2ccccc2C(=O)O)c2nc(N3CCN(c4ccccc4)CC3)c(C#N)c(=O)n2c1] (R)-2-((1-(3-cyano-7-methyl-4-oxo-2-(4- phenylpiperazin-1-yl)-4H-pyrido[1,2-a]pyrimidin- 9-yl)ethyl)amino)benzoic acid | LCMS [M + 1]⁺ = 509.4 |
| 10-99 | 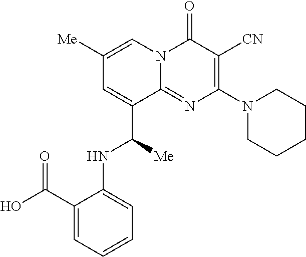 [SMILES: Cc1cc([C@@H](C)Nc2ccccc2C(=O)O)c2nc(N3CCCCC3)c(C#N)c(=O)n2c1] (R)-2-((1-(3-cyano-7-methyl-4-oxo-2-(piperidin-1- yl)-4H-pyrido[1,2-a]pyrimidin-9- yl)ethyl)amino)benzoic acid | HRMS: Calculated for C₂₄H₂₅N₅O₃ [M + 1]⁺: 432.2030; found 432.2038. [α]_{D}²⁵ −338.825 (c = 0.753 g, DMSO) |
| 10-100 | 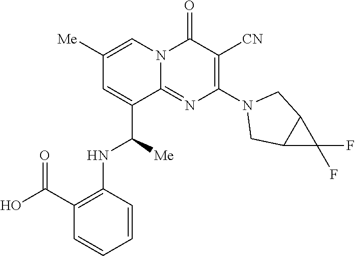 [SMILES: Cc1cc([C@@H](C)Nc2ccccc2C(=O)O)c2nc(N3CC4C(C3)C4(F)F)c(C#N)c(=O)n2c1] 2-(((1R)-1-(3-cyano-2-(6,6-difluoro-3- azabicyclo[3.1.0]hexan-3-yl)-7-methyl-4-oxo-4H- pyrido[1,2-a]pyrimidin-9-yl)ethyl)amino)benzoic acid | LCMS [M + 1]⁺ = 466.3 |
| 10-101 | 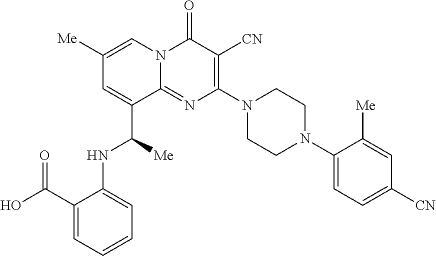 [SMILES: Cc1cc([C@@H](C)Nc2ccccc2C(=O)O)c2nc(N3CCN(c4ccc(C#N)cc4C)CC3)c(C#N)c(=O)n2c1] (R)-2-((1-(3-cyano-2-(4-(4-cyano-2- methylphenyl)piperazin-1-yl)-7-methyl-4-oxo-4H- pyrido[1,2-a]pyrimidin-9-yl)ethyl)amino)benzoic acid | LCMS [M + 1]⁺ = 548.2 |
| 10-102 | 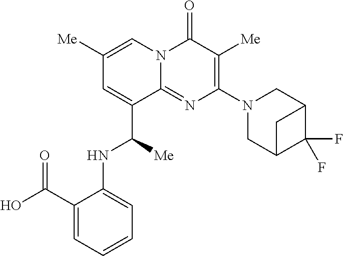 [SMILES: Cc1cc([C@@H](C)Nc2ccccc2C(=O)O)c2nc(N3CC4CC(C3)C4(F)F)c(C)c(=O)n2c1] 2-(((1R)-1-(2-(6,6-difluoro-3- azabicyclo[3.1.1]heptan-3-yl)-3,7-dimethyl-4- oxo-4H-pyrido[1,2-a]pyrimidin-9- yl)ethyl)amino)benzoic acid | LCMS [M + 1]⁺ = 469.3 |
| 10-103 | 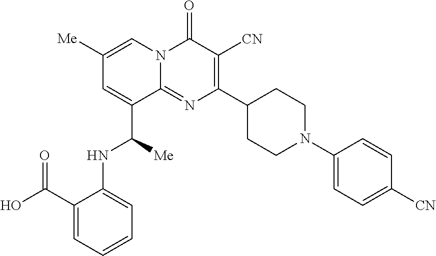 [SMILES: Cc1cc([C@@H](C)Nc2ccccc2C(=O)O)c2nc(C3CCN(c4ccc(C#N)cc4)CC3)c(C#N)c(=O)n2c1] (R)-2-((1-(3-cyano-2-(1-(4- cyanophenyl) piperidin-4-yl)-7-methyl-4-oxo-4H- pyrido[1,2-a]pyrimidin-9-yl)ethyl)amino)benzoic acid | LCMS [M + 1]⁺ = 533.3 |
| 10-104 | 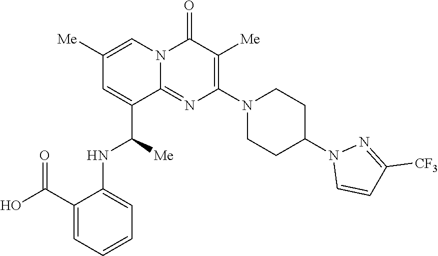 [SMILES: Cc1cc([C@@H](C)Nc2ccccc2C(=O)O)c2nc(N3CCC(n4ccc(C(F)(F)F)n4)CC3)c(C)c(=O)n2c1] (R)-2-((1-(3,7-dimethyl-4-oxo-2-(4-(3- (trifluoromethyl)-1H-pyrazol-1-yl)piperidin-1-yl)- 4H-pyrido[1,2-a]pyrimidin-9- yl)ethyl)amino)benzoic acid | ¹H NMR (400 MHz, CD₃OD) δ (ppm) = 8.65 (s, 1H), 7.90 (d, J = 7.6 Hz, 1H), 7.84 (s, 1H), 7.64 (s, 1H), 7.16 (t, J = 7.8 Hz, 1H), 6.58 (s, 1H), 6.54 (t, J = 7.4 Hz, 1H), 6.36 (d, J = 8.6 Hz, 1H), 5.44 (q, J = 6.9 Hz, 1H), 4.60-4.49 (m, 1H), 4.25- 4.15 (m, 2H), 3.28-3.16 (m, 2H), 2.31 (s, 3H), 2.30-2.17 (m, 7H), 1.66 (d, J = 6.6 Hz, 3H). LCMS [M + 1]⁺ = 555.2 |
| 10-105 | 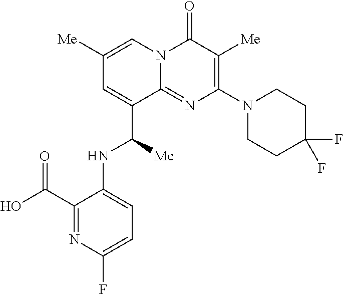 [SMILES: Cc1cc([C@@H](C)Nc2ccc(F)nc2C(=O)O)c2nc(N3CCC(F)(F)CC3)c(C)c(=O)n2c1] (R)-3-((1-(2-(4,4-difluoropiperidin-1-yl)-3,7- dimethyl-4-oxo-4H-pyrido[1,2-a]pyrimidin-9- yl)ethyl)amino)-6-fluoropicolinic acid | |
| 10-106 | 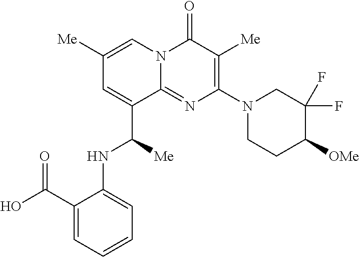 [SMILES: CO[C@H]1CCN(c2nc3c([C@@H](C)Nc4ccccc4C(=O)O)cc(C)cn3c(=O)c2C)CC1(F)F] 2-(((R)-1-(2-((S)-3,3-difluoro-4-methoxypiperidin- 1-yl)-3,7-dimethyl-4-oxo-4H-pyrido[1,2- a]pyrimidin-9-yl)ethyl)amino)benzoic acid OR 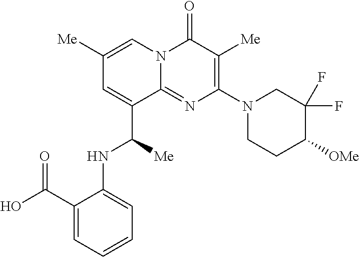 [SMILES: CO[C@@H]1CCN(c2nc3c([C@@H](C)Nc4ccccc4C(=O)O)cc(C)cn3c(=O)c2C)CC1(F)F] 2-(((R)-1-(2-((R)-3,3-difluoro-4-methoxypiperidin- 1-yl)-3,7-dimethyl-4-oxo-4H-pyrido[1,2- a]pyrimidin-9-yl)ethyl)amino)benzoic acid | LCMS [M + 1]⁺ = 487.2 |
| 10-107 | 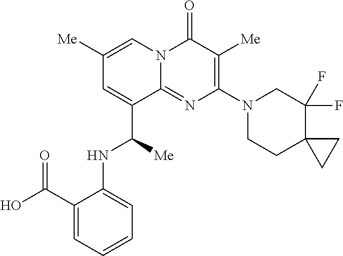 [SMILES: Cc1cc([C@@H](C)Nc2ccccc2C(=O)O)c2nc(N3CCC4(CC4)C(F)(F)C3)c(C)c(=O)n2c1] (R)-2-((1-(2-(4,4-difluoro-6-azaspiro[2.5]octan-6- yl)-3, R 7-dimethyl-4-oxo-4H-pyrido[1,2- a]pyrimidin-9-yl) ethyl)amino)benzoic acid | LCMS [M + 1]⁺ = 483.1 |
| 10-108 | 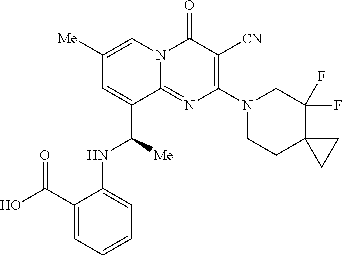 [SMILES: Cc1cc([C@@H](C)Nc2ccccc2C(=O)O)c2nc(N3CCC4(CC4)C(F)(F)C3)c(C#N)c(=O)n2c1] (R)-2-((1-(3-cyano-2-(4,4-difluoro-6- azaspiro[2.5]octan-6-yl)-7-methyl-4-oxo-4H- pyrido[1,2-a]pyrimidin-9-yl)ethyl)amino)benzoic acid | LCMS [M + 1]⁺ = 494.3 |
| 10-109 | 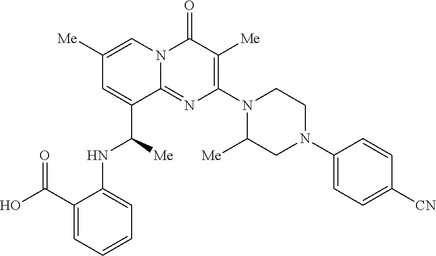 [SMILES: Cc1cc([C@@H](C)Nc2ccccc2C(=O)O)c2nc(N3CCN(c4ccc(C#N)cc4)CC3C)c(C)c(=O)n2c1] 2-(((1R)-1-(3-cyano-2-(4-(4-cyanophenyl)-2- methylpiperazin-1-yl)-7-methyl-4-oxo-4H- pyrido[1,2-a]pyrimidin-9-yl)ethyl)amino)benzoic acid | LCMS [M + 1]⁺ = 548.3 |
| 10-110 | 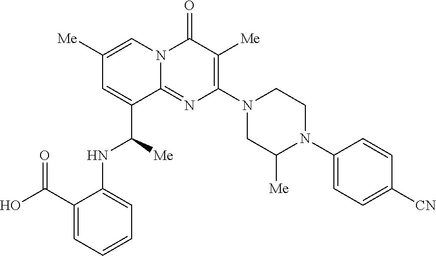 [SMILES: Cc1cc([C@@H](C)Nc2ccccc2C(=O)O)c2nc(N3CCN(c4ccc(C#N)cc4)C(C)C3)c(C)c(=O)n2c1] 2-(((1R)-1-(2-(4-(4-cyanophenyl)-3- methylpiperazin-1-yl)-3,7-dimethyl-4-oxo-4H- pyrido[1,2-a]pyrimidin-9-yl)ethyl)amino)benzoic acid | LCMS [M + 1]⁺ = 537.3 |
| 10-111 | 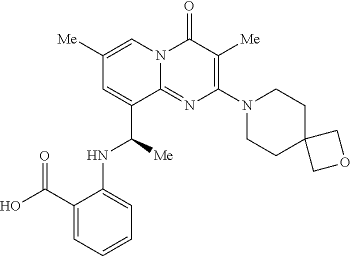 [SMILES: Cc1cc([C@@H](C)Nc2ccccc2C(=O)O)c2nc(N3CCC4(CC3)COC4)c(C)c(=O)n2c1] (R)-2-((1-(3,7-dimethyl-4-oxo-2-(2-oxa-7- azaspiro[3.5]nonan-7-yl)-4H-pyrido[1,2- a]pyrimidin-9-yl) ethyl)amino)benzoic acid | LCMS [M + 1]⁺ = 463.3 |
| 10-112 | 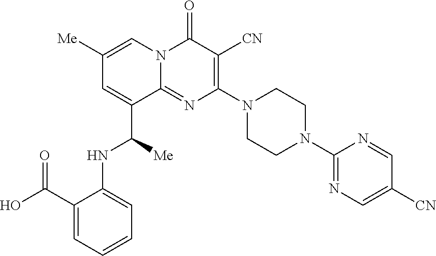 [SMILES: Cc1cc([C@@H](C)Nc2ccccc2C(=O)O)c2nc(N3CCN(c4ncc(C#N)cn4)CC3)c(C#N)c(=O)n2c1] (R)-2-((1-(3-cyano-2-(4-(5-cyanopyrimidin-2- yl)piperazin-1-yl)-7-methyl-4-oxo-4H-pyrido[1,2- a]pyrimidin-9-yl)ethyl)amino)benzoic acid | LCMS [M + 1]⁺ = 536.3 |
| 10-113 | 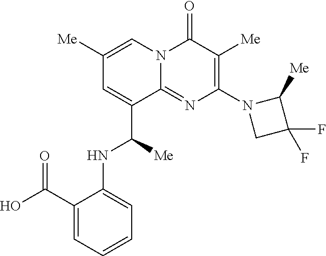 [SMILES: Cc1cc([C@@H](C)Nc2ccccc2C(=O)O)c2nc(N3CC(F)(F)[C@@H]3C)c(C)c(=O)n2c1] 2-(((R)-1-(2-((S)-3,3-difluoro-2-methylazetidin-1- yl)-3,7-dimethyl-4-oxo-4H-pyrido[1,2-a]pyrimidin- 9-yl)ethyl)amino)benzoic acid OR 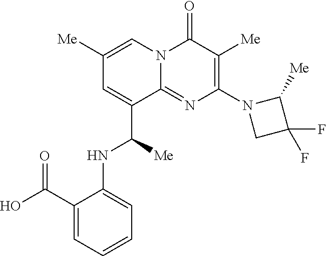 [SMILES: Cc1cc([C@@H](C)Nc2ccccc2C(=O)O)c2nc(N3CC(F)(F)[C@H]3C)c(C)c(=O)n2c1] 2-(((R)-1-(2-((R)-3,3-difluoro-2-methylazetidin-1- yl)-3,7-dimethyl-4-oxo-4H-pyrido[1,2-a]pyrimidin- 9-yl)ethyl)amino)benzoic acid | LCMS [M + Na]⁺ = 465.0 |
| 10-114 | 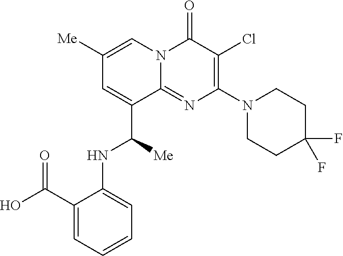 [SMILES: Cc1cc([C@@H](C)Nc2ccccc2C(=O)O)c2nc(N3CCC(F)(F)CC3)c(Cl)c(=O)n2c1] (R)-2-((1-(3-chloro-2-(4,4-difluoropiperidin-1-yl)- 7-methyl-4-oxo-4H-pyrido[1,2-a]pyrimidin-9- yl)ethyl)amino)benzoic acid | LCMS [M + 1]⁺ = 477.2 |
| 10-115 | 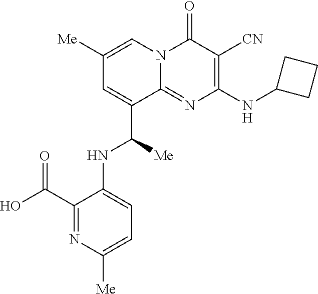 [SMILES: Cc1cc([C@@H](C)Nc2ccc(C)nc2C(=O)O)c2nc(NC3CCC3)c(C#N)c(=O)n2c1] (R)-3-((1-(3-cyano-2-(cyclobutylamino)-7-methyl- 4-oxo-4H-pyrido[1,2-a]pyrimidin-9- yl)ethyl)amino)-6-methylpicolinic acid | LCMS [M + 1]⁺ = 433.2 |
| 10-116 | 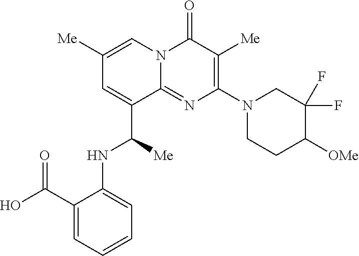 [SMILES: COC1CCN(c2nc3c([C@@H](C)Nc4ccccc4C(=O)O)cc(C)cn3c(=O)c2C)CC1(F)F] 2-(((1R)-1-(2-(3,3-difluoro-4-methoxypiperidin-1- yl)-3,7-dimethyl-4-oxo-4H-pyrido[1,2-a]pyrimidin- 9-yl)ethyl)amino)benzoic acid | LCMS [M + 1]⁺ = 487.2 |
| 10-117 | 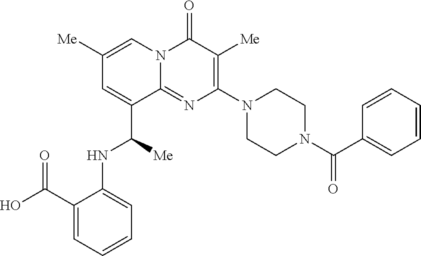 [SMILES: Cc1cc([C@@H](C)Nc2ccccc2C(=O)O)c2nc(N3CCN(C(=O)c4ccccc4)CC3)c(C)c(=O)n2c1] (R)-2-((1-(2-(4-benzoylpiperazin-1-yl)-3,7- dimethyl-4-oxo-4H-pyrido[1,2-a]pyrimidin-9- yl)ethyl)amino)benzoic acid | LCMS [M + 1]⁺ = 526.4 |
| 10-118 | 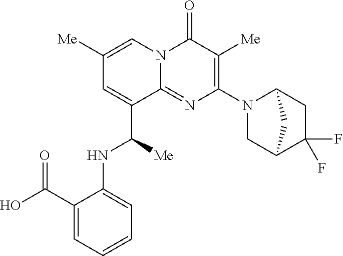 [SMILES: Cc1cc([C@@H](C)Nc2ccccc2C(=O)O)c2nc(N3C[C@H]4C[C@@H]3CC4(F)F)c(C)c(=O)n2c1] 2-(((R)-1-(2-((1R,4R)-5,5-difluoro-2- azabicyclo[2.2.1]heptan-2-yl)-3,7-dimethyl-4- oxo-4H-pyrido[1,2-a]pyrimidin-9- yl)ethyl)amino)benzoic acid OR 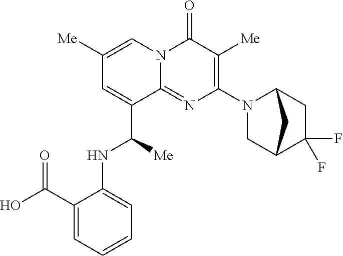 [SMILES: Cc1cc([C@@H](C)Nc2ccccc2C(=O)O)c2nc(N3C[C@@H]4C[C@H]3CC4(F)F)c(C)c(=O)n2c1] 2-(((R)-1-(2-((1S,4S)-5,5-difluoro-2- azabicyclo[2.2.1]heptan-2-yl)-3,7-dimethyl-4- oxo-4H-pyrido[1,2-a]pyrimidin-9- yl)ethyl)amino)benzoic acid | Second eluting isomer after saponification (SFC purification): LCMS [M + 1]⁺ = 469.3 |
| 10-119 | 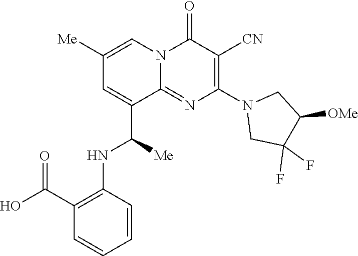 [SMILES: CO[C@@H]1CN(c2nc3c([C@@H](C)Nc4ccccc4C(=O)O)cc(C)cn3c(=O)c2C#N)CC1(F)F] 2-(((R)-1-(3-cyano-2-((R)-3,3-difluoro-4- methoxypyrrolidin-1-yl)-7-methyl-4-oxo-4H- pyrido[1,2-a]pyrimidin-9-yl)ethyl)amino)benzoic acid OR 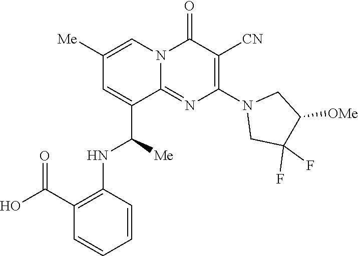 [SMILES: CO[C@H]1CN(c2nc3c([C@@H](C)Nc4ccccc4C(=O)O)cc(C)cn3c(=O)c2C#N)CC1(F)F] 2-(((R)-1-(3-cyano-2-((S)-3,3-difluoro-4- methoxypyrrolidin-1-yl)-7-methyl-4-oxo-4H- pyrido[1,2-a]pyrimidin-9-yl)ethyl)amino)benzoic acid | LCMS [M + 1]⁺ = 484.1 |
| 10-120 | 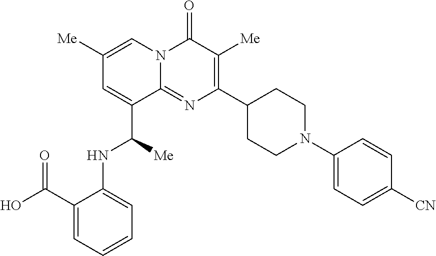 [SMILES: Cc1cc([C@@H](C)Nc2ccccc2C(=O)O)c2nc(C3CCN(c4ccc(C#N)cc4)CC3)c(C)c(=O)n2c1] (R)-2-((1-(2-(1-(4-cyanophenyl)piperidin-4-yl)- 3,7-dimethyl-4-oxo-4H-pyrido[1,2-a]pyrimidin-9- yl)ethyl)amino)benzoic acid | LCMS [M + 1]⁺ = 522.3 |
| 10-121 | 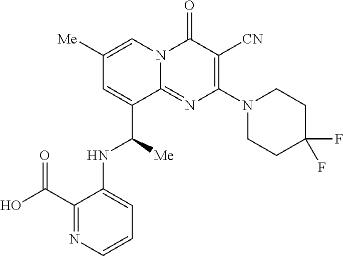 [SMILES: Cc1cc([C@@H](C)Nc2cccnc2C(=O)O)c2nc(N3CCC(F)(F)CC3)c(C#N)c(=O)n2c1] (R)-3-((1-(3-cyano-2-(4,4-difluoropiperidin-1-yl)- 7-methyl-4-oxo-4H-pyrido[1,2-a]pyrimidin-9- yl)ethyl)amino)picolinic acid | LCMS [M + 1]⁺ = 469.2 |
| 10-122 | 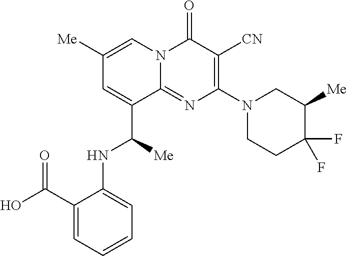 [SMILES: Cc1cc([C@@H](C)Nc2ccccc2C(=O)O)c2nc(N3CCC(F)(F)[C@H](C)C3)c(C#N)c(=O)n2c1] 2-(((R)-1-(3-cyano-2-((R)-4,4-difluoro-3- methylpiperidin-1-yl)-7-methyl-4-oxo-4H- pyrido[1,2-a]pyrimidin-9-yl)ethyl)amino)benzoic acid OR 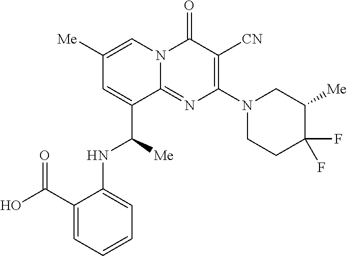 [SMILES: Cc1cc([C@@H](C)Nc2ccccc2C(=O)O)c2nc(N3CCC(F)(F)[C@@H](C)C3)c(C#N)c(=O)n2c1] 2-(((R)-1-(3-cyano-2-((S)-4,4-difluoro-3- methylpiperidin-1-yl)-7-methyl-4-oxo-4H- pyrido[1,2-a]pyrimidin-9-yl)ethyl)amino)benzoic acid | LCMS [M + 1]⁺ = 482.2 |
| 10-123 | 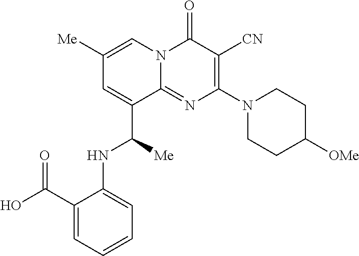 [SMILES: COC1CCN(c2nc3c([C@@H](C)Nc4ccccc4C(=O)O)cc(C)cn3c(=O)c2C#N)CC1] (R)-2-((1-(3-cyano-2-(4-methoxypiperidin-1-yl)-7- methyl-4-oxo-4H-pyrido[1,2-a]pyrimidin-9- yl)ethyl)amino)benzoic acid | LCMS [M + 1]⁺ = 462.3 |
| 10-124 | 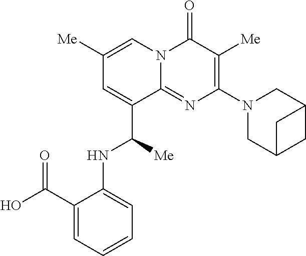 [SMILES: Cc1cc([C@@H](C)Nc2ccccc2C(=O)O)c2nc(N3CC4CC(C4)C3)c(C)c(=O)n2c1] (R)-2-((1-(2-(3-azabicyclo[3.1.1]heptan-3-yl)-3,7- dimethyl-4-oxo-4H-pyrido[1,2-a]pyrimidin-9- yl) ethyl)amino)benzoic acid | LCMS [M + 1]⁺ = 433.3 |
| 10-125 | 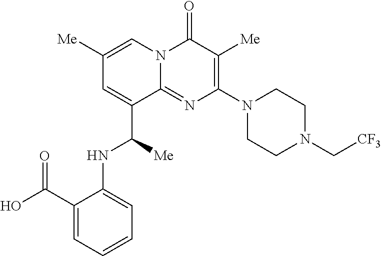 [SMILES: Cc1cc([C@@H](C)Nc2ccccc2C(=O)O)c2nc(N3CCN(CC(F)(F)F)CC3)c(C)c(=O)n2c1] (R)-2-((1-(3,7-dimethyl-4-oxo-2-(4-(2,2,2- trifluoroethyl)piperazin-1-yl)-4H-pyrido[1,2- a]pyrimidin-9-yl)ethyl)amino)benzoic acid | LCMS [M + 1]⁺ = 504.0 |
| 10-126 | 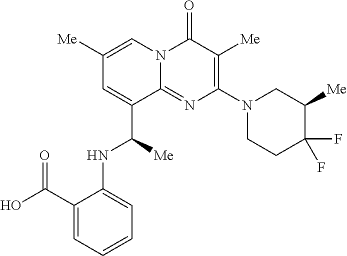 [SMILES: Cc1cc([C@@H](C)Nc2ccccc2C(=O)O)c2nc(N3CCC(F)(F)[C@H](C)C3)c(C)c(=O)n2c1] 2-(((R)-1-(2-((R)-4,4-difluoro-3-methylpiperidin-1- yl)-3,7-dimethyl-4-oxo-4H-pyrido[1,2-a]pyrimidin- 9-yl)ethyl)amino)benzoic acid | LCMS [M + 1]⁺ = 471.3 |
| 10-127 | 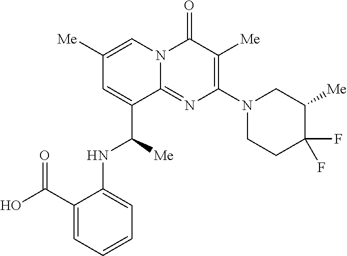 [SMILES: Cc1cc([C@@H](C)Nc2ccccc2C(=O)O)c2nc(N3CCC(F)(F)[C@@H](C)C3)c(C)c(=O)n2c1] 2-(((R)-1-(2-((S)-4,4-difluoro-3-methylpiperidin-1- yl)-3,7-dimethyl-4-oxo-4H-pyrido[1,2-a]pyrimidin- 9-yl)ethyl)amino)benzoic acid | LCMS [M + 1]⁺ = 471.2 |
| 10-128 | 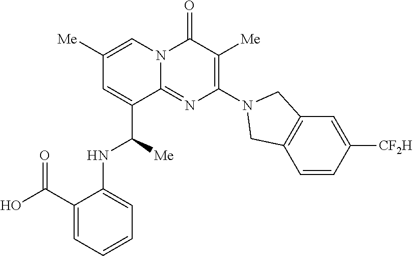 [SMILES: Cc1cc([C@@H](C)Nc2ccccc2C(=O)O)c2nc(N3Cc4ccc(C(F)F)cc4C3)c(C)c(=O)n2c1] (R)-2-((1-(2-(5-(difluoromethyl)isoindolin-2-yl)- 3,7-dimethyl-4-oxo-4H-pyrido[1,2-a]pyrimidin-9- yl)ethyl)amino)benzoic acid | LCMS [M + 1]⁺ = 505.2 |
| 10-129 | 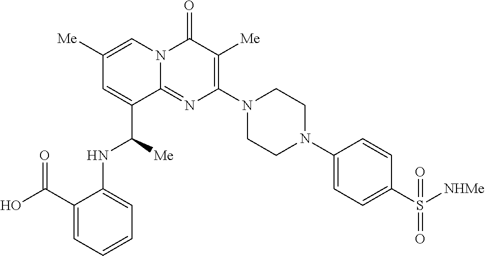 [SMILES: CNS(=O)(=O)c1ccc(N2CCN(c3nc4c([C@@H](C)Nc5ccccc5C(=O)O)cc(C)cn4c(=O)c3C)CC2)cc1] (R)-2-((1-(3,7-dimethyl-2-(4-(4-(N- methylsulfamoyl)phenyl)piperazin-1-yl)-4-oxo- 4H-pyrido[1,2-a]pyrimidin-9- yl)ethyl)amino)benzoic acid | LCMS [M + 1]⁺ = 591.0 |
| 10-130 | 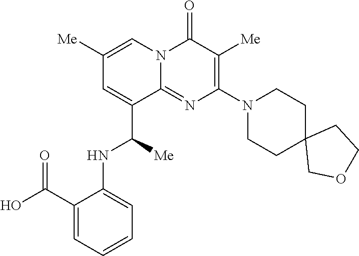 [SMILES: Cc1cc([C@@H](C)Nc2ccccc2C(=O)O)c2nc(N3CCC4(CCOC4)CC3)c(C)c(=O)n2c1] (R)-2-((1-(3,7-dimethyl-4-oxo-2-(2-oxa-8- azaspiro[4.5]decan-8-yl)-4H-pyrido[1,2- a]pyrimidin-9-yl) ethyl)amino)benzoic acid | LCMS [M + 1]⁺ = 477.2 |
| 10-131 | 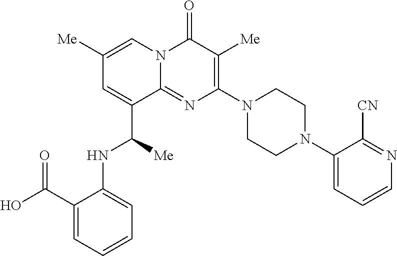 [SMILES: Cc1cc([C@@H](C)Nc2ccccc2C(=O)O)c2nc(N3CCN(c4cccnc4C#N)CC3)c(C)c(=O)n2c1] (R)-2-((1-(2-(4-(2-cyanopyridin-3-yl)piperazin-1- yl)-3,7-dimethyl-4-oxo-4H-pyrido[1,2-a]pyrimidin- 9-yl)ethyl)amino)benzoic acid | LCMS [M + 1]⁺ = 524.2 |
| 10-132 | 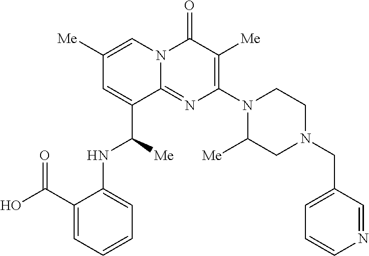 [SMILES: Cc1cc([C@@H](C)Nc2ccccc2C(=O)O)c2nc(N3CCN(Cc4cccnc4)CC3C)c(C)c(=O)n2c1] 2-(((1R)-1-(3,7-dimethyl-2-(2-methyl-4-(pyridin-3- ylmethyl)piperazin-1-yl)-4-oxo-4H-pyrido[1,2- a]pyrimidin-9-yl)ethyl)amino)benzoic acid | LCMS [M + 1]⁺ = 527.2 |
| 10-133 | 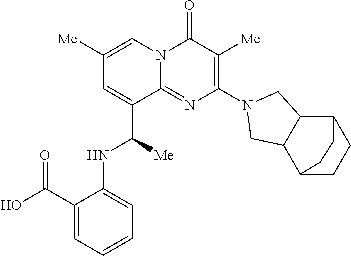 [SMILES: Cc1cc([C@@H](C)Nc2ccccc2C(=O)O)c2nc(N3CC4C5CCC(CC5)C4C3)c(C)c(=O)n2c1] 2-(((1R)-1-(3,7-dimethyl-2-(octahydro-2H-4,7- ethanoisoindol-2-yl)-4-oxo-4H-pyrido[1,2- a]pyrimidin-9-yl)ethyl)amino)benzoic acid | LCMS [M + 1]⁺ = 487.4 |
| 10-134 | 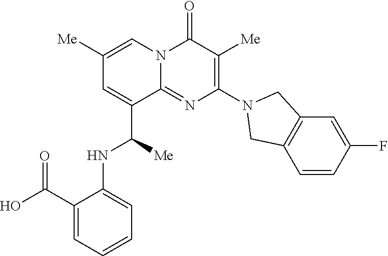 [SMILES: Cc1cc([C@@H](C)Nc2ccccc2C(=O)O)c2nc(N3Cc4ccc(F)cc4C3)c(C)c(=O)n2c1] (R)-2-((1-(2-(5-fluoroisoindolin-2-yl)-3,7-dimethyl- 4-oxo-4H-pyrido[1,2-a]pyrimidin-9- yl)ethyl)amino)benzoic acid | LCMS [M + 1]⁺ = 473.2 |
| 10-135 | 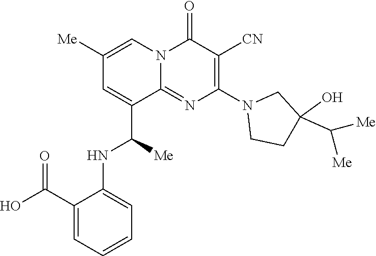 [SMILES: Cc1cc([C@@H](C)Nc2ccccc2C(=O)O)c2nc(N3CCC(O)(C(C)C)C3)c(C#N)c(=O)n2c1] 2-(((1R)-1-(2-(3-hydroxy-3-isopropylpyrrolidin-1- yl)-3,7-dimethyl-4-oxo-4H-pyrido[1,2-a]pyrimidin- 9-yl)ethyl)amino)benzoic acid | LCMS [M + 1]⁺ = 465.0 |
| 10-136 | 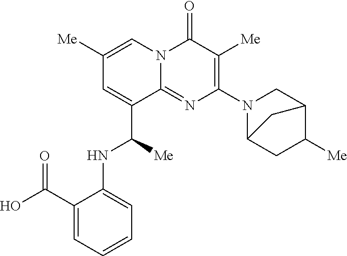 [SMILES: Cc1cc([C@@H](C)Nc2ccccc2C(=O)O)c2nc(N3CC4CC3CC4C)c(C)c(=O)n2c1] 2-(((1R)-1-(3,7-dimethyl-2-(5-methyl-2- azabicyclo[2.2.1]heptan-2-yl)-4-oxo-4H- pyrido[1,2-a]pyrimidin-9-yl)ethyl)amino)benzoic acid | LCMS [M + 1]⁺ = 447.0 |
| 10-137 | 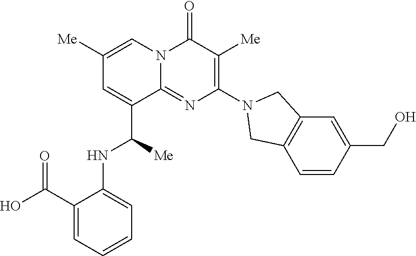 [SMILES: Cc1cc([C@@H](C)Nc2ccccc2C(=O)O)c2nc(N3Cc4ccc(CO)cc4C3)c(C)c(=O)n2c1] (R)-2-((1-(2-(5-(hydroxymethyl)isoindolin-2-yl)- 3,7-dimethyl-4-oxo-4H-pyrido[1,2-a]pyrimidin-9- yl)ethyl)amino)benzoic acid | LCMS [M + 1]⁺ = 485.0 |
| 10-138 | 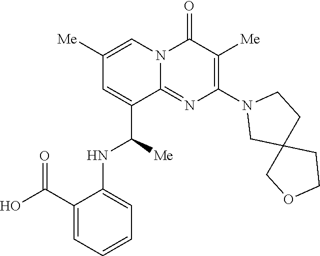 [SMILES: Cc1cc([C@@H](C)Nc2ccccc2C(=O)O)c2nc(N3CCC4(CCOC4)C3)c(C)c(=O)n2c1] 2-(((1R)-1-(3,7-dimethyl-4-oxo-2-(2-oxa-7- azaspiro[4.4]nonan-7-yl)-4H-pyrido[1,2- a]pyrimidin-9-yl)ethyl)amino)benzoic acid | LCMS [M + 1]⁺ = 463.2 |
| 10-139 | 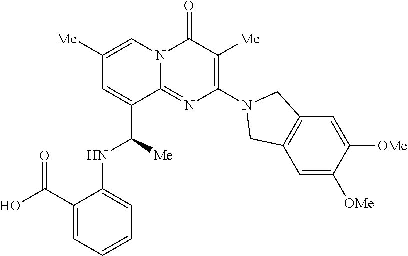 [SMILES: COc1cc2c(cc1OC)CN(c1nc3c([C@@H](C)Nc4ccccc4C(=O)O)cc(C)cn3c(=O)c1C)C2] (R)-2-((1-(2-(5,6-dimethoxyisoindolin-2-yl)-3,7- dimethyl-4-oxo-4H-pyrido[1,2-a]pyrimidin-9- yl)ethyl)amino)benzoic acid | LCMS [M + 1]⁺ = 515.2 |
| 10-140 | 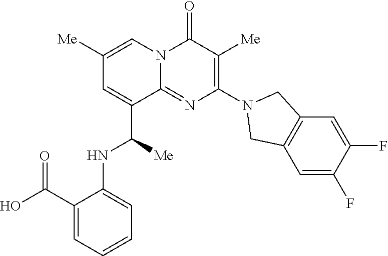 [SMILES: Cc1cc([C@@H](C)Nc2ccccc2C(=O)O)c2nc(N3Cc4cc(F)c(F)cc4C3)c(C)c(=O)n2c1] (R)-2-((1-(2-(5,6-difluoroisoindolin-2-yl)-3,7- dimethyl-4-oxo-4H-pyrido[1,2-a]pyrimidin-9- yl)ethyl)amino)benzoic acid | LCMS [M + 1]⁺ = 491.2 |
| 10-141 | 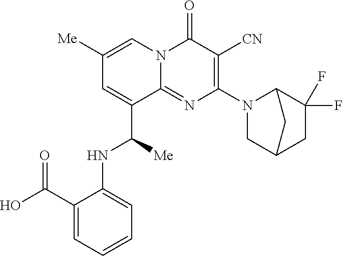 [SMILES: Cc1cc([C@@H](C)Nc2ccccc2C(=O)O)c2nc(N3CC4CC3C(F)(F)C4)c(C#N)c(=O)n2c1] 2-(((1R)-1-(2-(6,6-difluoro-2- azabicyclo[2.2.1]heptan-2-yl)-3,7-dimethyl-4- oxo-4H-pyrido[1,2-a]pyrimidin-9- yl)ethyl)amino)benzoic acid | LCMS [M + 1]⁺ = 469.2 |
| 10-142 | 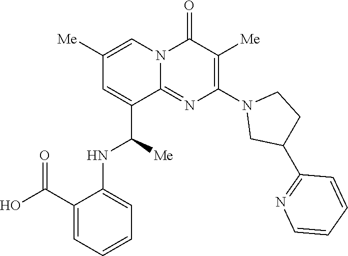 [SMILES: Cc1cc([C@@H](C)Nc2ccccc2C(=O)O)c2nc(N3CCC(c4ccccn4)C3)c(C)c(=O)n2c1] 2-(((1R)-1-(3,7-dimethyl-4-oxo-2-(3-(pyridin-2- yl)pyrrolidin-1-yl)-4H-pyrido[1,2-a]pyrimidin-9- yl)ethyl)amino)benzoic acid | LCMS [M + 1]⁺ = 484.2 |
| 10-143 | 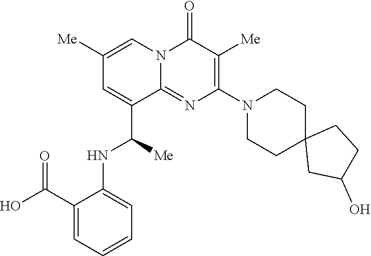 [SMILES: Cc1cc([C@@H](C)Nc2ccccc2C(=O)O)c2nc(N3CCC4(CCC(O)C4)CC3)c(C)c(=O)n2c1] 2-(((1R)-1-(2-(2-hydroxy-8-azaspiro[4.5]decan-8- yl)-3,7-dimethyl-4-oxo-4H-pyrido[1,2-a]pyrimidin- 9-yl)ethyl)amino)benzoic acid | LCMS [M + 1]⁺ = 491.1 |
| 10-144 | 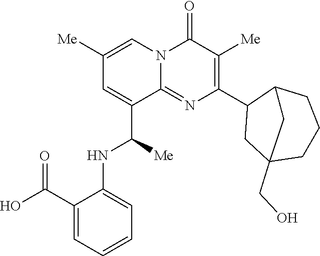 [SMILES: Cc1cc([C@@H](C)Nc2ccccc2C(=O)O)c2nc(C3CC4(CO)CCCC3C4)c(C)c(=O)n2c1] 2-(((1R)-1-(2-(1-(hydroxymethyl)-6- azabicyclo[3.2.1]octan-6-yl)-3,7-dimethyl-4-oxo- 4H-pyrido[1,2-a]pyrimidin-9- yl)ethyl)amino)benzoic acid | LCMS [M + 1]⁺ = 477.1 |
| 10-145 | 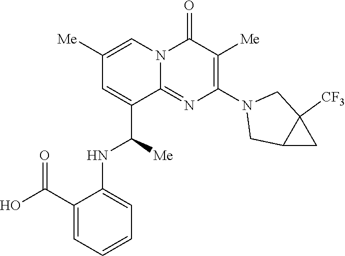 [SMILES: Cc1cc([C@@H](C)Nc2ccccc2C(=O)O)c2nc(N3CC4CC4(C(F)(F)F)C3)c(C)c(=O)n2c1] 2-(((1R)-1-(3,7-dimethyl-4-oxo-2-(1- (trifluoromethyl)-3-azabicyclo[3.1.0]hexan-3-yl)- 4H-pyrido[1,2-a]pyrimidin-9- yl)ethyl)amino)benzoic acid | LCMS [M + 1]⁺ = 487.2 |
| 10-146 | 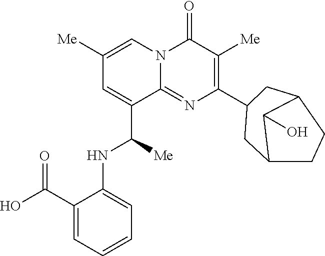 [SMILES: Cc1cc([C@@H](C)Nc2ccccc2C(=O)O)c2nc(C3CC4CCC(C3)C4O)c(C)c(=O)n2c1] 2-(((1R)-1-(2-(8-hydroxy-3- azabicyclo[3.2.1]octan-3-yl)-3,7-dimethyl-4-oxo- 4H-pyrido[1,2-a]pyrimidin-9- yl)ethyl)amino)benzoic acid | LCMS [M + 1]⁺ = 463.0 |
| 10-147 | 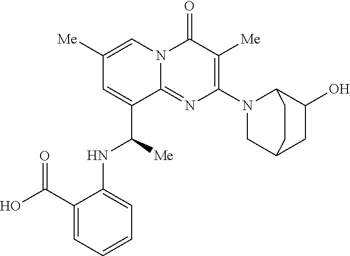 [SMILES: Cc1cc([C@@H](C)Nc2ccccc2C(=O)O)c2nc(N3CC4CCC3C(O)C4)c(C)c(=O)n2c1] 2-(((1R)-1-(2-(6-hydroxy-2- azabicyclo[2.2.2]octan-2-yl)-3,7-dimethyl-4-oxo- 4H-pyrido[1,2-a]pyrimidin-9- yl)ethyl)amino)benzoic acid | LCMS [M + 1]⁺ = 463.2 |
| 10-148 | 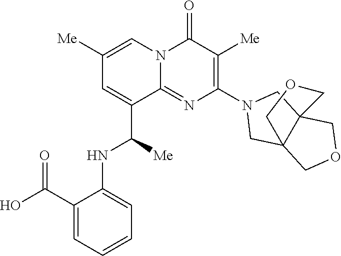 [SMILES: Cc1cc([C@@H](C)Nc2ccccc2C(=O)O)c2nc(N3CC45COCC4(COC5)C3)c(C)c(=O)n2c1] 2-(((R)-1-(2-((3aS,6aS)-1H,3H,4H-3a,6a- (methanooxymethano)furo[3,4-c]pyrrol-5(6H)-yl)- 3,7-dimethyl-4-oxo-4H-pyrido[1,2-a]pyrimidin-9- yl)ethyl)amino)benzoic acid | LCMS [M + 1]⁺ = 491.1 |

### Examples 11-1 to 11-447

The following compounds are prepared essentially according to the procedures set forth in the above schemes and examples.

| Example | Structure | Name |
|---|---|---|
| 11-1 | 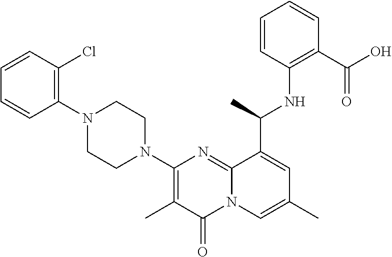 [SMILES: Cc1cc([C@@H](C)Nc2ccccc2C(=O)O)c2nc(N3CCN(c4ccccc4Cl)CC3)c(C)c(=O)n2c1] | (R)-2-((1-(2-(4-(2- chlorophenyl)piperazin-1-yl)-3,7- dimethyl-4-oxo-4H-pyrido[1,2-a]pyrimidin-9- yl)ethyl)amino)benzoic acid |
| 11-2 | 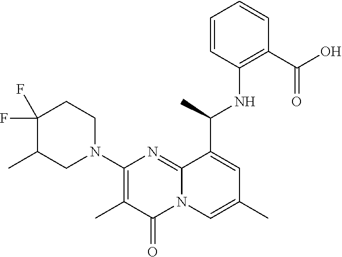 [SMILES: Cc1cc([C@@H](C)Nc2ccccc2C(=O)O)c2nc(N3CCC(F)(F)C(C)C3)c(C)c(=O)n2c1] | 2-(((1R)-1-(2-(4,4-difluoro-3- methylpiperidin-1-yl)-3,7- dimethyl-4-oxo-4H-pyrido[1,2-a]pyrimidin-9- yl)ethyl)amino)benzoic acid |
| 11-3 | 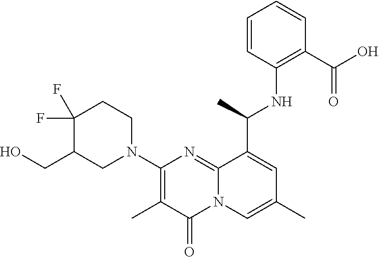 [SMILES: Cc1cc([C@@H](C)Nc2ccccc2C(=O)O)c2nc(N3CCC(F)(F)C(CO)C3)c(C)c(=O)n2c1] | 2-(((1R)-1-(2-(8,8-difluoro-3- (hydroxymethyl)piperidin-1-yl)- 3,7-dimethyl-4-oxo-4H- pyrido[1,2-a]pyrimidin-9- yl)ethyl)amino)benzoic acid |
| 11-4 | 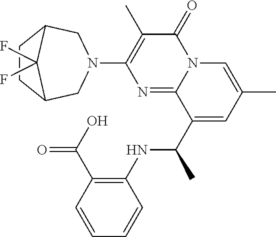 [SMILES: Cc1cc([C@@H](C)Nc2ccccc2C(=O)O)c2nc(N3CC4CCC(C3)C4(F)F)c(C)c(=O)n2c1] | 2-(((1R)-1-(3,7-dimethyl-4-oxo-2- azabicyclo[3.2.1]octan-3-yl)-3,7- dimethyl-4-oxo-4H-pyrido[1,2-a]pyrimidin-9- yl)ethyl)amino)benzoic acid |
| 11-5 | 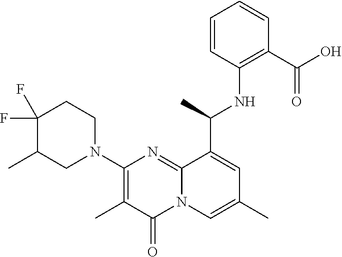 [SMILES: Cc1cc([C@@H](C)Nc2ccccc2C(=O)O)c2nc(N3CCC(F)(F)C(C)C3)c(C)c(=O)n2c1] | 2-(((1R)-1-(3,7-dimethyl-4-oxo-2- (3,4,4-trifluoropiperidin-1-yl)-4H- pyrido[1,2-a]pyrimidin-9- yl)ethyl)amino)benzoic acid |
| 11-6 | 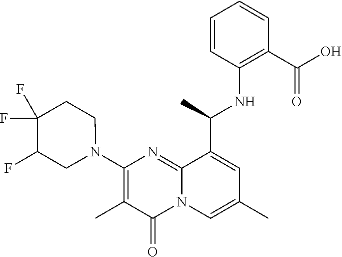 [SMILES: Cc1cc([C@@H](C)Nc2ccccc2C(=O)O)c2nc(N3CCC(F)(F)C(F)C3)c(C)c(=O)n2c1] | 2-(((1R)-1-(2-(9,9-difluoro-3-oxa- 7-azabicyclo[3.3.1]nonan-7-yl)- 3,7-dimethyl-4-oxo-4H-pyrido[1,2-a]pyrimidin-9- yl)ethyl)amino)benzoic acid |
| 11-7 | 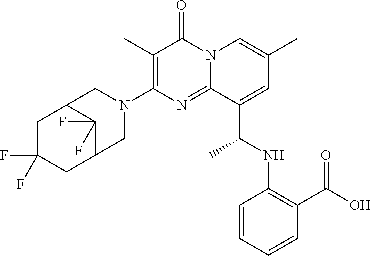 [SMILES: Cc1cc([C@@H](C)Nc2ccccc2C(=O)O)c2nc(N3CC4CC(F)(F)CC(C3)C4(F)F)c(C)c(=O)n2c1] | 2-(((1R)-1-(3,7-dimethyl-4-oxo-2- (7,7,9,9-tetrafluoro-3- azabicyclo[3.3.1]nonan-3-yl)-4H- pyrido[1,2-a]pyrimidin-9- yl)ethyl)amino)benzoic acid |
| 11-8 | 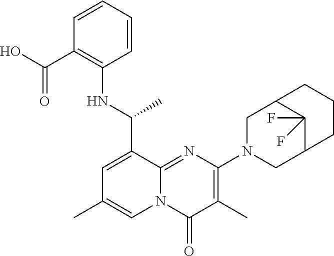 [SMILES: Cc1cc([C@@H](C)Nc2ccccc2C(=O)O)c2nc(N3CC4CCCC(C3)C4(F)F)c(C)c(=O)n2c1] | 2-(((1R)-1-(2-(9,9-difluoro-3- azabicyclo[3.3.1]nonan-3-yl)-3,7- dimethyl-4-oxo-4H- pyrido[1,2-a]pyrimidin-9- yl)ethyl)amino)benzoic acid |
| 11-9 | 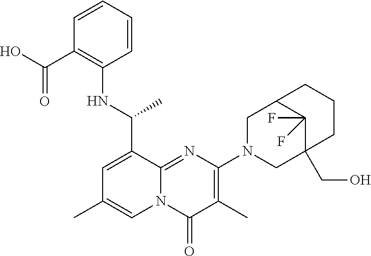 [SMILES: Cc1cc([C@@H](C)Nc2ccccc2C(=O)O)c2nc(N3CC4CCCC(CO)(C3)C4(F)F)c(C)c(=O)n2c1] | 2-(((1R)-1-(2-(9,9-difluoro-1- (hydroxymethyl)-3- azabicyclo[3.3.1]nonan-3-yl)-3,7- dimethyl-4-oxo-4H- pyrido[1,2-a]pyrimidin-9- yl)ethyl)amino)benzoic acid |
| 11-10 | 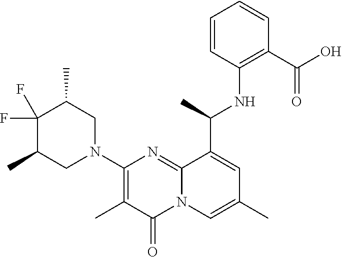 [SMILES: Cc1cc([C@@H](C)Nc2ccccc2C(=O)O)c2nc(N3C[C@@H](C)C(F)(F)[C@H](C)C3)c(C)c(=O)n2c1] | 2-(((R)-1-(2-((3R,5R)-4,4- difluoro-3,5-dimethylpiperidin-1- yl)-3,7-dimethyl-4-oxo-4H- pyrido[1,2-a]pyrimidin-9- yl)ethyl)amino)benzoic acid And 2-(((1R)-1-(2-((3S,5S)-4,4- difluoro-3,5-dimethylpiperidin-1- yl)-3,7-dimethyl-4-oxo-4H- pyrido[1,2-a]pyrimidin-9- yl)ethyl)amino)benzoic acid |
| 11-11 | 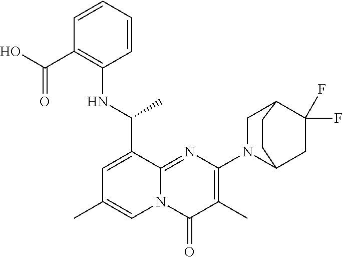 [SMILES: Cc1cc([C@@H](C)Nc2ccccc2C(=O)O)c2nc(N3CC4CCC3CC4(F)F)c(C)c(=O)n2c1] | 2-(((1R)-1-(2-(5,5-difluoro-2- azabicyclo[2.2.2]octan-2-yl)-3,7- dimethyl-4-oxo-4H- pyrido[1,2-a]pyrimidin-9- yl)ethyl)amino)benzoic acid |
| 11-12 | 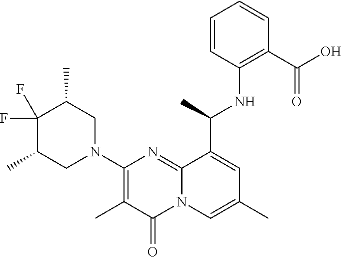 [SMILES: Cc1cc([C@@H](C)Nc2ccccc2C(=O)O)c2nc(N3C[C@@H](C)C(F)(F)[C@@H](C)C3)c(C)c(=O)n2c1] | 2-(((R)-1-(2-((3R,5S)-4,4- difluoro-3,5-dimethylpiperidin-1- yl)-3,7-dimethyl-4-oxo-4H- pyrido[1,2-a]pyrimidin-9- yl)ethyl)amino)benzoic acid And 2-(((R)-1-(2-((3S,5R)-4,4- difluoro-3,5-dimethylpiperidin-1- yl)-3,7-dimethyl-4-oxo-4H- pyrido[1,2-a]pyrimidin-9- yl)ethyl)amino)benzoic acid |
| 11-13 | 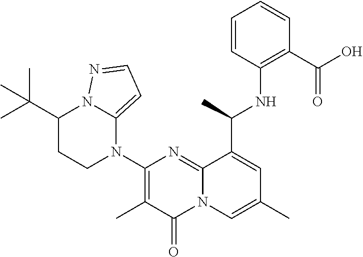 [SMILES: Cc1cc([C@@H](C)Nc2ccccc2C(=O)O)c2nc(N3CCC(C(C)(C)C)n4nccc43)c(C)c(=O)n2c1] | 2-(((1R)-1-(2-(7-(tert-butyl)-6,7- dihydropyrazolo[1,5-a]pyrimidin- 4(5H)-yl)-3,7-dimethyl-4-oxo-4H- pyrido[1,2-a]pyrimidin-9- yl)ethyl)amino)benzoic acid |
| 11-14 | 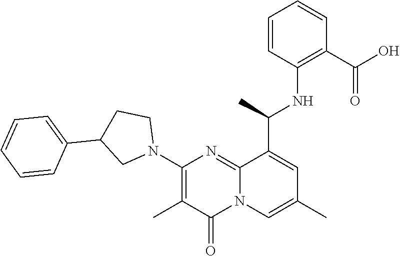 [SMILES: Cc1cc([C@@H](C)Nc2ccccc2C(=O)O)c2nc(N3CCC(c4ccccc4)C3)c(C)c(=O)n2c1] | 2-(((1R)-1-(3,7-dimethyl-4-oxo-2- (3-phenylpyrrolidin-1-yl)-4H- pyrido[1,2-a]pyrimidin-9- yl)ethyl)amino)benzoic acid |
| 11-15 | 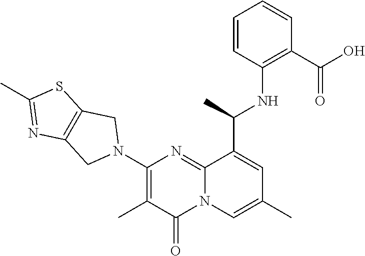 [SMILES: Cc1cc([C@@H](C)Nc2ccccc2C(=O)O)c2nc(N3Cc4nc(C)sc4C3)c(C)c(=O)n2c1] | (R)-2-((1-(3,7-dimethyl-2-(2- methyl-4,6-dihydro-5H- pyrrolo[3,4-d]thiazol-5-yl)-4-oxo- 4H-pyrido[1,2-a]pyrimidin-9- yl)ethyl)amino)benzoic acid |
| 11-16 | 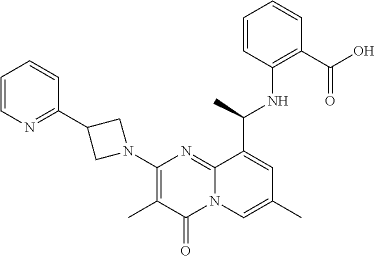 [SMILES: Cc1cc([C@@H](C)Nc2ccccc2C(=O)O)c2nc(N3CC(c4ccccn4)C3)c(C)c(=O)n2c1] | (R)-2-((1-(3,7-dimethyl-4-oxo-2- (3-(pyridin-2-yl)azetidin-1-yl)-4H- pyrido[1,2-a]pyrimidin-9- yl)ethyl)amino)benzoic acid |
| 11-17 | 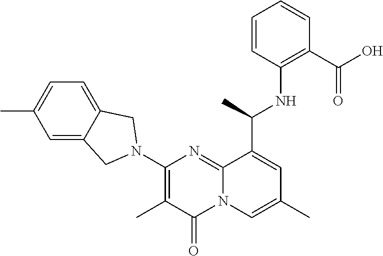 [SMILES: Cc1ccc2c(c1)CN(c1nc3c([C@@H](C)Nc4ccccc4C(=O)O)cc(C)cn3c(=O)c1C)C2] | (R)-2-((1-(3,7-dimethyl-2-(5- methylisoindolin-2-yl)-4-oxo- 4H-pyrido[1,2-a]pyrimidin-9- yl)ethyl)amino)benzoic acid |
| 11-18 | 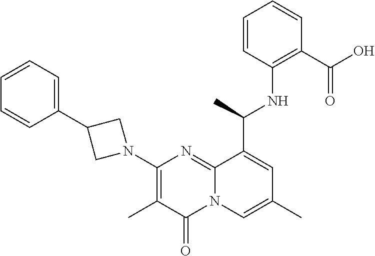 [SMILES: Cc1cc([C@@H](C)Nc2ccccc2C(=O)O)c2nc(N3CC(c4ccccc4)C3)c(C)c(=O)n2c1] | (R)-2-((1-(3,7-dimethyl-4-oxo-2- (3-phenylazetidin-1-yl)- 4H-pyrido[1,2-a]pyrimidin-9- yl)ethyl)amino)benzoic acid |
| 11-19 | 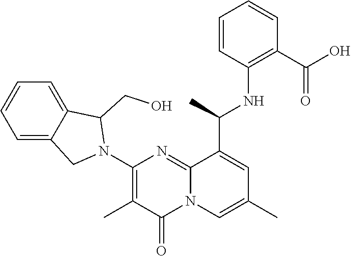 [SMILES: Cc1cc([C@@H](C)Nc2ccccc2C(=O)O)c2nc(N3Cc4ccccc4C3CO)c(C)c(=O)n2c1] | 2-(((1R)-1-(2-(1- (hydroxymethyl)isoindolin-2-yl)- 3,7-dimethyl-4-oxo- 4H-pyrido[1,2-a]pyrimidin-9- yl)ethyl)amino)benzoic acid |
| 11-20 | 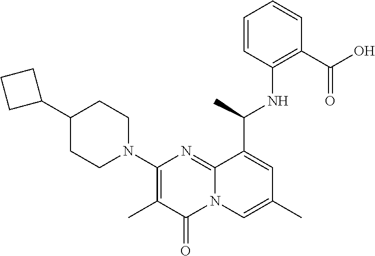 [SMILES: Cc1cc([C@@H](C)Nc2ccccc2C(=O)O)c2nc(N3CCC(C4CCC4)CC3)c(C)c(=O)n2c1] | (R)-2-((1-(2-(4- cyclobutylpiperidin-1-yl)-3,7-dimethyl-4-oxo- 4H-pyrido[1,2-a]pyrimidin-9- yl)ethyl)amino)benzoic acid |
| 11-21 | 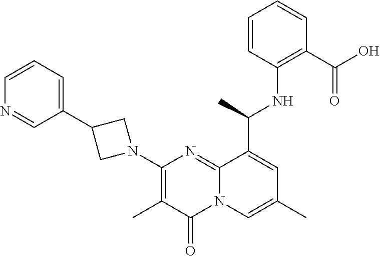 [SMILES: Cc1cc([C@@H](C)Nc2ccccc2C(=O)O)c2nc(N3CC(c4cccnc4)C3)c(C)c(=O)n2c1] | (R)-2-((1-(3,7-dimethyl-4-oxo-2- (3-(pyridin-3-yl)azetidin-1-yl)- 4H-pyrido[1,2-a]pyrimidin-9- yl)ethyl)amino)benzoic acid |
| 11-22 | 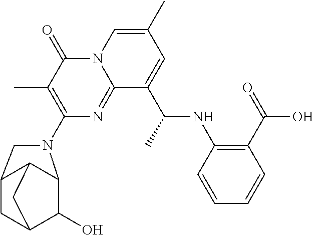 [SMILES: Cc1cc([C@@H](C)Nc2ccccc2C(=O)O)c2nc(N3CC4CC5CC4C3C5O)c(C)c(=O)n2c1] | 2-(((1R)-1-(2-(6-hydroxyhexahydro-3,5- methanocyclopenta[b]pyrrol- 1(2H)-yl)-3,7-dimethyl-4-oxo- 4H-pyrido[1,2-a]pyrimidin-9- yl)ethyl)amino)benzoic acid |
| 11-23 | 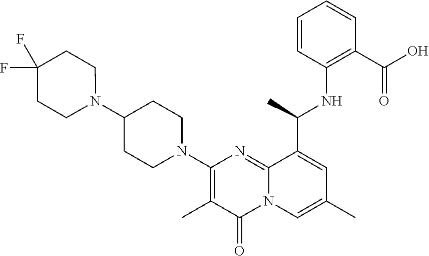 [SMILES: Cc1cc([C@@H](C)Nc2ccccc2C(=O)O)c2nc(N3CCC(N4CCC(F)(F)CC4)CC3)c(C)c(=O)n2c1] | (R)-2-((1-(2-(4,4-difluoro-[1,4′- bipiperidin]-1′-yl)-3,7-dimethyl-4- oxo-4H-pyrido[1,2-a]pyrimidin-9- yl)ethyl)amino)benzoic acid |
| 11-24 | 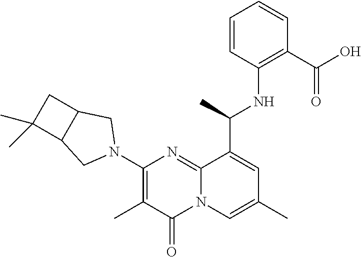 [SMILES: Cc1cc([C@@H](C)Nc2ccccc2C(=O)O)c2nc(N3CC4CC(C)(C)C4C3)c(C)c(=O)n2c1] | 2-(((1R)-1-(2-(6,6-dimethyl-3- azabicyclo[3.2.0]heptan-3-yl)- 3,7-dimethyl-4-oxo- 4H-pyrido[1,2-a]pyrimidin-9- yl)ethyl)amino)benzoic acid |
| 11-25 | 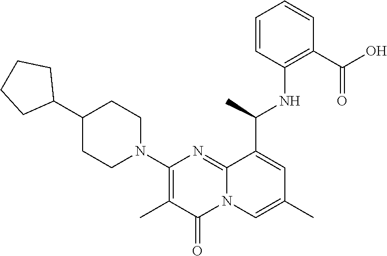 [SMILES: Cc1cc([C@@H](C)Nc2ccccc2C(=O)O)c2nc(N3CCC(C4CCCC4)CC3)c(C)c(=O)n2c1] | (R)-2-((1-(2-(4- cyclopentylpiperidin-1-yl)-3,7-dimethyl-4-oxo- 4H-pyrido[1,2-a]pyrimidin-9- yl)ethyl)amino)benzoic acid |
| 11-26 | 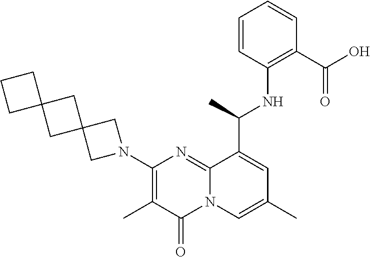 [SMILES: Cc1cc([C@@H](C)Nc2ccccc2C(=O)O)c2nc(N3CC4(C3)CC3(CCC3)C4)c(C)c(=O)n2c1] | (R)-2-((1-(2-(2-azadispiro[3.1.3⁶.1⁴]decan-2-yl)- 3,7-dimethyl-4-oxo-4H-pyrido[1,2-a]pyrimidin-9- yl)ethyl)amino)benzoic acid |
| 11-27 | 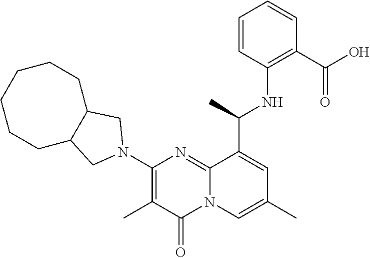 [SMILES: Cc1cc([C@@H](C)Nc2ccccc2C(=O)O)c2nc(N3CC4CCCCCCC4C3)c(C)c(=O)n2c1] | 2-(((1R)-1-(2-(decahydro-2H- cycloocta[c]pyrrol-2-yl)-3,7-dimethyl-4-oxo- 4H-pyrido[1,2-a]pyrimidin-9- yl)ethyl)amino)benzoic acid |
| 11-28 | 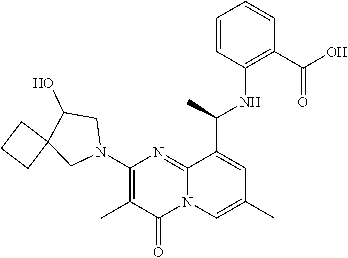 [SMILES: Cc1cc([C@@H](C)Nc2ccccc2C(=O)O)c2nc(N3CC(O)C4(CCC4)C3)c(C)c(=O)n2c1] | 2-(((1R)-1-(2-(8-hydroxy-6- azaspiro[3.4]octan-6-yl)-3,7-dimethyl-4-oxo- 4H-pyrido[1,2-a]pyrimidin-9- yl)ethyl)amino)benzoic acid |
| 11-29 | 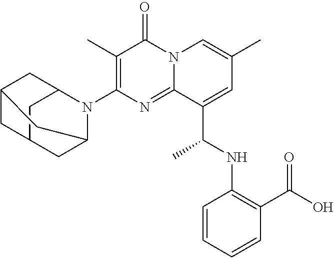 [SMILES: Cc1cc([C@@H](C)Nc2ccccc2C(=O)O)c2nc(N3C4CC5CC(C4)CC3C5)c(C)c(=O)n2c1] | (R)-2-((1-(2-(2-azaadamantan-2- yl)-3,7-dimethyl-4-oxo- 4H-pyrido[1,2-a]pyrimidin-9- yl)ethyl)amino)benzoic acid |
| 11-30 | 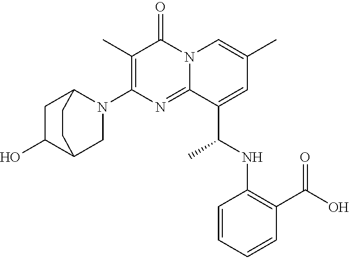 [SMILES: Cc1cc([C@@H](C)Nc2ccccc2C(=O)O)c2nc(N3CC4CCC3CC4O)c(C)c(=O)n2c1] | 2-(((1R)-1-(2-(5-hydroxy-2- azabicyclo[2.2.2]octan-2-yl)-3,7-dimethyl-4-oxo- 4H-pyrido[1,2-a]pyrimidin-9- yl)ethyl)amino)benzoic acid |
| 11-31 | 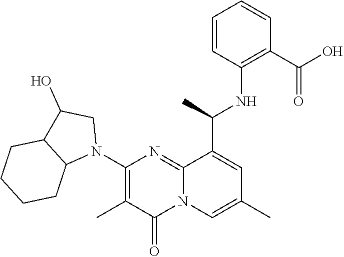 [SMILES: Cc1cc([C@@H](C)Nc2ccccc2C(=O)O)c2nc(N3CC(O)C4CCCCC43)c(C)c(=O)n2c1] | 2-(((1R)-1-(2-(3- hydroxyoctahydro-1H-indol-1-yl)- 3,7-dimethyl-4-oxo- 4H-pyrido[1,2-a]pyrimidin-9- yl)ethyl)amino)benzoic acid |
| 11-32 | 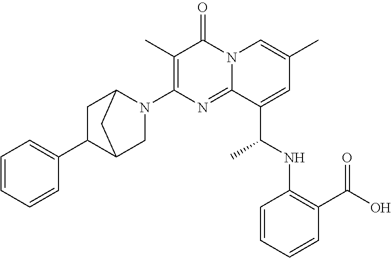 [SMILES: Cc1cc([C@@H](C)Nc2ccccc2C(=O)O)c2nc(N3CC4CC3CC4c3ccccc3)c(C)c(=O)n2c1] | 2-(((1R)-1-(3,7-dimethyl-4-oxo-2- (5-phenyl-2-azabicyclo[2.2.1]heptan-2-yl)- 4H-pyrido[1,2-a]pyrimidin-9- yl)ethyl)amino)benzoic acid |
| 11-33 | 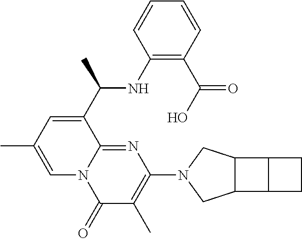 [SMILES: Cc1cc([C@@H](C)Nc2ccccc2C(=O)O)c2nc(N3CC4C5CCC5C4C3)c(C)c(=O)n2c1] | 2-(((1R)-1-(3,7-dimethyl-4-oxo-2- (8-azatricyclo[4.3.0.0^{2,5}]nonan-8- yl)-4H-pyrido[1,2-a]pyrimidin-9- yl)ethyl)amino)benzoic acid |
| 11-34 | 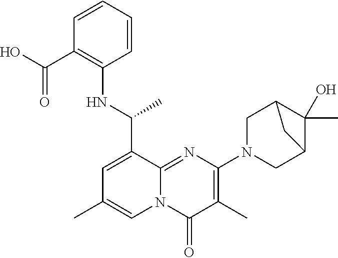 [SMILES: Cc1cc([C@@H](C)Nc2ccccc2C(=O)O)c2nc(N3CC4CC(C3)C4(C)O)c(C)c(=O)n2c1] | 2-(((1R)-1-(2-(6-hydroxy-6-methyl-3- azabicyclo[3.1.1]heptan-3-yl)- 3,7-dimethyl-4-oxo- 4H-pyrido[1,2-a]pyrimidin-9- yl)ethyl)amino)benzoic acid |
| 11-35 | 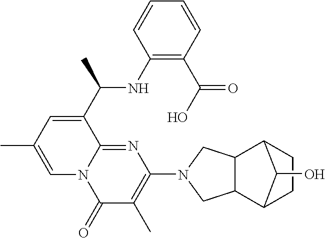 [SMILES: Cc1cc([C@@H](C)Nc2ccccc2C(=O)O)c2nc(N3CC4C5CCC(C5O)C4C3)c(C)c(=O)n2c1] | 2-(((1R)-1-(2-(8- hydroxyoctahydro-2H-4,7- methanoisoindol-2-yl)-3,7-dimethyl-4-oxo- 4H-pyrido[1,2-a]pyrimidin-9- yl)ethyl)amino)benzoic acid |
| 11-36 | 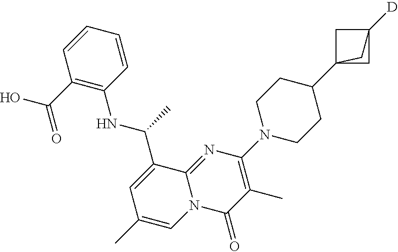 [SMILES: [2H]C12CC(C3CCN(c4nc5c([C@@H](C)Nc6ccccc6C(=O)O)cc(C)cn5c(=O)c4C)CC3)(C1)C2] | (R)-2-((1-(2-(4- (bicyclo[1.1.1]pentan-1-yl-3- d)piperidin-1-yl)-3,7-dimethyl-4- oxo-4H-pyrido[1,2-a]pyrimidin-9- yl)ethyl)amino)benzoic acid |
| 11-37 | 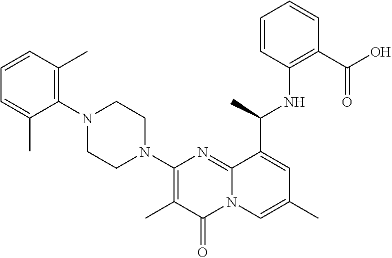 [SMILES: Cc1cc([C@@H](C)Nc2ccccc2C(=O)O)c2nc(N3CCN(c4c(C)cccc4C)CC3)c(C)c(=O)n2c1] | (R)-2-((1-(2-(4-(2,6- dimethylphenyl)piperazin-1-yl)- 3,7-dimethyl-4-oxo- 4H-pyrido[1,2-a]pyrimidin-9- yl)ethyl)amino)benzoic acid |
| 11-38 | 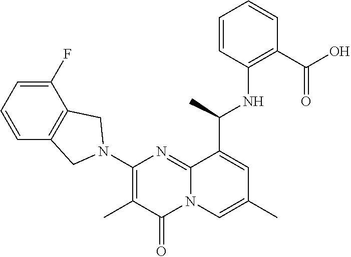 [SMILES: Cc1cc([C@@H](C)Nc2ccccc2C(=O)O)c2nc(N3Cc4cccc(F)c4C3)c(C)c(=O)n2c1] | (R)-2-((1-(2-(4-fluoroisoindolin-2- yl)-3,7-dimethyl-4-oxo- 4H-pyrido[1,2-a]pyrimidin-9- yl)ethyl)amino)benzoic acid |
| 11-39 | 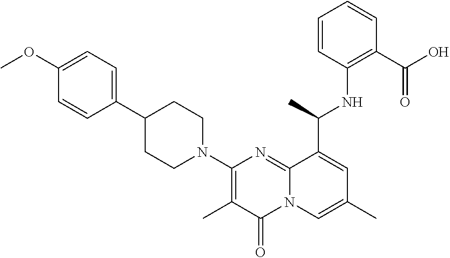 [SMILES: COc1ccc(C2CCN(c3nc4c([C@@H](C)Nc5ccccc5C(=O)O)cc(C)cn4c(=O)c3C)CC2)cc1] | (R)-2-((1-(2-(4-(4- methoxyphenyl)piperidin-1-yl)- 3,7-dimethyl-4-oxo- 4H-pyrido[1,2-a]pyrimidin-9- yl)ethyl)amino)benzoic acid |
| 11-40 | 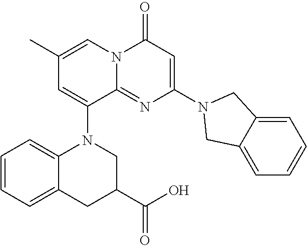 [SMILES: Cc1cc(N2CC(C(=O)O)Cc3ccccc32)c2nc(N3Cc4ccccc4C3)cc(=O)n2c1] | 1-(2-(isoindolin-2-yl)-7-methyl-4- oxo-4H-pyrido[1,2-a]pyrimidin-9- yl)-1,2,3,4-tetrahydroquinoline-3- carboxylic acid |
| 11-41 | 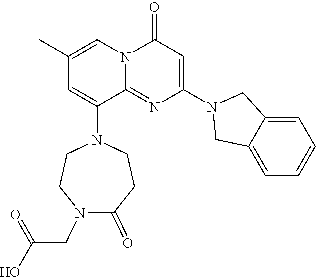 [SMILES: Cc1cc(N2CCC(=O)N(CC(=O)O)CC2)c2nc(N3Cc4ccccc4C3)cc(=O)n2c1] | 2-(4-(2-(isoindolin-2-yl)-7-methyl- 4-oxo-4H-pyrido[1,2-a]pyrimidin- 9-yl)-7-oxo-1,4-diazepan-1- yl)acetic acid |
| 11-42 | 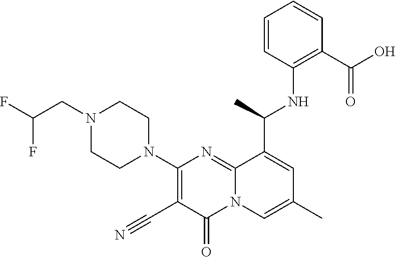 [SMILES: Cc1cc([C@@H](C)Nc2ccccc2C(=O)O)c2nc(N3CCN(CC(F)F)CC3)c(C#N)c(=O)n2c1] | (R)-2-((1-(3-cyano-2-(4-(2,2- difluoroethyl)piperazin-1-yl)-7- methyl-4-oxo-4H-pyrido[1,2-a] pyrimidin-9- yl)ethyl)amino)benzoic acid |
| 11-43 | 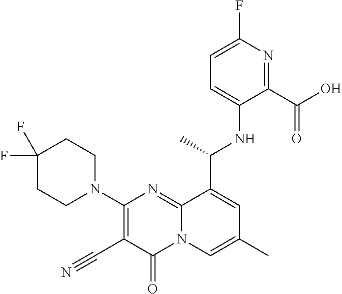 [SMILES: Cc1cc([C@H](C)Nc2ccc(F)nc2C(=O)O)c2nc(N3CCC(F)(F)CC3)c(C#N)c(=O)n2c1] | (S)-3-((1-(3-cyano-2-(4,4- difluoropiperidin-1-yl)-7-methyl- 4-oxo-4H-pyrido[1,2-a]pyrimidin- 9-yl)ethyl)amino)-6- fluoropicolinic acid |
| 11-44 | 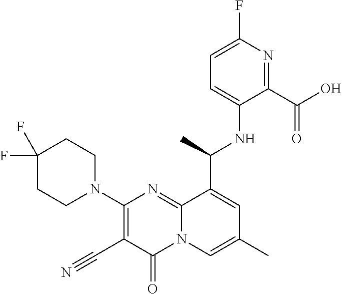 [SMILES: Cc1cc([C@@H](C)Nc2ccc(F)nc2C(=O)O)c2nc(N3CCC(F)(F)CC3)c(C#N)c(=O)n2c1] | (R)-3-((1-(3-cyano-2-(4,4- difluoropiperidin-1-yl)-7-methyl- 4-oxo-4H-pyrido[1,2-a]pyrimidin-9- yl)ethyl)amino)-6- fluoropicolinic acid |
| 11-45 | 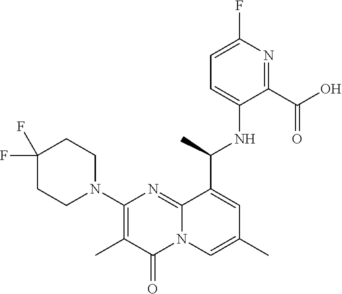 [SMILES: Cc1cc([C@@H](C)Nc2ccc(F)nc2C(=O)O)c2nc(N3CCC(F)(F)CC3)c(C)c(=O)n2c1] | (R)-3-((1-(2-(4,4- difluoropiperidin-1-yl)-3,7- dimethyl-4-oxo-4H-pyrido[1,2- a]pyrimidin-9-yl)ethyl)amino)-6- fluoropicolinic acid |
| 11-46 | 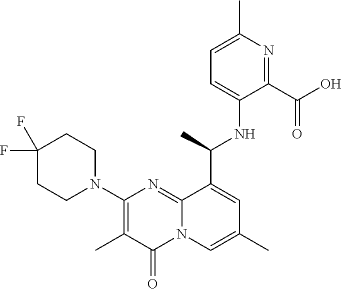 [SMILES: Cc1cc([C@@H](C)Nc2ccc(C)nc2C(=O)O)c2nc(N3CCC(F)(F)CC3)c(C)c(=O)n2c1] | (R)-3-((1-(2-(4,4- difluoropiperidin-1-yl)-3,7- dimethyl-4-oxo-4H-pyrido[1,2- a]pyrimidin-9-yl)ethyl)amino)-6- methylpicolinic acid |
| 11-47 | 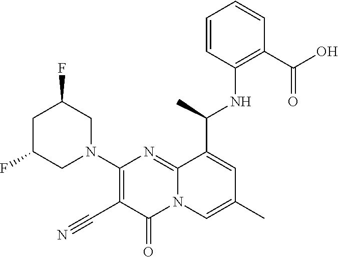 [SMILES: Cc1cc([C@@H](C)Nc2ccccc2C(=O)O)c2nc(N3C[C@H](F)C[C@@H](F)C3)c(C#N)c(=O)n2c1] | 2-(((R)-1-(3-cyano-2-((3R,5R)- 3,5-difluoropiperidin-1-yl)-7- methyl-4-oxo- 4H-pyrido[1,2-a]pyrimidin-9- yl)ethyl)amino)benzoic acid |
| 11-48 | 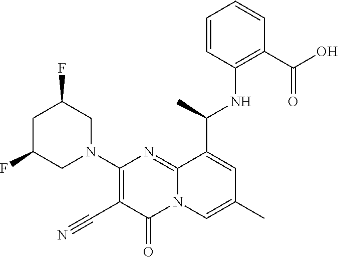 [SMILES: Cc1cc([C@@H](C)Nc2ccccc2C(=O)O)c2nc(N3C[C@H](F)C[C@H](F)C3)c(C#N)c(=O)n2c1] | 2-(((R)-1-(3-cyano-2-((3R,5S)- 3,5-difluoropiperidin-1-yl)-7- methyl-4-oxo- 4H-pyrido[1,2-a]pyrimidin-9- yl)ethyl)amino)benzoic acid |
| 11-49 | 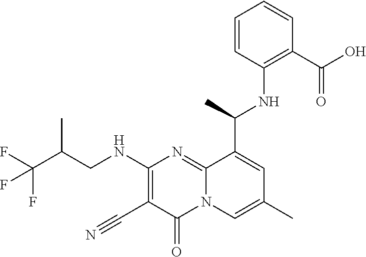 [SMILES: Cc1cc([C@@H](C)Nc2ccccc2C(=O)O)c2nc(NCC(C)C(F)(F)F)c(C#N)c(=O)n2c1] | 2-(((1R)-1-(3-cyano-7-methyl-4- oxo-2-((3,3,3-trifluoro-2- methylpropyl)amino)- 4H-pyrido[1,2-a]pyrimidin-9- yl)ethyl)amino)benzoic acid |
| 11-50 | 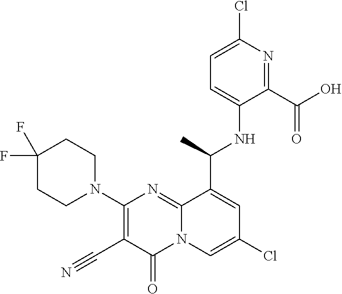 [SMILES: C[C@@H](Nc1ccc(Cl)nc1C(=O)O)c1cc(Cl)cn2c(=O)c(C#N)c(N3CCC(F)(F)CC3)nc12] | (R)-6-chloro-3-((1-(7-chloro-3- cyano-2-(4,4-difluoropiperidin-1- yl)-4-oxo-4H-pyrido [1,2-a]pyrimidin-9- yl)ethyl)amino)picolinic acid |
| 11-51 | 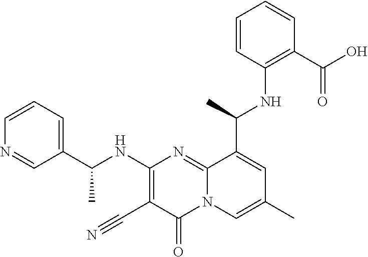 [SMILES: Cc1cc([C@@H](C)Nc2ccccc2C(=O)O)c2nc(N[C@H](C)c3cccnc3)c(C#N)c(=O)n2c1] | 2-(((R)-1-(3-cyano-7-methyl-4- oxo-2-(((R)-1-(pyridin-3- yl)ethyl)amino)- 4H-pyrido[1,2-a]pyrimidin-9- yl)ethyl)amino)benzoic acid |
| 11-52 | 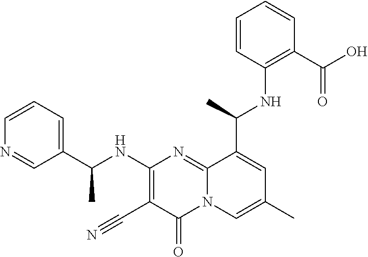 [SMILES: Cc1cc([C@@H](C)Nc2ccccc2C(=O)O)c2nc(N[C@@H](C)c3cccnc3)c(C#N)c(=O)n2c1] | 2-(((R)-1-(3-cyano-7-methyl-4- oxo-2-(((S)-1-(pyridin-3- yl)ethyl)amino)- 4H-pyrido[1,2-a]pyrimidin-9- yl)ethyl)amino)benzoic acid |
| 11-53 | 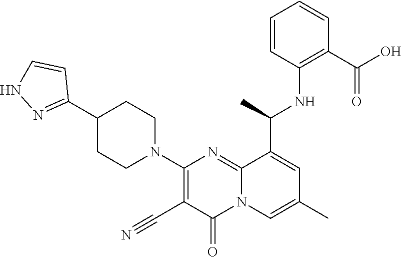 [SMILES: Cc1cc([C@@H](C)Nc2ccccc2C(=O)O)c2nc(N3CCC(c4cc[nH]n4)CC3)c(C#N)c(=O)n2c1] | (R)-2-((1-(2-(4-(1H-pyrazol-3- yl)piperidin-1-yl)-3-cyano-7- methyl-4-oxo- 4H-pyrido[1,2-a]pyrimidin-9- yl)ethyl)amino)benzoic acid |
| 11-54 | 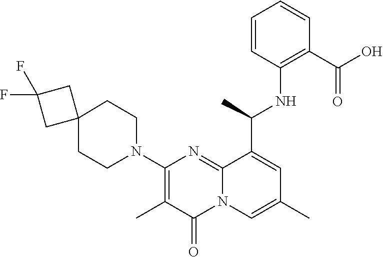 [SMILES: Cc1cc([C@@H](C)Nc2ccccc2C(=O)O)c2nc(N3CCC4(CC3)CC(F)(F)C4)c(C)c(=O)n2c1] | (R)-2-((1-(2-(2,2-difluoro-7- azaspiro[3.5]nonan-7-yl)-3,7- dimethyl-4-oxo-4H-pyrido[1,2- a]pyrimidin-9- yl)ethyl)amino)benzoic acid |
| 11-55 | 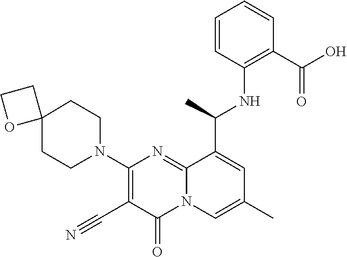 [SMILES: Cc1cc([C@@H](C)Nc2ccccc2C(=O)O)c2nc(N3CCC4(CCO4)CC3)c(C#N)c(=O)n2c1] | (R)-2-((1-(3-cyano-7-methyl-4- oxo-2-(1-oxa-7- azaspiro[3.5]nonan-7-yl)- 4H-pyrido[1,2-a]pyrimidin-9- yl)ethyl)amino)benzoic acid |
| 11-56 | 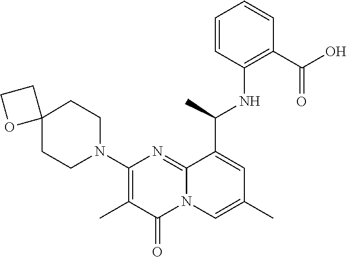 [SMILES: Cc1cc([C@@H](C)Nc2ccccc2C(=O)O)c2nc(N3CCC4(CCO4)CC3)c(C)c(=O)n2c1] | (R)-2-((1-(3,7-dimethyl-4-oxo-2- (1-oxa-7-azaspiro[3.5]nonan-7- yl)-4H-pyrido[1,2-a]pyrimidin-9- yl)ethyl)amino)benzoic acid |
| 11-57 | 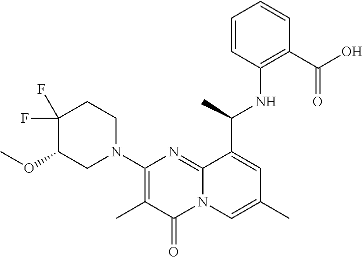 [SMILES: CO[C@H]1CN(c2nc3c([C@@H](C)Nc4ccccc4C(=O)O)cc(C)cn3c(=O)c2C)CCC1(F)F] | 2-(((R)-1-(2-((S)-4,4-difluoro-3- methoxypiperidin-1-yl)-3,7- dimethyl-4-oxo-4H-pyrido[1,2-a]pyrimidin-9- yl)ethyl)amino)benzoic acid |
| 11-58 | 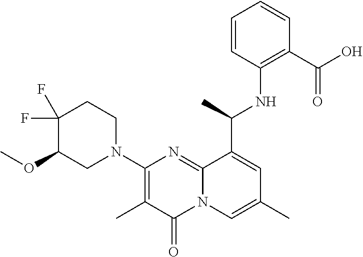 [SMILES: CO[C@@H]1CN(c2nc3c([C@@H](C)Nc4ccccc4C(=O)O)cc(C)cn3c(=O)c2C)CCC1(F)F] | 2-(((R)-1-(2-((R)-4,4-difluoro-3- methoxypiperidin-1-yl)-3,7- dimethyl-4-oxo- 4H-pyrido[1,2-a]pyrimidin-9- yl)ethyl)amino)benzoic acid |
| 11-59 | 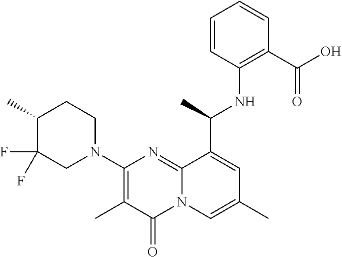 [SMILES: Cc1cc([C@@H](C)Nc2ccccc2C(=O)O)c2nc(N3CC[C@@H](C)C(F)(F)C3)c(C)c(=O)n2c1] | 2-(((R)-1-(2-((R)-3,3-difluoro-4- methylpiperidin-1-yl)-3,7- dimethyl-4-oxo- 4H-pyrido[1,2-a]pyrimidin-9- yl)ethyl)amino)benzoic acid |
| 11-60 | 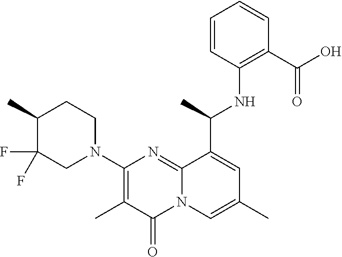 [SMILES: Cc1cc([C@@H](C)Nc2ccccc2C(=O)O)c2nc(N3CC[C@H](C)C(F)(F)C3)c(C)c(=O)n2c1] | 2-(((R)-1-(2-((S)-3,3-difluoro-4- methylpiperidin-1-yl)-3,7- dimethyl-4-oxo- 4H-pyrido[1,2-a]pyrimidin-9- yl)ethyl)amino)benzoic acid |
| 11-61 | 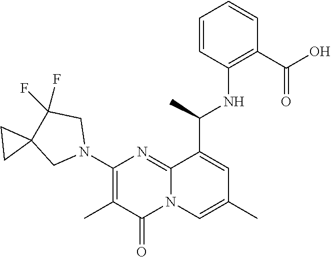 [SMILES: Cc1cc([C@@H](C)Nc2ccccc2C(=O)O)c2nc(N3CC(F)(F)C4(CC4)C3)c(C)c(=O)n2c1] | (R)-2-((1-(2-(7,7-difluoro-5- azaspiro[2.4]heptan-5-yl)-3,7- dimethyl-4-oxo- 4H-pyrido[1,2-a]pyrimidin-9- yl)ethyl)amino)benzoic acid |
| 11-62 | 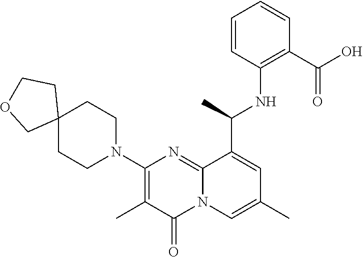 [SMILES: Cc1cc([C@@H](C)Nc2ccccc2C(=O)O)c2nc(N3CCC4(CCOC4)CC3)c(C)c(=O)n2c1] | (R)-2-((1-(3,7-dimethyl-4-oxo-2- (2-oxa-8-azaspiro[4.5]decan-8- yl)-4H-pyrido[1,2-a]pyrimidin-9- yl)ethyl)amino)benzoic acid |
| 11-63 | 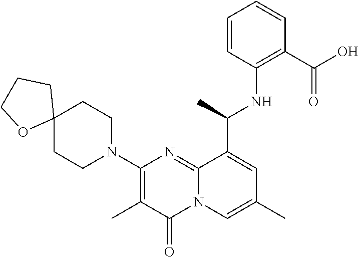 [SMILES: Cc1cc([C@@H](C)Nc2ccccc2C(=O)O)c2nc(N3CCC4(CCCO4)CC3)c(C)c(=O)n2c1] | (R)-2-((1-(3,7-dimethyl-4-oxo-2- (1-oxa-8-azaspiro[4.5]decan-8- yl)-4H-pyrido[1,2-a]pyrimidin-9- yl)ethyl)amino)benzoic acid |
| 11-64 | 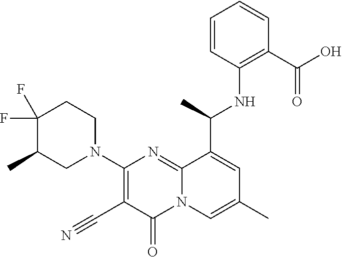 [SMILES: Cc1cc([C@@H](C)Nc2ccccc2C(=O)O)c2nc(N3CCC(F)(F)[C@H](C)C3)c(C#N)c(=O)n2c1] | 2-(((R)-1-(3-cyano-2-((R)-4,4- difluoro-3-methylpiperidin-1-yl)- 7-methyl-4-oxo- 4H-pyrido[1,2-a]pyrimidin-9- yl)ethyl)amino)benzoic acid |
| 11-65 | 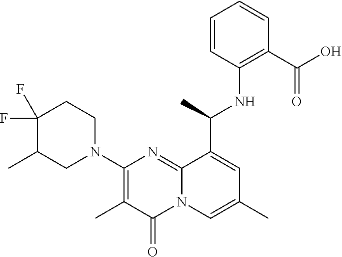 [SMILES: Cc1cc([C@@H](C)Nc2ccccc2C(=O)O)c2nc(N3CCC(F)(F)C(C)C3)c(C)c(=O)n2c1] | 2-(((R)-1-(2-(4,4-difluoro-3- methylpiperidin-1-yl)-3,7- dimethyl-4-oxo- 4H-pyrido[1,2-a]pyrimidin-9- yl)ethyl)amino)benzoic acid |
| 11-66 | 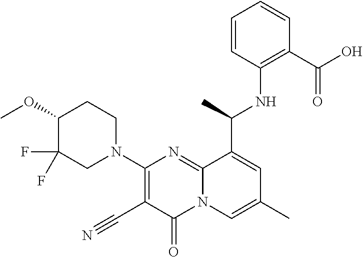 [SMILES: CO[C@@H]1CCN(c2nc3c([C@@H](C)Nc4ccccc4C(=O)O)cc(C)cn3c(=O)c2C#N)CC1(F)F] | 2-(((R)-1-(3-cyano-2-((R)-3,3- difluoro-4-methoxypiperidin-1-yl)- 7-methyl-4-oxo- 4H-pyrido[1,2-a]pyrimidin-9- yl)ethyl)amino)benzoic acid |
| 11-67 | 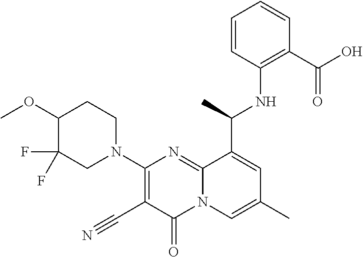 [SMILES: COC1CCN(c2nc3c([C@@H](C)Nc4ccccc4C(=O)O)cc(C)cn3c(=O)c2C#N)CC1(F)F] | 2-(((1R)-1-(3-cyano-2-(3,3- difluoro-4-methoxypiperidin-1-yl)- 7-methyl-4-oxo- 4H-pyrido[1,2-a]pyrimidin-9- yl)ethyl)amino)benzoic acid |
| 11-68 | 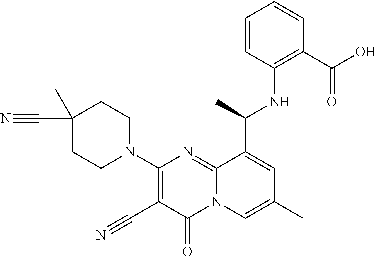 [SMILES: Cc1cc([C@@H](C)Nc2ccccc2C(=O)O)c2nc(N3CCC(C)(C#N)CC3)c(C#N)c(=O)n2c1] | (R)-2-((1-(3-cyano-2-(4-cyano-4- methylpiperidin-1-yl)-7-methyl-4- oxo-4H-pyrido[1,2-a]pyrimidin-9- yl)ethyl)amino)benzoic acid |
| 11-69 | 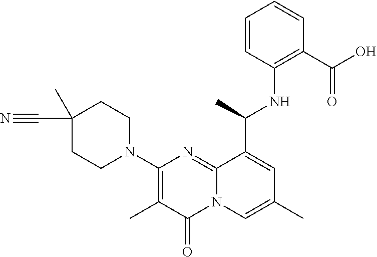 [SMILES: Cc1cc([C@@H](C)Nc2ccccc2C(=O)O)c2nc(N3CCC(C)(C#N)CC3)c(C)c(=O)n2c1] | (R)-2-((1-(2-(4-cyano-4- methylpiperidin-1-yl)-3,7- dimethyl-4-oxo- 4H-pyrido[1,2-a]pyrimidin-9- yl)ethyl)amino)benzoic acid |
| 11-70 | 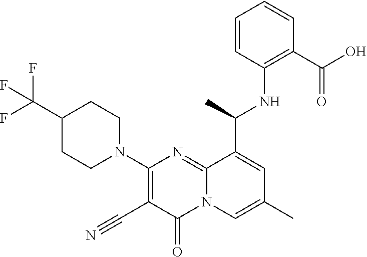 [SMILES: Cc1cc([C@@H](C)Nc2ccccc2C(=O)O)c2nc(N3CCC(C(F)(F)F)CC3)c(C#N)c(=O)n2c1] | (R)-2-((1-(3-cyano-7-methyl-4- oxo-2-(4- (trifluoromethyl)piperidin-1-yl)- 4H-pyrido[1,2-a]pyrimidin-9- yl)ethyl)amino)benzoic acid |
| 11-71 | 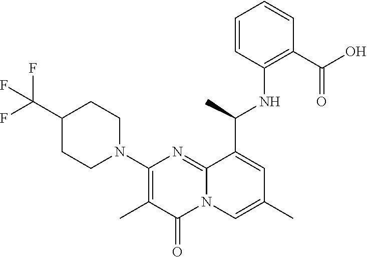 [SMILES: Cc1cc([C@@H](C)Nc2ccccc2C(=O)O)c2nc(N3CCC(C(F)(F)F)CC3)c(C)c(=O)n2c1] | (R)-2-((1-(3,7-dimethyl-4-oxo-2- (4-(trifluoromethyl)piperidin-1-yl)- 4H-pyrido[1,2-a]pyrimidin-9- yl)ethyl)amino)benzoic acid |
| 11-72 | 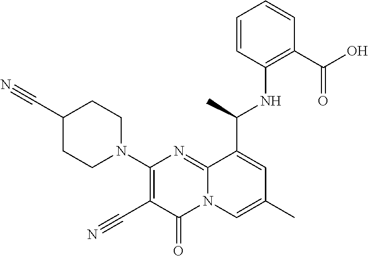 [SMILES: Cc1cc([C@@H](C)Nc2ccccc2C(=O)O)c2nc(N3CCC(C#N)CC3)c(C#N)c(=O)n2c1] | (R)-2-((1-(3-cyano-2-(4- cyanopiperidin-1-yl)-7-methyl-4- oxo-4H-pyrido[1,2-a]pyrimidin-9- yl)ethyl)amino)benzoic acid |
| 11-73 | 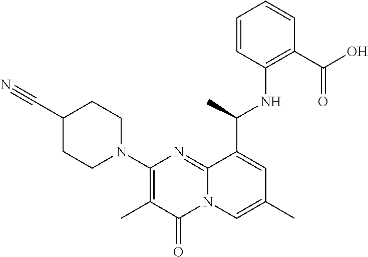 [SMILES: Cc1cc([C@@H](C)Nc2ccccc2C(=O)O)c2nc(N3CCC(C#N)CC3)c(C)c(=O)n2c1] | (R)-2-((1-(2-(4-cyanopiperidin-1- yl)-3,7-dimethyl-4-oxo- 4H-pyrido[1,2-a]pyrimidin-9- yl)ethyl)amino)benzoic acid |
| 11-74 | 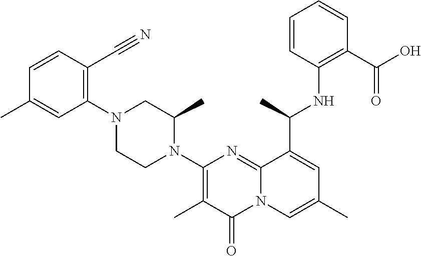 [SMILES: Cc1ccc(C#N)c(N2CCN(c3nc4c([C@@H](C)Nc5ccccc5C(=O)O)cc(C)cn4c(=O)c3C)[C@H](C)C2)c1] | 2-(((R)-1-(2-((R)-4-(2-cyano-5- methylphenyl)-2- methylpiperazin-1-yl)-3,7- dimethyl-4-oxo- 4H-pyrido[1,2-a]pyrimidin-9- yl)ethyl)amino)benzoic acid |
| 11-75 | 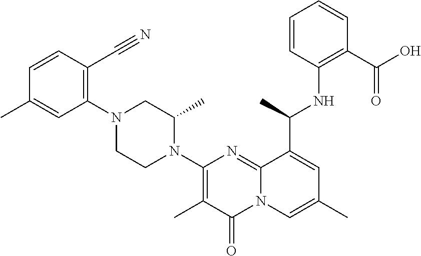 [SMILES: Cc1ccc(C#N)c(N2CCN(c3nc4c([C@@H](C)Nc5ccccc5C(=O)O)cc(C)cn4c(=O)c3C)[C@@H](C)C2)c1] | 2-(((R)-1-(2-((S)-4-(2-cyano-5- methylphenyl)-2- methylpiperazin-1-yl)-3,7- dimethyl-4-oxo- 4H-pyrido[1,2-a]pyrimidin-9- yl)ethyl)amino)benzoic acid |
| 11-76 | 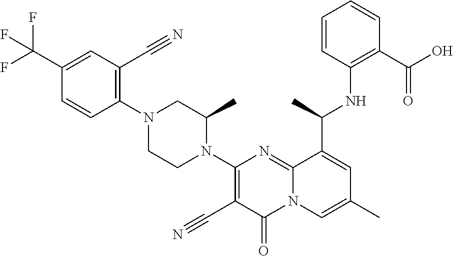 [SMILES: Cc1cc([C@@H](C)Nc2ccccc2C(=O)O)c2nc(N3CCN(c4ccc(C(F)(F)F)cc4C#N)C[C@H]3C)c(C#N)c(=O)n2c1] | 2-(((R)-1-(3-cyano-2-((R)-4-(2- cyano-4-(trifluoromethyl)phenyl)- 2-methylpiperazin-1-yl)-7-methyl- 4-oxo-4H-pyrido[1,2-a]pyrimidin-9- yl)ethyl)amino)benzoic acid |
| 11-77 | 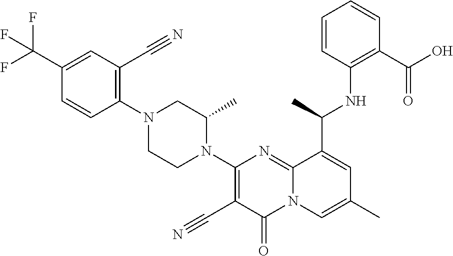 [SMILES: Cc1cc([C@@H](C)Nc2ccccc2C(=O)O)c2nc(N3CCN(c4ccc(C(F)(F)F)cc4C#N)C[C@@H]3C)c(C#N)c(=O)n2c1] | 2-(((R)-1-(3-cyano-2-((S)-4-(2- cyano-4-(trifluoromethyl)phenyl)- 2-methylpiperazin-1-yl)-7-methyl- 4-oxo-4H-pyrido[1,2-a]pyrimidin-9- yl)ethyl)amino)benzoic acid |
| 11-78 | 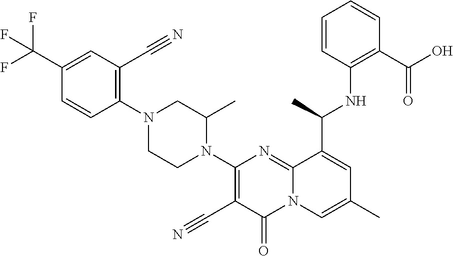 [SMILES: Cc1cc([C@@H](C)Nc2ccccc2C(=O)O)c2nc(N3CCN(c4ccc(C(F)(F)F)cc4C#N)CC3C)c(C#N)c(=O)n2c1] | 2-(((R)-1-(3-cyano-2-(4-(2- cyano-4-(trifluoromethyl)phenyl)- 2-methylpiperazin-1-yl)-7-methyl- 4-oxo-4H-pyrido[1,2-a]pyrimidin-9- yl)ethyl)amino)benzoic acid |
| 11-79 | 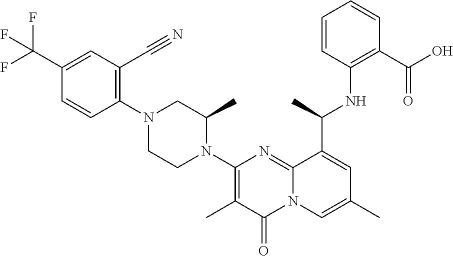 [SMILES: Cc1cc([C@@H](C)Nc2ccccc2C(=O)O)c2nc(N3CCN(c4ccc(C(F)(F)F)cc4C#N)C[C@H]3C)c(C)c(=O)n2c1] | 2-(((R)-1-(2-((R)-4-(2-cyano-4- (trifluoromethyl)phenyl)-2- methylpiperazin-1-yl)-3,7- dimethyl-4-oxo- 4H-pyrido[1,2-a]pyrimidin-9- yl)ethyl)amino)benzoic acid |
| 11-80 | 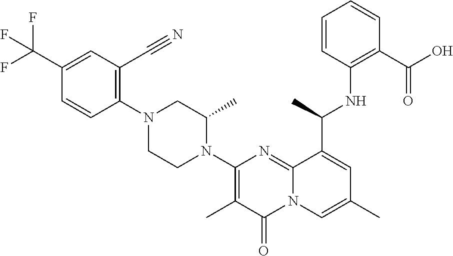 [SMILES: Cc1cc([C@@H](C)Nc2ccccc2C(=O)O)c2nc(N3CCN(c4ccc(C(F)(F)F)cc4C#N)C[C@@H]3C)c(C)c(=O)n2c1] | 2-(((R)-1-(2-((S)-4-(2-cyano-4- (trifluoromethyl)phenyl)-2- methylpiperazin-1-yl)-3,7- dimethyl-4-oxo- 4H-pyrido[1,2-a]pyrimidin-9- yl)ethyl)amino)benzoic acid |
| 11-81 | 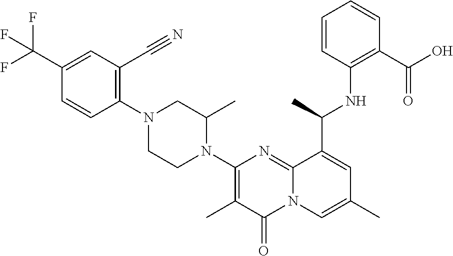 [SMILES: Cc1cc([C@@H](C)Nc2ccccc2C(=O)O)c2nc(N3CCN(c4ccc(C(F)(F)F)cc4C#N)CC3C)c(C)c(=O)n2c1] | 2-(((1R)-1-(2-(4-(2-cyano-4- (trifluoromethyl)phenyl)-2- methylpiperazin-1-yl)-3,7- dimethyl-4-oxo- 4H-pyrido[1,2-a]pyrimidin-9- yl)ethyl)amino)benzoic acid |
| 11-82 | 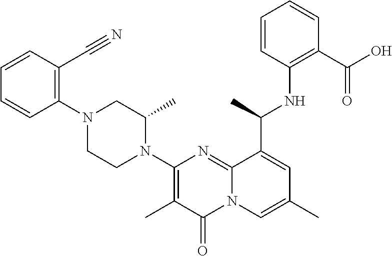 [SMILES: Cc1cc([C@@H](C)Nc2ccccc2C(=O)O)c2nc(N3CCN(c4ccccc4C#N)C[C@@H]3C)c(C)c(=O)n2c1] | 2-(((R)-1-(2-((S)-4-(2- cyanophenyl)-2-methylpiperazin- 1-yl)-3,7-dimethyl-4-oxo- 4H-pyrido[1,2-a]pyrimidin-9- yl)ethyl)amino)benzoic acid |
| 11-83 | 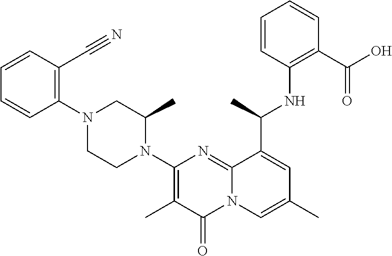 [SMILES: Cc1cc([C@@H](C)Nc2ccccc2C(=O)O)c2nc(N3CCN(c4ccccc4C#N)C[C@H]3C)c(C)c(=O)n2c1] | 2-(((R)-1-(2-((R)-4-(2- cyanophenyl)-2-methylpiperazin- 1-yl)-3,7-dimethyl-4-oxo- 4H-pyrido[1,2-a]pyrimidin-9- yl)ethyl)amino)benzoic acid |
| 11-84 | 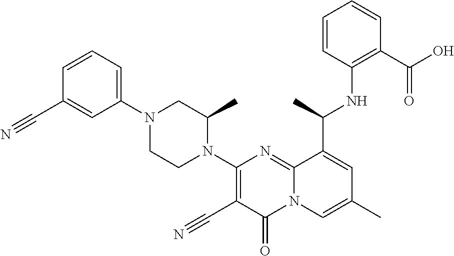 [SMILES: Cc1cc([C@@H](C)Nc2ccccc2C(=O)O)c2nc(N3CCN(c4cccc(C#N)c4)C[C@H]3C)c(C#N)c(=O)n2c1] | 2-(((R)-1-(3-cyano-2-((R)-4-(3- cyanophenyl)-2-methylpiperazin- 1-yl)-7-methyl-4-oxo- 4H-pyrido[1,2-a]pyrimidin-9- yl)ethyl)amino)benzoic acid |
| 11-85 | 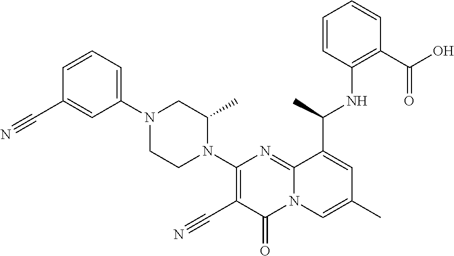 [SMILES: Cc1cc([C@@H](C)Nc2ccccc2C(=O)O)c2nc(N3CCN(c4cccc(C#N)c4)C[C@@H]3C)c(C#N)c(=O)n2c1] | 2-(((R)-1-(3-cyano-2-((S)-4-(3- cyanophenyl)-2-methylpiperazin- 1-yl)-7-methyl-4-oxo- 4H-pyrido[1,2-a]pyrimidin-9- yl)ethyl)amino)benzoic acid |
| 11-86 | 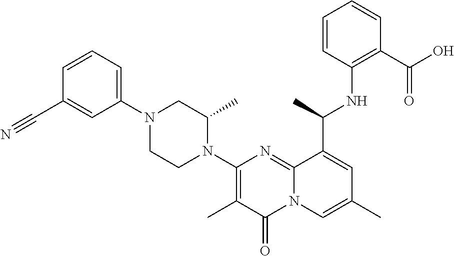 [SMILES: Cc1cc([C@@H](C)Nc2ccccc2C(=O)O)c2nc(N3CCN(c4cccc(C#N)c4)C[C@@H]3C)c(C)c(=O)n2c1] | 2-(((R)-1-(2-((S)-4-(3- cyanophenyl)-2-methylpiperazin- 1-yl)-3,7-dimethyl-4-oxo- 4H-pyrido[1,2-a]pyrimidin-9- yl)ethyl)amino)benzoic acid |
| 11-87 | 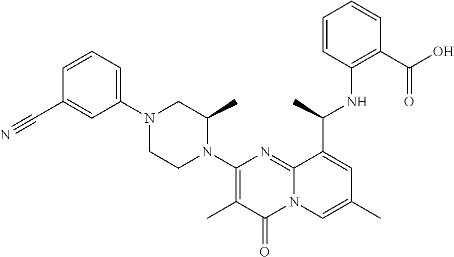 [SMILES: Cc1cc([C@@H](C)Nc2ccccc2C(=O)O)c2nc(N3CCN(c4cccc(C#N)c4)C[C@H]3C)c(C)c(=O)n2c1] | 2-(((R)-1-(2-((R)-4-(3- cyanophenyl)-2-methylpiperazin- 1-yl)-3,7-dimethyl-4-oxo- 4H-pyrido[1,2-a]pyrimidin-9- yl)ethyl)amino)benzoic acid |
| 11-88 | 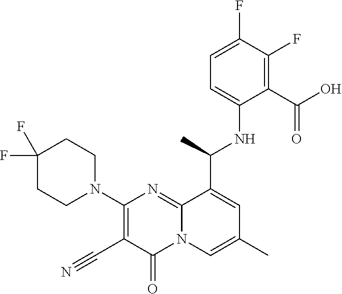 [SMILES: Cc1cc([C@@H](C)Nc2ccc(F)c(F)c2C(=O)O)c2nc(N3CCC(F)(F)CC3)c(C#N)c(=O)n2c1] | (R)-6-((1-(3-cyano-2-(4,4- difluoropiperidin-1-yl)-7-methyl- 4-oxo-4H-pyrido[1,2-a]pyrimidin- 9-yl)ethyl)amino)-2,3- difluorobenzoic acid |
| 11-89 | 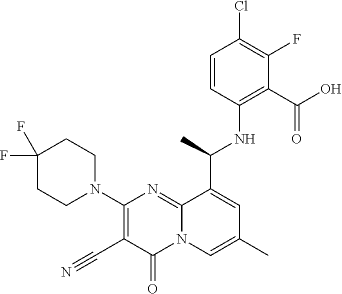 [SMILES: Cc1cc([C@@H](C)Nc2ccc(Cl)c(F)c2C(=O)O)c2nc(N3CCC(F)(F)CC3)c(C#N)c(=O)n2c1] | (R)-3-chloro-6-((1-(3-cyano-2- (4,4-difluoropiperidin-1-yl)-7- methyl-4-oxo-4H-pyrido[1,2- a]pyrimidin-9-yl)ethyl)amino)-2- fluorobenzoic acid |
| 11-90 | 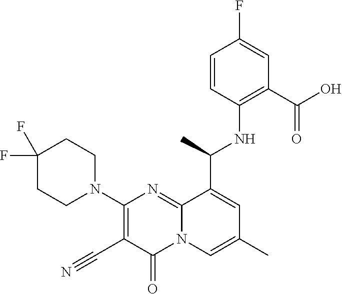 [SMILES: Cc1cc([C@@H](C)Nc2ccc(F)cc2C(=O)O)c2nc(N3CCC(F)(F)CC3)c(C#N)c(=O)n2c1] | (R)-2-((1-(3-cyano-2-(4,4- difluoropiperidin-1-yl)-7-methyl- 4-oxo-4H-pyrido[1,2-a]pyrimidin- 9-yl)ethyl)amino)-5-fluorobenzoic acid |
| 11-91 | 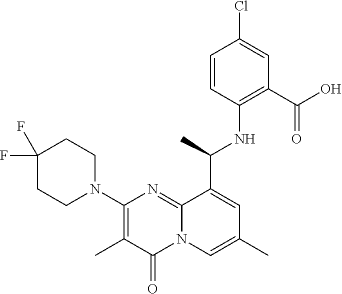 [SMILES: Cc1cc([C@@H](C)Nc2ccc(Cl)cc2C(=O)O)c2nc(N3CCC(F)(F)CC3)c(C)c(=O)n2c1] | (R)-5-chloro-2-((1-(2-(4,4- difluoropiperidin-1-yl)-3,7- dimethyl-4-oxo- 4H-pyrido[1,2-a]pyrimidin-9- yl)ethyl)amino)benzoic acid |
| 11-92 | 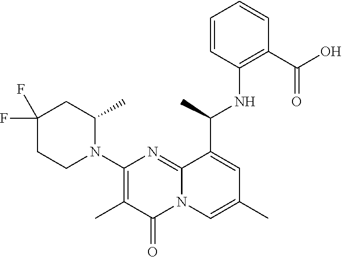 [SMILES: Cc1cc([C@@H](C)Nc2ccccc2C(=O)O)c2nc(N3CCC(F)(F)C[C@@H]3C)c(C)c(=O)n2c1] | 2-(((R)-1-(2-((S)-4,4-difluoro-2- methylpiperidin-1-yl)-3,7- dimethyl-4-oxo- 4H-pyrido[1,2-a]pyrimidin-9- yl)ethyl)amino)benzoic acid |
| 11-93 | 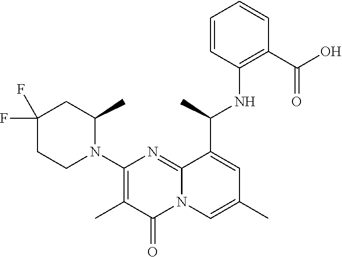 [SMILES: Cc1cc([C@@H](C)Nc2ccccc2C(=O)O)c2nc(N3CCC(F)(F)C[C@H]3C)c(C)c(=O)n2c1] | 2-(((R)-1-(2-((R)-4,4-difluoro-2- methylpiperidin-1-yl)-3,7- dimethyl-4-oxo- 4H-pyrido[1,2-a]pyrimidin-9- yl)ethyl)amino)benzoic acid |
| 11-94 | 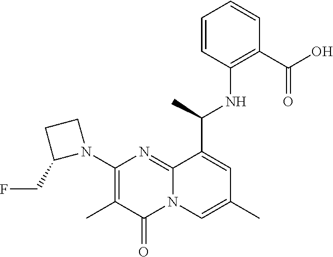 [SMILES: Cc1cc([C@@H](C)Nc2ccccc2C(=O)O)c2nc(N3CC[C@H]3CF)c(C)c(=O)n2c1] | 2-(((R)-1-(2-((S)-2- (fluoromethyl)azetidin-1-yl)-3,7- dimethyl-4-oxo- 4H-pyrido[1,2-a]pyrimidin-9- yl)ethyl)amino)benzoic acid |
| 11-95 | 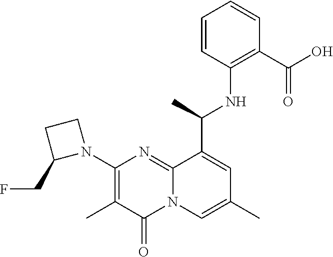 [SMILES: Cc1cc([C@@H](C)Nc2ccccc2C(=O)O)c2nc(N3CC[C@@H]3CF)c(C)c(=O)n2c1] | 2-(((R)-1-(2-((R)-2- (fluoromethyl)azetidin-1-yl)-3,7- dimethyl-4-oxo- 4H-pyrido[1,2-a]pyrimidin-9- yl)ethyl)amino)benzoic acid |
| 11-96 | 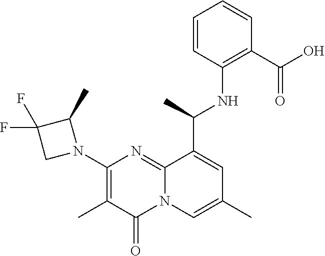 [SMILES: Cc1cc([C@@H](C)Nc2ccccc2C(=O)O)c2nc(N3CC(F)(F)[C@H]3C)c(C)c(=O)n2c1] | 2-(((R)-1-(2-((R)-3,3-difluoro-2- methylazetidin-1-yl)-3,7- dimethyl-4-oxo- 4H-pyrido[1,2-a]pyrimidin-9- yl)ethyl)amino)benzoic acid |
| 11-97 | 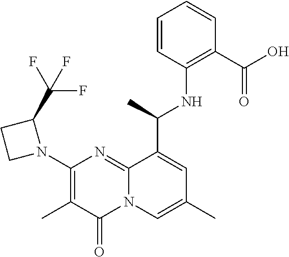 [SMILES: Cc1cc([C@@H](C)Nc2ccccc2C(=O)O)c2nc(N3CC[C@H]3C(F)(F)F)c(C)c(=O)n2c1] | 2-(((R)-1-(3,7-dimethyl-4-oxo-2- ((S)-2-(trifluoromethyl)azetidin-1- yl)-4H-pyrido[1,2-a]pyrimidin-9- yl)ethyl)amino)benzoic acid |
| 11-98 | 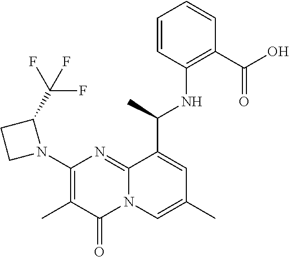 [SMILES: Cc1cc([C@@H](C)Nc2ccccc2C(=O)O)c2nc(N3CC[C@@H]3C(F)(F)F)c(C)c(=O)n2c1] | 2-(((R)-1-(3,7-dimethyl-4-oxo-2- ((R)-2-(trifluoromethyl)azetidin-1- yl)-4H-pyrido[1,2-a]pyrimidin-9- yl)ethyl)amino)benzoic acid |
| 11-99 | 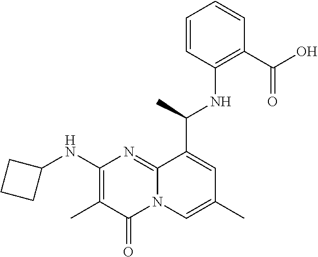 [SMILES: Cc1cc([C@@H](C)Nc2ccccc2C(=O)O)c2nc(NC3CCC3)c(C)c(=O)n2c1] | (R)-2-((1-(2-(cyclobutylamino)- 3,7-dimethyl-4-oxo- 4H-pyrido[1,2-a]pyrimidin-9- yl)ethyl)amino)benzoic acid |
| 11-100 | 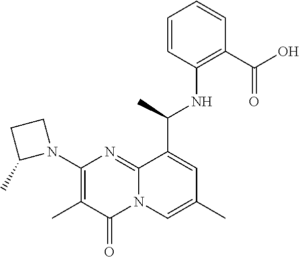 [SMILES: Cc1cc([C@@H](C)Nc2ccccc2C(=O)O)c2nc(N3CC[C@H]3C)c(C)c(=O)n2c1] | 2-(((R)-1-(3,7-dimethyl-2-((R)-2- methylazetidin-1-yl)-4-oxo- 4H-pyrido[1,2-a]pyrimidin-9- yl)ethyl)amino)benzoic acid |
| 11-101 | 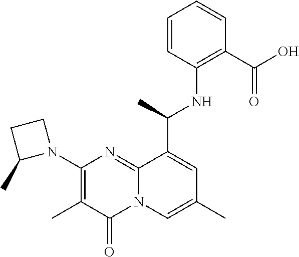 [SMILES: Cc1cc([C@@H](C)Nc2ccccc2C(=O)O)c2nc(N3CC[C@@H]3C)c(C)c(=O)n2c1] | 2-(((R)-1-(3,7-dimethyl-2-((S)-2- methylazetidin-1-yl)-4-oxo- 4H-pyrido[1,2-a]pyrimidin-9- yl)ethyl)amino)benzoic acid |
| 11-102 | 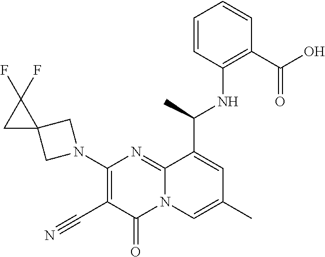 [SMILES: Cc1cc([C@@H](C)Nc2ccccc2C(=O)O)c2nc(N3CC4(C3)CC4(F)F)c(C#N)c(=O)n2c1] | (R)-2-((1-(3-cyano-2-(1,1- difluoro-5-azaspiro[2.3]hexan-5- yl)-7-methyl-4-oxo- 4H-pyrido[1,2-a]pyrimidin-9- yl)ethyl)amino)benzoic acid |
| 11-103 | 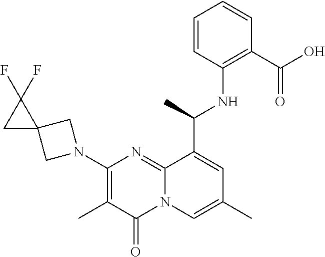 [SMILES: Cc1cc([C@@H](C)Nc2ccccc2C(=O)O)c2nc(N3CC4(C3)CC4(F)F)c(C)c(=O)n2c1] | (R)-2-((1-(2-(1,1-difluoro-5- azaspiro[2.3]hexan-5-yl)-3,7- dimethyl-4-oxo- 4H-pyrido[1,2-a]pyrimidin-9- yl)ethyl)amino)benzoic acid |
| 11-104 | 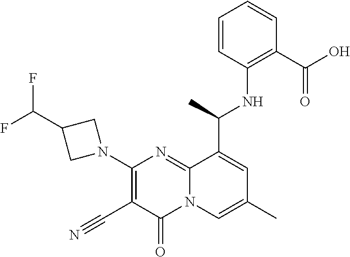 [SMILES: Cc1cc([C@@H](C)Nc2ccccc2C(=O)O)c2nc(N3CC(C(F)F)C3)c(C#N)c(=O)n2c1] | (R)-2-((1-(3-cyano-2-(3- (difluoromethyl)azetidin-1-yl)-7- methyl-4-oxo- 4H-pyrido[1,2-a]pyrimidin-9- yl)ethyl)amino)benzoic acid |
| 11-105 | 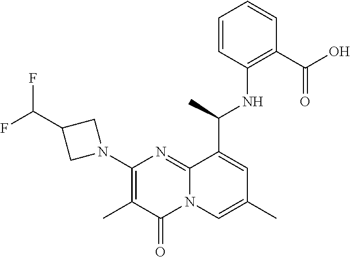 [SMILES: Cc1cc([C@@H](C)Nc2ccccc2C(=O)O)c2nc(N3CC(C(F)F)C3)c(C)c(=O)n2c1] | (R)-2-((1-(2-(3- (difluoromethyl)azetidin-1-yl)-3,7- dimethyl-4-oxo- 4H-pyrido[1,2-a]pyrimidin-9- yl)ethyl)amino)benzoic acid |
| 11-106 | 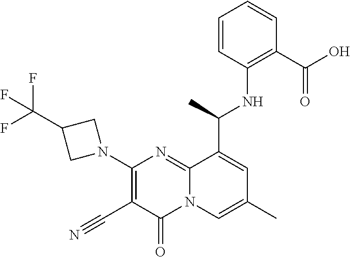 [SMILES: Cc1cc([C@@H](C)Nc2ccccc2C(=O)O)c2nc(N3CC(C(F)(F)F)C3)c(C#N)c(=O)n2c1] | (R)-2-((1-(3-cyano-7-methyl-4- oxo-2-(3- (trifluoromethyl)azetidin-1-yl)- 4H-pyrido[1,2-a]pyrimidin-9- yl)ethyl)amino)benzoic acid |
| 11-107 | 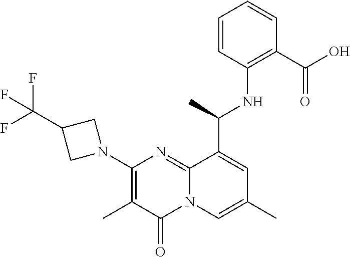 [SMILES: Cc1cc([C@@H](C)Nc2ccccc2C(=O)O)c2nc(N3CC(C(F)(F)F)C3)c(C)c(=O)n2c1] | (R)-2-((1-(3,7-dimethyl-4-oxo-2- (3-(trifluoromethyl)azetidin-1-yl)- 4H-pyrido[1,2-a]pyrimidin-9- yl)ethyl)amino)benzoic acid |
| 11-108 | 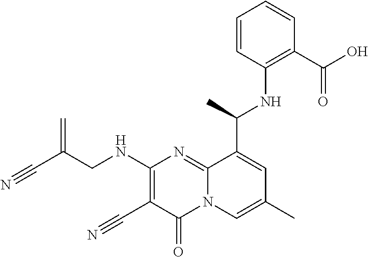 [SMILES: C=C(C#N)CNc1nc2c([C@@H](C)Nc3ccccc3C(=O)O)cc(C)cn2c(=O)c1C#N] | (R)-2-((1-(3-cyano-2-((2- cyanoallyl)amino)-7-methyl-4- oxo-4H-pyrido[1,2-a]pyrimidin-9- yl)ethyl)amino)benzoic acid |
| 11-109 | 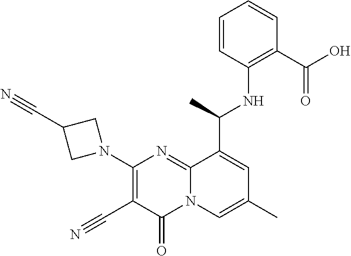 [SMILES: Cc1cc([C@@H](C)Nc2ccccc2C(=O)O)c2nc(N3CC(C#N)C3)c(C#N)c(=O)n2c1] | (R)-2-((1-(3-cyano-2-(3- cyanoazetidin-1-yl)-7-methyl-4- oxo-4H-pyrido[1,2-a]pyrimidin-9- yl)ethyl)amino)benzoic acid |
| 11-110 | 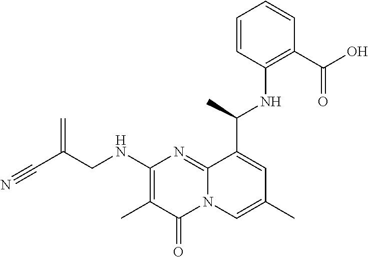 [SMILES: C=C(C#N)CNc1nc2c([C@@H](C)Nc3ccccc3C(=O)O)cc(C)cn2c(=O)c1C] | (R)-2-((1-(2-((2- cyanoallyl)amino)-3,7-dimethyl- 4-oxo-4H-pyrido[1,2-a]pyrimidin-9- yl)ethyl)amino)benzoic acid |
| 11-111 | 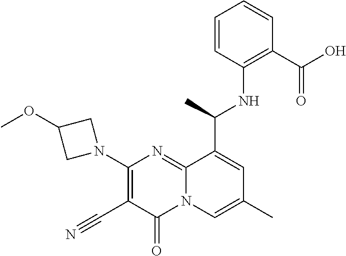 [SMILES: COC1CN(c2nc3c([C@@H](C)Nc4ccccc4C(=O)O)cc(C)cn3c(=O)c2C#N)C1] | (R)-2-((1-(3-cyano-2-(3- methoxyazetidin-1-yl)-7-methyl- 4-oxo-4H-pyrido[1,2-a]pyrimidin-9- yl)ethyl)amino)benzoic acid |
| 11-112 | 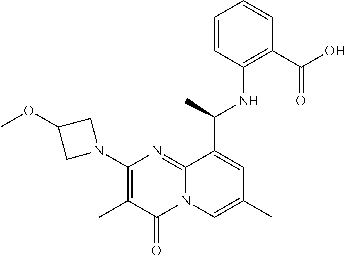 [SMILES: COC1CN(c2nc3c([C@@H](C)Nc4ccccc4C(=O)O)cc(C)cn3c(=O)c2C)C1] | (R)-2-((1-(2-(3-methoxyazetidin- 1-yl)-3,7-dimethyl-4-oxo- 4H-pyrido[1,2-a]pyrimidin-9- yl)ethyl)amino)benzoic acid |
| 11-113 | 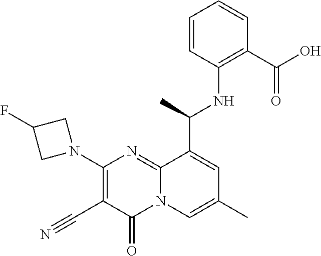 [SMILES: Cc1cc([C@@H](C)Nc2ccccc2C(=O)O)c2nc(N3CC(F)C3)c(C#N)c(=O)n2c1] | (R)-2-((1-(3-cyano-2-(3- fluoroazetidin-1-yl)-7-methyl-4- oxo-4H-pyrido[1,2-a]pyrimidin-9- yl)ethyl)amino)benzoic acid |
| 11-114 | 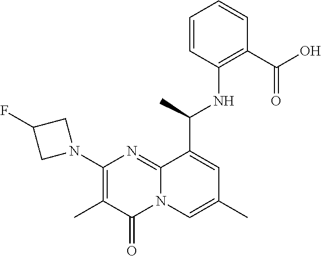 [SMILES: Cc1cc([C@@H](C)Nc2ccccc2C(=O)O)c2nc(N3CC(F)C3)c(C)c(=O)n2c1] | (R)-2-((1-(2-(3-fluoroazetidin-1- yl)-3,7-dimethyl-4-oxo- 4H-pyrido[1,2-a]pyrimidin-9- yl)ethyl)amino)benzoic acid |
| 11-115 | 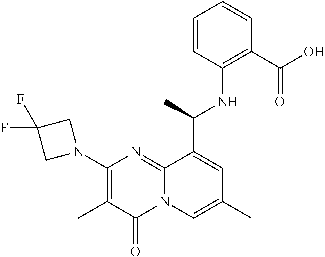 [SMILES: Cc1cc([C@@H](C)Nc2ccccc2C(=O)O)c2nc(N3CC(F)(F)C3)c(C)c(=O)n2c1] | (R)-2-((1-(2-(3,3-difluoroazetidin- 1-yl)-3,7-dimethyl-4-oxo- 4H-pyrido[1,2-a]pyrimidin-9- yl)ethyl)amino)benzoic acid |
| 11-116 | 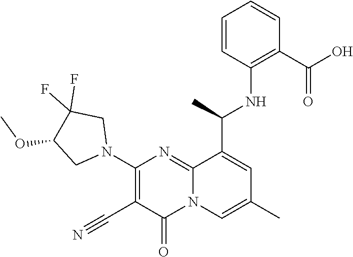 [SMILES: CO[C@H]1CN(c2nc3c([C@@H](C)Nc4ccccc4C(=O)O)cc(C)cn3c(=O)c2C#N)CC1(F)F] | 2-(((R)-1-(3-cyano-2-((S)-3,3- difluoro-4-methoxypyrrolidin-1- yl)-7-methyl-4-oxo- 4H-pyrido[1,2-a]pyrimidin-9- yl)ethyl)amino)benzoic acid |
| 11-117 | 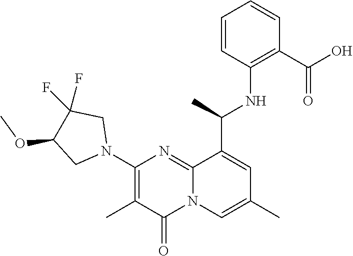 [SMILES: CO[C@@H]1CN(c2nc3c([C@@H](C)Nc4ccccc4C(=O)O)cc(C)cn3c(=O)c2C)CC1(F)F] | 2-(((R)-1-(2-((R)-3,3-difluoro-4- methoxypyrrolidin-1-yl)-3,7- dimethyl-4-oxo- 4H-pyrido[1,2-a]pyrimidin-9- yl)ethyl)amino)benzoic acid |
| 11-118 | 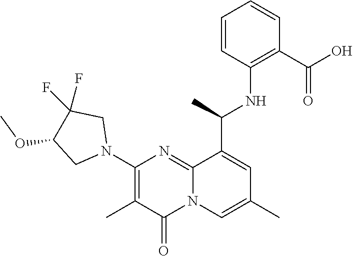 [SMILES: CO[C@H]1CN(c2nc3c([C@@H](C)Nc4ccccc4C(=O)O)cc(C)cn3c(=O)c2C)CC1(F)F] | 2-(((R)-1-(2-((S)-3,3-difluoro-4- methoxypyrrolidin-1-yl)-3,7- dimethyl-4-oxo- 4H-pyrido[1,2-a]pyrimidin-9- yl)ethyl)amino)benzoic acid |
| 11-119 | 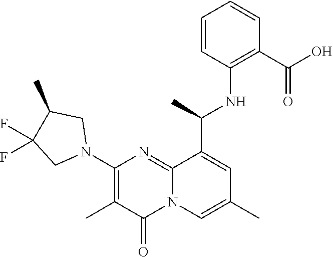 [SMILES: Cc1cc([C@@H](C)Nc2ccccc2C(=O)O)c2nc(N3C[C@H](C)C(F)(F)C3)c(C)c(=O)n2c1] | 2-(((R)-1-(2-((S)-3,3-difluoro-4- methylpyrrolidin-1-yl)-3,7- dimethyl-4-oxo- 4H-pyrido[1,2-a]pyrimidin-9- yl)ethyl)amino)benzoic acid |
| 11-120 | 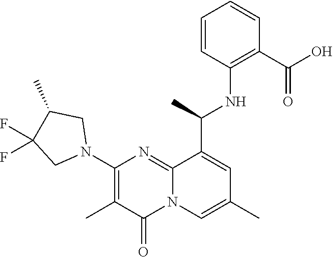 [SMILES: Cc1cc([C@@H](C)Nc2ccccc2C(=O)O)c2nc(N3C[C@@H](C)C(F)(F)C3)c(C)c(=O)n2c1] | 2-(((R)-1-(2-((R)-3,3-difluoro-4- methylpyrrolidin-1-yl)-3,7- dimethyl-4-oxo- 4H-pyrido[1,2-a]pyrimidin-9- yl)ethyl)amino)benzoic acid |
| 11-121 | 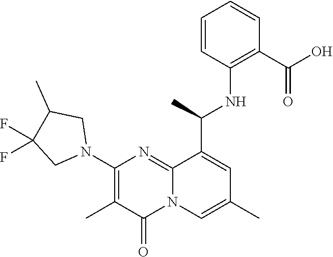 [SMILES: Cc1cc([C@@H](C)Nc2ccccc2C(=O)O)c2nc(N3CC(C)C(F)(F)C3)c(C)c(=O)n2c1] | 2-(((R)-1-(2-(3,3-difluoro-4- methylpyrrolidin-1-yl)-3,7- dimethyl-4-oxo- 4H-pyrido[1,2-a]pyrimidin-9- yl)ethyl)amino)benzoic acid |
| 11-122 | 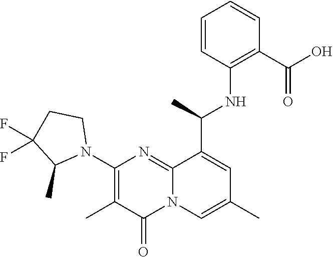 [SMILES: Cc1cc([C@@H](C)Nc2ccccc2C(=O)O)c2nc(N3CCC(F)(F)[C@@H]3C)c(C)c(=O)n2c1] | 2-(((R)-1-(2-((S)-3,3-difluoro-2- methylpyrrolidin-1-yl)-3,7- dimethyl-4-oxo- 4H-pyrido[1,2-a]pyrimidin-9- yl)ethyl)amino)benzoic acid |
| 11-123 | 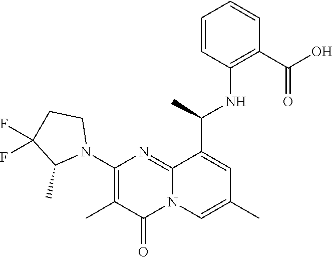 [SMILES: Cc1cc([C@@H](C)Nc2ccccc2C(=O)O)c2nc(N3CCC(F)(F)[C@H]3C)c(C)c(=O)n2c1] | 2-(((R)-1-(2-((R)-3,3-difluoro-2- methylpyrrolidin-1-yl)-3,7- dimethyl-4-oxo- 4H-pyrido[1,2-a]pyrimidin-9- yl)ethyl)amino)benzoic acid |
| 11-124 | 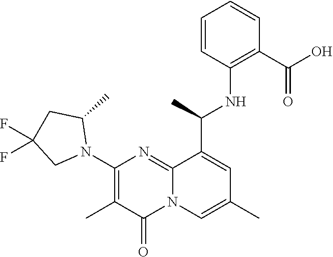 [SMILES: Cc1cc([C@@H](C)Nc2ccccc2C(=O)O)c2nc(N3CC(F)(F)C[C@@H]3C)c(C)c(=O)n2c1] | 2-(((R)-1-(3-cyano-2-((S)-4,4- difluoro-2-methylpyrrolidin-1-yl)- 7-methyl-4-oxo- 4H-pyrido[1,2-a]pyrimidin-9- yl)ethyl)amino)benzoic acid |
| 11-125 | 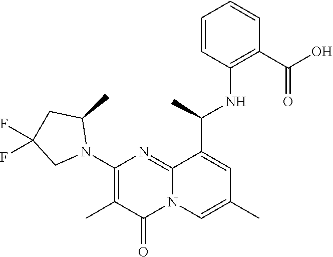 [SMILES: Cc1cc([C@@H](C)Nc2ccccc2C(=O)O)c2nc(N3CC(F)(F)C[C@H]3C)c(C)c(=O)n2c1] | 2-(((R)-1-(2-((R)-4,4-difluoro-2- methylpyrrolidin-1-yl)-3,7- dimethyl-4-oxo- 4H-pyrido[1,2-a]pyrimidin-9- yl)ethyl)amino)benzoic acid |
| 11-126 | 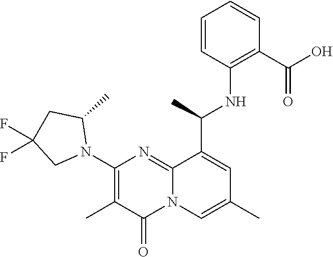 [SMILES: Cc1cc([C@@H](C)Nc2ccccc2C(=O)O)c2nc(N3CC(F)(F)C[C@@H]3C)c(C)c(=O)n2c1] | 2-(((R)-1-(2-((S)-4,4-difluoro-2- methylpyrrolidin-1-yl)-3,7- dimethyl-4-oxo- 4H-pyrido[1,2-a]pyrimidin-9- yl)ethyl)amino)benzoic acid |
| 11-127 | 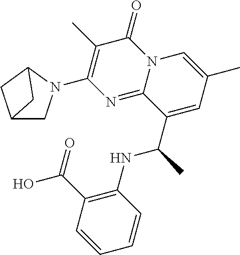 [SMILES: Cc1cc([C@@H](C)Nc2ccccc2C(=O)O)c2nc(N3CC4CC3C4)c(C)c(=O)n2c1] | (R)-2-((1-(2-(2- azabicyclo[2.1.1]hexan-2-yl)-3,7- dimethyl-4-oxo- 4H-pyrido[1,2-a]pyrimidin-9- yl)ethyl)amino)benzoic acid |
| 11-128 | 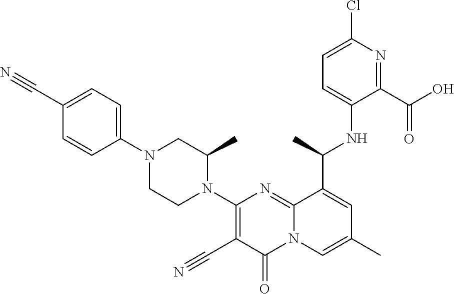 [SMILES: Cc1cc([C@@H](C)Nc2ccc(Cl)nc2C(=O)O)c2nc(N3CCN(c4ccc(C#N)cc4)C[C@H]3C)c(C#N)c(=O)n2c1] | 6-chloro-3-(((R)-1-(3-cyano-2- ((R)-4-(4-cyanophenyl)-2- methylpiperazin-1-yl)-7-methyl-4- oxo-4H-pyrido[1,2-a]pyrimidin-9- yl)ethyl)amino)picolinic acid |
| 11-129 | 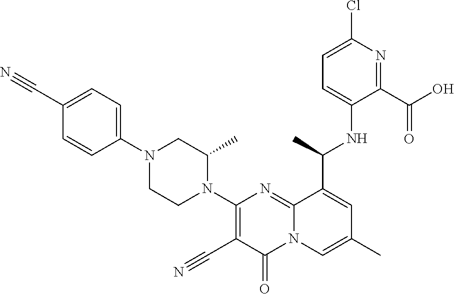 [SMILES: Cc1cc([C@@H](C)Nc2ccc(Cl)nc2C(=O)O)c2nc(N3CCN(c4ccc(C#N)cc4)C[C@@H]3C)c(C#N)c(=O)n2c1] | 6-chloro-3-(((R)-1-(3-cyano-2- ((S)-4-(4-cyanophenyl)-2- methylpiperazin-1-yl)-7-methyl-4- oxo-4H-pyrido[1,2-a]pyrimidin-9- yl)ethyl)amino)picolinic acid |
| 11-130 | 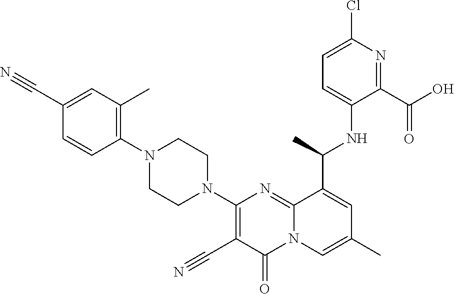 [SMILES: Cc1cc([C@@H](C)Nc2ccc(Cl)nc2C(=O)O)c2nc(N3CCN(c4ccc(C#N)cc4C)CC3)c(C#N)c(=O)n2c1] | 6-chloro-3-(((R)-1-(3-cyano-2- ((S)-4-(4-fluorophenyl)-2- methylpiperazin-1-yl)-7-methyl-4- oxo-4H-pyrido[1,2-a]pyrimidin-9- yl)ethyl)amino)picolinic acid |
| 11-131 | 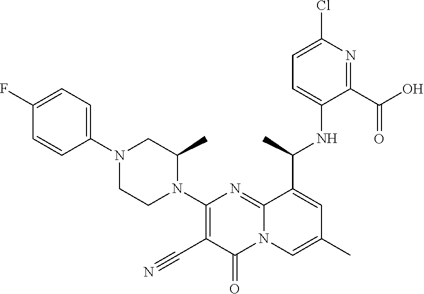 [SMILES: Cc1cc([C@@H](C)Nc2ccc(Cl)nc2C(=O)O)c2nc(N3CCN(c4ccc(F)cc4)C[C@H]3C)c(C#N)c(=O)n2c1] | 6-chloro-3-(((R)-1-(3-cyano-2- ((R)-4-(4-fluorophenyl)-2- methylpiperazin-1-yl)-7-methyl-4- oxo-4H-pyrido[1,2-a]pyrimidin-9- yl)ethyl)amino)picolinic acid |
| 11-132 | 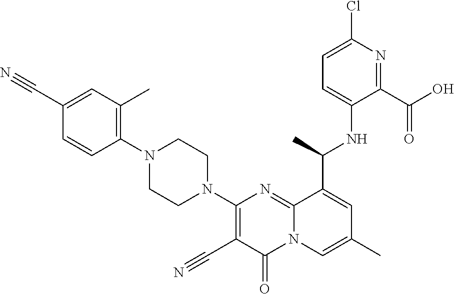 [SMILES: Cc1cc([C@@H](C)Nc2ccc(Cl)nc2C(=O)O)c2nc(N3CCN(c4ccc(C#N)cc4C)CC3)c(C#N)c(=O)n2c1] | (R)-6-chloro-3-((1-(3-cyano-2-(4- (4-cyano-2- methylphenyl)piperazin-1-yl)-7- methyl-4-oxo- 4H-pyrido[1,2-a]pyrimidin-9- yl)ethyl)amino)picolinic acid |
| 11-133 | 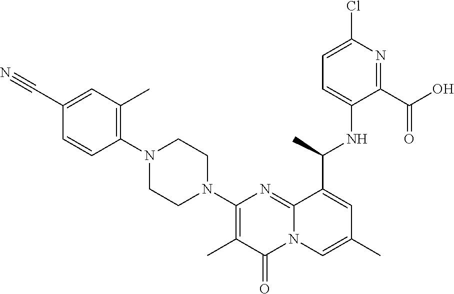 [SMILES: Cc1cc([C@@H](C)Nc2ccc(Cl)nc2C(=O)O)c2nc(N3CCN(c4ccc(C#N)cc4C)CC3)c(C)c(=O)n2c1] | (R)-6-chloro-3-((1-(2-(4-(4- cyano-2-methylphenyl)piperazin- 1-yl)-3,7-dimethyl-4-oxo- 4H-pyrido[1,2-a]pyrimidin-9- yl)ethyl)amino)picolinic acid |
| 11-134 | 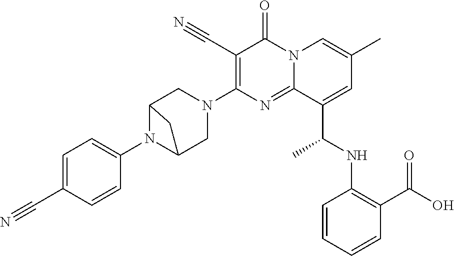 [SMILES: Cc1cc([C@@H](C)Nc2ccccc2C(=O)O)c2nc(N3CC4CC(C3)N4c3ccc(C#N)cc3)c(C#N)c(=O)n2c1] | 2-(((R)-1-(3-cyano-2-(6-(4- cyanophenyl)-3,6- diazabicyclo[3.1.1]heptan-3-yl)- 7-methyl-4-oxo- 4H-pyrido[1,2-a]pyrimidin-9- yl)ethyl)amino)benzoic acid |
| 11-135 | 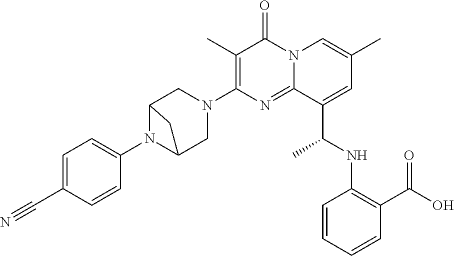 [SMILES: Cc1cc([C@@H](C)Nc2ccccc2C(=O)O)c2nc(N3CC4CC(C3)N4c3ccc(C#N)cc3)c(C)c(=O)n2c1] | 2-(((R)-1-(2-(6-(4-cyanophenyl)- 3,6-diazabicyclo[3.1.1]heptan-3- yl)-3,7-dimethyl-4-oxo- 4H-pyrido[1,2-a]pyrimidin-9- yl)ethyl)amino)benzoic acid |
| 11-136 | 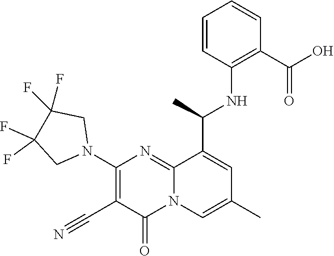 [SMILES: Cc1cc([C@@H](C)Nc2ccccc2C(=O)O)c2nc(N3CC(F)(F)C(F)(F)C3)c(C#N)c(=O)n2c1] | (R)-2-((1-(3-cyano-7-methyl-4- oxo-2-(3,3,4,4- tetrafluoropyrrolidin-1-yl)- 4H-pyrido[1,2-a]pyrimidin-9- yl)ethyl)amino)benzoic acid |
| 11-137 | 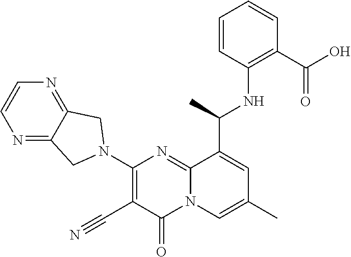 [SMILES: Cc1cc([C@@H](C)Nc2ccccc2C(=O)O)c2nc(N3Cc4nccnc4C3)c(C#N)c(=O)n2c1] | (R)-2-((1-(3-cyano-2-(5,7- dihydro-6H-pyrrolo[3,4-b]pyrazin- 6-yl)-7-methyl-4-oxo- 4H-pyrido[1,2-a]pyrimidin-9- yl)ethyl)amino)benzoic acid |
| 11-138 | 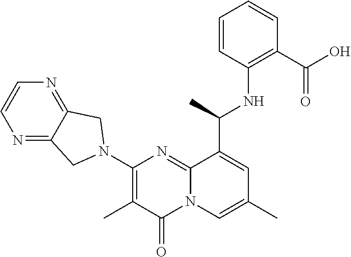 [SMILES: Cc1cc([C@@H](C)Nc2ccccc2C(=O)O)c2nc(N3Cc4nccnc4C3)c(C)c(=O)n2c1] | (R)-2-((1-(2-(5,7-dihydro-6H- pyrrolo[3,4-b]pyrazin-6-yl)-3,7- dimethyl-4-oxo- 4H-pyrido[1,2-a]pyrimidin-9- yl)ethyl)amino)benzoic acid |
| 11-139 | 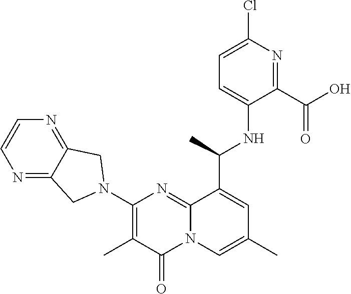 [SMILES: Cc1cc([C@@H](C)Nc2ccc(Cl)nc2C(=O)O)c2nc(N3Cc4nccnc4C3)c(C)c(=O)n2c1] | (R)-6-chloro-3-((1-(2-(5,7- dihydro-6H-pyrrolo[3,4-b]pyrazin- 6-yl)-3,7-dimethyl-4-oxo- 4H-pyrido[1,2-a]pyrimidin-9- yl)ethyl)amino)picolinic acid |
| 11-140 | 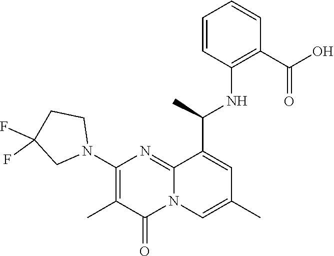 [SMILES: Cc1cc([C@@H](C)Nc2ccccc2C(=O)O)c2nc(N3CCC(F)(F)C3)c(C)c(=O)n2c1] | (R)-2-((1-(2-(3,3- difluoropyrrolidin-1-yl)-3,7- dimethyl-4-oxo- 4H-pyrido[1,2-a]pyrimidin-9- yl)ethyl)amino)benzoic acid |
| 11-141 | 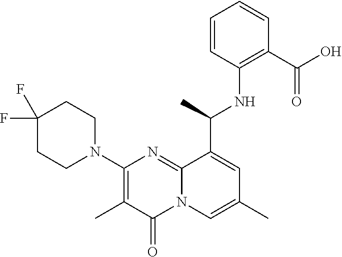 [SMILES: Cc1cc([C@@H](C)Nc2ccccc2C(=O)O)c2nc(N3CCC(F)(F)CC3)c(C)c(=O)n2c1] | (R)-2-((1-(2-(4,4- difluorocyclohexyl)-3,7-dimethyl- 4-oxo-4H-pyrido[1,2-a]pyrimidin-9- yl)ethyl)amino)benzoic acid |
| 11-142 | 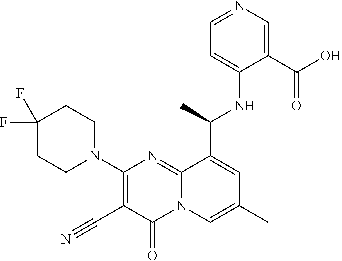 [SMILES: Cc1cc([C@@H](C)Nc2ccncc2C(=O)O)c2nc(N3CCC(F)(F)CC3)c(C#N)c(=O)n2c1] | (R)-4-((1-(3-cyano-2-(4,4- difluoropiperidin-1-yl)-7-methyl- 4-oxo-4H-pyrido[1,2-a]pyrimidin- 9-yl)ethyl)amino)nicotinic acid |
| 11-143 | 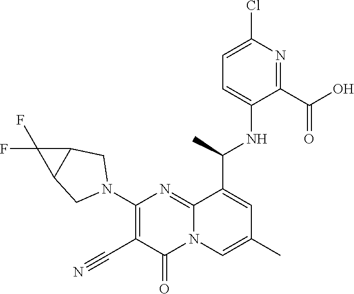 [SMILES: Cc1cc([C@@H](C)Nc2ccc(Cl)nc2C(=O)O)c2nc(N3CC4C(C3)C4(F)F)c(C#N)c(=O)n2c1] | 6-chloro-3-(((R)-1-(3-cyano-2- (6,6-difluoro-3- azabicyclo[3.1.0]hexan-3-yl)-7- methyl-4-oxo-4H-pyrido[1,2- a]pyrimidin-9- yl)ethyl)amino)picolinic acid |
| 11-144 | 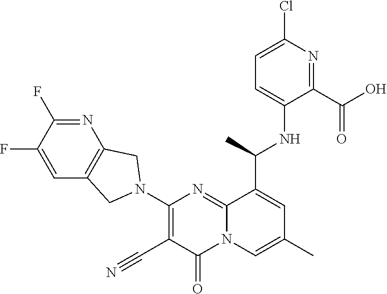 [SMILES: Cc1cc([C@@H](C)Nc2ccc(Cl)nc2C(=O)O)c2nc(N3Cc4cc(F)c(F)nc4C3)c(C#N)c(=O)n2c1] | (R)-6-chloro-3-((1-(3-cyano-2- (5,6-difluoroisoindolin-2-yl)-7- methyl-4-oxo-4H-pyrido[1,2-a] pyrimidin-9- yl)ethyl)amino)picolinic acid |
| 11-145 | 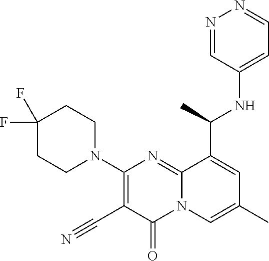 [SMILES: Cc1cc([C@@H](C)Nc2ccnnc2)c2nc(N3CCC(F)(F)CC3)c(C#N)c(=O)n2c1] | (R)-2-(4,4-difluoropiperidin-1-yl)- 7-methyl-4-oxo-9-(1-(pyridazin-4- ylamino)ethyl)-4H-pyrido[1,2- a]pyrimidine-3-carbonitrile |
| 11-146 | 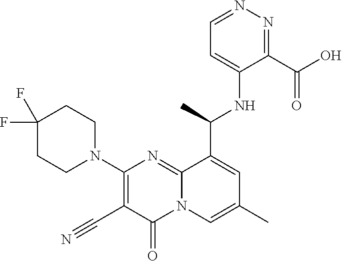 [SMILES: Cc1cc([C@@H](C)Nc2ccnnc2C(=O)O)c2nc(N3CCC(F)(F)CC3)c(C#N)c(=O)n2c1] | (R)-4-((1-(3-cyano-2-(4,4- difluoropiperidin-1-yl)-7-methyl- 4-oxo-4H-pyrido[1,2-a]pyrimidin- 9-yl)ethyl)amino)pyridazine-3- carboxylic acid |
| 11-147 | 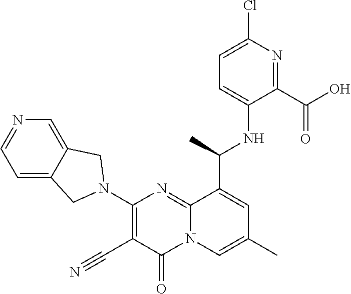 [SMILES: Cc1cc([C@@H](C)Nc2ccc(Cl)nc2C(=O)O)c2nc(N3Cc4ccncc4C3)c(C#N)c(=O)n2c1] | (R)-6-chloro-3-((1-(3-cyano-2- (1,3-dihydro-2H-pyrrolo[3,4- c]pyridin-2-yl)-7-methyl-4-oxo- 4H-pyrido[1,2-a]pyrimidin-9- yl)ethyl)amino)picolinic acid |
| 11-148 | 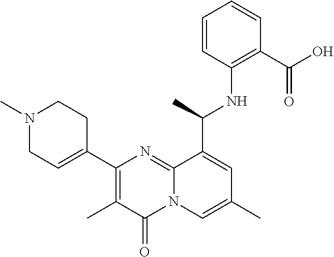 [SMILES: Cc1cc([C@@H](C)Nc2ccccc2C(=O)O)c2nc(C3=CCN(C)CC3)c(C)c(=O)n2c1] | (R)-2-((1-(3,7-dimethyl-2-(1- methyl-1,2,3,6-tetrahydropyridin- 4-yl)-4-oxo-4H-pyrido[1,2- a]pyrimidin-9- yl)ethyl)amino)benzoic acid |
| 11-149 | 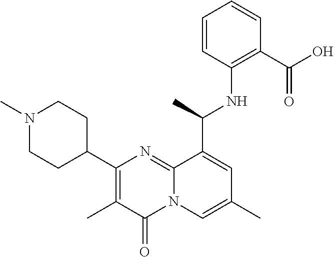 [SMILES: Cc1cc([C@@H](C)Nc2ccccc2C(=O)O)c2nc(C3CCN(C)CC3)c(C)c(=O)n2c1] | (R)-2-((1-(3,7-dimethyl-2-(1- methylpiperidin-4-yl)-4-oxo- 4H-pyrido[1,2-a]pyrimidin-9- yl)ethyl)amino)benzoic acid |
| 11-150 | 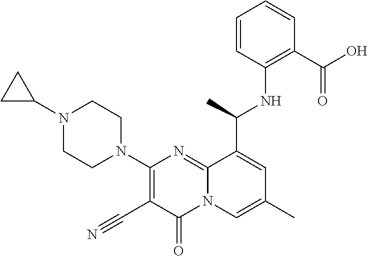 [SMILES: Cc1cc([C@@H](C)Nc2ccccc2C(=O)O)c2nc(N3CCN(C4CC4)CC3)c(C#N)c(=O)n2c1] | (R)-2-((1-(3-cyano-2-(4- cyclopropylpiperazin-1-yl)-7- methyl-4-oxo- 4H-pyrido[1,2-a]pyrimidin-9- yl)ethyl)amino)benzoic acid |
| 11-151 | 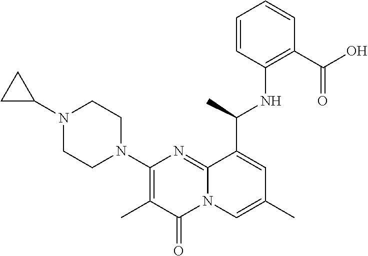 [SMILES: Cc1cc([C@@H](C)Nc2ccccc2C(=O)O)c2nc(N3CCN(C4CC4)CC3)c(C)c(=O)n2c1] | (R)-2-((1-(2-(4- cyclopropylpiperazin-1-yl)-3,7- dimethyl-4-oxo- 4H-pyrido[1,2-a]pyrimidin-9- yl)ethyl)amino)benzoic acid |
| 11-152 | 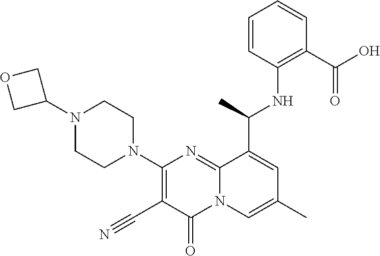 [SMILES: Cc1cc([C@@H](C)Nc2ccccc2C(=O)O)c2nc(N3CCN(C4COC4)CC3)c(C#N)c(=O)n2c1] | (R)-2-((1-(3-cyano-7-methyl-2-(4- (oxetan-3-yl)piperazin-1-yl)-4- oxo-4H-pyrido[1,2-a]pyrimidin-9- yl)ethyl)amino)benzoic acid |
| 11-153 | 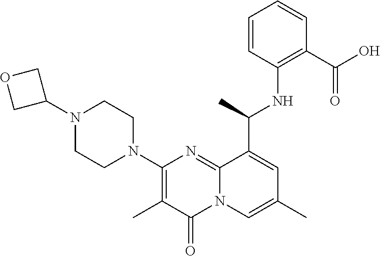 [SMILES: Cc1cc([C@@H](C)Nc2ccccc2C(=O)O)c2nc(N3CCN(C4COC4)CC3)c(C)c(=O)n2c1] | (R)-2-((1-(3,7-dimethyl-2-(4- (oxetan-3-yl)piperazin-1-yl)-4- oxo-4H-pyrido[1,2-a]pyrimidin-9- yl)ethyl)amino)benzoic acid |
| 11-154 | 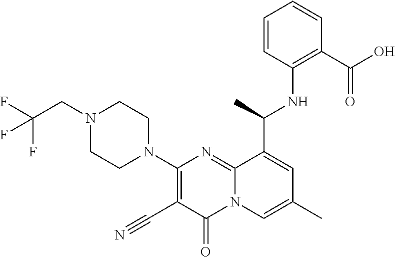 [SMILES: Cc1cc([C@@H](C)Nc2ccccc2C(=O)O)c2nc(N3CCN(CC(F)(F)F)CC3)c(C#N)c(=O)n2c1] | (R)-2-((1-(3-cyano-7-methyl-4- oxo-2-(4-(2,2,2- trifluoroethyl)piperazin-1-yl)- 4H-pyrido[1,2-a]pyrimidin-9- yl)ethyl)amino)benzoic acid |
| 11-155 | 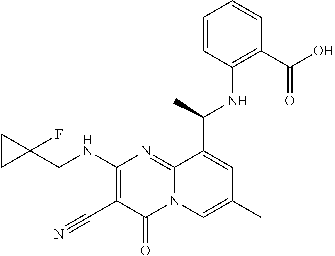 [SMILES: Cc1cc([C@@H](C)Nc2ccccc2C(=O)O)c2nc(NCC3(F)CC3)c(C#N)c(=O)n2c1] | (R)-2-((1-(3-cyano-2-(((1- fluorocyclopropyl)methyl)amino)- 7-methyl-4-oxo-4H-pyrido [1,2-a]pyrimidin-9- yl)ethyl)amino)benzoic acid |
| 11-156 | 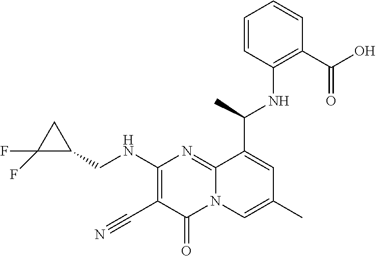 [SMILES: Cc1cc([C@@H](C)Nc2ccccc2C(=O)O)c2nc(NC[C@H]3CC3(F)F)c(C#N)c(=O)n2c1] | 2-(((R)-1-(3-cyano-2-((((R)-2,2- difluorocyclopropyl)methyl)amino)- 7-methyl-4-oxo- 4H-pyrido[1,2-a]pyrimidin-9- yl)ethyl)amino)benzoic acid |
| 11-157 | 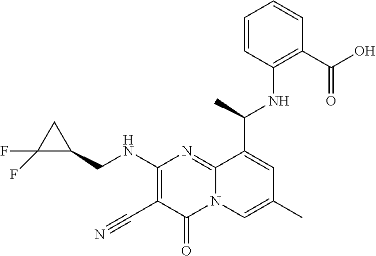 [SMILES: Cc1cc([C@@H](C)Nc2ccccc2C(=O)O)c2nc(NC[C@@H]3CC3(F)F)c(C#N)c(=O)n2c1] | 2-(((R)-1-(3-cyano-2-(((S)-2,2- difluorocyclopropyl)methyl)amino)- 7-methyl-4-oxo-4H-pyrido[1,2-a]pyrimidin-9- yl)ethyl)amino)benzoic acid |
| 11-158 | 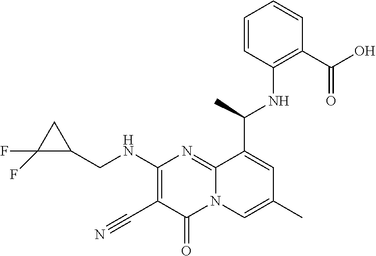 [SMILES: Cc1cc([C@@H](C)Nc2ccccc2C(=O)O)c2nc(NCC3CC3(F)F)c(C#N)c(=O)n2c1] | 2-(((R)-1-(3-cyano-2-(((2,2- difluorocyclopropyl)methyl)amino)- 7-methyl-4-oxo-4H- pyrido[1,2-a]pyrimidin-9- yl)ethyl)amino)benzoic acid |
| 11-159 | 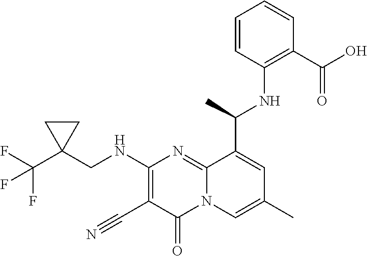 [SMILES: Cc1cc([C@@H](C)Nc2ccccc2C(=O)O)c2nc(NCC3(C(F)(F)F)CC3)c(C#N)c(=O)n2c1] | (R)-2-((1-(3-cyano-7-methyl-4- oxo-2-(((1- (trifluoromethyl)cyclopropyl)methyl) amino)-4H-pyrido[1,2-a]pyrimidin-9- yl)ethyl)amino)benzoic acid |
| 11-160 | 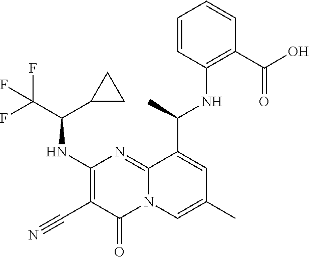 [SMILES: Cc1cc([C@@H](C)Nc2ccccc2C(=O)O)c2nc(N[C@H](C3CC3)C(F)(F)F)c(C#N)c(=O)n2c1] | 2-(((R)-1-(3-cyano-2-(((R)-1- cyclopropyl-2,2,2- trifluoroethyl)amino)-7-methyl-4- oxo-4H-pyrido[1,2-a]pyrimidin-9- yl)ethyl)amino)benzoic acid |
| 11-161 | 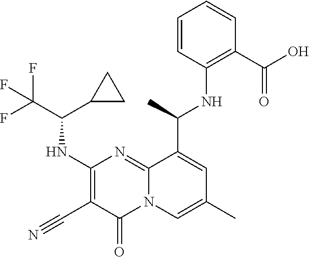 [SMILES: Cc1cc([C@@H](C)Nc2ccccc2C(=O)O)c2nc(N[C@@H](C3CC3)C(F)(F)F)c(C#N)c(=O)n2c1] | 2-(((R)-1-(3-cyano-2-(((S)-1- cyclopropyl-2,2,2- trifluoroethyl)amino)-7-methyl-4- oxo-4H-pyrido[1,2-a]pyrimidin-9- yl)ethyl)amino)benzoic acid |
| 11-162 | 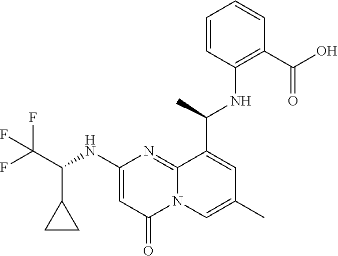 [SMILES: Cc1cc([C@@H](C)Nc2ccccc2C(=O)O)c2nc(N[C@H](C3CC3)C(F)(F)F)cc(=O)n2c1] | 2-(((R)-1-(2-(((R)-1-cyclopropyl- 2,2,2-trifluoroethyl)amino)-3,7- dimethyl-4-oxo-4H-pyrido[1,2-a]pyrimidin-9- yl)ethyl)amino)benzoic acid |
| 11-163 | 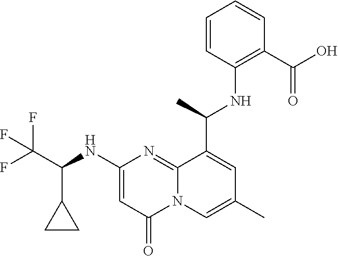 [SMILES: Cc1cc([C@@H](C)Nc2ccccc2C(=O)O)c2nc(N[C@@H](C3CC3)C(F)(F)F)cc(=O)n2c1] | 2-(((R)-1-(2-(((S)-1-cyclopropyl- 2,2,2-trifluoroethyl)amino)-3,7- dimethyl-4-oxo- 4H-pyrido[1,2-a]pyrimidin-9- yl)ethyl)amino)benzoic acid |
| 11-164 | 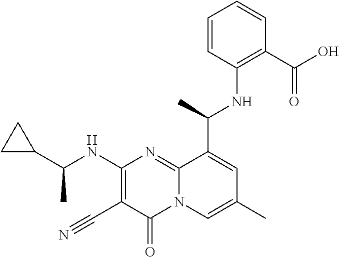 [SMILES: Cc1cc([C@@H](C)Nc2ccccc2C(=O)O)c2nc(N[C@@H](C)C3CC3)c(C#N)c(=O)n2c1] | 2-(((R)-1-(3-cyano-2-(((S)-1- cyclopropylethyl)amino)-7- methyl-4-oxo-4H-pyrido[1,2-a]pyrimidin-9- yl)ethyl)amino)benzoic acid |
| 11-165 | 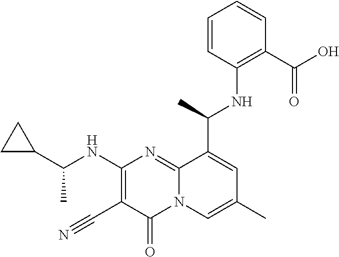 [SMILES: Cc1cc([C@@H](C)Nc2ccccc2C(=O)O)c2nc(N[C@H](C)C3CC3)c(C#N)c(=O)n2c1] | 2-(((R)-1-(3-cyano-2-(((R)-1- cyclopropylethyl)amino)-7- methyl-4-oxo- 4H-pyrido[1,2-a]pyrimidin-9- yl)ethyl)amino)benzoic acid |
| 11-166 | 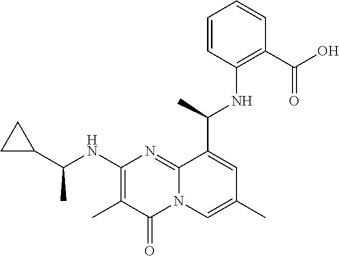 [SMILES: Cc1cc([C@@H](C)Nc2ccccc2C(=O)O)c2nc(N[C@@H](C)C3CC3)c(C)c(=O)n2c1] | 2-(((R)-1-(2-(((S)-1- cyclopropylethyl)amino)-3,7- dimethyl-4-oxo- 4H-pyrido[1,2-a]pyrimidin-9- yl)ethyl)amino)benzoic acid |
| 11-167 | 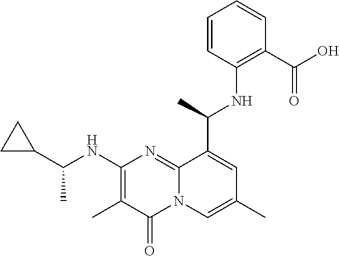 [SMILES: Cc1cc([C@@H](C)Nc2ccccc2C(=O)O)c2nc(N[C@H](C)C3CC3)c(C)c(=O)n2c1] | 2-(((R)-1-(2-(((R)-1- cyclopropylethyl)amino)-3,7- dimethyl-4-oxo- 4H-pyrido[1,2-a]pyrimidin-9- yl)ethyl)amino)benzoic acid |
| 11-168 | 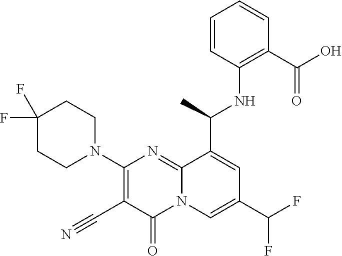 [SMILES: C[C@@H](Nc1ccccc1C(=O)O)c1cc(C(F)F)cn2c(=O)c(C#N)c(N3CCC(F)(F)CC3)nc12] | (R)-2-((1-(3-cyano-7- (difluoromethyl)-2-(4,4- difluoropiperidin-1-yl)-4-oxo- 4H-pyrido[1,2-a]pyrimidin-9- yl)ethyl)amino)benzoic acid |
| 11-169 | 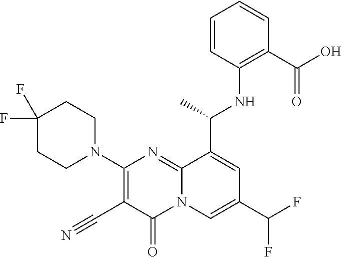 [SMILES: C[C@H](Nc1ccccc1C(=O)O)c1cc(C(F)F)cn2c(=O)c(C#N)c(N3CCC(F)(F)CC3)nc12] | (S)-2-((1-(3-cyano-7- (difluoromethyl)-2-(4,4- difluoropiperidin-1-yl)-4-oxo- 4H-pyrido[1,2-a]pyrimidin-9- yl)ethyl)amino)benzoic acid |
| 11-170 | 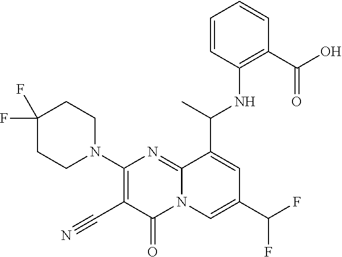 [SMILES: CC(Nc1ccccc1C(=O)O)c1cc(C(F)F)cn2c(=O)c(C#N)c(N3CCC(F)(F)CC3)nc12] | 2-((1-(3-cyano-7- (difluoromethyl)-2-(4,4- difluoropiperidin-1-yl)-4-oxo- 4H-pyrido[1,2-a]pyrimidin-9- yl)ethyl)amino)benzoic acid |
| 11-171 | 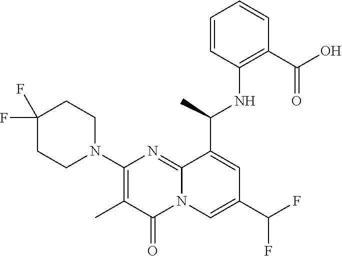 [SMILES: Cc1c(N2CCC(F)(F)CC2)nc2c([C@@H](C)Nc3ccccc3C(=O)O)cc(C(F)F)cn2c1=O] | (R)-2-((1-(7-(difluoromethyl)-2- (4,4-difluoropiperidin-1-yl)-3- methyl-4-oxo- 4H-pyrido[1,2-a]pyrimidin-9- yl)ethyl)amino)benzoic acid |
| 11-172 | 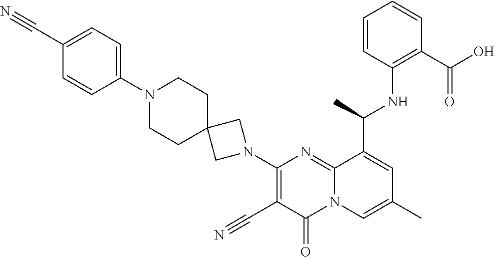 [SMILES: Cc1cc([C@@H](C)Nc2ccccc2C(=O)O)c2nc(N3CC4(CCN(c5ccc(C#N)cc5)CC4)C3)c(C#N)c(=O)n2c1] | (R)-2-((1-(3-cyano-2-(7-(4- cyanophenyl)-2,7- diazaspiro[3.5]nonan-2-yl)-7- methyl-4-oxo- 4H-pyrido[1,2-a]pyrimidin-9- yl)ethyl)amino)benzoic acid |
| 11-173 | 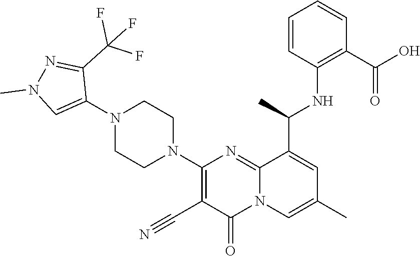 [SMILES: Cc1cc([C@@H](C)Nc2ccccc2C(=O)O)c2nc(N3CCN(c4cn(C)nc4C(F)(F)F)CC3)c(C#N)c(=O)n2c1] | (R)-2-((1-(3-cyano-7-methyl-2-(4- (1-methyl-3-(trifluoromethyl)-1H- pyrazol-4-yl)piperazin-1-yl)-4- oxo-4H-pyrido[1,2-a]pyrimidin-9- yl)ethyl)amino)benzoic acid |
| 11-174 | 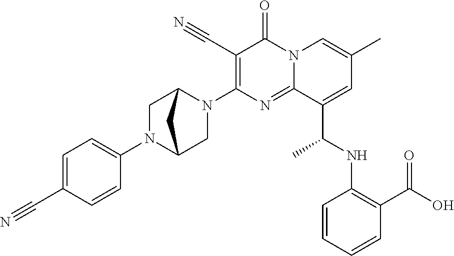 [SMILES: Cc1cc([C@@H](C)Nc2ccccc2C(=O)O)c2nc(N3C[C@H]4C[C@@H]3CN4c3ccc(C#N)cc3)c(C#N)c(=O)n2c1] | 2-(((R)-1-(3-cyano-2-((1R,4R)-5- (4-cyanophenyl)-2,5- diazabicyclo[2.2.1]heptan-2-yl)- 7-methyl-4-oxo- 4H-pyrido[1,2-a]pyrimidin-9- yl)ethyl)amino)benzoic acid |
| 11-175 | 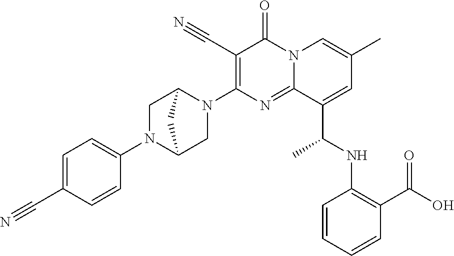 [SMILES: Cc1cc([C@@H](C)Nc2ccccc2C(=O)O)c2nc(N3C[C@@H]4C[C@H]3CN4c3ccc(C#N)cc3)c(C#N)c(=O)n2c1] | 2-(((R)-1-(3-cyano-2-((1S,4S)-5- (4-cyanophenyl)-2,5- diazabicyclo[2.2.1]heptan-2-yl)- 7-methyl-4-oxo- 4H-pyrido[1,2-a]pyrimidin-9- yl)ethyl)amino)benzoic acid |
| 11-176 | 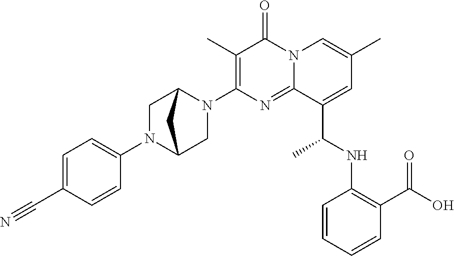 [SMILES: Cc1cc([C@@H](C)Nc2ccccc2C(=O)O)c2nc(N3C[C@H]4C[C@@H]3CN4c3ccc(C#N)cc3)c(C)c(=O)n2c1] | 2-(((R)-1-(2-((1R,4R)-5-(4- cyanophenyl)-2,5- diazabicyclo[2.2.1]heptan-2-yl)- 3,7-dimethyl-4-oxo- 4H-pyrido[1,2-a]pyrimidin-9- yl)ethyl)amino)benzoic acid |
| 11-177 | 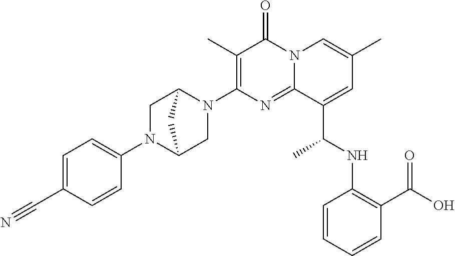 [SMILES: Cc1cc([C@@H](C)Nc2ccccc2C(=O)O)c2nc(N3C[C@@H]4C[C@H]3CN4c3ccc(C#N)cc3)c(C)c(=O)n2c1] | 2-(((R)-1-(2-((1S,4S)-5-(4- cyanophenyl)-2,5- diazabicyclo[2.2.1]heptan-2-yl)- 3,7-dimethyl-4-oxo- 4H-pyrido[1,2-a]pyrimidin-9- yl)ethyl)amino)benzoic acid |
| 11-178 | 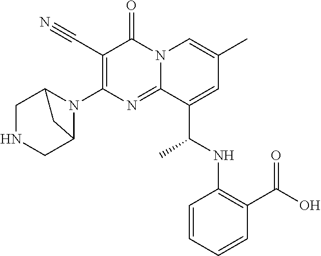 [SMILES: Cc1cc([C@@H](C)Nc2ccccc2C(=O)O)c2nc(N3C4CNCC3C4)c(C#N)c(=O)n2c1] | 2-(((1R)-1-(2-(3,6- diazabicyclo[3.1.1]heptan-6-yl)- 3-cyano-7-methyl-4-oxo- 4H-pyrido[1,2-a]pyrimidin-9- yl)ethyl)amino)benzoic acid |
| 11-179 | 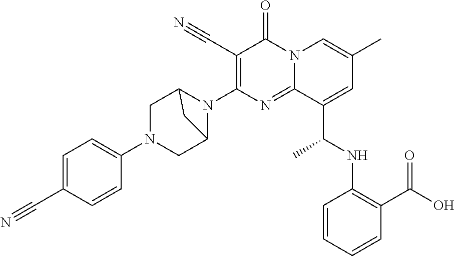 [SMILES: Cc1cc([C@@H](C)Nc2ccccc2C(=O)O)c2nc(N3C4CC3CN(c3ccc(C#N)cc3)C4)c(C#N)c(=O)n2c1] | 2-(((1R)-1-(3-cyano-2-(3-(4- cyanophenyl)-3,6- diazabicyclo[3.1.1]heptan-6-yl)- 7-methyl-4-oxo- 4H-pyrido[1,2-a]pyrimidin-9- yl)ethyl)amino)benzoic acid |
| 11-180 | 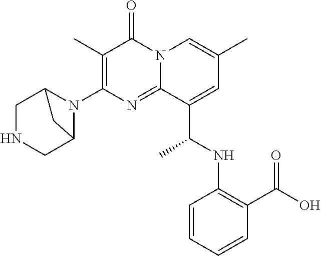 [SMILES: Cc1cc([C@@H](C)Nc2ccccc2C(=O)O)c2nc(N3C4CNCC3C4)c(C)c(=O)n2c1] | 2-(((1R)-1-(2-(3,6- diazabicyclo[3.1.1]heptan-6-yl)- 3,7-dimethyl-4-oxo- 4H-pyrido[1,2-a]pyrimidin-9- yl)ethyl)amino)benzoic acid |
| 11-181 | 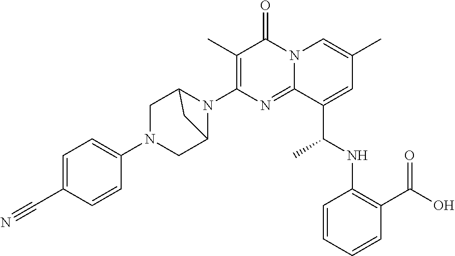 [SMILES: Cc1cc([C@@H](C)Nc2ccccc2C(=O)O)c2nc(N3C4CC3CN(c3ccc(C#N)cc3)C4)c(C)c(=O)n2c1] | 2-(((1R)-1-(2-(3-(4-cyanophenyl)- 3,6-diazabicyclo[3.1.1]heptan-6- yl)-3,7-dimethyl-4-oxo- 4H-pyrido[1,2-a]pyrimidin-9- yl)ethyl)amino)benzoic acid |
| 11-182 | 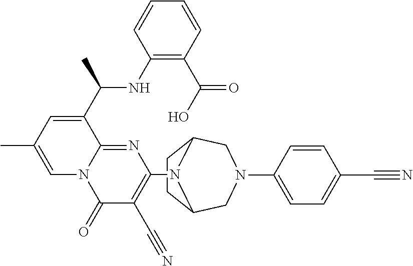 [SMILES: Cc1cc([C@@H](C)Nc2ccccc2C(=O)O)c2nc(N3C4CCC3CN(c3ccc(C#N)cc3)C4)c(C#N)c(=O)n2c1] | 2-(((1R)-1-(3-cyano-2-(3-(4- cyanophenyl)-3,8- diazabicyclo[3.2.1]octan-8-yl)-7- methyl-4-oxo- 4H-pyrido[1,2-a]pyrimidin-9- yl)ethyl)amino)benzoic acid |
| 11-183 | 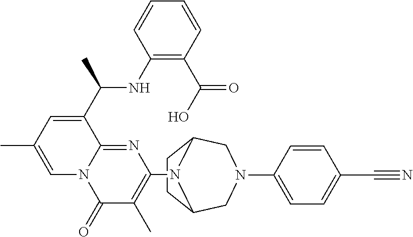 [SMILES: Cc1cc([C@@H](C)Nc2ccccc2C(=O)O)c2nc(N3C4CCC3CN(c3ccc(C#N)cc3)C4)c(C)c(=O)n2c1] | 2-(((1R)-1-(2-(3-(4-cyanophenyl)- 3,8-diazabicyclo[3.2.1]octan-8- yl)-3,7-dimethyl-4-oxo- 4H-pyrido[1,2-a]pyrimidin-9- yl)ethyl)amino)benzoic acid |
| 11-184 | 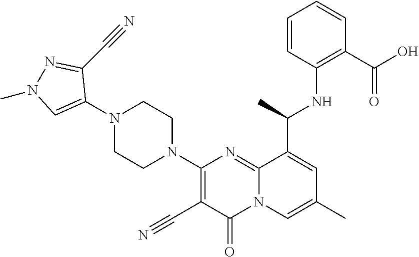 [SMILES: Cc1cc([C@@H](C)Nc2ccccc2C(=O)O)c2nc(N3CCN(c4cn(C)nc4C#N)CC3)c(C#N)c(=O)n2c1] | (R)-2-((1-(3-cyano-2-(4-(3- cyano-1-methyl-1H-pyrazol-4- yl)piperazin-1-yl)-7-methyl-4- oxo-4H-pyrido[1,2-a]pyrimidin-9- yl)ethyl)amino)benzoic acid |
| 11-185 | 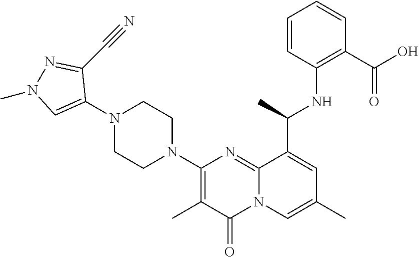 [SMILES: Cc1cc([C@@H](C)Nc2ccccc2C(=O)O)c2nc(N3CCN(c4cn(C)nc4C#N)CC3)c(C)c(=O)n2c1] | (R)-2-((1-(2-(4-(3-cyano-1- methyl-1H-pyrazol-4- yl)piperazin-1-yl)-3,7-dimethyl-4- oxo-4H-pyrido[1,2-a]pyrimidin-9- yl)ethyl)amino)benzoic acid |
| 11-186 | 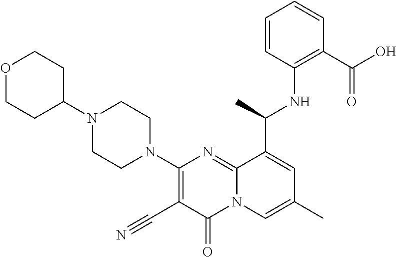 [SMILES: Cc1cc([C@@H](C)Nc2ccccc2C(=O)O)c2nc(N3CCN(C4CCOCC4)CC3)c(C#N)c(=O)n2c1] | (R)-2-((1-(3-cyano-7-methyl-4- oxo-2-(4-(tetrahydro-2H-pyran-4- yl)piperazin-1-yl)- 4H-pyrido[1,2-a]pyrimidin-9- yl)ethyl)amino)benzoic acid |
| 11-187 | 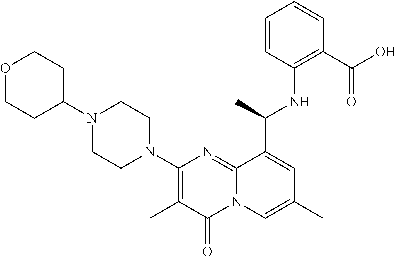 [SMILES: Cc1cc([C@@H](C)Nc2ccccc2C(=O)O)c2nc(N3CCN(C4CCOCC4)CC3)c(C)c(=O)n2c1] | (R)-2-((1-(3,7-dimethyl-4-oxo-2- (4-(tetrahydro-2H-pyran-4- yl)piperazin-1-yl)- 4H-pyrido[1,2-a]pyrimidin-9- yl)ethyl)amino)benzoic acid |
| 11-188 | 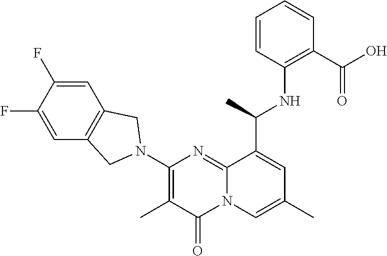 [SMILES: Cc1cc([C@@H](C)Nc2ccccc2C(=O)O)c2nc(N3Cc4cc(F)c(F)cc4C3)c(C)c(=O)n2c1] | (R)-2-((1-(2-(5,6- difluoroisoindolin-2-yl)-3,7- dimethyl-4-oxo- 4H-pyrido[1,2-a]pyrimidin-9- yl)ethyl)amino)benzoic acid |
| 11-189 | 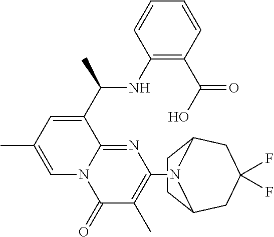 [SMILES: Cc1cc([C@@H](C)Nc2ccccc2C(=O)O)c2nc(N3C4CCC3CC(F)(F)C4)c(C)c(=O)n2c1] | 2-(((1R)-1-(2-(3,3-difluoro-8- azabicyclo[3.2.1]octan-8-yl)-3,7- dimethyl-4-oxo- 4H-pyrido[1,2-a]pyrimidin-9- yl)ethyl)amino)benzoic acid |
| 11-190 | 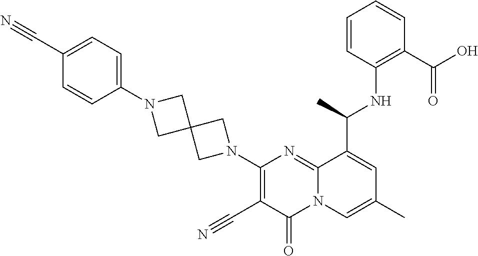 [SMILES: Cc1cc([C@@H](C)Nc2ccccc2C(=O)O)c2nc(N3CC4(CN(c5ccc(C#N)cc5)C4)C3)c(C#N)c(=O)n2c1] | (R)-2-((1-(3-cyano-2-(6-(4- cyanophenyl)-2,6- diazaspiro[3.3]heptan-2-yl)-7- methyl-4-oxo- 4H-pyrido[1,2-a]pyrimidin-9- yl)ethyl)amino)benzoic acid |
| 11-191 | 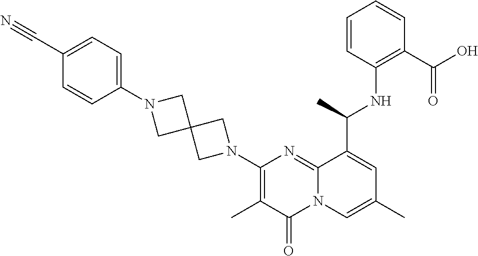 [SMILES: Cc1cc([C@@H](C)Nc2ccccc2C(=O)O)c2nc(N3CC4(CN(c5ccc(C#N)cc5)C4)C3)c(C)c(=O)n2c1] | (R)-2-((1-(2-(6-(4-cyanophenyl)- 2,6-diazaspiro[3.3]heptan-2-yl)- 3,7-dimethyl-4-oxo- 4H-pyrido[1,2-a]pyrimidin-9- yl)ethyl)amino)benzoic acid |
| 11-192 | 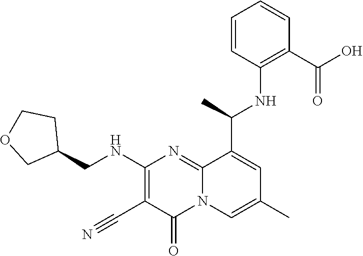 [SMILES: Cc1cc([C@@H](C)Nc2ccccc2C(=O)O)c2nc(NC[C@@H]3CCOC3)c(C#N)c(=O)n2c1] | 2-(((R)-1-(3-cyano-7-methyl-4- oxo-2-((((S)-tetrahydrofuran-3- yl)methyl)amino)- 4H-pyrido[1,2-a]pyrimidin-9- yl)ethyl)amino)benzoic acid |
| 11-193 | 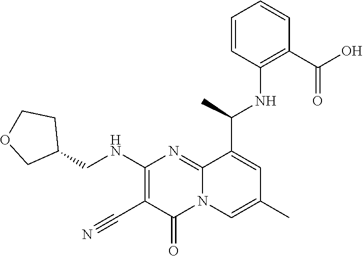 [SMILES: Cc1cc([C@@H](C)Nc2ccccc2C(=O)O)c2nc(NC[C@H]3CCOC3)c(C#N)c(=O)n2c1] | 2-(((R)-1-(3-cyano-7-methyl-4- oxo-2-((((R)-tetrahydrofuran-3- yl)methyl)amino)- 4H-pyrido[1,2-a]pyrimidin-9- yl)ethyl)amino)benzoic acid |
| 11-194 | 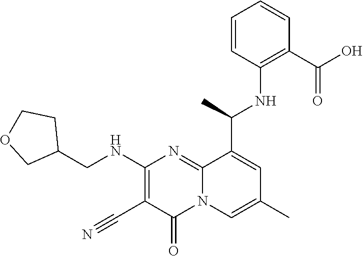 [SMILES: Cc1cc([C@@H](C)Nc2ccccc2C(=O)O)c2nc(NCC3CCOC3)c(C#N)c(=O)n2c1] | 2-(((1R)-1-(3-cyano-7-methyl-4- oxo-2-(((tetrahydrofuran-3- yl)methyl)amino)- 4H-pyrido[1,2-a]pyrimidin-9- yl)ethyl)amino)benzoic acid |
| 11-195 | 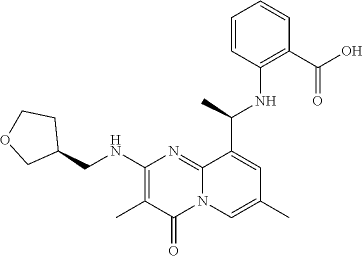 [SMILES: Cc1cc([C@@H](C)Nc2ccccc2C(=O)O)c2nc(NC[C@@H]3CCOC3)c(C)c(=O)n2c1] | 2-(((R)-1-(3,7-dimethyl-4-oxo-2- ((((S)-tetrahydrofuran-3- yl)methyl)amino)-4H-pyrido[1,2- a]pyrimidin-9- yl)ethyl)amino)benzoic acid |
| 11-196 | 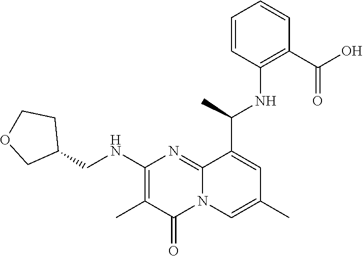 [SMILES: Cc1cc([C@@H](C)Nc2ccccc2C(=O)O)c2nc(NC[C@H]3CCOC3)c(C)c(=O)n2c1] | 2-(((R)-1-(3,7-dimethyl-4-oxo-2- ((((R)-tetrahydrofuran-3- yl)methyl)amino)- 4H-pyrido[1,2-a]pyrimidin-9- yl)ethyl)amino)benzoic acid |
| 11-197 | 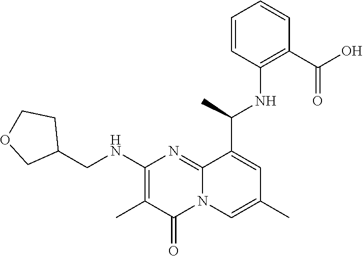 [SMILES: Cc1cc([C@@H](C)Nc2ccccc2C(=O)O)c2nc(NCC3CCOC3)c(C)c(=O)n2c1] | 2-(((1R)-1-(3,7-dimethyl-4-oxo-2- (((tetrahydrofuran-3- yl)methyl)amino)- 4H-pyrido[1,2-a]pyrimidin-9- yl)ethyl)amino)benzoic acid |
| 11-198 | 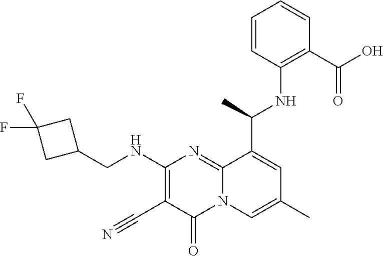 [SMILES: Cc1cc([C@@H](C)Nc2ccccc2C(=O)O)c2nc(NCC3CC(F)(F)C3)c(C#N)c(=O)n2c1] | (R)-2-((1-(3-cyano-2-(((3,3- difluorocyclobutyl)methyl)amino)- 7-methyl-4-oxo-4H-pyrido [1,2-a]pyrimidin-9- yl)ethyl)amino)benzoic acid |
| 11-199 | 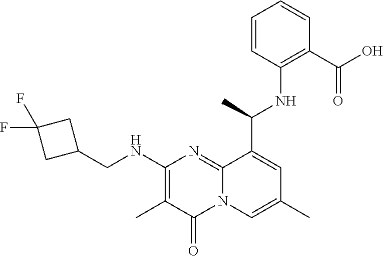 [SMILES: Cc1cc([C@@H](C)Nc2ccccc2C(=O)O)c2nc(NCC3CC(F)(F)C3)c(C)c(=O)n2c1] | (R)-2-((1-(2-(((3,3- difluorocyclobutyl)methyl)amino)- 3,7-dimethyl-4-oxo- 4H-pyrido[1,2-a]pyrimidin-9- yl)ethyl)amino)benzoic acid |
| 11-200 | 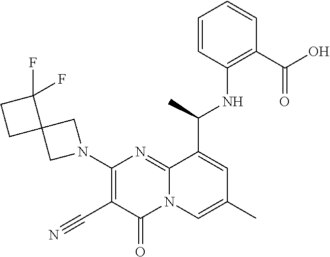 [SMILES: Cc1cc([C@@H](C)Nc2ccccc2C(=O)O)c2nc(N3CC4(CCC4(F)F)C3)c(C#N)c(=O)n2c1] | (R)-2-((1-(3-cyano-2-(5,5- difluoro-2-azaspiro[3.3]heptan-2- yl)-7-methyl-4-oxo- 4H-pyrido[1,2-a]pyrimidin-9- yl)ethyl)amino)benzoic acid |
| 11-201 | 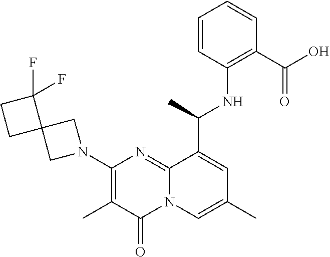 [SMILES: Cc1cc([C@@H](C)Nc2ccccc2C(=O)O)c2nc(N3CC4(CCC4(F)F)C3)c(C)c(=O)n2c1] | (R)-2-((1-(2-(5,5-difluoro-2- azaspiro[3.3]heptan-2-yl)-3,7- dimethyl-4-oxo- 4H-pyrido[1,2-a]pyrimidin-9- yl)ethyl)amino)benzoic acid |
| 11-202 | 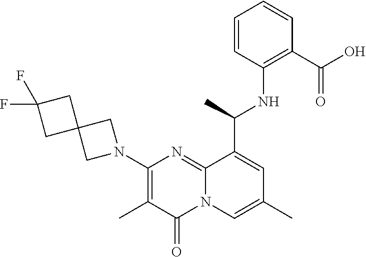 [SMILES: Cc1cc([C@@H](C)Nc2ccccc2C(=O)O)c2nc(N3CC4(C3)CC(F)(F)C4)c(C)c(=O)n2c1] | (R)-2-((1-(2-(6,6-difluoro-2- azaspiro[3.3]heptan-2-yl)-3,7- dimethyl-4-oxo- 4H-pyrido[1,2-a]pyrimidin-9- yl)ethyl)amino)benzoic acid |
| 11-203 | 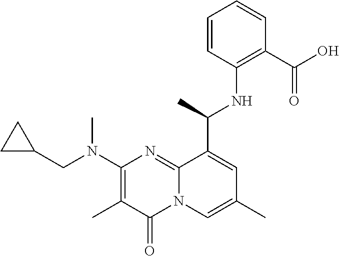 [SMILES: Cc1cc([C@@H](C)Nc2ccccc2C(=O)O)c2nc(N(C)CC3CC3)c(C)c(=O)n2c1] | (R)-2-((1-(2- ((cyclopropylmethyl)(methyl)amino)- 3,7-dimethyl-4-oxo- 4H-pyrido[1,2-a]pyrimidin-9- yl)ethyl)amino)benzoic acid |
| 11-204 | 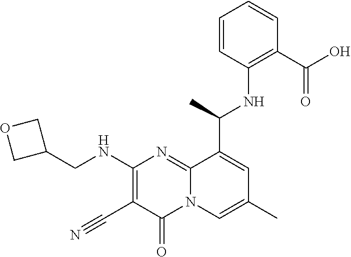 [SMILES: Cc1cc([C@@H](C)Nc2ccccc2C(=O)O)c2nc(NCC3COC3)c(C#N)c(=O)n2c1] | (R)-2-((1-(3-cyano-7-methyl-2- ((oxetan-3-ylmethyl)amino)-4- oxo-4H-pyrido[1,2-a]pyrimidin-9- yl)ethyl)amino)benzoic acid |
| 11-205 | 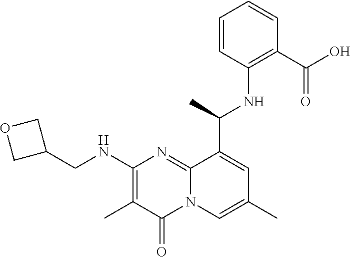 [SMILES: Cc1cc([C@@H](C)Nc2ccccc2C(=O)O)c2nc(NCC3COC3)c(C)c(=O)n2c1] | (R)-2-((1-(3,7-dimethyl-2- ((oxetan-3-ylmethyl)amino)-4- oxo-4H-pyrido[1,2-a]pyrimidin-9- yl)ethyl)amino)benzoic acid |
| 11-206 | 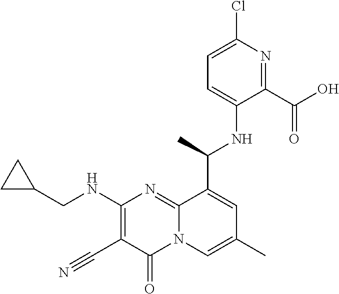 [SMILES: Cc1cc([C@@H](C)Nc2ccc(Cl)nc2C(=O)O)c2nc(NCC3CC3)c(C#N)c(=O)n2c1] | (R)-6-chloro-3-((1-(3-cyano-2- ((cyclopropylmethyl)amino)-7- methyl-4-oxo-4H-pyrido[1,2-a]pyrimidin-9- yl)ethyl)amino)picolinic acid |
| 11-207 | 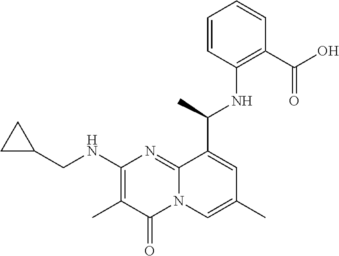 [SMILES: Cc1cc([C@@H](C)Nc2ccccc2C(=O)O)c2nc(NCC3CC3)c(C)c(=O)n2c1] | (R)-2-((1-(2- ((cyclopropylmethyl)amino)-3,7- dimethyl-4-oxo- 4H-pyrido[1,2-a]pyrimidin-9- yl)ethyl)amino)benzoic acid |
| 11-208 | 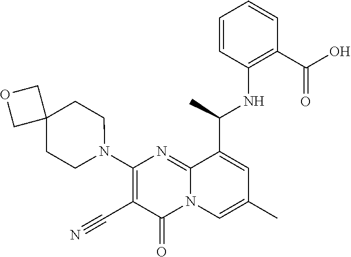 [SMILES: Cc1cc([C@@H](C)Nc2ccccc2C(=O)O)c2nc(N3CCC4(CC3)COC4)c(C#N)c(=O)n2c1] | (R)-2-((1-(3-cyano-7-methyl-4- oxo-2-(2-oxa-7- azaspiro[3.5]nonan-7-yl)- 4H-pyrido[1,2-a]pyrimidin-9- yl)ethyl)amino)benzoic acid |
| 11-209 | 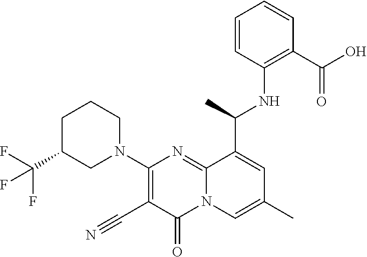 [SMILES: Cc1cc([C@@H](C)Nc2ccccc2C(=O)O)c2nc(N3CCC[C@@H](C(F)(F)F)C3)c(C#N)c(=O)n2c1] | 2-(((R)-1-(3-cyano-7-methyl-4- oxo-2-((R)-3- (trifluoromethyl)piperidin-1-yl)- 4H-pyrido[1,2-a]pyrimidin-9- yl)ethyl)amino)benzoic acid |
| 11-210 | 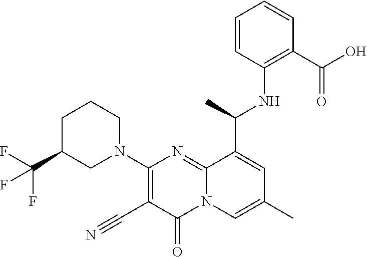 [SMILES: Cc1cc([C@@H](C)Nc2ccccc2C(=O)O)c2nc(N3CCC[C@H](C(F)(F)F)C3)c(C#N)c(=O)n2c1] | 2-(((R)-1-(3-cyano-7-methyl-4- oxo-2-((S)-3- (trifluoromethyl)piperidin-1-yl)- 4H-pyrido[1,2-a]pyrimidin-9- yl)ethyl)amino)benzoic acid |
| 11-211 | 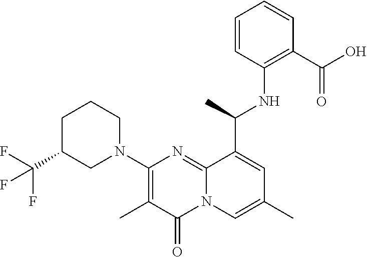 [SMILES: Cc1cc([C@@H](C)Nc2ccccc2C(=O)O)c2nc(N3CCC[C@@H](C(F)(F)F)C3)c(C)c(=O)n2c1] | 2-(((R)-1-(3,7-dimethyl-4-oxo-2- ((R)-3-(trifluoromethyl)piperidin- 1-yl)-4H-pyrido[1,2-a]pyrimidin-9- yl)ethyl)amino)benzoic acid |
| 11-212 | 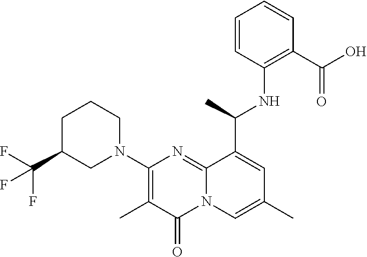 [SMILES: Cc1cc([C@@H](C)Nc2ccccc2C(=O)O)c2nc(N3CCC[C@H](C(F)(F)F)C3)c(C)c(=O)n2c1] | 2-(((R)-1-(3,7-dimethyl-4-oxo-2- ((S)-3-(trifluoromethyl)piperidin- 1-yl)-4H-pyrido[1,2-a]pyrimidin-9- yl)ethyl)amino)benzoic acid |
| 11-213 | 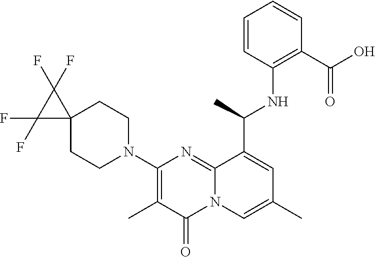 [SMILES: Cc1cc([C@@H](C)Nc2ccccc2C(=O)O)c2nc(N3CCC4(CC3)C(F)(F)C4(F)F)c(C)c(=O)n2c1] | (R)-2-((1-(3,7-dimethyl-4-oxo-2- (1,1,2,2-tetrafluoro-6- azaspiro[2.5]octan-6-yl)- 4H-pyrido[1,2-a]pyrimidin-9- yl)ethyl)amino)benzoic acid |
| 11-214 | 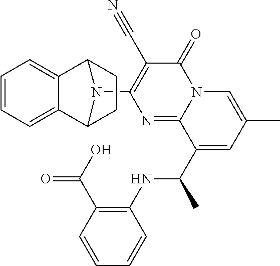 [SMILES: Cc1cc([C@@H](C)Nc2ccccc2C(=O)O)c2nc(N3C4CCC3c3ccccc34)c(C#N)c(=O)n2c1] | 2-(((1R)-1-(3-cyano-7-methyl-4- oxo-2-(1,2,3,4-tetrahydro-1,4- epiminonaphthalen-9-yl)- 4H-pyrido[1,2-a]pyrimidin-9- yl)ethyl)amino)benzoic acid |
| 11-215 | 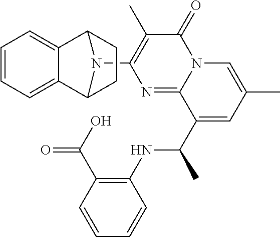 [SMILES: Cc1cc([C@@H](C)Nc2ccccc2C(=O)O)c2nc(N3C4CCC3c3ccccc34)c(C)c(=O)n2c1] | 2-(((1R)-1-(3,7-dimethyl-4-oxo-2- (1,2,3,4-tetrahydro-1,4- epiminonaphthalen-9-yl)-4H- pyrido[1,2-a]pyrimidin-9- yl)ethyl)amino)benzoic acid |
| 11-216 | 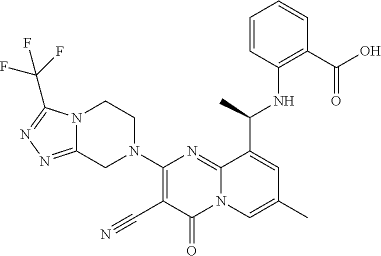 [SMILES: Cc1cc([C@@H](C)Nc2ccccc2C(=O)O)c2nc(N3CCn4c(nnc4C(F)(F)F)C3)c(C#N)c(=O)n2c1] | (R)-2-((1-(3-cyano-7-methyl-4- oxo-2-(3-(trifluoromethyl)-5,6- dihydro-[1,2,4]triazolo[4,3- a]pyrazin-7(8H)-yl)- 4H-pyrido[1,2-a]pyrimidin-9- yl)ethyl)amino)benzoic acid |
| 11-217 | 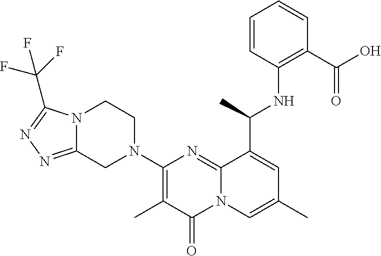 [SMILES: Cc1cc([C@@H](C)Nc2ccccc2C(=O)O)c2nc(N3CCn4c(nnc4C(F)(F)F)C3)c(C)c(=O)n2c1] | (R)-2-((1-(3,7-dimethyl-4-oxo-2- (3-(trifluoromethyl)-5,6-dihydro- [1,2,4]triazolo[4,3-a]pyrazin- 7(8H)-yl)-4H-pyrido[1,2- a]pyrimidin-9- yl)ethyl)amino)benzoic acid |
| 11-218 | 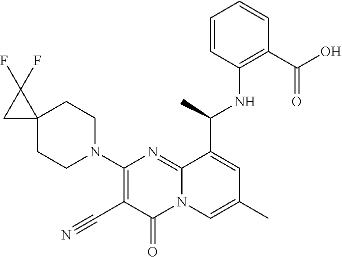 [SMILES: Cc1cc([C@@H](C)Nc2ccccc2C(=O)O)c2nc(N3CCC4(CC3)CC4(F)F)c(C#N)c(=O)n2c1] | (R)-2-((1-(3-cyano-2-(1,1- difluoro-6-azaspiro[2.5]octan-6- yl)-7-methyl-4-oxo- 4H-pyrido[1,2-a]pyrimidin-9- yl)ethyl)amino)benzoic acid |
| 11-219 | 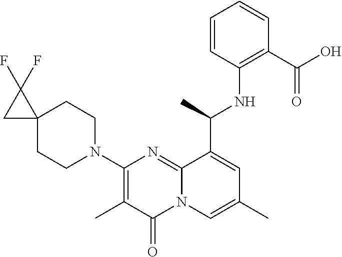 [SMILES: Cc1cc([C@@H](C)Nc2ccccc2C(=O)O)c2nc(N3CCC4(CC3)CC4(F)F)c(C)c(=O)n2c1] | (R)-2-((1-(2-(1,1-difluoro-6- azaspiro[2.5]octan-6-yl)-3,7- dimethyl-4-oxo- 4H-pyrido[1,2-a]pyrimidin-9- yl)ethyl)amino)benzoic acid |
| 11-220 | 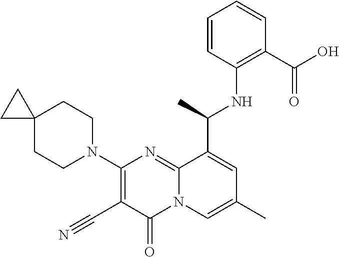 [SMILES: Cc1cc([C@@H](C)Nc2ccccc2C(=O)O)c2nc(N3CCC4(CC3)CC4)c(C#N)c(=O)n2c1] | (R)-2-((1-(3-cyano-7-methyl-4- oxo-2-(6-azaspiro[2.5]octan-6- yl)-4H-pyrido[1,2-a]pyrimidin-9- yl)ethyl)amino)benzoic acid |
| 11-221 | 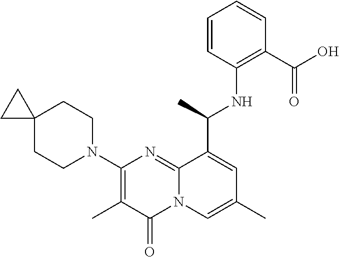 [SMILES: Cc1cc([C@@H](C)Nc2ccccc2C(=O)O)c2nc(N3CCC4(CC3)CC4)c(C)c(=O)n2c1] | (R)-2-((1-(3,7-dimethyl-4-oxo-2- (6-azaspiro[2.5]octan-6-yl)- 4H-pyrido[1,2-a]pyrimidin-9- yl)ethyl)amino)benzoic acid |
| 11-222 | 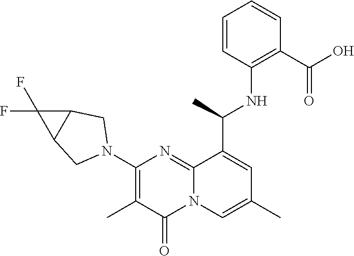 [SMILES: Cc1cc([C@@H](C)Nc2ccccc2C(=O)O)c2nc(N3CC4C(C3)C4(F)F)c(C)c(=O)n2c1] | 2-(((R)-1-(2-(6,6-difluoro-3- azabicyclo[3.1.0]hexan-3-yl)-3,7- dimethyl-4-oxo- 4H-pyrido[1,2-a]pyrimidin-9- yl)ethyl)amino)benzoic acid |
| 11-223 | 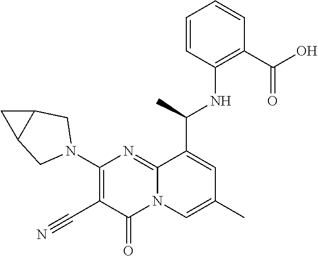 [SMILES: Cc1cc([C@@H](C)Nc2ccccc2C(=O)O)c2nc(N3CC4CC4C3)c(C#N)c(=O)n2c1] | 2-(((R)-1-(2-(3- azabicyclo[3.1.0]hexan-3-yl)-3- cyano-7-methyl-4-oxo- 4H-pyrido[1,2-a]pyrimidin-9- yl)ethyl)amino)benzoic acid |
| 11-224 | 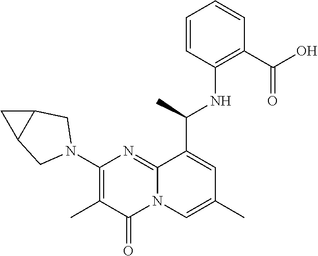 [SMILES: Cc1cc([C@@H](C)Nc2ccccc2C(=O)O)c2nc(N3CC4CC4C3)c(C)c(=O)n2c1] | 2-(((1R)-1-(2-(3- azabicyclo[3.1.0]hexan-3-yl)-3,7- dimethyl-4-oxo- 4H-pyrido[1,2-a]pyrimidin-9- yl)ethyl)amino)benzoic acid |
| 11-225 | 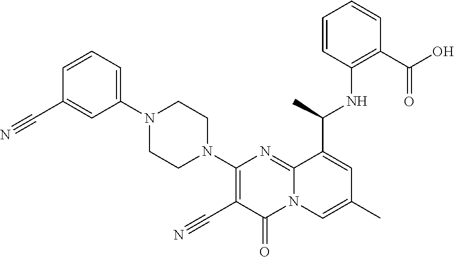 [SMILES: Cc1cc([C@@H](C)Nc2ccccc2C(=O)O)c2nc(N3CCN(c4cccc(C#N)c4)CC3)c(C#N)c(=O)n2c1] | (R)-2-((1-(3-cyano-2-(4-(3- cyanophenyl)piperazin-1-yl)-7- methyl-4-oxo- 4H-pyrido[1,2-a]pyrimidin-9- yl)ethyl)amino)benzoic acid |
| 11-226 | 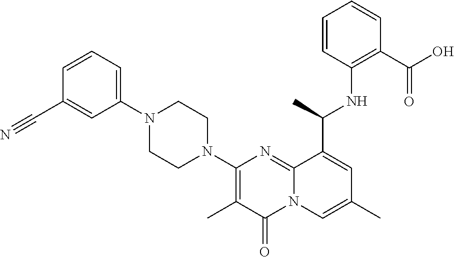 [SMILES: Cc1cc([C@@H](C)Nc2ccccc2C(=O)O)c2nc(N3CCN(c4cccc(C#N)c4)CC3)c(C)c(=O)n2c1] | (R)-2-((1-(2-(4-(3- cyanophenyl)piperazin-1-yl)-3,7- dimethyl-4-oxo- 4H-pyrido[1,2-a]pyrimidin-9- yl)ethyl)amino)benzoic acid |
| 11-227 | 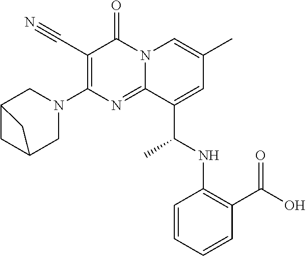 [SMILES: Cc1cc([C@@H](C)Nc2ccccc2C(=O)O)c2nc(N3CC4CC(C4)C3)c(C#N)c(=O)n2c1] | (R)-2-((1-(2-(3- azabicyclo[3.1.1]heptan-3-yl)-3- cyano-7-methyl-4-oxo- 4H-pyrido[1,2-a]pyrimidin-9- yl)ethyl)amino)benzoic acid |
| 11-228 | 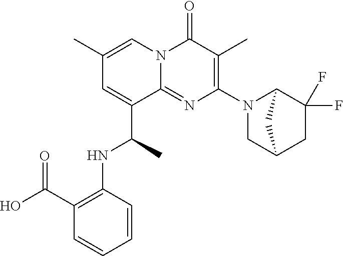 [SMILES: Cc1cc([C@@H](C)Nc2ccccc2C(=O)O)c2nc(N3C[C@H]4C[C@@H]3C(F)(F)C4)c(C)c(=O)n2c1] | 2-(((R)-1-(2-((1R,4S)-6,6- difluoro-2- azabicyclo[2.2.1]heptan-2-yl)- 3,7-dimethyl-4-oxo- 4H-pyrido[1,2-a]pyrimidin-9- yl)ethyl)amino)benzoic acid |
| 11-229 | 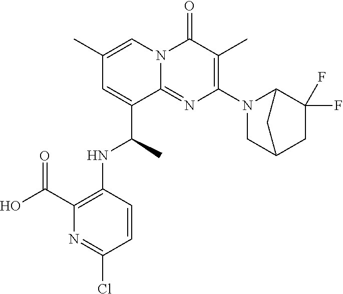 [SMILES: Cc1cc([C@@H](C)Nc2ccc(Cl)nc2C(=O)O)c2nc(N3CC4CC3C(F)(F)C4)c(C)c(=O)n2c1] | 6-chloro-3-(((1R)-1-(2-(6,6- difluoro-2- azabicyclo[2.2.1]heptan-2-yl)- 3,7-dimethyl-4-oxo- 4H-pyrido[1,2-a]pyrimidin-9- yl)ethyl)amino)picolinic acid |
| 11-230 | 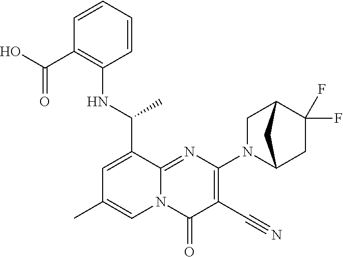 [SMILES: Cc1cc([C@@H](C)Nc2ccccc2C(=O)O)c2nc(N3C[C@H]4C[C@@H]3CC4(F)F)c(C#N)c(=O)n2c1] | 2-(((R)-1-(3-cyano-2-((1R,4R)- 5,5-difluoro-2- azabicyclo[2.2.1]heptan-2-yl)-7- methyl-4-oxo- 4H-pyrido[1,2-a]pyrimidin-9- yl)ethyl)amino)benzoic acid |
| 11-231 | 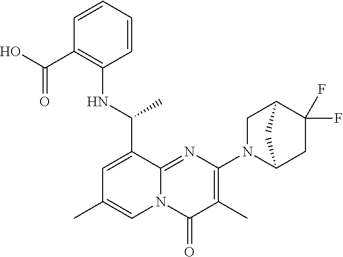 [SMILES: Cc1cc([C@@H](C)Nc2ccccc2C(=O)O)c2nc(N3C[C@@H]4C[C@H]3CC4(F)F)c(C)c(=O)n2c1] | 2-(((R)-1-(2-((1S,4S)-5,5- difluoro-2- azabicyclo[2.2.1]heptan-2-yl)- 3,7-dimethyl-4-oxo- 4H-pyrido[1,2-a]pyrimidin-9- yl)ethyl)amino)benzoic acid |
| 11-232 | 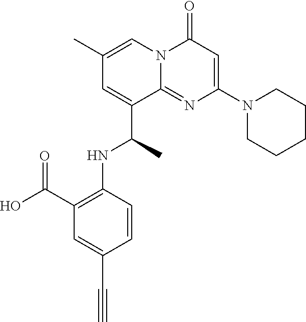 [SMILES: C#Cc1ccc(N[C@H](C)c2cc(C)cn3c(=O)cc(N4CCCCC4)nc23)c(C(=O)O)c1] | (R)-5-ethynyl-2-((1-(7-methyl-4- oxo-2-(piperidin-1-yl)- 4H-pyrido[1,2-a]pyrimidin-9- yl)ethyl)amino)benzoic acid |
| 11-233 | 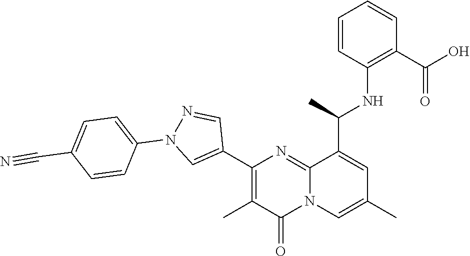 [SMILES: Cc1cc([C@@H](C)Nc2ccccc2C(=O)O)c2nc(-c3cnn(-c4ccc(C#N)cc4)c3)c(C)c(=O)n2c1] | (R)-2-((1-(2-(1-(4-cyanophenyl)- 1H-pyrazol-4-yl)-3,7-dimethyl-4- oxo-4H-pyrido[1,2-a]pyrimidin-9- yl)ethyl)amino)benzoic acid |
| 11-234 | 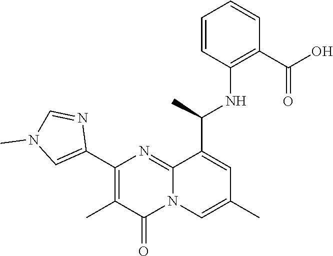 [SMILES: Cc1cc([C@@H](C)Nc2ccccc2C(=O)O)c2nc(-c3cn(C)cn3)c(C)c(=O)n2c1] | (R)-2-((1-(3,7-dimethyl-2-(1- methyl-1H-imidazol-4-yl)-4-oxo- 4H-pyrido[1,2-a]pyrimidin-9- yl)ethyl)amino)benzoic acid |
| 11-235 | 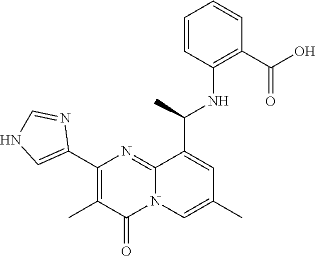 [SMILES: Cc1cc([C@@H](C)Nc2ccccc2C(=O)O)c2nc(-c3c[nH]cn3)c(C)c(=O)n2c1] | (R)-2-((1-(2-(1H-imidazol-4-yl)- 3,7-dimethyl-4-oxo- 4H-pyrido[1,2-a]pyrimidin-9- yl)ethyl)amino)benzoic acid |
| 11-236 | 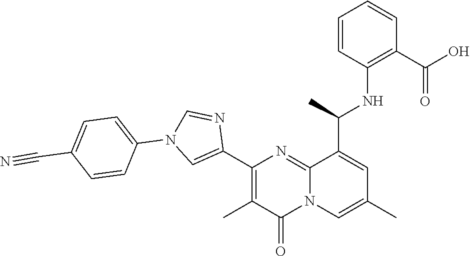 [SMILES: Cc1cc([C@@H](C)Nc2ccccc2C(=O)O)c2nc(-c3cn(-c4ccc(C#N)cc4)cn3)c(C)c(=O)n2c1] | (R)-2-((1-(2-(1-(4-cyanophenyl)- 1H-imidazol-4-yl)-3,7-dimethyl-4- oxo-4H-pyrido[1,2-a]pyrimidin-9- yl)ethyl)amino)benzoic acid |
| 11-237 | 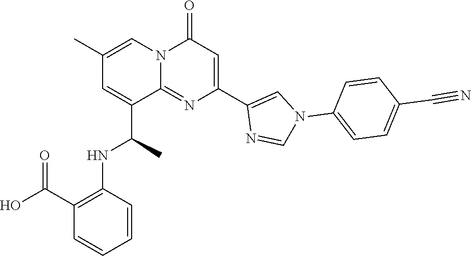 [SMILES: Cc1cc([C@@H](C)Nc2ccccc2C(=O)O)c2nc(-c3cn(-c4ccc(C#N)cc4)cn3)cc(=O)n2c1] | (R)-2-((1-(2-(1-(4-cyanophenyl)- 1H-imidazol-4-yl)-7-methyl-4- oxo-4H-pyrido[1,2-a]pyrimidin-9- yl)ethyl)amino)benzoic acid |
| 11-238 | 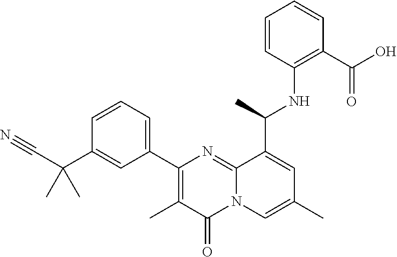 [SMILES: Cc1cc([C@@H](C)Nc2ccccc2C(=O)O)c2nc(-c3cccc(C(C)(C)C#N)c3)c(C)c(=O)n2c1] | (R)-2-((1-(2-(3-(2-cyanopropan- 2-yl)phenyl)-3,7-dimethyl-4-oxo- 4H-pyrido[1,2-a]pyrimidin-9- yl)ethyl)amino)benzoic acid |
| 11-239 | 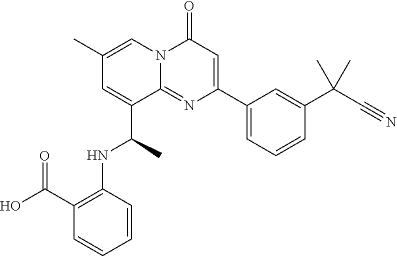 [SMILES: Cc1cc([C@@H](C)Nc2ccccc2C(=O)O)c2nc(-c3cccc(C(C)(C)C#N)c3)cc(=O)n2c1] | (R)-2-((1-(2-(3-(2-cyanopropan- 2-yl)phenyl)-7-methyl-4-oxo- 4H-pyrido[1,2-a]pyrimidin-9- yl)ethyl)amino)benzoic acid |
| 11-240 | 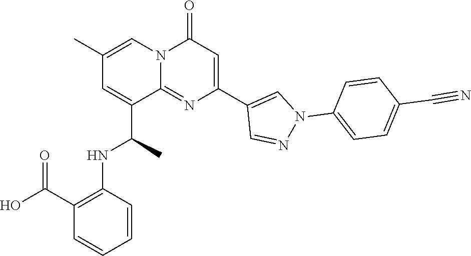 [SMILES: Cc1cc([C@@H](C)Nc2ccccc2C(=O)O)c2nc(-c3cnn(-c4ccc(C#N)cc4)c3)cc(=O)n2c1] | (R)-2-((1-(2-(1-(4-cyanophenyl)- 1H-pyrazol-4-yl)-7-methyl-4-oxo- 4H-pyrido[1,2-a]pyrimidin-9- yl)ethyl)amino)benzoic acid |
| 11-241 | 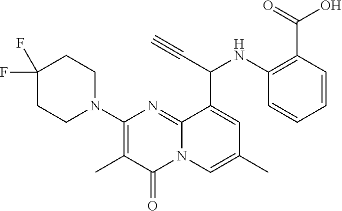 [SMILES: C#CC(Nc1ccccc1C(=O)O)c1cc(C)cn2c(=O)c(C)c(N3CCC(F)(F)CC3)nc12] | 2-((1-(2-(4,4-difluoropiperidin-1- yl)-3,7-dimethyl-4-oxo- pyrido[1,2-a]pyrimidin-9-yl)prop- 2-yn-1-yl)amino)benzoic acid |
| 11-242 | 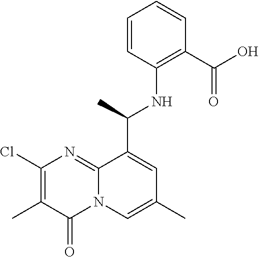 [SMILES: Cc1cc([C@@H](C)Nc2ccccc2C(=O)O)c2nc(Cl)c(C)c(=O)n2c1] | (R)-2-((1-(2-chloro-3,7-dimethyl- 4-oxo-4H-pyrido[1,2-a]pyrimidin- 9-yl)ethyl)amino)benzoic acid |
| 11-243 | 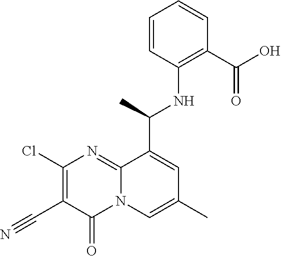 [SMILES: Cc1cc([C@@H](C)Nc2ccccc2C(=O)O)c2nc(Cl)c(C#N)c(=O)n2c1] | (R)-2-((1-(2-chloro-3-cyano-7- methyl-4-oxo- 4H-pyrido[1,2-a]pyrimidin-9- yl)ethyl)amino)benzoic acid |
| 11-244 | 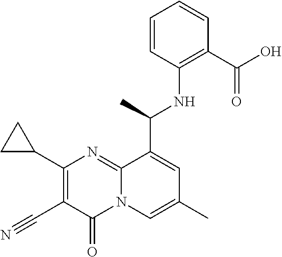 [SMILES: Cc1cc([C@@H](C)Nc2ccccc2C(=O)O)c2nc(C3CC3)c(C#N)c(=O)n2c1] | (R)-2-((1-(3-cyano-2-cyclopropyl- 7-methyl-4-oxo- 4H-pyrido[1,2-a]pyrimidin-9- yl)ethyl)amino)benzoic acid |
| 11-245 | 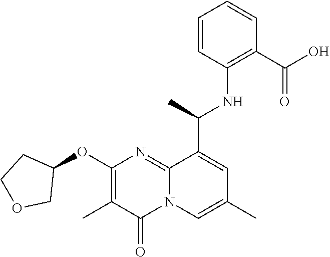 [SMILES: Cc1cc([C@@H](C)Nc2ccccc2C(=O)O)c2nc(O[C@@H]3CCOC3)c(C)c(=O)n2c1] | 2-(((R)-1-(3,7-dimethyl-4-oxo-2- (((R)-tetrahydrofuran-3-yl)oxy)- 4H-pyrido[1,2-a]pyrimidin-9- yl)ethyl)amino)benzoic acid |
| 11-246 | 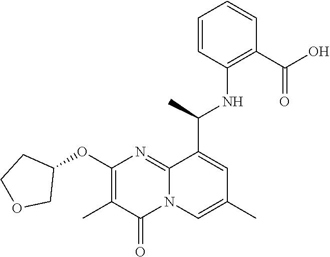 [SMILES: Cc1cc([C@@H](C)Nc2ccccc2C(=O)O)c2nc(O[C@H]3CCOC3)c(C)c(=O)n2c1] | 2-(((R)-1-(3,7-dimethyl-4-oxo-2- (((S)-tetrahydrofuran-3-yl)oxy)- 4H-pyrido[1,2-a]pyrimidin-9- yl)ethyl)amino)benzoic acid |
| 11-247 | 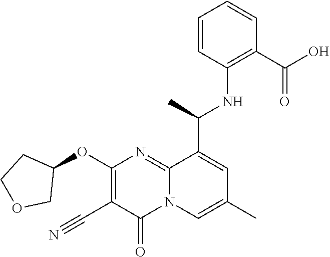 [SMILES: Cc1cc([C@@H](C)Nc2ccccc2C(=O)O)c2nc(O[C@@H]3CCOC3)c(C#N)c(=O)n2c1] | 2-(((R)-1-(3-cyano-7-methyl-4- oxo-2-(((R)-tetrahydrofuran-3- yl)oxy)-4H-pyrido[1,2-a]pyrimidin-9- yl)ethyl)amino)benzoic acid |
| 11-248 | 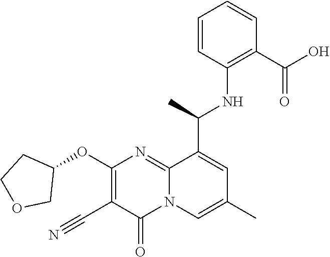 [SMILES: Cc1cc([C@@H](C)Nc2ccccc2C(=O)O)c2nc(O[C@H]3CCOC3)c(C#N)c(=O)n2c1] | 2-(((R)-1-(3-cyano-7-methyl-4- oxo-2-(((S)-tetrahydrofuran-3- yl)oxy)-4H-pyrido[1,2-a]pyrimidin-9- yl)ethyl)amino)benzoic acid |
| 11-249 | 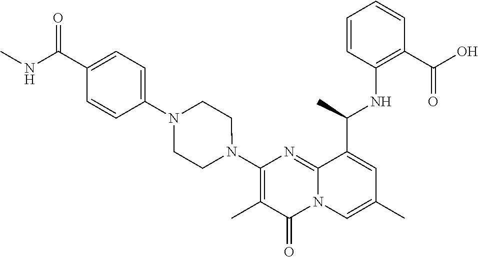 [SMILES: CNC(=O)c1ccc(N2CCN(c3nc4c([C@@H](C)Nc5ccccc5C(=O)O)cc(C)cn4c(=O)c3C)CC2)cc1] | (R)-2-((1-(3,7-dimethyl-2-(4-(4- (methylcarbamoyl)phenyl)piperazin-1- yl)-4-oxo-4H-pyrido[1,2-a]pyrimidin-9- yl)ethyl)amino)benzoic acid |
| 11-250 | 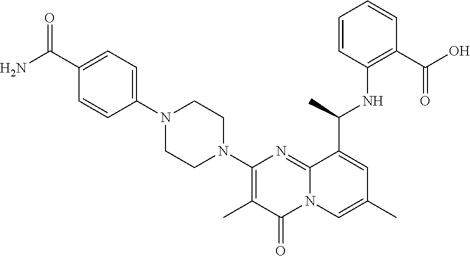 [SMILES: Cc1cc([C@@H](C)Nc2ccccc2C(=O)O)c2nc(N3CCN(c4ccc(C(N)=O)cc4)CC3)c(C)c(=O)n2c1] | (R)-2-((1-(2-(4-(4- carbamoylphenyl)piperazin-1-yl)- 3,7-dimethyl-4-oxo- 4H-pyrido[1,2-a]pyrimidin-9- yl)ethyl)amino)benzoic acid |
| 11-251 | 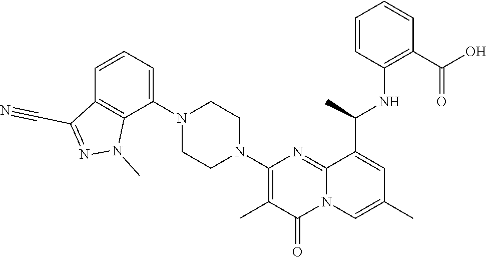 [SMILES: Cc1cc([C@@H](C)Nc2ccccc2C(=O)O)c2nc(N3CCN(c4cccc5c(C#N)nn(C)c45)CC3)c(C)c(=O)n2c1] | (R)-2-((1-(2-(4-(3-cyano-1- methyl-1H-indazol-7- yl)piperazin-1-yl)-3,7-dimethyl-4- oxo-4H-pyrido[1,2-a]pyrimidin-9- yl)ethyl)amino)benzoic acid |
| 11-252 | 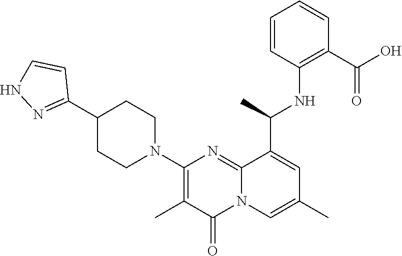 [SMILES: Cc1cc([C@@H](C)Nc2ccccc2C(=O)O)c2nc(N3CCC(c4cc[nH]n4)CC3)c(C)c(=O)n2c1] | (R)-2-((1-(2-(4-(1H-pyrazol-3- yl)piperidin-1-yl)-3,7-dimethyl-4- oxo-4H-pyrido[1,2-a]pyrimidin-9- yl)ethyl)amino)benzoic acid |
| 11-253 | 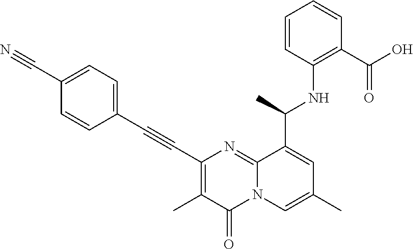 [SMILES: Cc1cc([C@@H](C)Nc2ccccc2C(=O)O)c2nc(C#Cc3ccc(C#N)cc3)c(C)c(=O)n2c1] | (R)-2-((1-(2-((4- cyanophenyl)ethynyl)-3,7- dimethyl-4-oxo-4H-pyrido [1,2-a]pyrimidin-9- yl)ethyl)amino)benzoic acid |
| 11-254 | 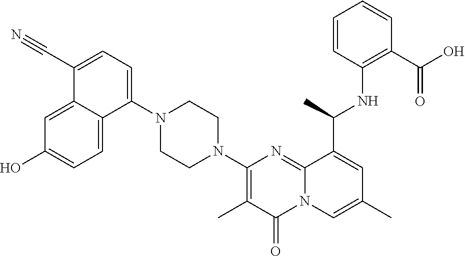 [SMILES: Cc1cc([C@@H](C)Nc2ccccc2C(=O)O)c2nc(N3CCN(c4ccc(C#N)c5cc(O)ccc45)CC3)c(C)c(=O)n2c1] | (R)-2-((1-(2-(4-(4-cyano-6- hydroxynaphthalen-1- yl)piperazin-1-yl)-3,7-dimethyl-4- oxo-4H-pyrido[1,2-a]pyrimidin-9- yl)ethyl)amino)benzoic acid |
| 11-255 | 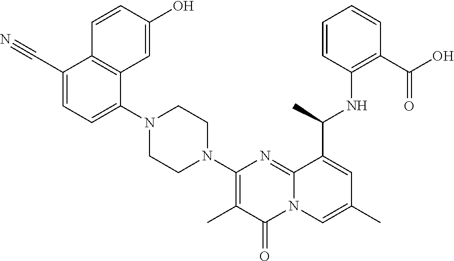 [SMILES: Cc1cc([C@@H](C)Nc2ccccc2C(=O)O)c2nc(N3CCN(c4ccc(C#N)c5ccc(O)cc45)CC3)c(C)c(=O)n2c1] | (R)-2-((1-(2-(4-(4-cyano-7- hydroxynaphthalen-1- yl)piperazin-1-yl)-3,7-dimethyl-4- oxo-4H-pyrido[1,2-a]pyrimidin-9- yl)ethyl)amino)benzoic acid |
| 11-256 | 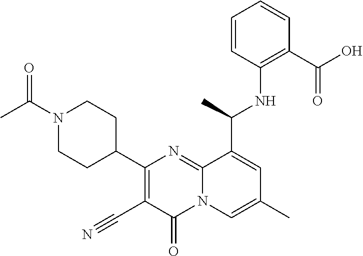 [SMILES: CC(=O)N1CCC(c2nc3c([C@@H](C)Nc4ccccc4C(=O)O)cc(C)cn3c(=O)c2C#N)CC1] | (R)-2-((1-(2-(1-acetylpiperidin-4- yl)-3-cyano-7-methyl-4-oxo- 4H-pyrido[1,2-a]pyrimidin-9- yl)ethyl)amino)benzoic acid |
| 11-257 | 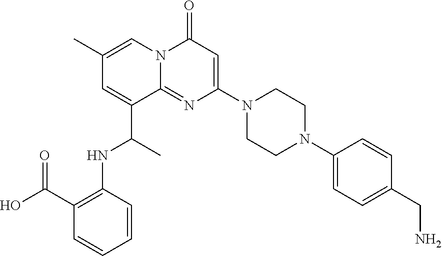 [SMILES: Cc1cc(C(C)Nc2ccccc2C(=O)O)c2nc(N3CCN(c4ccc(CN)cc4)CC3)cc(=O)n2c1] | 2-((1-(2-(4-(4- (aminomethyl)phenyl)piperazin- 1-yl)-7-methyl-4-oxo- 4H-pyrido[1,2-a]pyrimidin-9- yl)ethyl)amino)benzoic acid |
| 11-258 | 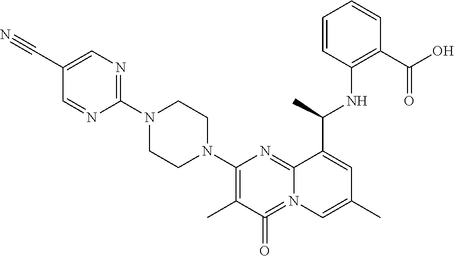 | (R)-2-((1-(2-(4-(5- cyanopyrimidin-2-yl)piperazin-1- yl)-3,7-dimethyl-4-oxo- 4H-pyrido[1,2-a]pyrimidin-9- yl)ethyl)amino)benzoic acid |
| 11-259 | 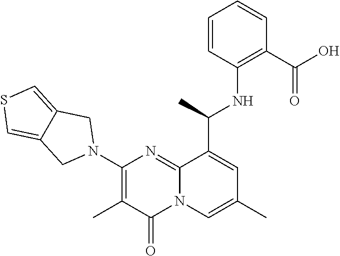 [SMILES: Cc1cc([C@@H](C)Nc2ccccc2C(=O)O)c2nc(N3Cc4cscc4C3)c(C)c(=O)n2c1] | 2-(((1R)-1-(3,7-dimethyl-4-oxo-2- (tetrahydro-1H-thieno[3,4- c]pyrrol-5(3H)-yl)- 4H-pyrido[1,2-a]pyrimidin-9- yl)ethyl)amino)benzoic acid |
| 11-260 | 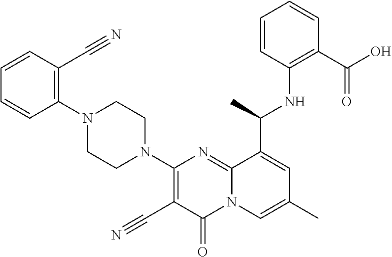 [SMILES: Cc1cc([C@@H](C)Nc2ccccc2C(=O)O)c2nc(N3CCN(c4ccccc4C#N)CC3)c(C#N)c(=O)n2c1] | (R)-2-((1-(3-cyano-2-(4-(2- cyanophenyl)piperazin-1-yl)-7- methyl-4-oxo- 4H-pyrido[1,2-a]pyrimidin-9- yl)ethyl)amino)benzoic acid |
| 11-261 | 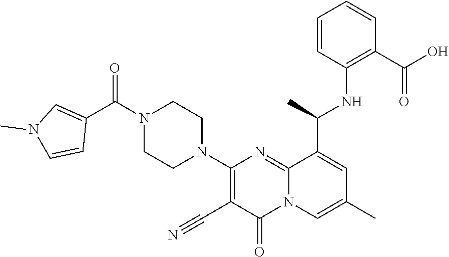 [SMILES: Cc1cc([C@@H](C)Nc2ccccc2C(=O)O)c2nc(N3CCN(C(=O)c4ccn(C)c4)CC3)c(C#N)c(=O)n2c1] | (R)-2-((1-(3-cyano-7-methyl-2-(4- (1-methyl-1H-pyrazole-4- carbonyl)piperazin-1-yl)-4-oxo- 4H-pyrido[1,2-a]pyrimidin-9- yl)ethyl)amino)benzoic acid |
| 11-262 | 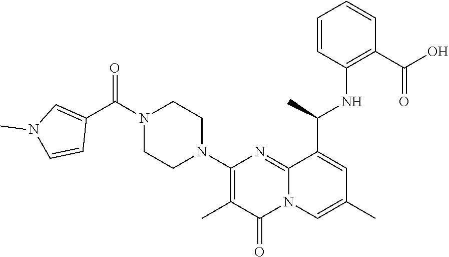 [SMILES: Cc1cc([C@@H](C)Nc2ccccc2C(=O)O)c2nc(N3CCN(C(=O)c4ccn(C)c4)CC3)c(C)c(=O)n2c1] | (R)-2-((1-(3,7-dimethyl-2-(4-(1- methyl-1H-pyrazole-4- carbonyl)piperazin-1-yl)-4-oxo- 4H-pyrido[1,2-a]pyrimidin-9- yl)ethyl)amino)benzoic acid |
| 11-263 | 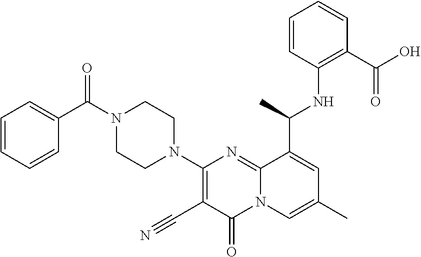 [SMILES: Cc1cc([C@@H](C)Nc2ccccc2C(=O)O)c2nc(N3CCN(C(=O)c4ccccc4)CC3)c(C#N)c(=O)n2c1] | (R)-2-((1-(2-(4-benzoylpiperazin- 1-yl)-3-cyano-7-methyl-4-oxo- 4H-pyrido[1,2-a]pyrimidin-9- yl)ethyl)amino)benzoic acid |
| 11-264 | 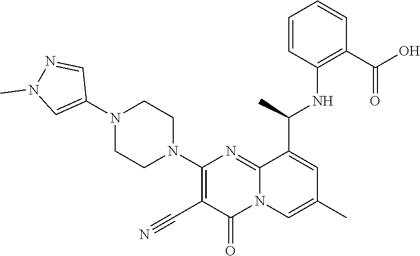 [SMILES: Cc1cc([C@@H](C)Nc2ccccc2C(=O)O)c2nc(N3CCN(c4cnn(C)c4)CC3)c(C#N)c(=O)n2c1] | (R)-2-((1-(3-cyano-7-methyl-2-(4- (1-methyl-1H-pyrazol-4- yl)piperazin-1-yl)-4-oxo- 4H-pyrido[1,2-a]pyrimidin-9- yl)ethyl)amino)benzoic acid |
| 11-265 | 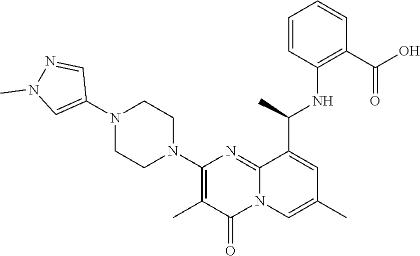 [SMILES: Cc1cc([C@@H](C)Nc2ccccc2C(=O)O)c2nc(N3CCN(c4cnn(C)c4)CC3)c(C)c(=O)n2c1] | (R)-2-((1-(3,7-dimethyl-2-(4-(1- methyl-1H-pyrazol-4- yl)piperazin-1-yl)-4-oxo- 4H-pyrido[1,2-a]pyrimidin-9- yl)ethyl)amino)benzoic acid |
| 11-266 | 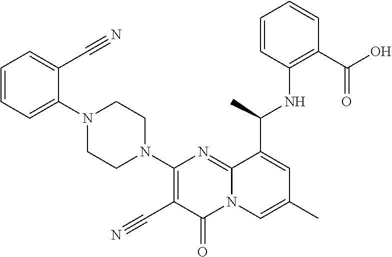 [SMILES: Cc1cc([C@@H](C)Nc2ccccc2C(=O)O)c2nc(N3CCN(c4ccccc4C#N)CC3)c(C#N)c(=O)n2c1] | (R)-2-((1-(3-cyano-7-methyl-4- oxo-2-(4-(o-tolyl)piperazin-1-yl)- 4H-pyrido[1,2-a]pyrimidin-9- yl)ethyl)amino)benzoic acid |
| 11-267 | 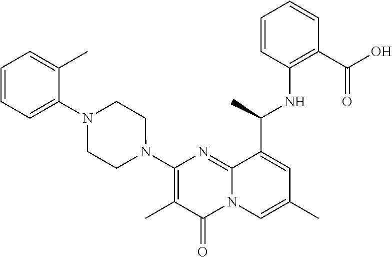 [SMILES: Cc1cc([C@@H](C)Nc2ccccc2C(=O)O)c2nc(N3CCN(c4ccccc4C)CC3)c(C)c(=O)n2c1] | (R)-2-((1-(3,7-dimethyl-4-oxo-2- (4-(o-tolyl)piperazin-1-yl)- 4H-pyrido[1,2-a]pyrimidin-9- yl)ethyl)amino)benzoic acid |
| 11-268 | 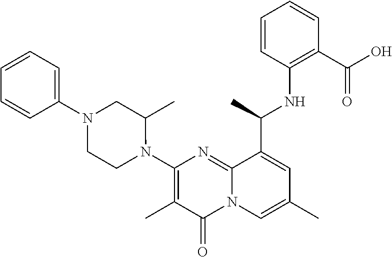 [SMILES: Cc1cc([C@@H](C)Nc2ccccc2C(=O)O)c2nc(N3CCN(c4ccccc4)CC3C)c(C)c(=O)n2c1] | 2-(((1R)-1-(3,7-dimethyl-2-(2- methyl-4-phenylpiperazin-1-yl)-4- oxo-4H-pyrido[1,2-a]pyrimidin-9- yl)ethyl)amino)benzoic acid |
| 11-269 | 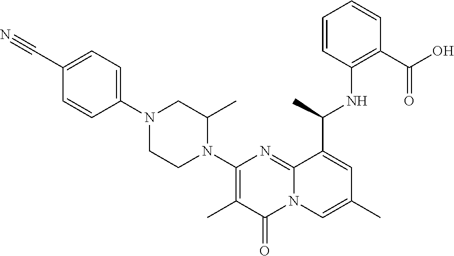 [SMILES: Cc1cc([C@@H](C)Nc2ccccc2C(=O)O)c2nc(N3CCN(c4ccc(C#N)cc4)CC3C)c(C)c(=O)n2c1] | 2-(((1R)-1-(2-(4-(4-cyanophenyl)- 2-methylpiperazin-1-yl)-3,7- dimethyl-4-oxo- 4H-pyrido[1,2-a]pyrimidin-9- yl)ethyl)amino)benzoic acid |
| 11-270 | 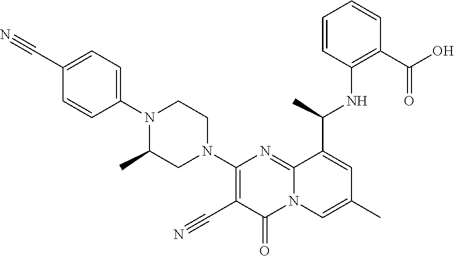 [SMILES: Cc1cc([C@@H](C)Nc2ccccc2C(=O)O)c2nc(N3CCN(c4ccc(C#N)cc4)[C@H](C)C3)c(C#N)c(=O)n2c1] | 2-(((R)-1-(3-cyano-2-((R)-4-(4- cyanophenyl)-3-methylpiperazin- 1-yl)-7-methyl-4-oxo- 4H-pyrido[1,2-a]pyrimidin-9- yl)ethyl)amino)benzoic acid |
| 11-271 | 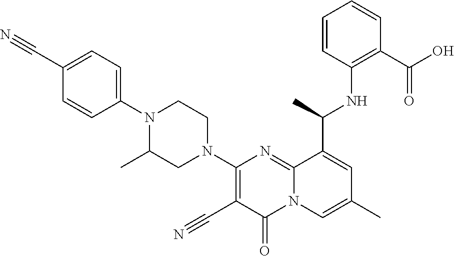 [SMILES: Cc1cc([C@@H](C)Nc2ccccc2C(=O)O)c2nc(N3CCN(c4ccc(C#N)cc4)C(C)C3)c(C#N)c(=O)n2c1] | 2-(((1R)-1-(3-cyano-2-(4-(4- cyanophenyl)-3-methylpiperazin- 1-yl)-7-methyl-4-oxo- 4H-pyrido[1,2-a]pyrimidin-9- yl)ethyl)amino)benzoic acid |
| 11-272 | 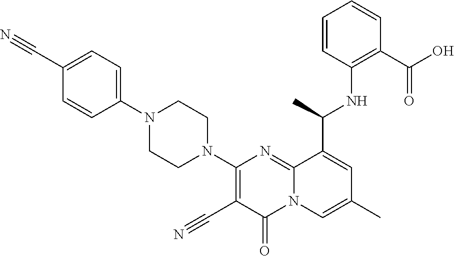 [SMILES: Cc1cc([C@@H](C)Nc2ccccc2C(=O)O)c2nc(N3CCN(c4ccc(C#N)cc4)CC3)c(C#N)c(=O)n2c1] | (R)-2-((1-(3-cyano-2-(4-(4- cyanophenyl)piperazin-1-yl)-7- methyl-4-oxo- 4H-pyrido[1,2-a]pyrimidin-9- yl)ethyl)amino)benzoic acid |
| 11-273 | 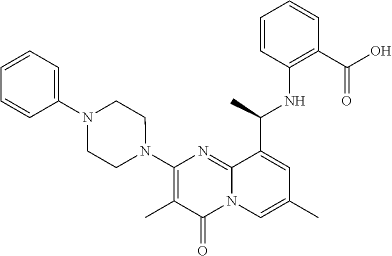 [SMILES: Cc1cc([C@@H](C)Nc2ccccc2C(=O)O)c2nc(N3CCN(c4ccccc4)CC3)c(C)c(=O)n2c1] | (R)-2-((1-(3,7-dimethyl-4-oxo-2- (4-phenylpiperazin-1-yl)- 4H-pyrido[1,2-a]pyrimidin-9- yl)ethyl)amino)benzoic acid |
| 11-274 | 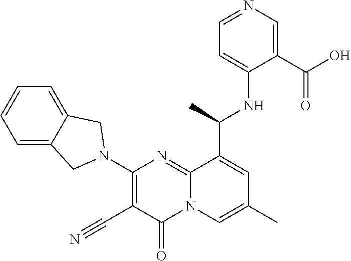 [SMILES: Cc1cc([C@@H](C)Nc2ccncc2C(=O)O)c2nc(N3Cc4ccccc4C3)c(C#N)c(=O)n2c1] | (R)-4-((1-(3-cyano-2-(isoindolin- 2-yl)-7-methyl-4-oxo- 4H-pyrido[1,2-a]pyrimidin-9- yl)ethyl)amino)nicotinic acid |
| 11-275 | 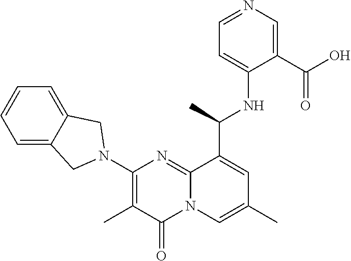 [SMILES: Cc1cc([C@@H](C)Nc2ccncc2C(=O)O)c2nc(N3Cc4ccccc4C3)c(C)c(=O)n2c1] | (R)-4-((1-(2-(isoindolin-2-yl)-3,7- dimethyl-4-oxo-4H-pyrido[1,2- a]pyrimidin-9- yl)ethyl)amino)nicotinic acid |
| 11-276 | 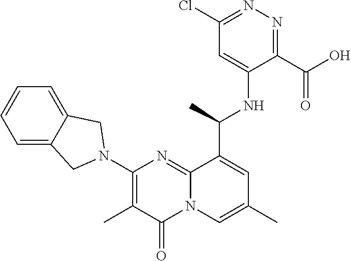 [SMILES: Cc1cc([C@@H](C)Nc2cc(Cl)nnc2C(=O)O)c2nc(N3Cc4ccccc4C3)c(C)c(=O)n2c1] | (R)-6-chloro-4-((1-(2-(isoindolin- 2-yl)-3,7-dimethyl-4-oxo-4H- pyrido[1,2-a]pyrimidin-9- yl)ethyl)amino)pyridazine-3- carboxylic acid |
| 11-277 | 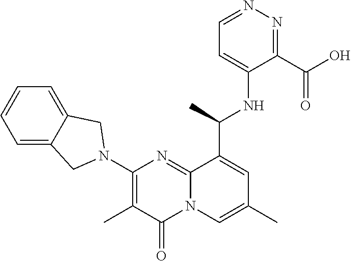 [SMILES: Cc1cc([C@@H](C)Nc2ccnnc2C(=O)O)c2nc(N3Cc4ccccc4C3)c(C)c(=O)n2c1] | (R)-4-((1-(2-(isoindolin-2-yl)-3,7- dimethyl-4-oxo-4H-pyrido[1,2- a]pyrimidin-9- yl)ethyl)amino)pyridazine-3- carboxylic acid |
| 11-278 | 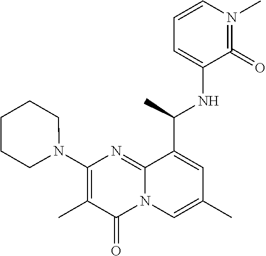 | (R)-3,7-dimethyl-9-(1-((1-methyl- 2-oxo-1,2-dihydropyridin-3- yl)amino)ethyl)-2-(piperidin-1-yl)- 4H-pyrido[1,2-a]pyrimidin-4-one |
| 11-279 | 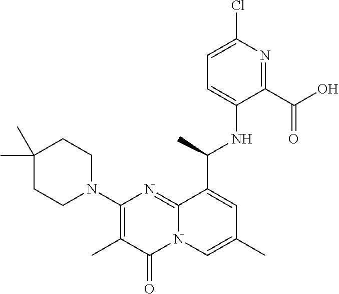 [SMILES: Cc1cc([C@@H](C)Nc2ccc(Cl)nc2C(=O)O)c2nc(N3CCC(C)(C)CC3)c(C)c(=O)n2c1] | (R)-6-chloro-3-((1-(2-(4,4- dimethylpiperidin-1-yl)-3,7- dimethyl-4-oxo-4H-pyrido[1,2- a]pyrimidin-9- yl)ethyl)amino)picolinic acid |
| 11-280 | 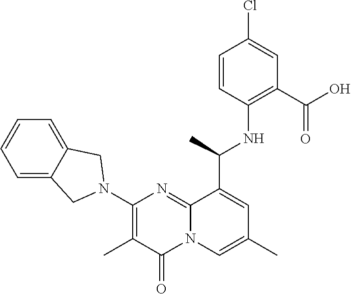 [SMILES: Cc1cc([C@@H](C)Nc2ccc(Cl)cc2C(=O)O)c2nc(N3Cc4ccccc4C3)c(C)c(=O)n2c1] | (R)-5-chloro-2-((1-(2-(isoindolin- 2-yl)-3,7-dimethyl-4-oxo-4H- pyrido[1,2-a]pyrimidin-9- yl)ethyl)amino)benzoic acid |
| 11-281 | 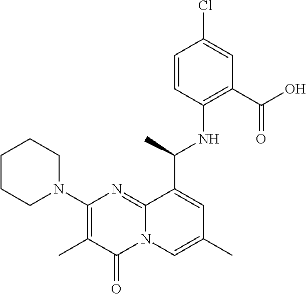 [SMILES: Cc1cc([C@@H](C)Nc2ccc(Cl)cc2C(=O)O)c2nc(N3CCCCC3)c(C)c(=O)n2c1] | (R)-5-chloro-2-((1-(3,7-dimethyl- 4-oxo-2-(piperidin-1-yl)- 4H-pyrido[1,2-a]pyrimidin-9- yl)ethyl)amino)benzoic acid |
| 11-282 | 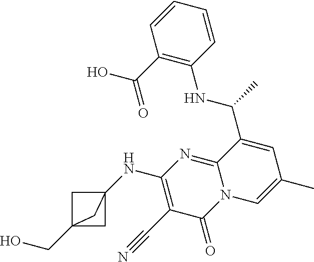 [SMILES: Cc1cc([C@@H](C)Nc2ccccc2C(=O)O)c2nc(NC34CC(CO)(C3)C4)c(C#N)c(=O)n2c1] | (R)-2-((1-(3-cyano-2-((3- (hydroxymethyl)bicyclo[1.1.1]pentan- 1-yl)amino)-7-methyl-4-oxo- 4H-pyrido[1,2-a]pyrimidin-9- yl)ethyl)amino)benzoic acid |
| 11-283 | 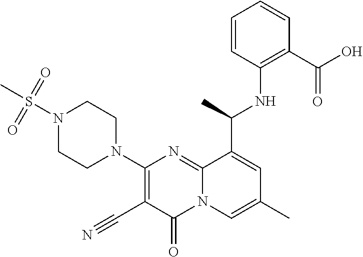 [SMILES: Cc1cc([C@@H](C)Nc2ccccc2C(=O)O)c2nc(N3CCN(S(C)(=O)=O)CC3)c(C#N)c(=O)n2c1] | (R)-2-((1-(3-cyano-7-methyl-2-(4- (methylsulfonyl)piperazin-1-yl)-4- oxo-4H-pyrido[1,2-a]pyrimidin-9- yl)ethyl)amino)benzoic acid |
| 11-284 | 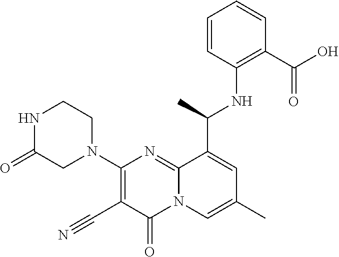 [SMILES: Cc1cc([C@@H](C)Nc2ccccc2C(=O)O)c2nc(N3CCNC(=O)C3)c(C#N)c(=O)n2c1] | (R)-2-((1-(3-cyano-7-methyl-4- oxo-2-(3-oxopiperazin-1-yl)- 4H-pyrido[1,2-a]pyrimidin-9- yl)ethyl)amino)benzoic acid |
| 11-285 | 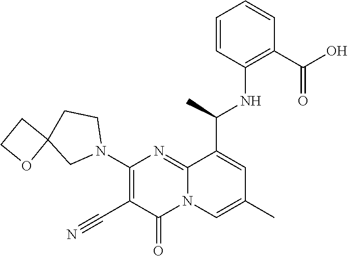 [SMILES: Cc1cc([C@@H](C)Nc2ccccc2C(=O)O)c2nc(N3CCC4(CCO4)C3)c(C#N)c(=O)n2c1] | 2-(((1R)-1-(3-cyano-7-methyl-4- oxo-2-(1-oxa-6- azaspiro[3.4]octan-6-yl)- 4H-pyrido[1,2-a]pyrimidin-9- yl)ethyl)amino)benzoic acid |
| 11-286 | 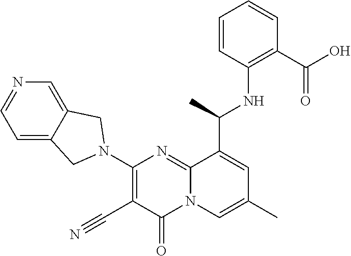 [SMILES: Cc1cc([C@@H](C)Nc2ccccc2C(=O)O)c2nc(N3Cc4ccncc4C3)c(C#N)c(=O)n2c1] | (R)-2-((1-(3-cyano-2-(1,3- dihydro-2H-pyrrolo[3,4-c]pyridin- 2-yl)-7-methyl-4-oxo- 4H-pyrido[1,2-a]pyrimidin-9- yl)ethyl)amino)benzoic acid |
| 11-287 | 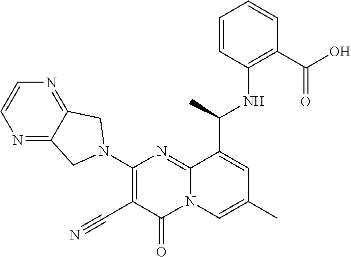 [SMILES: Cc1cc([C@@H](C)Nc2ccccc2C(=O)O)c2nc(N3Cc4nccnc4C3)c(C#N)c(=O)n2c1] | (R)-2-((1-(3-cyano-2-(5,7- dihydro-6H-pyrrolo[3,4-b]pyridin- 6-yl)-7-methyl-4-oxo- 4H-pyrido[1,2-a]pyrimidin-9- yl)ethyl)amino)benzoic acid |
| 11-288 | 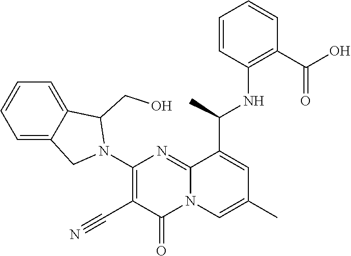 [SMILES: Cc1cc([C@@H](C)Nc2ccccc2C(=O)O)c2nc(N3Cc4ccccc4C3CO)c(C#N)c(=O)n2c1] | 2-(((1R)-1-(3-cyano-2-(1- (hydroxymethyl)isoindolin-2-yl)- 7-methyl-4-oxo- 4H-pyrido[1,2-a]pyrimidin-9- yl)ethyl)amino)benzoic acid |
| 11-289 | 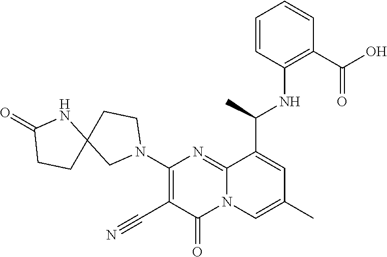 [SMILES: Cc1cc([C@@H](C)Nc2ccccc2C(=O)O)c2nc(N3CCC4(CCC(=O)N4)C3)c(C#N)c(=O)n2c1] | 2-(((1R)-1-(3-cyano-7-methyl-4- oxo-2-(2-oxo-1,7- diazaspiro[4.4]nonan-7-yl)- 4H-pyrido[1,2-a]pyrimidin-9- yl)ethyl)amino)benzoic acid |
| 11-290 | 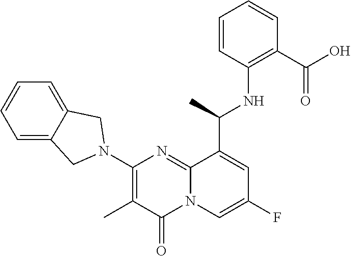 [SMILES: Cc1c(N2Cc3ccccc3C2)nc2c([C@@H](C)Nc3ccccc3C(=O)O)cc(F)cn2c1=O] | (R)-2-((1-(7-fluoro-2-(isoindolin- 2-yl)-3-methyl-4-oxo- 4H-pyrido[1,2-a]pyrimidin-9- yl)ethyl)amino)benzoic acid |
| 11-291 | 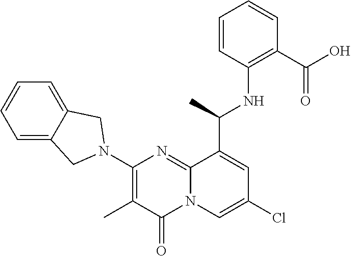 [SMILES: Cc1c(N2Cc3ccccc3C2)nc2c([C@@H](C)Nc3ccccc3C(=O)O)cc(Cl)cn2c1=O] | (R)-2-((1-(7-chloro-2-(isoindolin- 2-yl)-3-methyl-4-oxo- 4H-pyrido[1,2-a]pyrimidin-9- yl)ethyl)amino)benzoic acid |
| 11-292 | 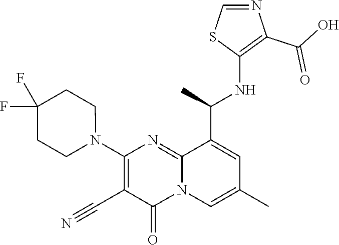 [SMILES: Cc1cc([C@@H](C)Nc2scnc2C(=O)O)c2nc(N3CCC(F)(F)CC3)c(C#N)c(=O)n2c1] | (R)-5-((1-(3-cyano-2-(4,4- difluoropiperidin-1-yl)-7-methyl- 4-oxo-4H-pyrido[1,2-a]pyrimidin-9- yl)ethyl)amino)thiazole-4- carboxylic acid |
| 11-293 | 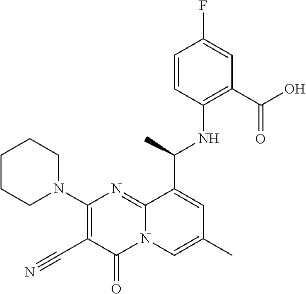 [SMILES: Cc1cc([C@@H](C)Nc2ccc(F)cc2C(=O)O)c2nc(N3CCCCC3)c(C#N)c(=O)n2c1] | (R)-2-((1-(3-cyano-7-methyl-4- oxo-2-(piperidin-1-yl)-4H- pyrido[1,2-a]pyrimidin-9- yl)ethyl)amino)-5-fluorobenzoic acid |
| 11-294 | 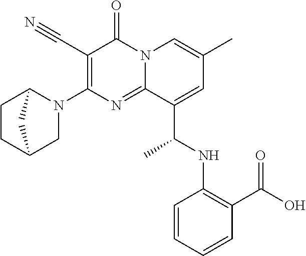 [SMILES: Cc1cc([C@@H](C)Nc2ccccc2C(=O)O)c2nc(N3C[C@H]4CC[C@@H]3C4)c(C#N)c(=O)n2c1] | 2-(((R)-1-(2-((1R,4S)-2- azabicyclo[2.2.1]heptan-2-yl)-3- cyano-7-methyl-4-oxo- 4H-pyrido[1,2-a]pyrimidin-9- yl)ethyl)amino)benzoic acid |
| 11-295 | 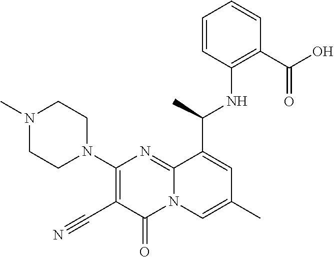 [SMILES: Cc1cc([C@@H](C)Nc2ccccc2C(=O)O)c2nc(N3CCN(C)CC3)c(C#N)c(=O)n2c1] | (R)-2-((1-(3-cyano-7-methyl-2-(4- methylpiperazin-1-yl)-4-oxo- 4H-pyrido[1,2-a]pyrimidin-9- yl)ethyl)amino)benzoic acid |
| 11-296 | 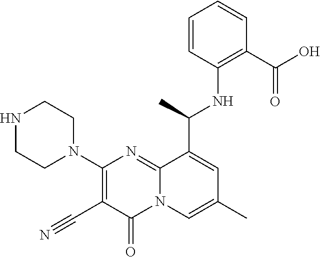 [SMILES: Cc1cc([C@@H](C)Nc2ccccc2C(=O)O)c2nc(N3CCNCC3)c(C#N)c(=O)n2c1] | (R)-2-((1-(3-cyano-7-methyl-4- oxo-2-(piperazin-1-yl)- 4H-pyrido[1,2-a]pyrimidin-9- yl)ethyl)amino)benzoic acid |
| 11-297 | 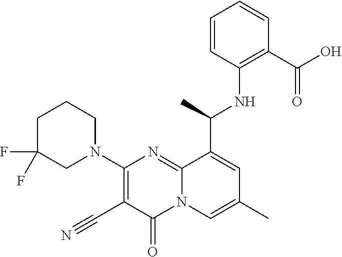 [SMILES: Cc1cc([C@@H](C)Nc2ccccc2C(=O)O)c2nc(N3CCCC(F)(F)C3)c(C#N)c(=O)n2c1] | (R)-2-((1-(3-cyano-2-(3,3- difluoropiperidin-1-yl)-7-methyl- 4-oxo-4H-pyrido[1,2-a]pyrimidin-9- yl)ethyl)amino)benzoic acid |
| 11-298 | 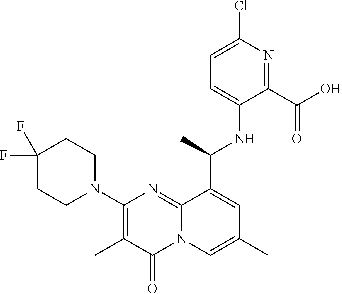 [SMILES: Cc1cc([C@@H](C)Nc2ccc(Cl)nc2C(=O)O)c2nc(N3CCC(F)(F)CC3)c(C)c(=O)n2c1] | (R)-6-chloro-3-((1-(2-(4,4- difluoropiperidin-1-yl)-3,7- dimethyl-4-oxo-4H-pyrido[1,2- a]pyrimidin-9- yl)ethyl)amino)picolinic acid |
| 11-299 | 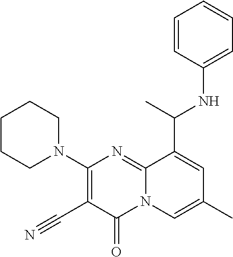 [SMILES: Cc1cc(C(C)Nc2ccccc2)c2nc(N3CCCCC3)c(C#N)c(=O)n2c1] | 7-methyl-4-oxo-9-(1- (phenylamino)ethyl)-2-(piperidin- 1-yl)-4H-pyrido[1,2-a]pyrimidine- 3-carbonitrile |
| 11-300 | 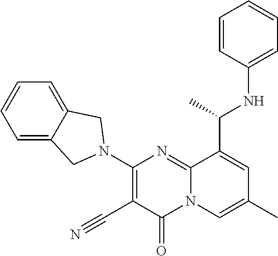 [SMILES: Cc1cc([C@H](C)Nc2ccccc2)c2nc(N3Cc4ccccc4C3)c(C#N)c(=O)n2c1] | (S)-2-(isoindolin-2-yl)-7-methyl- 4-oxo-9-(1-(phenylamino)ethyl)- 4H-pyrido[1,2-a]pyrimidine-3- carbonitrile |
| 11-301 | 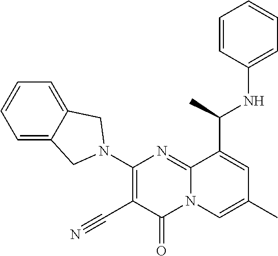 [SMILES: Cc1cc([C@@H](C)Nc2ccccc2)c2nc(N3Cc4ccccc4C3)c(C#N)c(=O)n2c1] | (R)-2-(isoindolin-2-yl)-7-methyl- 4-oxo-9-(1-(phenylamino)ethyl)- 4H-pyrido[1,2-a]pyrimidine-3- carbonitrile |
| 11-302 | 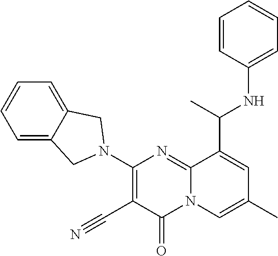 [SMILES: Cc1cc(C(C)Nc2ccccc2)c2nc(N3Cc4ccccc4C3)c(C#N)c(=O)n2c1] | 2-(isoindolin-2-yl)-7-methyl-4- oxo-9-(1-(phenylamino)ethyl)- 4H-pyrido[1,2-a]pyrimidine-3- carbonitrile |
| 11-303 | 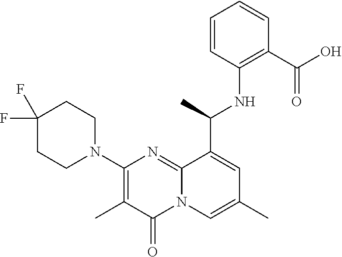 [SMILES: Cc1cc([C@@H](C)Nc2ccccc2C(=O)O)c2nc(N3CCC(F)(F)CC3)c(C)c(=O)n2c1] | (R)-2-((1-(2-(4,4- difluoropiperidin-1-yl)-3,7- dimethyl-4-oxo-4H-pyrido [1,2-a]pyrimidin-9- yl)ethyl)amino)benzoic acid |
| 11-304 | 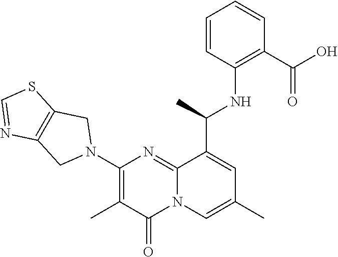 [SMILES: Cc1cc([C@@H](C)Nc2ccccc2C(=O)O)c2nc(N3Cc4ncsc4C3)c(C)c(=O)n2c1] | (R)-2-((1-(2-(4,6-dihydro-5H- pyrrolo[3,4-d]thiazol-5-yl)-3,7- dimethyl-4-oxo-4H-pyrido[1,2-a]pyrimidin-9- yl)ethyl)amino)benzoic acid |
| 11-305 | 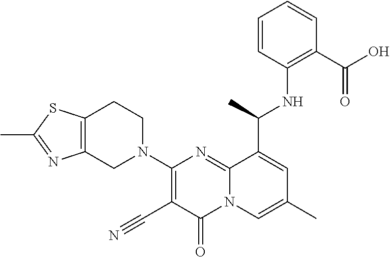 [SMILES: Cc1cc([C@@H](C)Nc2ccccc2C(=O)O)c2nc(N3CCc4sc(C)nc4C3)c(C#N)c(=O)n2c1] | (R)-2-((1-(3-cyano-7-methyl-2-(2- methyl-6,7-dihydrothiazolo[4,5- c]pyridin-5(4H)-yl)-4-oxo- 4H-pyrido[1,2-a]pyrimidin-9- yl)ethyl)amino)benzoic acid |
| 11-306 | 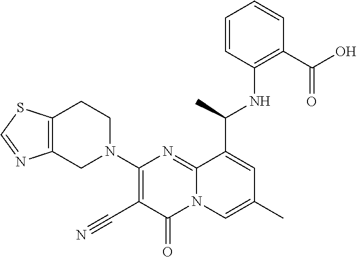 [SMILES: Cc1cc([C@@H](C)Nc2ccccc2C(=O)O)c2nc(N3CCc4scnc4C3)c(C#N)c(=O)n2c1] | (R)-2-((1-(3-cyano-2-(6,7- dihydrothiazolo[4,5-c]pyridin- 5(4H)-yl)-7-methyl-4-oxo- 4H-pyrido[1,2-a]pyrimidin-9- yl)ethyl)amino)benzoic acid |
| 11-307 | 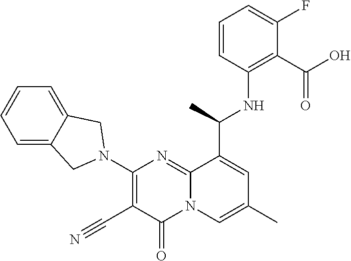 [SMILES: Cc1cc([C@@H](C)Nc2cccc(F)c2C(=O)O)c2nc(N3Cc4ccccc4C3)c(C#N)c(=O)n2c1] | (R)-2-((1-(3-cyano-2-(isoindolin- 2-yl)-7-methyl-4-oxo-4H- pyrido[1,2-a]pyrimidin-9- yl)ethyl)amino)-6-fluorobenzoic acid |
| 11-308 | 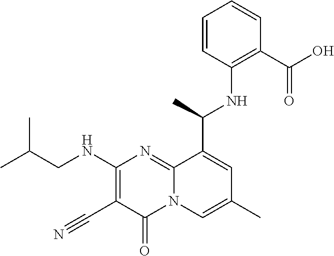 [SMILES: Cc1cc([C@@H](C)Nc2ccccc2C(=O)O)c2nc(NCC(C)C)c(C#N)c(=O)n2c1] | (R)-2-((1-(3-cyano-2- (isobutylamino)-7-methyl-4-oxo- 4H-pyrido[1,2-a]pyrimidin-9- yl)ethyl)amino)benzoic acid |
| 11-309 | 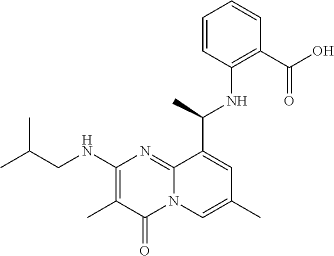 [SMILES: Cc1cc([C@@H](C)Nc2ccccc2C(=O)O)c2nc(NCC(C)C)c(C)c(=O)n2c1] | (R)-2-((1-(2-(isobutylamino)-3,7- dimethyl-4-oxo- 4H-pyrido[1,2-a]pyrimidin-9- yl)ethyl)amino)benzoic acid |
| 11-310 | 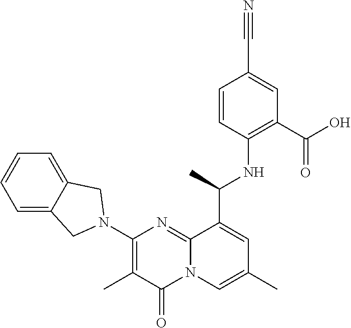 [SMILES: Cc1cc([C@@H](C)Nc2ccc(C#N)cc2C(=O)O)c2nc(N3Cc4ccccc4C3)c(C)c(=O)n2c1] | (R)-5-cyano-2-((1-(2-(isoindolin- 2-yl)-3,7-dimethyl-4-oxo- 4H-pyrido[1,2-a]pyrimidin-9- yl)ethyl)amino)benzoic acid |
| 11-311 | 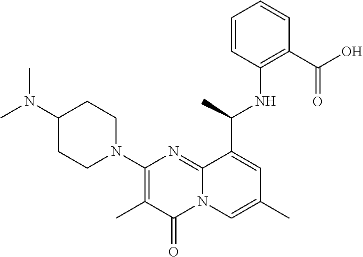 [SMILES: Cc1cc([C@@H](C)Nc2ccccc2C(=O)O)c2nc(N3CCC(N(C)C)CC3)c(C)c(=O)n2c1] | (R)-2-((1-(2-(4- (dimethylamino)piperidin-1-yl)- 3,7-dimethyl-4-oxo- 4H-pyrido[1,2-a]pyrimidin-9- yl)ethyl)amino)benzoic acid |
| 11-312 | 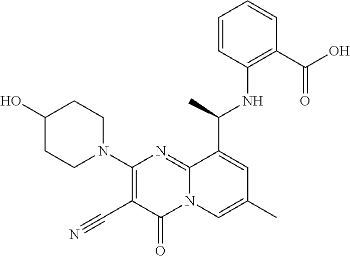 [SMILES: Cc1cc([C@@H](C)Nc2ccccc2C(=O)O)c2nc(N3CCC(O)CC3)c(C#N)c(=O)n2c1] | (R)-2-((1-(3-cyano-2-(4- hydroxypiperidin-1-yl)-7-methyl- 4-oxo-4H-pyrido[1,2-a]pyrimidin-9- yl)ethyl)amino)benzoic acid |
| 11-313 | 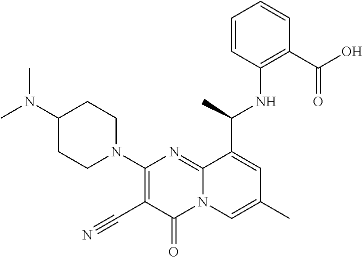 [SMILES: Cc1cc([C@@H](C)Nc2ccccc2C(=O)O)c2nc(N3CCC(N(C)C)CC3)c(C#N)c(=O)n2c1] | (R)-2-((1-(3-cyano-2-(4- (dimethylamino)piperidin-1-yl)-7- methyl-4-oxo- 4H-pyrido[1,2-a]pyrimidin-9- yl)ethyl)amino)benzoic acid |
| 11-314 | 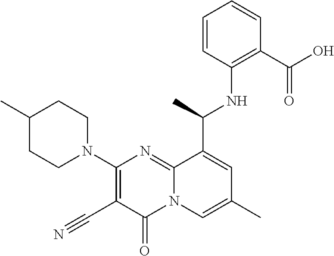 [SMILES: Cc1cc([C@@H](C)Nc2ccccc2C(=O)O)c2nc(N3CCC(C)CC3)c(C#N)c(=O)n2c1] | (R)-2-((1-(3-cyano-7-methyl-2-(4- methylpiperidin-1-yl)-4-oxo- 4H-pyrido[1,2-a]pyrimidin-9- yl)ethyl)amino)benzoic acid |
| 11-315 | 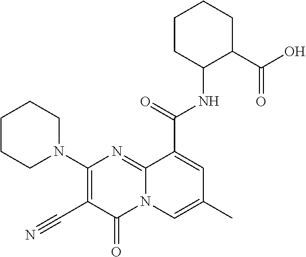 [SMILES: Cc1cc(C(=O)NC2CCCCC2C(=O)O)c2nc(N3CCCCC3)c(C#N)c(=O)n2c1] | 2-(3-cyano-7-methyl-4-oxo-2- (piperidin-1-yl)-4H-pyrido[1,2- a]pyrimidine-9- carboxamido)cyclohexane-1- carboxylic acid |
| 11-316 | 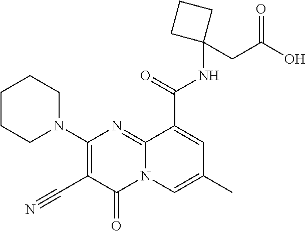 [SMILES: Cc1cc(C(=O)NC2(CC(=O)O)CCC2)c2nc(N3CCCCC3)c(C#N)c(=O)n2c1] | 2-(1-(3-cyano-7-methyl-4-oxo-2- (piperidin-1-yl)-4H-pyrido[1,2- a]pyrimidine-9- carboxamido)cyclobutyl)acetic acid |
| 11-317 | 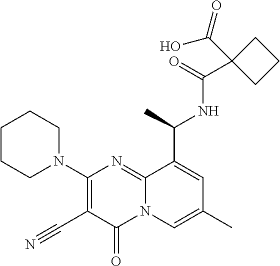 [SMILES: Cc1cc([C@@H](C)NC(=O)C2(C(=O)O)CCC2)c2nc(N3CCCCC3)c(C#N)c(=O)n2c1] | (R)-1-((1-(3-cyano-7-methyl-4- oxo-2-(piperidin-1-yl)-4H- pyrido[1,2-a]pyrimidin-9- yl)ethyl)carbamoyl)cyclobutane- 1-carboxylic acid |
| 11-318 | 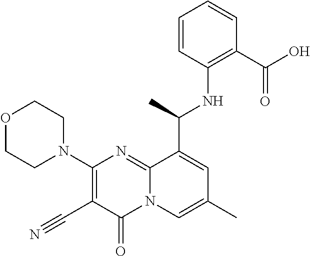 [SMILES: Cc1cc([C@@H](C)Nc2ccccc2C(=O)O)c2nc(N3CCOCC3)c(C#N)c(=O)n2c1] | (R)-2-((1-(3-cyano-7-methyl-2- morpholino-4-oxo- 4H-pyrido[1,2-a]pyrimidin-9- yl)ethyl)amino)benzoic acid |
| 11-319 | 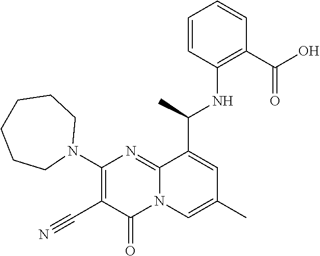 [SMILES: Cc1cc([C@@H](C)Nc2ccccc2C(=O)O)c2nc(N3CCCCCC3)c(C#N)c(=O)n2c1] | (R)-2-((1-(2-(azepan-1-yl)-3- cyano-7-methyl-4-oxo- 4H-pyrido[1,2-a]pyrimidin-9- yl)ethyl)amino)benzoic acid |
| 11-320 | 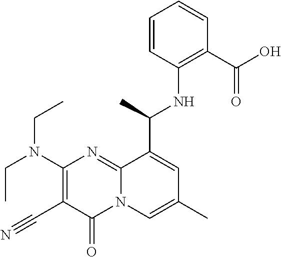 [SMILES: CCN(CC)c1nc2c([C@@H](C)Nc3ccccc3C(=O)O)cc(C)cn2c(=O)c1C#N] | (R)-2-((1-(3-cyano-2- (diethylamino)-7-methyl-4-oxo- 4H-pyrido[1,2-a]pyrimidin-9- yl)ethyl)amino)benzoic acid |
| 11-321 | 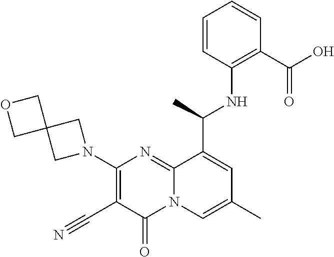 [SMILES: Cc1cc([C@@H](C)Nc2ccccc2C(=O)O)c2nc(N3CC4(COC4)C3)c(C#N)c(=O)n2c1] | (R)-2-((1-(3-cyano-7-methyl-4- oxo-2-(2-oxa-6- azaspiro[3.3]heptan-6-yl)- 4H-pyrido[1,2-a]pyrimidin-9- yl)ethyl)amino)benzoic acid |
| 11-322 | 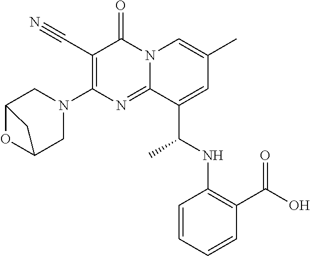 [SMILES: Cc1cc([C@@H](C)Nc2ccccc2C(=O)O)c2nc(N3CC4CC(C3)O4)c(C#N)c(=O)n2c1] | 2-(((1R)-1-(2-(6-oxa-3- azabicyclo[3.1.1]heptan-3-yl)-3- cyano-7-methyl-4-oxo- 4H-pyrido[1,2-a]pyrimidin-9- yl)ethyl)amino)benzoic acid |
| 11-323 | 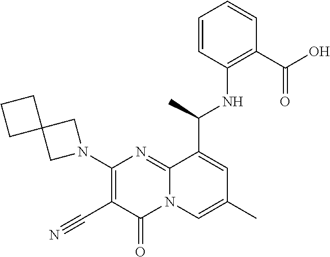 [SMILES: Cc1cc([C@@H](C)Nc2ccccc2C(=O)O)c2nc(N3CC4(CCC4)C3)c(C#N)c(=O)n2c1] | (R)-2-((1-(3-cyano-7-methyl-4- oxo-2-(azaspiro[3.3]heptan-2- yl)-4H-pyrido[1,2-a]pyrimidin-9- yl)ethyl)amino)benzoic acid |
| 11-324 | 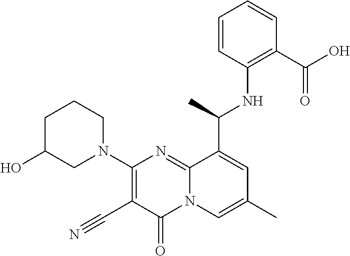 [SMILES: Cc1cc([C@@H](C)Nc2ccccc2C(=O)O)c2nc(N3CCCC(O)C3)c(C#N)c(=O)n2c1] | 2-(((1R)-1-(3-cyano-2-(3- hydroxypiperidin-1-yl)-7-methyl- 4-oxo-4H-pyrido[1,2-a]pyrimidin-9- yl)ethyl)amino)benzoic acid |
| 11-325 | 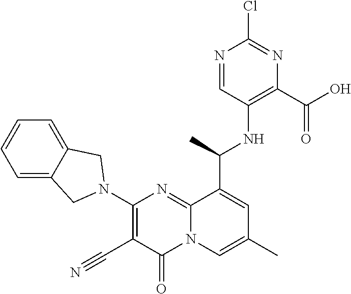 [SMILES: Cc1cc([C@@H](C)Nc2cnc(Cl)nc2C(=O)O)c2nc(N3Cc4ccccc4C3)c(C#N)c(=O)n2c1] | (R)-2-chloro-5-((1-(3-cyano-2- (isoindolin-2-yl)-7-methyl-4-oxo- 4H-pyrido[1,2-a]pyrimidin-9- yl)ethyl)amino)pyrimidine-4- carboxylic acid |
| 11-326 | 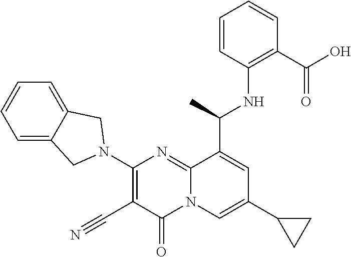 [SMILES: C[C@@H](Nc1ccccc1C(=O)O)c1cc(C2CC2)cn2c(=O)c(C#N)c(N3Cc4ccccc4C3)nc12] | (R)-2-((1-(3-cyano-7-cyclopropyl- 2-(isoindolin-2-yl)-4-oxo- 4H-pyrido[1,2-a]pyrimidin-9- yl)ethyl)amino)benzoic acid |
| 11-327 | 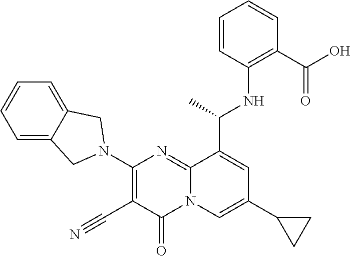 [SMILES: C[C@H](Nc1ccccc1C(=O)O)c1cc(C2CC2)cn2c(=O)c(C#N)c(N3Cc4ccccc4C3)nc12] | (S)-2-((1-(3-cyano-7-cyclopropyl- 2-(isoindolin-2-yl)-4-oxo- 4H-pyrido[1,2-a]pyrimidin-9- yl)ethyl)amino)benzoic acid |
| 11-328 | 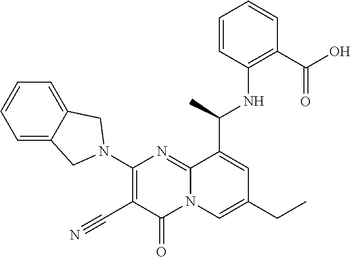 [SMILES: CCc1cc([C@@H](C)Nc2ccccc2C(=O)O)c2nc(N3Cc4ccccc4C3)c(C#N)c(=O)n2c1] | (R)-2-((1-(3-cyano-7-ethyl-2- (isoindolin-2-yl)-4-oxo- 4H-pyrido[1,2-a]pyrimidin-9- yl)ethyl)amino)benzoic acid |
| 11-329 | 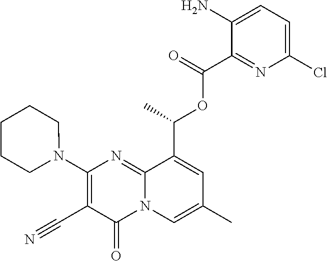 [SMILES: Cc1cc([C@H](C)OC(=O)c2nc(Cl)ccc2N)c2nc(N3CCCCC3)c(C#N)c(=O)n2c1] | (S)-1-(3-cyano-7-methyl-4-oxo-2- (piperidin-1-yl)-4H-pyrido[1,2- a]pyrimidin-9-yl)ethyl 3-amino-6- chloropicolinate |
| 11-330 | 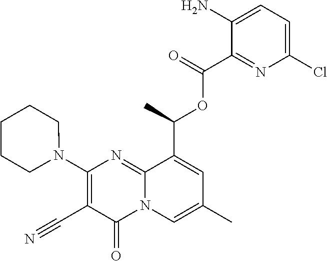 [SMILES: Cc1cc([C@@H](C)OC(=O)c2nc(Cl)ccc2N)c2nc(N3CCCCC3)c(C#N)c(=O)n2c1] | (R)-1-(3-cyano-7-methyl-4-oxo- 2-(piperidin-1-yl)-4H-pyrido[1,2- a]pyrimidin-9-yl)ethyl 3-amino-6- chloropicolinate |
| 11-331 | 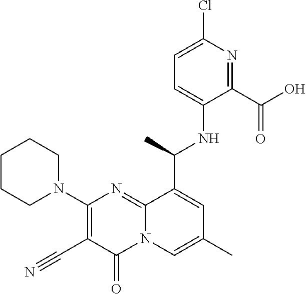 [SMILES: Cc1cc([C@@H](C)Nc2ccc(Cl)nc2C(=O)O)c2nc(N3CCCCC3)c(C#N)c(=O)n2c1] | (R)-6-chloro-3-((1-(3-cyano-7- methyl-4-oxo-2-(piperidin-1-yl)- 4H-pyrido[1,2-a]pyrimidin-9- yl)ethyl)amino)picolinic acid |
| 11-332 | 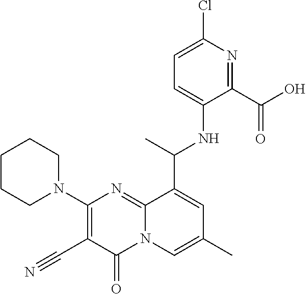 [SMILES: Cc1cc(C(C)Nc2ccc(Cl)nc2C(=O)O)c2nc(N3CCCCC3)c(C#N)c(=O)n2c1] | 6-chloro-3-((1-(3-cyano-7- methyl-4-oxo-2-(piperidin-1-yl)- 4H-pyrido[1,2-a]pyrimidin-9- yl)ethyl)amino)picolinic acid |
| 11-333 | 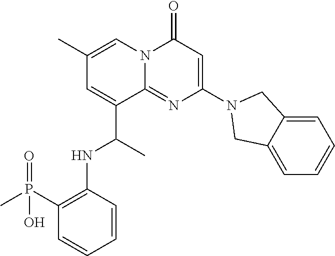 [SMILES: Cc1cc(C(C)Nc2ccccc2P(C)(=O)O)c2nc(N3Cc4ccccc4C3)cc(=O)n2c1] | (2-((1-(2-(isoindolin-2-yl)-7- methyl-4-oxo-4H-pyrido[1,2- a]pyrimidin-9- yl)ethyl)amino)phenyl)(methyl) phosphinic acid |
| 11-334 | 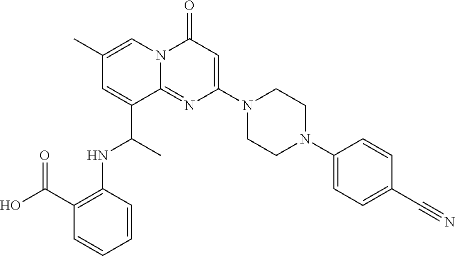 [SMILES: Cc1cc(C(C)Nc2ccccc2C(=O)O)c2nc(N3CCN(c4ccc(C#N)cc4)CC3)cc(=O)n2c1] | 2-((1-(2-(4-(4- cyanophenyl)piperazin-1-yl)-7- methyl-4-oxo- 4H-pyrido[1,2-a]pyrimidin-9- yl)ethyl)amino)benzoic acid |
| 11-335 | 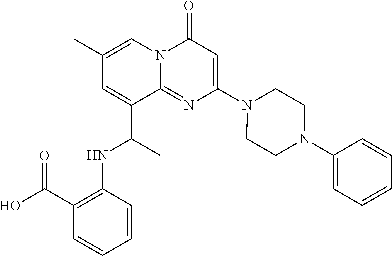 [SMILES: Cc1cc(C(C)Nc2ccccc2C(=O)O)c2nc(N3CCN(c4ccccc4)CC3)cc(=O)n2c1] | 2-((1-(7-methyl-4-oxo-2-(4- phenylpiperazin-1-yl)- 4H-pyrido[1,2-a]pyrimidin-9- yl)ethyl)amino)benzoic acid |
| 11-336 | 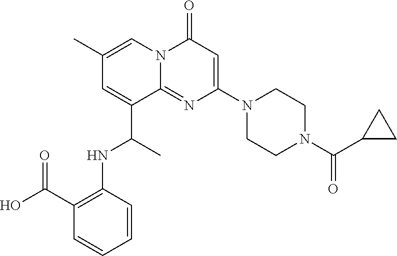 [SMILES: Cc1cc(C(C)Nc2ccccc2C(=O)O)c2nc(N3CCN(C(=O)C4CC4)CC3)cc(=O)n2c1] | 2-((1-(2-(4- (cyclopropanecarbonyl)piperazin- 1-yl)-7-methyl-4-oxo- 4H-pyrido[1,2-a]pyrimidin-9- yl)ethyl)amino)benzoic acid |
| 11-337 | 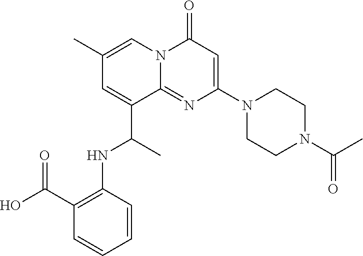 [SMILES: CC(=O)N1CCN(c2cc(=O)n3cc(C)cc(C(C)Nc4ccccc4C(=O)O)c3n2)CC1] | 2-((1-(2-(4-acetylpiperazin-1-yl)- 7-methyl-4-oxo- 4H-pyrido[1,2-a]pyrimidin-9- yl)ethyl)amino)benzoic acid |
| 11-338 | 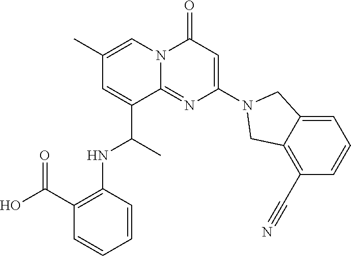 [SMILES: Cc1cc(C(C)Nc2ccccc2C(=O)O)c2nc(N3Cc4cccc(C#N)c4C3)cc(=O)n2c1] | 2-((1-(2-(4-cyanoisoindolin-2-yl)- 7-methyl-4-oxo- 4H-pyrido[1,2-a]pyrimidin-9- yl)ethyl)amino)benzoic acid |
| 11-339 | 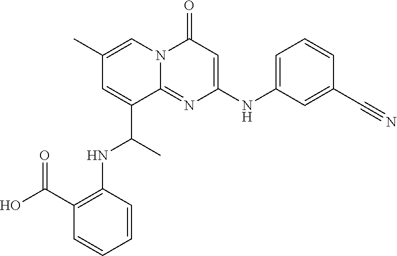 [SMILES: Cc1cc(C(C)Nc2ccccc2C(=O)O)c2nc(Nc3cccc(C#N)c3)cc(=O)n2c1] | 2-((1-(2-((3-cyanophenyl)amino)- 7-methyl-4-oxo- 4H-pyrido[1,2-a]pyrimidin-9- yl)ethyl)amino)benzoic acid |
| 11-340 | 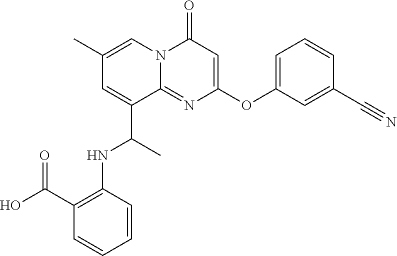 [SMILES: Cc1cc(C(C)Nc2ccccc2C(=O)O)c2nc(Oc3cccc(C#N)c3)cc(=O)n2c1] | 2-((1-(2-(3-cyanophenoxy)-7- methyl-4-oxo- 4H-pyrido[1,2-a]pyrimidin-9- yl)ethyl)amino)benzoic acid |
| 11-341 | 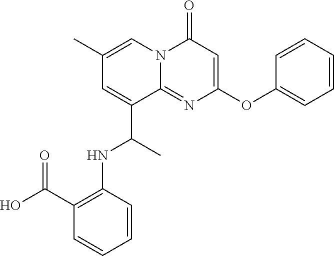 [SMILES: Cc1cc(C(C)Nc2ccccc2C(=O)O)c2nc(Oc3ccccc3)cc(=O)n2c1] | 2-((1-(7-methyl-4-oxo-2- phenoxy-4H-pyrido[1,2- a]pyrimidin-9- yl)ethyl)amino)benzoic acid |
| 11-342 | 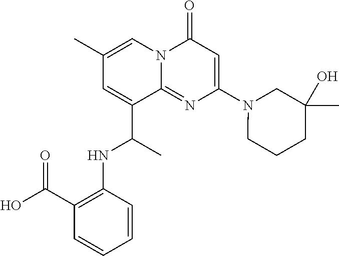 [SMILES: Cc1cc(C(C)Nc2ccccc2C(=O)O)c2nc(N3CCCC(C)(O)C3)cc(=O)n2c1] | 2-((1-(2-(3-hydroxy-3- methylpiperidin-1-yl)-7-methyl-4- oxo-4H-pyrido[1,2-a]pyrimidin-9- yl)ethyl)amino)benzoic acid |
| 11-343 | 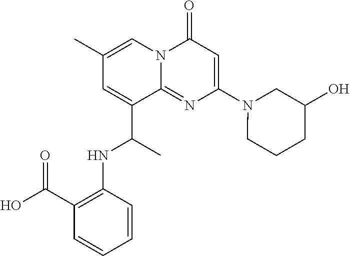 [SMILES: Cc1cc(C(C)Nc2ccccc2C(=O)O)c2nc(N3CCCC(O)C3)cc(=O)n2c1] | 2-((1-(2-(3-hydroxypiperidin-1- yl)-7-methyl-4-oxo- 4H-pyrido[1,2-a]pyrimidin-9- yl)ethyl)amino)benzoic acid |
| 11-344 | 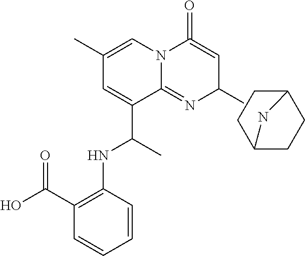 | 2-((1-(2-(7- azabicyclo[2.2.1]heptan-7-yl)-7- methyl-4-oxo- 4H-pyrido[1,2-a]pyrimidin-9- yl)ethyl)amino)benzoic acid |
| 11-345 | 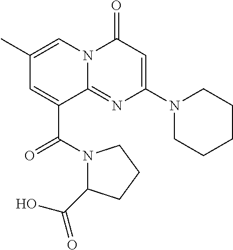 [SMILES: Cc1cc(C(=O)N2CCCC2C(=O)O)c2nc(N3CCCCC3)cc(=O)n2c1] | (7-methyl-4-oxo-2-(piperidin-1- yl)-4H-pyrido[1,2-a]pyrimidin-9- carbonyl)proline |
| 11-346 | 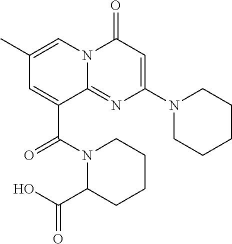 [SMILES: Cc1cc(C(=O)N2CCCCC2C(=O)O)c2nc(N3CCCCC3)cc(=O)n2c1] | 1-(7-methyl-4-oxo-2-(piperidin-1- yl)-4H-pyrido[1,2-a]pyrimidine-9- carbonyl)piperidine-2-carboxylic acid |
| 11-347 | 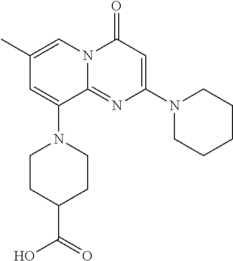 [SMILES: Cc1cc(N2CCC(C(=O)O)CC2)c2nc(N3CCCCC3)cc(=O)n2c1] | 1-(7-methyl-4-oxo-2-(piperidin-1- yl)-4H-pyrido[1,2-a]pyrimidin-9- yl)piperidine-4-carboxylic acid |
| 11-348 | 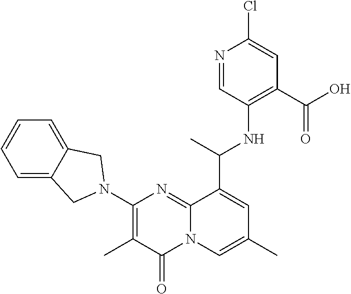 [SMILES: Cc1cc(C(C)Nc2cnc(Cl)cc2C(=O)O)c2nc(N3Cc4ccccc4C3)c(C)c(=O)n2c1] | 2-chloro-5-((1-(2-(isoindolin-2- yl)-3,7-dimethyl-4-oxo-4H- pyrido[1,2-a]pyrimidin-9- yl)ethyl)amino)isonicotinic acid |
| 11-349 | 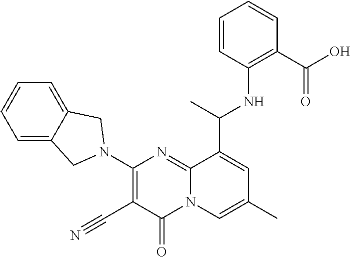 [SMILES: Cc1cc(C(C)Nc2ccccc2C(=O)O)c2nc(N3Cc4ccccc4C3)c(C#N)c(=O)n2c1] | 2-((1-(3-cyano-2-(isoindolin-2-yl)- 7-methyl-4-oxo- 4H-pyrido[1,2-a]pyrimidin-9- yl)ethyl)amino)benzoic acid |
| 11-350 | 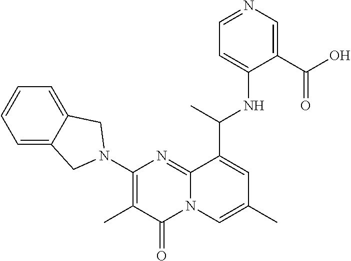 [SMILES: Cc1cc(C(C)Nc2ccncc2C(=O)O)c2nc(N3Cc4ccccc4C3)c(C)c(=O)n2c1] | 4-((1-(2-(isoindolin-2-yl)-3,7- dimethyl-4-oxo-4H-pyrido[1,2- a]pyrimidin-9- yl)ethyl)amino)nicotinic acid |
| 11-351 | 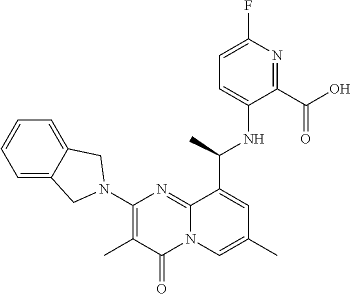 [SMILES: Cc1cc([C@@H](C)Nc2ccc(F)nc2C(=O)O)c2nc(N3Cc4ccccc4C3)c(C)c(=O)n2c1] | (R)-6-fluoro-3-((1-(2-(isoindolin- 2-yl)-3,7-dimethyl-4-oxo- 4H-pyrido[1,2-a]pyrimidin-9- yl)ethyl)amino)picolinic acid |
| 11-352 | 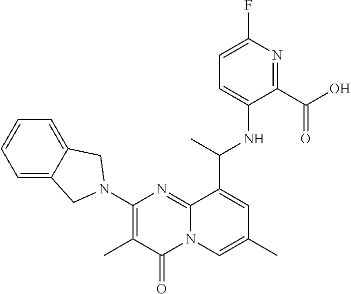 [SMILES: Cc1cc(C(C)Nc2ccc(F)nc2C(=O)O)c2nc(N3Cc4ccccc4C3)c(C)c(=O)n2c1] | 6-fluoro-3-((1-(2-(isoindolin-2-yl)- 3,7-dimethyl-4-oxo- 4H-pyrido[1,2-a]pyrimidin-9- yl)ethyl)amino)picolinic acid |
| 11-353 | 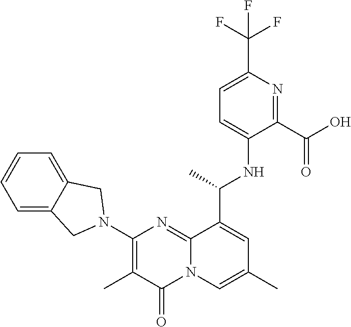 [SMILES: Cc1cc([C@H](C)Nc2ccc(C(F)(F)F)nc2C(=O)O)c2nc(N3Cc4ccccc4C3)c(C)c(=O)n2c1] | (S)-3-((1-(2-(isoindolin-2-yl)-3,7- dimethyl-4-oxo-4H-pyrido[1,2-a]pyrimidin-9- yl)ethyl)amino)-6-(trifluoromethyl) picolinic acid |
| 11-354 | 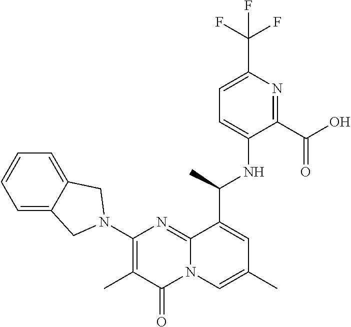 [SMILES: Cc1cc([C@@H](C)Nc2ccc(C(F)(F)F)nc2C(=O)O)c2nc(N3Cc4ccccc4C3)c(C)c(=O)n2c1] | (R)-3-((1-(2-(isoindolin-2-yl)-3,7- dimethyl-4-oxo-4H-pyrido[1,2-a]pyrimidin-9- yl)ethyl)amino)-6- (trifluoromethyl)picolinic acid |
| 11-355 | 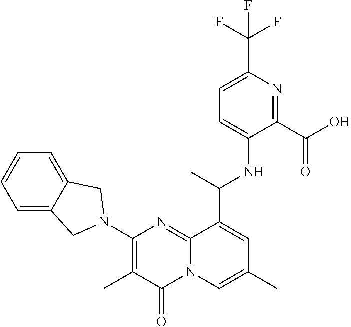 [SMILES: Cc1cc(C(C)Nc2ccc(C(F)(F)F)nc2C(=O)O)c2nc(N3Cc4ccccc4C3)c(C)c(=O)n2c1] | 3-((1-(2-(isoindolin-2-yl)-3,7- dimethyl-4-oxo-4H-pyrido[1,2-a]pyrimidin-9- yl)ethyl)amino)-6-(trifluoromethyl) picolinic acid |
| 11-356 | 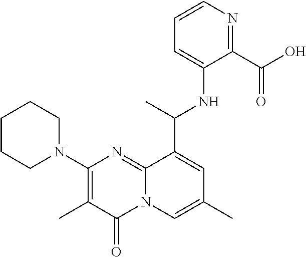 [SMILES: Cc1cc(C(C)Nc2cccnc2C(=O)O)c2nc(N3CCCCC3)c(C)c(=O)n2c1] | 3-((1-(3,7-dimethyl-4-oxo-2- (piperidin-1-yl)-4H-pyrido[1,2- a]pyrimidin-9- yl)ethyl)amino)picolinic acid |
| 11-357 | 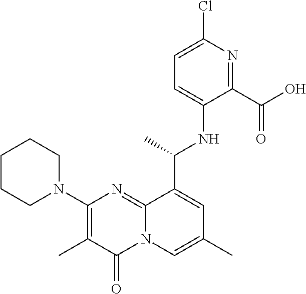 [SMILES: Cc1cc([C@H](C)Nc2ccc(Cl)nc2C(=O)O)c2nc(N3CCCCC3)c(C)c(=O)n2c1] | (S)-6-chloro-3-((1-(3,7-dimethyl- 4-oxo-2-(piperidin-1-yl)-4H- pyrido[1,2-a]pyrimidin-9- yl)ethyl)amino)picolinic acid |
| 11-358 | 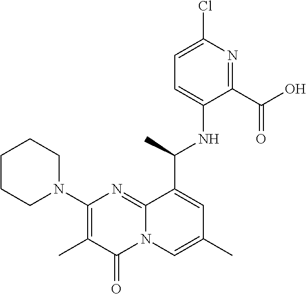 [SMILES: Cc1cc([C@@H](C)Nc2ccc(Cl)nc2C(=O)O)c2nc(N3CCCCC3)c(C)c(=O)n2c1] | (R)-6-chloro-3-((1-(3,7-dimethyl- 4-oxo-2-(piperidin-1-yl)- 4H-pyrido[1,2-a]pyrimidin-9- yl)ethyl)amino)picolinic acid |
| 11-359 | 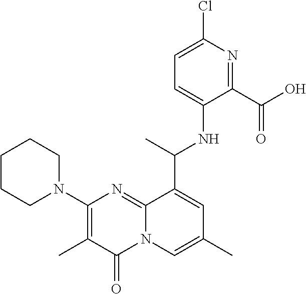 [SMILES: Cc1cc(C(C)Nc2ccc(Cl)nc2C(=O)O)c2nc(N3CCCCC3)c(C)c(=O)n2c1] | 6-chloro-3-((1-(3,7-dimethyl-4- oxo-2-(piperidin-1-yl)- 4H-pyrido[1,2-a]pyrimidin-9- yl)ethyl)amino)picolinic acid |
| 11-360 | 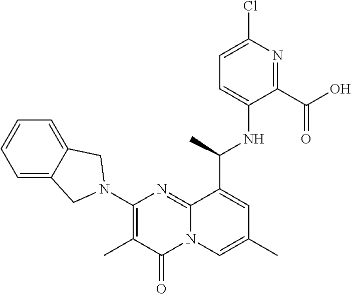 [SMILES: Cc1cc([C@@H](C)Nc2ccc(Cl)nc2C(=O)O)c2nc(N3Cc4ccccc4C3)c(C)c(=O)n2c1] | (R)-6-chloro-3-((1-(2-(isoindolin- 2-yl)-3,7-dimethyl-4-oxo- 4H-pyrido[1,2-a]pyrimidin-9- yl)ethyl)amino)picolinic acid |
| 11-361 | 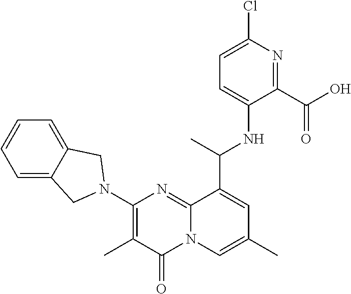 [SMILES: Cc1cc(C(C)Nc2ccc(Cl)nc2C(=O)O)c2nc(N3Cc4ccccc4C3)c(C)c(=O)n2c1] | 6-chloro-3-((1-(2-isoindolin-2- yl)-3,7-dimethyl-4-oxo- 4H-pyrido[1,2-a]pyrimidin-9- yl)ethyl)amino)picolinic acid |
| 11-362 | 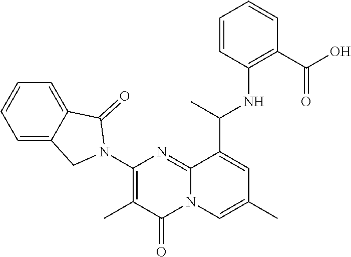 [SMILES: Cc1cc(C(C)Nc2ccccc2C(=O)O)c2nc(N3Cc4ccccc4C3=O)c(C)c(=O)n2c1] | 2-((1-(3,7-dimethyl-4-oxo-2-(1- oxoisoindolin-2-yl)- 4H-pyrido[1,2-a]pyrimidin-9- yl)ethyl)amino)benzoic acid |
| 11-363 | 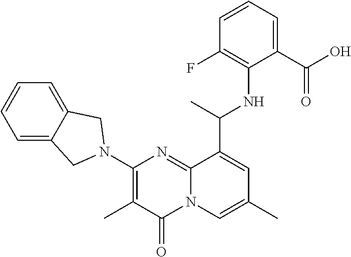 [SMILES: Cc1cc(C(C)Nc2c(F)cccc2C(=O)O)c2nc(N3Cc4ccccc4C3)c(C)c(=O)n2c1] | 3-fluoro-2-((1-(2-(isoindolin-2-yl)- 3,7-dimethyl-4-oxo- 4H-pyrido[1,2-a]pyrimidin-9- yl)ethyl)amino)benzoic acid |
| 11-364 | 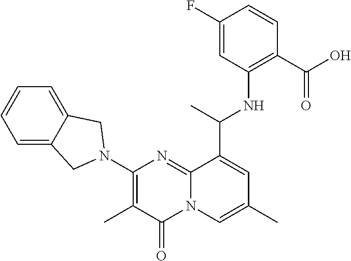 [SMILES: Cc1cc(C(C)Nc2cc(F)ccc2C(=O)O)c2nc(N3Cc4ccccc4C3)c(C)c(=O)n2c1] | 4-fluoro-2-((1-(2-(isoindolin-2-yl)- 3,7-dimethyl-4-oxo- 4H-pyrido[1,2-a]pyrimidin-9- yl)ethyl)amino)benzoic acid |
| 11-365 | 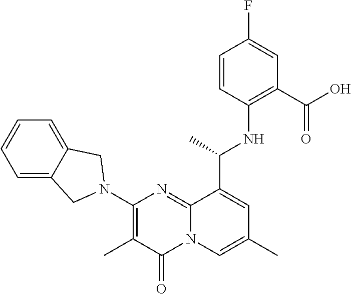 [SMILES: Cc1cc([C@H](C)Nc2ccc(F)cc2C(=O)O)c2nc(N3Cc4ccccc4C3)c(C)c(=O)n2c1] | (S)-5-fluoro-2-((1-(2-(isoindolin- 2-yl)-3,7-dimethyl-4-oxo- 4H-pyrido[1,2-a]pyrimidin-9- yl)ethyl)amino)benzoic acid |
| 11-366 | 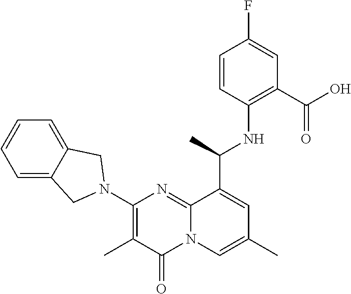 [SMILES: Cc1cc([C@@H](C)Nc2ccc(F)cc2C(=O)O)c2nc(N3Cc4ccccc4C3)c(C)c(=O)n2c1] | (R)-5-fluoro-2-((1-(2-(isoindolin- 2-yl)-3,7-dimethyl-4-oxo- 4H-pyrido[1,2-a]pyrimidin-9- yl)ethyl)amino)benzoic acid |
| 11-367 | 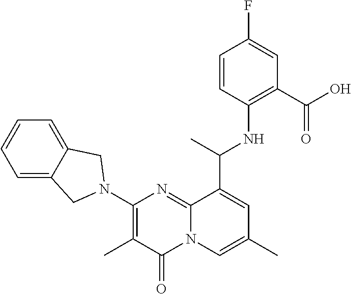 [SMILES: Cc1cc(C(C)Nc2ccc(F)cc2C(=O)O)c2nc(N3Cc4ccccc4C3)c(C)c(=O)n2c1] | 5-fluoro-2-((1-(2-(isoindolin-2-yl)- 3,7-dimethyl-4-oxo- 4H-pyrido[1,2-a]pyrimidin-9- yl)ethyl)amino)benzoic acid |
| 11-368 | 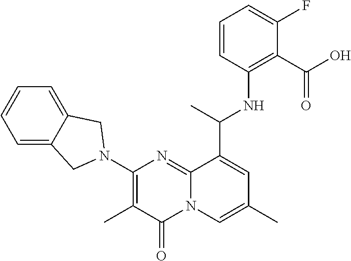 [SMILES: Cc1cc(C(C)Nc2cccc(F)c2C(=O)O)c2nc(N3Cc4ccccc4C3)c(C)c(=O)n2c1] | 2-fluoro-6-((1-(2-(isoindolin-2-yl)- 3,7-dimethyl-4-oxo- 4H-pyrido[1,2-a]pyrimidin-9- yl)ethyl)amino)benzoic acid |
| 11-370 | 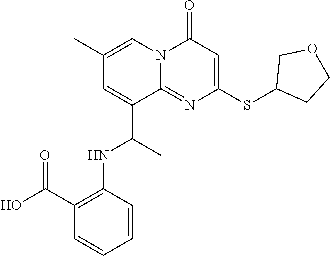 [SMILES: Cc1cc(C(C)Nc2ccccc2C(=O)O)c2nc(SC3CCOC3)cc(=O)n2c1] | 2-((1-(7-methyl-4-oxo-2- ((tetrahydrofuran-3-yl)thio)- 4H-pyrido[1,2-a]pyrimidin-9- yl)ethyl)amino)benzoic acid |
| 11-371 | 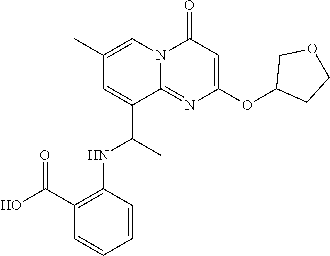 [SMILES: Cc1cc(C(C)Nc2ccccc2C(=O)O)c2nc(OC3CCOC3)cc(=O)n2c1] | 2-((1-(7-methyl-4-oxo-2- ((tetrahydrofuran-3-yl)oxy)- 4H-pyrido[1,2-a]pyrimidin-9- yl)ethyl)amino)benzoic acid |
| 11-372 | 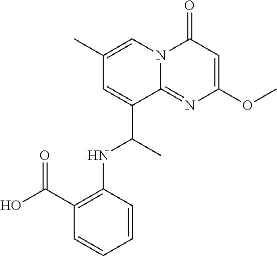 [SMILES: COc1cc(=O)n2cc(C)cc(C(C)Nc3ccccc3C(=O)O)c2n1] | 2-((1-(2-methoxy-7-methyl-4- oxo-4H-pyrido[1,2-a]pyrimidin-9- yl)ethyl)amino)benzoic acid |
| 11-373 | 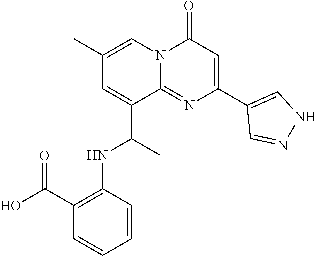 [SMILES: Cc1cc(C(C)Nc2ccccc2C(=O)O)c2nc(-c3cn[nH]c3)cc(=O)n2c1] | 2-((1-(7-methyl-4-oxo-2-(1H- pyrazol-4-yl)- 4H-pyrido[1,2-a]pyrimidin-9- yl)ethyl)amino)benzoic acid |
| 11-374 | 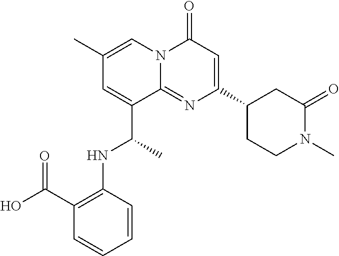 [SMILES: Cc1cc([C@H](C)Nc2ccccc2C(=O)O)c2nc([C@H]3CCN(C)C(=O)C3)cc(=O)n2c1] | 2-(((S)-1-(7-methyl-2-((S)-1- methyl-2-oxopiperidin-4-yl)-4- oxo-4H-pyrido[1,2-a]pyrimidin-9- yl)ethyl)amino)benzoic acid |
| 11-375 | 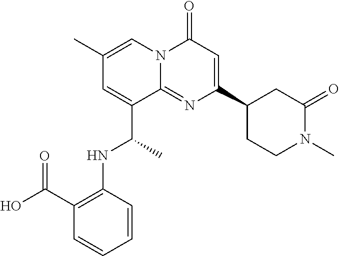 [SMILES: Cc1cc([C@H](C)Nc2ccccc2C(=O)O)c2nc([C@@H]3CCN(C)C(=O)C3)cc(=O)n2c1] | 2-(((S)-1-(7-methyl-2-((R)-1- methyl-2-oxopiperidin-4-yl)-4- oxo-4H-pyrido[1,2-a]pyrimidin-9- yl)ethyl)amino)benzoic acid |
| 11-376 | 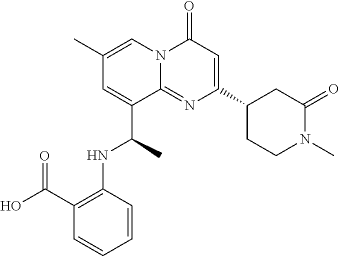 [SMILES: Cc1cc([C@@H](C)Nc2ccccc2C(=O)O)c2nc([C@H]3CCN(C)C(=O)C3)cc(=O)n2c1] | 2-(((R)-1-(7-methyl-2-((S)-1- methyl-2-oxopiperidin-4-yl)-4- oxo-4H-pyrido[1,2-a]pyrimidin-9- yl)ethyl)amino)benzoic acid |
| 11-377 | 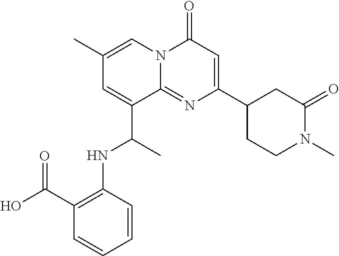 [SMILES: Cc1cc(C(C)Nc2ccccc2C(=O)O)c2nc(C3CCN(C)C(=O)C3)cc(=O)n2c1] | 2-((1-(7-methyl-2-(1-methyl-2- oxopiperidin-4-yl)-4-oxo- 4H-pyrido[1,2-a]pyrimidin-9- yl)ethyl)amino)benzoic acid |
| 11-378 | 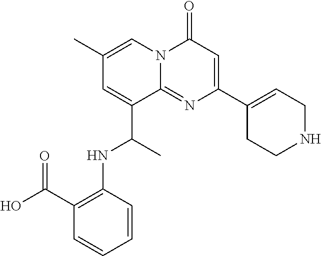 [SMILES: Cc1cc(C(C)Nc2ccccc2C(=O)O)c2nc(C3=CCNCC3)cc(=O)n2c1] | 2-((1-(7-methyl-4-oxo-2-(1,2,3,6- tetrahydropyridin-4-yl)- 4H-pyrido[1,2-a]pyrimidin-9- yl)ethyl)amino)benzoic acid |
| 11-379 | 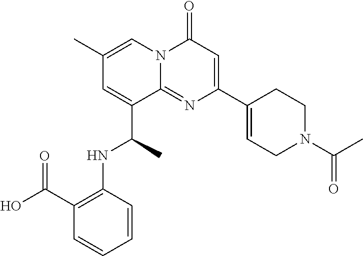 [SMILES: CC(=O)N1CC=C(c2cc(=O)n3cc(C)cc([C@@H](C)Nc4ccccc4C(=O)O)c3n2)CC1] | (R)-2-((1-(2-(1-acetyl-1,2,3,6- tetrahydropyridin-4-yl)-7-methyl- 4-oxo-4H-pyrido[1,2-a]pyrimidin-9- yl)ethyl)amino)benzoic acid |
| 11-380 | 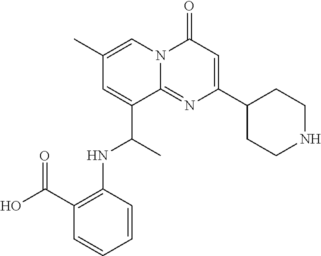 [SMILES: Cc1cc(C(C)Nc2ccccc2C(=O)O)c2nc(C3CCNCC3)cc(=O)n2c1] | 2-((1-(7-methyl-4-oxo-2- (piperidin-4-yl)- 4H-pyrido[1,2-a]pyrimidin-9- yl)ethyl)amino)benzoic acid |
| 11-381 | 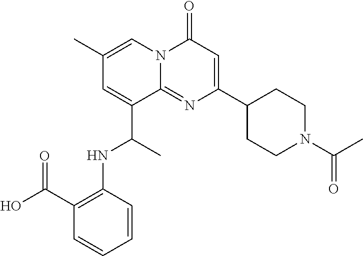 [SMILES: CC(=O)N1CCC(c2cc(=O)n3cc(C)cc(C(C)Nc4ccccc4C(=O)O)c3n2)CC1] | 2-((1-(2-(1-acetylpiperidin-4-yl)- 7-methyl-4-oxo- 4H-pyrido[1,2-a]pyrimidin-9- yl)ethyl)amino)benzoic acid |
| 11-382 | 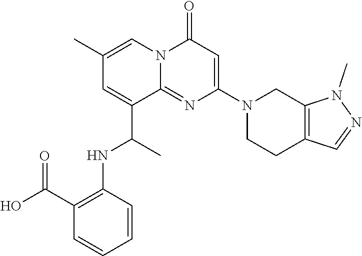 [SMILES: Cc1cc(C(C)Nc2ccccc2C(=O)O)c2nc(N3CCc4cnn(C)c4C3)cc(=O)n2c1] | 2-((1-(7-methyl-2-(1-methyl- 1,4,5,7-tetrahydro-6H- pyrazolo[3,4-c]pyridin-6-yl)-4- oxo-4H-pyrido[1,2-a]pyrimidin-9- yl)ethyl)amino)benzoic acid |
| 11-383 | 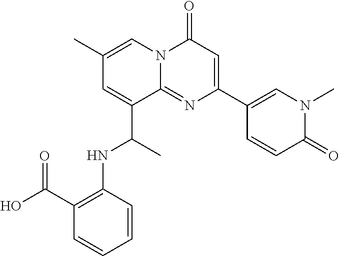 [SMILES: Cc1cc(C(C)Nc2ccccc2C(=O)O)c2nc(-c3ccc(=O)n(C)c3)cc(=O)n2c1] | 2-((1-(7-methyl-2-(1-methyl-6- oxo-1,6-dihydropyridin-3-yl)-4- oxo-4H-pyrido[1,2-a]pyrimidin-9- yl)ethyl)amino)benzoic acid |
| 11-384 | 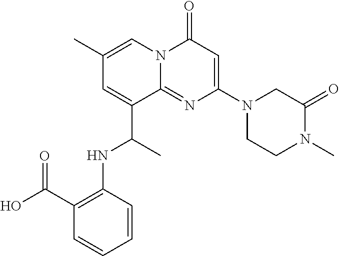 [SMILES: Cc1cc(C(C)Nc2ccccc2C(=O)O)c2nc(N3CCN(C)C(=O)C3)cc(=O)n2c1] | 2-((1-(7-methyl-2-(4-methyl-3- oxopiperazin-1-yl)-4-oxo- 4H-pyrido[1,2-a]pyrimidin-9- yl)ethyl)amino)benzoic acid |
| 11-385 | 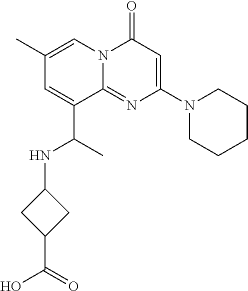 [SMILES: Cc1cc(C(C)NC2CC(C(=O)O)C2)c2nc(N3CCCCC3)cc(=O)n2c1] | 3-((1-(7-methyl-4-oxo-2- (piperidin-1-yl)-4H-pyrido[1,2- a]pyrimidin-9- yl)ethyl)amino)cyclobutane-1- carboxylic acid |
| 11-386 | 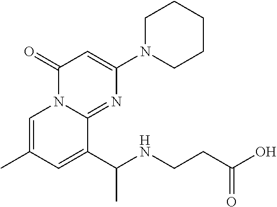 [SMILES: Cc1cc(C(C)NCCC(=O)O)c2nc(N3CCCCC3)cc(=O)n2c1] | 3-((1-(7-methyl-4-oxo-2- (piperidin-1-yl)-4H-pyrido[1,2- a]pyrimidin-9- yl)ethyl)propanoic acid |
| 11-387 | 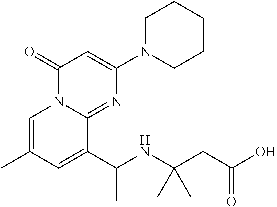 [SMILES: Cc1cc(C(C)NC(C)(C)CC(=O)O)c2nc(N3CCCCC3)cc(=O)n2c1] | 3-methyl-3-((1-(7-methyl-4-oxo- 2-(piperidin-1-yl)-4H-pyrido[1,2- a]pyrimidin-9- yl)ethyl)amino)butanoic acid |
| 11-388 | 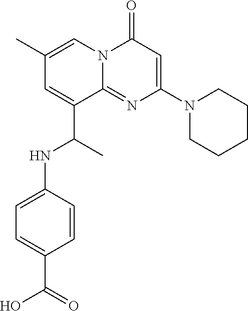 [SMILES: Cc1cc(C(C)Nc2ccc(C(=O)O)cc2)c2nc(N3CCCCC3)cc(=O)n2c1] | 4-((1-(7-methyl-4-oxo-2- (piperidin-1-yl)-4H-pyrido [1,2-a]pyrimidin-9- yl)ethyl)amino)benzoic acid |
| 11-389 | 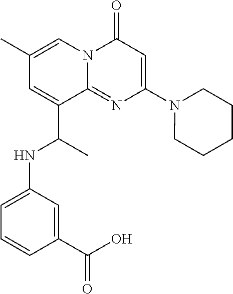 [SMILES: Cc1cc(C(C)Nc2cccc(C(=O)O)c2)c2nc(N3CCCCC3)cc(=O)n2c1] | 3-((1-(7-methyl-4-oxo-2- (piperidin-1-yl)- 4H-pyrido[1,2-a]pyrimidin-9- yl)ethyl)amino)benzoic acid |
| 11-390 | 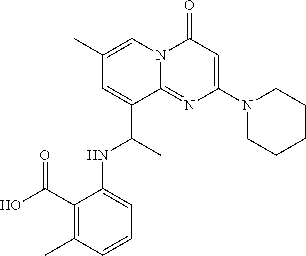 [SMILES: Cc1cc(C(C)Nc2cccc(C)c2C(=O)O)c2nc(N3CCCCC3)cc(=O)n2c1] | 2-methyl-6-((1-(7-methyl-4-oxo- 2-(piperidin-1-yl)- 4H-pyrido[1,2-a]pyrimidin-9- yl)ethyl)amino)benzoic acid |
| 11-391 | 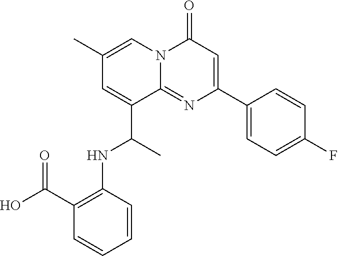 [SMILES: Cc1cc(C(C)Nc2ccccc2C(=O)O)c2nc(-c3ccc(F)cc3)cc(=O)n2c1] | 2-((1-(2-(4-fluorophenyl)-7- methyl-4-oxo- 4H-pyrido[1,2-a]pyrimidin-9- yl)ethyl)amino)benzoic acid |
| 11-392 | 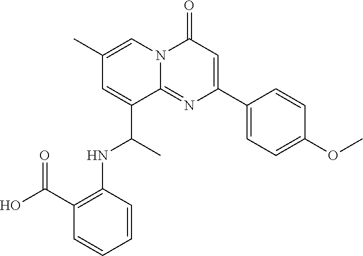 [SMILES: COc1ccc(-c2cc(=O)n3cc(C)cc(C(C)Nc4ccccc4C(=O)O)c3n2)cc1] | 2-((1-(2-(4-methoxyphenyl)-7- methyl-4-oxo- 4H-pyrido[1,2-a]pyrimidin-9- yl)ethyl)amino)benzoic acid |
| 11-393 | 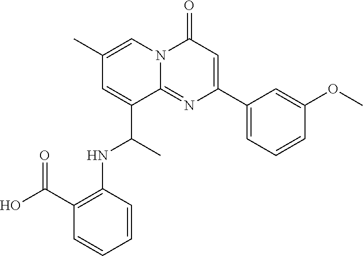 [SMILES: COc1cccc(-c2cc(=O)n3cc(C)cc(C(C)Nc4ccccc4C(=O)O)c3n2)c1] | 2-((1-(2-(3-methoxyphenyl)-7- methyl-4-oxo-4H-pyrido[1,2- a]pyrimidin-9- yl)ethyl)amino)benzoic acid |
| 11-394 | 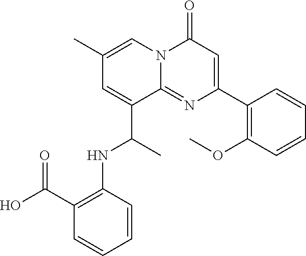 [SMILES: COc1ccccc1-c1cc(=O)n2cc(C)cc(C(C)Nc3ccccc3C(=O)O)c2n1] | 2-((1-(2-(2-methoxyphenyl)-7- methyl-4-oxo- 4H-pyrido[1,2-a]pyrimidin-9- yl)ethyl)amino)benzoic acid |
| 11-395 | 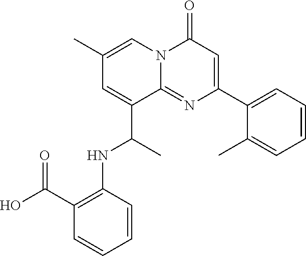 [SMILES: Cc1cc(C(C)Nc2ccccc2C(=O)O)c2nc(-c3ccccc3C)cc(=O)n2c1] | 2-((1-(7-methyl-4-oxo-2-(o-tolyl)- 4H-pyrido[1,2-a]pyrimidin-9- yl)ethyl)amino)benzoic acid |
| 11-396 | 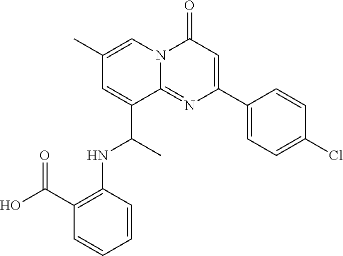 [SMILES: Cc1cc(C(C)Nc2ccccc2C(=O)O)c2nc(-c3ccc(Cl)cc3)cc(=O)n2c1] | 2-((1-(2-(4-chlorophenyl)-7- methyl-4-oxo- 4H-pyrido[1,2-a]pyrimidin-9- yl)ethyl)amino)benzoic acid |
| 11-397 | 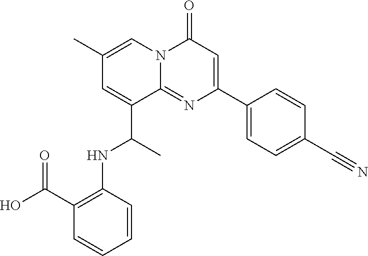 [SMILES: Cc1cc(C(C)Nc2ccccc2C(=O)O)c2nc(-c3ccc(C#N)cc3)cc(=O)n2c1] | 2-((1-(2-(4-cyanophenyl)-7- methyl-4-oxo- 4H-pyrido[1,2-a]pyrimidin-9- yl)ethyl)amino)benzoic acid |
| 11-398 | 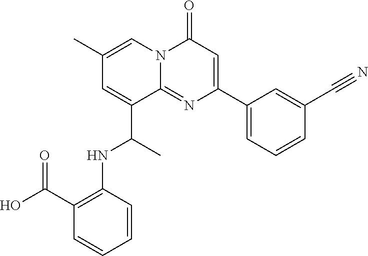 [SMILES: Cc1cc(C(C)Nc2ccccc2C(=O)O)c2nc(-c3cccc(C#N)c3)cc(=O)n2c1] | 2-((1-(2-(3-cyanophenyl)-7- methyl-4-oxo- 4H-pyrido[1,2-a]pyrimidin-9- yl)ethyl)amino)benzoic acid |
| 11-399 | 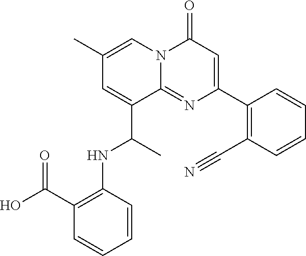 [SMILES: Cc1cc(C(C)Nc2ccccc2C(=O)O)c2nc(-c3ccccc3C#N)cc(=O)n2c1] | 2-((1-(2-(2-cyanophenyl)-7- methyl-4-oxo- 4H-pyrido[1,2-a]pyrimidin-9- yl)ethyl)amino)benzoic acid |
| 11-400 | 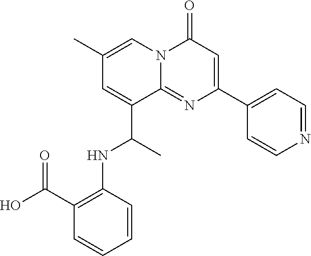 [SMILES: Cc1cc(C(C)Nc2ccccc2C(=O)O)c2nc(-c3ccncc3)cc(=O)n2c1] | 2-((1-(7-methyl-4-oxo-2-(pyridin- 4-yl)-4H-pyrido[1,2-a]pyrimidin-9- yl)ethyl)amino)benzoic acid |
| 11-401 | 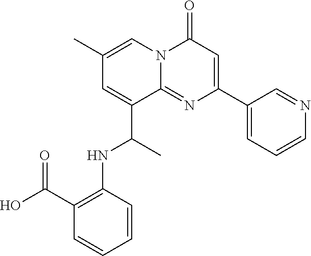 [SMILES: Cc1cc(C(C)Nc2ccccc2C(=O)O)c2nc(-c3cccnc3)cc(=O)n2c1] | 2-((1-(7-methyl-4-oxo-2-(pyridin- 3-yl)-4H-pyrido[1,2-a]pyrimidin-9- yl)ethyl)amino)benzoic acid |
| 11-402 | 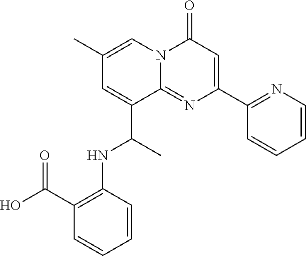 [SMILES: Cc1cc(C(C)Nc2ccccc2C(=O)O)c2nc(-c3ccccn3)cc(=O)n2c1] | 2-((1-(7-methyl-4-oxo-2-(pyridin- 2-yl)-4H-pyrido[1,2-a]pyrimidin-9- yl)ethyl)amino)benzoic acid |
| 11-403 | 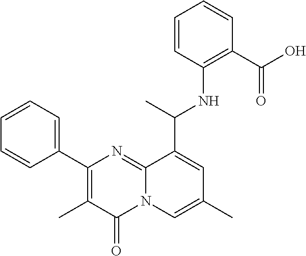 [SMILES: Cc1cc(C(C)Nc2ccccc2C(=O)O)c2nc(-c3ccccc3)c(C)c(=O)n2c1] | 2-((1-(3,7-dimethyl-4-oxo-2- phenyl-4H-pyrido[1,2-a]pyrimidin-9- yl)ethyl)amino)benzoic acid |
| 11-404 | 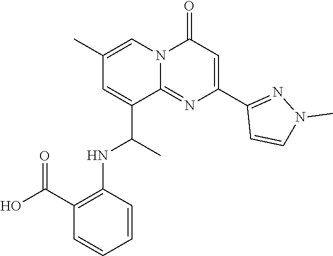 [SMILES: Cc1cc(C(C)Nc2ccccc2C(=O)O)c2nc(-c3ccn(C)n3)cc(=O)n2c1] | 2-((1-(7-methyl-2-(1-methyl-1H- pyrazol-3-yl)-4-oxo- 4H-pyrido[1,2-a]pyrimidin-9- yl)ethyl)amino)benzoic acid |
| 11-405 | 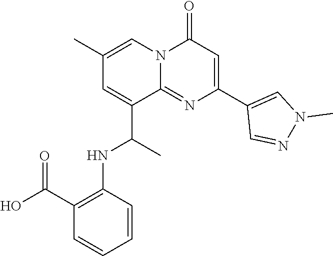 [SMILES: Cc1cc(C(C)Nc2ccccc2C(=O)O)c2nc(-c3cnn(C)c3)cc(=O)n2c1] | 2-((1-(7-methyl-2-(1-methyl-1H- pyrazol-4-yl)-4-oxo- 4H-pyrido[1,2-a]pyrimidin-9- yl)ethyl)amino)benzoic acid |
| 11-406 | 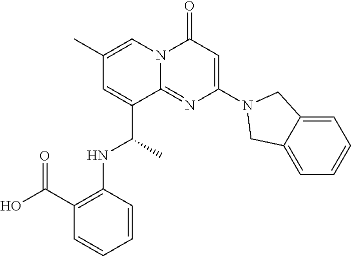 [SMILES: Cc1cc([C@H](C)Nc2ccccc2C(=O)O)c2nc(N3Cc4ccccc4C3)cc(=O)n2c1] | (S)-2-((1-(2-(isoindolin-2-yl)-7- methyl-4-oxo- 4H-pyrido[1,2-a]pyrimidin-9- yl)ethyl)amino)benzoic acid |
| 11-407 | 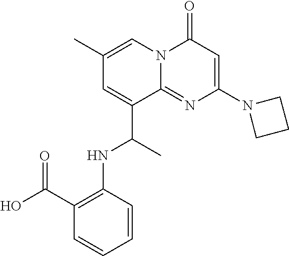 [SMILES: Cc1cc(C(C)Nc2ccccc2C(=O)O)c2nc(N3CCC3)cc(=O)n2c1] | 2-((1-(2-(azetidin-1-yl)-7-methyl- 4-oxo-4H-pyrido[1,2-a]pyrimidin-9- yl)ethyl)amino)benzoic acid |
| 11-408 | 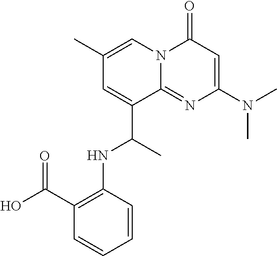 [SMILES: Cc1cc(C(C)Nc2ccccc2C(=O)O)c2nc(N(C)C)cc(=O)n2c1] | 2-((1-(2-(dimethylamino)-7- methyl-4-oxo- 4H-pyrido[1,2-a]pyrimidin-9- yl)ethyl)amino)benzoic acid |
| 11-409 | 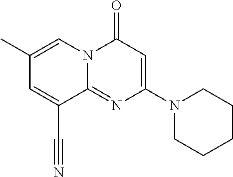 [SMILES: Cc1cc(C#N)c2nc(N3CCCCC3)cc(=O)n2c1] | 7-methyl-4-oxo-2-(piperidin-1-yl)- 4H-pyrido[1,2-a]pyrimidine-9- carbonitrile |
| 11-410 | 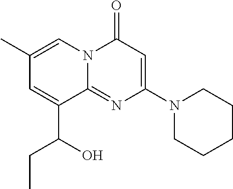 [SMILES: CCC(O)c1cc(C)cn2c(=O)cc(N3CCCCC3)nc12] | 9-(1-hydroxypropyl)-7-methyl-2- (piperidin-1-yl)-4H-pyrido[1,2- a]pyrimidin-4-one |
| 11-411 | 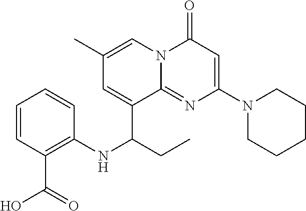 [SMILES: CCC(Nc1ccccc1C(=O)O)c1cc(C)cn2c(=O)cc(N3CCCCC3)nc12] | 2-((1-(7-methyl-4-oxo-2- (piperidin-1-yl)- 4H-pyrido[1,2-a]pyrimidin-9- yl)propyl)amino)benzoic acid |
| 11-412 | 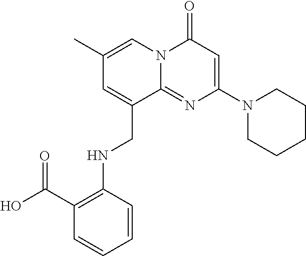 [SMILES: Cc1cc(CNc2ccccc2C(=O)O)c2nc(N3CCCCC3)cc(=O)n2c1] | 2-(((7-methyl-4-oxo-2-(piperidin- 1-yl)-4H-pyrido[1,2-a]pyrimidin-9- yl)ethyl)amino)benzoic acid |
| 11-413 | 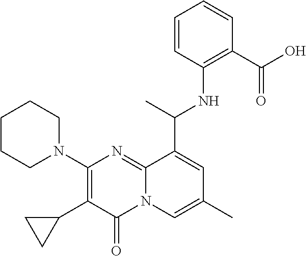 [SMILES: Cc1cc(C(C)Nc2ccccc2C(=O)O)c2nc(N3CCCCC3)c(C3CC3)c(=O)n2c1] | 2-((1-(3-cyclopropyl-7-methyl-4- oxo-2-(piperidin-1-yl)- 4H-pyrido[1,2-a]pyrimidin-9- yl)ethyl)amino)benzoic acid |
| 11-414 | 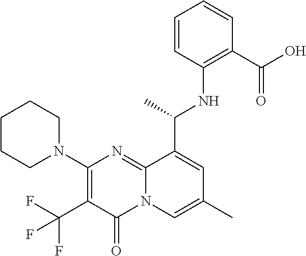 [SMILES: Cc1cc([C@H](C)Nc2ccccc2C(=O)O)c2nc(N3CCCCC3)c(C(F)(F)F)c(=O)n2c1] | (S)-2-((1-(7-methyl-4-oxo-2- (piperidin-1-yl)-3- (trifluoromethyl)- 4H-pyrido[1,2-a]pyrimidin-9- yl)ethyl)amino)benzoic acid |
| 11-415 | 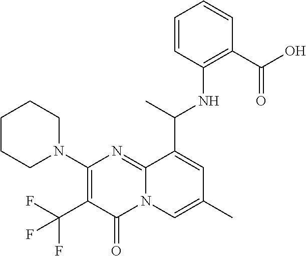 [SMILES: Cc1cc(C(C)Nc2ccccc2C(=O)O)c2nc(N3CCCCC3)c(C(F)(F)F)c(=O)n2c1] | 2-((1-(7-methyl-4-oxo-2- (piperidin-1-yl)-3- (trifluoromethyl)- 4H-pyrido[1,2-a]pyrimidin-9- yl)ethyl)amino)benzoic acid |
| 11-416 | 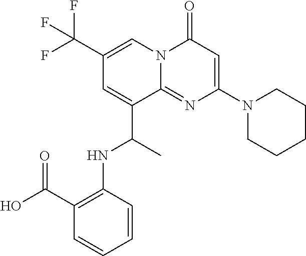 [SMILES: CC(Nc1ccccc1C(=O)O)c1cc(C(F)(F)F)cn2c(=O)cc(N3CCCCC3)nc12] | 2-((1-(4-oxo-2-(piperidin-1-yl)-7- (trifluoromethyl)- 4H-pyrido[1,2-a]pyrimidin-9- yl)ethyl)amino)benzoic acid |
| 11-417 | 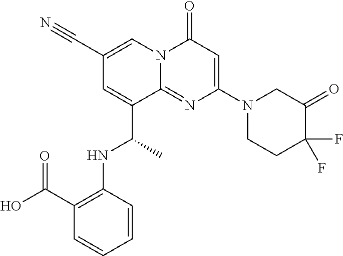 [SMILES: C[C@H](Nc1ccccc1C(=O)O)c1cc(C#N)cn2c(=O)cc(N3CCC(F)(F)C(=O)C3)nc12] | (S)-2-((1-(7-cyano-2-(4,4- difluoropiperidin-1-yl)-4-oxo- 4H-pyrido[1,2-a]pyrimidin-9- yl)ethyl)amino)benzoic acid |
| 11-418 | 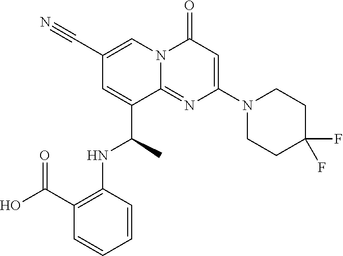 [SMILES: C[C@@H](Nc1ccccc1C(=O)O)c1cc(C#N)cn2c(=O)cc(N3CCC(F)(F)CC3)nc12] | (R)-2-((1-(7-cyano-2-(4,4- difluoropiperidin-1-yl)-4-oxo- 4H-pyrido[1,2-a]pyrimidin-9- yl)ethyl)amino)benzoic acid |
| 11-419 | 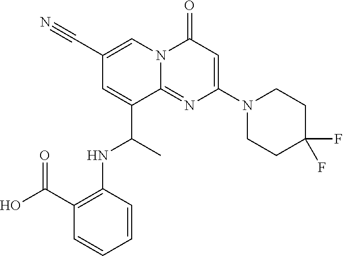 [SMILES: CC(Nc1ccccc1C(=O)O)c1cc(C#N)cn2c(=O)cc(N3CCC(F)(F)CC3)nc12] | 2-((1-(7-cyano-2-(4,4- difluoropiperidin-1-yl)-4-oxo- 4H-pyrido[1,2-a]pyrimidin-9- yl)ethyl)amino)benzoic acid |
| 11-420 | 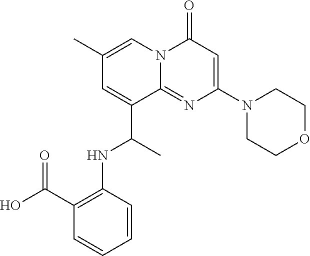 [SMILES: Cc1cc(C(C)Nc2ccccc2C(=O)O)c2nc(N3CCOCC3)cc(=O)n2c1] | 2-((1-(7-methyl-2-morpholino-4- oxo-4H-pyrido[1,2-a]pyrimidin-9- yl)ethyl)amino)benzoic acid |
| 11-421 | 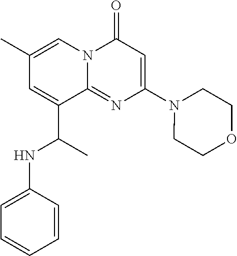 [SMILES: Cc1cc(C(C)Nc2ccccc2)c2nc(N3CCOCC3)cc(=O)n2c1] | 7-methyl-2-morpholino-9-(1- (phenylamino)ethyl)-4H- pyrido[1,2-a]pyrimidin-4-one |
| 11-422 | 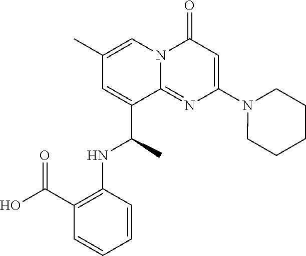 [SMILES: Cc1cc([C@@H](C)Nc2ccccc2C(=O)O)c2nc(N3CCCCC3)cc(=O)n2c1] | (R)-2-((1-(4-oxo-2-(piperidin-1- yl)-4H-pyrido[1,2-a]pyrimidin-9- yl)ethyl)amino)benzoic acid |
| 11-423 | 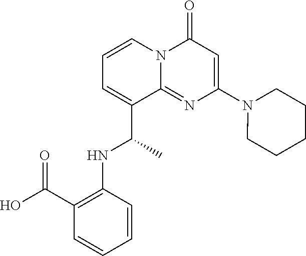 [SMILES: C[C@H](Nc1ccccc1C(=O)O)c1cccn2c(=O)cc(N3CCCCC3)nc12] | (S)-2-((1-(4-oxo-2-(piperidin-1- yl)-4H-pyrido[1,2-a]pyrimidin-9- yl)ethyl)amino)benzoic acid |
| 11-424 | 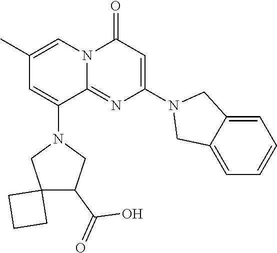 [SMILES: Cc1cc(N2CC(C(=O)O)C3(CCC3)C2)c2nc(N3Cc4ccccc4C3)cc(=O)n2c1] | 6-(2-(isoindolin-2-yl)-7-methyl-4- oxo-4H-pyrido[1,2-a]pyrimidin-9- yl)-6-azaspiro[3.4]octane-8- carboxylic acid |
| 11-425 | 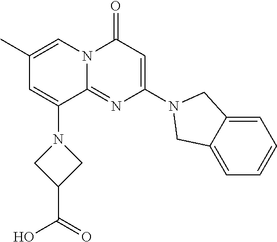 [SMILES: Cc1cc(N2CC(C(=O)O)C2)c2nc(N3Cc4ccccc4C3)cc(=O)n2c1] | 1-(2-(isoindolin-2-yl)-7-methyl-4- oxo-4H-pyrido[1,2-a]pyrimidin-9- yl)azetidine-3-carboxylic acid |
| 11-426 | 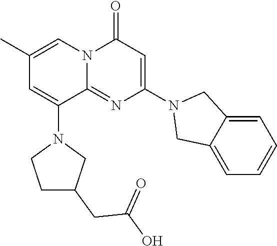 [SMILES: Cc1cc(N2CCC(CC(=O)O)C2)c2nc(N3Cc4ccccc4C3)cc(=O)n2c1] | 2-(1-(2-(isoindolin-2-yl)-7-methyl- 4-oxo-4H-pyrido[1,2-a]pyrimidin- 9-yl)pyrrolidin-3-yl)acetic acid |
| 11-427 | 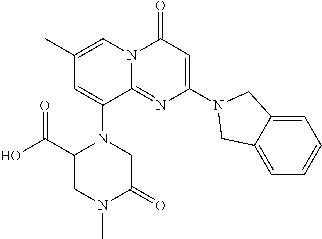 [SMILES: Cc1cc(N2CC(=O)N(C)CC2C(=O)O)c2nc(N3Cc4ccccc4C3)cc(=O)n2c1] | 1-(2-(isoindolin-2-yl)-7-methyl-4- oxo-4H-pyrido[1,2-a]pyrimidin-9- yl)-4-methyl-5-oxopiperazine-2- carboxylic acid |
| 11-428 | 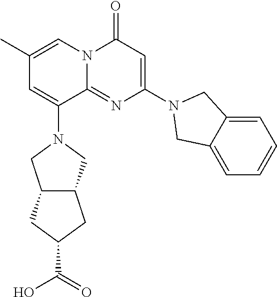 [SMILES: Cc1cc(N2C[C@H]3C[C@@H](C(=O)O)C[C@H]3C2)c2nc(N3Cc4ccccc4C3)cc(=O)n2c1] | (3aR,5r,6aS)-2-(2-(isoindolin-2- yl)-7-methyl-4-oxo-4H- pyrido[1,2-a]pyrimidin-9- yl)octahydrocyclopenta[c]pyrrole- 5-carboxylic acid |
| 11-429 | 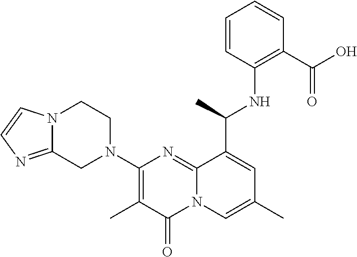 [SMILES: Cc1cc([C@@H](C)Nc2ccccc2C(=O)O)c2nc(N3CCn4ccnc4C3)c(C)c(=O)n2c1] | (R)-2-((1-(2-(5,6- dihydroimidazo[1,2-a]pyrazin- 7(8H)-yl)-3,7-dimethyl-4-oxo- 4H-pyrido[1,2-a]pyrimidin-9- yl)ethyl)amino)benzoic acid |
| 11-430 | 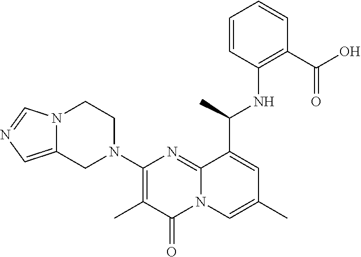 [SMILES: Cc1cc([C@@H](C)Nc2ccccc2C(=O)O)c2nc(N3CCn4cncc4C3)c(C)c(=O)n2c1] | (R)-2-((1-(2-(5,6- dihydroimidazo[1,5-a]pyrazin- 7(8H)-yl)-3,7-dimethyl-4-oxo- 4H-pyrido[1,2-a]pyrimidin-9- yl)ethyl)amino)benzoic acid |
| 11-431 | 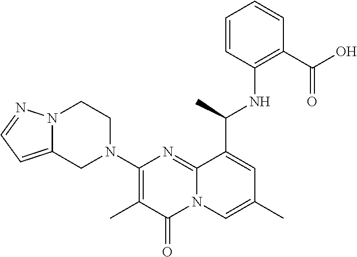 [SMILES: Cc1cc([C@@H](C)Nc2ccccc2C(=O)O)c2nc(N3CCn4nccc4C3)c(C)c(=O)n2c1] | (R)-2-((1-(2-(6,7- dihydropyrazolo[1,5-a]pyrazin- 5(4H)-yl)-3,7-dimethyl-4-oxo- 4H-pyrido[1,2-a]pyrimidin-9- yl)ethyl)amino)benzoic acid |
| 11-432 | 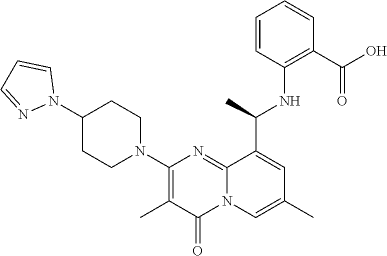 [SMILES: Cc1cc([C@@H](C)Nc2ccccc2C(=O)O)c2nc(N3CCC(n4cccn4)CC3)c(C)c(=O)n2c1] | (R)-2-((1-(2-(4-(1H-pyrazol-1- yl)piperidin-1-yl)-3,7-dimethyl-4- oxo-4H-pyrido[1,2-a]pyrimidin-9- yl)ethyl)amino)benzoic acid |
| 11-433 | 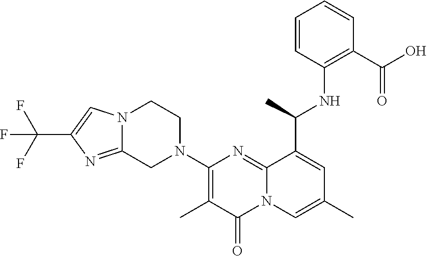 [SMILES: Cc1cc([C@@H](C)Nc2ccccc2C(=O)O)c2nc(N3CCn4cc(C(F)(F)F)nc4C3)c(C)c(=O)n2c1] | (R)-2-((1-(3,7-dimethyl-4-oxo-2- (2-(trifluoromethyl)-5,6- dihydroimidazo[1,2-a]pyrazin- 7(8H)-yl)-4H- pyrido[1,2-a]pyrimidin-9- yl)ethyl)amino)benzoic acid |
| 11-434 | 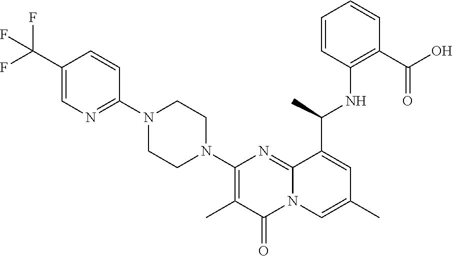 [SMILES: Cc1cc([C@@H](C)Nc2ccccc2C(=O)O)c2nc(N3CCN(c4ccc(C(F)(F)F)cn4)CC3)c(C)c(=O)n2c1] | (R)-2-((1-(3,7-dimethyl-4-oxo-2- (4-(5-(trifluoromethyl)pyridin-2- yl)piperazin-1-yl)- 4H-pyrido[1,2-a]pyrimidin-9- yl)ethyl)amino)benzoic acid |
| 11-435 | 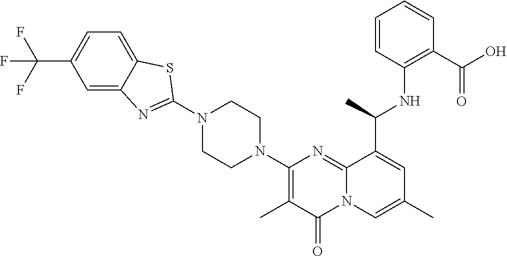 [SMILES: Cc1cc([C@@H](C)Nc2ccccc2C(=O)O)c2nc(N3CCN(c4nc5cc(C(F)(F)F)ccc5s4)CC3)c(C)c(=O)n2c1] | (R)-2-((1-(3,7-dimethyl-4-oxo-2- (4-(5- (trifluoromethyl)benzo[d]thiazol- 2-yl)piperazin-1-yl)- 4H-pyrido[1,2-a]pyrimidin-9- yl)ethyl)amino)benzoic acid |
| 11-436 | 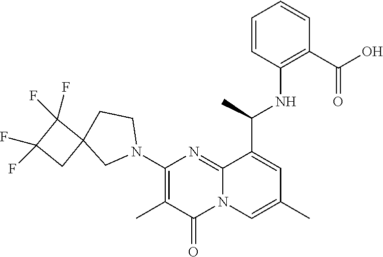 [SMILES: Cc1cc([C@@H](C)Nc2ccccc2C(=O)O)c2nc(N3CCC4(C3)CC(F)(F)C4(F)F)c(C)c(=O)n2c1] | 2-(((1R)-1-(3,7-dimethyl-4-oxo-2- (1,1,2,2-tetrafluoro-6- azaspiro[3.4]octan-6-yl)- 4H-pyrido[1,2-a]pyrimidin-9- yl)ethyl)amino)benzoic acid |
| 11-437 | 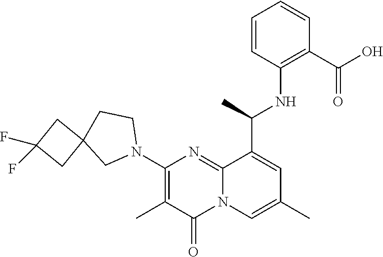 [SMILES: Cc1cc([C@@H](C)Nc2ccccc2C(=O)O)c2nc(N3CCC4(C3)CC(F)(F)C4)c(C)c(=O)n2c1] | (R)-2-((1-(2-(2,2-difluoro-6- azaspiro[3.4]octan-6-yl)-3,7- dimethyl-4-oxo- 4H-pyrido[1,2-a]pyrimidin-9- yl)ethyl)amino)benzoic acid |
| 11-438 | 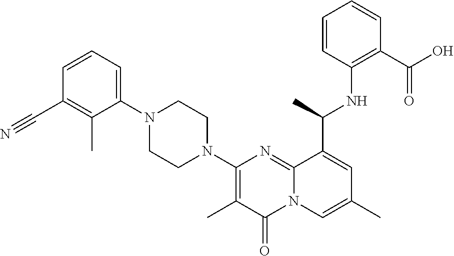 [SMILES: Cc1cc([C@@H](C)Nc2ccccc2C(=O)O)c2nc(N3CCN(c4cccc(C#N)c4C)CC3)c(C)c(=O)n2c1] | (R)-2-((1-(2-(4-(3-cyano-2-methylphenyl) piperazin-1-yl)-3,7-dimethyl-4-oxo- 4H-pyrido[1,2-a]pyrimidin-9- yl)ethyl)amino)benzoic acid |
| 11-439 | 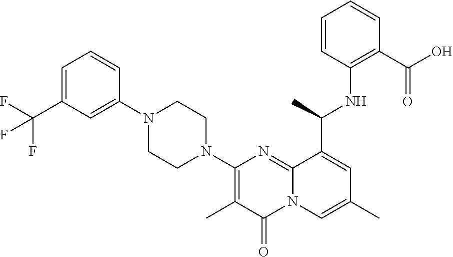 [SMILES: Cc1cc([C@@H](C)Nc2ccccc2C(=O)O)c2nc(N3CCN(c4cccc(C(F)(F)F)c4)CC3)c(C)c(=O)n2c1] | (R)-2-((1-(3,7-dimethyl-4-oxo-2-(4-(3- (trifluoromethyl)phenyl)piperazin- 1-yl)-4H-pyrido[1,2-a]pyrimidin-9- yl)ethyl)amino)benzoic acid |
| 11-440 | 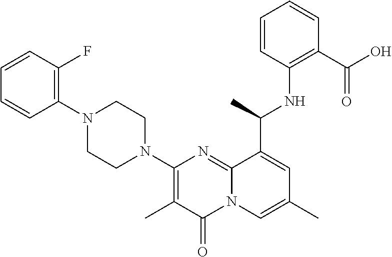 [SMILES: Cc1cc([C@@H](C)Nc2ccccc2C(=O)O)c2nc(N3CCN(c4ccccc4F)CC3)c(C)c(=O)n2c1] | (R)-2-((1-(2-(4-(2- fluorophenyl)piperazin-1-yl)-3,7- dimethyl-4-oxo- 4H-pyrido[1,2-a]pyrimidin-9- yl)ethyl)amino)benzoic acid |
| 11-441 | 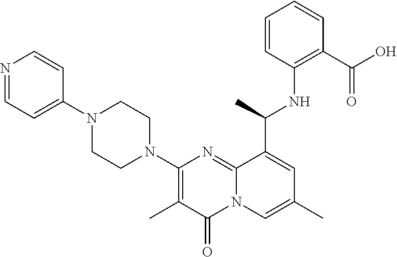 [SMILES: Cc1cc([C@@H](C)Nc2ccccc2C(=O)O)c2nc(N3CCN(c4ccncc4)CC3)c(C)c(=O)n2c1] | (R)-2-((1-(3,7-dimethyl-4-oxo-2- (4-(pyridin-4-yl)piperazin-1-yl)- 4H-pyrido[1,2-a]pyrimidin-9- yl)ethyl)amino)benzoic acid |
| 11-442 | 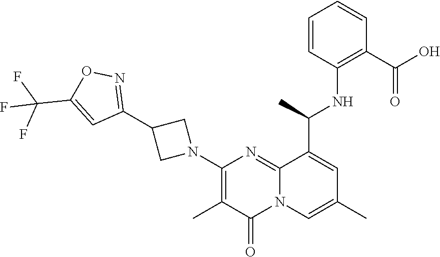 [SMILES: Cc1cc([C@@H](C)Nc2ccccc2C(=O)O)c2nc(N3CC(c4cc(C(F)(F)F)on4)C3)c(C)c(=O)n2c1] | (R)-2-((1-(3,7-dimethyl-4-oxo-2- (3-(5-(trifluoromethyl)isoxazol-3- yl)azetidin-1-yl)- 4H-pyrido[1,2-a]pyrimidin-9- yl)ethyl)amino)benzoic acid |
| 11-443 | 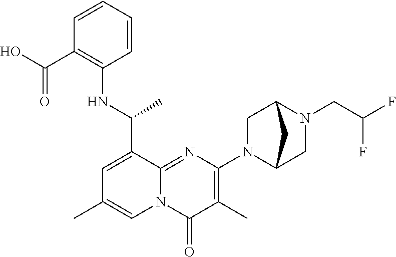 [SMILES: Cc1cc([C@@H](C)Nc2ccccc2C(=O)O)c2nc(N3C[C@H]4C[C@@H]3CN4CC(F)F)c(C)c(=O)n2c1] | 2-(((R)-1-(2-((1R,4R)-5-(2,2- difluoroethyl)-2,5- diazabicyclo[2.2.1]heptan-2-yl)- 3,7-dimethyl-4-oxo- 4H-pyrido[1,2-a]pyrimidin-9- yl)ethyl)amino)benzoic acid |
| 11-444 | 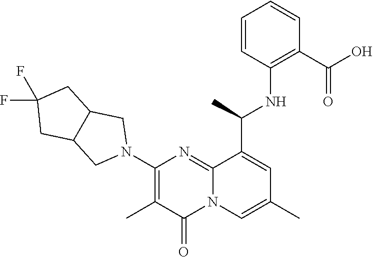 [SMILES: Cc1cc([C@@H](C)Nc2ccccc2C(=O)O)c2nc(N3CC4CC(F)(F)CC4C3)c(C)c(=O)n2c1] | 2-(((1R)-1-(2-(5,5- difluorohexahydrocyclopenta[c] pyrrol-2(1H)-yl)-3,7-dimethyl-4- oxo-4H-pyrido[1,2-a]pyrimidin-9- yl)ethyl)amino)benzoic acid |
| 11-445 | 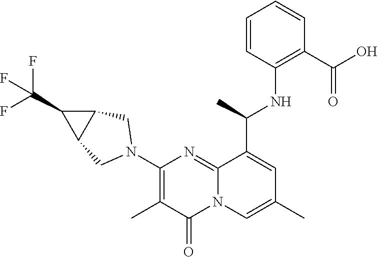 [SMILES: Cc1cc([C@@H](C)Nc2ccccc2C(=O)O)c2nc(N3C[C@@H]4[C@H](C3)[C@H]4C(F)(F)F)c(C)c(=O)n2c1] | 2-(((R)-1-(3,7-dimethyl-4-oxo-2- ((1R,5S,6R)-6-(trifluoromethyl)- 3-azabicyclo[3.1.0]hexan-3-yl)- 4H-pyrido[1,2-a]pyrimidin-9- yl)ethyl)amino)benzoic acid |
| 11-446 | 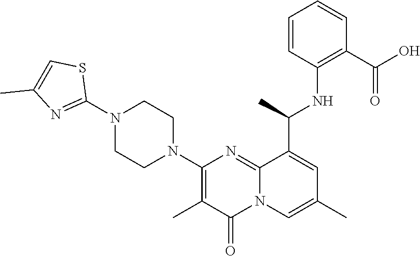 [SMILES: Cc1cc([C@@H](C)Nc2ccccc2C(=O)O)c2nc(N3CCN(c4nc(C)cs4)CC3)c(C)c(=O)n2c1] | (R)-2-((1-(3,7-dimethyl-2-(4-(4- methylthiazol-2-yl)piperazin-1- yl)-4-oxo- 4H-pyrido[1,2-a]pyrimidin-9- yl)ethyl)amino)benzoic acid |
| 11-447 | 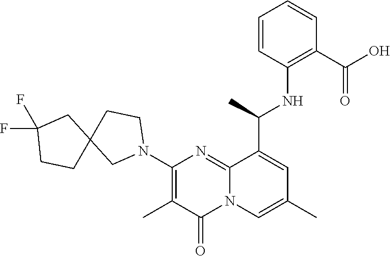 [SMILES: Cc1cc([C@@H](C)Nc2ccccc2C(=O)O)c2nc(N3CCC4(CCC(F)(F)C4)C3)c(C)c(=O)n2c1] | 2-(((1R)-1-(2-(7,7-difluoro-2- azaspiro[4.4]nonan-2-yl)-3,7- dimethyl-4-oxo- 4H-pyrido[1,2-a]pyrimidin-9- yl)ethyl)amino)benzoic acid |

### Example A

This Example illustrates that exemplary compounds of the present invention inhibit the formation of phospho-AKT (pAKT) in a cell.

The ability of a compound of Formula (I) to inhibit the formation of pAKT was measured using alphaLISA Surefire Ultra AKT 1/2/3 (pS473) Assay Kit (#ALSU-PAKT-B50K) was obtained from Perkin Elmer (Waltham, MA).

To prepare assay plates for pAKT alphaLISA assays, cells were trypsinized, resuspended in fresh media, and viable cells were counted utilizing trypan blue exclusion. Prior to seeding, cells were washed with PBS and resuspended in HBSS (Gibco, #14025092). T47D (12,000/w), SKBR3(12,000/w), or MKN1(24,000/w) cells were seeded at 12 μl per well in a solid white flat bottom 384 cell culture plate (Perkin Elmer #6007680).

Immediately after seeding, cells were dosed using an Echo Liquid Handler (Beckman Coulter) with compounds at a 10.4 μm starting concentration and serially diluted (1:4) for a total of 10 concentrations. 14 vehicle (DMSO) and 14 positive control (Alpelisib @ 3.125 μm) wells were included on each assay plate. Cells were incubated with the compounds (solubilized in DMSO) for approximately one hour or 24 hours at 37° C. After one hour (or 24 hours) of treatment, cells were lysed with 3 μl of the 5× lysis buffer (provided and incubated at room temperature for 15 min on a microtiter plate shaker). Once cells were sufficiently lysed, 7.5 μl of the acceptor bead mix (made using the manufacturers recommended dilutions) was added to each well and left on the microtiter plate shaker for 1 min prior to incubating plates at room temperature for one hour, in the absence of light. After one hour of incubation with the acceptor bead mix, 7.5 μl of the donor bead mix (made using the manufacturers recommended dilutions) was added to each well and left on the microtiter plate shaker for 1 min prior to incubating plates at room temperature overnight in the absence of light. Plates were then imaged the following day using a CLARIOstar microplate reader (BMG Labtech, Germany).

Percent of control values were calculated by subtracting the average signal from the positive control (alpelisib) treated wells from all treated wells (including DMSO control wells) and then divided by the average signal from the vehicle DMSO treated control wells. Percent of vehicle control values were plotted as log (inhibitor) vs. response-Variable slope (four parameters) for curve fitting and IC50 values were determined using XLfit.

The results are shown in Tables below. Key: N.D. =not determined

TABLE A

| | T47D pAKT |
|---|---|
| | (H1047R mutant) |
| Example # | IC50 (nM) |
| 1 | >10000 |
| 2 | 14.01 |
| 3-1 | 14.89 |
| 3-2 | 1906 |
| 3-3 | 2735 |
| 3-4 | 2279 |
| 3-5 | 1805 |
| 3-6 | >10000 |
| 3-7 | >10000 |
| 3-8 | 2997 |
| 3-9 | >10000 |
| 3-10 | 2513 |
| 3-11 | >10000 |
| 3-12 | 1967 |
| 3-13 | 1064 |
| 3-14 | 4535 |
| 3-15 | >10000 |
| 3-16 | >10000 |
| 3-17 | >10000 |
| 3-18 | 1391 |
| 3-19 | 38.33 |
| 3-20 | 1122 |
| 3-21 | >10000 |
| 3-22 | 70.47 |
| 4-1 | >10000 |
| 4-2 | >10000 |
| 4-3 | >10000 |
| 4-4 | >10000 |
| 5-1 | 30.02 |
| 5-2 | >10000 |
| 5-3 | 32.12 |
| 5-4 | 13.48 |
| 5-5 | 1206 |
| 5-6 | 28.14 |
| 5-7 | 34.1 |
| 5-8 | 17 |
| 5-9 | 21 |
| 5-10 | 40.39 |
| 5-11 | 10.9 |
| 5-9-1 | 11.72 |
| 5-9-2 | 3010 |
| 6-1 | 3371 |
| 6-2 | 93.21 |
| 7-1 | 13.28 |
| 7-2 | >100000 |
| 8-1 | 54 |
| 9-1 | 21.21 |
| 9-2 | 33.9 |
| 9-3 | 128.8 |
| 9-4 | 25.88 |
| 10-1 | 2.266 |
| 10-2 | |
| 10-3 | |
| 10-4 | |
| 10-5 | 0.4252 |
| 10-6 | |
| 10-7 | |
| 10-8 | |
| 10-9 | |
| 10-10 | 1.214 |
| 10-11 | 1.712 |
| 10-12 | 0.6394 |
| 10-13 | 1.001 |
| 10-14 | 2.339 |
| 10-15 | 0.8534 |
| 10-16 | |
| 10-17 | |
| 10-18 | 2.006 |
| 10-19 | 0.2445 |
| 10-20 | 1.596 |
| 10-21 | 2.339 |
| 10-22 | 3.846 |
| 10-23 | 1.354 |
| 10-24 | 5.486 |
| 10-25 | 4.691 |
| 10-26 | |
| 10-27 | 2.102 |
| 10-28 | |
| 10-29 | 0.5399 |
| 10-30 | 3.617 |
| 10-31 | 6.395 |
| 10-32 | 5.779 |
| 10-33 | 2.194 |
| 10-34 | 1.382 |
| 10-35 | 4.717 |
| 10-36 | 3.804 |
| 10-37 | 2.192 |
| 10-38 | 1.967 |
| 10-39 | 1.704 |
| 10-40 | 1.91 |
| 10-41 | 7.177 |
| 10-42 | 3.088 |
| 10-43 | 2.518 |
| 10-44 | 1.471 |
| 10-45 | 2.943 |
| 10-46 | |
| 10-47 | |
| 10-48 | 2.958 |
| 10-49 | 2.111 |
| 10-50 | 1.336 |
| 10-51 | 1.925 |
| 10-52 | 2.497 |
| 10-53 | 4.351 |
| 10-54 | 3.361 |
| 10-55 | 3.345 |
| 10-56 | 3.952 |
| 10-57 | 5.173 |
| 10-58 | 4.019 |
| 10-59 | 1.376 |
| 10-60 | 2.618 |
| 10-61 | 7.704 |
| 10-62 | 4.845 |
| 10-63 | 144.2 |
| 10-64 | 16.14 |
| 10-65 | 3.25 |
| 10-66 | 1367 |
| 10-67 | 14.06 |
| 10-68 | 374.8 |
| 10-69 | 3.695 |
| 10-70 | >10000 |
| 10-71 | >10000 |
| 10-72 | 1.33 |
| 10-73 | 3.29 |
| 10-74 | 3.227 |
| 10-75 | 1.051 |
| 10-76 | 1.703 |
| 10-77 | 3.804 |
| 10-78 | 2.205 |
| 10-79 | 0.2989 |
| 10-80 | 1.982 |
| 10-81 | 2.527 |
| 10-82 | 2.597 |
| 10-83 | |
| 10-84 | 2.979 |
| 10-85 | 1.998 |
| 10-86 | 15.51 |
| 10-87 | 2.234 |
| 10-88 | 3.774 |
| 10-89 | 8.137 |
| 10-90 | 0.5964 |
| 10-91 | 2.554 |
| 10-92 | 1.712 |
| 10-93 | 4.303 |
| 10-94 | 2.019 |
| 10-95 | 1.383 |
| 10-96 | 0.9053 |
| 10-97 | 2.037 |
| 10-98 | 2.116 |
| 10-99 | 0.9776 |
| 10-100 | 3.819 |
| 10-101 | 5.782 |
| 10-102 | 3.705 |
| 10-103 | 2.052 |
| 10-104 | |
| 10-105 | |
| 10-106 | |
| 10-107 | 6.722 |
| 10-108 | 2.893 |
| 10-109 | 2.672 |
| 10-110 | 1.05 |
| 10-111 | 4.823 |
| 10-112 | 6.232 |
| 10-113 | 3.024 |
| 10-114 | 2.579 |
| 10-115 | 58.43 |
| 10-116 | 1.58 |
| 10-117 | 2.157 |
| 10-118 | 2.341 |
| 10-119 | |
| 10-120 | 2.254 |
| 10-121 | |
| 10-122 | 2.036 |
| 10-123 | 2.386 |
| 10-124 | 1.593 |
| 10-125 | 3.133 |
| 10-126 | 2.644 |
| 10-127 | 1.191 |
| 10-128 | |
| 10-129 | |
| 10-130 | |
| 10-131 | 1.856 |
| 10-132 | |
| 10-133 | |
| 10-134 | |
| 10-135 | |
| 10-136 | |
| 10-137 | |
| 10-138 | |
| 10-139 | |
| 10-140 | 571.9 |
| 10-141 | 4.1 |
| 10-142 | |
| 10-143 | |
| 10-144 | |
| 10-145 | |
| 10-146 | |
| 10-147 | |
| 10-148 | |

Inhibition of pAKT in T47D (PI3Kα H1047R mutant) cells by Exemplary Compounds of Formula (I)

TABLE B
Inhibition of pAKT in MKN1 (PI3Kα E545K
mutant) cells by Exemplary Compounds of Formula (I)

| | MKN1 (E545K) |
|---|---|
| Example # | pAKT IC₅₀ (nM) |
| 1 | >10000 |
| 2 | |
| 3-1 | 2499 |
| 3-2 | 754.7 |
| 3-3 | >10000 |
| 3-4 | 8830 |
| 3-5 | >10000 |
| 3-6 | >10000 |
| 3-7 | >10000 |
| 3-8 | >10000 |
| 3-9 | >10000 |
| 3-10 | >10000 |
| 3-11 | >10000 |
| 3-12 | >10000 |
| 3-13 | 3988 |
| 3-14 | 2913 |
| 3-15 | >10000 |
| 3-16 | >10000 |
| 3-17 | >10000 |
| 3-18 | >10000 |
| 3-19 | 1190 |
| 3-20 | >10000 |
| 3-21 | >10000 |
| 3-22 | 3252.00 |
| 4-1 | >10000 |
| 4-2 | >10000 |
| 4-3 | >10000 |
| 4-4 | >10000 |
| 5-1 | 1388 |
| 5-2 | >10000 |
| 5-3 | >10000 |
| 5-4 | 1950 |
| 5-5 | >10000 |
| 5-6 | 6520 |
| 5-7 | >10000 |
| 5-8 | >10000 |
| 5-9 | 3502.00 |
| 5-9-1 | 270.9 |
| 5-9-2 | >10000 |
| 5-10 | >10000 |
| 5-11 | 3059.00 |
| 6-1 | >10000 |
| 6-2 | >10000 |
| 7-1 | 6423 |
| 7-2 | |
| 8-1 | 5535 |
| 9-1 | >10000 |
| 9-2 | >10000 |
| 9-3 | >10000 |
| 9-4 | >10000 |

TABLE C
Inhibition of pAKT in SKBR3 (PI3Kα wild
type) cells by Exemplary Compounds of Formula (I)

| | SKBR3 (WT) |
|---|---|
| | pAKT IC50 (nM) |
| | (H1047R mutant) |
| Example # | IC50 (nM) |
| 1 | >10000 |
| 2 | >10000 |
| 3-1 | >10000 |
| 3-2 | >10000 |
| 3-3 | >10000 |
| 3-4 | >10000 |
| 3-5 | >10000 |
| 3-6 | 2666 |
| 3-7 | 8028 |
| 3-8 | >10000 |
| 3-9 | 582.8 |
| 3-10 | >10000 |
| 3-11 | >10000 |
| 3-12 | 44.3 |
| 3-13 | >10000 |
| 3-14 | >10000 |
| 3-15 | >10000 |
| 3-16 | >10000 |
| 3-17 | >10000 |
| 3-18 | >10000 |
| 3-19 | 2804 |
| 3-20 | >10000 |
| 3-21 | |
| 3-22 | >10000 |
| 4-1 | >10000 |
| 4-2 | >10000 |
| 4-3 | >10000 |
| 4-4 | >10000 |
| 5-1 | 1815 |
| 5-2 | >10000 |
| 5-3 | 3539 |
| 5-4 | 7069 |
| 5-5 | >10000 |
| 5-6 | 9053 |
| 5-7 | >10000 |
| 5-8 | >10000 |
| 5-9 | 683.00 |
| 5-10 | 6119 |
| 5-11 | >10000 |
| 5-9-1 | 4551 |
| 5-9-2 | >10000 |
| 6-1 | >10000 |
| 6-2 | >10000 |
| 7-1 | 6651 |
| 7-2 | >10000 |
| 8-1 | 932 |
| 9-1 | >10000 |
| 9-2 | >10000 |
| 9-3 | >10000 |
| 9-4 | >10000 |
| 10-1 | |
| 10-2 | |
| 10-3 | |
| 10-4 | |
| 10-5 | |
| 10-6 | |
| 10-7 | |
| 10-8 | |
| 10-9 | |
| 10-10 | |
| 10-11 | |
| 10-12 | |
| 10-13 | |
| 10-14 | |
| 10-15 | |
| 10-16 | |
| 10-17 | |
| 10-18 | |
| 10-19 | |
| 10-20 | |
| 10-21 | |
| 10-22 | |
| 10-23 | 1201 |
| 10-24 | |
| 10-25 | |
| 10-26 | |
| 10-27 | 779.5 |
| 10-28 | |
| 10-29 | |
| 10-30 | |
| 10-31 | |
| 10-32 | |
| 10-33 | |
| 10-34 | |
| 10-35 | |
| 10-36 | |
| 10-37 | |
| 10-38 | |
| 10-39 | |
| 10-40 | |
| 10-41 | |
| 10-42 | |
| 10-43 | |
| 10-44 | |
| 10-45 | |
| 10-46 | |
| 10-47 | |
| 10-48 | |
| 10-49 | |
| 10-50 | |
| 10-51 | |
| 10-52 | |
| 10-53 | |
| 10-54 | |
| 10-55 | |
| 10-56 | |
| 10-57 | |
| 10-58 | |
| 10-59 | |
| 10-60 | |
| 10-61 | 3652 |
| 10-62 | 8652 |
| 10-63 | >10000 |
| 10-64 | 846.9 |
| 10-65 | 681 |
| 10-66 | >10000 |
| 10-67 | >10000 |
| 10-68 | >10000 |
| 10-69 | 1803 |
| 10-70 | >10000 |
| 10-71 | >10000 |
| 10-72 | 2480 |
| 10-73 | 1034 |
| 10-74 | 1465 |
| 10-75 | |
| 10-76 | |
| 10-77 | |
| 10-78 | |
| 10-79 | |
| 10-80 | |
| 10-81 | |
| 10-82 | |
| 10-83 | |
| 10-84 | |
| 10-85 | |
| 10-86 | |
| 10-87 | |
| 10-88 | |
| 10-89 | |
| 10-90 | |
| 10-91 | |
| 10-92 | |
| 10-93 | |
| 10-94 | |
| 10-95 | |
| 10-96 | |
| 10-97 | |
| 10-98 | |
| 10-99 | 2261 |
| 10-100 | |
| 10-101 | |
| 10-102 | |
| 10-103 | |
| 10-104 | |
| 10-105 | |
| 10-106 | |
| 10-107 | |
| 10-108 | |
| 10-109 | |
| 10-110 | |
| 10-111 | |
| 10-112 | |
| 10-113 | |
| 10-114 | |
| 10-115 | |
| 10-116 | |
| 10-117 | |
| 10-118 | |
| 10-119 | |
| 10-120 | |
| 10-121 | |
| 10-122 | |
| 10-123 | |
| 10-124 | |
| 10-125 | |
| 10-126 | |
| 10-127 | |
| 10-128 | |
| 10-129 | |
| 10-130 | |
| 10-131 | |
| 10-132 | |
| 10-133 | |
| 10-134 | |
| 10-135 | |
| 10-136 | |
| 10-137 | |
| 10-138 | |
| 10-139 | |
| 10-140 | |
| 10-141 | |
| 10-142 | |
| 10-143 | |
| 10-144 | |
| 10-145 | |
| 10-146 | |
| 10-147 | |
| 10-148 | |

### Example B

This Example illustrates that exemplary compounds of the present invention decrease the viability of cells.

The ability of a compound of Formula (I) to decrease the viability of cells was measured using CellTiter-Glo 2.0 (CTG) Luminescent Cell Viability Assay (#G9241) obtained from Promega (Madison, WI).

To prepare assay plates for viability assays, cells were trypsinized, resuspended in fresh media, and viable cells were counted utilizing trypan blue exclusion. T47D, SKBR3, or MKN1 cells were seeded at 1000 cells in 30 μl per well in a solid white flat bottom 384 cell culture plate (Perkin Elmer #6007680) and incubated at 37° C. overnight.

Assay day 1, cells were dosed using an Echo Liquid Handler (Beckman Coulter) with compounds at a 10 um starting concentration and serially diluted (1:4) for a total of 10 concentrations. Cells were incubated for approximately 72 hours with the compounds (solubilized in DMSO) at 37° C. After 72 hours of treatment, cell plates were equilibrated to room temperature before adding 15 μl of CTG to each well, plates were then covered in aluminum foil to protect from light, incubated at room temperature for 30 minutes on a microtiter plate shaker, and luminescence readings were collected using a CLARIOstar microplate reader (BMG Labtech, Germany). Percent of vehicle control values were plotted as log (inhibitor) vs. response-Variable slope (four parameters) for curve fitting and IC₅₀ values were determined using XLfit.

TABLE D
Viability in T47D (PI3Kα H1047R mutant)
cells by Exemplary Compounds of Formula (I)

| | T47D (H1047R mutant) |
|---|---|
| | Viability IC50 |
| Example # | (nM) |
| 1 | >10000 |
| 2 | 2973 |
| 3-1 | 1267 |
| 3-2 | >10000 |
| 3-3 | >10000 |
| 3-4 | |
| 3-5 | >10000 |
| 3-6 | >10000 |
| 3-7 | >10000 |
| 3-8 | >10000 |
| 3-9 | >10000 |
| 3-10 | >10000 |
| 3-11 | >10000 |
| 3-12 | 5762 |
| 3-13 | >10000 |
| 3-14 | 1358 |
| 3-15 | >10000 |
| 3-16 | >10000 |
| 3-17 | >10000 |
| 3-18 | 4204 |
| 3-19 | 367.5 |
| 3-20 | >10000 |
| 3-21 | |
| 3-22 | 1640 |
| 4-1 | >10000 |
| 4-2 | >10000 |
| 4-3 | >10000 |
| 4-4 | >10000 |
| 5-1 | 461.1 |
| 5-2 | 9031 |
| 5-3 | >10000 |
| 5-4 | 980.1 |
| 5-5 | 8020 |
| 5-6 | 3801 |
| 5-7 | 2627 |
| 5-8 | 3785 |
| 5-9 | 430 |
| 5-10 | 1799 |
| 5-11 | 731.4 |
| 5-9-1 | 316.2 |
| 5-9-2 | 9681 |
| 6-1 | >10000 |
| 6-2 | >10000 |
| 7-1 | 3143 |
| 7-2 | >10000 |
| 8-1 | 166.2 |
| 9-1 | 2585 |
| 9-2 | >10000 |
| 9-3 | 4448 |
| 9-4 | 9972 |
| 10-1 | 38.54 |
| 10-2 | 4.723 |
| 10-3 | 8.116 |
| 10-4 | 12.34 |
| 10-5 | 13.56 |
| 10-6 | 14.97 |
| 10-7 | 16.94 |
| 10-8 | 24.24 |
| 10-9 | 24.76 |
| 10-10 | 29.29 |
| 10-11 | 30.5 |
| 10-12 | 31.02 |
| 10-13 | 32.84 |
| 10-14 | 33.08 |
| 10-15 | 36.36 |
| 10-16 | 37.33 |
| 10-17 | 38.41 |
| 10-18 | 38.43 |
| 10-19 | 41.39 |
| 10-20 | 42.71 |
| 10-21 | 45.79 |
| 10-22 | 47.96 |
| 10-23 | 48.34 |
| 10-24 | 48.58 |
| 10-25 | 49.61 |
| 10-26 | 50.45 |
| 10-27 | 52.04 |
| 10-28 | 52.99 |
| 10-29 | 53.91 |
| 10-30 | 55 |
| 10-31 | 55.11 |
| 10-32 | 67.35 |
| 10-33 | 56.55 |
| 10-34 | 56.56 |
| 10-35 | 56.66 |
| 10-36 | 56.96 |
| 10-37 | 57.39 |
| 10-38 | 57.86 |
| 10-39 | 59.02 |
| 10-40 | 60.02 |
| 10-41 | 60.16 |
| 10-42 | 61.51 |
| 10-43 | 62.24 |
| 10-44 | 62.47 |
| 10-45 | 64.21 |
| 10-46 | 65.01 |
| 10-47 | 65.04 |
| 10-48 | 66.33 |
| 10-49 | 66.36 |
| 10-50 | 66.47 |
| 10-51 | 68.04 |
| 10-52 | 68.06 |
| 10-53 | 77.25 |
| 10-54 | 72.22 |
| 10-55 | 72.39 |
| 10-56 | 73.16 |
| 10-57 | 73.31 |
| 10-58 | 73.3 |
| 10-59 | 111.7 |
| 10-60 | 98.79 |
| 10-61 | 183 |
| 10-62 | 566.3 |
| 10-63 | 3725 |
| 10-64 | 627.5 |
| 10-65 | 246.3 |
| 10-66 | >10000 |
| 10-67 | 304.1 |
| 10-68 | >10000 |
| 10-69 | 164.6 |
| 10-70 | 4680 |
| 10-71 | 4893 |
| 10-72 | 150.8 |
| 10-73 | 135.2 |
| 10-74 | 120.5 |
| 10-75 | 110.1 |
| 10-76 | 108.8 |
| 10-77 | 107.3 |
| 10-78 | 74.04 |
| 10-79 | 77.51 |
| 10-80 | 75.02 |
| 10-81 | 78.47 |
| 10-82 | 78.51 |
| 10-83 | 78.67 |
| 10-84 | 79 |
| 10-85 | 79.45 |
| 10-86 | 80.05 |
| 10-87 | 80.22 |
| 10-88 | 80.27 |
| 10-89 | 81.65 |
| 10-90 | 82.38 |
| 10-91 | 82.54 |
| 10-92 | 82.84 |
| 10-93 | 83.22 |
| 10-94 | 83.63 |
| 10-95 | 110.7 |
| 10-96 | 84.4 |
| 10-97 | 85.21 |
| 10-98 | 85.76 |
| 10-99 | 86.23 |
| 10-100 | 86.23 |
| 10-101 | 86.78 |
| 10-102 | 87 |
| 10-103 | 87.2 |
| 10-104 | 87.75 |
| 10-105 | 89.36 |
| 10-106 | 91.11 |
| 10-107 | 92.5 |
| 10-108 | 93.63 |
| 10-109 | 93.72 |
| 10-110 | 94.1 |
| 10-111 | 96.37 |
| 10-112 | 96.69 |
| 10-113 | 98.01 |
| 10-114 | 98.47 |
| 10-115 | 98.81 |
| 10-116 | 99.98 |
| 10-117 | 101 |
| 10-118 | 101.3 |
| 10-119 | 102.4 |
| 10-120 | 102.8 |
| 10-121 | 102.9 |
| 10-122 | 103.3 |
| 10-123 | 104.3 |
| 10-124 | 105.3 |
| 10-125 | 106.7 |
| 10-126 | 109.3 |
| 10-127 | 74.38 |
| 10-128 | 43.18 |
| 10-129 | 46.68 |
| 10-130 | 46.93 |
| 10-131 | 48.83 |
| 10-132 | 51.03 |
| 10-133 | 55.44 |
| 10-134 | 56.57 |
| 10-135 | 61.05 |
| 10-136 | 64.23 |
| 10-137 | 69.51 |
| 10-138 | 71.55 |
| 10-139 | 74.34 |
| 10-140 | 75.82 |
| 10-141 | 162.6 |
| 10-142 | 80.1 |
| 10-143 | 83.1 |
| 10-144 | 91.34 |
| 10-145 | 98.45 |
| 10-146 | 100.3 |
| 10-147 | 107 |
| 10-148 | 108.5 |
| 11-1 | |
| 11-2 | |
| 11-3 | |
| 11-4 | |
| 11-5 | |
| 11-6 | |
| 11-7 | |
| 11-8 | |
| 11-9 | |
| 11-10 | |
| 11-11 | |
| 11-12 | |
| 11-13 | 323.7 |
| 11-14 | 232.9 |
| 11-15 | 156.2 |
| 11-16 | 389.1 |
| 11-17 | 795.6 |
| 11-18 | 415.7 |
| 11-19 | 173 |
| 11-20 | 316.3 |
| 11-21 | 238.6 |
| 11-22 | 191.8 |
| 11-23 | 139.4 |
| 11-24 | 123 |
| 11-25 | |
| 11-26 | 215.9 |
| 11-27 | 1597 |
| 11-28 | 186.6 |
| 11-29 | 244.1 |
| 11-30 | 401.3 |
| 11-31 | 328.1 |
| 11-32 | 303.3 |
| 11-33 | 309.6 |
| 11-34 | 214.8 |
| 11-35 | 136.3 |
| 11-36 | 390.2 |
| 11-37 | 816.4 |
| 11-38 | 194.4 |
| 11-39 | 113.6 |
| 11-40 | |
| 11-41 | |
| 11-42 | 172.1 |
| 11-43 | 371.7 |
| 11-44 | 9550 |
| 11-45 | 2974 |
| 11-46 | 149.8 |
| 11-47 | 242 |
| 11-48 | 853.9 |
| 11-49 | 203.6 |
| 11-50 | 217.2 |
| 11-51 | >10000 |
| 11-52 | 6642 |
| 11-53 | 2525 |
| 11-54 | 324.8 |
| 11-55 | 219.1 |
| 11-56 | 263.9 |
| 11-57 | 132.1 |
| 11-58 | 170 |
| 11-59 | 273.4 |
| 11-60 | 157.8 |
| 11-61 | 145.1 |
| 11-62 | 172.7 |
| 11-63 | 136.2 |
| 11-64 | 130.6 |
| 11-65 | 145.7 |
| 11-66 | 231 |
| 11-67 | 144.4 |
| 11-68 | 507.6 |
| 11-69 | 167.8 |
| 11-70 | 267.5 |
| 11-71 | 387.2 |
| 11-72 | 440.7 |
| 11-73 | 272.3 |
| 11-74 | 1980 |
| 11-75 | 1569 |
| 11-76 | 340.4 |
| 11-77 | 551.7 |
| 11-78 | 256.2 |
| 11-79 | 186.9 |
| 11-80 | 329.8 |
| 11-81 | 579.2 |
| 11-82 | 303 |
| 11-83 | 157.2 |
| 11-84 | 138.8 |
| 11-85 | 403.7 |
| 11-86 | 146.4 |
| 11-87 | 155.8 |
| 11-88 | 1651 |
| 11-89 | 3152 |
| 11-90 | 306.3 |
| 11-91 | 357.8 |
| 11-92 | 213 |
| 11-93 | 267.8 |
| 11-94 | 185 |
| 11-95 | 197.5 |
| 11-96 | 159.4 |
| 11-97 | 309.3 |
| 11-98 | 472.8 |
| 11-99 | 651.5 |
| 11-100 | 224.6 |
| 11-101 | 180.1 |
| 11-102 | 130.3 |
| 11-103 | 474.9 |
| 11-104 | 305.7 |
| 11-105 | 254.9 |
| 11-106 | 116.8 |
| 11-107 | 137.3 |
| 11-108 | 3694 |
| 11-109 | 1333 |
| 11-110 | 4875 |
| 11-111 | 285.1 |
| 11-112 | 184.4 |
| 11-113 | 249 |
| 11-114 | 257.3 |
| 11-115 | 378.9 |
| 11-116 | 156 |
| 11-117 | 350 |
| 11-118 | 233.2 |
| 11-119 | 132.4 |
| 11-120 | 168.2 |
| 11-121 | 59.98 |
| 11-122 | 319.5 |
| 11-123 | 285 |
| 11-124 | 166.6 |
| 11-125 | 531.3 |
| 11-126 | 347.2 |
| 11-127 | 323.2 |
| 11-128 | 274.6 |
| 11-129 | 960.5 |
| 11-130 | 120.7 |
| 11-131 | 309.5 |
| 11-132 | 172.5 |
| 11-133 | 174.3 |
| 11-134 | 604.6 |
| 11-135 | 169.1 |
| 11-136 | 161.7 |
| 11-137 | 688.2 |
| 11-138 | 392 |
| 11-139 | 272 |
| 11-140 | 119.1 |
| 11-141 | 274.1 |
| 11-142 | >10000 |
| 11-143 | 794.8 |
| 11-144 | 126.4 |
| 11-145 | >10000 |
| 11-146 | >10000 |
| 11-147 | 1038 |
| 11-148 | 1189 |
| 11-149 | >10000 |
| 11-150 | 342.5 |
| 11-151 | 346.9 |
| 11-152 | 4343 |
| 11-153 | 880 |
| 11-154 | 138.1 |
| 11-155 | 165.4 |
| 11-156 | 174.3 |
| 11-157 | 221.4 |
| 11-158 | 167.3 |
| 11-159 | 176.1 |
| 11-160 | 656.8 |
| 11-161 | 1049 |
| 11-162 | 756.6 |
| 11-163 | 3795 |
| 11-164 | 228 |
| 11-165 | 485.1 |
| 11-166 | 363.6 |
| 11-167 | 1178 |
| 11-168 | 240.7 |
| 11-169 | >10000 |
| 11-170 | 166.5 |
| 11-171 | 227 |
| 11-172 | 210.3 |
| 11-173 | 144.9 |
| 11-174 | 899.9 |
| 11-175 | 3005 |
| 11-176 | 722.5 |
| 11-177 | 1573 |
| 11-178 | 10000 |
| 11-179 | 1527 |
| 11-180 | >10000 |
| 11-181 | >10000 |
| 11-182 | 238.5 |
| 11-183 | 197.2 |
| 11-184 | 369.7 |
| 11-185 | 123.3 |
| 11-186 | 606 |
| 11-187 | 405.5 |
| 11-188 | 152.3 |
| 11-189 | 261.1 |
| 11-190 | 1106 |
| 11-191 | 1058 |
| 11-192 | 2442 |
| 11-193 | 1204 |
| 11-194 | 589.8 |
| 11-195 | 383.3 |
| 11-196 | 645.8 |
| 11-197 | 1294 |
| 11-198 | 232.2 |
| 11-199 | 498.3 |
| 11-200 | 128.8 |
| 11-201 | 117 |
| 11-202 | 303.5 |
| 11-203 | 356.3 |
| 11-204 | 1598 |
| 11-205 | 1562 |
| 11-206 | 131.8 |
| 11-207 | 341.9 |
| 11-208 | 468.6 |
| 11-209 | 439.4 |
| 11-210 | 360.6 |
| 11-211 | 520.5 |
| 11-212 | 1357 |
| 11-213 | 257.1 |
| 11-214 | 261.9 |
| 11-215 | 302.6 |
| 11-216 | >10000 |
| 11-217 | >10000 |
| 11-218 | 176.6 |
| 11-219 | 494.8 |
| 11-220 | 117.2 |
| 11-221 | 254.9 |
| 11-222 | 155.5 |
| 11-223 | 126.4 |
| 11-224 | 119.7 |
| 11-225 | 143.8 |
| 11-226 | 282.3 |
| 11-227 | 123.3 |
| 11-228 | 155 |
| 11-229 | |
| 11-230 | 200.6 |
| 11-231 | 192.4 |
| 11-232 | 3276 |
| 11-233 | 212.4 |
| 11-234 | 999.2 |
| 11-235 | 3605 |
| 11-236 | 678.6 |
| 11-237 | 1406 |
| 11-238 | 309.8 |
| 11-239 | 2971 |
| 11-240 | 573.4 |
| 11-241 | >10000 |
| 11-242 | >10000 |
| 11-243 | 7903 |
| 11-244 | 115.8 |
| 11-245 | 165.8 |
| 11-246 | 195.7 |
| 11-247 | 1542 |
| 11-248 | 2204 |
| 11-249 | 117.5 |
| 11-250 | 481.9 |
| 11-251 | 263.2 |
| 11-252 | 114 |
| 11-253 | 491.7 |
| 11-254 | 123.8 |
| 11-255 | 279.7 |
| 11-256 | 510.8 |
| 11-257 | 9522 |
| 11-258 | 119.3 |
| 11-259 | 398.3 |
| 11-260 | 368 |
| 11-261 | >10000 |
| 11-262 | 3068 |
| 11-263 | 1287 |
| 11-264 | 1175 |
| 11-265 | 247.9 |
| 11-266 | 396.2 |
| 11-267 | 235.8 |
| 11-268 | 362.3 |
| 11-269 | 110.3 |
| 11-270 | 226.9 |
| 11-271 | 118.1 |
| 11-272 | 141.4 |
| 11-273 | 3104 |
| 11-274 | >10000 |
| 11-275 | >10000 |
| 11-276 | >10000 |
| 11-277 | 4189 |
| 11-278 | 8351 |
| 11-279 | 192.3 |
| 11-280 | 382.7 |
| 11-281 | 427.5 |
| 11-282 | 1649 |
| 11-283 | 7173 |
| 11-284 | >10000 |
| 11-285 | >10000 |
| 11-286 | 283.8 |
| 11-287 | 306.5 |
| 11-288 | 720.9 |
| 11-289 | 1869 |
| 11-290 | 391.9 |
| 11-291 | 117.3 |
| 11-292 | 3951 |
| 11-293 | 167.6 |
| 11-294 | 169.9 |
| 11-295 | 503.2 |
| 11-296 | >10000 |
| 11-297 | 195.4 |
| 11-298 | 138.9 |
| 11-299 | 9254 |
| 11-300 | 3707 |
| 11-301 | 9151 |
| 11-302 | 1440 |
| 11-303 | 114.4 |
| 11-304 | 197.1 |
| 11-305 | 500.6 |
| 11-306 | 557.2 |
| 11-307 | 378.9 |
| 11-308 | 121.4 |
| 11-309 | 312.8 |
| 11-310 | 633.6 |
| 11-311 | 3973 |
| 11-312 | 5084 |
| 11-313 | >10000 |
| 11-314 | 146.5 |
| 11-315 | >10000 |
| 11-316 | >10000 |
| 11-317 | >10000 |
| 11-318 | 274.2 |
| 11-319 | 161.7 |
| 11-320 | 539.5 |
| 11-321 | 2801 |
| 11-322 | 710.6 |
| 11-323 | 156 |
| 11-324 | 3823 |
| 11-325 | 3079 |
| 11-326 | 2582 |
| 11-327 | 143 |
| 11-328 | 1911 |
| 11-329 | 1101 |
| 11-330 | >10000 |
| 11-331 | 300.6 |
| 11-332 | 480.7 |
| 11-333 | >10000 |
| 11-334 | 113.2 |
| 11-335 | 385.5 |
| 11-336 | 2744 |
| 11-337 | >10000 |
| 11-338 | 2421 |
| 11-339 | >10000 |
| 11-340 | >10000 |
| 11-341 | >10000 |
| 11-342 | 4473 |
| 11-343 | >10000 |
| 11-344 | 2154 |
| 11-345 | >10000 |
| 11-346 | >10000 |
| 11-347 | >10000 |
| 11-348 | 5485 |
| 11-349 | 332 |
| 11-350 | >10000 |
| 11-351 | 946.4 |
| 11-352 | 74.72 |
| 11-353 | 6295 |
| 11-354 | 702.9 |
| 11-355 | 879.9 |
| 11-356 | 532.3 |
| 11-357 | 195.1 |
| 11-358 | 1265 |
| 11-359 | 362.4 |
| 11-360 | 1089 |
| 11-361 | 276.5 |
| 11-362 | 1366 |
| 11-363 | 2890 |
| 11-364 | 5418 |
| 11-365 | 3274 |
| 11-366 | 144.1 |
| 11-367 | 600.4 |
| 11-368 | 855.7 |
| 11-369 | >10000 |
| 11-370 | >10000 |
| 11-371 | 9070 |
| 11-372 | >10000 |
| 11-373 | 7640 |
| 11-374 | >10000 |
| 11-375 | >10000 |
| 11-376 | 6003 |
| 11-377 | 3631 |
| 11-378 | >10000 |
| 11-379 | 3909 |
| 11-380 | >10000 |
| 11-381 | 5463 |
| 11-382 | 4323 |
| 11-383 | 4025 |
| 11-384 | >10000 |
| 11-385 | >10000 |
| 11-386 | >10000 |
| 11-387 | >10000 |
| 11-388 | >10000 |
| 11-389 | >10000 |
| 11-390 | >10000 |
| 11-391 | 9624 |
| 11-392 | 4044 |
| 11-393 | 1295 |
| 11-394 | >10000 |
| 11-395 | >10000 |
| 11-396 | 4379 |
| 11-397 | 3365 |
| 11-398 | >10000 |
| 11-399 | >10000 |
| 11-400 | 1047 |
| 11-401 | 3270 |
| 11-402 | 6865 |
| 11-403 | 1063 |
| 11-404 | >10000 |
| 11-405 | 8381 |
| 11-406 | 863.6 |
| 11-407 | >10000 |
| 11-408 | >10000 |
| 11-409 | >10000 |
| 11-410 | >10000 |
| 11-411 | >10000 |
| 11-412 | >10000 |
| 11-413 | 1650 |
| 11-414 | 1820 |
| 11-415 | 531.7 |
| 11-416 | 9092 |
| 11-417 | >10000 |
| 11-418 | >10000 |
| 11-419 | >10000 |
| 11-420 | 1309 |
| 11-421 | >10000 |
| 11-422 | >10000 |
| 11-423 | >10000 |
| 11-424 | |
| 11-425 | |
| 11-426 | |
| 11-427 | |
| 11-428 | |
| 11-429 | 3137 |
| 11-430 | 4671 |
| 11-431 | 403.4 |
| 11-432 | 121.8 |
| 11-433 | 3009 |
| 11-434 | 138.1 |
| 11-435 | 355.6 |
| 11-436 | 640.7 |
| 11-437 | |
| 11-438 | 187.9 |
| 11-439 | 241.4 |
| 11-440 | 252.4 |
| 11-441 | 408.4 |
| 11-442 | 871 |
| 11-443 | 130.3 |
| 11-444 | 233.9 |
| 11-445 | 286.6 |
| 11-446 | 300.1 |
| 11-447 | 644.7 |

TABLE E
Viability in MKN1 (PI3Kα E545K mutant)
cells by Exemplary Compounds of Formula (I)

| | MKN1 |
|---|---|
| | (E545K) |
| Example | Viability IC₅₀ |
| # | (nM) |
| 1 | >10000 |
| 2 | |
| 3-1 | >10000 |
| 3-2 | >10000 |
| 3-3 | >10000 |
| 3-4 | |
| 3-5 | >10000 |
| 3-6 | >10000 |
| 3-7 | >10000 |
| 3-8 | >10000 |
| 3-9 | >10000 |
| 3-10 | |
| 3-11 | >10000 |
| 3-12 | >10000 |
| 3-13 | >10000 |
| 3-14 | >10000 |
| 3-15 | >10000 |
| 3-16 | >10000 |
| 3-17 | >10000 |
| 3-18 | >10000 |
| 3-19 | >10000 |
| 3-20 | >10000 |
| 3-21 | |
| 3-22 | |
| 4-1 | >10000 |
| 4-2 | >10000 |
| 4-3 | 6139 |
| 4-4 | 4289 |
| 5-1 | >10000 |
| 5-2 | |
| 5-3 | >10000 |
| 5-4 | >10000 |
| 5-5 | >10000 |
| 5-6 | >10000 |
| 5-7 | 3284 |
| 5-8 | >10000 |
| 5-9 | >10000 |
| 5-9-1 | >10000 |
| 5-9-2 | >10000 |
| 5-10 | >10000 |
| 5-11 | >10000 |
| 6-1 | >10000 |
| 6-2 | >10000 |
| 7-1 | >10000 |
| 7-2 | |
| 8-1 | 9676 |
| 9-1 | >10000 |
| 9-2 | >10000 |
| 9-3 | 3781 |
| 9-4 | >10000 |

TABLE F
Viability in SKBR3 (PI3Kα wild type)
cells by Exemplary Compounds of Formula (I)

| | SKBR3 (WT) |
|---|---|
| | Viability IC₅₀ |
| Example # | (nM) |
| 1 | >10000 |
| 2 | >10000 |
| 3-1 | >10000 |
| 3-2 | >10000 |
| 3-3 | >10000 |
| 3-4 | |
| 3-5 | >10000 |
| 3-6 | >10000 |
| 3-7 | >10000 |
| 3-8 | >10000 |
| 3-9 | >10000 |
| 3-10 | >10000 |
| 3-11 | >10000 |
| 3-12 | >10000 |
| 3-13 | >10000 |
| 3-14 | >10000 |
| 3-15 | >10000 |
| 3-16 | 9302 |
| 3-17 | 9770 |
| 3-18 | >10000 |
| 3-19 | >10000 |
| 3-20 | >10000 |
| 3-21 | |
| 3-22 | 799 |
| 4-1 | >10000 |
| 4-2 | >10000 |
| 4-3 | 4843 |
| 4-4 | 3862 |
| 5-1 | >10000 |
| 5-2 | >10000 |
| 5-3 | >10000 |
| 5-4 | >10000 |
| 5-5 | >10000 |
| 5-6 | >10000 |
| 5-7 | 2949 |
| 5-8 | >10000 |
| 5-9 | >10000 |
| 5-10 | >10000 |
| 5-11 | >10000 |
| 5-9-1 | >10000 |
| 5-9-2 | >10000 |
| 6-1 | >10000 |
| 6-2 | >10000 |
| 7-1 | >10000 |
| 7-2 | >10000 |
| 8-1 | >10000 |
| 9-1 | >10000 |
| 9-2 | >10000 |
| 9-3 | 4771 |
| 9-4 | >10000 |
| 10-1 | 9892 |
| 10-2 | >10000 |
| 10-3 | >10000 |
| 10-4 | >10000 |
| 10-5 | 2189 |
| 10-6 | >10000 |
| 10-7 | >10000 |
| 10-8 | >10000 |
| 10-9 | >10000 |
| 10-10 | 7450 |
| 10-11 | 7774 |
| 10-12 | >10000 |
| 10-13 | 5891 |
| 10-14 | 8793 |
| 10-15 | >10000 |
| 10-16 | >10000 |
| 10-17 | >10000 |
| 10-18 | >10000 |
| 10-19 | 7641 |
| 10-20 | 7449 |
| 10-21 | 8074 |
| 10-22 | 5541 |
| 10-23 | 8972 |
| 10-24 | >10000 |
| 10-25 | >10000 |
| 10-26 | >10000 |
| 10-27 | >10000 |
| 10-28 | >10000 |
| 10-29 | >10000 |
| 10-30 | >10000 |
| 10-31 | >10000 |
| 10-32 | >10000 |
| 10-33 | >10000 |
| 10-34 | >10000 |
| 10-35 | 7952 |
| 10-36 | >10000 |
| 10-37 | >10000 |
| 10-38 | >10000 |
| 10-39 | >10000 |
| 10-40 | >10000 |
| 10-41 | >10000 |
| 10-42 | 9874 |
| 10-43 | >10000 |
| 10-44 | 5027 |
| 10-45 | >10000 |
| 10-46 | >10000 |
| 10-47 | 4541 |
| 10-48 | >10000 |
| 10-49 | >10000 |
| 10-50 | >10000 |
| 10-51 | >10000 |
| 10-52 | >10000 |
| 10-53 | >10000 |
| 10-54 | >10000 |
| 10-55 | 4139 |
| 10-56 | >10000 |
| 10-57 | >10000 |
| 10-58 | >10000 |
| 10-59 | >10000 |
| 10-60 | >10000 |
| 10-61 | >10000 |
| 10-62 | >10000 |
| 10-63 | >10000 |
| 10-64 | >10000 |
| 10-65 | >10000 |
| 10-66 | >10000 |
| 10-67 | >10000 |
| 10-68 | >10000 |
| 10-69 | >10000 |
| 10-70 | >10000 |
| 10-71 | >10000 |
| 10-72 | >10000 |
| 10-73 | >10000 |
| 10-74 | >10000 |
| 10-75 | >10000 |
| 10-76 | >10000 |
| 10-77 | >10000 |
| 10-78 | >10000 |
| 10-79 | >10000 |
| 10-80 | >10000 |
| 10-81 | >10000 |
| 10-82 | 7907 |
| 10-83 | >10000 |
| 10-84 | >10000 |
| 10-85 | >10000 |
| 10-86 | >10000 |
| 10-87 | >10000 |
| 10-88 | >10000 |
| 10-89 | >10000 |
| 10-90 | >10000 |
| 10-91 | >10000 |
| 10-92 | >10000 |
| 10-93 | >10000 |
| 10-94 | >10000 |
| 10-95 | >10000 |
| 10-96 | >10000 |
| 10-97 | >10000 |
| 10-98 | >10000 |
| 10-99 | 9941 |
| 10-100 | >10000 |
| 10-101 | >10000 |
| 10-102 | >10000 |
| 10-103 | 8758 |
| 10-104 | >10000 |
| 10-105 | >10000 |
| 10-106 | >10000 |
| 10-107 | >10000 |
| 10-108 | >10000 |
| 10-109 | 8832 |
| 10-110 | >10000 |
| 10-111 | >10000 |
| 10-112 | >10000 |
| 10-113 | >10000 |
| 10-114 | >10000 |
| 10-115 | >10000 |
| 10-116 | >10000 |
| 10-117 | 3719 |
| 10-118 | >10000 |
| 10-119 | >10000 |
| 10-120 | >10000 |
| 10-121 | >10000 |
| 10-122 | >10000 |
| 10-123 | >10000 |
| 10-124 | >10000 |
| 10-125 | >10000 |
| 10-126 | >10000 |
| 10-127 | >10000 |
| 10-128 | >10000 |
| 10-129 | >10000 |
| 10-130 | >10000 |
| 10-131 | >10000 |
| 10-132 | >10000 |
| 10-133 | >10000 |
| 10-134 | >10000 |
| 10-135 | >10000 |
| 10-136 | >10000 |
| 10-137 | >10000 |
| 10-138 | >10000 |
| 10-139 | >10000 |
| 10-140 | >10000 |
| 10-141 | >10000 |
| 10-142 | >10000 |
| 10-143 | >10000 |
| 10-144 | >10000 |
| 10-145 | >10000 |
| 10-146 | >10000 |
| 10-147 | >10000 |
| 10-148 | >10000 |
| 11-1 | |
| 11-2 | |
| 11-3 | |
| 11-4 | |
| 11-5 | |
| 11-6 | |
| 11-7 | |
| 11-8 | |
| 11-9 | |
| 11-10 | |
| 11-11 | |
| 11-12 | |
| 11-13 | >10000 |
| 11-14 | >10000 |
| 11-15 | >10000 |
| 11-16 | >10000 |
| 11-17 | >10000 |
| 11-18 | >10000 |
| 11-19 | >10000 |
| 11-20 | >10000 |
| 11-21 | >10000 |
| 11-22 | >10000 |
| 11-23 | >10000 |
| 11-24 | >10000 |
| 11-25 | |
| 11-26 | >10000 |
| 11-27 | >10000 |
| 11-28 | >10000 |
| 11-29 | >10000 |
| 11-30 | >10000 |
| 11-31 | >10000 |
| 11-32 | >10000 |
| 11-33 | >10000 |
| 11-34 | >10000 |
| 11-35 | >10000 |
| 11-36 | >10000 |
| 11-37 | >10000 |
| 11-38 | >10000 |
| 11-39 | >10000 |
| 11-40 | |
| 11-41 | |
| 11-42 | >10000 |
| 11-43 | >10000 |
| 11-44 | >10000 |
| 11-45 | >10000 |
| 11-46 | >10000 |
| 11-47 | >10000 |
| 11-48 | >10000 |
| 11-49 | >10000 |
| 11-50 | >10000 |
| 11-51 | >10000 |
| 11-52 | >10000 |
| 11-53 | >10000 |
| 11-54 | >10000 |
| 11-55 | >10000 |
| 11-56 | >10000 |
| 11-57 | >10000 |
| 11-58 | >10000 |
| 11-59 | >10000 |
| 11-60 | >10000 |
| 11-61 | >10000 |
| 11-62 | >10000 |
| 11-63 | >10000 |
| 11-64 | >10000 |
| 11-65 | >10000 |
| 11-66 | >10000 |
| 11-67 | >10000 |
| 11-68 | >10000 |
| 11-69 | >10000 |
| 11-70 | >10000 |
| 11-71 | >10000 |
| 11-72 | >10000 |
| 11-73 | >10000 |
| 11-74 | >10000 |
| 11-75 | >10000 |
| 11-76 | >10000 |
| 11-77 | >10000 |
| 11-78 | >10000 |
| 11-79 | 4549 |
| 11-80 | >10000 |
| 11-81 | >10000 |
| 11-82 | >10000 |
| 11-83 | >10000 |
| 11-84 | 952 |
| 11-85 | >10000 |
| 11-86 | >10000 |
| 11-87 | >10000 |
| 11-88 | >10000 |
| 11-89 | >10000 |
| 11-90 | >10000 |
| 11-91 | >10000 |
| 11-92 | >10000 |
| 11-93 | >10000 |
| 11-94 | >10000 |
| 11-95 | >10000 |
| 11-96 | >10000 |
| 11-97 | >10000 |
| 11-98 | >10000 |
| 11-99 | >10000 |
| 11-100 | >10000 |
| 11-101 | >10000 |
| 11-102 | >10000 |
| 11-103 | >10000 |
| 11-104 | >10000 |
| 11-105 | >10000 |
| 11-106 | >10000 |
| 11-107 | >10000 |
| 11-108 | >10000 |
| 11-109 | >10000 |
| 11-110 | >10000 |
| 11-111 | >10000 |
| 11-112 | >10000 |
| 11-113 | >10000 |
| 11-114 | >10000 |
| 11-115 | >10000 |
| 11-116 | >10000 |
| 11-117 | >10000 |
| 11-118 | >10000 |
| 11-119 | >10000 |
| 11-120 | >10000 |
| 11-121 | 5223 |
| 11-122 | >10000 |
| 11-123 | >10000 |
| 11-124 | >10000 |
| 11-125 | >10000 |
| 11-126 | >10000 |
| 11-127 | >10000 |
| 11-128 | >10000 |
| 11-129 | >10000 |
| 11-130 | >10000 |
| 11-131 | >10000 |
| 11-132 | >10000 |
| 11-133 | 9995 |
| 11-134 | >10000 |
| 11-135 | >10000 |
| 11-136 | >10000 |
| 11-137 | >10000 |
| 11-138 | >10000 |
| 11-139 | >10000 |
| 11-140 | >10000 |
| 11-141 | >10000 |
| 11-142 | >10000 |
| 11-143 | >10000 |
| 11-144 | >10000 |
| 11-145 | >10000 |
| 11-146 | >10000 |
| 11-147 | >10000 |
| 11-148 | >10000 |
| 11-149 | >10000 |
| 11-150 | >10000 |
| 11-151 | >10000 |
| 11-152 | >10000 |
| 11-153 | >10000 |
| 11-154 | >10000 |
| 11-155 | >10000 |
| 11-156 | >10000 |
| 11-157 | >10000 |
| 11-158 | >10000 |
| 11-159 | >10000 |
| 11-160 | >10000 |
| 11-161 | >10000 |
| 11-162 | >10000 |
| 11-163 | >10000 |
| 11-164 | >10000 |
| 11-165 | >10000 |
| 11-166 | >10000 |
| 11-167 | >10000 |
| 11-168 | >10000 |
| 11-169 | >10000 |
| 11-170 | >10000 |
| 11-171 | >10000 |
| 11-172 | >10000 |
| 11-173 | >10000 |
| 11-174 | >10000 |
| 11-175 | >10000 |
| 11-176 | >10000 |
| 11-177 | >10000 |
| 11-178 | >10000 |
| 11-179 | >10000 |
| 11-180 | >10000 |
| 11-181 | >10000 |
| 11-182 | >10000 |
| 11-183 | >10000 |
| 11-184 | >10000 |
| 11-185 | >10000 |
| 11-186 | >10000 |
| 11-187 | >10000 |
| 11-188 | >10000 |
| 11-189 | >10000 |
| 11-190 | >10000 |
| 11-191 | >10000 |
| 11-192 | >10000 |
| 11-193 | >10000 |
| 11-194 | >10000 |
| 11-195 | >10000 |
| 11-196 | >10000 |
| 11-197 | >10000 |
| 11-198 | >10000 |
| 11-199 | >10000 |
| 11-200 | >10000 |
| 11-201 | >10000 |
| 11-202 | >10000 |
| 11-203 | >10000 |
| 11-204 | >10000 |
| 11-205 | >10000 |
| 11-206 | >10000 |
| 11-207 | >10000 |
| 11-208 | >10000 |
| 11-209 | >10000 |
| 11-210 | >10000 |
| 11-211 | >10000 |
| 11-212 | >10000 |
| 11-213 | >10000 |
| 11-214 | >10000 |
| 11-215 | >10000 |
| 11-216 | >10000 |
| 11-217 | >10000 |
| 11-218 | >10000 |
| 11-219 | >10000 |
| 11-220 | >10000 |
| 11-221 | >10000 |
| 11-222 | >10000 |
| 11-223 | >10000 |
| 11-224 | >10000 |
| 11-225 | >10000 |
| 11-226 | >10000 |
| 11-227 | >10000 |
| 11-228 | >10000 |
| 11-229 | |
| 11-230 | >10000 |
| 11-231 | >10000 |
| 11-232 | >10000 |
| 11-233 | >10000 |
| 11-234 | >10000 |
| 11-235 | >10000 |
| 11-236 | >10000 |
| 11-237 | >10000 |
| 11-238 | >10000 |
| 11-239 | >10000 |
| 11-240 | 3229 |
| 11-241 | >10000 |
| 11-242 | >10000 |
| 11-243 | >10000 |
| 11-244 | >10000 |
| 11-245 | >10000 |
| 11-246 | >10000 |
| 11-247 | >10000 |
| 11-248 | 4311 |
| 11-249 | >10000 |
| 11-250 | >10000 |
| 11-251 | >10000 |
| 11-252 | >10000 |
| 11-253 | >10000 |
| 11-254 | >10000 |
| 11-255 | >10000 |
| 11-256 | >10000 |
| 11-257 | >10000 |
| 11-258 | >10000 |
| 11-259 | >10000 |
| 11-260 | >10000 |
| 11-261 | >10000 |
| 11-262 | >10000 |
| 11-263 | >10000 |
| 11-264 | >10000 |
| 11-265 | >10000 |
| 11-266 | >10000 |
| 11-267 | >10000 |
| 11-268 | >10000 |
| 11-269 | >10000 |
| 11-270 | >10000 |
| 11-271 | 7098 |
| 11-272 | >10000 |
| 11-273 | 7391 |
| 11-274 | >10000 |
| 11-275 | >10000 |
| 11-276 | >10000 |
| 11-277 | >10000 |
| 11-278 | >10000 |
| 11-279 | >10000 |
| 11-280 | >10000 |
| 11-281 | >10000 |
| 11-282 | >10000 |
| 11-283 | >10000 |
| 11-284 | >10000 |
| 11-285 | >10000 |
| 11-286 | >10000 |
| 11-287 | >10000 |
| 11-288 | >10000 |
| 11-289 | >10000 |
| 11-290 | >10000 |
| 11-291 | >10000 |
| 11-292 | >10000 |
| 11-293 | >10000 |
| 11-294 | >10000 |
| 11-295 | >10000 |
| 11-296 | >10000 |
| 11-297 | >10000 |
| 11-298 | >10000 |
| 11-299 | 9322 |
| 11-300 | >10000 |
| 11-301 | 8825 |
| 11-302 | 8968 |
| 11-303 | >10000 |
| 11-304 | >10000 |
| 11-305 | >10000 |
| 11-306 | >10000 |
| 11-307 | >10000 |
| 11-308 | >10000 |
| 11-309 | >10000 |
| 11-310 | >10000 |
| 11-311 | >10000 |
| 11-312 | >10000 |
| 11-313 | >10000 |
| 11-314 | >10000 |
| 11-315 | >10000 |
| 11-316 | >10000 |
| 11-317 | >10000 |
| 11-318 | >10000 |
| 11-319 | >10000 |
| 11-320 | >10000 |
| 11-321 | >10000 |
| 11-322 | >10000 |
| 11-323 | >10000 |
| 11-324 | >10000 |
| 11-325 | >10000 |
| 11-326 | 3960 |
| 11-327 | 9931 |
| 11-328 | 6817 |
| 11-329 | >10000 |
| 11-330 | >10000 |
| 11-331 | >10000 |
| 11-332 | 9806 |
| 11-333 | >10000 |
| 11-334 | 1272 |
| 11-335 | >10000 |
| 11-336 | >10000 |
| 11-337 | >10000 |
| 11-338 | >10000 |
| 11-339 | >10000 |
| 11-340 | >10000 |
| 11-341 | >10000 |
| 11-342 | >10000 |
| 11-343 | >10000 |
| 11-344 | >10000 |
| 11-345 | >10000 |
| 11-346 | >10000 |
| 11-347 | >10000 |
| 11-348 | >10000 |
| 11-349 | >10000 |
| 11-350 | >10000 |
| 11-351 | >10000 |
| 11-352 | >10000 |
| 11-353 | >10000 |
| 11-354 | >10000 |
| 11-355 | >10000 |
| 11-356 | 1199 |
| 11-357 | >10000 |
| 11-358 | 6207 |
| 11-359 | 8112 |
| 11-360 | 8258 |
| 11-361 | 8787 |
| 11-362 | >10000 |
| 11-363 | >10000 |
| 11-364 | >10000 |
| 11-365 | >10000 |
| 11-366 | 9620 |
| 11-367 | >10000 |
| 11-368 | |
| 11-369 | >10000 |
| 11-370 | >10000 |
| 11-371 | 3037 |
| 11-372 | >10000 |
| 11-373 | >10000 |
| 11-374 | >10000 |
| 11-375 | >10000 |
| 11-376 | >10000 |
| 11-377 | >10000 |
| 11-378 | >10000 |
| 11-379 | >10000 |
| 11-380 | >10000 |
| 11-381 | >10000 |
| 11-382 | >10000 |
| 11-383 | >10000 |
| 11-384 | |
| 11-385 | >10000 |
| 11-386 | >10000 |
| 11-387 | >10000 |
| 11-388 | >10000 |
| 11-389 | >10000 |
| 11-390 | >10000 |
| 11-391 | >10000 |
| 11-392 | >10000 |
| 11-393 | >10000 |
| 11-394 | >10000 |
| 11-395 | >10000 |
| 11-396 | >10000 |
| 11-397 | >10000 |
| 11-398 | >10000 |
| 11-399 | >10000 |
| 11-400 | >10000 |
| 11-401 | >10000 |
| 11-402 | >10000 |
| 11-403 | >10000 |
| 11-404 | >10000 |
| 11-405 | >10000 |
| 11-406 | >10000 |
| 11-407 | >10000 |
| 11-408 | >10000 |
| 11-409 | >10000 |
| 11-410 | >10000 |
| 11-411 | >10000 |
| 11-412 | >10000 |
| 11-413 | >10000 |
| 11-414 | >10000 |
| 11-415 | 890 |
| 11-416 | >10000 |
| 11-417 | >10000 |
| 11-418 | >10000 |
| 11-419 | >10000 |
| 11-420 | >10000 |
| 11-421 | >10000 |
| 11-422 | >10000 |
| 11-423 | >10000 |
| 11-424 | |
| 11-425 | |
| 11-426 | |
| 11-427 | |
| 11-428 | |
| 11-429 | >10000 |
| 11-430 | >10000 |
| 11-431 | >10000 |
| 11-432 | >10000 |
| 11-433 | >10000 |
| 11-434 | 8310 |
| 11-435 | >10000 |
| 11-436 | >10000 |
| 11-437 | |
| 11-438 | >10000 |
| 11-439 | >10000 |
| 11-440 | >10000 |
| 11-441 | >10000 |
| 11-442 | >10000 |
| 11-443 | >10000 |
| 11-444 | >10000 |
| 11-445 | >10000 |
| 11-446 | >10000 |
| 11-447 | >10000 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of formula (I): 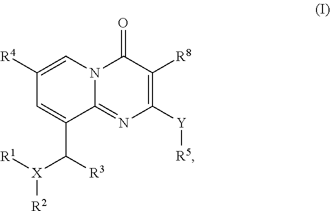
or a pharmaceutically acceptable salt or deuterated form thereof, wherein:
X is N, O, or S;
when X is N, R¹ is H, C₁-C₃ alkyl, or C₃-C₆ cycloalkyl;
when X is O or S, R¹ is absent;
R² is phenyl, 5-6 membered heteroaryl, 4-6 membered heterocyclyl, —C(O)—(C₂-C₄ alkyl), —C(O)—(C₂-C₄ alkenyl), —(C₂-C₄ alkyl)-C(O)OH or —(C₂-C₄ cycloalkyl)-C(O)OH, wherein each phenyl and heteroaryl is optionally substituted with 1-5 R⁷;
R³ is C₁-C₃ alkyl or C₃-C₈ cycloalkyl;
R⁴ is H, C₁-C₃ alkyl, C₃-C₆ cycloalkyl, cyano, or halo, wherein the C₁-C₃ alkyl is optionally substituted with 1-5 halo groups;
R⁵ is C₁-C₃ alkyl, C₂-C₆ alkenyl, 6-10 membered aryl, 5-10 membered heteroaryl, C₃-C₆ cycloalkyl, or 3-9 membered heterocyclyl, wherein each alkyl, alkenyl, aryl, heteroaryl, cycloalkyl, and heterocyclyl is optionally substituted with 1-5 R⁶, and wherein heterocyclyl is optionally unsaturated;
R⁸ is R^{A}, halogen, or cyano,
Y is a bond, NR¹°, S or O;
wherein:
each R⁶ is independently —R^{A}, —OR^{A}, —C(O)OR^{A}, oxo, cyano, halo, (C₁-C₃ alkyl)-OR^{A}, —C(O)N(R^{A})₂, —C(O) R¹¹, —N(R^{A})₂, —N(R^{A}) C(O) R^{A}, —SO₂R^{A}, —R^{C}, or two geminal R$ together with the carbon to which they are attached form a spirocyclic C₃-C₆ cycloalkyl or 3-12 membered heterocyclyl, or two vicinal R⁸ together with the carbon to which they are attached form a fused C₁₀-C₁₄ aryl, C₈-C₁₂ cycloalkyl or C₃-C₁₂ heterocyclyl;
wherein each spirocyclic group and fused group is optionally substituted with R^{B}, —OR^{B}, —C(O)OR^{B}, (C₁-C₃ alkyl)-OR^{B}, —C(O)N(R^{B})₂, oxo, cyano, halo,
wherein each R^{B} is independently H, C₁-C₆ alkyl, C₃-C₆ cycloalkyl or C₃-C₆ cycloalkyl(C₁-C₆) alkyl;
R^{C} represents C₃-C₆ cycloalkyl, C₃-C₁₂ heterocyclyl, C₃-C₁₂ heterocyclyl(C₁-C₆) alkyl, phenyl, phenyl(C₁-C₂) alkyl, 5-10 membered heteroaryl, or 5-10 membered heteroaryl(C₁-C₂) alkyl, wherein each cycloalkyl, heterocycyclyl, phenyl and heteroaryl is optionally substituted with 1-5 of R⁹,
each R⁷ is independently cyano, halo, oxo, C₁-C₄ alkyl, —OR^{A}, —C(O)OR^{A}, (C₁-C₃ alkyl)-C(O)OR^{A}, (C₁-C₃ alkyl)-OR^{A}, —C(O)N(R^{A})₂, —P(O) (OH) R^{A} or two geminal R⁷ together with the carbon to which they are attached form a C₃-C₆ cycloalkyl or 3-9 membered heterocyclyl;
each R⁹ is independently hydroxy, halogen, cyano, C₁-C₆ alkyl, C₁-C₆ alkoxy, halo(C₁-C₆) alkyl, or —SO₂N(R¹⁰)₂;
each R¹° is independently hydrogen or C₁-C₆ alkyl;
R¹¹ is C₁-C₆ alkyl, C₃-C₆ cycloalkyl(C₁-C₆) alkyl, C₃-C₆ cycloalkyl, or phenyl, each of which is optionally substituted with 1-5 R⁷;
each R^{A} is independently H, C₁-C₆ alkyl, C₃-C₆ cycloalkyl, C₃-C₆ cycloalkyl(C₁-C₆) alkyl or two geminal R^{A} together with the carbon to which they are attached form a C₃-C₈ cycloalkyl or 3-9 membered heterocyclyl; and
wherein, unless otherwise indicated, each alkyl and cycloalkyl group is unsubstituted, substituted with 1-5 halo groups, perfluorinated, or is substituted by 1-3 groups selected from methyl, oxo, and carboxy; and
provided R⁵ is not unsubstituted morpholinyl when Y is a bond.

2. The compound of claim 1, wherein X is N.

3. The compound of claim 1 or claim 2, wherein R¹ is H or CH₃.

4. The compound of any of claims 1-3, wherein R² is phenyl, pyridinyl, —C(O)—CH═CH₂, or —C(O)—CH₂-C₁.

5. The compound of any of claims 1-4, wherein R³ is C₁-C₃ alkyl, wherein the alkyl group is unsubstituted, substituted with 1-5 halo groups, or perfluorinated.

6. The compound of any of claims 1-5, wherein Y is a bond.

7. The compound of any of claims 1-6, wherein R⁵ is a 6-10 membered aryl, C₃-C₆ cycloalkyl or 3-9 membered heterocyclyl, and each is optionally substituted with 1-5 R⁶.

8. The compound of any of claims 1-7, wherein R⁵ is cyclopropyl, phenyl, piperidinyl, or isoindolinyl and each is optionally substituted with 1-5 R⁶.

9. The compound of any of claims 1-8, wherein R⁸ is hydrogen, methyl, ethyl, or cyano.

10. The compound of any of claims 1-9, wherein the compound is of formula (IIa), (IIb) or (IIIc): 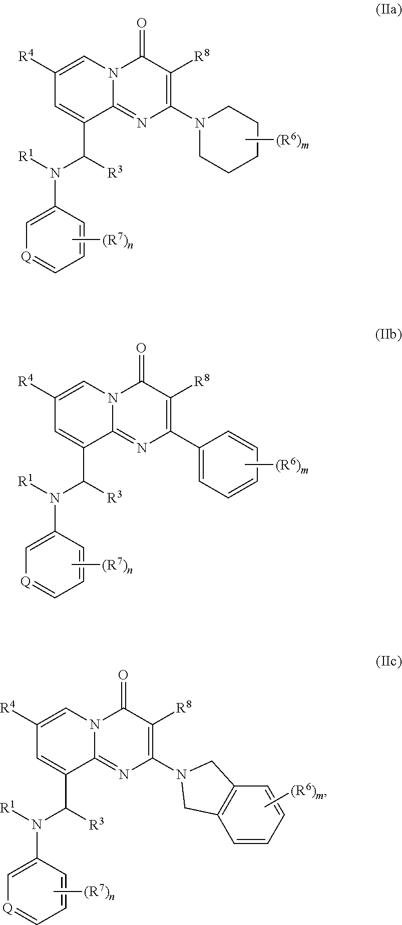
or a pharmaceutically acceptable salt thereof, wherein:
Q is N or CH;
R¹ is H or CH₃;
R³ is C₁-C₃ alkyl;
R⁴ is H, C₁-C₃ alkyl, C₃-C₈ cycloalkyl, cyano, or halo, wherein the C₁-C₃ alkyl is optionally substituted with 1-5 halo groups;
each R⁶ is independently —R^{A}, —OR^{A}, —C(O)OR^{A}, (C₁-C₃ alkyl)-OR^{A}, —C(O)N(R^{A})₂, oxo, cyano, halo, or two geminal R& together with the carbon to which they are attached form a C₃-C₈ cycloalkyl or 3-9 membered heterocyclyl, or two vicinal R⁶ together with the carbon to which they are attached form a fused C₁₀-C₁₄ aryl or C₈-C₁₂ cycloalkyl;
each m is 0, 1, 2, 3, or 4;
each R⁷ is independently C₁-C₄ alkyl; —OR^{A}, —C(O)OR^{A}, (C₁-C₃ alkyl)-OR^{A}, —C(O)N(R^{A})₂, cyano, halo, or two geminal R⁷ together with the carbon to which they are attached form a C₃-C₈ cycloalkyl or 3-9 membered heterocyclyl;
each n is 0, 1, 2, 3, or 4;
R⁸ is methyl, ethyl or cyano;
each R^{A} is independently H, C₁-C₃ alkyl, C₃-C₆ cycloalkyl, or two geminal R^{A} together with the carbon to which they are attached form a C₃-C₈ cycloalkyl or 3-9 membered heterocyclyl; and
wherein each alkyl and cycloalkyl group is unsubstituted, substituted with 1-5 halo groups, perfluorinated, or is substituted by 1-3 groups selected from methyl, oxo, or carboxy.

11. The compound of claim 10, wherein Q is CH.

12. The compound of claim 10 or claim 11, wherein R¹ is H.

13. The compound of any of claims 10-12, wherein R³ is unsubstituted methyl.

14. The compound of any of claims 10-13, wherein R⁴ is H, methyl, ethyl, or cyclopropyl, wherein each is unsubstituted.

15. The compound of any of claims 10-14, wherein R⁴ is H or unsubstituted methyl or difluoromethyl.

16. The compound of any of claims 10-15, wherein m is 2 and each R⁸ is unsubstituted methyl.

17. The compound of any of claims 10-16, wherein n is 1 and R⁷ is —COOH.

18. The compound of any of claims 10-17, wherein R⁸ is H.

19. The compound of claim 1, which is:
7-methyl-9-(1-(phenylamino)ethyl)-2-(piperidin-1-yl)-4H-pyrido[1,2-a]pyrimidin-4-one;
2-((1-(7-methyl-4-oxo-2-(piperidin-1-yl)-4H-pyrido[1,2-a]pyrimidin-9-yl)ethyl)amino)benzoic acid;
(R)-2-((1-(7-methyl-4-oxo-2-(piperidin-1-yl)-4H-pyrido[1,2-a]pyrimidin-9-yl)ethyl)amino)benzoic acid;
(S)-2-((1-(7-methyl-4-oxo-2-(piperidin-1-yl)-4H-pyrido[1,2-a]pyrimidin-9-yl)ethyl)amino)benzoic acid;
2-methoxy-6-((1-(7-methyl-4-oxo-2-(piperidin-1-yl)-4H-pyrido[1,2-a]pyrimidin-9-yl)ethyl)amino)benzoic acid;
2-hydroxy-6-((1-(7-methyl-4-oxo-2-(piperidin-1-yl)-4H-pyrido[1,2-a]pyrimidin-9-yl)ethyl)amino)benzoic acid;
5-methoxy-2-((1-(7-methyl-4-oxo-2-(piperidin-1-yl)-4H-pyrido[1,2-a]pyrimidin-9-yl)ethyl)amino)benzoic acid;
5-hydroxy-2-((1-(7-methyl-4-oxo-2-(piperidin-1-yl)-4H-pyrido[1,2-a]pyrimidin-9-yl)ethyl)amino)benzoic acid;
4-methoxy-2-((1-(7-methyl-4-oxo-2-(piperidin-1-yl)-4H-pyrido[1,2-a]pyrimidin-9-yl)ethyl)amino)benzoic acid;
4-hydroxy-2-((1-(7-methyl-4-oxo-2-(piperidin-1-yl)-4H-pyrido[1,2-a]pyrimidin-9-yl)ethyl)amino)benzoic acid;
2-(1-(7-methyl-4-oxo-2-(piperidin-1-yl)-4H-pyrido[1,2-a]pyrimidin-9-yl)ethoxy)benzoic acid;
2-((1-(7-methyl-4-oxo-2-(piperidin-1-yl)-4H-pyrido[1,2-a]pyrimidin-9-yl)ethyl) thio) benzoic acid;
2-((1-(7-methyl-4-oxo-2-(piperidin-1-yl)-4H-pyrido[1,2-a]pyrimidin-9-yl)ethyl)amino) benzonitrile;
methyl 2-((1-(7-methyl-4-oxo-2-(piperidin-1-yl)-4H-pyrido[1,2-a]pyrimidin-9-yl)ethyl)amino) benzoate;
2-((1-(7-methyl-4-oxo-2-(piperidin-1-yl)-4H-pyrido[1,2-a]pyrimidin-9-yl)ethyl)amino)benzamide;
N-methyl-2-((1-(7-methyl-4-oxo-2-(piperidin-1-yl)-4H-pyrido[1,2-a]pyrimidin-9-yl)ethyl)amino)benzamide;
N, N-dimethyl-2-((1-(7-methyl-4-oxo-2-(piperidin-1-yl)-4H-pyrido[1,2-a]pyrimidin-9-yl)ethyl)amino)benzamide;
9-(1-((2-hydroxyphenyl)amino)ethyl)-7-methyl-2-(piperidin-1-yl)-4H-pyrido[1,2-a]pyrimidin-4-one;
9-(1-((1H-indazol-7-yl)amino)ethyl)-7-methyl-2-(piperidin-1-yl)-4H-pyrido[1,2-a]pyrimidin-4-one;
7-methyl-2-(piperidin-1-yl)-9-(1-((2-(2,2,2-trifluoro-1-hydroxyethyl)phenyl)amino)ethyl)-4H-pyrido[1,2-a]pyrimidin-4-one;
6-chloro-3-((1-(7-methyl-4-oxo-2-(piperidin-1-yl)-4H-pyrido[1,2-a]pyrimidin-9-yl)ethyl)amino) picolinic acid;
9-(1-((2-(1,1-difluoro-2-hydroxyethyl)phenyl)amino)ethyl)-7-methyl-2-(piperidin-1-yl)-4H-pyrido[1,2-a]pyrimidin-4-one;
2-(methyl (1-(7-methyl-4-oxo-2-(piperidin-1-yl)-4H-pyrido[1,2-a]pyrimidin-9-yl)ethyl)amino)benzoic acid;
2-((1-(7-methyl-4-oxo-2-(6-azaspiro[2.5]octan-6-yl)-4H-pyrido[1,2-a]pyrimidin-9-yl)ethyl)amino)benzoic acid;
N-(1-(7-methyl-4-oxo-2-(piperidin-1-yl)-4H-pyrido[1,2-a]pyrimidin-9-yl)ethyl)acrylamide;
N-methyl-N-(1-(7-methyl-4-oxo-2-(piperidin-1-yl)-4H-pyrido[1,2-a]pyrimidin-9-yl)ethyl)acrylamide;
2-chloro-N-(1-(7-methyl-4-oxo-2-(piperidin-1-yl)-4H-pyrido[1,2-a]pyrimidin-9-yl)ethyl)acetamide;
2-chloro-N-methyl-N-(1-(7-methyl-4-oxo-2-(piperidin-1-yl)-4H-pyrido[1,2-a]pyrimidin-9-yl)ethyl)acetamide;
(R)-2-((1-(2-(4,4-dimethylpiperidin-1-yl)-3,7-dimethyl-4-oxo-4H-pyrido[1,2-a]pyrimidin-9-yl)ethyl)amino)benzoic acid;
(S)-2-((1-(2-(4,4-dimethylpiperidin-1-yl)-3,7-dimethyl-4-oxo-4H-pyrido[1,2-a]pyrimidin-9-yl)ethyl)amino)benzoic acid;
2-((1-(7-cyclopropyl-4-oxo-2-(piperidin-1-yl)-4H-pyrido[1,2-a]pyrimidin-9-yl)ethyl)amino)benzoic acid;
(S)-2-((1-(2-(4,4-dimethylpiperidin-1-yl)-7-methyl-4-oxo-4H-pyrido[1,2-a]pyrimidin-9-yl)ethyl)amino)benzoic acid;
(R)-2-((1-(2-(4,4-dimethylpiperidin-1-yl)-7-methyl-4-oxo-4H-pyrido[1,2-a]pyrimidin-9-yl)ethyl)amino)benzoic acid;
2-((1-(3-ethyl-7-methyl-4-oxo-2-(piperidin-1-yl)-4H-pyrido[1,2-a]pyrimidin-9-yl)ethyl)amino)benzoic acid;
2-((1-(7-(difluoromethyl)-4-oxo-2-(piperidin-1-yl)-4H-pyrido[1,2-a]pyrimidin-9-yl)ethyl)amino)benzoic acid;
2-((1-(7-ethyl-4-oxo-2-(piperidin-1-yl)-4H-pyrido[1,2-a]pyrimidin-9-yl)ethyl)amino)benzoic acid;
2-((1-(2-(isoindolin-2-yl)-3,7-dimethyl-4-oxo-4H-pyrido[1,2-a]pyrimidin-9-yl)ethyl)amino)benzoic acid;
(S)-2-((1-(2-(isoindolin-2-yl)-3,7-dimethyl-4-oxo-4H-pyrido[1,2-a]pyrimidin-9-yl)ethyl)amino)benzoic acid;
(R)-2-((1-(2-(isoindolin-2-yl)-3,7-dimethyl-4-oxo-4H-pyrido[1,2-a]pyrimidin-9-yl)ethyl)amino)benzoic acid;
2-((1-(2-(4,4-dimethylpiperidin-1-yl)-3-methyl-4-oxo-4H-pyrido[1,2-a]pyrimidin-9-yl)ethyl)amino)benzoic acid;
2-((1-(3-methyl-4-oxo-2-(piperidin-1-yl)-4H-pyrido[1,2-a]pyrimidin-9-yl)ethyl)amino)benzoic acid;
2-((1-(2,7-dimethyl-4-oxo-4H-pyrido[1,2-a]pyrimidin-9-yl)ethyl)amino)benzoic acid;
2-((1-(2-cyclopropyl-7-methyl-4-oxo-4H-pyrido[1,2-a]pyrimidin-9-yl)ethyl)amino)benzoic acid;
2-((1-(7-methyl-4-oxo-2-phenyl-4H-pyrido[1,2-a]pyrimidin-9-yl)ethyl)amino)benzoic acid;
2-((1-(7-methyl-4-oxo-2-(p-tolyl)-4H-pyrido[1,2-a]pyrimidin-9-yl)ethyl)amino)benzoic acid;
2-((1-(3-cyano-7-methyl-4-oxo-2-(piperidin-1-yl)-4H-pyrido[1,2-a]pyrimidin-9-yl)ethyl)amino)benzoic acid;
2-((1-(7-methyl-4-oxo-2-(tetrahydro-2H-pyran-4-yl)-4H-pyrido[1,2-a]pyrimidin-9-yl)ethyl)amino)benzoic acid;
2-((1-(2-(4,4-dimethylpiperidin-1-yl)-4-oxo-4H-pyrido[1,2-a]pyrimidin-9-yl)ethyl)amino)benzoic acid;
2-((1-(2-(benzyloxy)-7-methyl-4-oxo-4H-pyrido[1,2-a]pyrimidin-9-yl)ethyl)amino)benzoic acid;
2-((1-(2-(isoindolin-2-yl)-4-oxo-4H-pyrido[1,2-a]pyrimidin-9-yl)ethyl)amino)benzoic acid;
2-((1-(2-(3-cyanophenyl)-7-methyl-4-oxo-4H-pyrido[1,2-a]pyrimidin-9-yl)ethyl)amino)benzoic acid;
2-((1-(2-(4-cyanophenyl)-7-methyl-4-oxo-4H-pyrido[1,2-a]pyrimidin-9-yl)ethyl)amino)benzoic acid;
2-((1-(2-(4-chlorophenyl)-7-methyl-4-oxo-4H-pyrido[1,2-a]pyrimidin-9-yl)ethyl)amino)benzoic acid;
2-((1-(7-methyl-4-oxo-2-(p-tolyl)-4H-pyrido[1,2-a]pyrimidin-9-yl)ethyl)amino)benzoic acid;
2-((1-(7-methyl-4-oxo-2-(o-tolyl)-4H-pyrido[1,2-a]pyrimidin-9-yl)ethyl)amino)benzoic acid;
2-((1-(2-(2-methoxyphenyl)-7-methyl-4-oxo-4H-pyrido[1,2-a]pyrimidin-9-yl)ethyl)amino)benzoic acid;
2-((1-(2-(3-methoxyphenyl)-7-methyl-4-oxo-4H-pyrido[1,2-a]pyrimidin-9-yl)ethyl)amino)benzoic acid;
2-((1-(2-(4-methoxyphenyl)-7-methyl-4-oxo-4H-pyrido[1,2-a]pyrimidin-9-yl)ethyl)amino)benzoic acid;
2-((1-(2-(4-fluorophenyl)-7-methyl-4-oxo-4H-pyrido[1,2-a]pyrimidin-9-yl)ethyl)amino)benzoic acid;
2-((1-(3-methyl-4-oxo-2-(piperidin-1-yl)-4H-pyrido[1,2-a]pyrimidin-9-yl)ethyl)amino)benzoic acid;
3-methyl-3-((1-(7-methyl-4-oxo-2-(piperidin-1-yl)-4H-pyrido[1,2-a]pyrimidin-9-yl)ethyl)amino) butanoic acid;
3-((1-(7-methyl-4-oxo-2-(piperidin-1-yl)-4H-pyrido[1,2-a]pyrimidin-9-yl)ethyl)amino) propanoic acid;
3-((1-(7-methyl-4-oxo-2-(piperidin-1-yl)-4H-pyrido[1,2-a]pyrimidin-9-yl)ethyl)amino) propanoic acid;
3-((1-(7-methyl-4-oxo-2-(piperidin-1-yl)-4H-pyrido[1,2-a]pyrimidin-9-yl)ethyl)amino)cyclobutane-1-carboxylic acid;
2-((1-(7-methyl-2-(4-methyl-3-oxopiperazin-1-yl)-4-oxo-4H-pyrido[1,2-a]pyrimidin-9-yl)ethyl)amino)benzoic acid;
2-((1-(7-methyl-4-oxo-2-(tetrahydro-2H-pyran-4-yl)-4H-pyrido[1,2-a]pyrimidin-9-yl)ethyl)amino)benzoic acid;
2-((1-(7-methyl-2-(1-methyl-6-oxo-1,6-dihydropyridin-3-yl)-4-oxo-4H-pyrido[1,2-a]pyrimidin-9-yl)ethyl)amino)benzoic acid;
2-((1-(7-methyl-2-(1-methyl-1,2,3,4,5,7-hexahydro-6H-pyrazolo[3,4-c]pyridin-6-yl)-4-oxo-4H-pyrido[1,2-a]pyrimidin-9-yl)ethyl)amino) benzoic acid;
2-((1-(2-(1-acetylpiperidin-4-yl)-7-methyl-4-oxo-4H-pyrido[1,2-a]pyrimidin-9-yl)ethyl)amino)benzoic acid;
2-((1-(7-methyl-2-(1-methyl-2-oxopiperidin-4-yl)-4-oxo-4H-pyrido[1,2-a]pyrimidin-9-yl)ethyl)amino)benzoic acid;
2-((1-(7-methyl-4-oxo-2-(1H-pyrazol-4-yl)-4H-pyrido[1,2-a]pyrimidin-9-yl)ethyl)amino)benzoic acid;
2-((1-(7-methyl-2-(1-methyl-1H-pyrazol-4-yl)-4-oxo-4H-pyrido[1,2-a]pyrimidin-9-yl)ethyl)amino)benzoic acid;
2-((1-(7-methyl-2-(1-methyl-1H-pyrazol-3-yl)-4-oxo-4H-pyrido[1,2-a]pyrimidin-9-yl)ethyl)amino)benzoic acid;
2-((1-(3,7-dimethyl-4-oxo-2-phenyl-4H-pyrido[1,2-a]pyrimidin-9-yl)ethyl)amino)benzoic acid;
2-((1-(7-methyl-4-oxo-2-(pyridin-2-yl)-4H-pyrido[1,2-a]pyrimidin-9-yl)ethyl)amino)benzoic acid;
2-((1-(7-methyl-4-oxo-2-(pyridin-3-yl)-4H-pyrido[1,2-a]pyrimidin-9-yl)ethyl)amino)benzoic acid;
2-((1-(7-methyl-4-oxo-2-(pyridin-4-yl)-4H-pyrido[1,2-a]pyrimidin-9-yl)ethyl)amino)benzoic acid; 2-((1-(2-(2-cyanophenyl)-7-methyl-4-oxo-4H-pyrido[1,2-a]pyrimidin-9-yl)ethyl)amino)benzoic acid;
2-((1-(7-methyl-4-oxo-2-((tetrahydrofuran-3-yl)oxy)-4H-pyrido[1,2-a]pyrimidin-9-yl)ethyl)amino)benzoic acid;
(S)-2-((1-(2-(isoindolin-2-yl)-3,7-dimethyl-4-oxo-4H-pyrido[1,2-a]pyrimidin-9-yl)ethyl)amino)benzoic acid;
(R)-2-((1-(2-(isoindolin-2-yl)-3,7-dimethyl-4-oxo-4H-pyrido[1,2-a]pyrimidin-9-yl)ethyl)amino)benzoic acid;
2-((1-(2-(4,4-dimethylpiperidin-1-yl)-3-methyl-4-oxo-4H-pyrido[1,2-a]pyrimidin-9-yl)ethyl)amino)benzoic acid; or
2-((1-(3-methyl-4-oxo-2-(piperidin-1-yl)-4H-pyrido[1,2-a]pyrimidin-9-yl)ethyl)amino)benzoic acid;
(R)-2-((1-(2-(4-(4-cyanophenyl) piperazin-1-yl)-3,7-dimethyl-4-oxo-4H-pyrido[1,2-a]pyrimidin-9-yl)ethyl)amino)benzoic acid;
2-(((1R)-1-(3,7-dimethyl-2-(3-(1-methyl-1H-pyrazol-5-yl)-8-azabicyclo[3.2.1]octan-8-yl)-4-oxo-4H-pyrido[1,2-a]pyrimidin-9-yl)ethyl)amino)benzoic acid;
(R)-2-((1-(3,7-dimethyl-4-oxo-2-(2-oxo-[1,4′-bipiperidin]-1′-yl)-4H-pyrido[1,2-a]pyrimidin-9-yl)ethyl)amino)benzoic acid;
(R)-2-((1-(2-(4-(ethoxymethyl) piperidin-1-yl)-3,7-dimethyl-4-oxo-4H-pyrido[1,2-a]pyrimidin-9-yl)ethyl)amino)benzoic acid;
(R)-3-((1-(3-cyano-2-(isoindolin-2-yl)-7-methyl-4-oxo-4H-pyrido[1,2-a]pyrimidin-9-yl)ethyl)amino)-6-methylpicolinic acid;
(R)-2-((1-(2-(4-(2-cyclopropylacetyl) piperazin-1-yl)-3,7-dimethyl-4-oxo-4H-pyrido[1,2-a]pyrimidin-9-yl)ethyl)amino)benzoic acid;
(R)-2-((1-(2-(3,4-dihydroisoquinolin-2(1H)-yl)-3,7-dimethyl-4-oxo-4H-pyrido[1,2-a]pyrimidin-9-yl)ethyl)amino)benzoic acid;
2-(((1R)-1-(2-(1-hydroxy-6-azaspiro[3.5]nonan-6-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(3-(pyridin-4-yl) pyrrolidin-1-yl)-4H-pyrido[1,2-a]pyrimidin-9-yl)ethyl)amino)benzoic acid;
(R)-2-((1-(3-cyano-7-ethyl-2-(isoindolin-2-yl)-4-oxo-4H-pyrido[1,2-a]pyrimidin-9-yl)ethyl)amino)benzoic acid;
(R)-2-((1-(7-chloro-3-cyano-2-(isoindolin-2-yl)-4-oxo-4H-pyrido[1,2-a]pyrimidin-9-yl)ethyl)amino)benzoic acid;
(R)-2-((1-(3-bromo-2-(4,4-difluoropiperidin-1-yl)-7-methyl-4-oxo-4H-pyrido[1,2-a]pyrimidin-9-yl)ethyl)amino)benzoic acid;
(R)-2-((1-(3,7-dimethyl-2-(4-(1-methyl-3-(trifluoromethyl)-1H-pyrazol-4-yl) piperazin-1-yl)-4-oxo-4H-pyrido[1,2-a]pyrimidin-9-yl)ethyl)amino)benzoic acid;
(R)-6-chloro-3-((1-(3-cyano-2-(isoindolin-2-yl)-7-methyl-4-oxo-4H-pyrido[1,2-a]pyrimidin-9-yl)ethyl)amino) picolinic acid;
(R)-2-((1-(3-cyano-2-((cyclopropylmethyl)amino)-7-methyl-4-oxo-4H-pyrido[1,2-a]pyrimidin-9-yl)ethyl)amino)benzoic acid;
(R)-2-((1-(3,7-dimethyl-2-(4-(4-methyl-1H-pyrazol-1-yl) piperidin-1-yl)-4-oxo-4H-pyrido[1,2-a]pyrimidin-9-yl)ethyl)amino)benzoic acid;
(R)-3-((1-(3-cyano-2-(4,4-difluoropiperidin-1-yl)-7-methyl-4-oxo-4H-pyrido[1,2-a]pyrimidin-9-yl)ethyl)amino)-6-methylpicolinic acid;
(R)-2-((1-(2-(3,3-difluoropiperidin-1-yl)-3,7-dimethyl-4-oxo-4H-pyrido[1,2-a]pyrimidin-9-yl)ethyl)amino)benzoic acid;
(R)-2-((1-(2-(4-(8-cyanoisoquinolin-5-yl) piperazin-1-yl)-3,7-dimethyl-4-oxo-4H-pyrido[1,2-a]pyrimidin-9-yl)ethyl)amino)benzoic acid;
2-(((1R)-1-(2-(4-(4-cyanophenyl)-2-methylpiperazin-1-yl)-3,7-dimethyl-4-oxo-4H-pyrido[1,2-a]pyrimidin-9-yl)ethyl)amino)benzoic acid;
2-(((R)-1-(2-((S)-4-(4-cyanophenyl)-3-methylpiperazin-1-yl)-3,7-dimethyl-4-oxo-4H-pyrido[1,2-a]pyrimidin-9-yl)ethyl)amino)benzoic acid;
2-(((R)-1-(2-((R)-4-(4-cyanophenyl)-2-methylpiperazin-1-yl)-3,7-dimethyl-4-oxo-4H-pyrido[1,2-a]pyrimidin-9-yl)ethyl)amino)benzoic acid;
(R)-2-((1-(3-cyano-2-(4,4-difluoropiperidin-1-yl)-7-methyl-4-oxo-4H-pyrido[1,2-a]pyrimidin-9-yl)ethyl)amino)benzoic acid;
2-(((R)-1-(2-((R)-4-(4-cyanophenyl)-3-methylpiperazin-1-yl)-3,7-dimethyl-4-oxo-4H-pyrido[1,2-a]pyrimidin-9-yl)ethyl)amino)benzoic acid;
(R)-2-((1-(3-cyano-2-(4,4-difluoropiperidin-1-yl)-7-fluoro-4-oxo-4H-pyrido[1,2-a]pyrimidin-9-yl)ethyl)amino)benzoic acid;
(R)-2-((1-(7-chloro-3-cyano-2-(4,4-difluoropiperidin-1-yl)-4-oxo-4H-pyrido[1,2-a]pyrimidin-9-yl)ethyl)amino)benzoic acid;
(R)-2-((1-(3-cyano-2-(3,3-difluoroazetidin-1-yl)-7-methyl-4-oxo-4H-pyrido[1,2-a]pyrimidin-9-yl)ethyl)amino)benzoic acid;
2-(((R)-1-(2-((R)-3,3-difluoro-4-methoxypiperidin-1-yl)-3,7-dimethyl-4-oxo-4H-pyrido[1,2-a]pyrimidin-9-yl)ethyl)amino)benzoic acid;
(R)-6-fluoro-3-((1-(2-(isoindolin-2-yl)-3,7-dimethyl-4-oxo-4H-pyrido[1,2-a]pyrimidin-9-yl)ethyl)amino) picolinic acid;
(R)-6-chloro-3-((1-(2-(isoindolin-2-yl)-3,7-dimethyl-4-oxo-4H-pyrido[1,2-a]pyrimidin-9-yl)ethyl)amino) picolinic acid;
2-(((R)-1-(3-cyano-2-((S)-3,3-difluoro-2-methylpyrrolidin-1-yl)-7-methyl-4-oxo-4H-pyrido[1,2-a]pyrimidin-9-yl)ethyl)amino)benzoic acid;
2-(((R)-1-(3-cyano-2-((R)-3,3-difluoro-2-methylpyrrolidin-1-yl)-7-methyl-4-oxo-4H-pyrido[1,2-a]pyrimidin-9-yl)ethyl)amino)benzoic acid;
(R)-2-((1-(3,7-dimethyl-4-oxo-2-(4-(thiazol-2-yl) piperazin-1-yl)-4H-pyrido[1,2-a]pyrimidin-9-yl)ethyl)amino)benzoic acid;
(R)-2-((1-(3,7-dimethyl-4-oxo-2-(4-(pyridin-2-yl) piperazin-1-yl)-4H-pyrido[1,2-a]pyrimidin-9-yl)ethyl)amino)benzoic acid;
(R)-2-((1-(2-(4-(4,6-dicyanonaphthalen-1-yl) piperazin-1-yl)-3,7-dimethyl-4-oxo-4H-pyrido[1,2-a]pyrimidin-9-yl)ethyl)amino)benzoic acid;
(R)-2-((1-(2-(4-(4-fluorophenyl) piperazin-1-yl)-3,7-dimethyl-4-oxo-4H-pyrido[1,2-a]pyrimidin-9-yl)ethyl)amino)benzoic acid;
(R)-2-((1-(3-cyano-2-((3,3-difluorocyclobutyl)amino)-7-methyl-4-oxo-4H-pyrido[1,2-a]pyrimidin-9-yl)ethyl)amino)benzoic acid;
(R)-2-((1-(3,7-dimethyl-4-oxo-2-(4-(pyridin-3-yl) piperazin-1-yl)-4H-pyrido[1,2-a]pyrimidin-9-yl)ethyl)amino)benzoic acid;
(R)-2-((1-(3-cyano-7-fluoro-2-(isoindolin-2-yl)-4-oxo-4H-pyrido[1,2-a]pyrimidin-9-yl)ethyl)amino)benzoic acid;
(R)-2-((1-(3-cyano-2-(4-(4-fluorophenyl) piperazin-1-yl)-7-methyl-4-oxo-4H-pyrido[1,2-a]pyrimidin-9-yl)ethyl)amino)benzoic acid;
2-(((R)-1-(3-cyano-2-((S)-4-(4-cyanophenyl)-3-methylpiperazin-1-yl)-7-methyl-4-oxo-4H-pyrido[1,2-a]pyrimidin-9-yl)ethyl)amino)benzoic acid;
2-(((R)-1-(2-((R)-4-(4-cyanophenyl)-2-methylpiperazin-1-yl)-3,7-dimethyl-4-oxo-4H-pyrido[1,2-a]pyrimidin-9-yl)ethyl)amino)benzoic acid;
(R)-2-((1-(3-cyano-2-(isoindolin-2-yl)-7-methyl-4-oxo-4H-pyrido[1,2-a]pyrimidin-9-yl)ethyl)amino)benzoic acid;
2-(((1R)-1-(3-cyano-2-(3,3-difluoro-4-methylpyrrolidin-1-yl)-7-methyl-4-oxo-4H-pyrido[1,2-a]pyrimidin-9-yl)ethyl)amino)benzoic acid;
(R)-2-((1-(2-(4-(2-cyanophenyl) piperazin-1-yl)-3,7-dimethyl-4-oxo-4H-pyrido[1,2-a]pyrimidin-9-yl)ethyl)amino)benzoic acid;
(R)-2-((1-(3-cyano-2-(4,4-difluoropiperidin-1-yl)-7-ethyl-4-oxo-4H-pyrido[1,2-a]pyrimidin-9-yl)ethyl)amino)benzoic acid;
2-(((R)-1-(3-cyano-2-((S)-3,3-difluoro-4-methoxypiperidin-1-yl)-7-methyl-4-oxo-4H-pyrido[1,2-a]pyrimidin-9-yl)ethyl)amino)benzoic acid;
2-(((R)-1-(3-cyano-2-((R)-3,3-difluoro-4-methoxypiperidin-1-yl)-7-methyl-4-oxo-4H-pyrido[1,2-a]pyrimidin-9-yl)ethyl)amino)benzoic acid;
(R)-2-((1-(3,7-dimethyl-2-(4-(3-methylisoxazol-5-yl) piperazin-1-yl)-4-oxo-4H-pyrido[1,2-a]pyrimidin-9-yl)ethyl)amino)benzoic acid;
(R)-2-((1-(3-cyano-2-((3,3-difluorocyclobutyl)(methyl)amino)-7-methyl-4-oxo-4H-pyrido[1,2-a]pyrimidin-9-yl)ethyl)amino)benzoic acid;
(R)-6-chloro-3-((1-(3-cyano-2-(5-fluoroisoindolin-2-yl)-7-methyl-4-oxo-4H-pyrido[1,2-a]pyrimidin-9-yl)ethyl)amino) picolinic acid;
(R)-2-((1-(3,7-dimethyl-4-oxo-2-(3,3,4,4-tetrafluoropyrrolidin-1-yl)-4H-pyrido[1,2-a]pyrimidin-9-yl)ethyl)amino)benzoic acid;
2-(((R)-1-(3-cyano-2-((R)-4,4-difluoro-2-methylpyrrolidin-1-yl)-7-methyl-4-oxo-4H-pyrido[1,2-a]pyrimidin-9-yl)ethyl)amino)benzoic acid;
2-(((R)-1-(3-cyano-2-((S)-4,4-difluoro-2-methylpyrrolidin-1-yl)-7-methyl-4-oxo-4H-pyrido[1,2-a]pyrimidin-9-yl)ethyl)amino)benzoic acid;
2-(((R)-1-(3-cyano-2-((R)-2-(fluoromethyl)azetidin-1-yl)-7-methyl-4-oxo-4H-pyrido[1,2-a]pyrimidin-9-yl)ethyl)amino)benzoic acid;
2-(((R)-1-(3-cyano-2-((S)-2-(fluoromethyl)azetidin-1-yl)-7-methyl-4-oxo-4H-pyrido[1,2-a]pyrimidin-9-yl)ethyl)amino)benzoic acid;
(R)-2-((1-(2-(4-(4-cyano-2-fluorophenyl) piperazin-1-yl)-3,7-dimethyl-4-oxo-4H-pyrido[1,2-a]pyrimidin-9-yl)ethyl)amino)benzoic acid;
(R)-2-((1-(2-(4-methoxy-4-methylpiperidin-1-yl)-3,7-dimethyl-4-oxo-4H-pyrido[1,2-a]pyrimidin-9-yl)ethyl)amino)benzoic acid;
2-(((1R)-1-(3-cyano-7-methyl-2-(2-methyl-4-phenylpiperazin-1-yl)-4-oxo-4H-pyrido[1,2-a]pyrimidin-9-yl)ethyl)amino)benzoic acid;
(R)-2-((1-(3,7-dimethyl-2-(4-(1-methyl-1H-pyrazol-3-yl) piperidin-1-yl)-4-oxo-4H-pyrido[1,2-a]pyrimidin-9-yl)ethyl)amino)benzoic acid;
2-(((R)-1-(3-cyano-2-((1S,4S)-5,5-difluoro-2-azabicyclo[2.2.1]heptan-2-yl)-7-methyl-4-oxo-4H-pyrido[1,2-a]pyrimidin-9-yl)ethyl)amino) benzoic acid;
2-(((R)-1-(3-cyano-2-((1R,4R)-5,5-difluoro-2-azabicyclo[2.2.1]heptan-2-yl)-7-methyl-4-oxo-4H-pyrido[1,2-a]pyrimidin-9-yl)ethyl)amino) benzoic acid;
2-(((1R)-1-(3-cyano-2-(((2,2-difluoro-1-methylcyclopropyl)methyl)amino)-7-methyl-4-oxo-4H-pyrido[1,2-a]pyrimidin-9-yl)ethyl)amino)benzoic acid;
(R)-4-((1-(3-cyano-2-(4,4-difluoropiperidin-1-yl)-7-methyl-4-oxo-4H-pyrido[1,2-a]pyrimidin-9-yl)ethyl)amino) thiophene-3-carboxylic acid;
(R)-2-((1-(3-cyano-2-(4,4-difluoropiperidin-1-yl)-7-methyl-4-oxo-4H-pyrido[1,2-a]pyrimidin-9-yl)ethyl)amino)-6-fluorobenzoic acid;
(R)-3-((1-(3-cyano-2-(4,4-difluoropiperidin-1-yl)-7-methyl-4-oxo-4H-pyrido[1,2-a]pyrimidin-9-yl)ethyl)amino) pyrazine-2-carboxylic acid;
(R)-5-chloro-2-((1-(3-cyano-2-(4,4-difluoropiperidin-1-yl)-7-methyl-4-oxo-4H-pyrido[1,2-a]pyrimidin-9-yl)ethyl)amino)benzoic acid;
(R)-2-((1-(3-cyano-2-(4,4-difluorocyclohexyl)-7-methyl-4-oxo-4H-pyrido[1,2-a]pyrimidin-9-yl)ethyl)amino)benzoic acid;
(R)-2-((1-(3-cyano-2-(6,6-difluoro-2-azaspiro[3.3]heptan-2-yl)-7-methyl-4-oxo-4H-pyrido[1,2-a]pyrimidin-9-yl)ethyl)amino)benzoic acid;
(R)-6-chloro-3-((1-(3-cyano-2-(4,4-difluoropiperidin-1-yl)-7-methyl-4-oxo-4H-pyrido[1,2-a]pyrimidin-9-yl)ethyl)amino) picolinic acid;
(R)-2-((1-(3-cyano-2-(1,1-dioxidothiomorpholino)-7-methyl-4-oxo-4H-pyrido[1,2-a]pyrimidin-9-yl)ethyl)amino)benzoic acid;
2-(((1R)-1-(3-cyano-2-(6,6-difluoro-3-azabicyclo[3.1.1]heptan-3-yl)-7-methyl-4-oxo-4H-pyrido[1,2-a]pyrimidin-9-yl)ethyl)amino)benzoic acid;
(R)-2-((1-(3-carbamoyl-7-methyl-4-oxo-2-(piperidin-1-yl)-4H-pyrido[1,2-a]pyrimidin-9-yl)ethyl)amino)benzoic acid;
(R)-2-((1-(7-methyl-4-oxo-2-(piperidin-1-yl)-3-(trifluoromethyl)-4H-pyrido[1,2-a]pyrimidin-9-yl)ethyl)amino)benzoic acid;
(R)-2-((1-(3-cyano-2-(4,4-dimethylpiperidin-1-yl)-7-methyl-4-oxo-4H-pyrido[1,2-a]pyrimidin-9-yl)ethyl)amino)benzoic acid;
(R)-2-((1-(3-cyano-7-methyl-4-oxo-2-(pyrrolidin-1-yl)-4H-pyrido[1,2-a]pyrimidin-9-yl)ethyl)amino)benzoic acid;
(R)-2-((1-(3,7-dimethyl-4-oxo-2-(piperidin-1-yl)-4H-pyrido[1,2-a]pyrimidin-9-yl)ethyl)amino)benzoic acid;
(R)-2-((1-(3-cyano-2-(3,3-difluoropyrrolidin-1-yl)-7-methyl-4-oxo-4H-pyrido[1,2-a]pyrimidin-9-yl)ethyl)amino)benzoic acid;
(R)-2-((1-(2-(2-azabicyclo[2.1.1]hexan-2-yl)-3-cyano-7-methyl-4-oxo-4H-pyrido[1,2-a]pyrimidin-9-yl)ethyl)amino)benzoic acid;
(R)-2-((1-(2-(4-(4-cyano-2-methylphenyl) piperazin-1-yl)-3,7-dimethyl-4-oxo-4H-pyrido[1,2-a]pyrimidin-9-yl)ethyl)amino)benzoic acid;
(R)-2-((1-(3-cyano-2-(4-methoxy-4-methylpiperidin-1-yl)-7-methyl-4-oxo-4H-pyrido[1,2-a]pyrimidin-9-yl)ethyl)amino)benzoic acid;
(R)-2-((1-(3-fluoro-7-methyl-4-oxo-2-(piperidin-1-yl)-4H-pyrido[1,2-a]pyrimidin-9-yl)ethyl)amino)benzoic acid;
(R)-2-((1-(3-cyano-2-((cyclopropylmethyl)(methyl)amino)-7-methyl-4-oxo-4H-pyrido[1,2-a]pyrimidin-9-yl)ethyl)amino)benzoic acid;
(R)-2-((1-(3-cyano-7-methyl-4-oxo-2-(1,1,2,2-tetrafluoro-6-azaspiro[2.5]octan-6-yl)-4H-pyrido[1,2-a]pyrimidin-9-yl)ethyl)amino) benzoic acid;
(R)-2-((1-(2-(4-(4-chlorobenzoyl) piperazin-1-yl)-3,7-dimethyl-4-oxo-4H-pyrido[1,2-a]pyrimidin-9-yl)ethyl)amino)benzoic acid;
(R)-2-((1-(3-cyano-7-cyclopropyl-2-(4,4-difluoropiperidin-1-yl)-4-oxo-4H-pyrido[1,2-a]pyrimidin-9-yl)ethyl)amino)benzoic acid;
(R)-2-((1-(2-(4-(cyclopentanecarbonyl) piperazin-1-yl)-3,7-dimethyl-4-oxo-4H-pyrido[1,2-a]pyrimidin-9-yl)ethyl)amino)benzoic acid;
(R)-6-chloro-3-((1-(2-((cyclopropylmethyl)amino)-3,7-dimethyl-4-oxo-4H-pyrido[1,2-a]pyrimidin-9-yl)ethyl)amino) picolinic acid;
(R)-2-((1-(3-cyano-2-(4-(2,4-difluorophenyl) piperazin-1-yl)-7-methyl-4-oxo-4H-pyrido[1,2-a]pyrimidin-9-yl)ethyl)amino)benzoic acid;
(R)-2-((1-(3-cyano-2-(5,6-difluoroisoindolin-2-yl)-7-methyl-4-oxo-4H-pyrido[1,2-a]pyrimidin-9-yl)ethyl)amino)benzoic acid;
2-(((1R)-1-(3-cyano-2-(6,6-difluoro-2-azabicyclo[2.2.1]heptan-2-yl)-7-methyl-4-oxo-4H-pyrido[1,2-a]pyrimidin-9-yl)ethyl)amino)benzoic acid;
2-(((1R)-1-(2-(8-azabicyclo[3.2.1]octan-8-yl)-3-cyano-7-methyl-4-oxo-4H-pyrido[1,2-a]pyrimidin-9-yl)ethyl)amino)benzoic acid;
(R)-2-((1-(3-cyano-2-(cyclobutylamino)-7-methyl-4-oxo-4H-pyrido[1,2-a]pyrimidin-9-yl)ethyl)amino)benzoic acid;
(R)-2-((1-(2-(4-(2,2-difluoroethyl) piperazin-1-yl)-3,7-dimethyl-4-oxo-4H-pyrido[1,2-a]pyrimidin-9-yl)ethyl)amino)benzoic acid;
(R)-2-((1-(3-cyano-2-(5-fluoroisoindolin-2-yl)-7-methyl-4-oxo-4H-pyrido[1,2-a]pyrimidin-9-yl)ethyl)amino)benzoic acid;
(R)-2-((1-(2-(4-(2,4-difluorophenyl) piperazin-1-yl)-3,7-dimethyl-4-oxo-4H-pyrido[1,2-a]pyrimidin-9-yl)ethyl)amino)benzoic acid;
(R)-2-((1-(3-cyano-2-(4-(4-cyano-2-fluorophenyl) piperazin-1-yl)-7-methyl-4-oxo-4H-pyrido[1,2-a]pyrimidin-9-yl)ethyl)amino)benzoic acid;
2-(((R)-1-(3-cyano-7-methyl-2-((S)-2-methylazetidin-1-yl)-4-oxo-4H-pyrido[1,2-a]pyrimidin-9-yl)ethyl)amino)benzoic acid;
2-(((R)-1-(3-cyano-7-methyl-2-((R)-2-methylazetidin-1-yl)-4-oxo-4H-pyrido[1,2-a]pyrimidin-9-yl)ethyl)amino)benzoic acid;
(R)-2-((1-(2-(2-azabicyclo[2.2.2]octan-2-yl)-3-cyano-7-methyl-4-oxo-4H-pyrido[1,2-a]pyrimidin-9-yl)ethyl)amino)benzoic acid;
(R)-2-((1-(3-cyano-7-methyl-4-oxo-2-(4-phenylpiperazin-1-yl)-4H-pyrido[1,2-a]pyrimidin-9-yl)ethyl)amino)benzoic acid;
(R)-2-((1-(3-cyano-7-methyl-4-oxo-2-(piperidin-1-yl)-4H-pyrido[1,2-a]pyrimidin-9-yl)ethyl)amino)benzoic acid;
2-(((1R)-1-(3-cyano-2-(6,6-difluoro-3-azabicyclo[3.1.0]hexan-3-yl)-7-methyl-4-oxo-4H-pyrido[1,2-a]pyrimidin-9-yl)ethyl)amino)benzoic acid;
(R)-2-((1-(3-cyano-2-(4-(4-cyano-2-methylphenyl) piperazin-1-yl)-7-methyl-4-oxo-4H-pyrido[1,2-a]pyrimidin-9-yl)ethyl)amino)benzoic acid;
2-(((1R)-1-(2-(6,6-difluoro-3-azabicyclo[3.1.1]heptan-3-yl)-3,7-dimethyl-4-oxo-4H-pyrido[1,2-a]pyrimidin-9-yl)ethyl)amino)benzoic acid;
(R)-2-((1-(3-cyano-2-(1-(4-cyanophenyl) piperidin-4-yl)-7-methyl-4-oxo-4H-pyrido[1,2-a]pyrimidin-9-yl)ethyl)amino)benzoic acid;
(R)-2-((1-(3,7-dimethyl-4-oxo-2-(4-(3-(trifluoromethyl)-1H-pyrazol-1-yl) piperidin-1-yl)-4H-pyrido[1,2-a]pyrimidin-9-yl)ethyl)amino)benzoic acid;
(R)-3-((1-(2-(4,4-difluoropiperidin-1-yl)-3,7-dimethyl-4-oxo-4H-pyrido[1,2-a]pyrimidin-9-yl)ethyl)amino)-6-fluoropicolinic acid;
2-(((R)-1-(2-((S)-3,3-difluoro-4-methoxypiperidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-4-methoxypiperidin-1-yl)-3,7-dimethyl-4-oxo-4H-pyrido[1,2-a]pyrimidin-9-yl)ethyl)amino)benzoic acid;
(R)-2-((1-(2-(4,4-difluoro-6-azaspiro[2.5]octan-6-yl)-3, R 7-dimethyl-4-oxo-4H-pyrido[1,2-a]pyrimidin-9-yl)ethyl)amino)benzoic acid;
(R)-2-((1-(3-cyano-2-(4,4-difluoro-6-azaspiro[2.5]octan-6-yl)-7-methyl-4-oxo-4H-pyrido[1,2-a]pyrimidin-9-yl)ethyl)amino)benzoic acid;
2-(((1R)-1-(3-cyano-2-(4-(4-cyanophenyl)-2-methylpiperazin-1-yl)-7-methyl-4-oxo-4H-pyrido[1,2-a]pyrimidin-9-yl)ethyl)amino)benzoic acid;
2-(((1R)-1-(2-(4-(4-cyanophenyl)-3-methylpiperazin-1-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(2-oxa-7-azaspiro[3.5]nonan-7-yl)-4H-pyrido[1,2-a]pyrimidin-9-yl)ethyl)amino)benzoic acid;
(R)-2-((1-(3-cyano-2-(4-(5-cyanopyrimidin-2-yl) piperazin-1-yl)-7-methyl-4-oxo-4H-pyrido[1,2-a]pyrimidin-9-yl)ethyl)amino)benzoic acid;
2-(((R)-1-(2-((S)-3,3-difluoro-2-methylazetidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-2-methylazetidin-1-yl)-3,7-dimethyl-4-oxo-4H-pyrido[1,2-a]pyrimidin-9-yl)ethyl)amino)benzoic acid;
(R)-2-((1-(3-chloro-2-(4,4-difluoropiperidin-1-yl)-7-methyl-4-oxo-4H-pyrido[1,2-a]pyrimidin-9-yl)ethyl)amino)benzoic acid;
(R)-3-((1-(3-cyano-2-(cyclobutylamino)-7-methyl-4-oxo-4H-pyrido[1,2-a]pyrimidin-9-yl)ethyl)amino)-6-methylpicolinic acid;
2-(((1R)-1-(2-(3,3-difluoro-4-methoxypiperidin-1-yl)-3,7-dimethyl-4-oxo-4H-pyrido[1,2-a]pyrimidin-9-yl)ethyl)amino)benzoic acid;
(R)-2-((1-(2-(4-benzoylpiperazin-1-yl)-3,7-dimethyl-4-oxo-4H-pyrido[1,2-a]pyrimidin-9-yl)ethyl)amino)benzoic acid;
2-(((R)-1-(2-((1R,4R)-5,5-difluoro-2-azabicyclo[2.2.1]heptan-2-yl)-3,7-dimethyl-4-oxo-4H-pyrido[1,2-a]pyrimidin-9-yl)ethyl)amino)benzoic acid;
2-(((R)-1-(2-((1S,4S)-5,5-difluoro-2-azabicyclo[2.2.1]heptan-2-yl)-3,7-dimethyl-4-oxo-4H-pyrido[1,2-a]pyrimidin-9-yl)ethyl)amino)benzoic acid;
2-(((R)-1-(3-cyano-2-((R)-3,3-difluoro-4-methoxypyrrolidin-1-yl)-7-methyl-4-oxo-4H-pyrido[1,2-a]pyrimidin-9-yl)ethyl)amino)benzoic acid;
2-(((R)-1-(3-cyano-2-((S)-3,3-difluoro-4-methoxypyrrolidin-1-yl)-7-methyl-4-oxo-4H-pyrido[1,2-a]pyrimidin-9-yl)ethyl)amino)benzoic acid;
(R)-2-((1-(2-(1-(4-cyanophenyl) piperidin-4-yl)-3,7-dimethyl-4-oxo-4H-pyrido[1,2-a]pyrimidin-9-yl)ethyl)amino)benzoic acid;
(R)-3-((1-(3-cyano-2-(4,4-difluoropiperidin-1-yl)-7-methyl-4-oxo-4H-pyrido[1,2-a]pyrimidin-9-yl)ethyl)amino) picolinic acid;
2-(((R)-1-(3-cyano-2-((R)-4,4-difluoro-3-methylpiperidin-1-yl)-7-methyl-4-oxo-4H-pyrido[1,2-a]pyrimidin-9-yl)ethyl)amino)benzoic acid;
2-(((R)-1-(3-cyano-2-((S)-4,4-difluoro-3-methylpiperidin-1-yl)-7-methyl-4-oxo-4H-pyrido[1,2-a]pyrimidin-9-yl)ethyl)amino)benzoic acid;
(R)-2-((1-(3-cyano-2-(4-methoxypiperidin-1-yl)-7-methyl-4-oxo-4H-pyrido[1,2-a]pyrimidin-9-yl)ethyl)amino)benzoic acid;
(R)-2-((1-(2-(3-azabicyclo[3.1.1]heptan-3-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(4-(2,2,2-trifluoroethyl) piperazin-1-yl)-4H-pyrido[1,2-a]pyrimidin-9-yl)ethyl)amino)benzoic acid;
2-(((R)-1-(2-((R)-4,4-difluoro-3-methylpiperidin-1-yl)-3,7-dimethyl-4-oxo-4H-pyrido[1,2-a]pyrimidin-9-yl)ethyl)amino)benzoic acid;
2-(((R)-1-(2-((S)-4,4-difluoro-3-methylpiperidin-1-yl)-3,7-dimethyl-4-oxo-4H-pyrido[1,2-a]pyrimidin-9-yl)ethyl)amino)benzoic acid;
(R)-2-((1-(2-(5-(difluoromethyl) isoindolin-2-yl)-3,7-dimethyl-4-oxo-4H-pyrido[1,2-a]pyrimidin-9-yl)ethyl)amino)benzoic acid;
(R)-2-((1-(3,7-dimethyl-2-(4-(4-(N-methylsulfamoyl)phenyl) piperazin-1-yl)-4-oxo-4H-pyrido[1,2-a]pyrimidin-9-yl)ethyl)amino)benzoic acid;
(R)-2-((1-(3,7-dimethyl-4-oxo-2-(2-oxa-8-azaspiro[4.5]decan-8-yl)-4H-pyrido[1,2-a]pyrimidin-9-yl)ethyl)amino)benzoic acid;
(R)-2-((1-(2-(4-(2-cyanopyridin-3-yl) piperazin-1-yl)-3,7-dimethyl-4-oxo-4H-pyrido[1,2-a]pyrimidin-9-yl)ethyl)amino)benzoic acid;
2-(((1R)-1-(3,7-dimethyl-2-(2-methyl-4-(pyridin-3-ylmethyl) piperazin-1-yl)-4-oxo-4H-pyrido[1,2-a]pyrimidin-9-yl)ethyl)amino)benzoic acid;
2-(((1R)-1-(3,7-dimethyl-2-(octahydro-2H-4,7-ethanoisoindol-2-yl)-4-oxo-4H-pyrido[1,2-a]pyrimidin-9-yl)ethyl)amino)benzoic acid;
(R)-2-((1-(2-(5-fluoroisoindolin-2-yl)-3,7-dimethyl-4-oxo-4H-pyrido[1,2-a]pyrimidin-9-yl)ethyl)amino)benzoic acid;
2-(((1R)-1-(2-(3-hydroxy-3-isopropylpyrrolidin-1-yl)-3,7-dimethyl-4-oxo-4H-pyrido[1,2-a]pyrimidin-9-yl)ethyl)amino)benzoic acid;
2-(((1R)-1-(3,7-dimethyl-2-(5-methyl-2-azabicyclo[2.2.1]heptan-2-yl)-4-oxo-4H-pyrido[1,2-a]pyrimidin-9-yl)ethyl)amino)benzoic acid;
(R)-2-((1-(2-(5-(hydroxymethyl) isoindolin-2-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(2-oxa-7-azaspiro[4.4]nonan-7-yl)-4H-pyrido[1,2-a]pyrimidin-9-yl)ethyl)amino)benzoic acid;
(R)-2-((1-(2-(5,6-dimethoxyisoindolin-2-yl)-3,7-dimethyl-4-oxo-4H-pyrido[1,2-a]pyrimidin-9-yl)ethyl)amino)benzoic acid;
(R)-2-((1-(2-(5,6-difluoroisoindolin-2-yl)-3,7-dimethyl-4-oxo-4H-pyrido[1,2-a]pyrimidin-9-yl)ethyl)amino)benzoic acid;
2-(((1R)-1-(2-(6,6-difluoro-2-azabicyclo[2.2.1]heptan-2-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(3-(pyridin-2-yl) pyrrolidin-1-yl)-4H-pyrido[1,2-a]pyrimidin-9-yl)ethyl)amino)benzoic acid;
2-(((1R)-1-(2-(2-hydroxy-8-azaspiro[4.5]decan-8-yl)-3,7-dimethyl-4-oxo-4H-pyrido[1,2-a]pyrimidin-9-yl)ethyl)amino)benzoic acid;
2-(((1R)-1-(2-(1-(hydroxymethyl)-6-azabicyclo[3.2.1]octan-6-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(1-(trifluoromethyl)-3-azabicyclo[3.1.0]hexan-3-yl)-4H-pyrido[1,2-a]pyrimidin-9-yl)ethyl)amino)benzoic acid;
2-(((1R)-1-(2-(8-hydroxy-3-azabicyclo[3.2.1]octan-3-yl)-3,7-dimethyl-4-oxo-4H-pyrido[1,2-a]pyrimidin-9-yl)ethyl)amino)benzoic acid;
2-(((1R)-1-(2-(6-hydroxy-2-azabicyclo[2.2.2]octan-2-yl)-3,7-dimethyl-4-oxo-4H-pyrido[1,2-a]pyrimidin-9-yl)ethyl)amino)benzoic acid;
2-(((R)-1-(2-((3aS,6aS)-1H,3H,4H-3a,6a-(methanooxymethano) furo [3,4-c]pyrrol-5(6H)-yl)-3,7-dimethyl-4-oxo-4H-pyrido[1,2-a]pyrimidin-9-yl)ethyl)amino)benzoic acid; or
a pharmaceutically acceptable salt thereof.

20. The compound of claim 1, which is:
(R)-2-((1-(2-(4-(2-chlorophenyl) piperazin-1-yl)-3,7-dimethyl-4-oxo-4H-pyrido[1,2-a]pyrimidin-9-yl)ethyl)amino)benzoic acid;
2-(((1R)-1-(2-(4,4-difluoro-3-methylpiperidin-1-yl)-3,7-dimethyl-4-oxo-4H-pyrido[1,2-a]pyrimidin-9-yl)ethyl)amino)benzoic acid;
2-(((1R)-1-(2-(4,4-difluoro-3-(hydroxymethyl) piperidin-1-yl)-3,7-dimethyl-4-oxo-4H-pyrido[1,2-a]pyrimidin-9-yl)ethyl)amino)benzoic acid;
2-(((1R)-1-(2-(8,8-difluoro-3-azabicyclo[3.2.1]octan-3-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(3,4,4-trifluoropiperidin-1-yl)-4H-pyrido[1,2-a]pyrimidin-9-yl)ethyl)amino)benzoic acid;
2-(((1R)-1-(2-(9,9-difluoro-3-oxa-7-azabicyclo[3.3.1]nonan-7-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(7,7,9,9-tetrafluoro-3-azabicyclo[3.3.1]nonan-3-yl)-4H-pyrido[1,2-a]pyrimidin-9-yl)ethyl)amino)benzoic acid;
2-(((1R)-1-(2-(9,9-difluoro-3-azabicyclo[3.3.1]nonan-3-yl)-3,7-dimethyl-4-oxo-4H-pyrido[1,2-a]pyrimidin-9-yl)ethyl)amino)benzoic acid;
2-(((1R)-1-(2-(9,9-difluoro-1-(hydroxymethyl)-3-azabicyclo[3.3.1]nonan-3-yl)-3,7-dimethyl-4-oxo-4H-pyrido[1,2-a]pyrimidin-9-yl)ethyl)amino) benzoic acid;
2-(((R)-1-(2-((3R,5R)-4,4-difluoro-3,5-dimethylpiperidin-1-yl)-3,7-dimethyl-4-oxo-4H-pyrido[1,2-a]pyrimidin-9-yl)ethyl)amino)benzoic acid;
And
2-(((R)-1-(2-((3S,5S)-4,4-difluoro-3,5-dimethylpiperidin-1-yl)-3,7-dimethyl-4-oxo-4H-pyrido[1,2-a]pyrimidin-9-yl)ethyl)amino)benzoic acid;
2-(((1R)-1-(2-(5,5-difluoro-2-azabicyclo[2.2.2]octan-2-yl)-3,7-dimethyl-4-oxo-4H-pyrido[1,2-a]pyrimidin-9-yl)ethyl)amino)benzoic acid;
2-(((R)-1-(2-((3R,5S)-4,4-difluoro-3,5-dimethylpiperidin-1-yl)-3,7-dimethyl-4-oxo-4H-pyrido[1,2-a]pyrimidin-9-yl)ethyl)amino)benzoic acid;
And
2-(((R)-1-(2-((3S,5R)-4,4-difluoro-3,5-dimethylpiperidin-1-yl)-3,7-dimethyl-4-oxo-4H-pyrido[1,2-a]pyrimidin-9-yl)ethyl)amino)benzoic acid;
2-(((1R)-1-(2-(7-(tert-butyl)-6,7-dihydropyrazolo[1,5-a]pyrimidin-4(5H)-yl)-3,7-dimethyl-4-oxo-4H-pyrido[1,2-a]pyrimidin-9-yl)ethyl)amino) benzoic acid;
2-(((1R)-1-(3,7-dimethyl-4-oxo-2-(3-phenylpyrrolidin-1-yl)-4H-pyrido[1,2-a]pyrimidin-9-yl)ethyl)amino)benzoic acid;
(R)-2-((1-(3,7-dimethyl-2-(2-methyl-4,6-dihydro-5H-pyrrolo[3,4-d]thiazol-5-yl)-4-oxo-4H-pyrido[1,2-a]pyrimidin-9-yl)ethyl)amino)benzoic acid;
(R)-2-((1-(3,7-dimethyl-4-oxo-2-(3-(pyridin-2-yl)azetidin-1-yl)-4H-pyrido[1,2-a]pyrimidin-9-yl)ethyl)amino)benzoic acid;
(R)-2-((1-(3,7-dimethyl-2-(5-methylisoindolin-2-yl)-4-oxo-4H-pyrido[1,2-a]pyrimidin-9-yl)ethyl)amino)benzoic acid;
(R)-2-((1-(3,7-dimethyl-4-oxo-2-(3-phenylazetidin-1-yl)-4H-pyrido[1,2-a]pyrimidin-9-yl)ethyl)amino)benzoic acid;
2-(((1R)-1-(2-(1-(hydroxymethyl) isoindolin-2-yl)-3,7-dimethyl-4-oxo-4H-pyrido[1,2-a]pyrimidin-9-yl)ethyl)amino)benzoic acid;
(R)-2-((1-(2-(4-cyclobutylpiperidin-1-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(3-(pyridin-3-yl)azetidin-1-yl)-4H-pyrido[1,2-a]pyrimidin-9-yl)ethyl)amino)benzoic acid;
2-(((1R)-1-(2-(6-hydroxyhexahydro-3,5-methanocyclopenta[b]pyrrol-1(2H)-yl)-3,7-dimethyl-4-oxo-4H-pyrido[1,2-a]pyrimidin-9-yl)ethyl)amino)benzoic acid;
(R)-2-((1-(2-(4,4-difluoro-[1,4′-bipiperidin]-1′-yl)-3,7-dimethyl-4-oxo-4H-pyrido[1,2-a]pyrimidin-9-yl)ethyl)amino)benzoic acid;
2-(((1R)-1-(2-(6,6-dimethyl-3-azabicyclo[3.2.0]heptan-3-yl)-3,7-dimethyl-4-oxo-4H-pyrido[1,2-a]pyrimidin-9-yl)ethyl)amino)benzoic acid;
(R)-2-((1-(2-(4-cyclopentylpiperidin-1-yl)-3,7-dimethyl-4-oxo-4H-pyrido[1,2-a]pyrimidin-9-yl)ethyl)amino)benzoic acid;
(R)-2-((1-(2-(2-azadispiro[3.1.36, 14]decan-2-yl)-3,7-dimethyl-4-oxo-4H-pyrido[1,2-a]pyrimidin-9-yl)ethyl)amino)benzoic acid;
2-(((1R)-1-(2-(decahydro-2H-cycloocta [c]pyrrol-2-yl)-3,7-dimethyl-4-oxo-4H-pyrido[1,2-a]pyrimidin-9-yl)ethyl)amino)benzoic acid;
2-(((1R)-1-(2-(8-hydroxy-6-azaspiro[3.4]octan-6-yl)-3,7-dimethyl-4-oxo-4H-pyrido[1,2-a]pyrimidin-9-yl)ethyl)amino)benzoic acid;
(R)-2-((1-(2-(2-azaadamantan-2-yl)-3,7-dimethyl-4-oxo-4H-pyrido[1,2-a]pyrimidin-9-yl)ethyl)amino)benzoic acid;
2-(((1R)-1-(2-(5-hydroxy-2-azabicyclo[2.2.2]octan-2-yl)-3,7-dimethyl-4-oxo-4H-pyrido[1,2-a]pyrimidin-9-yl)ethyl)amino)benzoic acid;
2-(((1R)-1-(2-(3-hydroxyoctahydro-1H-indol-1-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(5-phenyl-2-azabicyclo[2.2.1]heptan-2-yl)-4H-pyrido[1,2-a]pyrimidin-9-yl)ethyl)amino)benzoic acid;
2-(((1R)-1-(3,7-dimethyl-4-oxo-2-(8-azatricyclo[4.3.0.02.5]nonan-8-yl)-4H-pyrido[1,2-a]pyrimidin-9-yl)ethyl)amino)benzoic acid;
2-(((1R)-1-(2-(6-hydroxy-6-methyl-3-azabicyclo[3.1.1]heptan-3-yl)-3,7-dimethyl-4-oxo-4H-pyrido[1,2-a]pyrimidin-9-yl)ethyl)amino)benzoic acid;
2-(((1R)-1-(2-(8-hydroxyoctahydro-2H-4,7-methanoisoindol-2-yl)-3,7-dimethyl-4-oxo-4H-pyrido[1,2-a]pyrimidin-9-yl)ethyl)amino)benzoic acid;
(R)-2-((1-(2-(4-(bicyclo[1.1.1]pentan-1-yl-3-d) piperidin-1-yl)-3,7-dimethyl-4-oxo-4H-pyrido[1,2-a]pyrimidin-9-yl)ethyl)amino)benzoic acid;
(R)-2-((1-(2-(4-(2,6-dimethylphenyl) piperazin-1-yl)-3,7-dimethyl-4-oxo-4H-pyrido[1,2-a]pyrimidin-9-yl)ethyl)amino)benzoic acid;
(R)-2-((1-(2-(4-fluoroisoindolin-2-yl)-3,7-dimethyl-4-oxo-4H-pyrido[1,2-a]pyrimidin-9-yl)ethyl)amino)benzoic acid;
(R)-2-((1-(2-(4-(4-methoxyphenyl) piperidin-1-yl)-3,7-dimethyl-4-oxo-4H-pyrido[1,2-a]pyrimidin-9-yl)ethyl)amino)benzoic acid;
1-(2-(isoindolin-2-yl)-7-methyl-4-oxo-4H-pyrido[1,2-a]pyrimidin-9-yl)-1,2,3,4-tetrahydroquinoline-3-carboxylic acid;
2-(4-(2-(isoindolin-2-yl)-7-methyl-4-oxo-4H-pyrido[1,2-a]pyrimidin-9-yl)-7-oxo-1,4-diazepan-1-yl)acetic acid;
(R)-2-((1-(3-cyano-2-(4-(2,2-difluoroethyl) piperazin-1-yl)-7-methyl-4-oxo-4H-pyrido[1,2-a]pyrimidin-9-yl)ethyl)amino)benzoic acid;
(S)-3-((1-(3-cyano-2-(4,4-difluoropiperidin-1-yl)-7-methyl-4-oxo-4H-pyrido[1,2-a]pyrimidin-9-yl)ethyl)amino)-6-fluoropicolinic acid;
(R)-3-((1-(3-cyano-2-(4,4-difluoropiperidin-1-yl)-7-methyl-4-oxo-4H-pyrido[1,2-a]pyrimidin-9-yl)ethyl)amino)-6-fluoropicolinic acid;
(R)-3-((1-(2-(4,4-difluoropiperidin-1-yl)-3,7-dimethyl-4-oxo-4H-pyrido[1,2-a]pyrimidin-9-yl)ethyl)amino)-6-fluoropicolinic acid;
(R)-3-((1-(2-(4,4-difluoropiperidin-1-yl)-3,7-dimethyl-4-oxo-4H-pyrido[1,2-a]pyrimidin-9-yl)ethyl)amino)-6-methylpicolinic acid;
2-(((R)-1-(3-cyano-2-((3R,5R)-3,5-difluoropiperidin-1-yl)-7-methyl-4-oxo-4H-pyrido[1,2-a]pyrimidin-9-yl)ethyl)amino)benzoic acid;
2-(((R)-1-(3-cyano-2-((3R,5S)-3,5-difluoropiperidin-1-yl)-7-methyl-4-oxo-4H-pyrido[1,2-a]pyrimidin-9-yl)ethyl)amino)benzoic acid;
2-(((1R)-1-(3-cyano-7-methyl-4-oxo-2-((3,3,3-trifluoro-2-methylpropyl)amino)-4H-pyrido[1,2-a]pyrimidin-9-yl)ethyl)amino)benzoic acid;
(R)-6-chloro-3-((1-(7-chloro-3-cyano-2-(4,4-difluoropiperidin-1-yl)-4-oxo-4H-pyrido[1,2-a]pyrimidin-9-yl)ethyl)amino) picolinic acid;
2-(((R)-1-(3-cyano-7-methyl-4-oxo-2-(((R)-1-(pyridin-3-yl)ethyl)amino)-4H-pyrido[1,2-a]pyrimidin-9-yl)ethyl)amino)benzoic acid;
2-(((R)-1-(3-cyano-7-methyl-4-oxo-2-(((S)-1-(pyridin-3-yl)ethyl)amino)-4H-pyrido[1,2-a]pyrimidin-9-yl)ethyl)amino)benzoic acid;
(R)-2-((1-(2-(4-(1H-pyrazol-3-yl) piperidin-1-yl)-3-cyano-7-methyl-4-oxo-4H-pyrido[1,2-a]pyrimidin-9-yl)ethyl)amino)benzoic acid;
(R)-2-((1-(2-(2,2-difluoro-7-azaspiro[3.5]nonan-7-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(1-oxa-7-azaspiro[3.5]nonan-7-yl)-4H-pyrido[1,2-a]pyrimidin-9-yl)ethyl)amino)benzoic acid;
(R)-2-((1-(3,7-dimethyl-4-oxo-2-(1-oxa-7-azaspiro[3.5]nonan-7-yl)-4H-pyrido[1,2-a]pyrimidin-9-yl)ethyl)amino)benzoic acid;
2-(((R)-1-(2-((S)-4,4-difluoro-3-methoxypiperidin-1-yl)-3,7-dimethyl-4-oxo-4H-pyrido[1,2-a]pyrimidin-9-yl)ethyl)amino)benzoic acid;
2-(((R)-1-(2-((R)-4,4-difluoro-3-methoxypiperidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-4-methylpiperidin-1-yl)-3,7-dimethyl-4-oxo-4H-pyrido[1,2-a]pyrimidin-9-yl)ethyl)amino)benzoic acid;
2-(((R)-1-(2-((S)-3,3-difluoro-4-methylpiperidin-1-yl)-3,7-dimethyl-4-oxo-4H-pyrido[1,2-a]pyrimidin-9-yl)ethyl)amino)benzoic acid;
(R)-2-((1-(2-(7,7-difluoro-5-azaspiro[2.4]heptan-5-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(2-oxa-8-azaspiro[4.5]decan-8-yl)-4H-pyrido[1,2-a]pyrimidin-9-yl)ethyl)amino)benzoic acid;
(R)-2-((1-(3,7-dimethyl-4-oxo-2-(1-oxa-8-azaspiro[4.5]decan-8-yl)-4H-pyrido[1,2-a]pyrimidin-9-yl)ethyl)amino)benzoic acid;
2-(((R)-1-(3-cyano-2-((R)-4,4-difluoro-3-methylpiperidin-1-yl)-7-methyl-4-oxo-4H-pyrido[1,2-a]pyrimidin-9-yl)ethyl)amino)benzoic acid;
2-(((1R)-1-(2-(4,4-difluoro-3-methylpiperidin-1-yl)-3,7-dimethyl-4-oxo-4H-pyrido[1,2-a]pyrimidin-9-yl)ethyl)amino)benzoic acid;
2-(((R)-1-(3-cyano-2-((R)-3,3-difluoro-4-methoxypiperidin-1-yl)-7-methyl-4-oxo-4H-pyrido[1,2-a]pyrimidin-9-yl)ethyl)amino)benzoic acid;
2-(((1R)-1-(3-cyano-2-(3,3-difluoro-4-methoxypiperidin-1-yl)-7-methyl-4-oxo-4H-pyrido[1,2-a]pyrimidin-9-yl)ethyl)amino)benzoic acid;
(R)-2-((1-(3-cyano-2-(4-cyano-4-methylpiperidin-1-yl)-7-methyl-4-oxo-4H-pyrido[1,2-a]pyrimidin-9-yl)ethyl)amino)benzoic acid;
(R)-2-((1-(2-(4-cyano-4-methylpiperidin-1-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(4-(trifluoromethyl) piperidin-1-yl)-4H-pyrido[1,2-a]pyrimidin-9-yl)ethyl)amino)benzoic acid;
(R)-2-((1-(3,7-dimethyl-4-oxo-2-(4-(trifluoromethyl) piperidin-1-yl)-4H-pyrido[1,2-a]pyrimidin-9-yl)ethyl)amino)benzoic acid;
(R)-2-((1-(3-cyano-2-(4-cyanopiperidin-1-yl)-7-methyl-4-oxo-4H-pyrido[1,2-a]pyrimidin-9-yl)ethyl)amino)benzoic acid;
(R)-2-((1-(2-(4-cyanopiperidin-1-yl)-3,7-dimethyl-4-oxo-4H-pyrido[1,2-a]pyrimidin-9-yl)ethyl)amino)benzoic acid;
2-(((R)-1-(2-((R)-4-(2-cyano-5-methylphenyl)-2-methylpiperazin-1-yl)-3,7-dimethyl-4-oxo-4H-pyrido[1,2-a]pyrimidin-9-yl)ethyl)amino)benzoic acid;
2-(((R)-1-(2-((S)-4-(2-cyano-5-methylphenyl)-2-methylpiperazin-1-yl)-3,7-dimethyl-4-oxo-4H-pyrido[1,2-a]pyrimidin-9-yl)ethyl)amino)benzoic acid;
2-(((R)-1-(3-cyano-2-((R)-4-(2-cyano-4-(trifluoromethyl)phenyl)-2-methylpiperazin-1-yl)-7-methyl-4-oxo-4H-pyrido[1,2-a]pyrimidin-9-yl)ethyl)amino)benzoic acid;
2-(((R)-1-(3-cyano-2-((S)-4-(2-cyano-4-(trifluoromethyl)phenyl)-2-methylpiperazin-1-yl)-7-methyl-4-oxo-4H-pyrido[1,2-a]pyrimidin-9-yl)ethyl)amino)benzoic acid;
2-(((1R)-1-(3-cyano-2-(4-(2-cyano-4-(trifluoromethyl)phenyl)-2-methylpiperazin-1-yl)-7-methyl-4-oxo-4H-pyrido[1,2-a]pyrimidin-9-yl)ethyl)amino)benzoic acid;
2-(((R)-1-(2-((R)-4-(2-cyano-4-(trifluoromethyl)phenyl)-2-methylpiperazin-1-yl)-3,7-dimethyl-4-oxo-4H-pyrido[1,2-a]pyrimidin-9-yl)ethyl)amino)benzoic acid;
2-(((R)-1-(2-((S)-4-(2-cyano-4-(trifluoromethyl)phenyl)-2-methylpiperazin-1-yl)-3,7-dimethyl-4-oxo-4H-pyrido[1,2-a]pyrimidin-9-yl)ethyl)amino)benzoic acid;
2-(((1R)-1-(2-(4-(2-cyano-4-(trifluoromethyl)phenyl)-2-methylpiperazin-1-yl)-3,7-dimethyl-4-oxo-4H-pyrido[1,2-a]pyrimidin-9-yl)ethyl)amino) benzoic acid;
2-(((R)-1-(2-((S)-4-(2-cyanophenyl)-2-methylpiperazin-1-yl)-3,7-dimethyl-4-oxo-4H-pyrido[1,2-a]pyrimidin-9-yl)ethyl)amino)benzoic acid;
2-(((R)-1-(2-((R)-4-(2-cyanophenyl)-2-methylpiperazin-1-yl)-3,7-dimethyl-4-oxo-4H-pyrido[1,2-a]pyrimidin-9-yl)ethyl)amino)benzoic acid;
2-(((R)-1-(3-cyano-2-((R)-4-(3-cyanophenyl)-2-methylpiperazin-1-yl)-7-methyl-4-oxo-4H-pyrido[1,2-a]pyrimidin-9-yl)ethyl)amino)benzoic acid;
2-(((R)-1-(3-cyano-2-((S)-4-(3-cyanophenyl)-2-methylpiperazin-1-yl)-7-methyl-4-oxo-4H-pyrido[1,2-a]pyrimidin-9-yl)ethyl)amino)benzoic acid;
2-(((R)-1-(2-((S)-4-(3-cyanophenyl)-2-methylpiperazin-1-yl)-3,7-dimethyl-4-oxo-4H-pyrido[1,2-a]pyrimidin-9-yl)ethyl)amino)benzoic acid;
2-(((R)-1-(2-((R)-4-(3-cyanophenyl)-2-methylpiperazin-1-yl)-3,7-dimethyl-4-oxo-4H-pyrido[1,2-a]pyrimidin-9-yl)ethyl)amino)benzoic acid;
(R)-6-((1-(3-cyano-2-(4,4-difluoropiperidin-1-yl)-7-methyl-4-oxo-4H-pyrido[1,2-a]pyrimidin-9-yl)ethyl)amino)-2,3-difluorobenzoic acid;
(R)-3-chloro-6-((1-(3-cyano-2-(4,4-difluoropiperidin-1-yl)-7-methyl-4-oxo-4H-pyrido[1,2-a]pyrimidin-9-yl)ethyl)amino)-2-fluorobenzoic acid;
(R)-2-((1-(3-cyano-2-(4,4-difluoropiperidin-1-yl)-7-methyl-4-oxo-4H-pyrido[1,2-a]pyrimidin-9-yl)ethyl)amino)-5-fluorobenzoic acid;
(R)-5-chloro-2-((1-(2-(4,4-difluoropiperidin-1-yl)-3,7-dimethyl-4-oxo-4H-pyrido[1,2-a]pyrimidin-9-yl)ethyl)amino)benzoic acid;
2-(((R)-1-(2-((S)-4,4-difluoro-2-methylpiperidin-1-yl)-3,7-dimethyl-4-oxo-4H-pyrido[1,2-a]pyrimidin-9-yl)ethyl)amino)benzoic acid;
2-(((R)-1-(2-((R)-4,4-difluoro-2-methylpiperidin-1-yl)-3,7-dimethyl-4-oxo-4H-pyrido[1,2-a]pyrimidin-9-yl)ethyl)amino)benzoic acid;
2-(((R)-1-(2-((S)-2-(fluoromethyl)azetidin-1-yl)-3,7-dimethyl-4-oxo-4H-pyrido[1,2-a]pyrimidin-9-yl)ethyl)amino)benzoic acid;
2-(((R)-1-(2-((R)-2-(fluoromethyl)azetidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-2-methylazetidin-1-yl)-3,7-dimethyl-4-oxo-4H-pyrido[1,2-a]pyrimidin-9-yl)ethyl)amino)benzoic acid;
2-(((R)-1-(3,7-dimethyl-4-oxo-2-((S)-2-(trifluoromethyl)azetidin-1-yl)-4H-pyrido[1,2-a]pyrimidin-9-yl)ethyl)amino)benzoic acid;
2-(((R)-1-(3,7-dimethyl-4-oxo-2-((R)-2-(trifluoromethyl)azetidin-1-yl)-4H-pyrido[1,2-a]pyrimidin-9-yl)ethyl)amino)benzoic acid;
(R)-2-((1-(2-(cyclobutylamino)-3,7-dimethyl-4-oxo-4H-pyrido[1,2-a]pyrimidin-9-yl)ethyl)amino)benzoic acid;
2-(((R)-1-(3,7-dimethyl-2-((R)-2-methylazetidin-1-yl)-4-oxo-4H-pyrido[1,2-a]pyrimidin-9-yl)ethyl)amino)benzoic acid;
2-(((R)-1-(3,7-dimethyl-2-((S)-2-methylazetidin-1-yl)-4-oxo-4H-pyrido[1,2-a]pyrimidin-9-yl)ethyl)amino)benzoic acid;
(R)-2-((1-(3-cyano-2-(1,1-difluoro-5-azaspiro[2.3]hexan-5-yl)-7-methyl-4-oxo-4H-pyrido[1,2-a]pyrimidin-9-yl)ethyl)amino)benzoic acid;
(R)-2-((1-(2-(1,1-difluoro-5-azaspiro[2.3]hexan-5-yl)-3,7-dimethyl-4-oxo-4H-pyrido[1,2-a]pyrimidin-9-yl)ethyl)amino)benzoic acid;
(R)-2-((1-(3-cyano-2-(3-(difluoromethyl)azetidin-1-yl)-7-methyl-4-oxo-4H-pyrido[1,2-a]pyrimidin-9-yl)ethyl)amino)benzoic acid;
(R)-2-((1-(2-(3-(difluoromethyl)azetidin-1-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(3-(trifluoromethyl)azetidin-1-yl)-4H-pyrido[1,2-a]pyrimidin-9-yl)ethyl)amino)benzoic acid;
(R)-2-((1-(3,7-dimethyl-4-oxo-2-(3-(trifluoromethyl)azetidin-1-yl)-4H-pyrido[1,2-a]pyrimidin-9-yl)ethyl)amino)benzoic acid;
(R)-2-((1-(3-cyano-2-((2-cyanoallyl)amino)-7-methyl-4-oxo-4H-pyrido[1,2-a]pyrimidin-9-yl)ethyl)amino)benzoic acid;
(R)-2-((1-(3-cyano-2-(3-cyanoazetidin-1-yl)-7-methyl-4-oxo-4H-pyrido[1,2-a]pyrimidin-9-yl)ethyl)amino)benzoic acid;
(R)-2-((1-(2-((2-cyanoallyl)amino)-3,7-dimethyl-4-oxo-4H-pyrido[1,2-a]pyrimidin-9-yl)ethyl)amino)benzoic acid;
(R)-2-((1-(3-cyano-2-(3-methoxyazetidin-1-yl)-7-methyl-4-oxo-4H-pyrido[1,2-a]pyrimidin-9-yl)ethyl)amino)benzoic acid;
(R)-2-((1-(2-(3-methoxyazetidin-1-yl)-3,7-dimethyl-4-oxo-4H-pyrido[1,2-a]pyrimidin-9-yl)ethyl)amino)benzoic acid;
(R)-2-((1-(3-cyano-2-(3-fluoroazetidin-1-yl)-7-methyl-4-oxo-4H-pyrido[1,2-a]pyrimidin-9-yl)ethyl)amino)benzoic acid;
(R)-2-((1-(2-(3-fluoroazetidin-1-yl)-3,7-dimethyl-4-oxo-4H-pyrido[1,2-a]pyrimidin-9-yl)ethyl)amino)benzoic acid;
(R)-2-((1-(2-(3,3-difluoroazetidin-1-yl)-3,7-dimethyl-4-oxo-4H-pyrido[1,2-a]pyrimidin-9-yl)ethyl)amino)benzoic acid;
2-(((R)-1-(3-cyano-2-((S)-3,3-difluoro-4-methoxypyrrolidin-1-yl)-7-methyl-4-oxo-4H-pyrido[1,2-a]pyrimidin-9-yl)ethyl)amino)benzoic acid;
2-(((R)-1-(2-((R)-3,3-difluoro-4-methoxypyrrolidin-1-yl)-3,7-dimethyl-4-oxo-4H-pyrido[1,2-a]pyrimidin-9-yl)ethyl)amino)benzoic acid;
2-(((R)-1-(2-((S)-3,3-difluoro-4-methoxypyrrolidin-1-yl)-3,7-dimethyl-4-oxo-4H-pyrido[1,2-a]pyrimidin-9-yl)ethyl)amino)benzoic acid;
2-(((R)-1-(2-((S)-3,3-difluoro-4-methylpyrrolidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-4-methylpyrrolidin-1-yl)-3,7-dimethyl-4-oxo-4H-pyrido[1,2-a]pyrimidin-9-yl)ethyl)amino)benzoic acid;
2-(((1R)-1-(2-(3,3-difluoro-4-methylpyrrolidin-1-yl)-3,7-dimethyl-4-oxo-4H-pyrido[1,2-a]pyrimidin-9-yl)ethyl)amino)benzoic acid;
2-(((R)-1-(2-((S)-3,3-difluoro-2-methylpyrrolidin-1-yl)-3,7-dimethyl-4-oxo-4H-pyrido[1,2-a]pyrimidin-9-yl)ethyl)amino)benzoic acid;
2-(((R)-1-(2-((R)-3,3-difluoro-2-methylpyrrolidin-1-yl)-3,7-dimethyl-4-oxo-4H-pyrido[1,2-a]pyrimidin-9-yl)ethyl)amino)benzoic acid;
2-(((R)-1-(3-cyano-2-((S)-4,4-difluoro-2-methylpyrrolidin-1-yl)-7-methyl-4-oxo-4H-pyrido[1,2-a]pyrimidin-9-yl)ethyl)amino)benzoic acid;
2-(((R)-1-(2-((R)-4,4-difluoro-2-methylpyrrolidin-1-yl)-3,7-dimethyl-4-oxo-4H-pyrido[1,2-a]pyrimidin-9-yl)ethyl)amino)benzoic acid;
2-(((R)-1-(2-((S)-4,4-difluoro-2-methylpyrrolidin-1-yl)-3,7-dimethyl-4-oxo-4H-pyrido[1,2-a]pyrimidin-9-yl)ethyl)amino)benzoic acid;
(R)-2-((1-(2-(2-azabicyclo[2.1.1]hexan-2-yl)-3,7-dimethyl-4-oxo-4H-pyrido[1,2-a]pyrimidin-9-yl)ethyl)amino)benzoic acid;
6-chloro-3-(((R)-1-(3-cyano-2-((R)-4-(4-cyanophenyl)-2-methylpiperazin-1-yl)-7-methyl-4-oxo-4H-pyrido[1,2-a]pyrimidin-9-yl)ethyl)amino) picolinic acid;
6-chloro-3-(((R)-1-(3-cyano-2-((S)-4-(4-cyanophenyl)-2-methylpiperazin-1-yl)-7-methyl-4-oxo-4H-pyrido[1,2-a]pyrimidin-9-yl)ethyl)amino) picolinic acid;
6-chloro-3-(((R)-1-(3-cyano-2-((S)-4-(4-fluorophenyl)-2-methylpiperazin-1-yl)-7-methyl-4-oxo-4H-pyrido[1,2-a]pyrimidin-9-yl)ethyl)amino) picolinic acid;
6-chloro-3-(((R)-1-(3-cyano-2-((R)-4-(4-fluorophenyl)-2-methylpiperazin-1-yl)-7-methyl-4-oxo-4H-pyrido[1,2-a]pyrimidin-9-yl)ethyl)amino) picolinic acid;
(R)-6-chloro-3-((1-(3-cyano-2-(4-(4-cyano-2-methylphenyl) piperazin-1-yl)-7-methyl-4-oxo-4H-pyrido[1,2-a]pyrimidin-9-yl)ethyl)amino) picolinic acid;
(R)-6-chloro-3-((1-(2-(4-(4-cyano-2-methylphenyl) piperazin-1-yl)-3,7-dimethyl-4-oxo-4H-pyrido[1,2-a]pyrimidin-9-yl)ethyl)amino) picolinic acid;
2-(((1R)-1-(3-cyano-2-(6-(4-cyanophenyl)-3,6-diazabicyclo[3.1.1]heptan-3-yl)-7-methyl-4-oxo-4H-pyrido[1,2-a]pyrimidin-9-yl)ethyl)amino)benzoic acid;
2-(((1R)-1-(2-(6-(4-cyanophenyl)-3,6-diazabicyclo[3.1.1]heptan-3-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(3,3,4,4-tetrafluoropyrrolidin-1-yl)-4H-pyrido[1,2-a]pyrimidin-9-yl)ethyl)amino)benzoic acid;
(R)-2-((1-(3-cyano-2-(5,7-dihydro-6H-pyrrolo[3,4-b]pyrazin-6-yl)-7-methyl-4-oxo-4H-pyrido[1,2-a]pyrimidin-9-yl)ethyl)amino)benzoic acid;
(R)-2-((1-(2-(5,7-dihydro-6H-pyrrolo[3,4-b]pyrazin-6-yl)-3,7-dimethyl-4-oxo-4H-pyrido[1,2-a]pyrimidin-9-yl)ethyl)amino)benzoic acid;
(R)-6-chloro-3-((1-(2-(5,7-dihydro-6H-pyrrolo[3,4-b]pyrazin-6-yl)-3,7-dimethyl-4-oxo-4H-pyrido[1,2-a]pyrimidin-9-yl)ethyl)amino) picolinic acid;
(R)-2-((1-(2-(3,3-difluoropyrrolidin-1-yl)-3,7-dimethyl-4-oxo-4H-pyrido[1,2-a]pyrimidin-9-yl)ethyl)amino)benzoic acid;
(R)-2-((1-(2-(4,4-difluorocyclohexyl)-3,7-dimethyl-4-oxo-4H-pyrido[1,2-a]pyrimidin-9-yl)ethyl)amino)benzoic acid;
(R)-4-((1-(3-cyano-2-(4,4-difluoropiperidin-1-yl)-7-methyl-4-oxo-4H-pyrido[1,2-a]pyrimidin-9-yl)ethyl)amino) nicotinic acid;
6-chloro-3-(((1R)-1-(3-cyano-2-(6,6-difluoro-3-azabicyclo[3.1.0]hexan-3-yl)-7-methyl-4-oxo-4H-pyrido[1,2-a]pyrimidin-9-yl)ethyl)amino) picolinic acid;
(R)-6-chloro-3-((1-(3-cyano-2-(5,6-difluoroisoindolin-2-yl)-7-methyl-4-oxo-4H-pyrido[1,2-a]pyrimidin-9-yl)ethyl)amino) picolinic acid;
(R)-2-(4,4-difluoropiperidin-1-yl)-7-methyl-4-oxo-9-(1-(pyridazin-4-ylamino)ethyl)-4H-pyrido[1,2-a]pyrimidine-3-carbonitrile (R)-4-((1-(3-cyano-2-(4,4-difluoropiperidin-1-yl)-7-methyl-4-oxo-4H-pyrido[1,2-a]pyrimidin-9-yl)ethyl)amino)pyridazine-3-carboxylic acid;
(R)-6-chloro-3-((1-(3-cyano-2-(1,3-dihydro-2H-pyrrolo[3,4-c]pyridin-2-yl)-7-methyl-4-oxo-4H-pyrido[1,2-a]pyrimidin-9-yl)ethyl)amino) picolinic acid;
(R)-2-((1-(3,7-dimethyl-2-(1-methyl-1,2,3,6-tetrahydropyridin-4-yl)-4-oxo-4H-pyrido[1,2-a]pyrimidin-9-yl)ethyl)amino)benzoic acid;
(R)-2-((1-(3,7-dimethyl-2-(1-methylpiperidin-4-yl)-4-oxo-4H-pyrido[1,2-a]pyrimidin-9-yl)ethyl)amino)benzoic acid;
(R)-2-((1-(3-cyano-2-(4-cyclopropylpiperazin-1-yl)-7-methyl-4-oxo-4H-pyrido[1,2-a]pyrimidin-9-yl)ethyl)amino)benzoic acid;
(R)-2-((1-(2-(4-cyclopropylpiperazin-1-yl)-3,7-dimethyl-4-oxo-4H-pyrido[1,2-a]pyrimidin-9-yl)ethyl)amino)benzoic acid;
(R)-2-((1-(3-cyano-7-methyl-2-(4-(oxetan-3-yl) piperazin-1-yl)-4-oxo-4H-pyrido[1,2-a]pyrimidin-9-yl)ethyl)amino)benzoic acid;
(R)-2-((1-(3,7-dimethyl-2-(4-(oxetan-3-yl) piperazin-1-yl)-4-oxo-4H-pyrido[1,2-a]pyrimidin-9-yl)ethyl)amino)benzoic acid;
(R)-2-((1-(3-cyano-7-methyl-4-oxo-2-(4-(2,2,2-trifluoroethyl) piperazin-1-yl)-4H-pyrido[1,2-a]pyrimidin-9-yl)ethyl)amino)benzoic acid;
(R)-2-((1-(3-cyano-2-(((1-fluorocyclopropyl)methyl)amino)-7-methyl-4-oxo-4H-pyrido[1,2-a]pyrimidin-9-yl)ethyl)amino)benzoic acid;
2-(((R)-1-(3-cyano-2-((((R)-2,2-difluorocyclopropyl)methyl)amino)-7-methyl-4-oxo-4H-pyrido[1,2-a]pyrimidin-9-yl)ethyl)amino)benzoic acid;
2-(((R)-1-(3-cyano-2-((((S)-2,2-difluorocyclopropyl)methyl)amino)-7-methyl-4-oxo-4H-pyrido[1,2-a]pyrimidin-9-yl)ethyl)amino)benzoic acid;
2-(((1R)-1-(3-cyano-2-(((2,2-difluorocyclopropyl)methyl)amino)-7-methyl-4-oxo-4H-pyrido[1,2-a]pyrimidin-9-yl)ethyl)amino)benzoic acid;
(R)-2-((1-(3-cyano-7-methyl-4-oxo-2-(((1-(trifluoromethyl)cyclopropyl)methyl)amino)-4H-pyrido[1,2-a]pyrimidin-9-yl)ethyl)amino)benzoic acid;
2-(((R)-1-(3-cyano-2-(((R)-1-cyclopropyl-2,2,2-trifluoroethyl)amino)-7-methyl-4-oxo-4H-pyrido[1,2-a]pyrimidin-9-yl)ethyl)amino)benzoic acid;
2-(((R)-1-(3-cyano-2-(((S)-1-cyclopropyl-2,2,2-trifluoroethyl)amino)-7-methyl-4-oxo-4H-pyrido[1,2-a]pyrimidin-9-yl)ethyl)amino)benzoic acid;
2-(((R)-1-(2-(((R)-1-cyclopropyl-2,2,2-trifluoroethyl)amino)-3,7-dimethyl-4-oxo-4H-pyrido[1,2-a]pyrimidin-9-yl)ethyl)amino)benzoic acid;
2-(((R)-1-(2-(((S)-1-cyclopropyl-2,2,2-trifluoroethyl)amino)-3,7-dimethyl-4-oxo-4H-pyrido[1,2-a]pyrimidin-9-yl)ethyl)amino)benzoic acid;
2-(((R)-1-(3-cyano-2-(((S)-1-cyclopropylethyl)amino)-7-methyl-4-oxo-4H-pyrido[1,2-a]pyrimidin-9-yl)ethyl)amino)benzoic acid;
2-(((R)-1-(3-cyano-2-(((R)-1-cyclopropylethyl)amino)-7-methyl-4-oxo-4H-pyrido[1,2-a]pyrimidin-9-yl)ethyl)amino)benzoic acid;
2-(((R)-1-(2-(((S)-1-cyclopropylethyl)amino)-3,7-dimethyl-4-oxo-4H-pyrido[1,2-a]pyrimidin-9-yl)ethyl)amino)benzoic acid;
2-(((R)-1-(2-(((R)-1-cyclopropylethyl)amino)-3,7-dimethyl-4-oxo-4H-pyrido[1,2-a]pyrimidin-9-yl)ethyl)amino)benzoic acid;
(R)-2-((1-(3-cyano-7-(difluoromethyl)-2-(4,4-difluoropiperidin-1-yl)-4-oxo-4H-pyrido[1,2-a]pyrimidin-9-yl)ethyl)amino)benzoic acid;
(S)-2-((1-(3-cyano-7-(difluoromethyl)-2-(4,4-difluoropiperidin-1-yl)-4-oxo-4H-pyrido[1,2-a]pyrimidin-9-yl)ethyl)amino)benzoic acid;
2-((1-(3-cyano-7-(difluoromethyl)-2-(4,4-difluoropiperidin-1-yl)-4-oxo-4H-pyrido[1,2-a]pyrimidin-9-yl)ethyl)amino)benzoic acid;
(R)-2-((1-(7-(difluoromethyl)-2-(4,4-difluoropiperidin-1-yl)-3-methyl-4-oxo-4H-pyrido[1,2-a]pyrimidin-9-yl)ethyl)amino)benzoic acid;
(R)-2-((1-(3-cyano-2-(7-(4-cyanophenyl)-2,7-diazaspiro[3.5]nonan-2-yl)-7-methyl-4-oxo-4H-pyrido[1,2-a]pyrimidin-9-yl)ethyl)amino)benzoic acid;
(R)-2-((1-(3-cyano-7-methyl-2-(4-(1-methyl-3-(trifluoromethyl)-1H-pyrazol-4-yl) piperazin-1-yl)-4-oxo-4H-pyrido[1,2-a]pyrimidin-9-yl)ethyl)amino)benzoic acid;
2-(((R)-1-(3-cyano-2-((1R,4R)-5-(4-cyanophenyl)-2,5-diazabicyclo[2.2.1]heptan-2-yl)-7-methyl-4-oxo-4H-pyrido[1,2-a]pyrimidin-9-yl)ethyl)amino)benzoic acid;
2-(((R)-1-(3-cyano-2-((1S,4S)-5-(4-cyanophenyl)-2,5-diazabicyclo[2.2.1]heptan-2-yl)-7-methyl-4-oxo-4H-pyrido[1,2-a]pyrimidin-9-yl)ethyl)amino)benzoic acid;
2-(((R)-1-(2-((1R,4R)-5-(4-cyanophenyl)-2,5-diazabicyclo[2.2.1]heptan-2-yl)-3,7-dimethyl-4-oxo-4H-pyrido[1,2-a]pyrimidin-9-yl)ethyl)amino) benzoic acid;
2-(((R)-1-(2-((1S,4S)-5-(4-cyanophenyl)-2,5-diazabicyclo[2.2.1]heptan-2-yl)-3,7-dimethyl-4-oxo-4H-pyrido[1,2-a]pyrimidin-9-yl)ethyl)amino) benzoic acid;
2-(((1R)-1-(2-(3,6-diazabicyclo[3.1.1]heptan-6-yl)-3-cyano-7-methyl-4-oxo-4H-pyrido[1,2-a]pyrimidin-9-yl)ethyl)amino)benzoic acid;
2-(((1R)-1-(3-cyano-2-(3-(4-cyanophenyl)-3,6-diazabicyclo[3.1.1]heptan-6-yl)-7-methyl-4-oxo-4H-pyrido[1,2-a]pyrimidin-9-yl)ethyl)amino)benzoic acid;
2-(((1R)-1-(2-(3,6-diazabicyclo[3.1.1]heptan-6-yl)-3,7-dimethyl-4-oxo-4H-pyrido[1,2-a]pyrimidin-9-yl)ethyl)amino)benzoic acid;
2-(((1R)-1-(2-(3-(4-cyanophenyl)-3,6-diazabicyclo[3.1.1]heptan-6-yl)-3,7-dimethyl-4-oxo-4H-pyrido[1,2-a]pyrimidin-9-yl)ethyl)amino)benzoic acid;
2-(((1R)-1-(3-cyano-2-(3-(4-cyanophenyl)-3,8-diazabicyclo[3.2.1]octan-8-yl)-7-methyl-4-oxo-4H-pyrido[1,2-a]pyrimidin-9-yl)ethyl)amino)benzoic acid;
2-(((1R)-1-(2-(3-(4-cyanophenyl)-3,8-diazabicyclo[3.2.1]octan-8-yl)-3,7-dimethyl-4-oxo-4H-pyrido[1,2-a]pyrimidin-9-yl)ethyl)amino)benzoic acid;
(R)-2-((1-(3-cyano-2-(4-(3-cyano-1-methyl-1H-pyrazol-4-yl) piperazin-1-yl)-7-methyl-4-oxo-4H-pyrido[1,2-a]pyrimidin-9-yl)ethyl)amino)benzoic acid;
(R)-2-((1-(2-(4-(3-cyano-1-methyl-1H-pyrazol-4-yl) piperazin-1-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(4-(tetrahydro-2H-pyran-4-yl) piperazin-1-yl)-4H-pyrido[1,2-a]pyrimidin-9-yl)ethyl)amino)benzoic acid;
(R)-2-((1-(3,7-dimethyl-4-oxo-2-(4-(tetrahydro-2H-pyran-4-yl) piperazin-1-yl)-4H-pyrido[1,2-a]pyrimidin-9-yl)ethyl)amino)benzoic acid;
(R)-2-((1-(2-(5,6-difluoroisoindolin-2-yl)-3,7-dimethyl-4-oxo-4H-pyrido[1,2-a]pyrimidin-9-yl)ethyl)amino)benzoic acid;
2-(((1R)-1-(2-(3,3-difluoro-8-azabicyclo[3.2.1]octan-8-yl)-3,7-dimethyl-4-oxo-4H-pyrido[1,2-a]pyrimidin-9-yl)ethyl)amino)benzoic acid;
(R)-2-((1-(3-cyano-2-(6-(4-cyanophenyl)-2,6-diazaspiro[3.3]heptan-2-yl)-7-methyl-4-oxo-4H-pyrido[1,2-a]pyrimidin-9-yl)ethyl)amino)benzoic acid;
(R)-2-((1-(2-(6-(4-cyanophenyl)-2,6-diazaspiro[3.3]heptan-2-yl)-3,7-dimethyl-4-oxo-4H-pyrido[1,2-a]pyrimidin-9-yl)ethyl)amino)benzoic acid;
2-(((R)-1-(3-cyano-7-methyl-4-oxo-2-((((S)-tetrahydrofuran-3-yl)methyl)amino)-4H-pyrido[1,2-a]pyrimidin-9-yl)ethyl)amino)benzoic acid;
2-(((R)-1-(3-cyano-7-methyl-4-oxo-2-((((R)-tetrahydrofuran-3-yl)methyl)amino)-4H-pyrido[1,2-a]pyrimidin-9-yl)ethyl)amino)benzoic acid;
2-(((1R)-1-(3-cyano-7-methyl-4-oxo-2-(((tetrahydrofuran-3-yl)methyl)amino)-4H-pyrido[1,2-a]pyrimidin-9-yl)ethyl)amino)benzoic acid;
2-(((R)-1-(3,7-dimethyl-4-oxo-2-((((S)-tetrahydrofuran-3-yl)methyl)amino)-4H-pyrido[1,2-a]pyrimidin-9-yl)ethyl)amino)benzoic acid;
2-(((R)-1-(3,7-dimethyl-4-oxo-2-((((R)-tetrahydrofuran-3-yl)methyl)amino)-4H-pyrido[1,2-a]pyrimidin-9-yl)ethyl)amino)benzoic acid;
2-(((1R)-1-(3,7-dimethyl-4-oxo-2-(((tetrahydrofuran-3-yl)methyl)amino)-4H-pyrido[1,2-a]pyrimidin-9-yl)ethyl)amino)benzoic acid;
(R)-2-((1-(3-cyano-2-(((3,3-difluorocyclobutyl)methyl)amino)-7-methyl-4-oxo-4H-pyrido[1,2-a]pyrimidin-9-yl)ethyl)amino)benzoic acid;
(R)-2-((1-(2-(((3,3-difluorocyclobutyl)methyl)amino)-3,7-dimethyl-4-oxo-4H-pyrido[1,2-a]pyrimidin-9-yl)ethyl)amino)benzoic acid;
(R)-2-((1-(3-cyano-2-(5,5-difluoro-2-azaspiro[3.3]heptan-2-yl)-7-methyl-4-oxo-4H-pyrido[1,2-a]pyrimidin-9-yl)ethyl)amino)benzoic acid;
(R)-2-((1-(2-(5,5-difluoro-2-azaspiro[3.3]heptan-2-yl)-3,7-dimethyl-4-oxo-4H-pyrido[1,2-a]pyrimidin-9-yl)ethyl)amino)benzoic acid;
(R)-2-((1-(2-(6,6-difluoro-2-azaspiro[3.3]heptan-2-yl)-3,7-dimethyl-4-oxo-4H-pyrido[1,2-a]pyrimidin-9-yl)ethyl)amino)benzoic acid;
(R)-2-((1-(2-((cyclopropylmethyl)(methyl)amino)-3,7-dimethyl-4-oxo-4H-pyrido[1,2-a]pyrimidin-9-yl)ethyl)amino)benzoic acid;
(R)-2-((1-(3-cyano-7-methyl-2-((oxetan-3-ylmethyl)amino)-4-oxo-4H-pyrido[1,2-a]pyrimidin-9-yl)ethyl)amino)benzoic acid;
(R)-2-((1-(3,7-dimethyl-2-((oxetan-3-ylmethyl)amino)-4-oxo-4H-pyrido[1,2-a]pyrimidin-9-yl)ethyl)amino)benzoic acid;
(R)-6-chloro-3-((1-(3-cyano-2-((cyclopropylmethyl)amino)-7-methyl-4-oxo-4H-pyrido[1,2-a]pyrimidin-9-yl)ethyl)amino) picolinic acid;
(R)-2-((1-(2-((cyclopropylmethyl)amino)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(2-oxa-7-azaspiro[3.5]nonan-7-yl)-4H-pyrido[1,2-a]pyrimidin-9-yl)ethyl)amino)benzoic acid;
2-(((R)-1-(3-cyano-7-methyl-4-oxo-2-((R)-3-(trifluoromethyl) piperidin-1-yl)-4H-pyrido[1,2-a]pyrimidin-9-yl)ethyl)amino)benzoic acid;
2-(((R)-1-(3-cyano-7-methyl-4-oxo-2-((S)-3-(trifluoromethyl) piperidin-1-yl)-4H-pyrido[1,2-a]pyrimidin-9-yl)ethyl)amino)benzoic acid;
2-(((R)-1-(3,7-dimethyl-4-oxo-2-((R)-3-(trifluoromethyl) piperidin-1-yl)-4H-pyrido[1,2-a]pyrimidin-9-yl)ethyl)amino)benzoic acid;
2-(((R)-1-(3,7-dimethyl-4-oxo-2-((S)-3-(trifluoromethyl) piperidin-1-yl)-4H-pyrido[1,2-a]pyrimidin-9-yl)ethyl)amino)benzoic acid;
(R)-2-((1-(3,7-dimethyl-4-oxo-2-(1,1,2,2-tetrafluoro-6-azaspiro[2.5]octan-6-yl)-4H-pyrido[1,2-a]pyrimidin-9-yl)ethyl)amino) benzoic acid;
2-(((1R)-1-(3-cyano-7-methyl-4-oxo-2-(1,2,3,4-tetrahydro-1,4-epiminonaphthalen-9-yl)-4H-pyrido[1,2-a]pyrimidin-9-yl)ethyl)amino) benzoic acid;
2-(((1R)-1-(3,7-dimethyl-4-oxo-2-(1,2,3,4-tetrahydro-1,4-epiminonaphthalen-9-yl)-4H-pyrido[1,2-a]pyrimidin-9-yl)ethyl)amino) benzoic acid;
(R)-2-((1-(3-cyano-7-methyl-4-oxo-2-(3-(trifluoromethyl)-5,6-dihydro-[1,2,4]triazolo[4,3-a]pyrazin-7(8H)-yl)-4H-pyrido[1,2-a]pyrimidin-9-yl)ethyl)amino)benzoic acid;
(R)-2-((1-(3,7-dimethyl-4-oxo-2-(3-(trifluoromethyl)-5,6-dihydro-[1,2,4]triazolo[4,3-a]pyrazin-7(8H)-yl)-4H-pyrido[1,2-a]pyrimidin-9-yl)ethyl)amino)benzoic acid;
(R)-2-((1-(3-cyano-2-(1,1-difluoro-6-azaspiro[2.5]octan-6-yl)-7-methyl-4-oxo-4H-pyrido[1,2-a]pyrimidin-9-yl)ethyl)amino)benzoic acid;
(R)-2-((1-(2-(1,1-difluoro-6-azaspiro[2.5]octan-6-yl)-3,7-dimethyl-4-oxo-4H-pyrido[1,2-a]pyrimidin-9-yl)ethyl)amino)benzoic acid;
(R)-2-((1-(3-cyano-7-methyl-4-oxo-2-(6-azaspiro[2.5]octan-6-yl)-4H-pyrido[1,2-a]pyrimidin-9-yl)ethyl)amino)benzoic acid;
(R)-2-((1-(3,7-dimethyl-4-oxo-2-(6-azaspiro[2.5]octan-6-yl)-4H-pyrido[1,2-a]pyrimidin-9-yl)ethyl)amino)benzoic acid;
2-(((1R)-1-(2-(6,6-difluoro-3-azabicyclo[3.1.0]hexan-3-yl)-3,7-dimethyl-4-oxo-4H-pyrido[1,2-a]pyrimidin-9-yl)ethyl)amino)benzoic acid;
2-(((1R)-1-(2-(3-azabicyclo[3.1.0]hexan-3-yl)-3-cyano-7-methyl-4-oxo-4H-pyrido[1,2-a]pyrimidin-9-yl)ethyl)amino)benzoic acid;
2-(((1R)-1-(2-(3-azabicyclo[3.1.0]hexan-3-yl)-3,7-dimethyl-4-oxo-4H-pyrido[1,2-a]pyrimidin-9-yl)ethyl)amino)benzoic acid;
(R)-2-((1-(3-cyano-2-(4-(3-cyanophenyl) piperazin-1-yl)-7-methyl-4-oxo-4H-pyrido[1,2-a]pyrimidin-9-yl)ethyl)amino)benzoic acid;
(R)-2-((1-(2-(4-(3-cyanophenyl) piperazin-1-yl)-3,7-dimethyl-4-oxo-4H-pyrido[1,2-a]pyrimidin-9-yl)ethyl)amino)benzoic acid;
(R)-2-((1-(2-(3-azabicyclo[3.1.1]heptan-3-yl)-3-cyano-7-methyl-4-oxo-4H-pyrido[1,2-a]pyrimidin-9-yl)ethyl)amino)benzoic acid;
2-(((R)-1-(2-((1R,4S)-6,6-difluoro-2-azabicyclo[2.2.1]heptan-2-yl)-3,7-dimethyl-4-oxo-4H-pyrido[1,2-a]pyrimidin-9-yl)ethyl)amino)benzoic acid;
6-chloro-3-(((1R)-1-(2-(6,6-difluoro-2-azabicyclo[2.2.1]heptan-2-yl)-3,7-dimethyl-4-oxo-4H-pyrido[1,2-a]pyrimidin-9-yl)ethyl)amino) picolinic acid;
2-(((R)-1-(3-cyano-2-((1R,4R)-5,5-difluoro-2-azabicyclo[2.2.1]heptan-2-yl)-7-methyl-4-oxo-4H-pyrido[1,2-a]pyrimidin-9-yl)ethyl)amino)benzoic acid;
2-(((R)-1-(2-((1S,4S)-5,5-difluoro-2-azabicyclo[2.2.1]heptan-2-yl)-3,7-dimethyl-4-oxo-4H-pyrido[1,2-a]pyrimidin-9-yl)ethyl)amino)benzoic acid;
(R)-5-ethynyl-2-((1-(7-methyl-4-oxo-2-(piperidin-1-yl)-4H-pyrido[1,2-a]pyrimidin-9-yl)ethyl)amino)benzoic acid;
(R)-2-((1-(2-(1-(4-cyanophenyl)-1H-pyrazol-4-yl)-3,7-dimethyl-4-oxo-4H-pyrido[1,2-a]pyrimidin-9-yl)ethyl)amino)benzoic acid;
(R)-2-((1-(3,7-dimethyl-2-(1-methyl-1H-imidazol-4-yl)-4-oxo-4H-pyrido[1,2-a]pyrimidin-9-yl)ethyl)amino)benzoic acid;
(R)-2-((1-(2-(1H-imidazol-4-yl)-3,7-dimethyl-4-oxo-4H-pyrido[1,2-a]pyrimidin-9-yl)ethyl)amino)benzoic acid;
(R)-2-((1-(2-(1-(4-cyanophenyl)-1H-imidazol-4-yl)-3,7-dimethyl-4-oxo-4H-pyrido[1,2-a]pyrimidin-9-yl)ethyl)amino)benzoic acid;
(R)-2-((1-(2-(1-(4-cyanophenyl)-1H-imidazol-4-yl)-7-methyl-4-oxo-4H-pyrido[1,2-a]pyrimidin-9-yl)ethyl)amino)benzoic acid;
(R)-2-((1-(2-(3-(2-cyanopropan-2-yl)phenyl)-3,7-dimethyl-4-oxo-4H-pyrido[1,2-a]pyrimidin-9-yl)ethyl)amino)benzoic acid;
(R)-2-((1-(2-(3-(2-cyanopropan-2-yl)phenyl)-7-methyl-4-oxo-4H-pyrido[1,2-a]pyrimidin-9-yl)ethyl)amino)benzoic acid;
(R)-2-((1-(2-(1-(4-cyanophenyl)-1H-pyrazol-4-yl)-7-methyl-4-oxo-4H-pyrido[1,2-a]pyrimidin-9-yl)ethyl)amino)benzoic acid;
2-((1-(2-(4,4-difluoropiperidin-1-yl)-3,7-dimethyl-4-oxo-4H-pyrido[1,2-a]pyrimidin-9-yl) prop-2-yn-1-yl)amino)benzoic acid;
(R)-2-((1-(2-chloro-3,7-dimethyl-4-oxo-4H-pyrido[1,2-a]pyrimidin-9-yl)ethyl)amino)benzoic acid;
(R)-2-((1-(2-chloro-3-cyano-7-methyl-4-oxo-4H-pyrido[1,2-a]pyrimidin-9-yl)ethyl)amino)benzoic acid;
(R)-2-((1-(3-cyano-2-cyclopropyl-7-methyl-4-oxo-4H-pyrido[1,2-a]pyrimidin-9-yl)ethyl)amino)benzoic acid;
2-(((R)-1-(3,7-dimethyl-4-oxo-2-(((R)-tetrahydrofuran-3-yl)oxy)-4H-pyrido[1,2-a]pyrimidin-9-yl)ethyl)amino)benzoic acid;
2-(((R)-1-(3,7-dimethyl-4-oxo-2-(((S)-tetrahydrofuran-3-yl)oxy)-4H-pyrido[1,2-a]pyrimidin-9-yl)ethyl)amino)benzoic acid;
2-(((R)-1-(3-cyano-7-methyl-4-oxo-2-(((R)-tetrahydrofuran-3-yl)oxy)-4H-pyrido[1,2-a]pyrimidin-9-yl)ethyl)amino)benzoic acid;
2-(((R)-1-(3-cyano-7-methyl-4-oxo-2-(((S)-tetrahydrofuran-3-yl)oxy)-4H-pyrido[1,2-a]pyrimidin-9-yl)ethyl)amino)benzoic acid;
(R)-2-((1-(3,7-dimethyl-2-(4-(4-(methylcarbamoyl)phenyl) piperazin-1-yl)-4-oxo-4H-pyrido[1,2-a]pyrimidin-9-yl)ethyl)amino)benzoic acid;
(R)-2-((1-(2-(4-(4-carbamoylphenyl) piperazin-1-yl)-3,7-dimethyl-4-oxo-4H-pyrido[1,2-a]pyrimidin-9-yl)ethyl)amino)benzoic acid;
(R)-2-((1-(2-(4-(3-cyano-1-methyl-1H-indazol-7-yl) piperazin-1-yl)-3,7-dimethyl-4-oxo-4H-pyrido[1,2-a]pyrimidin-9-yl)ethyl)amino)benzoic acid;
(R)-2-((1-(2-(4-(1H-pyrazol-3-yl) piperidin-1-yl)-3,7-dimethyl-4-oxo-4H-pyrido[1,2-a]pyrimidin-9-yl)ethyl)amino)benzoic acid;
(R)-2-((1-(2-((4-cyanophenyl)ethynyl)-3,7-dimethyl-4-oxo-4H-pyrido[1,2-a]pyrimidin-9-yl)ethyl)amino)benzoic acid;
(R)-2-((1-(2-(4-(4-cyano-6-hydroxynaphthalen-1-yl) piperazin-1-yl)-3,7-dimethyl-4-oxo-4H-pyrido[1,2-a]pyrimidin-9-yl)ethyl)amino)benzoic acid;
(R)-2-((1-(2-(4-(4-cyano-7-hydroxynaphthalen-1-yl) piperazin-1-yl)-3,7-dimethyl-4-oxo-4H-pyrido[1,2-a]pyrimidin-9-yl)ethyl)amino)benzoic acid;
(R)-2-((1-(2-(1-acetylpiperidin-4-yl)-3-cyano-7-methyl-4-oxo-4H-pyrido[1,2-a]pyrimidin-9-yl)ethyl)amino)benzoic acid;
2-((1-(2-(4-(4-(aminomethyl)phenyl) piperazin-1-yl)-7-methyl-4-oxo-4H-pyrido[1,2-a]pyrimidin-9-yl)ethyl)amino)benzoic acid;
(R)-2-((1-(2-(4-(5-cyanopyrimidin-2-yl) piperazin-1-yl)-3,7-dimethyl-4-oxo-4H-pyrido[1,2-a]pyrimidin-9-yl)ethyl)amino)benzoic acid;
2-(((1R)-1-(3,7-dimethyl-4-oxo-2-(tetrahydro-1H-thieno[3,4-c]pyrrol-5(3H)-yl)-4H-pyrido[1,2-a]pyrimidin-9-yl)ethyl)amino)benzoic acid;
(R)-2-((1-(3-cyano-2-(4-(2-cyanophenyl) piperazin-1-yl)-7-methyl-4-oxo-4H-pyrido[1,2-a]pyrimidin-9-yl)ethyl)amino)benzoic acid;
(R)-2-((1-(3-cyano-7-methyl-2-(4-(1-methyl-1H-pyrazole-4-carbonyl) piperazin-1-yl)-4-oxo-4H-pyrido[1,2-a]pyrimidin-9-yl)ethyl)amino)benzoic acid;
(R)-2-((1-(3,7-dimethyl-2-(4-(1-methyl-1H-pyrazole-4-carbonyl) piperazin-1-yl)-4-oxo-4H-pyrido[1,2-a]pyrimidin-9-yl)ethyl)amino)benzoic acid;
(R)-2-((1-(2-(4-benzoylpiperazin-1-yl)-3-cyano-7-methyl-4-oxo-4H-pyrido[1,2-a]pyrimidin-9-yl)ethyl)amino)benzoic acid;
(R)-2-((1-(3-cyano-7-methyl-2-(4-(1-methyl-1H-pyrazol-4-yl) piperazin-1-yl)-4-oxo-4H-pyrido[1,2-a]pyrimidin-9-yl)ethyl)amino)benzoic acid;
(R)-2-((1-(3,7-dimethyl-2-(4-(1-methyl-1H-pyrazol-4-yl) piperazin-1-yl)-4-oxo-4H-pyrido[1,2-a]pyrimidin-9-yl)ethyl)amino)benzoic acid;
(R)-2-((1-(3-cyano-7-methyl-4-oxo-2-(4-(o-tolyl) piperazin-1-yl)-4H-pyrido[1,2-a]pyrimidin-9-yl)ethyl)amino)benzoic acid;
(R)-2-((1-(3,7-dimethyl-4-oxo-2-(4-(o-tolyl) piperazin-1-yl)-4H-pyrido[1,2-a]pyrimidin-9-yl)ethyl)amino)benzoic acid;
2-(((1R)-1-(3,7-dimethyl-2-(2-methyl-4-phenylpiperazin-1-yl)-4-oxo-4H-pyrido[1,2-a]pyrimidin-9-yl)ethyl)amino)benzoic acid;
2-(((1R)-1-(2-(4-(4-cyanophenyl)-2-methylpiperazin-1-yl)-3,7-dimethyl-4-oxo-4H-pyrido[1,2-a]pyrimidin-9-yl)ethyl)amino)benzoic acid;
2-(((R)-1-(3-cyano-2-((R)-4-(4-cyanophenyl)-3-methylpiperazin-1-yl)-7-methyl-4-oxo-4H-pyrido[1,2-a]pyrimidin-9-yl)ethyl)amino)benzoic acid;
2-(((1R)-1-(3-cyano-2-(4-(4-cyanophenyl)-3-methylpiperazin-1-yl)-7-methyl-4-oxo-4H-pyrido[1,2-a]pyrimidin-9-yl)ethyl)amino)benzoic acid;
(R)-2-((1-(3-cyano-2-(4-(4-cyanophenyl) piperazin-1-yl)-7-methyl-4-oxo-4H-pyrido[1,2-a]pyrimidin-9-yl)ethyl)amino)benzoic acid;
(R)-2-((1-(3,7-dimethyl-4-oxo-2-(4-phenylpiperazin-1-yl)-4H-pyrido[1,2-a]pyrimidin-9-yl)ethyl)amino)benzoic acid;
(R)-4-((1-(3-cyano-2-(isoindolin-2-yl)-7-methyl-4-oxo-4H-pyrido[1,2-a]pyrimidin-9-yl)ethyl)amino) nicotinic acid;
(R)-4-((1-(2-(isoindolin-2-yl)-3,7-dimethyl-4-oxo-4H-pyrido[1,2-a]pyrimidin-9-yl)ethyl)amino) nicotinic acid;
(R)-6-chloro-4-((1-(2-(isoindolin-2-yl)-3,7-dimethyl-4-oxo-4H-pyrido[1,2-a]pyrimidin-9-yl)ethyl)amino)pyridazine-3-carboxylic acid;
(R)-4-((1-(2-(isoindolin-2-yl)-3,7-dimethyl-4-oxo-4H-pyrido[1,2-a]pyrimidin-9-yl)ethyl)amino)pyridazine-3-carboxylic acid;
(R)-3,7-dimethyl-9-(1-((1-methyl-2-oxo-1,2-dihydropyridin-3-yl)amino)ethyl)-2-(piperidin-1-yl)-4H-pyrido[1,2-a]pyrimidin-4-one
(R)-6-chloro-3-((1-(2-(4,4-dimethylpiperidin-1-yl)-3,7-dimethyl-4-oxo-4H-pyrido[1,2-a]pyrimidin-9-yl)ethyl)amino) picolinic acid;
(R)-5-chloro-2-((1-(2-(isoindolin-2-yl)-3,7-dimethyl-4-oxo-4H-pyrido[1,2-a]pyrimidin-9-yl)ethyl)amino)benzoic acid;
(R)-5-chloro-2-((1-(3,7-dimethyl-4-oxo-2-(piperidin-1-yl)-4H-pyrido[1,2-a]pyrimidin-9-yl)ethyl)amino)benzoic acid;
(R)-2-((1-(3-cyano-2-((3-(hydroxymethyl) bicyclo[1.1.1]pentan-1-yl)amino)-7-methyl-4-oxo-4H-pyrido[1,2-a]pyrimidin-9-yl)ethyl)amino) benzoic acid;
(R)-2-((1-(3-cyano-7-methyl-2-(4-(methylsulfonyl) piperazin-1-yl)-4-oxo-4H-pyrido[1,2-a]pyrimidin-9-yl)ethyl)amino)benzoic acid;
(R)-2-((1-(3-cyano-7-methyl-4-oxo-2-(3-oxopiperazin-1-yl)-4H-pyrido[1,2-a]pyrimidin-9-yl)ethyl)amino)benzoic acid;
2-(((1R)-1-(3-cyano-7-methyl-4-oxo-2-(1-oxa-6-azaspiro[3.4]octan-6-yl)-4H-pyrido[1,2-a]pyrimidin-9-yl)ethyl)amino)benzoic acid;
(R)-2-((1-(3-cyano-2-(1,3-dihydro-2H-pyrrolo[3,4-c]pyridin-2-yl)-7-methyl-4-oxo-4H-pyrido[1,2-a]pyrimidin-9-yl)ethyl)amino)benzoic acid;
(R)-2-((1-(3-cyano-2-(5,7-dihydro-6H-pyrrolo[3,4-b]pyridin-6-yl)-7-methyl-4-oxo-4H-pyrido[1,2-a]pyrimidin-9-yl)ethyl)amino)benzoic acid;
2-(((1R)-1-(3-cyano-2-(1-(hydroxymethyl) isoindolin-2-yl)-7-methyl-4-oxo-4H-pyrido[1,2-a]pyrimidin-9-yl)ethyl)amino)benzoic acid;
2-(((1R)-1-(3-cyano-7-methyl-4-oxo-2-(2-oxo-1,7-diazaspiro[4.4]nonan-7-yl)-4H-pyrido[1,2-a]pyrimidin-9-yl)ethyl)amino)benzoic acid;
(R)-2-((1-(7-fluoro-2-(isoindolin-2-yl)-3-methyl-4-oxo-4H-pyrido[1,2-a]pyrimidin-9-yl)ethyl)amino)benzoic acid;
(R)-2-((1-(7-chloro-2-(isoindolin-2-yl)-3-methyl-4-oxo-4H-pyrido[1,2-a]pyrimidin-9-yl)ethyl)amino)benzoic acid;
(R)-5-((1-(3-cyano-2-(4,4-difluoropiperidin-1-yl)-7-methyl-4-oxo-4H-pyrido[1,2-a]pyrimidin-9-yl)ethyl)amino) thiazole-4-carboxylic acid;
(R)-2-((1-(3-cyano-7-methyl-4-oxo-2-(piperidin-1-yl)-4H-pyrido[1,2-a]pyrimidin-9-yl)ethyl)amino)-5-fluorobenzoic acid;
2-(((R)-1-(2-((1R,4S)-2-azabicyclo[2.2.1]heptan-2-yl)-3-cyano-7-methyl-4-oxo-4H-pyrido[1,2-a]pyrimidin-9-yl)ethyl)amino)benzoic acid;
(R)-2-((1-(3-cyano-7-methyl-2-(4-methylpiperazin-1-yl)-4-oxo-4H-pyrido[1,2-a]pyrimidin-9-yl)ethyl)amino)benzoic acid;
(R)-2-((1-(3-cyano-7-methyl-4-oxo-2-(piperazin-1-yl)-4H-pyrido[1,2-a]pyrimidin-9-yl)ethyl)amino)benzoic acid;
(R)-2-((1-(3-cyano-2-(3,3-difluoropiperidin-1-yl)-7-methyl-4-oxo-4H-pyrido[1,2-a]pyrimidin-9-yl)ethyl)amino)benzoic acid;
(R)-6-chloro-3-((1-(2-(4,4-difluoropiperidin-1-yl)-3,7-dimethyl-4-oxo-4H-pyrido[1,2-a]pyrimidin-9-yl)ethyl)amino) picolinic acid;
7-methyl-4-oxo-9-(1-(phenylamino)ethyl)-2-(piperidin-1-yl)-4H-pyrido[1,2-a]pyrimidine-3-carbonitrile
(S)-2-(isoindolin-2-yl)-7-methyl-4-oxo-9-(1-(phenylamino)ethyl)-4H-pyrido[1,2-a]pyrimidine-3-carbonitrile
(R)-2-(isoindolin-2-yl)-7-methyl-4-oxo-9-(1-(phenylamino)ethyl)-4H-pyrido[1,2-a]pyrimidine-3-carbonitrile
2-(isoindolin-2-yl)-7-methyl-4-oxo-9-(1-(phenylamino)ethyl)-4H-pyrido[1,2-a]pyrimidine-3-carbonitrile
(R)-2-((1-(2-(4,4-difluoropiperidin-1-yl)-3,7-dimethyl-4-oxo-4H-pyrido[1,2-a]pyrimidin-9-yl)ethyl)amino)benzoic acid;
(R)-2-((1-(2-(4,6-dihydro-5H-pyrrolo[3,4-d]thiazol-5-yl)-3,7-dimethyl-4-oxo-4H-pyrido[1,2-a]pyrimidin-9-yl)ethyl)amino)benzoic acid;
(R)-2-((1-(3-cyano-7-methyl-2-(2-methyl-6,7-dihydrothiazolo[4,5-c]pyridin-5(4H)-yl)-4-oxo-4H-pyrido[1,2-a]pyrimidin-9-yl)ethyl)amino) benzoic acid;
(R)-2-((1-(3-cyano-2-(6,7-dihydrothiazolo[4,5-c]pyridin-5(4H)-yl)-7-methyl-4-oxo-4H-pyrido[1,2-a]pyrimidin-9-yl)ethyl)amino)benzoic acid;
(R)-2-((1-(3-cyano-2-(isoindolin-2-yl)-7-methyl-4-oxo-4H-pyrido[1,2-a]pyrimidin-9-yl)ethyl)amino)-6-fluorobenzoic acid;
(R)-2-((1-(3-cyano-2-(isobutylamino)-7-methyl-4-oxo-4H-pyrido[1,2-a]pyrimidin-9-yl)ethyl)amino)benzoic acid;
(R)-2-((1-(2-(isobutylamino)-3,7-dimethyl-4-oxo-4H-pyrido[1,2-a]pyrimidin-9-yl)ethyl)amino)benzoic acid;
(R)-5-cyano-2-((1-(2-(isoindolin-2-yl)-3,7-dimethyl-4-oxo-4H-pyrido[1,2-a]pyrimidin-9-yl)ethyl)amino)benzoic acid;
(R)-2-((1-(2-(4-(dimethylamino) piperidin-1-yl)-3,7-dimethyl-4-oxo-4H-pyrido[1,2-a]pyrimidin-9-yl)ethyl)amino)benzoic acid;
(R)-2-((1-(3-cyano-2-(4-hydroxypiperidin-1-yl)-7-methyl-4-oxo-4H-pyrido[1,2-a]pyrimidin-9-yl)ethyl)amino)benzoic acid;
(R)-2-((1-(3-cyano-2-(4-(dimethylamino) piperidin-1-yl)-7-methyl-4-oxo-4H-pyrido[1,2-a]pyrimidin-9-yl)ethyl)amino)benzoic acid;
(R)-2-((1-(3-cyano-7-methyl-2-(4-methylpiperidin-1-yl)-4-oxo-4H-pyrido[1,2-a]pyrimidin-9-yl)ethyl)amino)benzoic acid;
2-(3-cyano-7-methyl-4-oxo-2-(piperidin-1-yl)-4H-pyrido[1,2-a]pyrimidine-9-carboxamido) cyclohexane-1-carboxylic acid;
2-(1-(3-cyano-7-methyl-4-oxo-2-(piperidin-1-yl)-4H-pyrido[1,2-a]pyrimidine-9-carboxamido) cyclobutyl)acetic acid;
(R)-1-((1-(3-cyano-7-methyl-4-oxo-2-(piperidin-1-yl)-4H-pyrido[1,2-a]pyrimidin-9-yl)ethyl) carbamoyl) cyclobutane-1-carboxylic acid;
(R)-2-((1-(3-cyano-7-methyl-2-morpholino-4-oxo-4H-pyrido[1,2-a]pyrimidin-9-yl)ethyl)amino)benzoic acid;
(R)-2-((1-(2-(azepan-1-yl)-3-cyano-7-methyl-4-oxo-4H-pyrido[1,2-a]pyrimidin-9-yl)ethyl)amino)benzoic acid;
(R)-2-((1-(3-cyano-2-(diethylamino)-7-methyl-4-oxo-4H-pyrido[1,2-a]pyrimidin-9-yl)ethyl)amino)benzoic acid;
(R)-2-((1-(3-cyano-7-methyl-4-oxo-2-(2-oxa-6-azaspiro[3.3]heptan-6-yl)-4H-pyrido[1,2-a]pyrimidin-9-yl)ethyl)amino)benzoic acid;
2-(((1R)-1-(2-(6-oxa-3-azabicyclo[3.1.1]heptan-3-yl)-3-cyano-7-methyl-4-oxo-4H-pyrido[1,2-a]pyrimidin-9-yl)ethyl)amino)benzoic acid;
(R)-2-((1-(3-cyano-7-methyl-4-oxo-2-(2-azaspiro[3.3]heptan-2-yl)-4H-pyrido[1,2-a]pyrimidin-9-yl)ethyl)amino)benzoic acid;
2-(((1R)-1-(3-cyano-2-(3-hydroxypiperidin-1-yl)-7-methyl-4-oxo-4H-pyrido[1,2-a]pyrimidin-9-yl)ethyl)amino)benzoic acid;
(R)-2-chloro-5-((1-(3-cyano-2-(isoindolin-2-yl)-7-methyl-4-oxo-4H-pyrido[1,2-a]pyrimidin-9-yl)ethyl)amino)pyrimidine-4-carboxylic acid;
(R)-2-((1-(3-cyano-7-cyclopropyl-2-(isoindolin-2-yl)-4-oxo-4H-pyrido[1,2-a]pyrimidin-9-yl)ethyl)amino)benzoic acid;
(S)-2-((1-(3-cyano-7-cyclopropyl-2-(isoindolin-2-yl)-4-oxo-4H-pyrido[1,2-a]pyrimidin-9-yl)ethyl)amino)benzoic acid;
(R)-2-((1-(3-cyano-7-ethyl-2-(isoindolin-2-yl)-4-oxo-4H-pyrido[1,2-a]pyrimidin-9-yl)ethyl)amino)benzoic acid;
(S)-1-(3-cyano-7-methyl-4-oxo-2-(piperidin-1-yl)-4H-pyrido[1,2-a]pyrimidin-9-yl)ethyl 3-amino-6-chloropicolinate
(R)-1-(3-cyano-7-methyl-4-oxo-2-(piperidin-1-yl)-4H-pyrido[1,2-a]pyrimidin-9-yl)ethyl 3-amino-6-chloropicolinate
(R)-6-chloro-3-((1-(3-cyano-7-methyl-4-oxo-2-(piperidin-1-yl)-4H-pyrido[1,2-a]pyrimidin-9-yl)ethyl)amino) picolinic acid;
6-chloro-3-((1-(3-cyano-7-methyl-4-oxo-2-(piperidin-1-yl)-4H-pyrido[1,2-a]pyrimidin-9-yl)ethyl)amino) picolinic acid;
(2-((1-(2-(isoindolin-2-yl)-7-methyl-4-oxo-4H-pyrido[1,2-a]pyrimidin-9-yl)ethyl)amino)phenyl)(methyl) phosphinic acid;
2-((1-(2-(4-(4-cyanophenyl) piperazin-1-yl)-7-methyl-4-oxo-4H-pyrido[1,2-a]pyrimidin-9-yl)ethyl)amino)benzoic acid;
2-((1-(7-methyl-4-oxo-2-(4-phenylpiperazin-1-yl)-4H-pyrido[1,2-a]pyrimidin-9-yl)ethyl)amino)benzoic acid;
2-((1-(2-(4-(cyclopropanecarbonyl) piperazin-1-yl)-7-methyl-4-oxo-4H-pyrido[1,2-a]pyrimidin-9-yl)ethyl)amino)benzoic acid;
2-((1-(2-(4-acetylpiperazin-1-yl)-7-methyl-4-oxo-4H-pyrido[1,2-a]pyrimidin-9-yl)ethyl)amino)benzoic acid;
2-((1-(2-(4-cyanoisoindolin-2-yl)-7-methyl-4-oxo-4H-pyrido[1,2-a]pyrimidin-9-yl)ethyl)amino)benzoic acid;
2-((1-(2-((3-cyanophenyl)amino)-7-methyl-4-oxo-4H-pyrido[1,2-a]pyrimidin-9-yl)ethyl)amino)benzoic acid;
2-((1-(2-(3-cyanophenoxy)-7-methyl-4-oxo-4H-pyrido[1,2-a]pyrimidin-9-yl)ethyl)amino)benzoic acid;
2-((1-(7-methyl-4-oxo-2-phenoxy-4H-pyrido[1,2-a]pyrimidin-9-yl)ethyl)amino)benzoic acid;
2-((1-(2-(3-hydroxy-3-methylpiperidin-1-yl)-7-methyl-4-oxo-4H-pyrido[1,2-a]pyrimidin-9-yl)ethyl)amino)benzoic acid;
2-((1-(2-(3-hydroxypiperidin-1-yl)-7-methyl-4-oxo-4H-pyrido[1,2-a]pyrimidin-9-yl)ethyl)amino)benzoic acid;
2-((1-(2-(7-azabicyclo[2.2.1]heptan-7-yl)-7-methyl-4-oxo-4H-pyrido[1,2-a]pyrimidin-9-yl)ethyl)amino)benzoic acid;
(7-methyl-4-oxo-2-(piperidin-1-yl)-4H-pyrido[1,2-a]pyrimidine-9-carbonyl) proline
1-(7-methyl-4-oxo-2-(piperidin-1-yl)-4H-pyrido[1,2-a]pyrimidine-9-carbonyl) piperidine-2-carboxylic acid;
1-(7-methyl-4-oxo-2-(piperidin-1-yl)-4H-pyrido[1,2-a]pyrimidin-9-yl) piperidine-4-carboxylic acid;
2-chloro-5-((1-(2-(isoindolin-2-yl)-3,7-dimethyl-4-oxo-4H-pyrido[1,2-a]pyrimidin-9-yl)ethyl)amino) isonicotinic acid;
2-((1-(3-cyano-2-(isoindolin-2-yl)-7-methyl-4-oxo-4H-pyrido[1,2-a]pyrimidin-9-yl)ethyl)amino)benzoic acid;
4-((1-(2-(isoindolin-2-yl)-3,7-dimethyl-4-oxo-4H-pyrido[1,2-a]pyrimidin-9-yl)ethyl)amino) nicotinic acid;
(R)-6-fluoro-3-((1-(2-(isoindolin-2-yl)-3,7-dimethyl-4-oxo-4H-pyrido[1,2-a]pyrimidin-9-yl)ethyl)amino) picolinic acid;
6-fluoro-3-((1-(2-(isoindolin-2-yl)-3,7-dimethyl-4-oxo-4H-pyrido[1,2-a]pyrimidin-9-yl)ethyl)amino) picolinic acid;
(S)-3-((1-(2-(isoindolin-2-yl)-3,7-dimethyl-4-oxo-4H-pyrido[1,2-a]pyrimidin-9-yl)ethyl)amino)-6-(trifluoromethyl) picolinic acid;
(R)-3-((1-(2-(isoindolin-2-yl)-3,7-dimethyl-4-oxo-4H-pyrido[1,2-a]pyrimidin-9-yl)ethyl)amino)-6-(trifluoromethyl) picolinic acid;
3-((1-(2-(isoindolin-2-yl)-3,7-dimethyl-4-oxo-4H-pyrido[1,2-a]pyrimidin-9-yl)ethyl)amino)-6-(trifluoromethyl) picolinic acid;
3-((1-(3,7-dimethyl-4-oxo-2-(piperidin-1-yl)-4H-pyrido[1,2-a]pyrimidin-9-yl)ethyl)amino) picolinic acid;
(S)-6-chloro-3-((1-(3,7-dimethyl-4-oxo-2-(piperidin-1-yl)-4H-pyrido[1,2-a]pyrimidin-9-yl)ethyl)amino) picolinic acid;
(R)-6-chloro-3-((1-(3,7-dimethyl-4-oxo-2-(piperidin-1-yl)-4H-pyrido[1,2-a]pyrimidin-9-yl)ethyl)amino) picolinic acid;
6-chloro-3-((1-(3,7-dimethyl-4-oxo-2-(piperidin-1-yl)-4H-pyrido[1,2-a]pyrimidin-9-yl)ethyl)amino) picolinic acid;
(R)-6-chloro-3-((1-(2-(isoindolin-2-yl)-3,7-dimethyl-4-oxo-4H-pyrido[1,2-a]pyrimidin-9-yl)ethyl)amino) picolinic acid;
6-chloro-3-((1-(2-(isoindolin-2-yl)-3,7-dimethyl-4-oxo-4H-pyrido[1,2-a]pyrimidin-9-yl)ethyl)amino) picolinic acid;
2-((1-(3,7-dimethyl-4-oxo-2-(1-oxoisoindolin-2-yl)-4H-pyrido[1,2-a]pyrimidin-9-yl)ethyl)amino)benzoic acid;
3-fluoro-2-((1-(2-(isoindolin-2-yl)-3,7-dimethyl-4-oxo-4H-pyrido[1,2-a]pyrimidin-9-yl)ethyl)amino)benzoic acid;
4-fluoro-2-((1-(2-(isoindolin-2-yl)-3,7-dimethyl-4-oxo-4H-pyrido[1,2-a]pyrimidin-9-yl)ethyl)amino)benzoic acid;
(S)-5-fluoro-2-((1-(2-(isoindolin-2-yl)-3,7-dimethyl-4-oxo-4H-pyrido[1,2-a]pyrimidin-9-yl)ethyl)amino)benzoic acid;
(R)-5-fluoro-2-((1-(2-(isoindolin-2-yl)-3,7-dimethyl-4-oxo-4H-pyrido[1,2-a]pyrimidin-9-yl)ethyl)amino)benzoic acid;
5-fluoro-2-((1-(2-(isoindolin-2-yl)-3,7-dimethyl-4-oxo-4H-pyrido[1,2-a]pyrimidin-9-yl)ethyl)amino)benzoic acid;
2-fluoro-6-((1-(2-(isoindolin-2-yl)-3,7-dimethyl-4-oxo-4H-pyrido[1,2-a]pyrimidin-9-yl)ethyl)amino)benzoic acid;
2-((1-(7-methyl-4-oxo-2-((tetrahydrofuran-3-yl) thio)-4H-pyrido[1,2-a]pyrimidin-9-yl)ethyl)amino)benzoic acid;
2-((1-(7-methyl-4-oxo-2-((tetrahydrofuran-3-yl)oxy)-4H-pyrido[1,2-a]pyrimidin-9-yl)ethyl)amino)benzoic acid;
2-((1-(2-methoxy-7-methyl-4-oxo-4H-pyrido[1,2-a]pyrimidin-9-yl)ethyl)amino)benzoic acid;
2-((1-(7-methyl-4-oxo-2-(1H-pyrazol-4-yl)-4H-pyrido[1,2-a]pyrimidin-9-yl)ethyl)amino)benzoic acid;
2-(((S)-1-(7-methyl-2-((S)-1-methyl-2-oxopiperidin-4-yl)-4-oxo-4H-pyrido[1,2-a]pyrimidin-9-yl)ethyl)amino)benzoic acid;
2-(((S)-1-(7-methyl-2-((R)-1-methyl-2-oxopiperidin-4-yl)-4-oxo-4H-pyrido[1,2-a]pyrimidin-9-yl)ethyl)amino)benzoic acid;
2-(((R)-1-(7-methyl-2-((S)-1-methyl-2-oxopiperidin-4-yl)-4-oxo-4H-pyrido[1,2-a]pyrimidin-9-yl)ethyl)amino)benzoic acid;
2-((1-(7-methyl-2-(1-methyl-2-oxopiperidin-4-yl)-4-oxo-4H-pyrido[1,2-a]pyrimidin-9-yl)ethyl)amino)benzoic acid;
2-((1-(7-methyl-4-oxo-2-(1,2,3,6-tetrahydropyridin-4-yl)-4H-pyrido[1,2-a]pyrimidin-9-yl)ethyl)amino)benzoic acid;
(R)-2-((1-(2-(1-acetyl-1,2,3,6-tetrahydropyridin-4-yl)-7-methyl-4-oxo-4H-pyrido[1,2-a]pyrimidin-9-yl)ethyl)amino)benzoic acid;
2-((1-(7-methyl-4-oxo-2-(piperidin-4-yl)-4H-pyrido[1,2-a]pyrimidin-9-yl)ethyl)amino)benzoic acid;
2-((1-(2-(1-acetylpiperidin-4-yl)-7-methyl-4-oxo-4H-pyrido[1,2-a]pyrimidin-9-yl)ethyl)amino)benzoic acid;
2-((1-(7-methyl-2-(1-methyl-1,4,5,7-tetrahydro-6H-pyrazolo[3,4-c]pyridin-6-yl)-4-oxo-4H-pyrido[1,2-a]pyrimidin-9-yl)ethyl)amino)benzoic acid;
2-((1-(7-methyl-2-(1-methyl-6-oxo-1,6-dihydropyridin-3-yl)-4-oxo-4H-pyrido[1,2-a]pyrimidin-9-yl)ethyl)amino)benzoic acid;
2-((1-(7-methyl-2-(4-methyl-3-oxopiperazin-1-yl)-4-oxo-4H-pyrido[1,2-a]pyrimidin-9-yl)ethyl)amino)benzoic acid;
3-((1-(7-methyl-4-oxo-2-(piperidin-1-yl)-4H-pyrido[1,2-a]pyrimidin-9-yl)ethyl)amino)cyclobutane-1-carboxylic acid;
4-((1-(7-methyl-4-oxo-2-(piperidin-1-yl)-4H-pyrido[1,2-a]pyrimidin-9-yl)ethyl)amino)benzoic acid;
3-((1-(7-methyl-4-oxo-2-(piperidin-1-yl)-4H-pyrido[1,2-a]pyrimidin-9-yl)ethyl)amino)benzoic acid;
2-methyl-6-((1-(7-methyl-4-oxo-2-(piperidin-1-yl)-4H-pyrido[1,2-a]pyrimidin-9-yl)ethyl)amino)benzoic acid;
2-((1-(2-(4-fluorophenyl)-7-methyl-4-oxo-4H-pyrido[1,2-a]pyrimidin-9-yl)ethyl)amino)benzoic acid;
2-((1-(2-(4-methoxyphenyl)-7-methyl-4-oxo-4H-pyrido[1,2-a]pyrimidin-9-yl)ethyl)amino)benzoic acid;
2-((1-(2-(3-methoxyphenyl)-7-methyl-4-oxo-4H-pyrido[1,2-a]pyrimidin-9-yl)ethyl)amino)benzoic acid;
2-((1-(2-(2-methoxyphenyl)-7-methyl-4-oxo-4H-pyrido[1,2-a]pyrimidin-9-yl)ethyl)amino)benzoic acid;
2-((1-(7-methyl-4-oxo-2-(o-tolyl)-4H-pyrido[1,2-a]pyrimidin-9-yl)ethyl)amino)benzoic acid;
2-((1-(2-(4-chlorophenyl)-7-methyl-4-oxo-4H-pyrido[1,2-a]pyrimidin-9-yl)ethyl)amino)benzoic acid;
2-((1-(2-(4-cyanophenyl)-7-methyl-4-oxo-4H-pyrido[1,2-a]pyrimidin-9-yl)ethyl)amino)benzoic acid;
2-((1-(2-(3-cyanophenyl)-7-methyl-4-oxo-4H-pyrido[1,2-a]pyrimidin-9-yl)ethyl)amino)benzoic acid;
2-((1-(2-(2-cyanophenyl)-7-methyl-4-oxo-4H-pyrido[1,2-a]pyrimidin-9-yl)ethyl)amino)benzoic acid;
2-((1-(7-methyl-4-oxo-2-(pyridin-4-yl)-4H-pyrido[1,2-a]pyrimidin-9-yl)ethyl)amino)benzoic acid;
2-((1-(7-methyl-4-oxo-2-(pyridin-3-yl)-4H-pyrido[1,2-a]pyrimidin-9-yl)ethyl)amino)benzoic acid;
2-((1-(7-methyl-4-oxo-2-(pyridin-2-yl)-4H-pyrido[1,2-a]pyrimidin-9-yl)ethyl)amino)benzoic acid;
2-((1-(3,7-dimethyl-4-oxo-2-phenyl-4H-pyrido[1,2-a]pyrimidin-9-yl)ethyl)amino)benzoic acid;
2-((1-(7-methyl-2-(1-methyl-1H-pyrazol-3-yl)-4-oxo-4H-pyrido[1,2-a]pyrimidin-9-yl)ethyl)amino)benzoic acid;
2-((1-(7-methyl-2-(1-methyl-1H-pyrazol-4-yl)-4-oxo-4H-pyrido[1,2-a]pyrimidin-9-yl)ethyl)amino)benzoic acid;
(S)-2-((1-(2-(isoindolin-2-yl)-7-methyl-4-oxo-4H-pyrido[1,2-a]pyrimidin-9-yl)ethyl)amino)benzoic acid;
2-((1-(2-(azetidin-1-yl)-7-methyl-4-oxo-4H-pyrido[1,2-a]pyrimidin-9-yl)ethyl)amino)benzoic acid;
2-((1-(2-(dimethylamino)-7-methyl-4-oxo-4H-pyrido[1,2-a]pyrimidin-9-yl)ethyl)amino)benzoic acid;
2-((1-(7-methyl-4-oxo-2-(piperidin-1-yl)-4H-pyrido[1,2-a]pyrimidin-9-yl) propyl)amino)benzoic acid;
2-(((7-methyl-4-oxo-2-(piperidin-1-yl)-4H-pyrido[1,2-a]pyrimidin-9-yl)methyl)amino)benzoic acid;
2-((1-(3-cyclopropyl-7-methyl-4-oxo-2-(piperidin-1-yl)-4H-pyrido[1,2-a]pyrimidin-9-yl)ethyl)amino)benzoic acid;
(S)-2-((1-(7-methyl-4-oxo-2-(piperidin-1-yl)-3-(trifluoromethyl)-4H-pyrido[1,2-a]pyrimidin-9-yl)ethyl)amino)benzoic acid;
2-((1-(7-methyl-4-oxo-2-(piperidin-1-yl)-3-(trifluoromethyl)-4H-pyrido[1,2-a]pyrimidin-9-yl)ethyl)amino)benzoic acid;
2-((1-(4-oxo-2-(piperidin-1-yl)-7-(trifluoromethyl)-4H-pyrido[1,2-a]pyrimidin-9-yl)ethyl)amino)benzoic acid;
(S)-2-((1-(7-cyano-2-(4,4-difluoropiperidin-1-yl)-4-oxo-4H-pyrido[1,2-a]pyrimidin-9-yl)ethyl)amino)benzoic acid;
(R)-2-((1-(7-cyano-2-(4,4-difluoropiperidin-1-yl)-4-oxo-4H-pyrido[1,2-a]pyrimidin-9-yl)ethyl)amino)benzoic acid;
2-((1-(7-cyano-2-(4,4-difluoropiperidin-1-yl)-4-oxo-4H-pyrido[1,2-a]pyrimidin-9-yl)ethyl)amino)benzoic acid;
2-((1-(7-methyl-2-morpholino-4-oxo-4H-pyrido[1,2-a]pyrimidin-9-yl)ethyl)amino)benzoic acid;
7-methyl-2-morpholino-9-(1-(phenylamino)ethyl)-4H-pyrido[1,2-a]pyrimidin-4-one
(R)-2-((1-(4-oxo-2-(piperidin-1-yl)-4H-pyrido[1,2-a]pyrimidin-9-yl)ethyl)amino)benzoic acid;
(S)-2-((1-(4-oxo-2-(piperidin-1-yl)-4H-pyrido[1,2-a]pyrimidin-9-yl)ethyl)amino)benzoic acid;
6-(2-(isoindolin-2-yl)-7-methyl-4-oxo-4H-pyrido[1,2-a]pyrimidin-9-yl)-6-azaspiro[3.4]octane-8-carboxylic acid;
1-(2-(isoindolin-2-yl)-7-methyl-4-oxo-4H-pyrido[1,2-a]pyrimidin-9-yl)azetidine-3-carboxylic acid;
2-(1-(2-(isoindolin-2-yl)-7-methyl-4-oxo-4H-pyrido[1,2-a]pyrimidin-9-yl) pyrrolidin-3-yl)acetic acid;
1-(2-(isoindolin-2-yl)-7-methyl-4-oxo-4H-pyrido[1,2-a]pyrimidin-9-yl)-4-methyl-5-oxopiperazine-2-carboxylic acid;
(3aR,5r,6aS)-2-(2-(isoindolin-2-yl)-7-methyl-4-oxo-4H-pyrido[1,2-a]pyrimidin-9-yl) octahydrocyclopenta[c]pyrrole-5-carboxylic acid;
(R)-2-((1-(2-(5,6-dihydroimidazo[1,2-a]pyrazin-7(8H)-yl)-3,7-dimethyl-4-oxo-4H-pyrido[1,2-a]pyrimidin-9-yl)ethyl)amino)benzoic acid;
(R)-2-((1-(2-(5,6-dihydroimidazo[1,5-a]pyrazin-7(8H)-yl)-3,7-dimethyl-4-oxo-4H-pyrido[1,2-a]pyrimidin-9-yl)ethyl)amino)benzoic acid;
(R)-2-((1-(2-(6,7-dihydropyrazolo[1,5-a]pyrazin-5(4H)-yl)-3,7-dimethyl-4-oxo-4H-pyrido[1,2-a]pyrimidin-9-yl)ethyl)amino)benzoic acid;
(R)-2-((1-(2-(4-(1H-pyrazol-1-yl) piperidin-1-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(2-(trifluoromethyl)-5,6-dihydroimidazo[1,2-a]pyrazin-7(8H)-yl)-4H-pyrido[1,2-a]pyrimidin-9-yl)ethyl)amino)benzoic acid;
(R)-2-((1-(3,7-dimethyl-4-oxo-2-(4-(5-(trifluoromethyl)pyridin-2-yl) piperazin-1-yl)-4H-pyrido[1,2-a]pyrimidin-9-yl)ethyl)amino)benzoic acid;
(R)-2-((1-(3,7-dimethyl-4-oxo-2-(4-(5-(trifluoromethyl)benzo[d]thiazol-2-yl) piperazin-1-yl)-4H-pyrido[1,2-a]pyrimidin-9-yl)ethyl)amino)benzoic acid;
2-(((1R)-1-(3,7-dimethyl-4-oxo-2-(1,1,2,2-tetrafluoro-6-azaspiro[3.4]octan-6-yl)-4H-pyrido[1,2-a]pyrimidin-9-yl)ethyl)amino) benzoic acid;
(R)-2-((1-(2-(2,2-difluoro-6-azaspiro[3.4]octan-6-yl)-3,7-dimethyl-4-oxo-4H-pyrido[1,2-a]pyrimidin-9-yl)ethyl)amino)benzoic acid;
(R)-2-((1-(2-(4-(3-cyano-2-methylphenyl) piperazin-1-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(4-(3-(trifluoromethyl)phenyl) piperazin-1-yl)-4H-pyrido[1,2-a]pyrimidin-9-yl)ethyl)amino)benzoic acid;
(R)-2-((1-(2-(4-(2-fluorophenyl) piperazin-1-yl)-3,7-dimethyl-4-oxo-4H-pyrido[1,2-a]pyrimidin-9-yl)ethyl)amino)benzoic acid;
(R)-2-((1-(3,7-dimethyl-4-oxo-2-(4-(pyridin-4-yl) piperazin-1-yl)-4H-pyrido[1,2-a]pyrimidin-9-yl)ethyl)amino)benzoic acid;
(R)-2-((1-(3,7-dimethyl-4-oxo-2-(3-(5-(trifluoromethyl) isoxazol-3-yl)azetidin-1-yl)-4H-pyrido[1,2-a]pyrimidin-9-yl)ethyl)amino)benzoic acid;
2-(((R)-1-(2-((1R,4R)-5-(2,2-difluoroethyl)-2,5-diazabicyclo[2.2.1]heptan-2-yl)-3,7-dimethyl-4-oxo-4H-pyrido[1,2-a]pyrimidin-9-yl)ethyl)amino)benzoic acid;
2-(((1R)-1-(2-(5,5-difluorohexahydrocyclopenta[c]pyrrol-2(1H)-yl)-3,7-dimethyl-4-oxo-4H-pyrido[1,2-a]pyrimidin-9-yl)ethyl)amino)benzoic acid;
2-(((R)-1-(3,7-dimethyl-4-oxo-2-((1R,5S,6R)-6-(trifluoromethyl)-3-azabicyclo[3.1.0]hexan-3-yl)-4H-pyrido[1,2-a]pyrimidin-9-yl)ethyl)amino)benzoic acid;
(R)-2-((1-(3,7-dimethyl-2-(4-(4-methylthiazol-2-yl) piperazin-1-yl)-4-oxo-4H-pyrido[1,2-a]pyrimidin-9-yl)ethyl)amino)benzoic acid;
2-(((1R)-1-(2-(7,7-difluoro-2-azaspiro[4.4]nonan-2-yl)-3,7-dimethyl-4-oxo-4H-pyrido[1,2-a]pyrimidin-9-yl)ethyl)amino)benzoic acid; or
a pharmaceutically acceptable salt thereof.

21. A pharmaceutical composition comprising a compound or salt according to any of claims 1-20 together with a pharmaceutically acceptable carrier, excipient or diluent.

22. A method of treating a disease or disorder associated with modulation of phosphoinositide 3-kinase (PI3K), comprising administering to a patient in need thereof a therapeutically effective amount of a compound of any one of claims 1 to 20 or a pharmaceutical composition of claim 20.

23. The method of claim 22, wherein the PI3K is PI3Kα.

24. The method of claim 22 or claim 23, wherein the PI3K associated with the disease or disorder has a H1047R mutation.

25. The method of any one of claims 22 to 24, wherein the disease or disorder is a cancer.

26. The method of claim 25, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

27. The method of any one of claims 22 to 24, wherein the disease or disorder is CLOVES syndrome (congenital lipomatous overgrowth, vascular malformations, epidermal naevi, scoliosis/skeletal and spinal syndrome), or PIK3Cα-related overgrowth syndrome (PROS).

28. A method of inhibiting phosphoinositide 3-kinase (PI3K), comprising administering to a patient in need thereof a therapeutically effective amount of a compound of any one of claims 1 to 20 or a pharmaceutical composition of claim 21.

29. A method of treating cancer or a disorder, the method comprising administering to a patient in need thereof a therapeutically effective amount of a compound of any one of claims 1 to 20 or a pharmaceutical composition of claim 21.

30. The method of claim 29, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

31. The method of claim 27, wherein the disorder is CLOVES syndrome (congenital lipomatous overgrowth, vascular malformations, epidermal naevi, scoliosis/skeletal and spinal syndrome) or PIK3Cα-related overgrowth syndrome (PROS).

32. A method of treating cancer comprising administering a compound according to any of claims 1-20 or a pharmaceutical composition of claim 21 and a KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

33. A method treating cancer comprising administering a compound according to any of claims 1-20 or a pharmaceutical composition of claim 21 and a mutant selective KRAS inhibitor to a patient in need thereof, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.

34. A compound according to any of claims 1-20 or a pharmaceutical composition of claim 21 for use in the treatment of cancer in combination with a KRAS inhibitor, wherein the cancer is breast cancer, uterine carcinosarcoma, uterine endometrial carcinoma, colorectal adenocarcinoma, stomach adenocarcinoma, head and neck squamous cell carcinoma, cholangiocarcinoma, esophageal adenocarcinoma, bladder carcinoma, lung squamous cell carcinoma, brain glioma, adrenocortical carcinoma, liver hepatocellular carcinoma, sarcoma, prostate adenocarcinoma, kidney renal cell carcinoma, lung adenocarcinoma, ovarian cystadenocarcinoma, glioblastoma multiforme, melanoma.
